(19) United States
(12) Patent Application Publication
(10) Pub. No.: US 2025/0084074 A1
(43) Pub. Date: Mar. 13, 2025

(54) SUBSTITUTED HETEROCYCLES AS HSET INHIBITORS

(71) Applicants: MERCK PATENT GMBH, Darmstadt (DE); THE INSTITUTE OF CANCER RESEARCH, London (GB); CANCER RESEARCH HORIZONS, London (GB)

(72) Inventors: Hans-Peter BUCHSTALLER, Darmstadt (DE); Daniel KUHN, Darmstadt (DE); Ian COLLINS, London (GB); Thomas MATTHEWS, London (GB); Jan LANZ, London (GB); John CALDWELL, London (GB); Giampiero COLOMBANO, London (GB)

(73) Assignees: MERCK PATENT GMBH, Darmstadt (DE); THE INSTITUTE OF CANCER RESEARCH, London (GB); CANCER RESEARCH HORIZONS, London (GB)

(21) Appl. No.: 18/727,543

(22) PCT Filed: Jan. 9, 2023
(86) PCT No.: PCT/EP2023/050255

§ 371 Date: Jul. 9, 2024

(30) Foreign Application Priority Data
Jan. 10, 2022 (EP) 22150667.8

(51) Int. Cl. C07D 417/12; A61K 31/4245; A61K 31/426; A61K 31/427; A61K 31/454; A61K 31/4725; A61K 31/517; A61K 31/5377; C07D 277/56; C07D 413/12; C07D 413/14; C07D 417/14

(52) U.S. Cl. CPC C07D 417/12; A61K 31/4245; A61K 31/426; A61K 31/427; A61K 31/454; A61K 31/4725; A61K 31/517; A61K 31/5377; C07D 277/56; C07D 413/12; C07D 413/14; C07D 417/14

(57) ABSTRACT

The invention relates to substituted heterocycles of the general formula I,

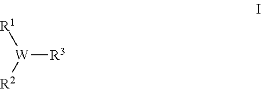

and the use of the compounds of the present invention for the treatment and/or prevention of hyperproliferative diseases and disorders in mammals, especially humans, and pharmaceutical compositions containing such compound.

The invention relates to substituted heterocycles of the general formula I,

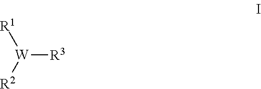

and the use of the compounds of the present invention for the treatment and/or prevention of hyperproliferative diseases and disorders such as cancer in mammals, especially humans, and pharmaceutical compositions containing such compounds.

### BACKGROUND OF THE INVENTION

DNA replication, followed by equal chromosome segregation, ensures the accurate transmission of the genetic information to daughter cells (Hall et al., 2003; Nigg, 2002; Zyss and Gergely, 2009). In most normal and malignant cells, centrosomes act as the dominant sites for spindle pole formation (Meunier and Vernos, 2012).

Centrosome duplication is also tightly controlled and occurs simultaneously with DNA replication, thereby ensuring the generation of two functional centrosomes that form the poles of the mitotic spindle (Sharp et al., 2000). In the assembly of a functional mitotic spindle, microtubule (MT) motor proteins play a central role (Cai et al., 2010; Ganem and Compton, 2004). One such protein, HSET (encoded by KIFC1 in humans and Kifc5a in mice), a minus-end MT motor, is of interest in cancer due to its impact on cell division (Cai et al., 2010; Goshima et al., 2005).

In recent years, the importance of centrosomes, and in particular HSET, for bipolar spindle formation has attracted much attention, although the precise role of HSET in this process remains a topic for debate (Mahoney et al., 2006; Tillement et al., 2009). Recent reports have linked centrosome amplification and high HSET expression to chromosome missegregation and aneuploidy, which are hallmarks of human cancer (Marx et al., 2009). Centrosome amplification disrupts asymmetric cell division in neuroblastoma cells and causes tumorigenesis in a fly model (Basto et al., 2008), and supernumerary centrosomes are also found in most solid tumor types, forming markers for aggressiveness in breast, brain, prostate, cervix, kidney, and bladder cancers (Chan, 2011). Hence, it is increasingly apparent that supernumerary centrosomes are not only indicative of malignancy but may also drive malignant transformation (Ogden et al., 2013). However, not all cells with centrosome amplification undergo multipolar mitosis, and a key mechanism by which cells with extra centrosomes achieve a pseudo-bipolar spindle is centrosome clustering (Basto et al., 2008; Ganem et al., 2009).

Although centrosome clustering prevents multipolar mitosis and cell death, it prolongs mitosis and increases the frequency of chromosome missegregation as a result of merotelic kinetochore attachments (Ganem et al., 2009; Kwon et al., 2008; Yang et al., 2008). Based on previous studies, centrosome clustering may prove to be the Achilles heel of cancer cells with supernumerary centrosomes (Basto et al., 2008), and a growing body of evidence suggests that inhibition of centrosome clustering could provide a new therapeutic strategy for tumors with a high incidence of centrosome amplification (Jordan and Wilson, 2004; Ogden et al., 2012).

A key protein that is known to be crucial for centrosome clustering is HSET (Ncd in flies). HSET is required by tumour cells to cluster supernumerary centrosomes (Basto et al., 2008; Kwon et al., 2008). HSET is a member of the Kinesin 14 family of MT motor proteins, which are force-generating enzymes that facilitate movement along MTs within the cell (Mountain et al., 1999) and which transport organelles, protein complexes and mRNAs along microtubules in an ATP-dependent fashion. HSET is a minus-end directed motor kinesin, that cross-links and slides microtubules exerting inward forces (Walczak et al., 1997; Cai et al., 2009; Rath et al., 2012). Although the precise role of HSET in cell division is not clear, previous evidence suggests that it is essential for the survival of cancer, but not normal, cells (Ganem et al., 2009; Kwon et al., 2008). High HSET expression levels are strongly correlated with metastasis of non-small cell lung cancer to the brain, pointing to an association between HSET, centrosome amplification, and tumorigenesis (Cai et al., 2010; Gordon et al., 2001; Grinberg-Rashi et al., 2009). Knockdown of HSET in normal retinal pigment epithelial 1 (RPE-1) cells or the breast cancer cell line MCF-7 (which does not have a high incidence of centrosome amplification) does not inhibit bipolar spindle formation, and cells undergo normal division (Kleylein-Sohn et al., 2012; Kwon et al., 2008). In contrast, knockdown of HSET in the supernumerary centrosome-containing breast cancer and neuroblastoma cell lines MDA-MB-231 and N1 E-115, respectively, prevents centrosome clustering and induces cell death by multipolar anaphases (Kwon et al., 2008). Hence, the above findings point to HSET as a target of interest in cancer treatment (Basto et al., 2008; Kraljevic Pavelic et al., 2011; Kramer et al., 2011; Kwon et al., 2008).

A number of studies have shown that HSET depletion increases cell death and the frequency of multipolarity in cells with supernumerary centrosomes, but not in cells with a normal number of centrosomes. For example, HSET depletion induces spindle multi-polarity and selectively sensitizes centrosome amplified ER-breast cancer cell lines, including triple negative breast cancer (TNBC), to cell death (Patel et al., 2018). Depletion of HSET was identified as inducing selective cytotoxicity in centrosome amplified cancer cells (Drosopoulos et al., 2014). In addition, HSET overexpression has been correlated with poor prognosis and resistance to docetaxel in breast cancer (De et al., 2009; Li et al., 2015), is observed in ovarian adenocarcinoma patients (Pawar et al., 2014) and in numerous other cancer types (Pannu et al., 2015). Furthermore, in non-small cell lung carcinoma (NSCLC) HSET expression was found to be highly predictive of the presence of brain metastasis in both early and advanced disease (Grinberg-Rashi et al., 2009).

A wide range of tumours, including centrosome amplified tumours, are treated by cytotoxic microtubule-targeted drugs (e.g. taxol, eribulin). Although inducing temporary remission, these drugs typically show severe side effects and the emergence of drug resistance leading to early relapse. More recently, agents targeting kinesin motor proteins, e.g. Eg5 inhibitors, have been explored to treat a variety of human tumours, which induce mono-polar spindles (the opposite phenotype to HSET inhibition), and target all rapidly dividing cells, including bone marrow cells. Consequently, they share dose-limiting toxicities with other antimitotic therapies. In contrast, an HSET inhibitor is anticipated to show reduced toxicity by selectively killing cells with centrosome amplification whereas cells with the normal number of centrosomes will remain unaffected (Ganem et al., 2009; Patel et al., 2015). These data together provide support for developing agents that selectively inhibit HSET to target centrosome-amplified tumours (Myers and Collins, 2016).

Examples of small molecule HSET inhibitors have been described in the literature. AZ82 is an ADP/ATP competitive inhibitor shown to be selective against a panel of nine other kinesins including Eg5 (Wu et al., 2013). AZ82 inhibited microtubule-stimulated HSET ATPase activity (IC₅₀=0.3 μM) in a biochemical assay and induced multipolar spindle formation and mitotic catastrophe in cells with amplified centrosomes. CW069 was an inhibitor of HSET (IC₅₀=75 μM) in biochemical assays (Watts et al., 2013). SR31527, also showed biochemical inhibition of HSET (IC₅₀=6.6 μM) (Zhang et al., 2016). More information can be found in WO09155025 and WO15085088.

Thus, there remains a need for therapies for the treatment and prevention of hyperproliferative diseases and disorders such as cancer. Therefore, the aim was to find HSET inhibitors that serve as potential therapeutics for the treatment of cancer diseases.

### SUMMARY OF THE INVENTION

Surprisingly, it has been found that the compounds according to the invention are highly selective and effective inhibitors of HSET and thus the compounds of the present invention can be used for the treatment of hyperproliferative diseases and disorders such as cancer.

The invention relates to the compounds of the general formula I,

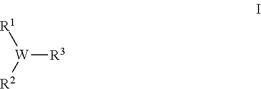

wherein
W denotes

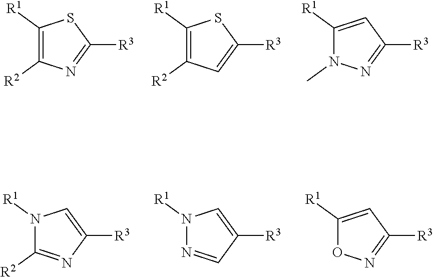

R¹ denotes Hal, CN, A, COOR⁷, CON(R⁷)₂, COA′ or Het¹,
R² denotes H, Hal or A′,
R³ denotes

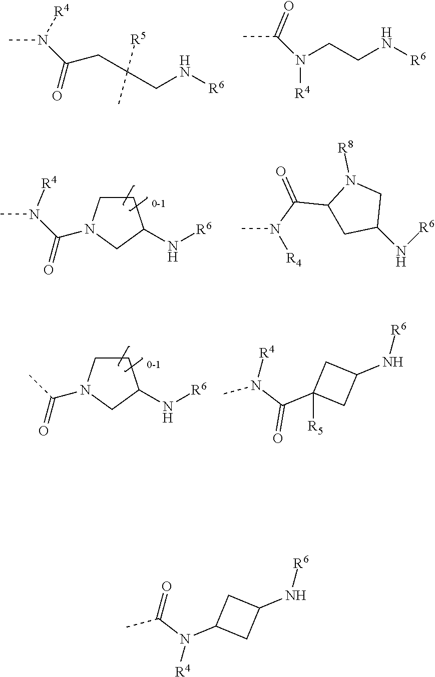

R⁴, R⁸ denote independently of one another H, unbranched or branched alkyl with 1-3° C.-atoms,
R⁵ denotes H or one or more substituents selected from the group of Hal, A, OH, OCH₃, NH₂, NHCH₃, N(CH₃)₂ and CH₂CN,
R⁶ denotes benzoyl, 2-isoquinolinyl or 4-quinazolinyl, which is unsubstituted or one-, two-, or threefold substituted with Hal, A, OH, N(R⁷)₂, COOR⁷, CN, NO₂ and/or or Het²,
A denotes unbranched or branched alkyl or cycloalkyl with 1-10 C-atoms, wherein two adjacent CH- and/or CH₂-groups may form a double bond and wherein one or two non-adjacent CH- and/or CH₂-groups may be replaced by N-, O- and/or S-atoms and wherein 1-7 H-atoms may be replaced by F or C,
A′ denotes unbranched or branched alkyl with 1-4 C-atoms, wherein 1-7 H-atoms may be replaced by F or Cl,
Het¹ denotes oxadiazolyl, which is unsubstituted or substituted with unbranched or branched alkyl with 1-4 C-atoms,
Het² denotes oxadiazolyl, tetrazolyl, pyrazolyl or oxazolyl, which unsubstituted or substituted with unbranched or branched alkyl with 1-4 C-atoms,
R⁷ denotes H or A,
Hal denotes F, Cl, Br or I and physiologically acceptable salts, derivatives, solvates, prodrugs and stereoisomers thereof, including mixtures thereof in all ratios.

The invention preferably relates to the compounds according to formula I, wherein W denotes

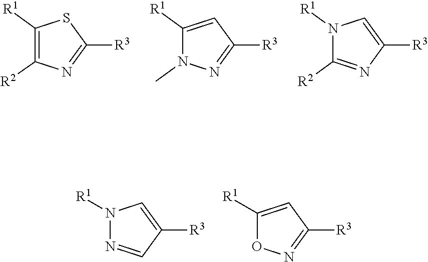

and R¹, R², R³, R⁴, R⁵, R⁶, R⁷, R⁸, A, A′, Het¹ and Het² have the meanings as in Claim 1, and physiologically acceptable salts, derivatives, solvates, prodrugs and stereoisomers thereof, including mixtures thereof in all ratios.

The invention more preferably relates to the compounds according to formula I, wherein W denotes

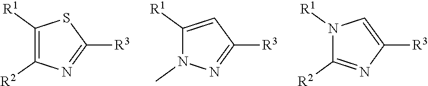

and R¹, R², R³, R⁴, R⁵, R⁶, R⁷, R⁸, A, A′, Het¹ and Het² have the meanings as in Claim 1, and physiologically acceptable salts, derivatives, solvates, prodrugs and stereoisomers thereof, including mixtures thereof in all ratios.

The invention particularly preferred relates to the compounds according to formula I, wherein W denotes

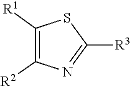

and R¹, R², R³, R⁴, R⁵, R⁶, R⁷, R⁸, A, A′, Het¹ and Het² have the meanings as in Claim 1, and physiologically acceptable salts, derivatives, solvates, prodrugs and stereoisomers thereof, including mixtures thereof in all ratios.

The invention preferably relates to the compounds according to formula I, wherein R² denotes H or A′ and W, R¹, R³, R⁴, R⁵, R⁶, R⁷, R⁸, A, A′, Het¹ and Het² have the meanings as in Claim 1, and physiologically acceptable salts, derivatives, solvates, prodrugs and stereoisomers thereof, including mixtures thereof in all ratios.

The invention more preferably relates to the compounds according to formula I, wherein R² denotes H or methyl and W, R¹, R³, R⁴, R⁵, R⁶, R⁷, R⁸, A, A′, Het¹ and Het² have the meanings as in Claim 1, and physiologically acceptable salts, derivatives, solvates, prodrugs and stereoisomers thereof, including mixtures thereof in all ratios.

The invention preferably relates to the compounds according to formula I, wherein R³ denotes

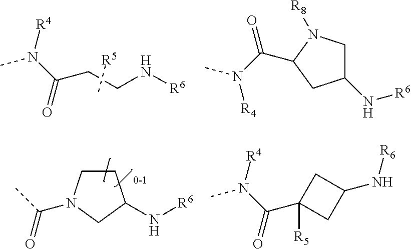

and W, R¹, R², R⁴, R⁵, R⁶, R⁷, R⁸, A, A′, Het¹ and Het² have the meanings as in Claim 1, and physiologically acceptable salts, derivatives, solvates, prodrugs and stereoisomers thereof, including mixtures thereof in all ratios.

The invention particularly preferred relates to the compounds according to formula I, wherein R³ denotes

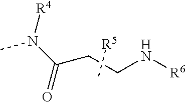

and W, R¹, R², R⁴, R⁵, R⁶, R⁷, R⁸, A, A′, Het¹ and Het² have the meanings as in Claim 1, and physiologically acceptable salts, derivatives, solvates, prodrugs and stereoisomers thereof, including mixtures thereof in all ratios.

The invention particularly preferred relates to the compounds according to formula I, wherein R⁶ denotes benzoyl or 2-isoquinolinyl and W, R¹, R², R³, R⁴, R⁵, R⁷, R⁸, A, A′, Het¹ and Het² have the meanings as in Claim 1, and physiologically acceptable salts, derivatives, solvates, prodrugs and stereoisomers thereof, including mixtures thereof in all ratios.

The invention preferably relates to the compounds according to formula I, wherein Het² denotes oxadiazolyl or tetrazolyl and W, R¹, R², R³, R⁴, R⁵, R⁶, R⁷, R⁸, A, A′, and Het¹ have the meanings as in Claim 1, and physiologically acceptable salts, derivatives, solvates, prodrugs and stereoisomers thereof, including mixtures thereof in all ratios.

The invention particularly preferred relates to the compounds according to formula I, wherein Het² denotes methyl-oxadiazolyl or methyl-tetrazolyl and W, R¹, R², R³, R⁴, R⁵, R⁶, R⁷, R⁸, A, A′, and Het¹ have the meanings as in Claim 1, and physiologically acceptable salts, derivatives, solvates, prodrugs and stereoisomers thereof, including mixtures thereof in all ratios.

The invention preferably relates to a compound selected from the group consisting of:

| | |
|---|---|
| 1 | Ethyl-4-methyl-2-(3-(3-methylbenzamido)propanamido)thiazole-5- |
| | carboxylate |
| 2 | Ethyl-4-methyl-2-(3-(3-(pyrrolidin-1- |
| | yl)benzamido)propanamido)thiazole-5-carboxylate |
| 3 | Ethyl-2-(3-(3-(1H-pyrazol-1-yl)benzamido)propanamido)-4- |
| | methylthiazole-5-carboxylate |
| 4 | Ethyl-4-methyl-2-(3-(3-(5-methyl-1,2,4-oxadiazol-3- |
| | yl)benzamido)propanamido)thiazole-5-carboxylate |
| 5 | Ethyl-4-methyl-2-(3-(5,6,7,8-tetrahydronaphthalene-2- |
| | carboxamido)propanamido)thiazole-5-carboxylate |
| 6 | Ethyl-4-methyl-2-(3-(3-(oxazol-5- |
| | yl)benzamido)propanamido)thiazole-5-carboxylate |
| 7 | Ethyl-4-methyl-2-(3-(3- |
| | morpholinobenzamido)propanamido)thiazole-5-carboxylate |
| 8 | Ethyl-(S)-2-(2-hydroxy-3-(3-methylbenzamido)propanamido)-4- |
| | methylthiazole-5-carboxylate |
| 9 | Ethyl-2-[3-[(3-methoxycarbonylbenzoyl)amino]propanoylamino]-4- |
| | methyl-thiazole-5-carboxylate |
| 10 | Ethyl-2-[3-[(3-fluoro-5-methoxycarbonyl-benzoyl)amino]- |
| | propanoylamino]-4-methyl-thiazole-5-carboxylate |
| 11 | Ethyl-2-[3-[(2-fluoro-3-methoxycarbonyl-benzoyl)amino]- |
| | propanoylamino]-4-methyl-thiazole-5-carboxylate |
| 12 | Ethyl-2-[3-[(2-chloro-5-methoxycarbonyl-benzoyl)amino]- |
| | propanoylamino]-4-methyl-thiazole-5-carboxylate |
| 13 | Ethyl-2-[3-[(3-chloro-5-methoxycarbonyl-benzoyl)amino]- |
| | propanoylamino]-4-methyl-thiazole-5-carboxylate |
| 14 | Ethyl-2-[3-[(3-methoxycarbonyl-4-methyl-benzoyl)amino]- |
| | propanoylamino]-4-methyl-thiazole-5-carboxylate |
| 15 | Ethyl-2-[3-[(3-methoxycarbonyl-5-methyl-benzoyl)amino]- |
| | propanoylamino]-4-methyl-thiazole-5-carboxylate |
| 16 | Ethyl-2-[3-[(3-methoxy-5-methoxycarbonyl-benzoyl)amino]- |
| | propanoylamino]-4-methyl-thiazole-5-carboxylate |
| 17 | Ethyl-2-[3-[(3-cyano-5-methoxycarbonyl-benzoyl)amino]- |
| | propanoylamino]-4-methyl-thiazole-5-carboxylate |
| 18 | Ethyl-2-[3-[(3-bromo-5-methoxycarbonyl-benzoyl)amino]- |
| | propanoylamino]-4-methyl-thiazole-5-carboxylate |
| 19 | Ethyl-2-[3-[(3-ethyl-5-methoxycarbonyl-benzoyl)amino]- |
| | propanoylamino]-4-methyl-thiazole-5-carboxylate |
| 20 | Ethyl-2-[3-[(3-hydroxy-5-methoxycarbonyl-benzoyl)amino]- |
| | propanoylamino]-4-methyl-thiazole-5-carboxylate |
| 21 | Ethyl-4-methyl-2-[3-[[3-(5-methyl-1,2,4-oxadiazol-3-yl)benzoyl]- |
| | amino]propylamino]thiazole-5-carboxylate |
| 22 | Ethyl-2-[3-[3-methoxycarbonyl-5-[(E)- |
| | styryl]benzoyl]amino]propanoyl-amino]-4-methyl-thiazole-5- |
| | carboxylate |
| 23 | Ethyl-2-(3-(3-(1,2,4-oxadiazol-3-yl)benzamido)propanamido)-4- |
| | methylthiazole-5-carboxylate |
| 24 | Ethyl-2-(3-(3-chloro-5-(5-methyl-1,2,4-oxadiazol-3-yl)benzamido)- |
| | propanamido)-4-methylthiazole-5-carboxylate |
| 25 | Propyl-2-(3-(3-chloro-5-(5-methyl-1,2,4-oxadiazol-3- |
| | yl)benzamido)-propanamido)-4-methylthiazole-5-carboxylate |
| 26 | Propyl-4-methyl-2-(3-(3-methyl-5-(5-methyl-1,2,4-oxadiazol-3- |
| | yl)benzamido)propanamido)thiazole-5-carboxylate |
| 27 | Propyl-1-methyl-3-(3-(3-methyl-5-(5-methyl-1,2,4-oxadiazol-3- |
| | yl)benzamido)propanamido)-1H-pyrazole-5-carboxylate |
| 28 | Propyl-2-(3-(3-cyano-5-(trifluoromethyl)benzamido)propanamido)- |
| | 4-methylthiazole-5-carboxylate |
| 29 | Propyl-2-(3-(3-(tert-butyl)-5-(methoxycarbonyl)benzamido)- |
| | propanamido)-4-methylthiazole-5-carboxylate |
| 30 | Propyl-2-(3-(3-cyano-5- |
| | (methoxycarbonyl)benzamido)propanamido)-4-methylthiazole-5- |
| | carboxylate |
| 31 | N-[3-[(5-tert-butyl-4-methyl-thiazol-2-yl)amino]-3-oxo-propyl]-3- |
| | chloro-5-(5-methyl-1,2,4-oxadiazol-3-yl)benzamide |
| 32 | Propyl-4-methyl-2-(3-(3-(5-methyl-1,2,4-oxadiazol-3-yl)-5- |
| | (trifluoromethyl)benzamido)propanamido)thiazole-5-carboxylate |
| 33 | Propyl-2-[3-[(3-methoxycarbonyl-5-nitro-benzoyl)amino]- |
| | propanoylamino]-4-methyl-thiazole-5-carboxylate |
| 34 | Ethyl-4-methyl-2-(3-(3-(3-methyl-1,2,4-oxadiazol-5- |
| | yl)benzamido)propanamido)thiazole-5-carboxylate |
| 35 | Cyclopropyl-4-methyl-2-(3-(3-(5-methyl-1,2,4-oxadiazol-3- |
| | yl)benzamido)propanamido)thiazole-5-carboxylate |
| 36 | Ethyl-2-(3-(3-(5-ethyl-1,2,4-oxadiazol-3- |
| | yl)benzamido)propanamido)-4-methylthiazole-5-carboxylate |
| 37 | Ethyl-4-methyl-2-(3-(3-(5-methyl-1,3,4-oxadiazol-2-yl)benzamido)- |
| | propanamido)thiazole-5-carboxylate |
| 38 | Ethyl-4-methyl-2-(3-(3-(2-methyl-2H-tetrazol-5-yl)benzamido)- |
| | propanamido)thiazole-5-carboxylate |
| 39 | Tert-butyl-4-methyl-2-[3-[[3-(2-methyltetrazol-5-yl)benzoyl]amino]- |
| | propanoylamino]thiazole-5-carboxylate |
| 40 | Isopropyl-4-methyl-2-[3-[3-(2-methyltetrazol-5-yl)benzoyl]amino]- |
| | propanoylamino]thiazole-5-carboxylate |
| 41 | N-[3-[(5-tert-butyl-4-methyl-thiazol-2-yl)amino]-3-oxo-propyl]-3-(2- |
| | methyltetrazol-5-yl)benzamide |
| 42 | Ethyl-2-(3-(3-(ethoxycarbonyl)benzamido)propanamido)-4- |
| | methylthiazole-5-carboxylate |
| 43 | Ethyl4-methyl-2-(3-(3-methyl-5-(5-methyl-1,2,4-oxadiazol-3- |
| | yl)benzamido)propanamido)thiazole-5-carboxylate |
| 44 | Propyl-2-(3-(3-(methoxycarbonyl)-5-(5-methyl-1,2,4-oxadiazol-3- |
| | yl)benzamido)propanamido)-4-methylthiazole-5-carboxylate |
| 45 | Ethyl-4-methyl-2-[3-[[7-(5-methyl-1,2,4-oxadiazol-3-yl)-1- |
| | isoquinolyl]amino]propanoylamino]thiazole-5-carboxylate |
| 46 | Methyl-4-methyl-2-[3-[7-(5-methyl-1,2,4-oxadiazol-3-yl)-1- |
| | isoquinolyl]amino]propanoylamino]thiazole-5-carboxylate |
| 47 | N-(5-tert-butyl-4-methyl-thiazol-2-yl)-3-[6-(5-methyl-1,2,4- |
| | oxadiazol-3-yl)quinazolin-4-yl]amino]propanamide |
| 48 | Tert-butyl-4-methyl-2-((1s,3s)-3-((6-(5-methyl-1,2,4-oxadiazol-3- |
| | yl)quinazolin-4-yl)amino)cyclobutane-1-carboxamido)thiazole-5- |
| | carboxylate |
| 49 | Tert-butyl-4-methyl-2-((1s,3s)-N-methyl-3-((6-(5-methyl-1,2,4- |
| | oxadiazol-3-yl)quinazolin-4-yl)amino)cyclobutane-1- |
| | carboxamido)thiazole-5-carboxylate |
| 50 | N-(5-tert-butyl-4-methyl-thiazol-2-yl)-3-[(7-cyano-1- |
| | isoquinolyl)amino]-propanamide |
| 51 | 3-[(7-cyano-1-isoquinolyl)amino]-N-[4-methyl-5-[1- |
| | (trifluoromethyl)-cyclopropyl]thiazol-2-yl]propanamide |
| 52 | 3-[[7-(5-methyl-1,2,4-oxadiazol-3-yl)-1-isoquinolyl]amino]-N-[4- |
| | methyl-5-[1-(trifluoromethyl)cyclopropyl]thiazol-2-yl]propanamide |
| 53 | N-(5-tert-butyl-4-methyl-thiazol-2-yl)-3-[(7-cyano-5-fluoro-1- |
| | isoquinolyl)amino]propanamide |
| 54 | Propyl-4-methyl-2-(3-((7-(5-methyl-1,2,4-oxadiazol-3- |
| | yl)isoquinolin-1-yl)amino)propanamido)thiazole-5-carboxylate |
| 55 | N-(5-cyanothiazol-2-yl)-3-((7-(5-methyl-1,2,4-oxadiazol-3- |
| | yl)isoquinolin-1-yl)amino)propanamide |
| 56 | 3-((7-(5-methyl-1,2,4-oxadiazol-3-yl)isoquinolin-1-yl)amino)-N-(1- |
| | methyl-5-(trifluoromethyl)-1H-pyrazol-3-yl)propanamide |
| 57 | 3-((7-(5-methyl-1,2,4-oxadiazol-3-yl)isoquinolin-1-yl)amino)-N-(1- |
| | methyl-5-pentyl-1H-pyrazol-3-yl)propanamide |
| 58 | N-(5-(tert-butyl)-4-methylthiazol-2-yl)-3-((7-(5-methyl-2H-tetrazol- |
| | 2-yl)isoquinolin-1-yl)amino)propanamide |
| 59 | N-(3-((5-(tert-butyl)-4-methylthiazol-2-yl)amino)-3-oxopropyl)-3-(5- |
| | methyl-2H-tetrazol-2-yl)benzamide |
| 60 | (S)-N-(1-((5-(tert-butyl)-4-methylthiazol-2-yl)amino)-6- |
| | (methylamino)-1-oxohexan-3-yl)-3-(5-methyl-2H-tetrazol-2- |
| | yl)benzamide |
| 61 | Ethyl-2-(3-(3-(5-methyl-1,2,4-oxadiazol-3-yl)benzamido)- |
| | propanamido)thiazole-5-carboxylate |
| 62 | Methyl-4-methyl-2-(3-(3-(5-methyl-1,2,4-oxadiazol-3- |
| | yl)benzamido)-propanamido)thiazole-5-carboxylate |
| 63 | Propyl-4-methyl-2-(3-(3-(5-methyl-1,2,4-oxadiazol-3- |
| | yl)benzamido)propanamido)thiazole-5-carboxylate |
| 64 | N-(3-((5-butyl-4-methylthiazol-2-yl)amino)-3-oxopropyl)-3-(5- |
| | methyl-1,2,4-oxadiazol-3-yl)benzamide |
| 65 | Ethyl-4-methyl-2-(3-methyl-3-(3-(5-methyl-1,2,4-oxadiazol-3- |
| | yl)benzamido)butanamido)thiazole-5-carboxylate |
| 66 | Isopropyl-4-methyl-2-(3-(3-(5-methyl-1,2,4-oxadiazol-3- |
| | yl)benzamido)-propanamido)thiazole-5-carboxylate |
| 67 | 2-methoxyethyl-4-methyl-2-(3-(3-(5-methyl-1,2,4-oxadiazol-3- |
| | yl)benzamido)propanamido)thiazole-5-carboxylate |
| 68 | Isobutyl-4-methyl-2-(3-(3-(5-methyl-1,2,4-oxadiazol-3- |
| | yl)benzamido)-propanamido)thiazole-5-carboxylate |
| 69 | 4-methoxybutyl-4-methyl-2-(3-(3-(5-methyl-1,2,4-oxadiazol-3- |
| | yl)benzamido)propanamido)thiazole-5-carboxylate |
| 70 | Propyl-4-methyl-2-((2-((7-(5-methyl-1,2,4-oxadiazol-3- |
| | yl)isoquinolin-1-yl)amino)ethyl)carbamoyl)thiazole-5-carboxylate |
| 71 | Ethyl-1-methyl-3-((2-((7-(5-methyl-1,2,4-oxadiazol-3-yl)isoquinolin- |
| | 1-yl)amino)ethyl)carbamoyl)-1H-pyrazole-5-carboxylate |
| 72 | Propyl-1-methyl-3-((2-((7-(5-methyl-1,2,4-oxadiazol-3- |
| | yl)isoquinolin-1-yl)amino)ethyl)carbamoyl)-1H-pyrazole-5- |
| | carboxylate |
| 73 | 1-methyl-3-(3-((7-(5-methyl-1,2,4-oxadiazol-3-yl)isoquinolin-1- |
| | yl)amino)propanamido)-N-propyl-1H-pyrazole-5-carboxamide |
| 74 | Propyl-4-methyl-2-(methyl(2-((7-(5-methyl-1,2,4-oxadiazol-3- |
| | yl)isoquinolin-1-yl)amino)ethyl)carbamoyl)thiazole-5-carboxylate |
| 75 | N-(5-(tert-butyl)-4-methylthiazol-2-yl)-3-((7-(5-methyl-1,2,4- |
| | oxadiazol-3-yl)isoquinolin-1-yl)amino)propanamide |
| 76 | N-(5-ethyl-1-methyl-1H-pyrazol-3-yl)-3-((7-(5-methyl-1,2,4- |
| | oxadiazol-3-yl)isoquinolin-1-yl)amino)propanamide |
| 77 | N-(5-(tert-butyl)isoxazol-3-yl)-3-((7-(5-methyl-1,2,4-oxadiazol-3- |
| | yl)isoquinolin-1-yl)amino)propanamide |
| 78 | N-(5-cyclopropylisoxazol-3-yl)-3-((7-(5-methyl-1,2,4-oxadiazol-3- |
| | yl)isoquinolin-1-yl)amino)propanamide |
| 79 | N-(5-ethylisoxazol-3-yl)-3-((7-(5-methyl-1,2,4-oxadiazol-3- |
| | yl)isoquinolin-1-yl)amino)propanamide |
| 80 | N-(5-(tert-butyl) thiazol-2-yl)-3-((7-(5-methyl-1,2,4-oxadiazol-3- |
| | yl)isoquinolin-1-yl)amino)propanamide |
| 81 | N-(5-(tert-butyl)-4-ethylthiazol-2-yl)-3-((7-(5-methyl-1,2,4- |
| | oxadiazol-3-yl)isoquinolin-1-yl)amino)propanamide |
| 82 | N-(5-(tert-butyl)-1-methyl-1H-pyrazol-3-yl)-3-((7-(5-methyl-1,2,4- |
| | oxadiazol-3-yl)isoquinolin-1-yl)amino)propanamide |
| 83 | N-[5-(3-methoxyoxetan-3-yl)-4-methyl-thiazol-2-yl]-3-[[7-(5-methyl- |
| | 1,2,4-oxadiazol-3-yl)-1-isoquinolyl]amino]propanamide |
| 84 | 3-[[7-(5-methyl-1,2,4-oxadiazol-3-yl)-1-isoquinolyl]amino]-N-[4- |
| | methyl-5-(3-propoxyoxetan-3-yl)thiazol-2-yl]propanamide |
| 85 | N,4-dimethyl-2-[3-[7-(5-methyl-1,2,4-oxadiazol-3-yl)-1- |
| | isoquinolyl]amino]propanoylamino]-N-propyl-thiazole-5- |
| | carboxamide |
| 86 | N-[5-(cyclohexen-1-yl) thiazol-2-yl]-3-[7-(5-methyl-1,2,4-oxadiazol- |
| | 3-yl)-1-isoquinolyl]amino]propanamide |
| 87 | N-[5-(1-methoxycyclohexyl)thiazol-2-yl]-3-[7-(5-methyl-1,2,4- |
| | oxadiazol-3-yl)-1-isoquinolyl]amino]propanamide |
| 88 | Ethyl-4-isopropyl-2-[3-[3-(5-methyl-1,2,4-oxadiazol-3- |
| | yl)benzoyl]amino]propanoylamino]thiazole-5-carboxylate |
| 89 | Tertbutyl-4-methyl-2-(methyl(2-((7-(5-methyl-1,2,4-oxadiazol-3- |
| | yl)isoquinolin-1-yl)amino)ethyl)carbamoyl)thiazole-5-carboxylate |
| 90 | Methyl-4-ethyl-2-[3-[3-(5-methyl-1,2,4-oxadiazol-3- |
| | yl)benzoyl]amino]propanoylamino]thiazole-5-carboxylate |
| 91 | Ethyl-2-[3-[3-(5-methyl-1,2,4-oxadiazol-3-yl)benzoyl]amino]- |
| | propanoylamino]-4-(trifluoromethyl)thiazole-5-carboxylate |
| 92 | Ethyl-4-ethyl-2-[3-[3-(5-methyl-1,2,4-oxadiazol-3- |
| | yl)benzoyl]amino]propanoylamino]thiazole-5-carboxylate |
| 93 | N-[3-[5-[(E)-2-cyclopentylvinyl]-4-methyl-thiazol-2-yl]amino]-3- |
| | oxo-propyl]-3-(5-methyl-1,2,4-oxadiazol-3-yl)benzamide |
| 94 | N-[3-[[5-[(E)-3-methoxyprop-1-enyl]-4-methyl-thiazol-2-yl]amino]- |
| | 3-oxo-propyl]-3-(5-methyl-1,2,4-oxadiazol-3-yl)benzamide |
| 95 | 3-(5-methyl-1,2,4-oxadiazol-3-yl)-N-[3-[4-methyl-5-(1- |
| | phenylvinyl)thiazol-2-yl]amino]-3-oxo-propyl]benzamide |
| 96 | 3-[7-(5-methyl-1,2,4-oxadiazol-3-yl)-1-isoquinolyl]amino]-N-[5- |
| | (1,1,2,2,2-pentafluoroethyl)thiazol-2-yl]cyclobutanecarboxamide |
| 97 | N-(5-bromo-4-methyl-thiazol-2-yl)-3-[[7-(5-methyl-1,2,4-oxadiazol- |
| | 3-yl)-1-isoquinolyl]amino]cyclobutanecarboxamide |
| 98 | (1s,3s)-N-(5-(tert-butyl)-4-methylthiazol-2-yl)-3-((7-(5-methyl-1,2,4- |
| | oxadiazol-3-yl)isoquinolin-1-yl)amino)cyclobutane-1-carboxamide |
| 99 | Propyl-4-methyl-2-((1s,3s)-3-((7-(5-methyl-1,2,4-oxadiazol-3- |
| | yl)isoquinolin-1-yl)amino)cyclobutane-1-carboxamido)thiazole-5- |
| | carboxylate |
| 100 | (1s,3s)-N-(5-(tert-butyl)isoxazol-3-yl)-3-((7-(5-methyl-1,2,4- |
| | oxadiazol-3-yl)isoquinolin-1-yl)amino)cyclobutane-1-carboxamide |
| 101 | (1s,3s)-3-((7-(5-methyl-1,2,4-oxadiazol-3-yl)isoquinolin-1- |
| | yl)amino)-N-(1-methyl-5-pentyl-1H-pyrazol-3-yl)cyclobutane-1- |
| | carboxamide |
| 102 | (1s,3s)-N-(5-isobutyl-4-methylthiophen-2-yl)-3-((7-(5-methyl-1,2,4- |
| | oxadiazol-3-yl)isoquinolin-1-yl)amino)cyclobutane-1-carboxamide |
| 103 | (1s,3s)-3-((7-(5-methyl-1,2,4-oxadiazol-3-yl)isoquinolin-1- |
| | yl)amino)-N-(4-methyl-5-(trifluoromethyl)thiazol-2-yl)cyclobutane- |
| | 1-carboxamide |
| 104 | Tert-butyl-4-methyl-2-((1s,3s)-3-(3-(5-methyl-1,2,4-oxadiazol-3- |
| | yl)benzamido)cyclobutane-1-carboxamido)thiazole-5-carboxylate |
| 105 | Tert-butyl-2-((1s,3s)-3-(3-(1,2,4-oxadiazol-3-yl)benzamido)- |
| | cyclobutane-1-carboxamido)-4-methylthiazole-5-carboxylate |
| 106 | Tert-butyl-4-methyl-2-((1s,3s)-3-(3-(5-methyl-1,3,4-oxadiazol-2- |
| | yl)benzamido)cyclobutane-1-carboxamido) thiazole-5-carboxylate |
| 107 | Tert-butyl-4-methyl-2-((1s,3s)-3-(3-(2-methyl-2H-tetrazol-5- |
| | yl)benzamido)cyclobutane-1-carboxamido) thiazole-5-carboxylate |
| 108 | Tert-butyl-2-((1s,3s)-3-((7-chloroisoquinolin-1- |
| | yl)amino)cyclobutane-1-carboxamido)-4-methylthiazole-5- |
| | carboxylate |
| 109 | Tert-butyl-4-methyl-2-((1s,3s)-3-((7-(trifluoromethyl)isoquinolin-1- |
| | yl)amino)cyclobutane-1-carboxamido)thiazole-5-carboxylate |
| 110 | Tert-butyl-4-methyl-2-[3-[7-(5-methyl-1,2,4-oxadiazol-3-yl)-1- |
| | isoquinolyl]amino]cyclobutanecarbonyl]amino]thiazole-5- |
| | carboxylate |
| 111 | Tert-butyl-4-methyl-2-[methyl-[3-[7-(5-methyl-1,2,4-oxadiazol-3- |
| | yl)-1-isoquinolyl]amino]cyclobutanecarbonyl]amino]thiazole-5- |
| | carboxylate |
| 112 | Tert-butyl-4-chloro-2-[3-[7-(5-methyl-1,2,4-oxadiazol-3-yl)-1- |
| | isoquinolyl]amino]cyclobutanecarbonyl]amino]thiazole-5- |
| | carboxylate |
| 113 | N-tert-butyl-4-chloro-2-[[3-[[7-(5-methyl-1,2,4-oxadiazol-3-yl)-1- |
| | isoquinolyl]amino]cyclobutanecarbonyl]amino]thiazole-5- |
| | carboxamide |
| 114 | [(1R)-2,2,2-trifluoro-1-methyl-ethyl]-4-methyl-2-[[3-[[7-(5-methyl- |
| | 1,2,4-oxadiazol-3-yl)-1-isoquinolyl]amino]cyclobutanecarbonyl] |
| | amino]-thiazole-5-carboxylate |
| 115 | [(1S)-2,2,2-trifluoro-1-methyl-ethyl]-4-methyl-2-[[3-[7-(5-methyl- |
| | 1,2,4-oxadiazol-3-yl)-1-isoquinolyl]amino]cyclobutanecarbonyl] |
| | amino]-thiazole-5-carboxylate |
| 116 | Ethyl-4-(difluoromethyl)-2-[[3-[[7-(5-methyl-1,2,4-oxadiazol-3-yl)-1- |
| | isoquinolyl]amino]cyclobutanecarbonyl]amino]thiazole-5- |
| | carboxylate |
| 117 | N-(5-tert-butyl-4-methyl-thiazol-2-yl)-3-[(7-methyl-1- |
| | isoquinolyl)amino]-cyclobutanecarboxamide |
| 118 | 3-(5-methyl-1,2,4-oxadiazol-3-yl)-N-(3-((4-methyl-5-(3-methyl- |
| | 1,2,4-oxadiazol-5-yl)thiazol-2-yl)amino)-3-oxopropyl)benzamide |
| 119 | 3-((7-(5-methyl-1,2,4-oxadiazol-3-yl)isoquinolin-1-yl)amino)-N-(5- |
| | (5-propyl-1,2,4-oxadiazol-3-yl)thiazol-2-yl)propanamide |
| 120 | Proyl-1-methyl-3-(3-(3-(5-methyl-1,2,4-oxadiazol-3- |
| | yl)benzamido)propanamido)-1H-pyrazole-5-carboxylate |
| 121 | Propyl-4-methyl-2-(N-methyl-3-(3-(5-methyl-1,2,4-oxadiazol-3- |
| | yl)benzamido)propanamido)thiazole-5-carboxylate |
| 122 | Cyclopentyl-4-methyl-2-(3-(3-(5-methyl-1,2,4-oxadiazol-3- |
| | yl)benzamido)propanamido)thiazole-5-carboxylate |
| 123 | Hexyl-4-methyl-2-(3-(3-(5-methyl-1,2,4-oxadiazol-3- |
| | yl)benzamido)propanamido)thiazole-5-carboxylate |
| 124 | Cyclohexyl-4-methyl-2-(3-(3-(5-methyl-1,2,4-oxadiazol-3- |
| | yl)benzamido)propanamido)thiazole-5-carboxylate |
| 125 | 4-aminobutyl-4-methyl-2-(3-(3-(5-methyl-1,2,4-oxadiazol-3- |
| | yl)benzamido)propanamido)thiazole-5-carboxylate |
| 126 | 3-(4-methoxyphenyl)propyl-4-methyl-2-(3-(3-(5-methyl-1,2,4- |
| | oxadiazol-3-yl)benzamido)propanamido)thiazole-5-carboxylate |
| 127 | 4-(2-methoxyethoxy)butyl-4-methyl-2-(3-(3-(5-methyl-1,2,4- |
| | oxadiazol-3-yl)benzamido)propanamido)thiazole-5-carboxylate |
| 128 | Methyl-2-methyl-5-[3-[3-(5-methyl-1,2,4-oxadiazol-3- |
| | yl)benzoyl]amino]propanoylamino]pyrazole-3-carboxylate |
| 129 | 3-(5-methyl-1,2,4-oxadiazol-3-yl)-N-(3-((4-methyl-5-(pentan-2- |
| | yl)thiazol-2-yl)amino)-3-oxopropyl)benzamide |
| 130 | 4-acetamidobutyl-4-methyl-2-(3-(3-(5-methyl-1,2,4-oxadiazol-3- |
| | yl)benzamido)propanamido)thiazole-5-carboxylate |
| 131 | Propyl-1-methyl-3-(3-((7-(5-methyl-1,2,4-oxadiazol-3- |
| | yl)isoquinolin-1-yl)amino)propanamido)-1H-pyrazole-5-carboxylate |
| 132 | Tert-butyl-4-methyl-2-(3-(3-(5-methyl-1,2,4-oxadiazol-3- |
| | yl)benzamido)propanamido)thiazole-5-carboxylate |
| 133 | Ethyl-3-methyl-5-[3-[3-(5-methyl-1,2,4-oxadiazol-3- |
| | yl)benzoyl]amino]propanoylamino]thiophene-2-carboxylate |
| 134 | Ethyl-4-methyl-2-(3-(3-(5-methyl-1,2,4-oxadiazol-3-yl)benzamido)- |
| | N-propylpropanamido)thiazole-5-carboxylate |
| 135 | Ethyl-3-(3-((7-(5-methyl-1,2,4-oxadiazol-3-yl)isoquinolin-1- |
| | yl)amino)propanamido)-1H-pyrazole-5-carboxylate. |
| 136 | 6-hydroxyhexyl-4-methyl-2-(3-(3-(5-methyl-1,2,4-oxadiazol-3-yl)-5- |
| | (trifluoromethyl)benzamido)propanamido)thiazole-5-carboxylate |
| 137 | 5-butyl-4-methyl-N-(2-((7-(5-methyl-1,2,4-oxadiazol-3- |
| | yl)isoquinolin-1-yl)amino)ethyl)thiazole-2-carboxamide |
| 138 | Cyclopentyl-1-methyl-3-(3-((7-(5-methyl-1,2,4-oxadiazol-3- |
| | yl) isoquinolin-1-yl)amino)propanamido)-1H-pyrazole-5-carboxylate |
| 139 | 3-{[3-(5-methyl-1,2,4-oxadiazol-3-yl)phenyl]formamido}-N-[4- |
| | methyl-5-(trifluoromethyl)-1,3-thiazol-2-yl]propenamide |
| 140 | Ethyl-2-((2-(3-(5-ethyl-1,2,4-oxadiazol-3-yl)benzamido)ethyl)- |
| | carbamoyl)-4-methylthiazole-5-carboxylate |
| 141 | Ethyl-4-methyl-2-((2-(3-(5-methyl-1,2,4-oxadiazol-3- |
| | yl)benzamido)ethyl)carbamoyl)thiazole-5-carboxylate |
| 142 | Ethyl-(R)-4-methyl-2-(3-(3-(5-methyl-1,2,4-oxadiazol-3- |
| | yl)benzamido)butanamido)thiazole-5-carboxylate |
| 143 | Ethyl-(S)-4-methyl-2-(3-(3-(5-methyl-1,2,4-oxadiazol-3- |
| | yl)benzamido)butanamido)thiazole-5-carboxylate |
| 144 | Propyl-2-(2-(4-(3-chloro-5-(5-methyl-1,2,4-oxadiazol-3- |
| | yl)benzamido)-1-methylpiperidin-4-yl)acetamido)-4-methylthiazole- |
| | 5-carboxylate |
| 145 | Ethyl-4-methyl-2-(2-methyl-3-(3-(5-methyl-1,2,4-oxadiazol-3- |
| | yl)benzamido)propanamido)thiazole-5-carboxylate |
| 146 | 4-(3-((5-(ethoxycarbonyl)-4-methylthiazol-2-yl)amino)-1-(3-(5- |
| | methyl-1,2,4-oxadiazol-3-yl)benzamido)-3-oxopropyl)piperidin-1- |
| | ium |
| 147 | 4-(3-((5-(ethoxycarbonyl)-4-methylthiazol-2-yl)amino)-1-(3-(5- |
| | methyl-1,2,4-oxadiazol-3-yl)benzamido)-3-oxopropyl)-1- |
| | methylpiperidin-1-ium |
| 148 | Ethyl-2-[[(3R)-4-amino-3-[3-(5-methyl-1,2,4-oxadiazol-3- |
| | yl)benzoyl]amino]butanoyl]amino]-4-methyl-thiazole-5-carboxylate |
| 149 | Ethyl-4-methyl-2-[(3S,4R)-4-[3-(5-methyl-1,2,4-oxadiazol-3- |
| | yl)benzoyl]amino]pyrrolidine-3-carbonyl]amino]thiazole-5- |
| | carboxylate; |
| 150 | Ethyl-(S)-2-(6-amino-3-(3-(5-methyl-1,2,4-oxadiazol-3- |
| | yl)benzamido)hexanamido)-4-methylthiazole-5-carboxylate |
| 151 | Propyl-(S)-2-(6-amino-3-(3-(5-methyl-1,2,4-oxadiazol-3- |
| | yl)benzamido)hexanamido)-4-methylthiazole-5-carboxylate |
| 152 | Propyl-4-methyl-2-[[(3S)-6-(methylamino)-3-[3-(5-methyl-1,2,4- |
| | oxadiazol-3-yl)benzoyl]amino]hexanoyl]amino]thiazole-5- |
| | carboxylate |
| 153 | Propyl-(R)-4-methyl-2-(3-(3-(5-methyl-1,2,4-oxadiazol-3- |
| | yl)benzamido)-6-(methylamino)hexanamido)thiazole-5- |
| | carboxylate |
| 154 | Tertbutyl-4-methyl-2-[[(3S)-6-(methylamino)-3-[[3-(5-methyl-1,2,4- |
| | oxadiazol-3-yl)benzoyl]amino]hexanoyl]amino]thiazole-5- |
| | carboxylate |
| 155 | Cyclopentyl-S)-4-methyl-2-(3-(3-(5-methyl-1,2,4-oxadiazol-3- |
| | yl)benzamido)-6-(methylamino)hexanamido)thiazole-5- |
| | carboxylate |
| 156 | Isopropyl-(S)-4-methyl-2-(3-(3-(5-methyl-1,2,4-oxadiazol-3- |
| | yl)benzamido)-6-(methylamino)hexanamido)thiazole-5- |
| | carboxylate |
| 157 | (S)-N-(1-((5-(tert-butyl)-4-methylthiazol-2-yl)amino)-6- |
| | (methylamino)-1-oxohexan-3-yl)-3-chloro-5-(5-methyl-1,2,4- |
| | oxadiazol-3-yl)benzamide |
| 158 | Propyl-(S)-2-(6-(dimethylamino)-3-(3-(5-methyl-1,2,4-oxadiazol-3- |
| | yl)benzamido)hexanamido)-4-methylthiazole-5-carboxylate |
| 159 | Propyl-(S)-4-methyl-2-(3-(3-(5-methyl-1,2,4-oxadiazol-3- |
| | yl)benzamido)-6-morpholinohexanamido)thiazole-5-carboxylate |
| 160 | Propyl-(S)-2-(2-amino-3-(3-(5-methyl-1,2,4-oxadiazol-3- |
| | yl)benzamido)propanamido)-4-methylthiazole-5-carboxylate |
| 161 | (S)-N-(2-amino-3-((5-(tert-butyl)-4-ethylthiazol-2-yl)amino)-3- |
| | oxopropyl)-3-(5-methyl-1,2,4-oxadiazol-3-yl)benzamide |
| 162 | Propyl-(S)-2-(5-(dimethylamino)-3-(3-(5-methyl-1,2,4-oxadiazol-3- |
| | yl)benzamido)pentanamido)-4-methylthiazole-5-carboxylate |
| 163 | Propyl-(S)-2-(6-amino-3-((7-(5-methyl-1,2,4-oxadiazol-3- |
| | yl) isoquinolin-1-yl)amino)hexanamido)-4-methylthiazole-5- |
| | carboxylate |
| 164 | (S)-6-amino-N-(5-(tert-butyl)-4-methylthiazol-2-yl)-3-((7-(5-methyl- |
| | 1,2,4-oxadiazol-3-yl)isoquinolin-1-yl)amino)hexanamide |
| 165 | Cyclopentyl-(S)-4-methyl-2-(3-((7-(5-methyl-1,2,4-oxadiazol-3- |
| | yl)isoquinolin-1-yl)amino)-6-(methylamino)hexanamido)thiazole-5- |
| | carboxylate |
| 166 | 1,3-difluoropropan-2-yl (S)-4-methyl-2-(3-((7-(5-methyl-1,2,4- |
| | oxadiazol-3-yl)isoquinolin-1-yl)amino)-6-(methylamino)- |
| | hexanamido)thiazole-5-carboxylate |
| 167 | Isopropyl-(S)-4-methyl-2-(3-((7-(5-methyl-1,2,4-oxadiazol-3- |
| | yl)isoquinolin-1-yl)amino)-6-(methylamino)hexanamido)thiazole-5- |
| | carboxylate |
| 168 | Tertbutyl-(S)-4-methyl-2-(3-((7-(5-methyl-1,2,4-oxadiazol-3- |
| | yl)isoquinolin-1-yl)amino)-6-(methylamino)hexanamido)thiazole-5- |
| | carboxylate |
| 169 | (S)-4-methyl-2-(3-((7-(5-methyl-1,2,4-oxadiazol-3-yl)isoquinolin-1- |
| | yl)amino)-6-(methylamino)hexanamido)thiazole-5-carboxylic |
| 170 | 3,3-difluorocyclopentyl-4-methyl-2-((S)-3-((7-(5-methyl-1,2,4- |
| | oxadiazol-3-yl)isoquinolin-1-yl)amino)-6-(methylamino) |
| | hexanamido)-thiazole-5-carboxylate |
| 171 | (S)-N-(5-(tert-butyl)-4-methylthiazol-2-yl)-3-((7-(5-methyl-1,2,4- |
| | oxadiazol-3-yl)isoquinolin-1-yl)amino)-6-(methylamino)hexanamide |
| 172 | Propyl-(S)-4-methyl-2-(3-((7-(5-methyl-1,2,4-oxadiazol-3- |
| | yl)isoquinolin-1-yl)amino)-6-(methylamino)hexanamido)thiazole-5- |
| | carboxylate |
| 173 | Propyl-(R)-2-(2-amino-3-(3-(5-methyl-1,2,4-oxadiazol-3- |
| | yl)benzamido)propanamido)-4-methylthiazole-5-carboxylate |
| 174 | Propyl-(S)-2-(2-amino-3-((7-(5-methyl-1,2,4-oxadiazol-3- |
| | yl)isoquinolin-1-yl)amino)propanamido)-4-methylthiazole-5- |
| | carboxylate |
| 175 | Propyl-(S)-2-(2-(dimethylamino)-3-(3-(5-methyl-1,2,4-oxadiazol-3- |
| | yl)benzamido)propanamido)-4-methylthiazole-5-carboxylate |
| 176 | Propyl-(R)-2-(2-hydroxy-3-((7-(5-methyl-1,2,4-oxadiazol-3- |
| | yl)isoquinolin-1-yl)amino)propanamido)-4-methylthiazole-5- |
| | carboxylate |
| 177 | Tertbutyl-(R)-2-(2-hydroxy-3-((7-(5-methyl-1,2,4-oxadiazol-3- |
| | yl)isoquinolin-1-yl)amino)propanamido)-4-methylthiazole-5- |
| | carboxylate |
| 178 | Propyl-(S)-2-(2-hydroxy-3-((7-(5-methyl-1,2,4-oxadiazol-3- |
| | yl)isoquinolin-1-yl)amino)propanamido)-4-methylthiazole-5- |
| | carboxylate |
| 179 | (S)-2-hydroxy-N-(5-isobutyl-4-methylthiazol-2-yl)-3-((7-(5-methyl- |
| | 1,2,4-oxadiazol-3-yl)isoquinolin-1-yl)amino)propanamide |
| 180 | Tertbutyl-(S)-2-(2-hydroxy-3-((7-(5-methyl-1,2,4-oxadiazol-3- |
| | yl)isoquinolin-1-yl)amino)propanamido)-4-methylthiazole-5- |
| | carboxylate |
| 181 | Tertbutyl-(S)-2-(2-hydroxy-3-((7-(1-methyl-1H-pyrazol-4- |
| | yl)isoquinolin-1-yl)amino)propanamido)-4-methylthiazole-5- |
| | carboxylate |
| 182 | (S)-2-hydroxy-3-((7-(5-methyl-1,2,4-oxadiazol-3-yl)isoquinolin-1- |
| | yl)amino)-N-(1-methyl-5-(5-propyl-1,2,4-oxadiazol-3-yl)-1H- |
| | pyrazol-3-yl)propanamide |
| 183 | (S)-2-hydroxy-N-(1-methyl-5-(5-propyl-1,2,4-oxadiazol-3-yl)-1H- |
| | pyrazol-3-yl)-3-((7-methylisoquinolin-1-yl)amino)propanamide |
| 184 | Tertbutyl-(R)-2-(2-hydroxy-3-((7-methylisoquinolin-1- |
| | yl)amino)propanamido)-4-methylthiazole-5-carboxylate |
| 185 | Tertbutyl-(S)-2-(2-hydroxy-3-((7-methylisoquinolin-1- |
| | yl)amino)propanamido)-4-methylthiazole-5-carboxylate |
| 186 | Propyl(S)-2-(2-methoxy-3-((7-(5-methyl-1,2,4-oxadiazol-3- |
| | yl)isoquinolin-1-yl)amino)propanamido)-4-methylthiazole-5- |
| | carboxylate |
| 187 | (S)-N-(5-(tert-butyl)-4-methylthiazol-2-yl)-2-hydroxy-3-((7-(5- |
| | methyl-1,2,4-oxadiazol-3-yl)isoquinolin-1-yl)amino)propanamide |
| 188 | (S)-2-hydroxy-3-((7-(5-methyl-1,2,4-oxadiazol-3-yl)isoquinolin-1- |
| | yl)amino)-N-(1-methyl-5-pentyl-1H-pyrazol-3-yl)propanamide |
| 189 | (R)-2-hydroxy-3-((7-(5-methyl-1,2,4-oxadiazol-3-yl)isoquinolin-1- |
| | yl)amino)-N-(1-methyl-5-pentyl-1H-pyrazol-3-yl)propanamide |
| 190 | (R)-N-(5-(tert-butyl)-4-methylthiazol-2-yl)-2-hydroxy-3-((7-(5- |
| | methyl-1,2,4-oxadiazol-3-yl)isoquinolin-1-yl)amino)propanamide |
| 191 | (R)-2-hydroxy-N-(5-isobutyl-4-methylthiazol-2-yl)-3-((7-(5-methyl- |
| | 1,2,4-oxadiazol-3-yl)isoquinolin-1-yl)amino)propanamide |
| 192 | (R)-2-hydroxy-3-((7-(5-methyl-1,2,4-oxadiazol-3-yl)isoquinolin-1- |
| | yl)amino)-N-(4-methyl-5-(1-(trifluoromethyl)cyclopropyl)thiazol-2- |
| | yl)propanamide |
| 193 | Tertbutyl-2-[(3S)-4-cyano-3-{[3-(5-methyl-1,2,4-oxadiazol-3- |
| | yl)phenyl]formamido}butanamido]-4-methyl-1,3-thiazole-5- |
| | carboxylate |
| 194 | Tertbutyl-2-[(3R)-4-cyano-3-{[3-(5-methyl-1,2,4-oxadiazol-3- |
| | yl)phenyl]formamido}butanamido]-4-methyl-1,3-thiazole-5- |
| | carboxylate |
| 195 | Propan-2-yl-2-[(3S)-4-cyano-3-{[3-(5-methyl-1,2,4-oxadiazol-3- |
| | yl)phenyl]formamido}butanamido]-4-methyl-1,3-thiazole-5- |
| | carboxylate |
| 196 | Propan-2-yl-2-[(3R)-4-cyano-3-{[3-(5-methyl-1,2,4-oxadiazol-3- |
| | yl)phenyl]formamido}butanamido]-4-methyl-1,3-thiazole-5- |
| | carboxylate |
| 197 | Methyl-2-[(3S)-4-cyano-3-{[3-(5-methyl-1,2,4-oxadiazol-3- |
| | yl)phenyl]formamido}butanamido]-4-methyl-1,3-thiazole-5- |
| | carboxylate |
| 198 | Methyl-2-[(3R)-4-cyano-3-{[3-(5-methyl-1,2,4-oxadiazol-3- |
| | yl)phenyl]formamido}butanamido]-4-methyl-1,3-thiazole-5- |
| | carboxylate |
| 199 | Propan-2-yl-2-[(3S)-4-cyano-N-methyl-3-{[3-(5-methyl-1,2,4- |
| | oxadiazol-3-yl)phenyl]formamido}butanamido]-4-methyl-1,3- |
| | thiazole-5-carboxylate |
| 200 | Propan-2-yl-2-[(3R)-4-cyano-N-methyl-3-{[3-(5-methyl-1,2,4- |
| | oxadiazol-3-yl)phenyl]formamido}butanamido]-4-methyl-1,3- |
| | thiazole-5-carboxylate |
| 201 | Tertbutyl-2-[(3S)-4-cyano-3-{[3-(1-methyl-1H-pyrazol-4-yl)phenyl]- |
| | formamido}butanamido]-4-methyl-1,3-thiazole-5-carboxylate |
| 202 | Tertbutyl-2-[(3R)-4-cyano-3-{[3-(1-methyl-1H-pyrazol-4-yl)phenyl]- |
| | formamido}butanamido]-4-methyl-1,3-thiazole-5-carboxylate |
| 203 | Propan-2-yl-2-[(3S)-4-cyano-3-{[3-(1-methyl-1H-pyrazol-4- |
| | yl)phenyl]formamido}butanamido]-4-methyl-1,3-thiazole-5- |
| | carboxylate |
| 204 | Propan-2-yl-2-[(3R)-4-cyano-3-{[3-(1-methyl-1H-pyrazol-4- |
| | yl)phenyl]formamido}butanamido]-4-methyl-1,3-thiazole-5- |
| | carboxylate |
| 205 | Propan-2-yl-2-[(3S)-4-cyano-N-methyl-3-{[3-(1-methyl-1H-pyrazol- |
| | 4-yl)phenyl]formamido}butanamido]-4-methyl-1,3-thiazole-5- |
| | carboxylate |
| 206 | Propan-2-yl-2-[(3R)-4-cyano-N-methyl-3-{[3-(1-methyl-1H-pyrazol- |
| | 4-yl)phenyl]formamido}butanamido]-4-methyl-1,3-thiazole-5- |
| | carboxylate |
| 207 | Propan-2-yl-2-[(3S)-4-cyano-3-[(3-cyanophenyl)formamido]- |
| | butanamido]-4-methyl-1,3-thiazole-5-carboxylate |
| 208 | Propan-2-yl-2-[(3S)-4-cyano-3-[(7-cyanoisoquinolin-1- |
| | yl)amino]butanamido]-4-methyl-1,3-thiazole-5-carboxylate |
| 209 | Propan-2-yl-2-[(3R)-4-cyano-3-[(7-cyanoisoquinolin-1- |
| | yl)amino]butanamido]-4-methyl-1,3-thiazole-5-carboxylate |
| 210 | Propan-2-yl-2-[(3S)-4-cyano-3-{[7-(1-methyl-1H-pyrazol-4- |
| | yl)isoquinolin-1-yl]amino}butanamido]-4-methyl-1,3-thiazole-5- |
| | carboxylate |
| 211 | Tertbutyl-2-[(3S)-4-cyano-3-{[6-(1-methyl-1H-pyrazol-4-yl)pyridin- |
| | 2-yl]formamido}butanamido]-4-methyl-1,3-thiazole-5-carboxylate |
| 212 | Tertbutyl-2-[(3R)-4-cyano-3-{[6-(1-methyl-1H-pyrazol-4-yl)pyridin- |
| | 2-yl]formamido} butanamido]-4-methyl-1,3-thiazole-5-carboxylate |
| 213 | Propan-2-yl-2-[(3S)-4-cyano-3-{[6-(1-methyl-1H-pyrazol-4- |
| | yl)pyridin-2-yl]formamido}butanamido]-4-methyl-1,3-thiazole-5- |
| | carboxylate |
| 214 | Propan-2-yl-2-[(3R)-4-cyano-3-{[6-(1-methyl-1H-pyrazol-4- |
| | yl)pyridin-2-yl]formamido}butanamido]-4-methyl-1,3-thiazole-5- |
| | carboxylate |
| 215 | Ethyl-4-methyl-2-(3-{[3-(5-methyl-1,2,4-oxadiazol-3- |
| | yl)phenyl]formamido}pentanamido)-1,3-thiazole-5-carboxylate |
| 216 | (3R)-4-{[5-(methoxycarbonyl)-4-methyl-1,3-thiazol-2- |
| | yl]carbamoyl}-3-{[3-(5-methyl-1,2,4-oxadiazol-3- |
| | yl)phenyl]formamido}butanoic |
| 217 | (3S)-4-{[5-(methoxycarbonyl)-4-methyl-1,3-thiazol-2- |
| | yl](methyl)carbamoyl}-3-{[3-(5-methyl-1,2,4-oxadiazol-3- |
| | yl)phenyl]formamido}butanoic |
| 218 | Tertbutyl-2-((1s,3s)-N-ethyl-3-((7-(5-methyl-1,2,4-oxadiazol-3- |
| | yl)isoquinolin-1-yl)amino)cyclobutanecarboxamido)-4- |
| | methylthiazole-5-carboxylate |
| 219 | Ethyl-4-methyl-2-(N-methyl-3-(3-(5-methyl-1,2,4-oxadiazol-3- |
| | yl)benzamido)propanamido)thiazole-5-carboxylate |
| 220 | Propyl-4-methyl-2-(((1s,3s)-3-((7-(5-methyl-1,2,4-oxadiazol-3- |
| | yl)isoquinolin-1-yl)amino)cyclobutyl)carbamoyl)thiazole-5- |
| | carboxylate |
| 221 | (1r,3r)-N-(5-(tert-butyl)-4-methylthiazol-2-yl)-1-hydroxy-3-((7-(5- |
| | methyl-1,2,4-oxadiazol-3-yl)isoquinolin-1-yl)amino)cyclobutane-1- |
| | carboxamide |
| 222 | Tertbutyl-2-((1s,3s)-1-fluoro-3-((7-(5-methyl-1,2,4-oxadiazol-3- |
| | yl)isoquinolin-1-yl)amino)cyclobutane-1-carboxamido)-4- |
| | methylthiazole-5-carboxylate |
| 223 | Tertbutyl-2-((1r,3r)-1-fluoro-3-((7-(5-methyl-1,2,4-oxadiazol-3- |
| | yl)isoquinolin-1-yl)amino)cyclobutane-1-carboxamido)-4- |
| | methylthiazole-5-carboxylate |
| 224 | Tertbutyl-4-methyl-2-((1s,3s)-1-methyl-3-((7-(5-methyl-1,2,4- |
| | oxadiazol-3-yl)isoquinolin-1-yl)amino)cyclobutane-1- |
| | carboxamido)thiazole-5-carboxylate |
| 225 | Tertbutyl-4-methyl-2-[(3R)-3-[7-(5-methyl-1,2,4-oxadiazol-3-yl)-1- |
| | isoquinolyl]amino]pyrrolidine-1-carbonyl]amino]thiazole-5- |
| | carboxylate |
| 226 | Tertbutyl-4-methyl-2-[(3S)-3-[7-(5-methyl-1,2,4-oxadiazol-3-yl)-1- |
| | isoquinolyl]amino]pyrrolidine-1-carbonyl]amino]thiazole-5- |
| | carboxylate |
| 227 | Tertbutyl-4-methyl-2-[methyl-[(3S)-3-[7-(5-methyl-1,2,4-oxadiazol- |
| | 3-yl)-1-isoquinolyl]amino]pyrrolidine-1-carbonyl]amino]thiazole-5- |
| | carboxylate |
| 228 | Tertbutyl-4-methyl-2-[3-[3-(5-methyl-1,2,4-oxadiazol-3- |
| | yl)benzoyl]amino]azetidine-1-carbonyl]amino]thiazole-5- |
| | carboxylate |
| 229 | Tertbutyl-4-methyl-2-[methyl-[3-[3-(5-methyl-1,2,4-oxadiazol-3- |
| | yl)benzoyl]amino]azetidine-1-carbonyl]amino]thiazole-5- |
| | carboxylate |
| 230 | Tertbutyl-4-methyl-2-[3-[7-(5-methyl-1,2,4-oxadiazol-3-yl)-1- |
| | isoquinolyl]amino]azetidine-1-carbonyl]amino]thiazole-5- |
| | carboxylate |
| 231 | Tertbutyl-4-methyl-2-[methyl-[3-[7-(5-methyl-1,2,4-oxadiazol-3-yl)- |
| | 1-isoquinolyl]amino]azetidine-1-carbonyl]amino]thiazole-5- |
| | carboxylate |
| 232 | N-(5-(tert-butyl)-4-methylthiazol-2-yl)-3-((7-(5-methyl-1,2,4- |
| | oxadiazol-3-yl)isoquinolin-1-yl)amino)azetidine-1-carboxamide |
| 233 | Propyl-2-(3-(3-chloro-5-(5-methyl-1,2,4-oxadiazol-3- |
| | yl)benzamido)-N-methylpropanamido)-4-methylthiazole-5- |
| | carboxylate |
| 234 | Propyl-4-methyl-2-((2S,4S)-1-methyl-4-(3-(5-methyl-1,2,4- |
| | oxadiazol-3-yl)benzamido)pyrrolidine-2-carboxamido)thiazole-5- |
| | carboxylate. |
| 235 | Propyl-4-methyl-2-((2R,4R)-1-methyl-4-(3-(5-methyl-1,2,4- |
| | oxadiazol-3-yl)benzamido)pyrrolidine-2-carboxamido)thiazole-5- |
| | carboxylate |
| 236 | (2S,4S)-N-(5-(tert-butyl)-4-methylthiazol-2-yl)-1-methyl-4-((7-(5- |
| | methyl-1,2,4-oxadiazol-3-yl)isoquinolin-1-yl)amino)pyrrolidine-2- |
| | carboxamide |
| 237 | Propyl-4-methyl-2-((2S,4S)-1-methyl-4-((7-(5-methyl-1,2,4- |
| | oxadiazol-3-yl)isoquinolin-1-yl)amino)pyrrolidine-2- |
| | carboxamido)thiazole-5-carboxylate |
| 238 | (2S,4S)-1-methyl-4-((7-(5-methyl-1,2,4-oxadiazol-3-yl)isoquinolin- |
| | 1-yl)amino)-N-(1-methyl-5-pentyl-1H-pyrazol-3-yl)pyrrolidine-2- |
| | carboxamide |
| 239 | Propyl-4-methyl-2-((2S,4S)-4-((7-(5-methyl-1,2,4-oxadiazol-3- |
| | yl)isoquinolin-1-yl)amino)pyrrolidine-2-carboxamido)thiazole-5- |
| | carboxylate |
| 240 | Propyl-(S)-4-methyl-2-(3-(3-(5-methyl-1,2,4-oxadiazol-3- |
| | yl)benzamido)pyrrolidine-1-carbonyl)thiazole-5-carboxylate |
| 241 | Propyl-(R)-4-methyl-2-(3-(3-(5-methyl-1,2,4-oxadiazol-3- |
| | yl)benzamido)pyrrolidine-1-carbonyl)thiazole-5-carboxylate |
| 242 | Propyl-4-methyl-2-[(3S)-3-[3-(2-methyltetrazol-5- |
| | yl)benzoyl]amino]pyrrolidine-1-carbonyl]thiazole-5-carboxylate |
| 243 | Propyl-(S)-4-methyl-2-(3-((7-(5-methyl-1,2,4-oxadiazol-3- |
| | yl)isoquinolin-1-yl)amino)pyrrolidine-1-carbonyl)thiazole-5- |
| | carboxylate |
| 244 | Propyl-(R)-4-methyl-2-(3-((7-(5-methyl-1,2,4-oxadiazol-3- |
| | yl)isoquinolin-1-yl)amino)pyrrolidine-1-carbonyl)thiazole-5- |
| | carboxylate |
| 245 | Propyl-(S)-1-methyl-3-(3-((7-(5-methyl-1,2,4-oxadiazol-3- |
| | yl)isoquinolin-1-yl)amino)pyrrolidine-1-carbonyl)-1H-pyrazole-5- |
| | carboxylate |
| 246 | (S)-(5-(tert-butyl)-4-methylthiazol-2-yl)(3-((7-(5-methyl-1,2,4- |
| | oxadiazol-3-yl)isoquinolin-1-yl)amino)pyrrolidin-1-yl)methanone |
| 247 | (S)-(5-cyclopropyl-1-methyl-1H-pyrazol-3-yl)(3-((7-(5-methyl-1,2,4- |
| | oxadiazol-3-yl)isoquinolin-1-yl)amino)pyrrolidin-1-yl)methanone |
| 248 | (S)-(3-((7-(5-methyl-1,2,4-oxadiazol-3-yl)isoquinolin-1- |
| | yl)amino)pyrrolidin-1-yl)(1-methyl-5-phenyl-1H-pyrazol-3- |
| | yl)methanone |
| 249 | (S)-(5-isopropyl-1-methyl-1H-pyrazol-3-yl)(3-((7-(5-methyl-1,2,4- |
| | oxadiazol-3-yl)isoquinolin-1-yl)amino)pyrrolidin-1-yl)methanone |
| 250 | S)-(1H-imidazol-5-yl)(3-((7-(5-methyl-1,2,4-oxadiazol-3- |
| | yl)isoquinolin-1-yl)amino)pyrrolidin-1-yl)methanone |
| 251 | (5-methylisoxazol-3-yl)-[(3S)-3-[[7-(5-methyl-1,2,4-oxadiazol-3-yl)- |
| | 1-isoquinolyl]amino]pyrrolidin-1-yl]methanone |
| 252 | (1-Tert-butylpyrazol-4-yl)-[(3S)-3-[[7-(5-methyl-1,2,4-oxadiazol-3- |
| | yl)-1-isoquinolyl]amino]pyrrolidin-1-yl]methanone |
| 253 | (S)-(5-isopropyl-1H-pyrazol-3-yl)(3-((7-(5-methyl-1,2,4-oxadiazol- |
| | 3-yl)isoquinolin-1-yl)amino)pyrrolidin-1-yl)methanone |
| 254 | (S)-(5-(tert-butyl)-1H-pyrazol-3-yl)(3-((7-(5-methyl-1,2,4-oxadiazol- |
| | 3-yl)isoquinolin-1-yl)amino)pyrrolidin-1-yl)methanone |
| 255 | Propyl-4-methyl-2-((2S,3R)-2-methyl-3-((7-(5-methyl-1,2,4- |
| | oxadiazol-3-yl)isoquinolin-1-yl)amino)pyrrolidine-1- |
| | carbonyl)thiazole-5-carboxylate |
| 256 | Tertbutyl-2-((3R,4R)-3-fluoro-4-((7-(5-methyl-1,2,4-oxadiazol-3- |
| | yl)isoquinolin-1-yl)amino)pyrrolidine-1-carbonyl)-4-methylthiazole- |
| | 5-carboxylate |
| 257 | Propyl-2-methyl-4-(3-{[7-(5-methyl-1,2,4-oxadiazol-3- |
| | yl)isoquinolin-1-yl]amino}propanamido)-1H-imidazole-1- |
| | carboxylate |
| 258 | Propan-2-yl-2-methyl-4-(3-{[7-(5-methyl-1,2,4-oxadiazol-3- |
| | yl)isoquinolin-1-yl]amino}propanamido)-1H-imidazole-1- |
| | carboxylate |
| 259 | Ethyl-2-methyl-4-(3-{[7-(5-methyl-1,2,4-oxadiazol-3-yl)isoquinolin- |
| | 1-yl]amino}propanamido)-1H-imidazole-1-carboxylate |
| 260 | Propyl-4-(3-{[7-(5-methyl-1,2,4-oxadiazol-3-yl)isoquinolin-1- |
| | yl]amino}propanamido)-1H-imidazole-1-carboxylate |
| 261 | Ethyl-2-methyl-4-(3-{[7-(1-methyl-1H-pyrazol-4-yl)isoquinolin-1- |
| | yl]amino}propanamido)-1H-imidazole-1-carboxylate |
| 262 | Propyl-2-methyl-4-(3-{[7-(1-methyl-1H-pyrazol-4-yl)isoquinolin-1- |
| | yl]amino}propanamido)-1H-imidazole-1-carboxylate |
| 263 | Propyl-2-methyl-4-(3-{[3-(5-methyl-1,2,4-oxadiazol-3- |
| | yl)phenyl]formamido}propanamido)-1H-imidazole-1-carboxylate |
| 264 | Propan-2-yl-2-methyl-4-(3-{[3-(5-methyl-1,2,4-oxadiazol-3- |
| | yl)phenyl]formamido}propanamido)-1H-imidazole-1-carboxylate |
| 265 | Ethyl-2-methyl-4-(3-{[3-(5-methyl-1,2,4-oxadiazol-3- |
| | yl)phenyl]formamido}propanamido)-1H-imidazole-1-carboxylate |
| 266 | Propyl-4-(3-{[3-(5-methyl-1,2,4-oxadiazol-3- |
| | yl)phenyl]formamido}propanamido)-1H-imidazole-1-carboxylate |
| 267 | Propyl-4-[(2S)-2-hydroxy-3-{[3-(1-methyl-1H-pyrazol-4- |
| | yl)phenyl]formamido}propanamido]-2-methyl-1H-imidazole-1- |
| | carboxylate |
| 268 | Propyl-4-[(2R)-2-hydroxy-3-{[3-(1-methyl-1H-pyrazol-4- |
| | yl)phenyl]formamido}propanamido]-2-methyl-1H-imidazole-1- |
| | carboxylate |
| 269 | Propan-2-yl-4-[(2S)-2-hydroxy-3-{[3-(1-methyl-1H-pyrazol-4- |
| | yl)phenyl]formamido}propanamido]-2-methyl-1H-imidazole-1- |
| | carboxylate |
| 270 | Propan-2-yl4-[(2R)-2-hydroxy-3-{[3-(1-methyl-1H-pyrazol-4- |
| | yl)phenyl]formamido}propanamido]-2-methyl-1H-imidazole-1- |
| | carboxylate |
| 271 | Ethyl-4-[(2S)-2-hydroxy-3-{[3-(1-methyl-1H-pyrazol-4- |
| | yl)phenyl]formamido}propanamido]-2-methyl-1H-imidazole-1- |
| | carboxylate |
| 272 | Tertbutyl-4-(3-{[7-(5-methyl-1,2,4-oxadiazol-3-yl)isoquinolin-1- |
| | yl]amino} propanamido)-1H-pyrazole-1-carboxylate |
| 273 | Propyl-4-(3-{[7-(5-methyl-1,2,4-oxadiazol-3-yl)isoquinolin-1- |
| | yl]amino}propanamido)-1H-pyrazole-1-carboxylate |
| 274 | Tertbutyl-4-(3-{[3-(5-methyl-1,2,4-oxadiazol-3- |
| | yl)phenyl]formamido}propanamido)-1H-pyrazole-1-carboxylate |
| 275 | Propyl-4-(3-{[3-(5-methyl-1,2,4-oxadiazol-3- |
| | yl)phenyl]formamido}propanamido)-1H-pyrazole-1-carboxylate |
| 276 | Propyl-4-methyl-2-(3-{[6-(5-methyl-1,2,4-oxadiazol-3-yl)pyridin-2- |
| | yl]formamido}propanamido)-1,3-thiazole-5-carboxylate |
| 277 | Tertbuyl-4-methyl-2-(3-{[6-(5-methyl-1,2,4-oxadiazol-3-yl)pyridin- |
| | 2-yl]formamido}propanamido)-1,3-thiazole-5-carboxylate |
| 278 | Propan-2-yl-4-methyl-2-(3-{[6-(5-methyl-1,2,4-oxadiazol-3- |
| | yl)pyridin-2-yl]formamido}propanamido)-1,3-thiazole-5-carboxylate |
| 279 | Tertbutyl-2-(3-{[2-fluoro-3-(5-methyl-1,2,4-oxadiazol-3- |
| | yl)phenyl]formamido}propanamido)-4-methyl-1,3-thiazole-5- |
| | carboxylate |
| 280 | Propyl-4-methyl-2-[3-[(7-methyl-1-isoquinolyl)amino]propanoyl- |
| | amino]thiazole-5-carboxylate |

and physiologically acceptable salts, derivatives, solvates, prodrugs and stereoisomers thereof, including mixtures thereof in all ratios.

Furthermore, the abbreviations below have the following meanings:
Boc ter-butoxycarbonyl
CBZ benzyloxycarbonyl
DNP 2,4-dinitrophenyl
FMOC 9-fluorenylmethoxycarbonyl
imi-DNP 2,4-dinitrophenyl in the 1-position of the imidazole ring
OMe methyl ester
POA phenoxyacetyl
DCCI dicyclohexylcarbodiimide
HOBt 1-hydroxybenzotriazole

The invention further relates to a pharmaceutical preparation comprising one or more compounds according to the present invention and/or one of its physiologically acceptable salts, derivatives, solvates, prodrugs and stereoisomers, including mixtures thereof in all ratios.

The invention also relates to a pharmaceutical preparation according to the invention of this type, comprising further excipients and/or adjuvants.

In addition, the invention relates to an above pharmaceutical preparation according to the invention, comprising at least one further medicament active compound.

Pharmaceutically or physiologically acceptable derivatives are taken to mean, for example, salts of the compounds of the present invention, and also so-called pro-drug compounds. Prodrug compounds are taken to mean derivatives of the compounds of the present invention which have been modified by means of, for example, alkyl or acyl groups (see also amino- and hydroxyl-protecting groups below), sugars or oligopeptides and which are rapidly cleaved or liberated in the organism to form the effective molecules. These also include biodegradable polymer derivatives of the compound of the present invention, as described, for example, in Int. J. Pharm. 115 (1995), 61-67.

The compound of the present invention can be used in its final non-salt form. On the other hand, the present invention also encompasses the use of the compound of the present invention in the form of its pharmaceutically acceptable salts, which can be derived from various organic and inorganic bases by procedures known in the art. Pharmaceutically acceptable salt forms of the compound of the present invention are for the most part prepared by conventional methods. If the compound of the present invention contains a carboxyl group, one of its suitable salts can be formed by reacting the compound of the present invention ith a suitable base to give the corresponding base-addition salt. Such bases are, for example, alkali metal hydroxides, including potassium hydroxide, sodium hydroxide and lithium hydroxide; alkaline-earth metal hydroxides, such as barium hydroxide and calcium hydroxide; alkali metal alkoxides, for example potassium ethoxide and sodium propoxide; and various organic bases, such as piperidine, diethanolamine and N-methylglutamine. The aluminium salts of the compound of the present invetion are likewise included. Furthermore, the base salts of the compounds of the present invention include aluminium, ammonium, calcium, copper, iron(III), iron(II), lithium, magnesium, man-ganese(III), manganese(II), potassium, sodium and zinc salts, but this is not intended to represent a restriction.

Of the above-mentioned salts, preference is given to ammonium; the alkali metal salts sodium and potassium, and the alkaline-earth metal salts calcium and magnesium. Salts of the compounds of the present invention which are derived from pharmaceutically acceptable organic non-toxic bases include salts of primary, secondary and tertiary amines, substituted amines, also including naturally occurring substituted amines, cyclic amines, and basic ion exchanger resins, for example arginine, betaine, caffeine, chloroprocaine, choline, N,N′-dibenzylethylen-ediamine (benzathine), dicyclohexylamine, diethanolamine, diethylamine, 2-diethyl-aminoethanol, 2-dimethylaminoethanol, ethanolamine, ethylenediamine, N-ethylmorpholine, N-ethylpiperidine, glucamine, glucosamine, histidine, hydrabamine, isopropylamine, lidocaine, lysine, meglumine, N-methyl-D-glucamine, morpholine, piperazine, piperidine, polyamine resins, procaine, purines, theobromine, triethanolamine, triethylamine, trimethylamine, tripropylamine and tris-(hydroxymethyl)methylamine (tromethamine), but this is not intended to represent a restriction.

As mentioned, the pharmaceutically acceptable base-addition salts of the compound of the present invention are formed with metals or amines, such as alkali metals and alkaline-earth metals or organic amines. Preferred metals are sodium, potassium, magnesium and calcium. Preferred organic amines are N,N′-dibenzylethylenediamine, chloroprocaine, choline, diethanolamine, ethylene-diamine, N-methyl-D-glucamine and procaine.

The base-addition salts of the compounds of the present invention are prepared by bringing the free acid form into contact with a sufficient amount of the desired base, causing the formation of the salt in a conventional manner. The free acid can be regenerated by bringing the salt form into contact with an acid and isolating the free acid in a conventional manner. The free acid forms differ in a certain respect from the corresponding salt forms thereof with respect to certain physical properties, such as solubility in polar solvents; for the purposes of the invention, however, the salts otherwise correspond to the respective free acid forms thereof.

In view of that stated above, it can be seen that the term “pharmaceutically acceptable salt” in the present connection is taken to mean an active compound which comprises the compound of the present invention in the form of one of its salts, in particular if this salt form imparts improved pharmacokinetic properties on the active compound compared with the free form of the active compound or any other salt form of the active compound used earlier. The pharmaceutically acceptable salt form of the active compound can also provide this active compound for the first time with a desired pharmacokinetic property which it did not have earlier and can even have a positive influence on the pharmacodynamics of this active compound with respect to its therapeutic efficacy in the body.

Solvates of the compound of the present invention are taken to mean adductions of inert solvent molecules of the compound of the present invention which form owing to their mutual attractive force. Solvates are, for example, hydrates, such as monohydrates or dihydrates, or alcoholates, i.e. addition compounds with alcohols, such as, for example, with methanol or ethanol.

All physiologically acceptable salts, derivatives, solvates and stereoisomers of these compounds, including mixtures thereof in all ratios, are also in accordance with the invention.

Compounds of the present invention may contain one or more centres of chirality, so that all stereoisomers, enentiomers, diastereomers, etc., of the compounds of the present inventionare also claimed in the present invention.

The invention also relates to the optically active forms (stereoisomers), the enantiomers, the racemates, the diastereomers and hydrates and solvates of these compounds.

Compounds of the present invention according to the invention may be chiral owing to their molecular structure and may accordingly occur in various enantiomeric forms. They may therefore be in racemic or optically active form. Since the pharmaceutical efficacy of the racemates or stereoisomers of the compounds according to the invention may differ, it may be desirable to use the enantiomers. In these cases, the end product, but also even the intermediates, may be separated into enantiomeric compounds by chemical or physical measures known to the person skilled in the art or already employed as such in the synthesis.

Pharmaceutically or physiologically acceptable derivatives are taken to mean, for example, salts of the compounds according to the invention and also so-called prodrug compounds. Prodrug compounds are taken to mean compounds of the present invention which have been modified with, for example, alkyl or acyl groups (see also amino- and hydroxyl-protecting groups below), sugars or oligopeptides and which are rapidly cleaved or liberated in the organism to form the effective compounds according to the invention. These also include biodegradable polymer derivatives of the compounds according to the invention, as described, for example, in Int. J. Pharm. 115 (1995), 61-67.

Suitable acid-addition salts are inorganic or organic salts of all physiologically or pharmacologically acceptable acids, for example halides, in particular hydrochlorides or hydrobromides, lactates, sulfates, citrates, tartrates, maleates, fumarates, oxalates, acetates, phosphates, methylsulfonates or p-toluenesulfonates.

Very particular preference is given to the hydrochlorides, the trifluoroacetates or the bistrifluoroacetates of the compounds according to the invention.

Solvates of the compounds of the present invention are taken to mean adductions of inert solvent molecules onto the compounds of the present invention which form owing to their mutual attractive force. Solvates are, for example, hydrates, such as monohydrates or dihydrates, or alcoholates, i.e. addition compounds with alcohols, such as, for example, with methanol or ethanol.

It is furthermore intended that a compound of the present invention includes iso-tope-labelled forms thereof. An isotope-labelled form of a compound of the present inventionis identical to this compound apart from the fact that one or more atoms of the compound have been replaced by an atom or atoms having an atomic mass or mass number which differs from the atomic mass or mass number of the atom which usually occurs naturally. Examples of isotopes which are readily commercially available, and which can be incorporated into a compound of the present inventionby well-known methods include isotopes of hydrogen, carbon, nitrogen, oxygen, phosphorus, fluorine and chlorine, for example ²H, ³H, ¹³C ¹⁴C¹⁵N,¹⁸O,¹⁷O, ³¹P, ³²P, ³⁵S, ¹⁸F and ³⁶Cl, respectively. A compound of the present invention, a prodrug thereof or a pharmaceutically acceptable salt of either which contains one or more of the above-mentioned isotopes and/or other isotopes of other atoms is intended to be part of the present invention. An isotope-labelled compound of the present invention can be used in a number of beneficial ways. For example, an isotope-labelled compound of the present invention into which, for example, a radioisotope, such as ³H or ¹⁴C, has been incorporated is suitable for medicament and/or substrate tissue distribution assays. These radioisotopes, i.e. tritium (³H) and carbon-14 (¹⁴C), are particularly preferred owing to their simple preparation and excellent detectability. Incorporation of heavier isotopes, for example deuterium (²H), into a compound of the present invention has therapeutic advantages owing to the higher metabolic stability of this isotope-labelled compound. Higher metabolic stability translates directly into an increased in-vivo half-life or lower dosages, which under most circumstances would represent a preferred embodiment of the present invention. An isotope-labelled compound of the present invention can usually be prepared by carrying out the procedures disclosed in the synthesis schemes and the related description, in the example part and in the preparation part in the present text, replacing a non-isotope-labelled reactant with a readily available isotope-labelled reactant.

In order to manipulate the oxidative metabolism of the compound by way of the primary kinetic isotope effect, deuterium (²H) can also be incorporated into a com-pound of the present invention. The primary kinetic isotope effect is a change in the rate of a chemical reaction that results from exchange of isotopic nuclei, which in turn is caused by the change in ground state energies necessary for covalent bond formation after this isotopic exchange. Exchange of a heavier isotope usually results in a lowering of the ground state energy for a chemical bond and thus causes a reduction in the rate in rate-limiting bond breakage. If the bond breakage occurs in or in the vicinity of a saddle-point region along the coordinate of a multi-product reaction, the product distribution ratios can be altered substantially. For explanation: if deuterium is bonded to a carbon atom in a non-exchangeable position, rate differences of k_{M}/k_{D}=2-7 are typical. If this rate difference is successfully applied to a compound of the present invention that is susceptible to oxidation, the profile of this compound in vivo can thereby be drastically modified and result in improved pharmacokinetic properties.

When discovering and developing therapeutic agents, the person skilled in the art attempts to optimise pharmacokinetic parameters while retaining desirable in-vitro properties. It is reasonable to assume that many compounds with poor pharma-cokinetic profiles are susceptible to oxidative metabolism. In-vitro liver microsomal assays currently available provide valuable information on the course of oxidative metabolism of this type, which in turn permits the rational design of deuterated compounds of the present invention with improved stability through resistance to such oxidative metabolism. Significant improvements in the pharmacokinetic profiles of the compounds of the present invention are thereby obtained and can be expressed quantitatively in terms of increases in the in-vivo half-life (T/2), concen-tration at maximum therapeutic effect (Cmaₓ), area under the dose response curve (AUC), and F; and in terms of reduced clearance, dose and costs of materials.

The following is intended to illustrate the above: a compound of the present invention which has multiple potential sites of attack for oxidative metabolism, for example benzylic hydrogen atoms and hydrogen atoms bonded to a nitrogen atom, is prepared as a series of analogues in which various combinations of hydrogen atoms are replaced by deuterium atoms, so that some, most or all of these hydrogen atoms have been replaced by deuterium atoms. Half-life determinations enable favourable and accurate determination of the extent to which the improve-ment in resistance to oxidative metabolism has improved. In this way, it is determined that the half-life of the parent compound can be extended by up to 100% as the result of deuterium-hydrogen exchange of this type.

The replacement of hydrogen by deuterium in a compound of the present inventioncan also be used to achieve a favourable modification of the metabolite spectrum of the starting compound in order to diminish or eliminate undesired toxic metabolites. For example, if a toxic metabolite arises through oxidative carbon-hydrogen (C-H) bond cleavage, it can reasonably be assumed that the deuterated analogue will greatly diminish or eliminate production of the undesired metabolite, even if the particular oxidation is not a rate-determining step. Further information on the state of the art with respect to deuterium-hydrogen exchange is given, for example in Hanzlik et al., J. Org. Chem. 55, 3992-3997, 1990, Reider et al., J. Org. Chem. 52, 3326-3334, 1987, Foster, Adv. Drug Res. 14, 1-40, 1985, Gillette et al., Biochemistry 33(10), 2927-2937, 1994, and Jarman et al., Carcinogenesis 16(4), 683-688, 1993.

The invention also relates to mixtures of the compounds of the present invention according to the invention, for example mixtures of two diastereomers, for example in the ratio 1:1, 1:2, 1:3, 1:4, 1:5, 1:10, 1:100 or 1:1000. These are particularly preferably mixtures of two stereoisomeric compounds. However, preference is also given to mixtures of two or more compounds of the present invention.

In addition, the invention relates to a process for the preparation of the compounds of the present invention, characterized in that
a) the base of a compound of the present invention is converted into one of its salts by treatment with an acid, or
b) an acid of a compound of the present invention is converted into one of its salts by treatment with a base.

It is also possible to carry out the reactions stepwise in each case and to modify the sequence of the linking reactions of the building blocks with adaptation of the protecting-group concept.

The starting materials or starting compounds are generally known. If they are novel, they can be prepared by methods known per se.

If desired, the starting materials can also be formed in situ by not isolating them from the reaction mixture, but instead immediately converting them further into the compounds of the present invention.

The compounds of the present invention are preferably obtained by liberating them from their functional derivatives by solvolysis, in particular by hydrolysis, or by hydrogenolysis. Preferred starting materials for the solvolysis or hydrogenolysis are those which contain correspondingly protected amino, carboxyl and/or hydroxyl groups instead of one or more free amino, carboxyl and/or hydroxyl groups, preferably those which carry an amino-protecting group instead of an H atom which is connected to an N atom. Preference is furthermore given to starting materials which carry a hydroxyl-protecting group instead of the H atom of a hydroxyl group.

Preference is also given to starting materials which carry a protected carboxyl group instead of a free carboxyl group. It is also possible for a plurality of identical or different protected amino, carboxyl and/or hydroxyl groups to be present in the molecule of the starting material. If the protecting groups present are different from one another, they can in many cases be cleaved off selectively.

The term “amino-protecting group” is generally known and relates to groups which are suitable for protecting (blocking) an amino group against chemical reactions, but which can easily be removed after the desired chemical reaction has been carried out elsewhere in the molecule. Typical of such groups are, in particular, unsubstituted or substituted acyl groups, furthermore unsubstituted or substituted aryl (for example 2,4-dinitophenyl) or aralkyl groups (for example benzyl, 4-nitrobenzyl, triphenylmethyl). Since the amino-protecting groups are removed after the desired reaction or reaction sequence, their type and size are, in addition, not crucial, but preference is given to those having 1-20, in particular 1-8, C atoms. The term “acyl group” is to be understood in the broadest sense in connection with the present process. It encompasses acyl groups derived from aliphatic, araliphatic, aromatic or heterocyclic carboxylic acids or sulfonic acids and, in particular, alkoxy-carbonyl, aryloxycarbonyl and especially aralkoxycarbonyl groups. Examples of such acyl groups are alkanoyl, such as acteyl, propionyl, buturyl, aralkanoyl, such as phenylacetyl, aroyl, such as benzoyl or toluyl, aryoxyaklkanoyl, such as phenoxyacetyl, alkyoxycarbonyyl, such as methoxycarbonyl, ethoxycarbonyl, 2,2,2-trichloroethoxycarbonyl, BOC, 2-iodoethoxycaronyl, aralkoxycarbonyl, such as CBZ, 4-methoxybenzyloxycarbonyl or FMOC. Preferred acyl groups are CBZ, FMOC, benzyl and acetyl.

The term “acid-protecting group” or “carboxyl-protecting group” is likewise generally known and relates to groups which are suitable for protecting a —COOH group against chemical reactions, but which can easily be removed after the desired chemical reaction has been carried out elsewhere in the molecule. The use of esters instead of the free acids, for example of substituted and unsubstituted alkyl esters (such as methyl, ethyl, tert-butyl and substituted derivatives thereof), of substituted and unsubstituted benzyl esters or silyl esters, is typical. The type and size of the acid-protecting groups is not crucial, but preference is given to those having 1-20, in particular 1-10, C atoms.

The term “hydroxyl-protecting group” is likewise generally known and relates to groups which are suitable for protecting a hydroxyl group against chemical reactions, but which can easily be removed after the desired chemical reaction has been carried out elsewhere in the molecule. Typical of such groups are the above-mentioned unsubstituted or substituted aryl, aralkyl or acyl groups, furthermore also alkyl groups. Their type and size of the hydroxyl-protecting groups is not crucial, but preference is given to those having 1-20, in particular 1-10, C atoms. Examples of hyrdoxyl-protecting groups are, inter alia, benzyl, p-nitrobenzoyl, p-toluenesulfonyl and acetyl, where benzyl and acetyl are preferred.

Further typical examples of amino-, acid- and hydroxyl-protecting groups are found, for example, in “Greene's Protective Groups in Organic Synthesis”, fourth edition, Wiley-Interscience, 2007.

The functional derivatives of the compounds of the present invention to be used as starting materials can be prepared by known methods of amino-acid and peptide synthesis, as described, for example, in the said standard works and patent applications.

The compounds of the present invention are liberated from their functional deriva-tives, depending on the protecting group used, for example, with the aid of strong acids, advantageously using trifluoroacetic acid or perchloric acid, but also using other strong inorganic acids, such as hydrochloric acid or sulfuric acid, strong organic acids, such as trichloroacetic acid, or sulfonic acids, such as benzoyl- or p-toluenesulfonic acid. The presence of an additional inert solvent and/or a catalyst is possible but is not always necessary.

Depending on the respective synthetic route, the starting materials can optionally be reacted in the presence of an inert solvent.

Suitable inert solvents are, for example, heptane, hexane, petroleum ether, DMSO, benzene, toluene, xylene, trichloroethylene-, 1,2-dichloroethane, carbon tetrachloride, chloroform or dichloromethane; alcohols, such as methanol, ethanol, isopropanol, n-propanol, n-butanol or tert-butanol; ethers, such as diethyl ether, diisopropyl ether (preferably for substitution on the indole nitrogen), tetrahydrofuran (THF) or dioxane; glycol ethers, such as ethylene glycol monomethyl or monoethyl ether, ethylene glycol dimethyl ether (diglyme); ketones, such as acetone or butanone; amides, such as acetamide, dimethylacetamide, N-methylpyrrolidone (NMP) or dimethylformamide (DMF); nitriles, such as acetonitrile; esters, such as ethyl acetate, carboxylic acids or acid anhydrides, such as, for example, such as acetic acid or acetic anhydride, nitro compounds, such as nitromethane or nitro-benzene, optionally also mixtures of the said solvents with one another or mixtures with water.

The amount of solvent is not crucial; 10 g to 500 g of solvent can preferably be added per g of the compound of the present invention to be reacted.

It may be advantageous to add an acid-binding agent, for example an alkali metal or alkaline-earth metal hydroxide, carbonate or bicarbonate or other alkali or alkaline-earth metal salts of weak acids, preferably a potassium, sodium or calcium salt, or to add an organic base, such as, for example, triethylamine, dimethylamine, pyridine or quinoline, or an excess of the amine component.

The resultant compounds according to the invention can be separated from the corresponding solution in which they are prepared (for example by centrifugation and washing) and can be stored in another composition after separation, or they can remain directly in the preparation solution. The resultant compounds according to the invention can also be taken up in desired solvents for the particular use.

The reaction duration depends on the reaction conditions selected. In general, the reaction duration is 0.5 hour to 10 days, preferably 1 to 24 hours. On use of a microwave, the reaction time can be reduced to values of 1 to 60 minutes. The compounds of the present invention and also the starting materials for their preparation are, in addition, prepared by known methods, as described in the literature (for example in standard works, such as Houben-Weyl, Methoden der organischen Chemie [Methods of Organic Chemistry], Georg-Thieme-Verlag, Stuttgart), for example under reaction conditions which are known and suitable for the said reactions. Use can also be made here of variants known per se, which are not described here in greater detail.

Conventional work-up steps, such as, for example, addition of water to the reaction mixture and extraction, enable the compounds to be obtained after removal of the solvent. It may be advantageous, for further purification of the product, to follow this with a distillation or crystallisation or to carry out a chromatographic purification.

An acid of the present invention can be converted into the associated addition salt using a base, for example by reaction of equivalent amounts of the acid and base in an inert solvent, such as ethanol, and inclusive evaporation. Suitable bases for this reaction are, in particular, those which give physiologically acceptable salts. Thus, the acid of the present inventioncan be converted into the corresponding metal salt, in particular alkali or alkaline-earth metal salt, using a base (for example sodium hydroxide, potassium hydroxide, sodium carbonate or potassium carbonate) or into the corresponding ammonium salt. Organic bases which give physiologically acceptable salts, such as, for example, ethanolamine, are also suitable for this reaction.

On the other hand, a base of the present invention can be converted into the asso-ciated acid-addition salt using an acid, for example by reaction of equivalent amounts of the base and acid in an inert solvent, such as ethanol, with subsequent evaporation. Suitable acids for this reaction are, in particular, those which give physiologically acceptable salts. Thus, it is possible to use inorganic acids, for example sulfuric acid, nitric acid, hydrohalic acids, such as hydrochloric acid or hydrobromic acid, phosphoric acids, such as orthophosphoric acid, sulfamic acid, furthermore organic acids, in particular aliphatic, alicyclic, araliphatic, aromatic or heterocyclic, mono- or polybasic carboxylic, sulfonic or sulfuric acids, for example formic acid, acetic acid, propionic acid, pivalic acid, diethylacetic acid, malonic acid, succinic acid, pimelic acid, fumaric acid, maleic acid, lactic acid, tartaric acid, malic acid, citric acid, gluconic acid, ascorbic acid, nicotinic acid, isonicotinic acid, methane- or ethanesulfonic acid, ethanedisulfonic acid, 2-hydroxysulfonic acid, benzenesulfonic acid, p-toluenesulfonic acid, naphthalenemom- and disulfonic acids or laurylsulfuric acid. Salts with physiologically unacceptable acids, for example picrates, can be used for the isolation and/or purification of the compounds of the present invention.

It has been found that the compounds of the present invention are well tolerated and have valuable pharmacological properties.

The invention therefore furthermore relates to the use of compounds according to the invention for the preparation of a medicament for the treatment and/or prophylaxis of diseases which are caused, promoted and/or propagated by HSET.

The invention thus also relates, in particular, to a medicament comprising at least one compound according to the invention and/or one of its physiologically acceptable salts, derivatives, solvates, prodrugs and stereoisomers, including mixtures thereof in all ratios, for use in the treatment and/or prophylaxis of physiological and/or pathophysiological states.

Particular preference is given to physiological and/or pathophysiological states which are connected to HSET.

Physiological and/or pathophysiological states are taken to mean physiological and/or pathophysiological states which are medically relevant, such as, for example, diseases or illnesses and medical disorders, complaints, symptoms or complications and the like, in particular diseases.

The invention furthermore relates to a medicament comprising at least one compound according to the invention and/or one of its physiologically acceptable salts, derivatives, solvates, prodrugs and stereoisomers, including mixtures thereof in all ratios, for use in the treatment and/or prophylaxis of physiological and/or pathophysiological states selected from the group consisting of hyperproliferative diseases and disorders.

The invention further relates to a medicament comprising at least one compound according to the invention and/or one of its physiologically acceptable salts, derivatives, solvates, prodrugs and stereoisomers, including mixtures thereof in all ratios, for use in the treatment and/or prophylaxis of physiological and/or pathophysiological states selected from the group consisting of hyperproliferative and infectious diseases and disorders, wherein the hyperproliferative disease or disorder is cancer.

The invention thus particularly preferably relates to a medicament comprising at least one compound according to the invention and/or one of its physiologically acceptable salts, derivatives, solvates, prodrugs and stereoisomers, including mixtures thereof in all ratios, wherein the cancer is selected from the group consisting of acute and chronic lymphocytic leukemia, acute granulocytic leukemia, adrenal cortex cancer, bladder cancer, brain cancer, breast cancer, cervical hyperplasia, cervical cancer, chorio cancer, chronic granulocytic leukemia, chronic lymphocytic leukemia, colon cancer, endometrial cancer, esophageal cancer, essential thrombocytosis, genitourinary carcinoma, glioma, glioblastoma, hairy cell leukemia, head and neck carcinoma, Hodgkin's disease, Kaposi's sarcoma, lung carcinoma, lymphoma, malignant carcinoid carcinoma, malignant hypercalcemia, malignant melanoma, malignant pancreatic insulinoma, medullary thyroid carcinoma, melanoma, multiple myeloma, mycosis fungoides, myeloid and lymphocytic leukemia, neuroblastoma, non-Hodgkin's lymphoma, non-small cell lung cancer, osteogenic sarcoma, ovarian carcinoma, pancreatic carcinoma, polycythemia vera, primary brain carcinoma, primary macroglobulinemia, prostatic cancer, renal cell cancer, rhabdomyosarcoma, skin cancer, small-cell lung cancer, soft-tissue sarcoma, squamous cell cancer, stomach cancer, testicular cancer, thyroid cancer and Wilms' tumor.

The invention further preferably relates to a medicament comprising at least one compound according to the invention and/or one of its physiologically acceptable salts, derivatives, solvates, prodrugs and stereoisomers, including mixtures thereof in all ratios, for use in the treatment and/or prophylaxis of physiological and/or pathophysiological states selected from the group consisting of hyperproliferative and infectious diseases and disorders, wherein the hyperproliferative disease or disorder is selected from the group consisting of age-related macular degeneration, Crohn's disease, cirrhosis, chronic inflammatory-related disorders, proliferative diabetic retinopathy, proliferative vitreoretinopathy, retinopathy of prematurity, granulomatosis, immune hyperproliferation associated with organ or tissue transplantation and an immunoproliferative disease or disorder selected from the group consisting of inflammatory bowel disease, psoriasis, rheumatoid arthritis, systemic lupus erythematosus (SLE), vascular hyperproliferation secondary to retinal hypoxia and vasculitis.

It is intended that the medicaments disclosed above include a corresponding use of the compounds according to the invention for the preparation of a medicament for the treatment and/or prophylaxis of the above physiological and/or pathophysiological states.

It is additionally intended that the medicaments disclosed above include a corresponding method for the treatment and/or prophylaxis of the above physiological and/or pathophysiological states in which at least one compound according to the invention is administered to a patient in need of such a treatment.

The compounds according to the invention preferably exhibit an advantageous biological activity which can easily be demonstrated in enzyme assays and animal experiments, as described in the examples. In such enzyme-based assays, the compounds according to the invention preferably exhibit and cause an inhibiting effect, which is usually documented by IC₅₀ values in a suitable range, preferably in the micromolar range and more preferably in the nanomolar range.

The compounds according to the invention can be administered to humans or animals, in particular mammals, such as apes, dogs, cats, rats or mice, and can be used in the therapeutic treatment of the human or animal body and in the combating of the above-mentioned diseases. They can furthermore be used as diagnostic agents or as reagents.

Furthermore, compounds according to the invention can be used for the isolation and investigation of the activity or expression of HSET. In addition, they are particularly suitable for use in diagnostic methods for diseases in connection with disturbed HSET activity. The invention therefore furthermore relates to the use of the compounds according to the invention for the isolation and investigation of the activity or expression of HSET or as binders and inhibitors of HSET.

For diagnostic purposes, the compounds according to the invention can, for example, be radioactively labelled. Examples of radioactive labels are ³H, ¹⁴C, ²³¹I, and ¹²⁵I. A preferred labelling method is the iodogen method (Fraker et al., 1978). In addition, the compounds according to the invention can be labelled by enzymes, fluorophores and chemophores. Examples of enzymes are alkaline phosphatase, 3-galactosidase and glucose oxidase, an example of a fluorophore is fluorescein, an example of a chemophore is luminol, and automated detection systems, for example for fluorescent colorations, are described, for example, in U.S. Pat. Nos. 4,125,828 and 4,207,554.

The present invention further relates to pharmaceutical compositions containing the compounds of the present invention and their use for the treatment and/or prophylaxis of diseases and disorders where the partial or total inactivation of HSET could be beneficial.

The compounds of the present invention can be used for the preparation of pharma-ceutical preparations, in particular by non-chemical methods. In this case, they are brought into a suitable dosage form together with at least one solid, liquid and/or semi-liquid excipient or adjuvant and optionally in combination with one or more further active compound(s).

The invention therefore furthermore relates to pharmaceutical preparations comprising at least one compound of the present invention and/or physiologically acceptable salts, derivatives, solvates and stereoisomers thereof, including mixtures thereof in all ratios. In particular, the invention also relates to pharmaceutical preparations which comprise further excipients and/or adjuvants, and also to pharmaceutical preparations which comprise at least one further medicament active compound.

In particular, the invention also relates to a process for the preparation of a pharmaceutical preparation, characterised in that a compound of the present inventionand/or one of its physiologically acceptable salts, derivatives, solvates and stereoisomers, including mixtures thereof in all ratios, is brought into a suitable dosage form together with a solid, liquid or semi-liquid excipient or adjuvant and optionally with a further medicament active compound.

The pharmaceutical preparations according to the invention can be used as medicaments in human or veterinary medicine. The patient or host can belong to any mammal species, for example a primate species, particularly humans; rodents, including mice, rats and hamsters; rabbits; horses, cattle, dogs, cats, etc. Animal models are of interest for experimental investigations, where they provide a model for the treatment of a human disease.

Suitable carrier substances are organic or inorganic substances which are suitable for enteral (for example oral), parenteral or topical administration and do not react with the novel compounds, for example water, vegetable oils (such as sunflower oil or cod-liver oil), benzyl alcohols, polyethylene glycols, gelatine, carbohydrates, such as lactose or starch, magnesium stearate, talc, lanolin or vaseline. Owing to his expert knowledge, the person skilled in the art is familiar which adjuvants are suitable for the desired medicament formulation. Besides solvents, for example water, physiological saline solution or alcohols, such as, for example, ethanol, propanol or glycerol, sugar solutions, such as glucose or mannitol solutions, or a mixture of the said solvents, gel formers, tablet assistants and other active-ingredient carriers, it is also possible to use, for example, lubricants, stabilisers and/or wetting agents, emulsifiers, salts for influencing the osmotic pressure, anti-oxidants, dispersants, antifoams, buffer substances, flavours and/or aromas or flavour correctants, preservatives, solubilisers or dyes. If desired, preparations or medicaments according to the invention may comprise one or more further active compounds, for example one or more vitamins.

If desired, preparations or medicaments according to the invention may comprise one or more further active compounds and/or one or more action enhancers (adjuvants).

The terms “pharmaceutical formulation” and “pharmaceutical preparation” are used as synonyms for the purposes of the present invention.

As used here, “pharmaceutically tolerated” relates to medicaments, precipitation reagents, excipients, adjuvants, stabilisers, solvents and other agents which facilitate the administration of the pharmaceutical preparations obtained therefrom to a mammal without undesired physiological side effects, such as, for example, nausea, dizziness, digestion problems or the like.

In pharmaceutical preparations for parenteral administration, there is a requirement for isotonicity, euhydration and tolerability and safety of the formulation (low toxicity), of the adjuvants employed and of the primary packaging. Surprisingly, the compounds according to the invention preferably have the advantage that direct use is possible and further purification steps for the removal of toxicologically unaccept-able agents, such as, for example, high concentrations of organic solvents or other toxicologically unacceptable adjuvants, are thus unnecessary before use of the compounds according to the invention in pharmaceutical formulations.

The invention particularly preferably also relates to pharmaceutical preparations comprising at least one compound according to the invention in precipitated non-crystalline, precipitated crystalline or in dissolved or suspended form, and optionally excipients and/or adjuvants and/or further pharmaceutical active compounds.

The compounds according to the invention preferably enable the preparation of highly concentrated formulations without unfavourable, undesired aggregation of the compounds according to the invention occurring. Thus, ready-to-use solutions having a high active-ingredient content can be prepared with the aid of compounds according to the invention with aqueous solvents or in aqueous media.

The compounds and/or physiologically acceptable salts and solvates thereof can also be lyophilised and the resultant lyophilisates used, for example, for the preparation of injection preparations.

Aqueous preparations can be prepared by dissolving or suspending compounds according to the invention in an aqueous solution and optionally adding adjuvants.

To this end, defined volumes of stock solutions comprising the said further adjuvants in defined concentration are advantageously added to a solution or suspension having a defined concentration of compounds according to the invention, and the mixture is optionally diluted with water to the pre-calculated concentration. Alternatively, the adjuvants can be added in solid form. The amounts of stock solutions and/or water which are necessary in each case can subsequently be added to the aqueous solution or suspension obtained. Compounds according to the invention can also advantageously be dissolved or suspended directly in a solution comprising all further adjuvants.

The solutions or suspensions comprising compounds according to the invention and having a pH of 4 to 10, preferably having a pH of 5 to 9, and an osmolality of 250 to 350 mosmol/kg can advantageously be prepared. The pharmaceutical preparation can thus be administered directly substantially without pain intravenously, intra-arterially, intraarticularly, subcutaneously or percutaneously. In addition, the preparation may also be added to infusion solutions, such as, for example, glucose solution, isotonic saline solution or Ringer's solution, which may also contain further active compounds, thus also enabling relatively large amounts of active compound to be administered.

Pharmaceutical preparations according to the invention may also comprise mixtures of a plurality of compounds according to the invention.

The preparations according to the invention are physiologically well tolerated, easy to prepare, can be dispensed precisely and are preferably stable with respect to assay, decomposition products and aggregates throughout storage and transport and during multiple freezing and thawing processes. They can preferably be stored in a stable manner over a period of at least three months to two years at refrigerator temperature (2-8° C.) and at rt (23-27° C.) and 60% relative atmospheric humidity (R. H.).

For example, the compounds according to the invention can be stored in a stable manner by drying and when necessary converted into a ready-to-use pharmaceutical preparation by dissolution or suspension. Possible drying methods are, for example, without being restricted to these examples, nitrogen-gas drying, vacuum-oven drying, lyophilisation, washing with organic solvents and subsequent air drying, liquid-bed drying, fluidised-bed drying, spray drying, roller drying, layer drying, air drying at rt and further methods.

The term “effective amount” denotes the amount of a medicament or of a pharmaceutical active compound which causes in a tissue, system, animal or human a biological or medical response which is sought or desired, for example, by a researcher or physician.

In addition, the term “therapeutically effective amount” denotes an amount which, compared with a corresponding subject who has not received this amount, has the following consequence: improved treatment, healing, prevention or elimination of a disease, syndrome, disease state, complaint, disorder or prevention of side effects or also a reduction in the progress of a disease, complaint or disorder. The term “therapeutically effective amount” also encompasses the amounts which are effective for increasing normal physiological function.

On use of preparations or medicaments according to the invention, the compounds according to the invention and/or physiologically acceptable salts and solvates thereof are generally used analogously to known, commercially available preparations or preparations, preferably in dosages of between 0.1 and 500 mg, in particular 5 and 300 mg, per use unit. The daily dose is preferably between 0.001 and 250 mg/kg, in particular 0.01 and 100 mg/kg, of body weight. The preparation can be administered one or more times per day, for example two, three or four times per day. However, the individual dose for a patient depends on a large number of individual factors, such as, for example, on the efficacy of the particular compound used, on the age, body weight, general state of health, sex, nutrition, on the time and method of administration, on the excretion rate, on the combination with other medicaments and on the severity and duration of the particular disease.

A measure of the uptake of a medicament active compound in an organism is its bioavailability. If the medicament active compound is delivered to the organism intravenously in the form of an injection solution, its absolute bioavailability, i.e. the proportion of the pharmaceutical which reaches the systemic blood, i.e. the major circulation, in unchanged form, is 100%. In the case of oral administration of a therapeutic active compound, the active compound is generally in the form of a solid in the formulation and must therefore first be dissolved in order that it is able to overcome the entry barriers, for example the gastrointestinal tract, the oral mucous membrane, nasal membranes or the skin, in particular the stratum corneum, or can be absorbed by the body. Data on the pharmacokinetics, i.e. on the bioavailability, can be obtained analogously to the method of J. Shaffer et al., J. Pharm. Sciences, 88 (1999), 313-318.

Furthermore, medicaments of this type can be prepared by means of one of the processes generally known in the pharmaceutical art.

Medicaments can be adapted for administration via any desired suitable route, for example by the oral (including buccal or sublingual), rectal, pulmonary, nasal, topical (including buccal, sublingual or transdermal), vaginal or parenteral (including subcutaneous, intramuscular, intravenous, intradermal and in particular intra-articular) routes. Medicaments of this type can be prepared by means of all processes known in the pharmaceutical art by, for example, combining the active compound with the excipient(s) or adjuvant(s).

Parenteral administration is preferably suitable for administration of the medicaments according to the invention. In the case of parenteral administration, intra-articular administration is particularly preferred.

The compounds according to the invention are also suitable for the preparation of medicaments to be administered parenterally having slow, sustained and/or controlled release of active compound. They are thus also suitable for the preparation of delayed-release formulations, which are advantageous for the patient since administration is only necessary at relatively large time intervals.

The medicaments adapted to parenteral administration include aqueous and non-aqueous sterile injection solutions comprising antioxidants, buffers, bacteriostatics and solutes, by means of which the formulation is rendered isotonic with the blood or synovial fluid of the recipient to be treated; as well as aqueous and non-aqueous sterile suspensions, which can comprise suspension media and thickeners. The formulations can be delivered in single-dose or multi-dose containers, for example sealed ampoules and vials, and stored in the freeze-dried (lyophilised) state, so that only the addition of the sterile carrier liquid, for example water for injection purposes, immediately before use is necessary. Injection solutions and suspensions prepared in accordance with the formulation can be prepared from sterile powders, granules and tablets.

The compounds according to the invention can also be administered in the form of liposome delivery systems, such as, for example, small unilamellar vesicles, large unilamellar vesicles and multilamellar vesicles. Liposomes can be formed from various phospholipids, such as, for example, cholesterol, stearylamine or phosphatidylcholines.

The compounds according to the invention can also be coupled to soluble polymers as targeted medicament excipients. Such polymers can encompass polyvinylpyrrolidone, pyran copolymer, polyhydroxypropylmethacrylamidophenol, polyhydroxyethylaspartamidophenol or polyethylene oxide polylysine, substituted by palmitoyl radicals. The compounds according to the invention can furthermore be coupled to a class of biodegradable polymers which are suitable for achieving slow release of a medicament, for example polylactic acid, poly-epsilon-caprolactone, polyhydroxybutyric acid, polyorthoesters, polyacetals, polydihydroxypyrans, poly-cyanoacrylates, polylactic-co-glycolic acid, polymers, such as conjugates between dextran and methacrylates, polyphosphoesters, various polysaccharides and poly-amines and poly-E-caprolactone, albumin, chitosan, collagen or modified gelatine and crosslinked or amphipathic block copolymers of hydrogels.

Suitable for enteral administration (oral or rectal) are, in particular, tablets, dragees, capsules, syrups, juices, drops or suppositories, and suitable for topical use are ointments, creams, pastes, lotions, gels, sprays, foams, aerosols, solutions (for example solutions in alcohols, such as ethanol or isopropanol, acetonitrile, DMF, dimethylacetamide, 1,2-propanediol or mixtures thereof with one another and/or with water) or powders. Also, particularly suitable for topical uses are liposomal preparations.

In the case of formulation to give an ointment, the active compound can be employed either with a paraffinic or a water-miscible cream base. Alternatively, the active compound can be formulated to a cream with an oil-in-water cream base or a water-in-oil base.

Medicaments adapted to transdermal administration can be delivered as independent plasters for extended, close contact with the epidermis of the recipient.

Thus, for example, the active compound can be supplied from the plaster by means of iontophoresis, as described in general terms in Pharmaceutical Research, 3 (6), 318 (1986).

It goes without saying that, besides the constituents particularly mentioned above, the medicaments according to the invention may also comprise other agents usual in the art with respect to the particular type of pharmaceutical formulation.

The invention also relates to a set (kit) consisting of separate packs of
a) an effective amount of a compound of the present invention and/or physiologi-cally acceptable salts, derivatives, solvates, prodrugs and stereoisomers thereof, including mixtures thereof in all ratios, and
b) an effective amount of a further medicament active compound.

The set comprises suitable containers, such as boxes or cartons, individual bottles, bags or ampoules. The set may, for example, comprise separate ampoules each containing an effective amount of a compound of the present inventionand/or pharmaceutically acceptable salts, derivatives, solvates, prodrugs and stereoisom-ers thereof, including mixtures thereof in all ratios, and an effective amount of a further medicament active compound in dissolved or lyophilised form.

Furthermore, the medicaments according to the invention can be used in order to provide additive or synergistic effects in certain known therapies and/or can be used in order to restore the efficacy of certain existing therapies.

Besides the compounds according to the invention, the pharmaceutical preparations according to the invention may also comprise further medicament active compounds, for example for use in the treatment of cancer, other anti-tumor medicaments. For the treatment of the other diseases mentioned, the pharmaceutical preparations according to the invention may also, besides the compounds according to the invention, comprise further medicament active compounds which are known to the person skilled in the art in the treatment thereof. In one principal embodiment, methods are provided for enhancing an immune response in a host in need thereof. The immune response can be enhanced by reducing T cell tolerance, including by increasing IFN-γ release, by decreasing regulatory T cell production or activation, or by increasing antigen-specific memory T cell production in a host. In one embodiment, the method comprises administering a compound of the present invention to a host in combination or alternation with an antibody. In particular subembodiments, the antibody is a therapeutic antibody. In one particular embodiment, a method of enhancing efficacy of passive antibody therapy is provided comprising administering a compound of the present invention in combination or alternation with one or more passive antibodies. This method can enhance the efficacy of antibody therapy for treatment of abnormal cell proliferative disorders such as cancer or can enhance the efficacy of therapy in the treatment or prevention of infectious diseases. The compound of the present invention can be administered in combination or alternation with antibodies such as rituximab, herceptin or erbitux, for example.

In another principal embodiment, a method of treating or preventing abnormal cell proliferation is provided comprising administering a compound of the present invention to a host in need thereof substantially in the absence of another anti-cancer agent.

In another principal embodiment, a method of treating or preventing abnormal cell proliferation in a host in need thereof is provided, comprising administering a first compound of the present invention substantially in combination with a first anti-cancer agent to the host and subsequently administering a second compound of the present invention receptor antagonist. In one subembodiment, the second antagonist is administered substantially in the absence of another anti-cancer agent. In another principal embodiment, a method of treating or preventing abnormal cell proliferation in a host in need thereof is provided, comprising administering a compound of the present invention substantially in combination with a first anti-cancer agent to the host and subsequently administering a second anti-cancer agent in the absence of the antagonist.

Thus, the cancer treatment disclosed here can be carried out as therapy with a compound of the present invention or in combination with an operation, irradiation or chemotherapy. Chemotherapy of this type can include the use of one or more active compounds of the following categories of antitumour active compounds: (i) antiproliferative/antineoplastic/DNA-damaging active compounds and combi-nations thereof, as used in medical oncology, such as alkylating active compounds (for example cis-platin, parboplatin, cyclophosphamide, nitrogen mustard, melphalan, chlorambucil, busulphan and nitrosoureas); antimetabolites (for example antifolates such as fluoropyrimidines such as 5-fluorouracil and tegafur, raltitrexed, methotrexate, cytosine arabinoside, hydroxyurea and gemcitabine); antitumour antibiotics (for example anthracyclines, such as adriamycin, bleomycin, doxorubicin, daunomycin, epirubicin, idarubicin, mitomycin-C, dactinomycin and mithramycin); antimitotic active compounds (for example vinca alkaloids, such as vincristine, vin-blastine, vindesine and vinorelbine, and taxoids, such as taxol and taxotere); topoisomerase inhibitors (for example epipodophyllotoxins, such as etoposide and teniposide, amsacrine, topotecan, irinotecan and camptothecin) and cell-differentiating active compounds (for example all-trans-retinoic acid, 13-cis-retinoic acid and fenretinide);
(ii) cytostatic active compounds, such as anti-oestrogens (for example tamoxifen, toremifene, raloxifene, droloxifene and iodoxyfene), oestrogen receptor regulators (for example fulvestrant), anti-androgens (for example bicalutamide, flutamide, nilutamide and cyproterone acetate), LHRH antagonists or LHRH agonists (for example goserelin, leuprorelin and buserelin), progesterones (for example megestrol acetate), aromatase inhibitors (for example anastrozole, letrozole, vorazole and exemestane) and inhibitors of 5α-reductase, such as finasteride; (iii) active compounds which inhibit cancer invasion including for example metallo-proteinase inhibitors, like marimastat, and inhibitors of urokinase plasminogen activator receptor function;
(iv) inhibitors of growth factor function, for example growth factor antibodies, growth factor receptor antibodies, for example the anti-erbb2 antibody trastuzumab [Herceptin™] and the anti-erbbl antibody cetuximab [C225]), farnesyl transferase inhibitors, tyrosine kinase inhibitors and serine/threonine kinase inhibitors, for example inhibitors of the epidermal growth factor family (for example EGFR family tyrosine kinase inhibitors, such as N-(3-chloro-4-fluorophenyl)-7-methoxy-6-(3-morpholinopropoxy) quinazolin-4-amine (gefitinib, AZD1839), N-(3-ethynylphenyl)-6,7-bis(2-methoxyethoxy)quinazolin-4-amine (erlotinib, OSI-774) and 6-acrylamido-N-(3-chloro-4-fluorophenyl)-7-(3-morpholinopropoxy)quinazolin-4-amine (Cl 1033), for example inhibitors of the platelet-derived growth factor family and, for example, inhibitors of the hepatocyte growth factor family;
(v) anti-angiogenic active compounds, such as bevacizumab, angiostatin, endostatin, linomide, batimastat, captopril, cartilage derived inhibitor, genistein, interleukin 12, lavendustin, medroxypregesterone acetate, recombinant human platelet factor 4, tecogalan, thrombospondin, TNP-470, anti-VEGF monoclonal antibody, soluble VEGF-receptor chimaeric protein, anti-VEGF receptor antibodies, anti-PDGF receptors, inhibitors of integrins, tyrosine kinase inhibitors, serine/threonine kinase inhibitors, antisense oligonucleotides, antisense oligodexoynucleotides, siRNAs, anti-VEGF aptamers, pigment epithelium derived factor and compounds which have been published in the international patent applications WO 97/22596, WO 97/30035, WO 97/32856 and WO 98/13354);
(vi) vessel-destroying agents, such as combretastatin A4 and compounds which have been published in the international patent applications WO 99/02166, WO 00/40529, WO 00/41669, WO 01/92224, WO 02/04434 and WO 02/08213;
(vii) antisense therapies, for example those directed to the targets mentioned above, such as ISIS 2503, an anti-Ras antisense;
(viii) gene therapy approaches, including, for example, approaches for replacement of abnormal, modified genes, such as abnormal p53 or abnormal BRCA1 or BRCA2, GDEPT approaches (gene-directed enzyme pro-drug therapy), such as those which use cytosine deaminase, thymidine kinase or a bacterial nitroreductase enzyme, and approaches which increase the tolerance of a patient to chemotherapy or radiotherapy, such as multi-drug resistance therapy; and
(ix) immunotherapy approaches, including, for example, ex-vivo and in-vivo approaches for increasing the immunogenicity of tumour cells of a patient, such as transfection with cytokines, such as interleukin 2, interleukin 4 or granulocyte macrophage colony stimulating factor, approaches for decreasing T-cell anergy, approaches using transfected immune cells, such as cytokine-transfected dendritic cells, approaches for use of cytokine-transfected tumour cells and approaches for use of anti-idiotypic antibodies
(x) chemotherapeutic agents including foor example abarelix, aldesleukin, alemtuzumab, alitretinoin, allopurinol, altretamine, amifostine, anastrozole, arsenic trioxide, asparaginase, BCG live, bevaceizumab, bexarotene, bleomycin, bortezomib, busulfan, calusterone, camptothecin, capecitabine, carboplatin, carmustine, celecoxib, cetuximab, chlorambucil, cinacalcet, cisplatin, cladribine, cyclophosphamide, cytarabine, dacarbazine, dactinomycin, darbepoetin alfa, daunorubicin, denileukin diftitox, dexrazoxane, docetaxel, doxorubicin, dromostanolone, epirubicin, epoetin alfa, estramustine, etoposide, exemestane, filgrastim, floxuridine, fludarabine, fluorouracil, fulvestrant and gemcitabine.

The medicaments from table 1 can preferably, but not exclusively, be combined with the compounds of the present invention.

TABLE 1

| | | |
|---|---|---|
| Alkylating active | Cyclophosphamide | Lomustine |
| compounds | Busulfan | Procarbazine |
| | Ifosfamide | Altretamine |
| | Melphalan | Estramustine phosphate |
| | Hexamethylmelamine | Mechloroethamine |
| | Thiotepa | Streptozocin |
| | chloroambucil | Temozolomide |
| | Dacarbazine | Semustine |
| | Carmustine | |
| Platinum active | Cisplatin | Carboplatin |
| compounds | Oxaliplatin | ZD-0473 (AnorMED) |
| | Spiroplatin | Lobaplatin (Aetema) |
| | Carboxyphthalatoplatinum | Satraplatin (Johnson |
| | Tetraplatin | Matthey) |
| | Ormiplatin | BBR-3464 |
| | Iproplatin | (Hoffrnann-La Roche) |
| | | SM-11355 (Sumitomo) |
| | | AP-5280 (Access) |
| Antimetabolites | Azacytidine | Tomudex |
| | Gemcitabine | Trimetrexate |
| | Capecitabine | Deoxycoformycin |
| | 5-Fluorouracil | Fludarabine |
| | Floxuridine | Pentostatin |
| | 2-Chlorodesoxyadenosine | Raltitrexed |
| | 6-Mercaptopurine | Hydroxyurea |
| | 6-Thioguanine | Decitabine (SuperGen) |
| | Cytarabine | Clofarabine (Bioenvision) |
| | 2-Fluorodesoxycytidine | Irofulven (MGI Pharrna) |
| | Methotrexate | DMDC (Hoffmann-La Roche) |
| | Idatrexate | Ethynylcytidine (Taiho ) |
| Topoisomerase | Amsacrine | Rubitecan (SuperGen) |
| inhibitors | Epirubicin | Exatecan mesylate (Daiichi) |
| | Etoposide | Quinamed (ChemGenex) |
| | Teniposide or mitoxantrone | Gimatecan (Sigma- Tau) |
| | Irinotecan (CPT-11) | Diflomotecan (Beaufour- |
| | 7-ethyl-10- | Ipsen) |
| | hydroxycamptothecin | TAS-103 (Taiho) |
| | Topotecan | Elsamitrucin (Spectrum) |
| | Dexrazoxanet (TopoTarget) | J-107088 (Merck & Co) |
| | Pixantrone (Novuspharrna) | BNP-1350 (BioNumerik) |
| | Rebeccamycin analogue | CKD-602 (Chong Kun Dang) |
| | (Exelixis) | KW-2170 (Kyowa Hakko) |
| | BBR-3576 (Novuspharrna) | |
| Antitumour | Dactinomycin (Actinomycin | Amonafide |
| antibiotics | D) | Azonafide |
| | Doxorubicin (Adriamycin) | Anthrapyrazole |
| | Deoxyrubicin | Oxantrazole |
| | Valrubicin | Losoxantrone |
| | Daunorubicin (Daunomycin) | Bleomycin sulfate (Blenoxan) |
| | Epirubicin | Bleomycinic acid |
| | Therarubicin | Bleomycin A |
| | Idarubicin | Bleomycin B |
| | Rubidazon | Mitomycin C |
| | Plicamycinp | MEN-10755 (Menarini) |
| | Porfiromycin | GPX-100 (Gem |
| | Cyanomorpholinodoxorubicin | Pharmaceuticals) |
| | Mitoxantron (Novantron) | |
| Antimitotic active | Paclitaxel | SB 408075 |
| compounds | Docetaxel | (GlaxoSmithKline) |
| | Colchicine | E7010 (Abbott) |
| | Vinblastine | PG-TXL (Cell Therapeutics) |
| | Vincristine | IDN 5109 (Bayer) |
| | Vinorelbine | A 105972 (Abbott) |
| | Vindesine | A 204197 (Abbott) |
| | Dolastatin 10 (NCI) | LU 223651 (BASF) |
| | Rhizoxin (Fujisawa) | D 24851 (ASTA Medica) |
| | Mivobulin (Warner-Lambert) | ER-86526 (Eisai) |
| | Cemadotin (BASF) | Combretastatin A4 (BMS) |
| | RPR 109881A (Aventis) | Isohomohalichondrin-B |
| | TXD 258 (Aventis) | (PharmaMar) |
| | Epothilone B (Novartis) | ZD 6126 (AstraZeneca) |
| | T 900607 (Tularik) | PEG-Paclitaxel (Enzon) |
| | T 138067 (Tularik) | AZ10992 (Asahi) |
| | Cryptophycin 52 (Eli Lilly) | !DN-5109 (Indena) |
| | Vinflunine (Fabre) | AVLB (Prescient |
| | Auristatin PE (Teikoku | NeuroPharma) |
| | Hormone) | Azaepothilon B (BMS) |
| | BMS 247550 (BMS) | BNP- 7787 (BioNumerik) |
| | BMS 184476 (BMS) | CA-4-prodrug (OXIGENE) |
| | BMS 188797 (BMS) | Dolastatin-10 (NrH) |
| | Taxoprexin (Protarga) | CA-4 (OXIGENE) |
| Aromatase | Aminoglutethimide | Exemestan |
| inhibitors | Letrozole | Atamestan (BioMedicines) |
| | Anastrazole | YM-511 (Yamanouchi) |
| | Formestan | |
| Thymidylate | Pemetrexed (Eli Lilly) | Nolatrexed (Eximias) |
| Synthase | ZD-9331 (BTG) | CoFactor ™ (BioKeys) |
| inhibitors | | |
| DNA antagonists | Trabectedin (PharmaMar) | Mafosfamide (Baxter |
| | Glufosfamide (Baxter | International) |
| | International) | Apaziquone (Spectrum |
| | Albumin + 32P | Pharmaceuticals) |
| | (isotope solutions) | O6-benzylguanine (Paligent) |
| | Thymectacin (NewBiotics) | |
| | Edotreotid (Novartis) | |
| Farnesyl transferase | Arglabin (NuOncology Labs) | Tipifarnib (Johnson & |
| inhibitors | Lonafarnib (Schering-Plough) | Johnson) |
| | BAY-43-9006 (Bayer) | Perillyl alcohol (DOR |
| | | BioPharma) |
| Pump inhibitors | CBT-1 (CBA Pharma) | Zosuquidar trihydrochloride |
| | Tariquidar (Xenova) | (Eli Lilly) |
| | MS-209 (Schering AG) | Biricodar dicitrate (Vertex) |
| Histone acetyl trans- | Tacedinaline (Pfizer) | Pivaloyloxymethyl butyrate |
| ferase inhibitors | SAHA (Aton Pharma) | (Titan) |
| | MS-275 (Schering AG) | Depsipeptide (Fujisawa) |
| Metalloproteinase | Neovastat (Aeterna | CMT -3 (CollaGenex) |
| inhibitors | Laboratories) | BMS-275291 (Celltech) |
| Ribonucleoside | Marimastat (British Biotech) | Tezacitabine (Aventis) |
| reductase | Gallium maltolate (Titan) | Didox (Molecules for Health) |
| inhibitors | Triapin (Vion) | |
| TNF-alpha | Virulizin (Lorus Therapeutics) | Revimid (Celgene) |
| agonists/ | CDC-394 (Celgene) | |
| antagonists | | |
| Endothelin-A re- | Atrasentan (Abbot) | YM-598 (Yamanouchi) |
| ceptor antagonists | ZD-4054 (AstraZeneca) | |
| Retinoic acid | Fenretinide (Johnson & | Alitretinoin (Ligand) |
| receptor agonists | Johnson) | |
| | LGD-1550 (ligand) | |
| Immunomodulators | Interferon | Dexosome therapy (Anosys) |
| | Oncophage (Antigenics) | Pentrix (Australian Cancer |
| | GMK (Progenics) | Technology) |
| | Adenocarcinoma vaccine | JSF-154 (Tragen) |
| | (Biomira) | Cancer vaccine (Intercell) |
| | CTP-37 (AVI BioPharma) | Norelin (Biostar) |
| | JRX-2 (Immuno-Rx) | BLP-25 (Biomira) |
| | PEP-005 (Peplin Biotech) | MGV (Progenics) |
| | Synchrovax vaccines (CTL | !3-Alethin (Dovetail) |
| | Immuno) | CLL-Thera (Vasogen) |
| | Melanoma vaccines (CTL | |
| | Immuno) | |
| | p21-RAS vaccine (GemVax) | |
| Hormonal and | Oestrogens | Prednisone |
| antihormonal active | Conjugated oestrogens | Methylprednisolone |
| compounds | Ethynyloestradiol | Prednisolone |
| | Chlorotrianisene | Aminoglutethimide |
| | Idenestrol | Leuprolide |
| | Hydroxyprogesterone | Goserelin |
| | caproate | Leuporelin |
| | Medroxyprogesterone | Bicalutamide |
| | Testosterone | Flutamide |
| | Testosterone propionate | Octreotide |
| | Fluoxymesterone | Nilutamide |
| | Methyltestosterone | Mitotan |
| | Diethylstilbestrol | P-04 (Novogen) |
| | Megestrol | 2-Methoxyoestradiol (En _- |
| | Tamoxifen | treMed) |
| | Toremofin | Arzoxifen (Eli Lilly) |
| | Dexamethasone | |
| Photodynamic | Talaporfin (Light Sciences) | Pd bacteriopheophorbide |
| active compounds | Theralux (Theratechnologies) | (Yeda) |
| | Motexafin-Gadolinium | Lutetium texaphyrin |
| | (Pharmacyclics) | (Pharmacyclics) |
| | | Hypericin |
| Tyrosine kinase | Imatinib (Novartis) | CEP- 701 (Cephalon) |
| inhibitors | Leflunomide(Sugen/Pharmacia] | Kahalide F (PharmaMar) |
| | ZDI839 (AstraZeneca) | CEP-751 (Cephalon) |
| | Erlotinib (Oncogene Science) | MLN518 (Millenium) |
| | Canertjnib (Pfizer) | PKC412 (Novartis) |
| | Squalamine (Genaera) | Phenoxodiol O |
| | SU5416 (Pharmacia) | Trastuzumab (Genentech) |
| | SU6668 (Pharmacia) | C225 (ImClone) |
| | ZD4190 (AstraZeneca) | rhu-Mab (Genentech) |
| | ZD6474 (AstraZeneca) | MDX-H210 (Medarex) |
| | Vatalanib (Novartis) | 2C4 (Genentech) |
| | PKI166 (Novartis) | MDX-447 (Medarex) |
| | GW2016 (GlaxoSmithKline) | ABX-EGF (Abgenix) |
| | EKB-509 (Wyeth) | IMC-1C11 (ImClone) |
| | EKB-569 (Wyeth) | |
| Various other active | SR-27897 (CCK-A inhibitor, | BCX-1777 (PNP inhibitor, |
| compounds | Sanofi-Synthelabo) | BioCryst) |
| | Tocladesine (cyclic AMP | Ranpirnase (ribonuclease |
| | agonist, Ribapharm) | stimulant, Alfacell) |
| | Alvocidib (CDK inhibitor, | Galarubicin (RNA synthesis |
| | Aventis) | inhibitor, Dong-A) |
| | CV-247 (COX-2 inhibitor, Ivy | Tirapazamine (reducing |
| | Medical) | agent, SRI International) |
| | P54 (COX-2 inhibitor, | N-Acetylcysteine |
| | Phytopharm) | (reducing agent, |
| | CapCell ™ (CYP450 | Zambon) |
| | stimulant, Bavarian Nordic) | R-Flurbiprofen (NF-kappaB |
| | GCS-IOO (gal3 antagonist, | inhibitor, Encore) |
| | GlycoGenesys) | 3CPA (NF-kappaB inhibitor, |
| | G17DT immunogen (gastrin | Active Biotech) |
| | inhibitor, Aphton) | Seocalcitol (vitamin D |
| | Efaproxiral (oxygenator, | receptor agonist, Leo) |
| | Allos Therapeutics) | 131-I-TM-601 (DNA |
| | PI-88 (heparanase inhibitor, | antagonist, TransMolecular) |
| | Progen) | Eflornithin (ODC inhibitor, |
| | Tesmilifen (histamine | ILEX Oncology) |
| | antagonist, YM BioSciences) | Minodronic acid (osteoclast |
| | Histamine (histamine H2 | inhibitor, |
| | receptor agonist, Maxim) | Yamanouchi) |
| | Tiazofurin (IMPDH inhibitor, | Indisulam (p53 stimulant, |
| | Ribapharm) | Eisai) |
| | Cilengitide (integrin antagonist, | Aplidin (PPT inhibitor, |
| | Merck KGaA) | PharmaMar) |
| | SR-31747 (IL-1 antagonist, | Rituximab (CD20 antibody, |
| | Sanofi-Synthelabo) | Genentech) |
| | CCI-779 (mTOR kinase | Gemtuzumab (CD33 |
| | inhibitor, Wyeth) | antibody, Wyeth Ayerst) |
| | Exisulind (PDE-V inhibitor, | PG2 (haematopoiesis |
| | Cell Pathways) | promoter, Pharmagenesis) |
| | CP-461 (PDE-V inhibitor, Cell | Immunol ™ (triclosan |
| | Pathways) | mouthwash, Endo) |
| | AG-2037 (GART inhibitor, | Triacetyluridine (uridine |
| | Pfizer) | prodrug, Wellstat) |
| | WX-UK1 (plasminogen | SN-4071 (sarcoma agent, |
| | activator inhibitor, Wilex) | Signature BioScience) |
| | PBI-1402 (PMN stimulant, | TransMID-107 ™ M |
| | ProMetic LifeSciences) | (immunotoxin, KS Biomedix) |
| | Bortezomib (proteasome | PCK-3145 (apoptosis pro- |
| | inhibitor, Millennium) | moter, Procyon) |
| | SRL-172 (T-cell stimulant, | Doranidazole (apoptosis pro- |
| | SR Pharma) | moter, Pola) |
| | TLK-286 (glutathione-S | CHS-828 (cytotoxic agent, |
| | transferase inhibitor, Telik) | Leo) |
| | PT-100 (growth factor | trans-Retinoic acid |
| | agonist, Point Therapeutics) | (differentiator, NIH) |
| | Midostaurin (PKC inhibitor, | MX6 (apoptosis promoter, |
| | Novartis) | MAXIA) |
| | Bryostatin-1 (PKC stimulant, | Apomine (apoptosis |
| | GPC Biotech) | promoter, ILEX Oncology) |
| | CDA-II (apoptosis promoter, | Urocidin (apoptosis promoter, |
| | Everlife) | Bioniche) |
| | SDX-101 (apoptosis promoter, | Ro-31-7453 (apoptosis pro- |
| | Salmedix) | moter, La Roche) |
| | Ceflatonin (apoptosis pro- | Brostallicin (apoptosis |
| | moter, ChemGenex) | promoter, Pharmacia) |

Even without further embodiments, it is assumed that a person skilled in the art will be able to use the above description in the broadest scope. The preferred embodiments should therefore merely be regarded as descriptive disclosure which is absolutely not limiting in any way.

The following examples are thus intended to explain the invention without limiting it. Unless indicated otherwise, percent data denote percent by weight. All temperatures are indicated in degrees Celsius. “Conventional work-up”: water is added if necessary, the pH is adjusted, if necessary, to values between 2 and 10, depending on the constitution of the end product, the mixture is extracted with ethyl acetate or dichloromethane, the phases are separated, the organic phase is dried over sodium sulfate or magnesium sulfate, filtered and evaporated, and the product is purified by chromatography on silica gel and/or by crystallisation.

Rf values on silica gel; mass spectrometry: El (electron impact ionisation): M+, FAB (fast atom bombardment): (M+H)+, THE (tetrahydrofuran), NMP (N-methlpyrrolidone), DMSO (dimethyl sulfoxide), EtOAc (ethyl acetate), MeOH (methanol), EtOH (ethanol), TLC (thin-layer chromatography)

### List of Abbreviations

AUC Area under the plasma drug concentration-time curve
Cₘₐₓ Maximum plasma concentration
CL Clearance
CV Coefficient of variation
CYP Cytochrome P450
DMSO Dimethyl sulfoxide
F Bioavailability
fₐ Fraction absorbed
iv Intravenous
LC-MS/MS Liquid chromatography tandem mass spectrometry
LLOQ Lower limit of quantification
NC Not calculated
ND Not determined
PEG Polyethylene glycol
Pgp Permeability glycoprotein
PK Pharmacokinetic(s)
po Per os (oral)
rt Room temperature
t_{1/2} Half-life
tₘₐₓ Time at which maximum plasma concentration of drug is reached
UPLC Ultra performance liquid chromatography
Vₛₛ Volume of distribution (at steady state)
v/v Volume to volume

### Preparation of the Compounds of the Present Invention and Analytical Methods

The invention especially relates to the compounds of the following examples and physiologically acceptable salts, derivatives, solvates, prodrugs and stereoisomers thereof, including mixtures thereof in all ratios.

All solvents used were commercially available and used without further purification. Reactions were typically run using anhydrous solvents under an inert atmosphere of nitrogen or argon. Flash column chromatography was carried out using Silica gel 60 (0.035-0.070 mm particle size). Flash column chromatography was also carried out using a Biotage purification system using SNAP KP-Sil cartridges or on reverse-phase mode using SNAP Ultra C18 cartridges. Microwave-assisted reactions were carried out using a Biotage Initiator microwave system.

¹H NMR spectra were recorded on Bruker DPX-300, DRX-400, Avance 11-400, Avance III HD-400, Avance II+-500, Avance III-500, Avance Neo 600 or on a Avance III-700 spectrometer, using residual signal of deuterated solvent as internal reference. Chemical shifts (δ) are reported in ppm relative to tetramethylsilane (TMS), referenced to the internal deuterated solvent. ¹H NMR data are reported as follows: chemical shift (multiplicity, coupling constants, and number of hydrogens). Multiplicity is abbreviated as follows: s (singlet), d (doublet), t (triplet), q (quartet), sext (sextet), hept (heptet), m (multiplet), br (broad).

HPLC/MS spectra of the products were recorded on an Agilent 1100 HPLC system (1100 high pressure gradient pump, 1100 diode array detector, wavelength: 220 nm) interfaced to an Agilent 1100 mass spectrometer detector (positive mode). LC-MS analyses were performed on a SHIMADZU LC-MS machine consisting of an UFLC 20-AD system and LCMS 2020 MS detector.

Details of the applied conditions for HPLC/MS spectra recorded on a Shimadzu LCMS-2020 system or on an Agilent 1100 HPLC system:

(A): column: HALO C18, 3.0*30 mm, 2.0 μm; mobile phase A: water with 0.05% TFA, mobile phase B: ACN with 0.05% TFA; gradient: 5% B to 100% B till min 0.70, hold till min 1.10, 100% B to 5% B till min 1.12, stop after 1.20; flow: 1.5 mL/min.

(B): column: Titank C18 2.1*30 mm, 1.8 μm; mobile Phase A: water with 0.04% NH₄OH, mobile Phase B: ACN; gradient: 10% B to 95% B in 1.4 min, hold till min 1.90, 95% B to 10% B till min 1.92, stop after 2.00; flow: 0.8 mL/min.

(C): column: HALO C18, 3.0*30 mm, 2.0 μm; mobile phase A: water with 0.05% TFA, mobile phase B: ACN with 0.05% TFA; gradient: 5% B to 100% B till min 1.20, hold till min 1.80, 100% B to 5% B till min 1.82, stop after 2.00; flow: 1.5 mL/min.

(D): column: HALO C18, 3.0*30 mm, 2.0 μm; mobile phase A: water with 0.05% TFA, mobile phase B: ACN with 0.05% TFA; gradient: 5% B to 50% B till min 1.80, 50% B to 95% B till min 2.50, hold till min 2.80, 95% B to 5% B till min 2.81, stop after 3.00; flow: 1.5 mL/min.

(E): column: HALO C18, 3.0*30 mm, 2.0 μm; mobile phase A: water with 0.1% FA, mobile phase B: ACN with 0.1% FA; gradient: 10% B to 95% B till min 1.20, hold till min 1.80, 95% B to 10% B till min 1.82, stop after 2.00; flow: 1.5 mL/min.

(F): column: Kinetex EVO C18, 4.6×50 mm, 5.0 μm; mobile phase A: water with 0.05% FA, mobile phase B: ACN with 0.04% FA and 1% water; gradient: 1% B to 99% B till min 0.8, 99% B to 1% B till min 1.1, stop after 1.50; flow: 3.3 mL/min.

(G): column: Kinetex EVO C18, 3.0×50 mm, 2.6 μm; mobile phase A: water with 5 mM NH₄HCO₃, mobile phase B: ACN; gradient: 10% B to 95% B in 2.0 min, hold till min 2.7, 95% B to 10% B till min 2.75, stop after 3.0 flow: 1.2 mL/min; wavelength: 254 nm.

(H): column: Chromolith HR C18, 4.6×50 mm, 5.0 μm; mobile phase A: water with 0.1% TFA, mobile phase B: ACN with 0.1% TFA; gradient: 1% B to 99% B till min 2.0, hold till min 2.5, 99% B to 1% B till min 2.51, stop after 2.95; flow: 3.3 mL/min.

(I): column: Shim-pack velox, 3.0*30 mm, 2.7 μm; mobile phase A: water with 0.1% FA, mobile phase B: ACN with 0.1% FA; gradient: 5% B to 100% B till min 1.50, hold till min 1.80, 100% B to 5% B till min 1.81, stop after 2.00; flow: 1.5 mL/min.

(J): column: HALO C18, 3.0*30 mm, 2.0 μm; mobile phase A: water with 0.05% TFA, mobile phase B: ACN with 0.05% TFA; gradient: 5% B to 95% B till min 2.50, hold till min 2.80, 95% B to 5% B till min 2.81, stop after 3.00; flow: 1.5 mL/min.

(K): column: HALO C18, 3.0*30 mm, 2.0 μm; mobile phase A: water with 0.1% FA, mobile phase B: ACN with 0.1% FA; gradient: 5% B to 95% B till min 2.50, hold till min 2.80, 95% B to 5% B till min 2.81, stop after 3.00; flow: 1.5 mL/min.

(L): column: HALO C18, 3.0*30 mm, 2.0 μm; mobile phase A: water with 0.1% FA, mobile phase B: ACN with 0.1% FA; gradient: 5% B to 100% B till min 1.50, hold till min 1.80, 100% B to 5% B till min 1.81, stop after 2.00; flow: 1.5 mL/min.

(M): column: Chromolith HR C18 RP-18e, 4.6×50 mm; mobile phase A: water with 0.05% FA, mobile phase B: ACN with 0.04% FA; gradient: 0% B to 100% B till min 2.0, hold till min 2.5, 100% B to 0% B till min 2.51, stop after 2.95; flow: 3.3 mL/min.

(N): column: Titank C18 2.1*30 mm, 1.8 μm; mobile Phase A: water with 0.04% NH₄OH, mobile Phase B: ACN; gradient: 10% B to 95% B in 2.0 min, hold till min 2.80, 95% B to 10% B till min 2.81, stop after 3.00; flow: 0.8 mL/min.

(O): column: HALO C18, 3.0*30 mm, 2.0 μm; mobile phase A: water with 0.1% FA, mobile phase B: ACN with 0.1% FA; gradient: 5% B to 95% B till min 2.10, hold till min 2.75, 95% B to 5% B till min 2.81, stop after 3.00; flow: 1.5 mL/min.

Details of the applied conditions for HPLC/MS spectra on an Agilent 1200 series HPLC and diode array detector coupled to a 6210 time of flight mass spectrometer with dual multimode APCI/ESI source.

(P): Analytical separation was carried out at 40° C. on a Merck Chromolith Flash column (RP-18e, 25×2 mm) using a flow rate of 1.5 mL/min in a 2 min gradient elution with detection at 254 nm. The mobile phase was a mixture of methanol (solvent A) and water (solvent B), both containing formic acid at 0.1%. Gradient elution was as follows: 5:95 (A/B) to 100:0 (A/B) over 1.25 min, 100:0 (A/B) for 0.5 min, and then reversion back to 5:95 (A/B) over 0.05 min, finally 5:95 (A/B) for 0.2 min.

(Q): Analytical separation was carried out at 30° C. on a Merck Chromolith Flash column (RP-18e, 25×2 mm) using a flow rate of 0.75 mL/min in a 4 min gradient elution with detection at 254 nm. The mobile phase was a mixture of methanol (solvent A) and water (solvent B), both containing formic acid at 0.1%. Gradient elution was as follows: 5:95 (A/B) to 100:0 (A/B) over 2.5 min, 100:0 (A/B) for 1 min, and then reversion back to 5:95 (A/B) over 0.1 min, finally 5:95 (A/B) for 0.4 min.

Details of the applied conditions for HPLC/MS spectra on a Waters Acquity UPLC and diode array detector coupled to a Waters G2 QToF mass spectrometer fitted with a multimode ESI/APCI source.

(R): Analytical separation was carried out at 30° C. on a Phenomenex Kinetex C18 column (30×2.1 mm, 2.6u, 100A) using a flow rate of 0.5 mL/min in a 2 min gradient elution with detection at 254 nm. The mobile phase was a mixture of methanol (solvent A) and water (solvent B), both containing formic acid at 0.1%. Gradient elution was as follows: 10:90 (A/B) to 90:10 (A/B) over 1.25 min, 90:10 (A/B) for 0.5 min, and then reversion back to 10:90 (A/B) over 0.15 min, finally 10:90 (A/B) for 0.1 min.

(S): Analytical separation was carried out at 30° C. on a Phenomenex Kinetex C18 column (30×2.1 mm, 2.6u, 100A) using a flow rate of 0.3 mL/min in a 4 min gradient elution with detection at 254 nm. The mobile phase was a mixture of methanol (solvent A) and water (solvent B), both containing formic acid at 0.1%. Gradient elution was as follows: 10:90 (A/B) to 90:10 (A/B) over 3 min, 90:10 (A/B) for 0.5 min, and then reversion back to 10:90 (A/B) over 0.3 min, finally 10:90 (A/B) for 0.2 min.

(T): Analytical separation was carried out at 30° C. on an Agilent Poroshell C18 column (30×2.1 mm, 2.6u, 100A) using a flow rate of 0.5 mL/min in a 2 min gradient elution with detection at 254 nm. The mobile phase was a mixture of methanol (solvent A) and water (solvent B), both containing formic acid at 0.1%. Gradient elution was as follows: 10:90 (A/B) to 90:10 (A/B) over 1.25 min, 90:10 (A/B) for 0.5 min, and then reversion back to 10:90 (A/B) over 0.15 min, finally 10:90 (A/B) for 0.1 min.

(U): Analytical separation was carried out at 30° C. on an Agilent Poroshell C18 column (30×2.1 mm, 2.6u, 100A) using a flow rate of 0.3 mL/min in a 4 min gradient elution with detection at 254 nm. The mobile phase was a mixture of methanol (solvent A) and water (solvent B), both containing formic acid at 0.1%. Gradient elution was as follows: 10:90 (A/B) to 90:10 (A/B) over 3 min, 90:10 (A/B) for 0.5 min, and then reversion back to 10:90 (A/B) over 0.3 min, finally 10:90 (A/B) for 0.2 min.

Details of the applied conditions for HPLC/MS spectra on an Agilent 1260 Infinity II series UPLC and diode array detector coupled to a 6530 Quadrupole time of flight mass spectrometer with Agilent Jet Stream ESI source.

(V): Analytical separation was carried out at 40° C. on an Agilent Poroshell C18 column (30×2.1 mm, 2.6u, 100A) using a flow rate of 0.6 mL/min in a 2 min gradient elution with detection at 254, 280 and 214 nm. The mobile phase was a mixture of methanol (solvent A) and water (solvent B), both containing formic acid at 0.1%. Gradient elution was as follows: 10:90 (A/B) to 90:10 (A/B) over 1.25 min, 90:10 (A/B) for 0.5 min, and then reversion back to 10:90 (A/B) over 0.15 min, finally 10:90 (A/B) for 0.1 min.

(X): Analytical separation was carried out at 40° C. on an Agilent Poroshell C18 column (30×2.1 mm, 2.6u, 100A) using a flow rate of 0.4 mL/min in a 4 min gradient elution with detection at 254, 280 and 214 nm. The mobile phase was a mixture of methanol (solvent A) and water (solvent B), both containing formic acid at 0.1%. Gradient elution was as follows: 10:90 (A/B) to 90:10 (A/B) over 2.5 min, 90:10 (A/B) for 1 min, and then reversion back to 10:90 (A/B) over 0.3 min, finally 10:90 (A/B) for 0.2 min.

(Y): Analytical separation was carried out at 40° C. on a Phenomenex Kinetex C18 column (30×2.1 mm, 2.6u, 100A) using a flow rate of 0.6 mL/min in a 2 min gradient elution with detection at 254, 280 and 214 nm. The mobile phase was a mixture of methanol (solvent A) and water (solvent B), both containing formic acid at 0.1%. Gradient elution was as follows: 10:90 (A/B) to 90:10 (A/B) over 1.25 min, 90:10 (A/B) for 0.5 min, and then reversion back to 10:90 (A/B) over 0.15 min, finally 10:90 (A/B) for 0.1 min.

(Z): Analytical separation was carried out at 40° C. on a Phenomenex Kinetex C18 column (30×2.1 mm, 2.6u, 100A) using a flow rate of 0.4 mL/min in a 4 min gradient elution with detection at 254, 280 and 214 nm. The mobile phase was a mixture of methanol (solvent A) and water (solvent B), both containing formic acid at 0.1%. Gradient elution was as follows: 10:90 (A/B) to 90:10 (A/B) over 2.5 min, 90:10 (A/B) for 1 min, and then reversion back to 10:90 (A/B) over 0.3 min, finally 10:90 (A/B) for 0.2 min.

### Example 1: Ethyl 4-methyl-2-(3-(3-methylbenzamido)propanamido)thiazole-5-carboxylate

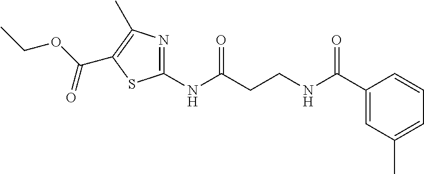 [SMILES: CCOC(=O)c1sc(NC(=O)CCNC(=O)c2cccc(C)c2)nc1C]

Example 1.1.: To a stirred solution of 3-((tert-butoxycarbonyl)amino)propanoic acid (2 g, 10.57 mmol), and HOBt (3.24 g, 21.14 mmol) in dry DMF, under nitrogen atmosphere at rt, were added EDC (4.05 g, 21.14 mmol) and ethyl 2-amino-4-methylthiazole-5-carboxylate (2.165 g, 11.63 mmol) sequentially. The mixture was stirred at 50° C. overnight. The mixture was concentrated in vacuo. The crude product was dissolved in EtOAc and washed with water, 1 N HCl, aqueous saturated bicarbonate, and brine. The organic layer was dried over sodium sulfate and concentrated in vacuo, to give ethyl 2-[3-(tert-butoxycarbonylamino)-propanoylamino]-4-methyl-thiazole-5-carboxylate (3.428 g, 91%, 9.59 mmol) as a yellow solid. HPLC/MS m/z: 358.1 [M+H]⁺, Rt (R): 1.32 min.

Example 1.2.: 4M HCl in dioxane (26.86 mL, 107.43 mmol) was added dropwise to a solution of ethyl 2-[3-(tert-butoxycarbonylamino)propanoylamino]-4-methyl-thiazole-5-carboxylate (1.28 g, 3.5811 mmol) in EtOH (35.81 mL). The reaction mixture was stirred at rt for 2 h before concentrating in vacuo. Purification by ion exchange SCX-II column chromatography, washing with MeOH before eluting with 2M NH₃ gave ethyl 2-(3-aminopropanoylamino)-4-methyl-thiazole-5-carboxylate (867.4 mg, 94%, 3.371 mmol) as a very light yellow solid. HPLC/MS m/z: 258.1 [M+H]⁺, Rt (S): 1.52 min.

Example 1.3.: To 3-methylbenzoic acid (38.1 mg, 0.280 mmol), ethyl 2-(3-aminopropanoylamino)-4-methyl-thiazole-5-carboxylate (80 mg, 0.311 mmol) and DIPEA (163 μl, 0.933 mmol) in DMF (3109 μl) was added HATU (110 mg, 0.466 mmol) and the reaction mixture stirred overnight at rt. The mixture was concentrated in vacuo. The crude was dissolved in EtOAc and washed with 1 N HCl, aqueous saturated bicarbonate, and brine. The organic layer was dried over sodium sulfate, concentrated in vacuo and purified by reverse phase column chromatography (eluent: 0-80% methanol/water+0.1% formic acid in each) to afford 83 mg (71%, 0.221 mmol) of ethyl 4-methyl-2-(3-(3-methylbenzamido)-propanamido)thiazole-5-carboxylate as a colorless solid. HPLC/MS m/z: 376.2 [M+H]⁺, Rt (P): 1.50 min. ¹H NMR (500 MHz, DMSO-d₆) δ 12.51 (s, 1H), 8.55 (t, J=5.6 Hz, 1H), 7.64 (h, J=0.9 Hz, 1H), 7.60 (ddd, J=5.8, 3.4, 1.8 Hz, 1H), 7.34-7.31 (m, 2H), 4.23 (q, J=7.1 Hz, 2H), 3.60-3.51 (m, 2H), 2.75 (t, J=6.9 Hz, 2H), 2.53 (s, 3H), 2.34 (s, 3H), 1.27 (t, J=7.1 Hz, 3H).

The following examples were prepared analogously:

### Example 2: Ethyl 4-methyl-2-(3-(3-(pyrrolidin-1-yl)benzamido)propanamido)thiazole-5-carboxylate

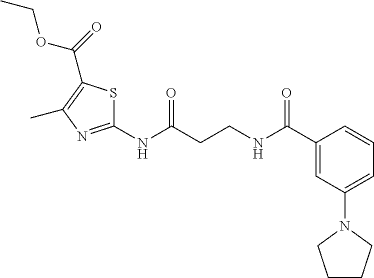 [SMILES: CCOC(=O)c1sc(NC(=O)CCNC(=O)c2cccc(N3CCCC3)c2)nc1C]

14 mg (26%, 0.0325 mmol) yellow solid. HPLC/MS m/z: 431.2 [M+H]⁺, Rt (S): 3.05 min. ¹H NMR (500 MHz, DMSO-d₆) δ 12.46 (s, 1H), 8.45 (t, J=5.6 Hz, 1H), 7.20 (t, J=7.9 Hz, 1H), 7.01 (d, J=7.9 Hz, 1H), 6.95-6.91 (m, 1H), 6.64 (dd, J=8.2, 1.9 Hz, 1H), 4.23 (q, J=7.1 Hz, 2H), 3.54 (q, J=6.7 Hz, 2H), 3.26-3.18 (m, 4H), 2.74 (t, J=6.9 Hz, 2H), 2.53 (s, 3H), 2.00-1.90 (m, 4H), 1.27 (t, J=7.1 Hz, 3H).

### Example 3: Ethyl 2-(3-(3-(1 H-pyrazol-1-yl)benzamido)propanamido)-4-methylthiazole-5-carboxylate

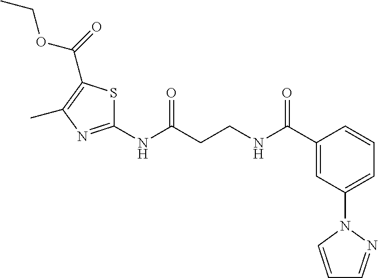 [SMILES: CCOC(=O)c1sc(NC(=O)CCNC(=O)c2cccc(-n3cccn3)c2)nc1C]

27 mg (51%, 0.0632 mmol) colorless solid. HPLC/MS m/z: 428.1 [M+H]⁺, Rt (S): 2.70 min. ¹H NMR (500 MHz, DMSO-d₆) δ 12.53 (s, 1H), 8.78 (t, J=5.5 Hz, 1H), 8.56-8.52 (m, 1H), 8.26 (t, J=1.8 Hz, 1H), 7.98 (ddd, J=8.1, 2.2, 0.9 Hz, 1H), 7.78 (d, J=1.4 Hz, 1H), 7.74 (dt, J=7.7, 1.1 Hz, 1H), 7.58 (t, J=7.9 Hz, 1H), 6.57 (dd, J=2.5, 1.8 Hz, 1H), 4.23 (q, J=7.1 Hz, 2H), 3.60 (q, J=6.7 Hz, 2H), 2.78 (t, J=6.8 Hz, 2H), 2.53 (s, 3H), 1.27 (t, J=7.1 Hz, 3H).

### Example 4: Ethyl 4-methyl-2-(3-(3-(5-methyl-1,2,4-oxadiazol-3-yl)benzamido)propanamido)thiazole-5-carboxylate

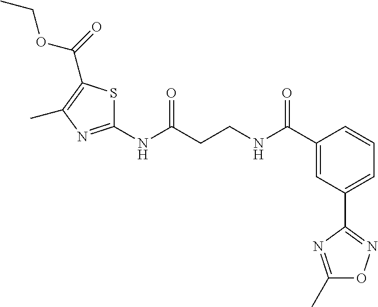 [SMILES: CCOC(=O)c1sc(NC(=O)CCNC(=O)c2cccc(-c3noc(C)n3)c2)nc1C]

47 mg(68%, 0.1060 mmol) off-white solid. HPLC/MS m/z: 444.1 [M+H]⁺, Rt (S): 2.77 min. ¹H NMR (500 MHz, DMSO-d₆) δ 12.53 (s, 1H), 8.78 (t, J=5.5 Hz, 1H), 8.56-8.52 (m, 1H), 8.26 (t, J=1.8 Hz, 1H), 7.98 (ddd, J=8.1, 2.2, 0.9 Hz, 1H), 7.78 (d, J=1.4 Hz, 1H), 7.74 (dt, J=7.7, 1.1 Hz, 1H), 7.58 (t, J=7.9 Hz, 1H), 6.57 (dd, J=2.5, 1.8 Hz, 1H), 4.23 (q, J=7.1 Hz, 2H), 3.60 (q, J=6.7 Hz, 2H), 2.78 (t, J=6.8 Hz, 2H), 2.53 (s, 3H), 1.27 (t, J=7.1 Hz, 3H).

### Example 5: Ethyl 4-methyl-2-(3-(5,6,7,8-tetrahydronaphthalene-2-carboxamido)propanamido)thiazole-5-carboxylate

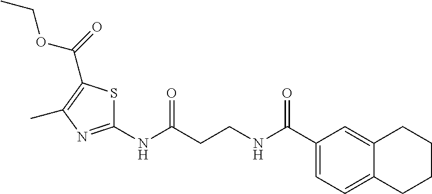 [SMILES: CCOC(=O)c1sc(NC(=O)CCNC(=O)c2ccc3c(c2)CCCC3)nc1C]

24 mg (37%, 0.0578 mmol) colorless solid. HPLC/MS m/z: 416.2 [M+H]⁺, Rt (S): 3.14 min. ¹H NMR (500 MHz, DMSO-d₆) δ 12.50 (s, 1H), 8.46 (t, J=5.5 Hz, 1H), 7.52 (d, J=7.2 Hz, 2H), 7.10 (d, J=8.2 Hz, 1H), 4.23 (q, J=7.1 Hz, 2H), 3.54 (q, J=6.8 Hz, 2H), 2.74 (t, J=6.8 Hz, 6H), 2.53 (s, 3H), 1.73 (quint J=3.4 Hz, 4H), 1.27 (t, J=7.1 Hz, 3H).

### Example 6: Ethyl 4-methyl-2-(3-(3-(oxazol-5-yl)benzamido)propanamido)thiazole-5-carboxylate

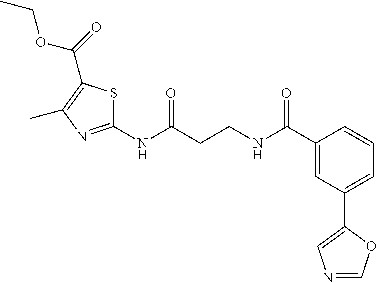 [SMILES: CCOC(=O)c1sc(NC(=O)CCNC(=O)c2cccc(-c3cnco3)c2)nc1C]

40 mg (60%, 0.0934 mmol) yellow solid. HPLC/MS m/z: 429.1 [M+H]⁺, Rt (S): 2.67 min. ¹H NMR (500 MHz, DMSO-d₆) δ 12.54 (s, 1H), 8.77 (t, J=5.5 Hz, 1H), 8.50 (s, 1H), 8.16 (t, J=1.6 Hz, 1H), 7.93-7.85 (m, 1H), 7.83-7.81 (m, 1H), 7.81-7.80 (m, 1H), 7.75 (s, 1H), 7.57 (t, J=7.7 Hz, 1H), 4.23 (q, J=7.1 Hz, 2H), 3.59 (q, J=6.7 Hz, 2H), 2.78 (t, J=6.8 Hz, 2H), 2.53 (s, 3H), 1.27 (t, J=7.1 Hz, 3H).

### Example 7: Ethyl 4-methyl-2-(3-(3-morpholinobenzamido)propanamido)thiazole-5-carboxylate

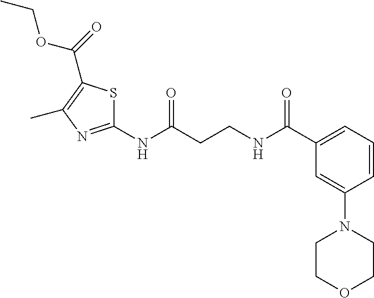 [SMILES: CCOC(=O)c1sc(NC(=O)CCNC(=O)c2cccc(N3CCOCC3)c2)nc1C]

22 mg (32%, 0.0493 mmol) off-white solid. HPLC/MS m/z: 447.2 [M+H]⁺, Rt (S): 2.67 min. ¹H NMR (500 MHz, DMSO-d₆) δ 12.52 (s, 1H), 8.54 (t, J=5.6 Hz, 1H), 7.35-7.32 (m, 1H), 7.29 (t, J=7.8 Hz, 1H), 7.25 (d, J=7.7 Hz, 1H), 7.08 (ddd, J=8.1, 2.5, 1.1 Hz, 1H), 4.24 (q, J=7.1 Hz, 2H), 3.80-3.69 (m, 4H), 3.55 (q, J=6.7 Hz, 2H), 3.17-3.07 (m, 4H), 2.75 (t, J=6.8 Hz, 2H), 2.53 (s, 3H), 1.28 (t, J=7.1 Hz, 3H).

### Example 8: Ethyl (S)-2-(2-hydroxy-3-(3-methylbenzamido)propanamido)-4-methylthiazole-5-carboxylate

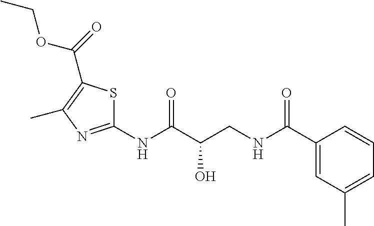 [SMILES: CCOC(=O)c1sc(NC(=O)[C@@H](O)CNC(=O)c2cccc(C)c2)nc1C]

Using (S)-3-((tert-butoxycarbonyl)amino)-2-hydroxypropanoic acid to afford 16 mg (12%, 0.041 mmol) of ethyl (S)-2-(2-hydroxy-3-(3-methylbenzamido)propanamido)-4-methylthiazole-5-carboxylate as a colorless solid. HPLC/MS m/z: 392.1 [M+H]⁺, Rt (S): 2.69 min. ¹H NMR (500 MHz, DMSO-d₆) δ 12.13 (s, 1H), 8.46 (t, J=5.8 Hz, 1H), 7.62 (quint, J=1.0 Hz, 1H), 7.61-7.57 (m, 1H), 7.33 (dd, J=5.0, 0.9 Hz, 2H), 5.96 (s, 1H), 4.40 (s, 1H), 4.24 (q, J=7.1 Hz, 2H), 3.56 (s, 2H), 2.55 (s, 3H), 2.34 (s, 3H), 1.28 (t, J=7.1 Hz, 3H).

### Example 9: Ethyl 2-[3-[(3-methoxycarbonylbenzoyl)amino]propanoylamino]-4-methyl-thiazole-5-carboxylate

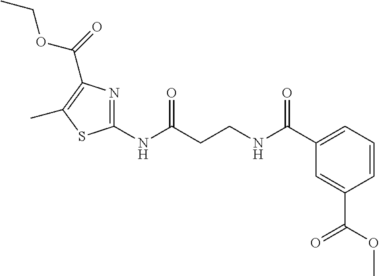 [SMILES: CCOC(=O)c1nc(NC(=O)CCNC(=O)c2cccc(C(=O)OC)c2)sc1C]

3-Methoxycarbonylbenzoic acid (35 mg, 0.19 mmol), ethyl 2-(3-aminopropanoyl-amino)-4-methyl-thiazole-5-carboxylate (50 mg, 0.19 mmol) and DIPEA (103 uL, 0.58 mmol) were dissolved in DMF (1.94 mL). HATU (215.17 mg, 0.9100 mmol) was added, and the reaction mixture was stirred over the weekend at rt. The reaction mixture was then diluted with EtOAc (40 mL) and washed with water (40 mL). The aqueous layer was re-extracted with EtOAc (40 mL). The organic layers were combined and washed with aqueous saturated sodium bicarbonate solution (50 mL) and brine (50 mL). The organic layer was dried (MgSO₄), filtered and concentrated in vacuo. The crude was purified using reverse phase column chromatography with a 12 g C18 Biotage ULTRA column. The column was eluted with 30-100% MeOH in water (+0.1% formic acid modifier in both) to give 16 mg (20%, 0.04 mmol) of ethyl 2-[3-[(3-methoxycarbonylbenzoyl)amino]propanoylamino]-4-methyl-thiazole-5-carboxylate as a colorless fluffy solid. HPLC/MS m/z: 420.07 [M+H]⁺, Rt (R): 1.29 min. ¹H NMR (500 MHz, DMSO-d₆) δ 12.53 (s, 1H), 8.85 (t, J=5.5 Hz, 1H), 8.42 (t, J=1.8 Hz, 1H), 8.09 (dd, J=7.8, 1.8 Hz, 2H), 7.62 (t, J=7.8 Hz, 1H), 4.23 (q, J=7.1 Hz, 2H), 3.88 (s, 3H), 3.59 (q, J=6.6 Hz, 2H), 2.77 (t, J=6.8 Hz, 2H), 2.53 (s, 3H), 1.27 (t, J=7.1 Hz, 3H).

The following examples were prepared in a similar manner:

### Example 10: Ethyl 2-[3-[(3-fluoro-5-methoxycarbonyl-benzoyl)amino]propanoylamino]-4-methyl-thiazole-5-carboxylate

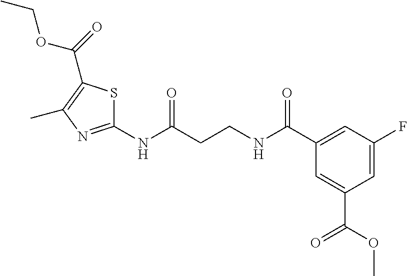 [SMILES: CCOC(=O)c1sc(NC(=O)CCNC(=O)c2cc(F)cc(C(=O)OC)c2)nc1C]

43 mg (51%) colorless, fluffy solid. HPLC/MS m/z: 438.11 [M+H]⁺, Rt (R): 1.35 min. ¹H NMR (500 MHz, DMSO-d₆) δ 12.54 (s, 1H), 8.93 (t, J=5.5 Hz, 1H), 8.28 (t, J=1.5 Hz, 1H), 7.92 (ddd, J=9.4, 2.6, 1.5 Hz, 1H), 7.85 (ddd, J=8.8, 2.6, 1.4 Hz, 1H), 4.23 (q, J=7.1 Hz, 2H), 3.89 (s, 3H), 3.66-3.47 (m, 2H), 2.77 (t, J=6.8 Hz, 2H), 2.53 (s, 3H), 1.27 (t, J=7.1 Hz, 3H).

### Example 11: Ethyl 2-[3-[(2-fluoro-3-methoxycarbonyl-benzoyl)amino]propanoylamino]-4-methyl-thiazole-5-carboxylate

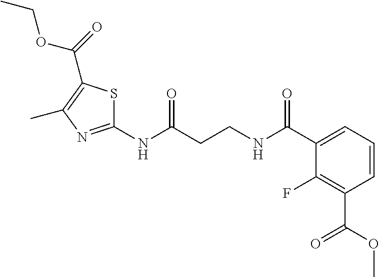 [SMILES: CCOC(=O)c1sc(NC(=O)CCNC(=O)c2cccc(C(=O)OC)c2F)nc1C]

26 mg (31%) colorless, fluffy solid. HPLC/MS m/z: 438.11 [M+H]⁺, Rt (R): 1.23 min. ¹H NMR (500 MHz, DMSO-d₆) δ 12.52 (s, 1H), 8.62 (t, J=5.4 Hz, 1H), 7.95 (ddd, J=7.7, 6.8, 1.9 Hz, 1H), 7.78 (ddd, J=8.0, 6.3, 1.9 Hz, 1H), 7.38 (t, J=7.7 Hz, 1H), 4.24 (q, J=7.1 Hz, 2H), 3.86 (s, 3H), 3.57 (q, J=6.5 Hz, 2H), 2.75 (t, J=6.8 Hz, 2H), 2.54 (s, 3H), 1.28 (t, J=7.1 Hz, 3H).

### Example 12: Ethyl 2-[3-[(2-chloro-5-methoxycarbonyl-benzoyl)amino]propanoylamino]-4-methyl-thiazole-5-carboxylate

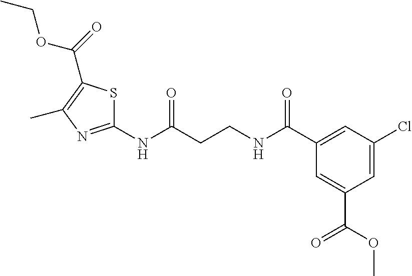 [SMILES: CCOC(=O)c1sc(NC(=O)CCNC(=O)c2cc(Cl)cc(C(=O)OC)c2)nc1C]

41 mg (46%) fine, colorless powder. HPLC/MS m/z: 454.08 [M+H]⁺, Rt (R): 1.27 min. ¹H NMR (500 MHz, DMSO-d₆) δ 12.53 (s, 1H), 8.72 (t, J=5.6 Hz, 1H), 7.96 (dd, J=8.4, 2.2 Hz, 1H), 7.91 (d, J=2.1 Hz, 1H), 7.64 (d, J=8.4 Hz, 1H), 4.24 (q, J=7.1 Hz, 2H), 3.86 (s, 3H), 3.56 (q, J=6.4 Hz, 2H), 2.77 (t, J=6.7 Hz, 2H), 2.54 (s, 3H), 1.28 (t, J=7.1 Hz, 3H).

### Example 13: Ethyl 2-[3-[(3-chloro-5-methoxycarbonyl-benzoyl)amino]propanoylamino]-4-methyl-thiazole-5-carboxylate

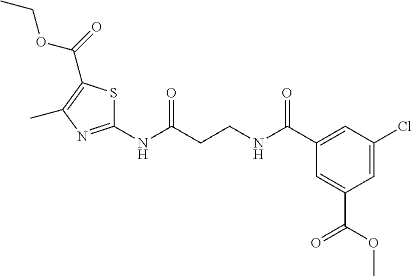 [SMILES: CCOC(=O)c1sc(NC(=O)CCNC(=O)c2cc(Cl)cc(C(=O)OC)c2)nc1C]

37 mg (42%) colorless, granular solid. HPLC/MS m/z: 454.08 [M+H]⁺, Rt (R): 1.41 min. ¹H NMR (500 MHz, DMSO-d₆) δ 12.53 (s, 1H), 8.95 (t, J=5.5 Hz, 1H), 8.35 (t, J=1.5 Hz, 1H), 8.14 (dd, J=2.1, 1.6 Hz, 1H), 8.06 (dd, J=2.1, 1.5 Hz, 1H), 4.23 (q, J=7.1 Hz, 2H), 3.90 (s, 3H), 3.59 (q, J=6.5 Hz, 2H), 2.77 (t, J=6.8 Hz, 2H), 2.53 (s, 3H), 1.27 (t, J=7.1 Hz, 3H).

### Example 14: Ethyl 2-[3-[(3-methoxycarbonyl-4-methyl-benzoyl)amino]propanoylamino]-4-methyl-thiazole-5-carboxylate

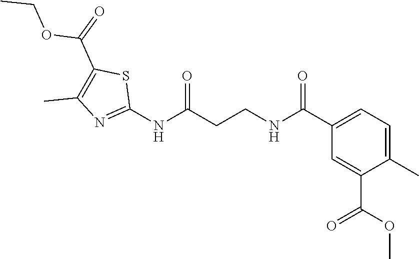 [SMILES: CCOC(=O)c1sc(NC(=O)CCNC(=O)c2ccc(C)c(C(=O)OC)c2)nc1C]

56 mg (66%) colorless solid. HPLC/MS m/z: 434.14 [M+H]⁺, Rt (R): 1.33 min. ¹H NMR (500 MHz, DMSO-d₆) δ 12.52 (s, 1H), 8.73 (t, J=5.5 Hz, 1H), 8.28 (d, J=2.0 Hz, 1H), 7.92 (dd, J=7.9, 2.0 Hz, 1H), 7.42 (d, J=8.0 Hz, 1H), 4.23 (q, J=7.1 Hz, 2H), 3.85 (s, 3H), 3.57 (q, J=6.7 Hz, 2H), 2.76 (t, J=6.8 Hz, 2H), 2.55 (s, 3H), 2.53 (s, 3H), 1.27 (t, J=7.1 Hz, 3H).

### Example 15: Ethyl 2-[3-[(3-methoxycarbonyl-5-methyl-benzoyl)amino]propanoylamino]-4-methyl-thiazole-5-carboxylate

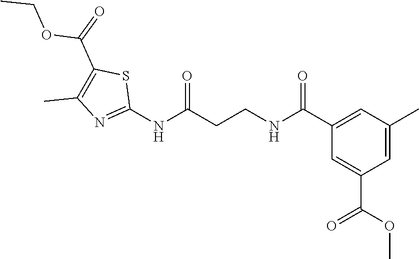 [SMILES: CCOC(=O)c1sc(NC(=O)CCNC(=O)c2cc(C)cc(C(=O)OC)c2)nc1C]

56 mg (67%) colorless solid. HPLC/MS m/z: 434.14 [M+H]⁺, Rt (R): 1.35 min. ¹H NMR (500 MHz, DMSO-d₆) δ 12.53 (s, 1H), 8.78 (t, J=5.5 Hz, 1H), 8.30-8.10 (m, 1H), 7.91 (dd, J=1.6, 0.8 Hz, 2H), 4.24 (q, J=7.1 Hz, 2H), 3.87 (s, 3H), 3.57 (q, J=6.5 Hz, 2H), 2.76 (t, J=6.8 Hz, 2H), 2.54 (s, 3H), 2.41 (s, 3H), 1.27 (t, J=7.1 Hz, 3H).

### Example 16: Ethyl 2-[3-[(3-methoxy-5-methoxycarbonyl-benzoyl)amino]propanoylamino]-4-methyl-thiazole-5-carboxylate

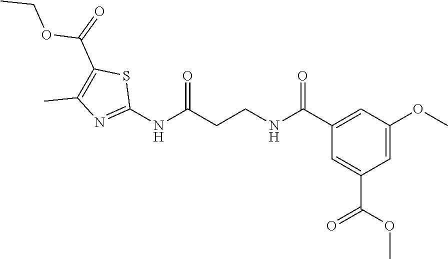 [SMILES: CCOC(=O)c1sc(NC(=O)CCNC(=O)c2cc(OC)cc(C(=O)OC)c2)nc1C]

35 mg (40%) colorless solid. HPLC/MS m/z: 450.13 [M+H]⁺, Rt (R): 1.33 min. ¹H NMR (500 MHz, DMSO-d₆) δ 12.51 (s, 1H), 8.81 (t, J=5.5 Hz, 1H), 8.02 (t, J=1.4 Hz, 1H), 7.64 (dd, J=2.6, 1.5 Hz, 1H), 7.57 (dd, J=2.6, 1.4 Hz, 1H), 4.24 (q, J=7.1 Hz, 2H), 3.88 (s, 3H), 3.86 (s, 3H), 3.58 (q, J=6.6 Hz, 2H), 2.77 (t, J=6.8 Hz, 2H), 2.53 (s, 3H), 1.28 (t, J=7.1 Hz, 3H).

### Example 17: Ethyl 2-[3-[(3-cyano-5-methoxycarbonyl-benzoyl)amino]propanoylamino]-4-methyl-thiazole-5-carboxylate

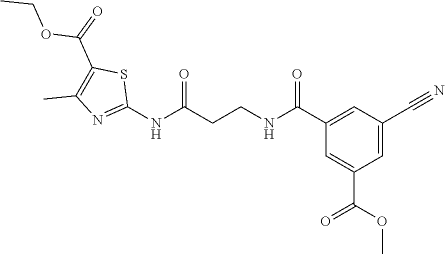 [SMILES: CCOC(=O)c1sc(NC(=O)CCNC(=O)c2cc(C#N)cc(C(=O)OC)c2)nc1C]

32 mg (37%) colorless, amorphous solid. HPLC/MS m/z: 445.12 [M+H]⁺, Rt (R): 1.30 min. ¹H NMR (500 MHz, DMSO-d₆) δ 12.54 (s, 1H), 9.02 (t, J=5.5 Hz, 1H), 8.64 (t, J=1.7 Hz, 1H), 8.50 (t, J=1.6 Hz, 1H), 8.47 (t, J=1.6 Hz, 1H), 4.23 (q, J=7.1 Hz, 2H), 3.92 (s, 3H), 3.60 (q, J=6.4 Hz, 2H), 3.32 (s, 4H), 2.78 (t, J=6.7 Hz, 2H), 2.53 (s, 3H), 1.27 (t, J=7.1 Hz, 3H).

### Example 18: Ethyl 2-[3-[(3-bromo-5-methoxycarbonyl-benzoyl)amino]propanoylamino]-4-methyl-thiazole-5-carboxylate

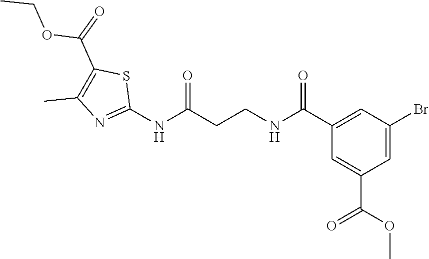 [SMILES: CCOC(=O)c1sc(NC(=O)CCNC(=O)c2cc(Br)cc(C(=O)OC)c2)nc1C]

163 mg (47%) amorphous, colorless solid. HPLC/MS m/z: 498.03 [M+H]⁺, Rt (R): 1.43 min. ¹H NMR (500 MHz, DMSO-d₆) δ 12.54 (s, 1H), 8.96 (t, J=5.5 Hz, 1H), 8.39 (s, 1H), 8.27 (t, J=1.8 Hz, 1H), 8.18 (t, J=1.7 Hz, 1H), 4.23 (q, J=7.1 Hz, 2H), 3.89 (s, 3H), 3.58 (q, J=6.4 Hz, 2H), 2.76 (q, J=5.7, 4.7 Hz, 2H), 2.53 (s, 3H), 1.27 (t, J=7.1 Hz, 3H).

### Example 19: Ethyl 2-[3-[(3-ethyl-5-methoxycarbonyl-benzoyl)amino]-propanoylamino]-4-methyl-thiazole-5-carboxylate

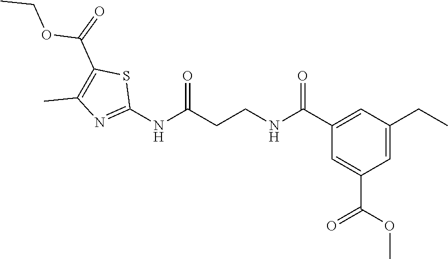 [SMILES: CCOC(=O)c1sc(NC(=O)CCNC(=O)c2cc(CC)cc(C(=O)OC)c2)nc1C]

3 mg (7%) colorless, amorphous solid. HPLC/MS m/z: 448.15 [M+H]⁺, Rt (R): 1.40 min. ¹H NMR (500 MHz, MeOD-d₄) δ 8.28 (t, J=1.7 Hz, 1H), 8.03-7.99 (m, 1H), 7.90 (dt, J=2.3, 1.1 Hz, 1H), 4.31 (q, J=7.1 Hz, 2H), 3.93 (s, 3H), 3.76 (t, J=6.7 Hz, 2H), 2.85 (t, J=6.7 Hz, 2H), 2.77 (q, J=7.6 Hz, 2H), 2.58 (s, 3H), 1.36 (t, J=7.1 Hz, 3H), 1.28 (t, J=7.6 Hz, 3H).

### Example 20: Ethyl 2-[3-[(3-hydroxy-5-methoxycarbonyl-benzoyl)amino]-propanoylamino]-4-methyl-thiazole-5-carboxylate

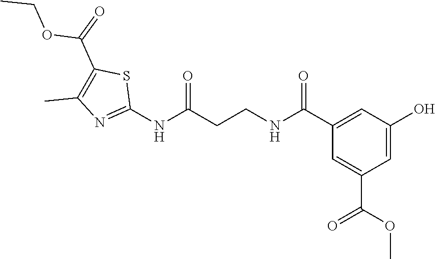 [SMILES: CCOC(=O)c1sc(NC(=O)CCNC(=O)c2cc(O)cc(C(=O)OC)c2)nc1C]

20 mg (12%) colorless, amorphous solid. HPLC/MS m/z: 436.12 [M+H]⁺, Rt (R): 1.26 min. ¹H NMR (500 MHz, DMSO-d₆) δ 12.52 (s, 1H), 10.12 (s, 1H), 8.73 (t, J=5.6 Hz, 1H), 7.86 (t, J=1.6 Hz, 1H), 7.53-7.34 (m, 2H), 4.24 (q, J=7.1 Hz, 2H), 3.85 (s, 3H), 3.55 (q, J=6.5 Hz, 2H), 2.75 (t, J=6.8 Hz, 2H), 2.54 (s, 3H), 1.28 (t, J=7.1 Hz, 3H).

### Example 21: Ethyl 4-methyl-2-[3-[[3-(5-methyl-1,2,4-oxadiazol-3-yl)benzoyl]amino]-propylamino]thiazole-5-carboxylate

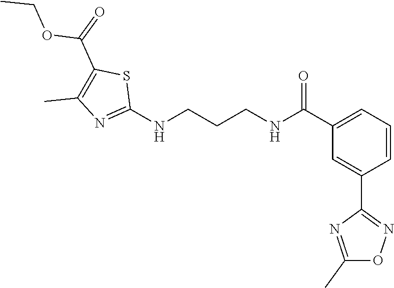 [SMILES: CCOC(=O)c1sc(NCCCNC(=O)c2cccc(-c3noc(C)n3)c2)nc1C]

50 mg (51%) colorless, amorphous solid. HPLC/MS m/z: 430.15 [M+H]⁺, Rt (R): 1.31 min. ¹H NMR (500 MHz, DMSO-d₆) δ 8.74 (t, J=5.6 Hz, 1H), 8.47 (dt, J=1.8, 1.0 Hz, 1H), 8.36 (t, J=5.5 Hz, 1H), 8.16-8.10 (m, 1H), 8.04 (ddd, J=7.8, 1.8, 1.2 Hz, 1H), 7.70-7.61 (m, 1H), 4.14 (q, J=7.1 Hz, 2H), 3.33 (m, 4H, plus H₂O peak), 2.69 (s, 3H), 2.39 (s, 3H), 1.83 (quint, J=6.9 Hz, 2H), 1.21 (t, J=7.1 Hz, 3H).

### Example 22: Ethyl 2-[3-[[3-methoxycarbonyl-5-[(E)-styryl]benzoyl]amino]-propanoylamino]-4-methyl-thiazole-5-carboxylate

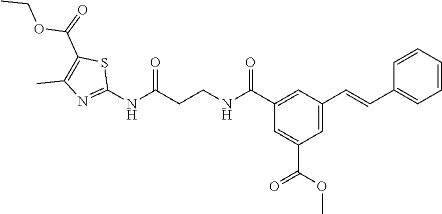 [SMILES: CCOC(=O)c1sc(NC(=O)CCNC(=O)c2cc(/C=C/c3ccccc3)cc(C(=O)OC)c2)nc1C]

15 mg (24%) colorless, amorphous solid. HPLC/MS m/z: 522.17 [M+H]⁺, Rt (R): 1.56 min. ¹H NMR (500 MHz, DMSO-d₆) δ 12.55 (s, 1H), 8.90 (t, J=5.5 Hz, 1H), 8.34 (t, J=1.7 Hz, 1H), 8.29 (t, J=1.6 Hz, 1H), 8.27 (t, J=1.7 Hz, 1H), 7.72-7.59 (m, 2H), 7.48-7.37 (m, 4H), 7.31 (t, J=7.4 Hz, 1H), 4.23 (q, J=7.1 Hz, 2H), 3.91 (s, 3H), 3.62 (q, J=6.4 Hz, 2H), 2.79 (t, J=6.8 Hz, 2H), 2.53 (s, 3H), 1.27 (t, J=7.1 Hz, 3H).

### Example 23: Ethyl 2-(3-(3-(1,2,4-oxadiazol-3-yl)benzamido)propanamido)-4-methylthiazole-5-carboxylate

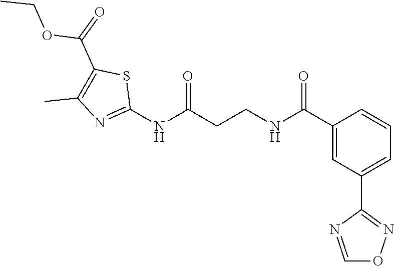 [SMILES: CCOC(=O)c1sc(NC(=O)CCNC(=O)c2cccc(-c3ncon3)c2)nc1C]

51 mg (75%). HPLC/MS m/z: 430.1 [M+H]⁺, Rt (U): 2.77 min. ¹H NMR (600 MHz, DMSO-d₆) δ 12.53 (s, 1H), 9.76 (s, 1H), 8.87 (t, J=5.5 Hz, 1H), 8.50 (t, J=1.7 Hz, 1H), 8.18 (dt, J=7.7, 1.4 Hz, 1H), 8.07-8.02 (m, 1H), 7.68 (t, J=7.8 Hz, 1H), 4.23 (q, J=7.1 Hz, 2H), 3.60 (q, J=6.6 Hz, 2H), 2.78 (t, J=6.8 Hz, 2H), 2.53 (s, 3H), 1.27 (t, J=7.1 Hz, 3H).

### Example 24: Ethyl 2-(3-(3-chloro-5-(5-methyl-1,2,4-oxadiazol-3-yl)benzamido)-propanamido)-4-methylthiazole-5-carboxylate

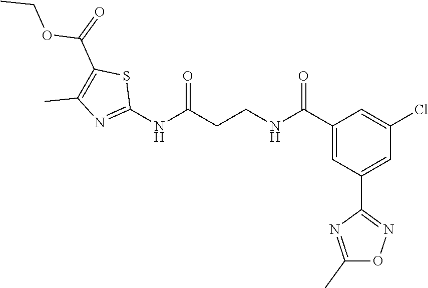 [SMILES: CCOC(=O)c1sc(NC(=O)CCNC(=O)c2cc(Cl)cc(-c3noc(C)n3)c2)nc1C]

Example 24.1.: Methyl 3-chloro-5-cyano-benzoate (250.00 mg, 1.2781 mmol) was dissolved in MeOH (12.78 mL). Hydroxylamine (50% in water) (0.20 mL, 6.3906 mmol) was added, and the solution was stirred at rt for overnight. Solvent was removed to afford 264 mg (90%, 1.1547 mmol) of methyl 3-chloro-5-[(Z)-N′-hydroxycarbamimidoyl]benzoate as a brown oil which was used without further purification. HPLC/MS m/z: 229.04, [M+H]⁺, Rt (P): 0.59 min.

Example 24.2.: Acetic acid (0.06 mL, 1.0852 mmol), methyl 3-chloro-5-[(Z)-N′-hydroxycarbamimidoyl]benzoate (175.00 mg, 0.7654 mmol) and EDC.HCl (207.37 mg, 1.0818 mmol) were dissolved in THF (4.53 mL) and MeCN (4.53 mL) in a microwave vial under N₂. The reaction mixture was stirred overnight at rt. DIPEA (0.38 mL, 2.1652 mmol) was added and the solution was heated under microwave irradiation for 30 min at 150° C. Solvents were evaporated and EtOAc was added.

The residue was washed twice with HCl 0.1 N, one time with a saturated solution of NaHCO₃, brine and dried over MgSO₄. Purification by silica gel column chromatography (eluent: 0-4% of EtOAc in cyclohexane) afforded 50 mg (26%, 0.1979 mmol) of methyl 3-chloro-5-(5-methyl-1,2,4-oxadiazol-3-yl)benzoate as a beige oil. HPLC/MS m/z: 253.04, [M+H]⁺, Rt (R): 1.54 min.

Example 24.3: To methyl 3-chloro-5-(5-methyl-1,2,4-oxadiazol-3-yl)benzoate (50.00 mg, 0.1979 mmol) in THF (0.99 mL) was added water (0.99 mL) followed by LiOH hydrate (24.91 mg, 0.5937 mmol). After stirring for at rt volatile were removed in vacuo and the crude partitioned between EtOAc (15 mL) and 1 M HCl (15 mL). The organic was retained and the aqueous extracted with further EtOAc (10 mL). The organics were combined, dried over MgSO₄ and evaporated to dryness to give 46 mg (97%, 0.1928 mmol) of 3-chloro-5-(5-methyl-1,2,4-oxadiazol-3-yl)benzoic acid as a colorless powder. Used in the next step without further purification. HPLC/MS m/z: 239.02, [M+H]⁺, Rt (R): 1.44 min.

Example 24.4.: An analogous procedure to the preparation of 0 using 3-chloro-5-(5-methyl-1,2,4-oxadiazol-3-yl)benzoic acid and ethyl 2-(3-aminopropanoylamino)-4-methyl-thiazole-5-carboxylate afforded 26 mg (52%, 0.0544 mmol) of ethyl 2-(3-(3-chloro-5-(5-methyl-1,2,4-oxadiazol-3-yl)benzamido)propanamido)-4-methylthiazole-5-carboxylate as a colorless solid. HPLC/MS m/z: 478.09, [M+H]⁺, Rt (S): 3.06 min. ¹H NMR (500 MHz, DMSO-d₆) δ 12.54 (s, 1H), 8.98 (t, J=5.5 Hz, 1H), 8.40 (t, J=1.6 Hz, 1H), 8.08 (dt, J=9.4, 1.9 Hz, 2H), 4.23 (q, J=7.1 Hz, 2H), 3.60 (q, J=6.4 Hz, 2H), 2.78 (t, J=6.8 Hz, 2H), 2.69 (s, 3H), 2.53 (s, 3H), 1.27 (t, J=7.1 Hz, 3H).

The following examples were prepared in an analogous manner using propyl 2-amino-4-methyl-thiazole-5-carboxylate.

### Example 25: Propyl 2-(3-(3-chloro-5-(5-methyl-1,2,4-oxadiazol-3-yl)benzamido)propanamido)-4-methylthiazole-5-carboxylate

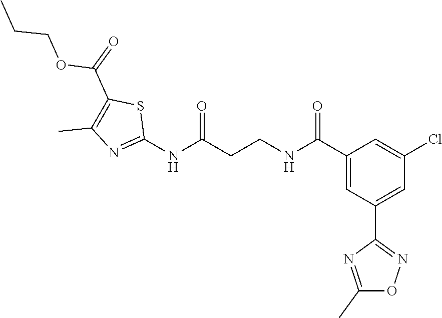 [SMILES: CCCOC(=O)c1sc(NC(=O)CCNC(=O)c2cc(Cl)cc(-c3noc(C)n3)c2)nc1C]

31 mg (60%) colorless solid. HPLC/MS m/z: 492.11, [M+H]⁺, Rt (S): 3.20 min. ¹H NMR (500 MHz, DMSO-d₆) δ 12.55 (s, 1H), 8.98 (t, J=5.6 Hz, 1H), 8.40 (t, J=1.5 Hz, 1H), 8.08 (dt, J=7.6, 1.9 Hz, 2H), 4.15 (t, J=6.5 Hz, 2H), 3.60 (q, J=6.5 Hz, 2H), 2.78 (t, J=6.8 Hz, 2H), 2.69 (s, 3H), 2.53 (s, 3H), 1.67 (sext, J=7.1 Hz, 2H), 0.93 (t, J=7.4 Hz, 3H).

### Example 26: Propyl 4-methyl-2-(3-(3-methyl-5-(5-methyl-1,2,4-oxadiazol-3-yl)benzamido)propanamido)thiazole-5-carboxylate

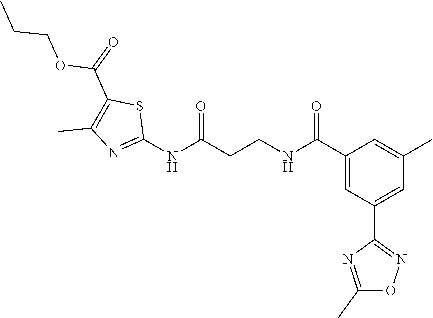 [SMILES: CCCOC(=O)c1sc(NC(=O)CCNC(=O)c2cc(C)cc(-c3noc(C)n3)c2)nc1C]

Prepared in analogous procedure from methyl 3-cyano-5-methyl-benzoate to afford 58 mg (73%) of propyl 4-methyl-2-(3-(3-methyl-5-(5-methyl-1,2,4-oxadiazol-3-yl)benzamido)propanamido)thiazole-5-carboxylate as a colorless solid. HPLC/MS m/z: 472.16, [M+H]⁺, Rt (S): 3.04 min. ¹H NMR (500 MHz, DMSO-d₆) δ 12.54 (s, 1H), 8.80 (t, J=5.5 Hz, 1H), 8.25 (t, J=1.6 Hz, 1H), 7.95 (td, J=1.6, 0.8 Hz, 1H), 7.85 (td, J=1.7, 0.8 Hz, 1H), 4.15 (t, J=6.5 Hz, 2H), 3.59 (q, J=6.5 Hz, 2H), 2.78 (t, J=6.8 Hz, 2H), 2.67 (s, 3H), 2.53 (s, 3H), 2.44-2.41 (m, 3H), 1.67 (dtd, J=13.8, 7.4, 6.4 Hz, 2H), 0.94 (t, J=7.4 Hz, 3H).

### Example 27: Propyl 1-methyl-3-(3-(3-methyl-5-(5-methyl-1,2,4-oxadiazol-3-yl)benzamido)propanamido)-1 H-pyrazole-5-carboxylate

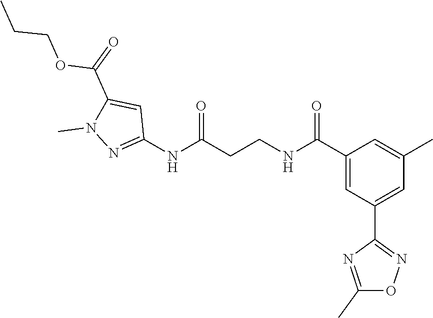 [SMILES: CCCOC(=O)c1cc(NC(=O)CCNC(=O)c2cc(C)cc(-c3noc(C)n3)c2)nn1C]

Propyl 1-methyl-3-(3-(3-methyl-5-(5-methyl-1,2,4-oxadiazol-3-yl)benzamido)-propanamido)-1 H-pyrazole-5-carboxylate (20 mg, 21%, 0.0440 mmol) was prepared in analogous procedure from methyl 3-cyano-5-methyl-benzoate and propyl 5-amino-2-methyl-pyrazole-3-carboxylate as a colorless solid. HPLC/MS m/z: 455.20, [M+H]⁺, Rt (S): 2.86 min. ¹H NMR (500 MHz, DMSO-d₆) δ 10.68 (s, 1H), 8.75 (t, J=5.5 Hz, 1H), 8.26 (d, J=1.7 Hz, 1H), 7.95 (td, J=1.7, 0.8 Hz, 1H), 7.86 (td, J=1.7, 0.8 Hz, 1H), 7.05 (s, 1H), 4.20 (t, J=6.5 Hz, 2H), 3.98 (s, 3H), 3.54 (td, J=7.0, 5.4 Hz, 2H), 2.67 (s, 3H), 2.62 (t, J=7.1 Hz, 2H), 2.43 (s, 3H), 1.76-1.65 (m, 2H), 0.94 (t, J=7.4 Hz, 3H).

### Example 28: Propyl 2-(3-(3-cyano-5-(trifluoromethyl)benzamido)propanamido)-4-methylthiazole-5-carboxylate

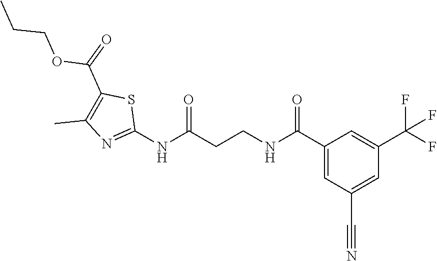 [SMILES: CCCOC(=O)c1sc(NC(=O)CCNC(=O)c2cc(C#N)cc(C(F)(F)F)c2)nc1C]

40.5 mg (53%) yellow solid. HPLC/MS m/z: 469.12, [M+H]⁺, Rt (S): 3.09 min. ¹H NMR (500 MHz, DMSO-d₆) δ 12.55 (s, 1H), 9.04 (t, J=5.5 Hz, 1H), 8.54 (d, J=1.8 Hz, 2H), 8.43 (s, 1H), 4.15 (t, J=6.5 Hz, 2H), 3.61 (q, J=6.4 Hz, 2H), 2.78 (t, J=6.7 Hz, 2H), 2.54 (s, 3H), 1.67 (h, J=7.0 Hz, 2H), 0.94 (t, J=7.4 Hz, 3H).

### Example 29: Propyl 2-(3-(3-(tert-butyl)-5-(methoxycarbonyl)benzamido)propanamido)-4-methylthiazole-5-carboxylate

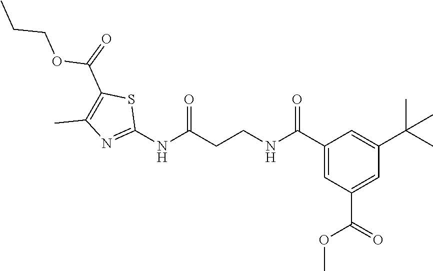 [SMILES: CCCOC(=O)c1sc(NC(=O)CCNC(=O)c2cc(C(=O)OC)cc(C(C)(C)C)c2)nc1C]

23 mg (36%) colorless solid. HPLC/MS m/z: 490.20, [M+H]⁺, Rt (R): 3.31 min. ¹H NMR (500 MHz, DMSO-d₆) δ 12.54 (s, 1H), 8.86 (t, J=5.6 Hz, 1H), 8.25 (t, J=1.6 Hz, 1H), 8.11 (t, J=1.8 Hz, 1H), 8.07 (t, J=1.7 Hz, 1H), 4.15 (t, J=6.5 Hz, 2H), 3.88 (s, 3H), 3.59 (q, J=6.5 Hz, 2H), 2.77 (t, J=6.8 Hz, 2H), 2.53 (s, 3H), 1.67 (dtd, J=13.7, 7.4, 6.5 Hz, 2H), 1.32 (s, 9H), 0.94 (t, J=7.4 Hz, 3H).

### Example 30: Propyl 2-(3-(3-cyano-5-(methoxycarbonyl)benzamido)propanamido)-4-methylthiazole-5-carboxylate

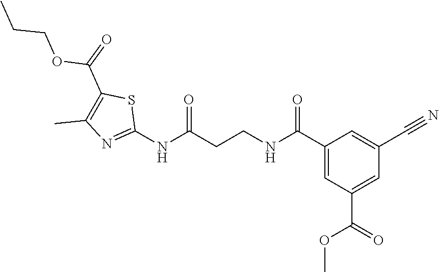 [SMILES: CCCOC(=O)c1sc(NC(=O)CCNC(=O)c2cc(C#N)cc(C(=O)OC)c2)nc1C]

15 mg (10%) colorless solid. HPLC/MS m/z: 459.13, [M+H]⁺, Rt (S): 2.94 min. ¹H NMR (500 MHz, DMSO-d₆) δ 12.54 (s, 1H), 9.02 (t, J=5.5 Hz, 1H), 8.64 (t, J=1.7 Hz, 1H), 8.50 (t, J=1.7 Hz, 1H), 8.47 (t, J=1.6 Hz, 1H), 4.15 (t, J=6.5 Hz, 2H), 3.92 (s, 3H), 3.60 (q, J=6.4 Hz, 2H), 2.78 (t, J=6.6 Hz, 2H), 2.53 (s, 3H), 1.67 (sext, J=7.0 Hz, 2H), 0.94 (t, J=7.4 Hz, 3H).

### Example 31: N-[3-[(5-tert-butyl-4-methyl-thiazol-2-yl)amino]-3-oxo-propyl]-3-chloro-5-(5-methyl-1,2,4-oxadiazol-3-yl)benzamide

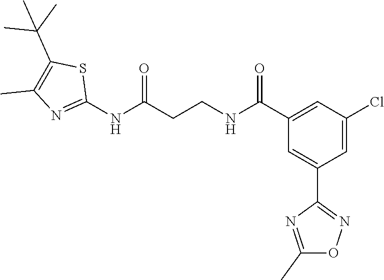 [SMILES: Cc1nc(-c2cc(Cl)cc(C(=O)NCCC(=O)Nc3nc(C)c(C(C)(C)C)s3)c2)no1]

Example 31.1.: Using 3-chloro-5-(5-methyl-1,2,4-oxadiazol-3-yl)benzoic acid [0] in an analogous procedure to 0 afforded 3-[[3-chloro-5-(5-methyl-1,2,4-oxadiazol-3-yl)benzoyl]amino]propanoic acid. HPLC/MS m/z: 310.06, [M+H]⁺, Rt (T): 1.21 min.

Example 31.2.: To a vial was added 3-[[3-chloro-5-(5-methyl-1,2,4-oxadiazol-3-yl)benzoyl]amino]propanoic acid (20.00 mg, 0.0646 mmol), 5-tert-butyl-4-methyl-thiazol-2-ylamine (32.99 mg, 0.1937 mmol), PyBrop (72.25 mg, 0.1550 mmol) followed by dry DMF (0.32 mL) and DIPEA (40.49 uL, 0.2325 mmol). The resulting solution was stirred at rt overnight. The crude was partioned between water (14 mL) and EtOAc (35 mL) and after separation the organic layer was then washed with brine (40 mL), dried (MgSO₄), filtered and concentrated under reduced pressure to dryness. Purification by silica gel column chromatography (eluent: 0-10% MeOH in DCM). Further purified by reverse phase column chromatography (eluent: 30-100% MeOH in water (+0.1% formic acid in both)) to afford 3.97 mg (13%, 0.0086 mmol) of N-[3-[(5-tert-butyl-4-methyl-thiazol-2-yl)amino]-3-oxo-propyl]-3-chloro-5-(5-methyl-1,2,4-oxadiazol-3-yl)benzamide as a colorless solid. HPLC/MS m/z: 462.14 [M+H]⁺, Rt (T): 1.58 min. ¹H NMR (600 MHz, DMSO-d₆) δ 11.85 (s, 1H), 8.95 (t, J=5.5 Hz, 1H), 8.41 (t, J=1.6 Hz, 1H), 8.10 (t, J=1.8 Hz, 1H), 8.08 (t, J=1.8 Hz, 1H), 3.56 (q, J=6.7 Hz, 2H), 2.69 (s, 3H), 2.68 (d, J=7.0 Hz, 2H), 2.30 (s, 3H), 1.35 (s, 9H).

### Example 32: Propyl 4-methyl-2-(3-(3-(5-methyl-1,2,4-oxadiazol-3-yl)-5-(trifluoromethyl)benzamido)propanamido)thiazole-5-carboxylate

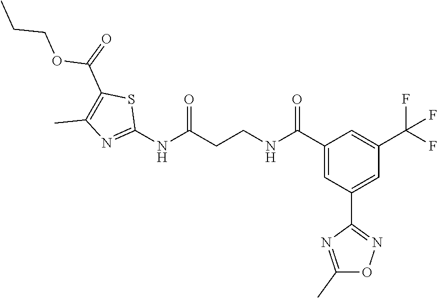 [SMILES: CCCOC(=O)c1sc(NC(=O)CCNC(=O)c2cc(-c3noc(C)n3)cc(C(F)(F)F)c2)nc1C]

Example 32.1.: Propyl 2-[3-[[3-cyano-5-(trifluoromethyl)benzoyl]amino]propanoyl-amino]-4-methyl-thiazole-5-carboxylate [0](105.00 mg, 0.2241 mmol) and TEA (0.03 mL, 0.2241 mmol) were heated at 80° C. in [bmim]OAc (0.22 mL) and hydroxylamine hydrochloride (31.15 mg, 0.4483 mmol) was added. The solution was stirred for 30 min. Solution was diluted in EtOAc (30 mL) and washed with water (3×20 mL), brine (30 mL) before drying over MgSO₄ and concentrating in vacuo to afford 115 mg (102%, 0.2293 mmol) of propyl 2-[3-[[3-(N-hydroxycarbamimidoyl)-5-(trifluoromethyl)benzoyl]amino]propanoylamino]-4-methyl-thiazole-5-carboxylate as a colorless solid which was used in the next step without further purification. HPLC/MS m/z: 502.14, [M+H]⁺, Rt (R): 1.46 min.

Example 32.2.: To a solution of propyl 2-[3-[[3-(N-hydroxycarbamimidoyl)-5-(trifluoromethyl)benzoyl]amino]propanoylamino]-4-methyl-thiazole-5-carboxylate (20.00 mg, 0.0368 mmol) in DMSO (0.40 mL) was added potassium hydroxide (2.06 mg, 0.0368 mmol). The solution was stirred at rt for 1 h. Water (15 mL) was added, and the residue was extracted with EtOAc (3×15 mL). Organic layers were combined, washed with brine (15 mL), dried over MgSO₄ and concentrated in vacuo. Purification by silica gel column chromatography (eluent: 0-55% EtOAc in cyclohexane) follow by further purification by reverse phase column chromatography (eluent: 25-100% MeOH in water (0.1% formic acid as buffer in both)) to afford propyl 4-methyl-2-(3-(3-(5-methyl-1,2,4-oxadiazol-3-yl)-5-(trifluoromethyl)benzamido)propanamido)thiazole-5-carboxylate (6 mg, 31%, 0.0114 mmol) as a colorless solid. HPLC/MS m/z: 527.14, [M+H]⁺, Rt (S): 3.27 min. ¹H NMR (500 MHz, Methanol-d₄) δ 8.74-8.71 (m, 1H), 8.44-8.42 (m, 1H), 8.29-8.27 (m, 1H), 4.20 (t, J=6.6 Hz, 2H), 3.79 (t, J=6.7 Hz, 2H), 2.87 (t, J=6.7 Hz, 2H), 2.68 (s, 3H), 2.56 (d, J=1.2 Hz, 3H), 1.75 (dtd, J=14.0, 7.4, 6.5 Hz, 2H), 1.01 (t, J=7.4 Hz, 3H).

### Example 33: Propyl 2-[3-[(3-methoxycarbonyl-5-nitro-benzoyl)amino]propanoylamino]-4-methyl-thiazole-5-carboxylate

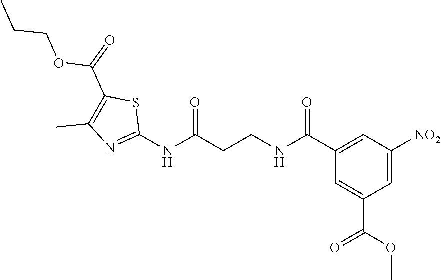 [SMILES: CCCOC(=O)c1sc(NC(=O)CCNC(=O)c2cc(C(=O)OC)cc([N+](=O)[O-])c2)nc1C]

134 mg (58%) light yellow powder (134 mg, 58%, 0.28 mmol). HPLC/MS m/z: 479.12 [M+H]⁺, Rt (R): 1.42 min. ¹H NMR (500 MHz, DMSO-d₆) δ 12.56 (s, 1H), 9.23 (t, J=5.5 Hz, 1H), 8.90 (dd, J=2.2, 1.6 Hz, 1H), 8.79 (t, J=1.6 Hz, 1H), 8.72 (dd, J=2.2, 1.4 Hz, 1H), 4.15 (t, J=6.5 Hz, 2H), 3.95 (s, 3H), 3.63 (q, J=6.4 Hz, 2H), 2.80 (t, J=6.7 Hz, 2H), 2.53 (s, 3H), 1.67 (dtd, J=13.8, 7.4, 6.4 Hz, 2H), 0.94 (t, J=7.4 Hz, 3H).

### Example 34: Ethyl 4-methyl-2-(3-(3-(3-methyl-1,2,4-oxadiazol-5-yl)benzamido)propanamido)thiazole-5-carboxylate

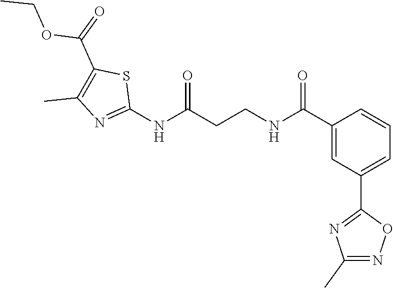 [SMILES: CCOC(=O)c1sc(NC(=O)CCNC(=O)c2cccc(-c3nc(C)no3)c2)nc1C]

Dissolved 3-(3-methyl-1,2,4-oxadiazol-5-yl)benzoic acid (31.74 mg, 0.1555 mmol), ethyl 2-(3-aminopropanoylamino)-4-methyl-thiazole-5-carboxylate (40.00 mg, 0.1555 mmol) and DIPEA (81.45 uL, 0.4664 mmol) in DMF (1.55 mL). Add HATU (54.86 mg, 0.2332 mmol) and stirred at rt for 18 h. The mixture was diluted with EtOAc and washed with water, aqueous saturated bicarbonate, and brine. The organic layer was dried over sodium sulfate and concentrated in vacuo. The residue was purified by silica gel column chromatography (eluent: 0-3% MeOH in DCM). This material was further purified by recrystallisation in warm DMSO to afford ethyl 4-methyl-2-(3-(3-(3-methyl-1,2,4-oxadiazol-5-yl)benzamido)propanamido)thiazole-5-carboxylate (8 mg, 12%, 0.0180 mmol) as colorless needles. HPLC/MS m/z: 444.1 [M+H]⁺, Rt (P): 1.49 min. ¹H NMR (500 MHz, DMSO-d₆) δ 12.54 (s, 1H), 8.93 (t, J=5.5 Hz, 1H), 8.54 (dt, J=1.8, 1.0 Hz, 1H), 8.22 (ddd, J=7.8, 1.8, 1.1 Hz, 1H), 8.13 (ddd, J=7.9, 1.9, 1.2 Hz, 1H), 7.76-7.69 (m, 1H), 4.23 (q, J=7.1 Hz, 2H), 3.61 (q, J=6.5 Hz, 2H), 2.79 (t, J=6.8 Hz, 2H), 2.54 (s, 3H), 2.44 (s, 3H), 1.27 (t, J=7.1 Hz, 3H).

The following examples were prepared by an analogous procedure

### Example 35: Cyclopropylmethyl 4-methyl-2-(3-(3-(5-methyl-1,2,4-oxadiazol-3-yl)benzamido)propanamido)thiazole-5-carboxylate

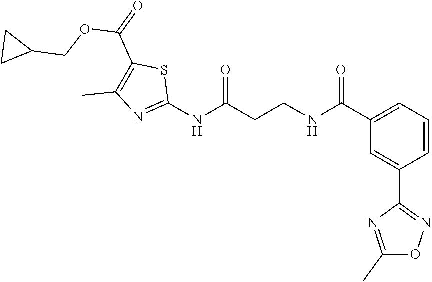 [SMILES: Cc1nc(-c2cccc(C(=O)NCCC(=O)Nc3nc(C)c(C(=O)OCC4CC4)s3)c2)no1]

30 mg (57%) colorless powder. HPLC/MS m/z: 470.15, [M+H]⁺, Rt (S): 2.93 min. ¹H NMR (500 MHz, Methanol-d₄) δ 8.49 (t, J=1.8 Hz, 1H), 8.18 (dt, J=7.7, 1.4 Hz, 1H), 7.96 (dt, J=7.8, 1.5 Hz, 1H), 7.61 (t, J=7.8 Hz, 1H), 4.09 (d, J=7.2 Hz, 2H), 3.77 (t, J=6.7 Hz, 2H), 2.86 (t, J=6.7 Hz, 2H), 2.66 (s, 3H), 2.57 (s, 3H), 1.27-1.19 (m, 1H), 0.65-0.57 (m, 2H), 0.40-0.30 (m, 2H).

### Example 36: Ethyl 2-(3-(3-(5-ethyl-1,2,4-oxadiazol-3-yl)benzamido)propanamido)-4-methylthiazole-5-carboxylate

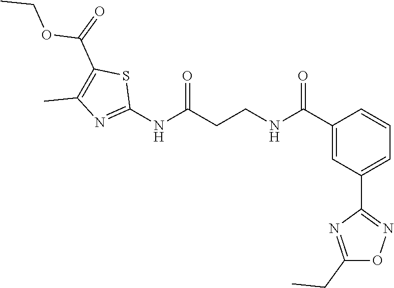 [SMILES: CCOC(=O)c1sc(NC(=O)CCNC(=O)c2cccc(-c3noc(CC)n3)c2)nc1C]

To ethyl 2-(3-aminopropanoylamino)-4-methyl-thiazole-5-carboxylate (40.00 mg, 0.1555 mmol), 3-(5-ethyl-1,2,4-oxadiazol-3-yl)benzoic acid (33.92 mg, 0.1555 mmol) in DMF (1.55 mL) was added DIPEA (81.45 uL, 0.4664 mmol) followed by HATU (54.86 mg, 0.2332 mmol). The reaction was stirred for 18 h at rt. The mixture was diluted with EtOAc and washed with water, aqueous saturated bicarbonate, and brine. The organic layer was dried over sodium sulfate and concentrated in vacuo. The residue was purified by silica gel column chromatography (eluent: 2-8% EtOH in DCM). Further purification by reverse phase column chromatography (eluent: 20-100% MeOH in water (+0.1% formic acid modifier in both)) afforded ethyl-2-(3-(3-(5-ethyl-1,2,4-oxadiazol-3-yl)benzamido)propanamido)-4-methyl-thiazole-5-carboxylate (15 mg, 21%, 0.0328 mmol) as a fine, colorless powder. HPLC/MS m/z: 458.1 [M+H]⁺, Rt (S): 3.00 min. ¹H NMR (500 MHz, DMSO-d₆) δ 12.53 (s, 1H), 8.86 (t, J=5.5 Hz, 1H), 8.45 (t, J=1.7 Hz, 1H), 8.14 (dt, J=7.7, 1.3 Hz, 1H), 8.02 (dt, J=7.9, 1.3 Hz, 1H), 7.65 (t, J=7.8 Hz, 1H), 4.23 (q, J=7.1 Hz, 2H), 3.60 (q, J=6.5 Hz, 2H), 3.03 (q, J=7.6 Hz, 2H), 2.78 (t, J=6.8 Hz, 2H), 2.53 (s, 3H), 1.35 (t, J=7.6 Hz, 3H), 1.27 (t, J=7.1 Hz, 3H).

### Example 37: Ethyl 4-methyl-2-(3-(3-(5-methyl-1,3,4-oxadiazol-2-yl)benzamido)propanamido)thiazole-5-carboxylate

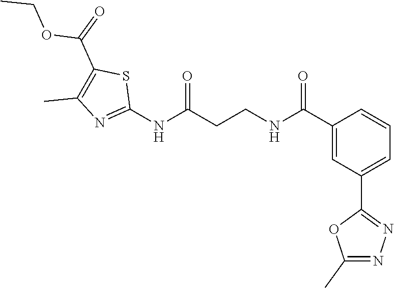 [SMILES: CCOC(=O)c1sc(NC(=O)CCNC(=O)c2cccc(-c3nnc(C)o3)c2)nc1C]

To ethyl 2-(3-aminopropanoylamino)-4-methyl-thiazole-5-carboxylate (40.00 mg, 0.1555 mmol), 3-(5-methyl-1,3,4-oxadiazol-2-yl)benzoic acid (31.74 mg, 0.1555 mmol) in DMF (1.55 mL) was added DIPEA (81.45 uL, 0.4664 mmol) followed by HATU (54.86 mg, 0.2332 mmol). The reaction was stirred overnight at rt and left at rt over weekend. The mixture was diluted with EtOAc (25 mL) and washed with water (25 mL), aqueous saturated bicarbonate (10 mL), The organic layer was dried over sodium sulfate and concentrated in vacuo. Purification by reverse phase column chromatography (eluent: 30-90% MeOH in water (+0.1% formic acid modifier in both)) afforded ethyl-4-methyl-2-(3-(3-(5-methyl-1,3,4-oxadiazol-2-yl)benzamido)propanamido)-thiazole-5-carboxylate (10 mg, 15%, 0.0225 mmol) as a colorless powder. HPLC/MS m/z: 444.1 [M+H]⁺, Rt (S): 2.73 min. ¹H NMR (500 MHz, DMSO-d₆) δ 12.54 (s, 1H), 8.90 (t, J=5.5 Hz, 1H), 8.42 (t, J=1.8 Hz, 1H), 8.14-8.09 (m, 1H), 8.05 (dt, J=7.9, 1.3 Hz, 1H), 7.69 (t, J=7.8 Hz, 1H), 4.23 (q, J=7.1 Hz, 2H), 3.60 (td, J=6.6 Hz, 2H), 2.78 (t, J=6.8 Hz, 2H), 2.60 (s, 3H), 2.53 (s, 3H), 1.27 (t, J=7.1 Hz, 3H).

### Example 38: Ethyl 4-methyl-2-(3-(3-(2-methyl-2H-tetrazol-5-yl)benzamido)propanamido)thiazole-5-carboxylate

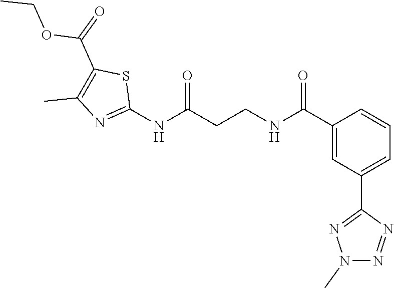 [SMILES: CCOC(=O)c1sc(NC(=O)CCNC(=O)c2cccc(-c3nnn(C)n3)c2)nc1C]

To ethyl 2-(3-aminopropanoylamino)-4-methyl-thiazole-5-carboxylate (40.00 mg, 0.1555 mmol), 3-(2-methyltetrazol-5-yl)benzoic acid (31.74 mg, 0.1555 mmol) in DMF (1.55 mL) was added DIPEA (108.60 uL, 0.6218 mmol) followed by HATU (54.86 mg, 0.2332 mmol). The reaction was stirred overnight at rt. The mixture was diluted with EtOAc and washed with water, aqueous saturated bicarbonate, and brine. The organic layer was dried over magnesium sulfate and concentrated in vacuo. This was purified by reverse phases column chromatography (eluent: 30-100% MeOH in water (+0.1% formic acid modifier in both)) to afford ethyl 4-methyl-2-(3-(3-(2-methyl-2H-tetrazol-5-yl)benzamido)propanamido)thiazole-5-carboxylate (21 mg, 30%, 0.0474 mmol) as a colorless power. HPLC/MS m/z: 444.1 [M+H]⁺, Rt (R): 1.59 min. ¹H NMR (500 MHz, DMSO-d₆) δ 12.54 (s, 1H), 8.86 (t, J=5.5 Hz, 1H), 8.52 (t, J=1.7 Hz, 1H), 8.19 (dt, J=7.7, 1.3 Hz, 1H), 7.98 (dt, J=7.9, 1.3 Hz, 1H), 7.66 (t, J=7.8 Hz, 1H), 4.45 (s, 3H), 4.23 (q, J=7.1 Hz, 2H), 3.60 (q, J=6.5 Hz, 2H), 2.79 (t, J=6.8 Hz, 2H), 2.53 (s, 3H), 1.32-1.24 (m, 3H).

### Example 39: tert-Butyl 4-methyl-2-[3-[[3-(2-methyltetrazol-5-yl)benzoyl]amino]propanoylamino]thiazole-5-carboxylate

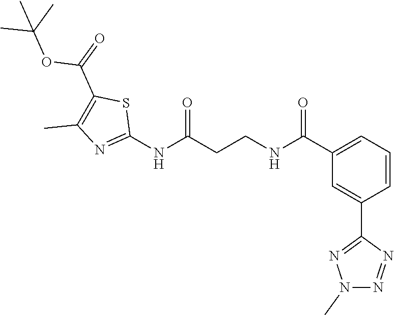 [SMILES: Cc1nc(NC(=O)CCNC(=O)c2cccc(-c3nnn(C)n3)c2)sc1C(=O)OC(C)(C)C]

Example 39.1.: To methyl 3-aminopropanoate hydrochloride (100.00 mg, 0.7164 mmol), 3-(2-Methyl-2H-tetrazol-5-yl)-benzoic acid (146.29 mg, 0.7164 mmol) in DMF (4.21 mL) was added DIPEA (0.50 mL, 2.8657 mmol) followed by HATU (252.83 mg, 1.0747 mmol). The obtained yellow solution was stirred overnight at rt. The reaction mixture was diluted with EtOAc (150 mL) and washed with water (120 mL). The water was extracted with fresh EtOAc (100 mL). The organics were combined and washed with aqueous saturated bicarbonate (150 mL), and brine (200 mL) before drying over MgSO₄. After filtering and concentrating in vacuo methyl 3-[[3-(2-methyltetrazol-5-yl)benzoyl]amino]propanoate (170 mg, 82%, 0.5876 mmol) was obtained as a light-yellow film that was used in the next step without further purification. HPLC/MS m/z: 290.13, [M+H]⁺, Rt (P): 1.15 min.

Example 39.2.: To methyl 3-[[3-(2-methyltetrazol-5-yl)benzoyl]amino]propanoate (170.00 mg, 0.5876 mmol) in THF (2.90 mL) was added water (2.90 mL) followed by LiOH monohydrate (98.63 mg, 2.3506 mmol). After stirring for 45 min LCMS showed complete conversion [product peak m/z=276, rt=1.03 min) so water (25 mL) was added and the THE removed in vacuo. The solution was acidified to pH 3 with 1 M citric acid solution and extracted with EtOAc (2×60 mL). The organics were combined, washed with brine (60 mL) and dried over MgSO₄. This gave 3-[[3-(2-methyltetrazol-5-yl)benzoyl]amino]propanoic acid (137 mg, 85%, 0.4977 mmol) as a colorless solid. HPLC/MS m/z: 276.11, [M+H]⁺, Rt (P): 1.03 min.

Example 39.3.: To a solution of HOBt (29.45 mg, 0.2180 mmol), 3-[[3-(2-methyltetrazol-5-yl)benzoyl]amino]propanoic acid (30.00 mg, 0.1090 mmol) in DMF (0.54 mL) was added EDC (33.84 mg, 0.2180 mmol) and tert-butyl 2-amino-4-methyl-thiazole-5-carboxylate (27.79 mg, 0.1297 mmol). The mixture was stirred for 18 h at 70 0° C. Purified by reverse phase column chromatography (eluent:10-100% MeOH in water (+0.1% formic acid modifier in both) to afford (tert-butyl 4-methyl-2-[3-[[3-(2-methyltetrazol-5-yl)benzoyl]amino]propanoylamino]thiazole-5-carboxylate (41 mg, 80%, 0.0870 mmol) as a colorless solid. HPLC/MS m/z: 472.12 [M+H]⁺, Rt (R): 1.41 min. ¹H NMR (600 MHz, DMSO-d₆) δ 12.46 (s, 1H), 8.86 (t, J=5.5 Hz, 1H), 8.53 (t, J=1.8 Hz, 1H), 8.19 (dt, J=7.7, 1.4 Hz, 1H), 7.99 (dt, J=7.8, 1.5 Hz, 1H), 7.66 (t, J=7.8 Hz, 1H), 4.45 (s, 3H), 3.61 (q, J=6.5 Hz, 2H), 2.79 (t, J=6.8 Hz, 2H), 1.51 (s, 9H).

The following examples were prepared analogously.

### Example 40: Isopropyl 4-methyl-2-[3-[[3-(2-methyltetrazol-5-yl)benzoyl]amino]propanoylamino]thiazole-5-carboxylate

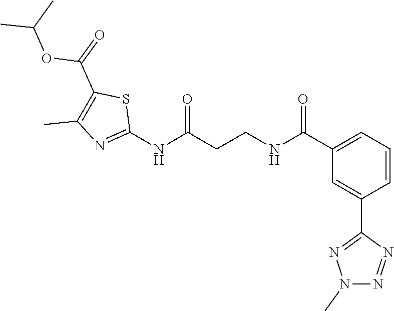 [SMILES: Cc1nc(NC(=O)CCNC(=O)c2cccc(-c3nnn(C)n3)c2)sc1C(=O)OC(C)C]

27 mg (54%) colorless, amorphous solid. HPLC/MS m/z: 458.16 [M+H]⁺, Rt (R): 1.36 min. ¹H NMR (500 MHz, DMSO-d₆) δ 12.51 (s, 1H), 8.86 (t, J=5.5 Hz, 1H), 8.52 (t, J=1.8 Hz, 1H), 8.19 (dt, J=7.7, 1.4 Hz, 1H), 7.98 (dt, J=7.9, 1.4 Hz, 1H), 7.66 (t, J=7.8 Hz, 1H), 5.05 (hept, J=6.3 Hz, 1H), 4.45 (s, 3H), 3.60 (q, J=6.5 Hz, 2H), 2.78 (t, J=6.8 Hz, 2H), 2.53 (s, 3H), 1.28 (d, J=6.2 Hz, 6H).

### Example 41: N-[3-[(5-tert-butyl-4-methyl-thiazol-2-yl)amino]-3-oxo-propyl]-3-(2-methyltetrazol-5-yl)benzamide

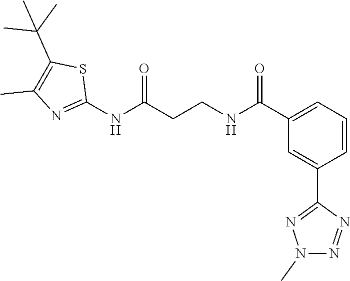 [SMILES: Cc1nc(NC(=O)CCNC(=O)c2cccc(-c3nnn(C)n3)c2)sc1C(C)(C)C]

7 mg (24%) colorless solid. HPLC/MS m/z: 428.19 [M+H]⁺, Rt (T): 1.40 min. ¹H NMR (500 MHz, DMSO-d₆) δ 11.85 (s, 1H), 8.83 (t, J=5.5 Hz, 1H), 8.62-8.47 (m, 1H), 8.27-8.14 (m, 1H), 7.98 (ddd, J=7.9, 1.9, 1.2 Hz, 1H), 7.65 (t, J=7.8 Hz, 1H), 4.45 (s, 3H), 3.57 (q, J=6.6 Hz, 2H), 2.69 (t, J=6.9 Hz, 2H), 2.29 (s, 3H), 1.35 (s, 9H).

### Example 42: Ethyl 2-(3-(3-(ethoxycarbonyl)benzamido)propanamido)-4-methylthiazole-5-carboxylate

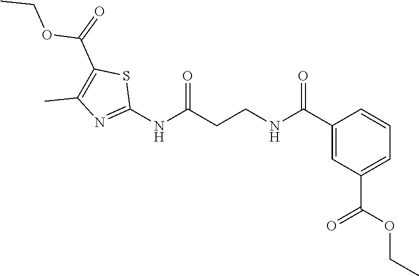 [SMILES: CCOC(=O)c1cccc(C(=O)NCCC(=O)Nc2nc(C)c(C(=O)OCC)s2)c1]

To 3-ethoxycarbonylbenzoic acid (37.73 mg, 0.1943 mmol), ethyl 2-(3-aminopropanoylamino)-4-methyl-thiazole-5-carboxylate (50.00 mg, 0.1943 mmol) in DMF (0.97 mL) was added DIPEA (135.75 uL, 0.7773 mmol) followed by HATU (68.57 mg, 0.2915 mmol). The reaction was stirred overnight at rt. The mixture was diluted with EtOAc and washed with water, aqueous saturated bicarbonate, and brine. The organic layer was dried over magnesium sulfate and concentrated in vacuo. Purified by reverse phase column chromatography (eluent: 30-100% MeOH in water (+0.1% formic acid modifier in both)) followed by silica gel column chromatography (eluent: 20-50% MeCN in DCM) to afford ethyl 2-(3-(3-(ethoxycarbonyl)benzamido)propanamido)-4-methylthiazole-5-carboxylate (5.6 mg, 7%, 0.0129 mmol) as a clear glass. HPLC/MS m/z: 434.1 [M+H]⁺, Rt (S): 2.90 min. ¹H NMR (500 MHz, Acetone-d₆) δ 11.34 (s, 1H), 8.50 (td, J=1.9, 0.6 Hz, 1H), 8.22 (s, 1H), 8.15 (d, J=1.7 Hz, 1H), 8.13 (d, J=1.8 Hz, 1H), 7.61 (td, J=7.8, 0.6 Hz, 1H), 4.37 (q, J=7.1 Hz, 2H), 4.28 (q, J=7.1 Hz, 2H), 3.82 (q, J=6.3 Hz, 2H), 2.97 (t, J=6.5 Hz, 2H), 2.53 (s, 3H), 1.37 (t, J=7.1 Hz, 3H), 1.33 (t, J=7.1 Hz, 3H).

### Example 43: Ethyl 4-methyl-2-(3-(3-methyl-5-(5-methyl-1,2,4-oxadiazol-3-yl)benzamido)propanamido)thiazole-5-carboxylate

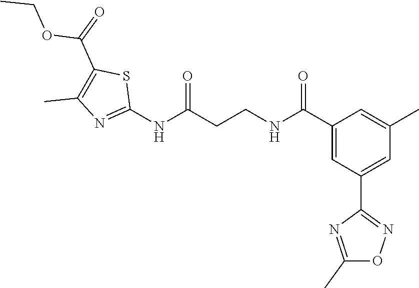 [SMILES: CCOC(=O)c1sc(NC(=O)CCNC(=O)c2cc(C)cc(-c3noc(C)n3)c2)nc1C]

Example 43.1.: Methyl 3-cyano-5-methyl-benzoate (268.00 mg, 1.5299 mmol) was dissolved in MeOH (15.30 mL). Hydroxylamine (50% in water) (0.47 mL, 3.8246 mmol) was added, and the solution was stirred at rt overnight. The solvent was removed in vacuo affording methyl 3-[(Z)-N′-hydroxycarbamimidoyl]-5-methyl-benzoate (159 mg, 50%, 0.7637 mmol) as a brown oil which was used without further purification. HPLC/MS m/z: 194.1 [M+H]⁺, Rt (P): 1.10 min.

Example 43.2.: Acetic acid (0.06 mL, 1.0827 mmol), methyl 3-[(Z)-N′-hydroxycarbamimidoyl]-5-methyl-benzoate (159.00 mg, 0.7637 mmol) and EDC.HCl (206.89 mg, 1.0793 mmol) were dissolved in THF (3.00 mL) and MeCN (3.00 mL) in a microwave vial under nitrogen atmosphere. The reaction mixture was stirred overnight at rt. DIPEA (0.38 mL, 2.1601 mmol) was added and the solution was heated under microwave irradiation for 1 hr at 150 0° C. The solvents were removed in vacuo and DCM (25 mL) and water (25 mL) were added. After shaking the DCM was separated with a Telos phase extractor and the volatiles were removed in vacuo. Purified by reverse phase column chromatography (eluent: 30-100% MeOH in water (+0.1% formic acid modifier in both)) to afford methyl 3-methyl-5-(5-methyl-1,2,4-oxadiazol-3-yl)benzoate (25 mg, 13%, 0.0980 mmol). ¹H NMR (500 MHz, Chloroform-d) δ 8.53 (d, J=1.9 Hz, 1H), 8.07 (td, J=1.7, 0.9 Hz, 1H), 8.00 (td, J=1.7, 0.9 Hz, 1H), 3.94 (s, 3H), 2.67 (s, 3H), 2.47 (d, J=0.8 Hz, 3H).

Example 43.3.: To methyl 3-methyl-5-(5-methyl-1,2,4-oxadiazol-3-yl)benzoate (23.00 mg, 0.0990 mmol) in THF (0.50 mL) was added water (0.50 mL) followed by lithium hydroxide hydrate (8.31 mg, 0.1981 mmol). After stirring overnight, the volatiles were removed in vacuo and the crude partitioned between EtOAc (15 mL) and 1 M HCl (15 mL). The organic was retained and the aqueous extracted with further EtOAc (10 mL). The organics were combined, dried over MgSO₄ and concentrated to give 3-methyl-5-(5-methyl-1,2,4-oxadiazol-3-yl)benzoic acid (17 mg, 59% yield, 75% purity, 0.0584 mmol) as a colorless powder. Used in the next step without further purification. HPLC/MS m/z: 219.1 [M+H]⁺, Rt (R): 1.18 min.

Example 43.4.: To 3-methyl-5-(5-methyl-1,2,4-oxadiazol-3-yl)benzoic acid (17.00 mg, 0.0779 mmol), ethyl 2-(3-aminopropanoylamino)-4-methyl-thiazole-5-carboxylate (20.05 mg, 0.0779 mmol) in DMF (0.73 mL) was added HATU (29.62 mg, 0.0779 mmol) and DIPEA (54.43 uL, 0.3116 mmol). After stirring at rt for 1.5 h the volatiles were removed in vacuo. Purified by silica gel column chromatography (eluent: EtOH gradient in DCM) followed by reverse phase column chromatography (eluent: MeCN gradient in DCM). Final purification by semi-prep HPLC (65%-80% MeOH in water) afforded ethyl 4-methyl-2-(3-(3-methyl-5-(5-methyl-1,2,4-oxadiazol-3-yl)benzamido)propanamido)thiazole-5-carboxylate (4 mg, 11%, 0.0087 mmol) as a clear glass. HPLC/MS m/z: 458.1 [M+H]⁺, Rt (S): 2.90 min. ¹H NMR (500 MHz, Acetone-d₆) δ 11.35 (s, 1H), 8.32 (d, J=1.8 Hz, 1H), 8.23 (s, 1H), 7.98 (s, 1H), 7.89 (s, 1H), 4.28 (q, J=7.1 Hz, 2H), 3.84 (q, J=6.4 Hz, 2H), 2.98 (t, J=6.6 Hz, 2H), 2.67 (s, 3H), 2.52 (s, 3H), 2.46 (s, 3H), 1.33 (t, J=7.1 Hz, 3H).

### Example 44: Propyl 2-(3-(3-(methoxycarbonyl)-5-(5-methyl-1,2,4-oxadiazol-3-yl)benzamido)propanamido)-4-methylthiazole-5-carboxylate

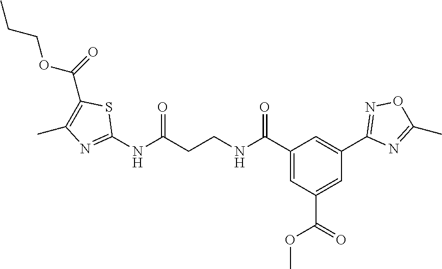 [SMILES: CCCOC(=O)c1sc(NC(=O)CCNC(=O)c2cc(C(=O)OC)cc(-c3noc(C)n3)c2)nc1C]

Example 44.1.: propyl 2-[3-[(3-cyano-5-methoxycarbonyl-benzoyl)amino]propanoyl-amino]-4-methyl-thiazole-5-carboxylate [0](61.00 mg, 0.1330 mmol) and TEA (0.02 mL, 0.1330 mmol) in [bmim]OAc (0.13 mL) were heated at 80° C. and hydroxylamine hydrochloride (18.49 mg, 0.2661 mmol) was added. The solution was stirred for 30 min. The solution was cooled down to rt and water was added (15 mL). Residue was extracted with DCM (3×20 mL). The organic layers were combined, washed with brine (30 mL), dried over MgSO₄ and concentrated under reduced pressure to afford propyl 2-[3-[[3-(N-hydroxycarbamimidoyl)-5-methoxycarbonyl-benzoyl]amino]propanoylamino]-4-methyl-thiazole-5-carboxylate (63.5 mg, 97%, 0.1292 mmol). HPLC/MS m/z: 492.15, [M+H]⁺, Rt (P): 1.35 min.

Example 44.2.: Acetic acid (0.004 mL, 0.0712 mmol), propyl 2-[3-[[3-(N-hydroxycarbamimidoyl)-5-methoxycarbonyl-benzoyl]amino]propanoylamino]-4-methyl-thiazole-5-carboxylate (35.00 mg, 0.0712 mmol) and EDC.HCl (14.33 mg, 0.0748 mmol) were dissolved MeCN (0.43 mL) and THF (0.43 mL) in a 0.5-2 mL microwave vial under nitrogen atmosphere. The solution was stirred overnight at rt. DIPEA (0.03 mL, 0.1709 mmol) was added, and the mixture was heated under microwave for 30 min at 150 0° C. Solvents were removed under vacuum. The residue was dissolved in EtOAc (30 mL), washed with water (30 mL), saturated aqueous bicarbonate (30 mL), brine (30 mL) before drying over magnesium sulfate and concentrating under reduced pressure. Purified by reverse phase column chromatography (eluent: 25-100% MeOH in water (0.1% formic acid as buffer in both)) to afford propyl 2-(3-(3-(methoxycarbonyl)-5-(5-methyl-1,2,4-oxadiazol-3-yl)benzamido)propanamido)-4-methylthiazole-5-carboxylate as a colorless solid. HPLC/MS m/z: 516.15, [M+H]⁺, Rt (S): 3.09 min. ¹H NMR (500 MHz, DMSO-d₆) δ 12.56 (s, 1H), 9.11 (t, J=5.5 Hz, 1H), 8.70 (t, J=1.7 Hz, 1H), 8.63 (t, J=1.7 Hz, 1H), 8.60 (t, J=1.7 Hz, 1H), 4.15 (t, J=6.5 Hz, 2H), 3.93 (s, 3H), 3.62 (q, J=6.4 Hz, 2H), 2.79 (t, J=6.8 Hz, 2H), 2.71 (s, 3H), 2.53 (s, 3H), 1.71-1.63 (m, 2H), 0.94 (t, J=7.4 Hz, 3H).

### Example 45: Ethyl 4-methyl-2-[3-[[7-(5-methyl-1,2,4-oxadiazol-3-yl)-1-isoquinolyl]amino]propanoylamino]thiazole-5-carboxylate

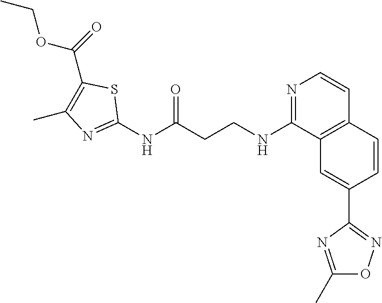 [SMILES: CCOC(=O)c1sc(NC(=O)CCNc2nccc3ccc(-c4noc(C)n4)cc23)nc1C]

Example 45.1.: Nitrogen gas was bubbled through a mixture of 7-bromoisoquinolin-1-ol (0.500 g, 2.23 mmol) and zinc cyanide (0.341 g, 2.90 mmol) in DMF (12.4 mL) for 15 min. Palladium tetrakis(triphenylphosphine) (0.155 g, 0.13 mmol) was added and the mixture heated at 100° C. in a sealed vial for 16 h. The reaction mixture was diluted with brine (100 mL) and extracted with ethyl acetate (100 mL). The organic layer was washed with brine (2×50 mL). Aqueous layers containing some precipitated product were re-extracted with dichloromethane (2×50 mL). Combined organic layers were dried over anhydrous sodium sulfate, filtered, preabsorbed onto silica and purified by column chromatography (eluent: MeOH/DCM=0 to 10% gradient) to afford 0.314 g (83%) of 1-hydroxyisoquinoline-7-carbonitrile. HPLC/MS m/z: 171.05 [M+H]⁺, Rt (R): 0.84 min. ¹H NMR (500 MHz, DMSO-d₆) δ 11.64 (s, 1H), 8.53-8.47 (m, 1H), 8.03 (dd, J=8.3, 1.8 Hz, 1H), 7.84 (d, J=8.3 Hz, 1H), 7.38 (d, J=7.1 Hz, 1H), 6.65 (d, J=7.2 Hz, 1H).

Example 45.2.: 1-Hydroxyisoquinoline-7-carbonitrile (0.215 g, 1.26 mmol) and hydroxylamine hydrochloride (0.176 g, 2.52 mmol) in 1-butyl-3-methylimidazolium acetate (1.3 mL) were heated at 80° C. for 30 min. Water (50 mL) was added and the resulting precipitate filtered, washed with water (50 mL) and dried to afford 0.235 g (92%) of N′,1-dihydroxyisoquinoline-7-carboxamidine. HPLC/MS m/z: 204.07 [M+H]⁺, Rt (R): 0.36 min.

Example 45.3.: N′,1-Dihydroxyisoquinoline-7-carboxamidine (0.235 g, 1.16 mmol) and acetic anhydride (0.13 mL, 1.39 mmol) in acetonitrile (4.6 mL) were heated at 180° C. for 10 min by microwave irradiation. Water (25 mL) was added, and the resulting precipitate filtered, washed with water (25 mL), diethyl ether (2×10 mL) and dried to afford 0.180 g (68%) of 7-(5-methyl-1,2,4-oxadiazol-3-yl)isoquinolin-1-ol. HPLC/MS m/z: 228.08 [M+H]⁺, Rt (R): 1.07 min.

Example 45.4.: 7-(5-methyl-1,2,4-oxadiazol-3-yl)isoquinolin-1-ol (50 mg, 0.22 mmol) in phosphorus oxychloride (2 mL) was heated at 100° C. for 1 h. The mixture was concentrated to afford 0.054 g (100%) of 3-(1-chloro-7-isoquinolyl)-5-methyl-1,2,4-oxadiazole. HPLC/MS m/z: 246/248 Cl split [M+H]⁺, Rt (R): 1.32 min.

Example 45.5.: 3-(1-Chloro-7-isoquinolyl)-5-methyl-1,2,4-oxadiazole (70 mg, 0.28 mmol) and 3-aminopropanenitrile (0.42 mL, 5.69 mmol) in NMP (1.10 mL) was heated at 160° C. for 1 h. Ethyl acetate (30 mL) was added, the resulting precipitate filtered, and the filtrate concentrated. The residue was purified by reverse phase column chromatography (eluent: methanol/water, 0.1% formic acid modifier=20 to 100% gradient) to afford 0.020 g (25%) of 3-[[7-(5-methyl-1,2,4-oxadiazol-3-yl)-1-isoquinolyl]amino]propanenitrile. HPLC/MS m/z: 280.12 [M+H]⁺, Rt (R): 0.79 min.

Example 45.6.: 3-[[7-(5-Methyl-1,2,4-oxadiazol-3-yl)-1-isoquinolyl]amino]-propanenitrile (19 mg, 0.068 mmol) in concentrated aqueous HCl (2 mL) and acetic acid (4 mL) was heated at 80° C. for 1.5 h. The mixture was concentrated to afford 0.020 g (100%) of 3-[[7-(5-methyl-1,2,4-oxadiazol-3-yl)-1-isoquinolyl]amino]-propanoic acid. HPLC/MS m/z: 299.11 [M+H]⁺, Rt (R): 0.93 min.

Example 45.7.: 3-(Ethyliminomethyleneamino)-N,N-dimethyl-propan-1-amine hydrochloride (26 mg, 0.14 mmol) was added to a solution of 3-[[7-(5-methyl-1,2,4-oxadiazol-3-yl)-1-isoquinolyl]amino]propanoic acid (20 mg, 0.068 mmol), ethyl 2-amino-4-methyl-thiazole-5-carboxylate (15 mg, 0.082 mmol) and HOBt (18 mg, 0.014 mmol) in DMF (0.68 mL) under nitrogen. The mixture was heated at 60° C. for 17 h. The solution was cooled, injected directly on to the column and purified by reverse phase column chromatography (eluent: methanol/water, 0.1% formic acid modifier=10 to 100% gradient) to afford 0.012 g (38%) as an off-white solid. HPLC/MS m/z: 467.15 [M+H]⁺, Rt (P): 1.34 min. ¹H NMR (500 MHz, DMSO-d₆) δ 12.56 (s, 1H), 8.87 (s, 1H), 8.15 (dd, J=8.5, 1.6 Hz, 1H), 8.02 (t, J=5.5 Hz, 1H), 7.96 (d, J=5.7 Hz, 1H), 7.85 (d, J=8.5 Hz, 1H), 6.97 (d, J=5.9 Hz, 1H), 4.23 (q, J=7.1 Hz, 2H), 3.81 (q, J=6.5 Hz, 2H), 2.89 (t, J=6.8 Hz, 2H), 2.70 (s, 3H), 2.53 (s, 3H), 1.27 (t, J=7.1 Hz, 3H).

The following examples were prepared analogously:

### Example 46: Methyl 4-methyl-2-[3-[[7-(5-methyl-1,2,4-oxadiazol-3-yl)-1-isoquinolyl]amino]propanoylamino]thiazole-5-carboxylate

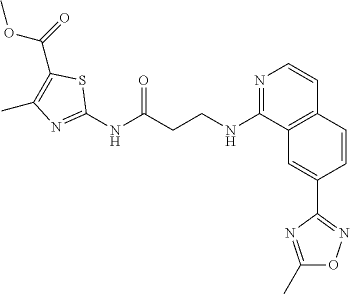 [SMILES: COC(=O)c1sc(NC(=O)CCNc2nccc3ccc(-c4noc(C)n4)cc23)nc1C]

3 mg (6%) colorless, amorphous solid [formate salt]. HPLC/MS m/z: 453.13 [M+H]⁺, Rt (P): 1.29 min. ¹H NMR (500 MHz, MeOD-d₄) δ 8.87 (s, 1H), 8.33-8.15 (m, 2H), 7.91 (d, J=6.1 Hz, 1H), 7.85 (d, J=8.4 Hz, 1H), 7.05 (d, J=6.1 Hz, 1H), 3.96 (t, J=6.6 Hz, 2H), 3.84 (s, 3H), 2.98 (t, J=6.6 Hz, 2H), 2.69 (s, 3H), 2.57 (s, 3H).

### Example 47: N-(5-tert-butyl-4-methyl-thiazol-2-yl)-3-[[6-(5-methyl-1,2,4-oxadiazol-3-yl)quinazolin-4-yl]amino]propanamide formate

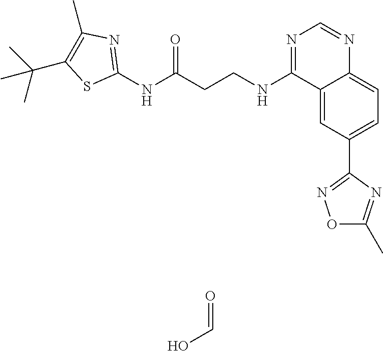 [SMILES: Cc1nc(-c2ccc3ncnc(NCCC(=O)Nc4nc(C)c(C(C)(C)C)s4)c3c2)no1 ; O=CO]

Example 47.1.: 4-Hydroxyquinazoline-6-carbonitrile (500 mg, 2.92 mmol) and triethylamine (0.41 mL, 2.92 mmol) were heated at 80° C. in [bmim]OAc (2.9 mL). Hydroxylamine hydrochloride (406 mg, 5.84 mmol) was added, after which some foaming was observed. The reaction mixture was continued to stir at 80° C. for 30 min. The reaction mixture was cooled down to rt and water was added (50 mL). The precipitate was filtered off, washed with water (50 mL) and dried under reduced pressure to give to yield 536 mg (90%) of N,4-dihydroxyquinazoline-6-carboxamidine. HPLC/MS m/z: 205.1 [M+H]⁺, Rt (U): 0.46 min.

Example 47.2.: A suspension of N,4-dihydroxyquinazoline-6-carboxamidine (480 mg, 2.35 mmol) and acetic anhydride (0.27 mL, 2.82 mmol) in anhydrous ACN (4.70 mL) under an argon atmosphere was heated under microwave irradiation at 180° C. for 10 min. The reaction mixture was cooled to rt and cold water was added (50 mL). The precipitate was filtered off, washed with water (50 mL) and diethyl ether (10 mL), and dried under reduced pressure to yield 293 mg (55%) of 6-(5-methyl-1,2,4-oxadiazol-3-yl)quinazolin-4-ol. HPLC/MS m/z: 229.0727 [M+H]⁺, Rt (U): 1.93 min.

Example 47.3.: A mixture of 6-(5-methyl-1,2,4-oxadiazol-3-yl)quinazolin-4-ol (271 mg, 1.19 mmol) and phosphorus oxychloride (5.0 mL, 53.6 mmol) under an argon atmosphere was heated at 100° C. for 4 h. The solution was cooled to rt and volatiles were removed under reduced pressure. The crude brown solid was used in the next reaction without further purification. HPLC/MS m/z: 243.1 [M+H]⁺, Rt (T): 1.25 min.

Example 47.4.: 3-(4-chloroquinazolin-6-yl)-5-methyl-1,2,4-oxadiazole (150 mg, 0.61 mmol) and sodium 3-aminopropanoate (270 mg, 2.43 mmol) were mixed in anhydrous NMP (2.0 mL) under argon and stirred at rt for 1 h. The reaction mixture was purified by reverse flash chromatography (10-100% MeOH in water) to yield 52 mg (29%) of 3-[[6-(5-methyl-1,2,4-oxadiazol-3-yl)quinazolin-4-yl]amino]propanoic acid as a colorless solid. HPLC/MS m/z: 300.1098 [M+H]⁺, Rt (U): 1.41 min.

Example 47.5.: 3-[[6-(5-methyl-1,2,4-oxadiazol-3-yl)quinazolin-4-yl]amino]propanoic acid (25 mg, 0.084 mmol), 1-(3-dimethylaminopropyl)-3-ethylcarbodiimide hydrochloride (32 mg, 0.167 mmol), 1-hydroxybenzotriazole hydrate (256 mg, 0.167 mmol), and DIPEA (0.04 mL; 0.251 mmol) were mixed in anhydrous DMF (0.17 mL) under an argon atmosphere at rt and stirred for 1 h. 5-tert-butyl-4-methyl-thiazol-2-ylamine (28 mg, 0.167 mmol) was added and the reaction was at 60° C. for 1 h. The mixture was cooled to rt, diluted with EtOAc (30 mL), washed with saturated NaHCO₃ (2×20 mL) and saturated NaCl (20 mL), dried over anhydrous MgSO₄ and concentrated under reduced pressure. The crude material was purified by reverses flash chromatography (10-100% MeOH in water [0.1% FA]). Yield: 24 mg (64%) off-white solid. HPLC/MS m/z: 452.1861 [M+H]⁺, Rt (U): 2.70 min. ¹H NMR (600 MHz, DMSO-d₆) δ 11.86 (s, 1H), 8.93 (d, J=1.9 Hz, 1H), 8.87 (t, J=5.5 Hz, 1H), 8.53 (s, 1H), 8.29 (dd, J=8.7, 1.8 Hz, 1H), 8.15 (s, 1H), 7.81 (d, J=8.7 Hz, 1H), 3.82 (q, J=6.4 Hz, 2H), 2.81 (t, J=6.7 Hz, 2H), 2.70 (s, 3H), 2.28 (s, 3H), 1.34 (s, 9H).

### Example 48.: tert-Butyl 4-methyl-2-((1 s,3s)-3-((6-(5-methyl-1,2,4-oxadiazol-3-yl)quinazolin-4-yl)amino)cyclobutane-1-carboxamido)thiazole-5-carboxylate

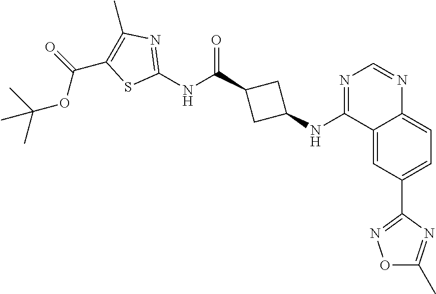 [SMILES: Cc1nc(-c2ccc3ncnc(N[C@H]4C[C@@H](C(=O)Nc5nc(C)c(C(=O)OC(C)(C)C)s5)C4)c3c2)no1]

Example 48.1.: 6-(5-methyl-1,2,4-oxadiazol-3-yl)quinazolin-4-ol (250 mg, 1.10 mmol), cis-methyl 3-aminocyclobutanecarboxylate hydrochloride (200 mg, 1.21 mmol) and DIPEA (0.57 mL, 3.29 mmol) were dissolved in anhydrous DMF (11 mL) at rt under an argon atmosphere. PyBOP (798 mg, 1.53 mmol) was added, and the reaction mixture was stirred for 5 h. The reaction mixture was mixed with water (30 mL) and extracted with EtOAc (3×10 mL). The combined organic layer was washed with saturated NaCl (3×10 mL), dried over anhydrous MgSO₄, filtered and concentrated under reduced pressure. The crude material was purified by flash chromatography (40-100% EtOAc in cyclohexane) to yield 142 mg (38%) of methyl (1 s,3s)-3-((6-(5-methyl-1,2,4-oxadiazol-3-yl)quinazolin-4-yl)amino)cyclobutane-1-carboxylate as an off-white solid. HPLC/MS m/z: 340.1410 [M+H]⁺, Rt (X): 1.87 min.

Example 48.2.: Methyl (1 s,3s)-3-((6-(5-methyl-1,2,4-oxadiazol-3-yl)quinazolin-4-yl)amino)cyclobutane-1-carboxylate (135 mg, 0.40 mmol), THF (1.6 mL), MeOH (0.8 mL) and water (1.6 mL) were mixed at ambient temperature. LiOH monohydrate (33 mg, 0.80 mmol) was added, and the reaction mixture was stirred for 1 h. The reaction mixture was concentrated under reduced pressure and purified by reverse flash chromatography (5-50% MeOH in water [0.1% FA]) to yield 54 mg (42%) of (1s,3s)-3-((6-(5-methyl-1,2,4-oxadiazol-3-yl)quinazolin-4-yl)amino)-cyclobutane-1-carboxylic acid as a colorless powder. HPLC/MS m/z: 326.1247 [M+H]⁺, Rt (X): 1.63 min.

Example 48.3.: (1 s,3s)-3-((6-(5-methyl-1,2,4-oxadiazol-3-yl)quinazolin-4-yl)amino)cyclobutane-1-carboxylic acid (20 mg, 0.062 mmol) and BTFFH (39 mg, 0.123 mmol) were mixed in anhydrous DCM (0.50 mL) in a microwave vial at rt under an argon atmosphere. DIPEA (0.05 mL, 0.277 mmol) was added and the reaction mixture was stirred for 20 min. tert-butyl 2-amino-4-methyl-thiazole-5-carboxylate [0](26 mg, 0.123 mmol) was added and the reaction mixture was heated at 80° C. under microwave irradiation for 1 h. Volatiles were removed under reduced pressure and the crude reaction mixture was directly purified by reverse flash chromatography (10-40% MeOH in water [0.1% FA]). Fractions containing product were filtered through a 1 g SCX-2 cartridge. The compound was released with 2 M NH₃ in MeOH, and solvent was removed under reduced pressure to yield 27 mg (83%) of tert-butyl 4-methyl-2-((1s,3s)-3-((6-(5-methyl-1,2,4-oxadiazol-3-yl)quinazolin-4-yl)amino)cyclobutane-1-carboxamido)thiazole-5-carboxylate as an off-white solid. HPLC/MS m/z: 522.1921 [M+H]⁺, Rt (X): 2.81 min. ¹H NMR (600 MHz, DMSO-d₆) δ 12.43 (s, 1H), 9.06 (d, J=1.8 Hz, 1H), 8.95 (d, J=6.9 Hz, 1H), 8.53 (s, 1H), 8.30 (dd, J=8.7, 1.8 Hz, 1H), 7.81 (d, J=8.7 Hz, 1H), 4.81-4.73 (m, 1H), 3.17-3.10 (m, 1H), 2.71 (s, 3H), 2.64-2.58 (m, 2H), 2.51 (s, 3H), 2.49-2.42 (m, 2H), 1.51 (s, 9H).

### Example 49: tert-Butyl 4-methyl-2-((1 s,3s)-N-methyl-3-((6-(5-methyl-1,2,4-oxadiazol-3-yl)quinazolin-4-yl)amino)cyclobutane-1-carboxamido)thiazole-5-carboxylate formate

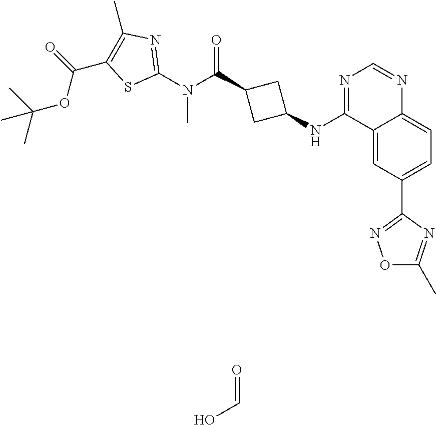 [SMILES: Cc1nc(-c2ccc3ncnc(N[C@H]4C[C@@H](C(=O)N(C)c5nc(C)c(C(=O)OC(C)(C)C)s5)C4)c3c2)no1 ; O=CO]

tert-Butyl 4-methyl-2-((1 s,3s)-3-((6-(5-methyl-1,2,4-oxadiazol-3-yl)quinazolin-4-yl)amino)cyclobutane-1-carboxamido)thiazole-5-carboxylate (20 mg, 0.038 mmol) was dissolved in anhydrous DMF (0.38 mL) under an argon atmosphere and cooled in an ice bath. Sodium hydride 60% dispersion in mineral oil (1.6 mg, 0.040 mmol) was added and the mixture was stirred at 0° C. for 30 min. Iodomethane (2.6 uL, 0.042 mmol) was added. The reaction mixture was allowed to warm to rt and was continued to stir for 5 h. The reaction mixture was purified by reverse flash chromatography (40-80% MeOH in water [0.1% FA]) to yield 13 mg (63%) of tert-butyl 4-methyl-2-((1s,3s)-N-methyl-3-((6-(5-methyl-1,2,4-oxadiazol-3-yl)quinazolin-4-yl)amino)cyclobutane-1-carboxamido)thiazole-5-carboxylate formate as a colorless powder. HPLC/MS m/z: 536.2084 [M+H]⁺, Rt (X): 2.86 min.

### Example 50: N-(5-tert-butyl-4-methyl-thiazol-2-yl)-3-[(7-cyano-1-isoquinolyl)amino]propenamide

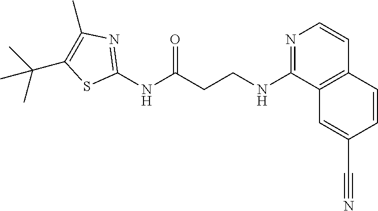 [SMILES: Cc1nc(NC(=O)CCNc2nccc3ccc(C#N)cc23)sc1C(C)(C)C]

Example 50.1.: Isoquinoline-7-carbonitrile (1.00 g, 6.49 mmol) was suspended in anhydrous chloroform (20 mL) under an argon atmosphere and cooled in an ice bath. 3-Chloroperoxybenzoic acid (1.74 g, 7.78 mmol) was added. The reaction mixture was allowed to warm to rt and stirred overnight. The off-white precipitate was filtered off, washed with chloroform and dried under reduced pressure to yield 1.10 g (100%) of 2-oxidoisoquinolin-2-ium-7-carbonitrile as an off-white powder. HPLC/MS m/z: 171.0559 [M+H]⁺, Rt (U): 1.11 min.

Example 50.2.: 2-oxidoisoquinolin-2-ium-7-carbonitrile (300 mg, 1.76 mmol), beta-alanine tert-butyl ester hydrochloride (400 mg, 2.2037 mmol), and DIPEA (1.46 mL, 8.37 mmol) were mixed in anhydrous THF (7.05 mL). PyBroP (1.07 g, 2.29 mmol) was added to the suspension and the mixture was stirred at 60° C. overnight. The reaction mixture was concentrated under reduced pressure and purified by flash chromatography (10-60% EtOAc in cyclohexane) to yield 271 mg (52%) of tert-butyl 3-[(7-cyano-1-isoquinolyl)amino]propanoate as an off-white solid. HPLC/MS m/z: 298.2 [M+H]⁺, Rt (P): 1.15 min.

Example 50.3.: To a solution of tert-butyl 3-[(7-cyano-1-isoquinolyl)amino]-propanoate (271 mg, 0.911 mmol) in DCM (1.82 mL) under an argon atmosphere was added TFA (0.70 mL, 9.11 mmol). The resulting solution was stirred at rt overnight. Volatiles were removed under reduced pressure and the crude was purified by reverse flash chromatography (5-40% MeOH in water [0.1% FA]) to yield 119 mg (54%) of 3-[(7-cyano-1-isoquinolyl)amino]propanoic acid as an off-white solid. HPLC/MS m/z: 242.1 [M+H]⁺, Rt (Q): 0.39 min.

Example 50.4.: Preparation as described for Example 47.5 using 3-[(7-cyano-1-isoquinolyl)amino]propanoic acid (60 mg, 0.249 mmol) and 5-tert-butyl-4-methyl-thiazol-2-ylamine (85 mg, 0.497 mmol). Purification by reverse flash chromatography followed by SCX-2 filtration to afford N-(5-tert-butyl-4-methyl-thiazol-2-yl)-3-[(7-cyano-1-isoquinolyl)amino]propanamide (57 mg, 58%) as an off-white, crystalline solid. HPLC/MS m/z: 394.1698 [M+H]⁺, Rt (U): 2.58 min. ¹H NMR (600 MHz, DMSO-d₆) δ 11.82 (s, 1H), 8.82 (s, 1H), 8.05 (d, J=5.7 Hz, 1H), 7.89 (dd, J=8.5, 1.5 Hz, 1H), 7.87-7.84 (m, 2H), 6.98 (d, J=5.8 Hz, 1H), 3.80-3.75 (m, 2H), 2.78 (t, J=6.8 Hz, 2H), 2.29 (s, 3H), 1.35 (s, 9H).

### Example 51: 3-[(7-cyano-1-isoquinolyl)amino]-N-[4-methyl-5-[1-(trifluoro-methyl)cyclopropyl]thiazol-2-yl]propenamide

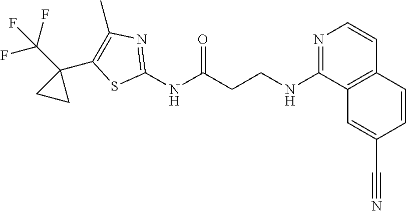 [SMILES: Cc1nc(NC(=O)CCNc2nccc3ccc(C#N)cc23)sc1C1(C(F)(F)F)CC1]

Example 51.1.: 2-Amino-4-methylthiazole (97 mg, 0.850 mmol) and sodium 1-(trifluoromethyl)cyclopropanesulfinate (500 mg, 2.55 mmol) were mixed in diethyl carbonate/water (3:2, 8.50 mL) and cooled in an ice bath. tert-Butylhydroperoxide (70% solution in water, 0.84 mL, 4.25 mmol) was added and the mixture was continued to stir at 0° C. for 5 min before heating at 90° C. for 3 h. The reaction mixture was cooled to rt and mixed with saturated NaHCO₃ (30 mL) and extracted with EtOAc (3×20 mL). The combined organic phase was washed with saturated NaHCO₃ (20 mL) and saturated NaCl (2×20 mL), dried over anhydrous MgSO₄ and concentrated under reduced pressure. The crude reaction mixture was directly purified by reverse flash chromatography (10-80% MeOH in water [0.1% FA]). Fractions containing product were filtered through a 1 g SCX-2 cartridge. The compound was released with 7 M NH₃ in MeOH, and solvent was removed under reduced pressure to yield 65 mg (34%) of 4-methyl-5-[1-(trifluoromethyl)cyclopropyl]thiazol-2-amine as an amorphous, yellow solid. HPLC/MS m/z: 223.0518 [M+H]⁺, Rt (U): 1.65 min.

Example 51.2.: Preparation as described for 0 using 3-[(7-cyano-1-isoquinolyl)amino]propanoic acid (20 mg, 0.083 mmol) and 4-methyl-5-[1-(trifluoromethyl)cyclopropyl]thiazol-2-amine (20 mg, 0.091 mmol). Purification by reverse flash chromatography followed by SCX-2 filtration to afford 3-[(7-cyano-1-isoquinolyl)amino]-N-[4-methyl-5-[1-(trifluoromethyl)cyclopropyl]thiazol-2-yl]propanamide (11.3 mg, 30%) as an off-white solid. HPLC/MS m/z: 446.1252 [M+H]⁺, Rt (U): 2.56 min. ¹H NMR (600 MHz, DMSO-d₆) δ 12.18 (s, 1H), 8.82 (s, 1H), 8.04 (d, J=5.7 Hz, 1H), 7.91-7.84 (m, 3H), 6.98 (d, J=5.7 Hz, 1H), 3.81-3.76 (m, 2H), 2.82 (t, J=6.8 Hz, 2H), 2.26 (s, 3H), 1.47-1.44 (m, 2H), 1.18-1.15 (m, 2H).

### Example 52: 3-[[7-(5-methyl-1,2,4-oxadiazol-3-yl)-1-isoquinolyl]amino]-N-[4-methyl-5-[1-(trifluoromethyl)cyclopropyl]thiazol-2-yl]propenamide

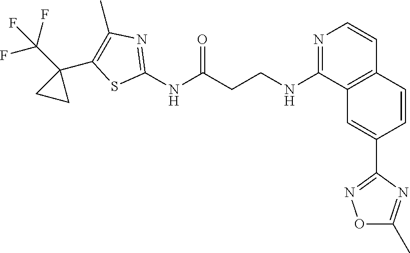 [SMILES: Cc1nc(-c2ccc3ccnc(NCCC(=O)Nc4nc(C)c(C5(C(F)(F)F)CC5)s4)c3c2)no1]

Preparation as described for 0 using 3-[[7-(5-methyl-1,2,4-oxadiazol-3-yl)-1-isoquinolyl]amino]propanoic acid [Example 45.6](25 mg, 0.083 mmol) and 4-methyl-5-[1-(trifluoromethyl)cyclopropyl]thiazol-2-amine [Example 51.1](20 mg, 0.091 mmol). Purification by reverse flash chromatography followed by SCX-2 filtration to afford 3-[[7-(5-methyl-1,2,4-oxadiazol-3-yl)-1-isoquinolyl]amino]-N-[4-methyl-5-[1-(trifluoromethyl)cyclopropyl]thiazol-2-yl]propanamide (13.3 mg, 31%) as an off-white solid. HPLC/MS m/z: 503.1474 [M+H]⁺, Rt (U): 2.70 min. ¹H NMR (600 MHz, DMSO-d₆) δ 12.19 (s, 1H), 8.87 (s, 1H), 8.14 (dd, J=8.5, 1.5 Hz, 1H), 8.00 (t, J=5.5 Hz, 1H), 7.97 (d, J=5.7 Hz, 1H), 7.85 (d, J=8.5 Hz, 1H), 6.97 (d, J=5.7 Hz, 1H), 3.79 (q, J=6.5 Hz, 2H), 2.84 (t, J=6.8 Hz, 2H), 2.70 (s, 3H), 2.25 (s, 3H), 1.47-1.43 (m, 2H), 1.18-1.15 (m, 2H).

### Example 53: N-(5-tert-butyl-4-methyl-thiazol-2-yl)-3-[(7-cyano-5-fluoro-1-isoquinolyl)amino]propenamide

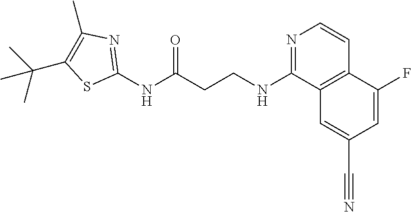 [SMILES: Cc1nc(NC(=O)CCNc2nccc3c(F)cc(C#N)cc23)sc1C(C)(C)C]

Example 53.1.: A mixture of N-Boc-hydroxylamine (9.0 g, 67.6 mmol) and trimethylacetic anhydride (15.1 mL, 74.4 mmol) in anhydrous MeCN (135 mL) under an argon atmosphere was stirred at reflux overnight. Volatiles were removed under reduced pressure and the remaining colourless oil was mixed with EtOAc (100 mL), washed with saturated NaHCO₃ (3×50 mL), dried over anhydrous MgSO₄ and concentrated under reduced pressure to give (tert-butoxycarbonylamino) 2,2-dimethylpropanoate as a colorless, amorphous solid in quantitative yield (14.7 g, 100%) which was used in the next step without further purification. HPLC/MS m/z: 240.1208 [M+Na]⁺, Rt (U): 2.64 min.

Example 53.2.: Trifluoromethanesulfonic acid (5.9 mL, 65.8 mmol) was added to a solution of (tert-butoxycarbonylamino)-2,2-dimethylpropanoate (14.3 g, 65.8 mmol) in diethyl ether (132 mL) at rt under an argon atmosphere and stirred for 3 h. The white, crystalline precipitate was filtered off, washed with diethyl ether and dried under reduced pressure to yield 7.0 g (40%) of 2,2-dimethylpropanoyloxy-ammonium trifluoromethanesulfonate. ¹H NMR (500 MHz, DMSO-d₆) δ 10.68 (s, 3H), 1.22 (s, 9H).

Example 53.3.: 3-Cyano-5-fluorobenzoic acid (1.00 g, 6.06 mmol) and 4-methyl-morpholine (2.00 mL, 18.17 mmol) were mixed in anhydrous THF (12.0 mL) under an argon atmosphere and cooled in an ice bath. Isobutyl chloroformate (0.86 mL, 6.66 mmol) was added and the reaction mixture was continued to stir at 0° C. for 20 min. Then, 2,2-dimethylpropanoyloxyammonium trifluoromethanesulfonate (1.94 g, 7.27 mmol) was added and the reaction mixture was allowed to warm to rt and was continued to stir overnight. The precipitate was filtered off and washed with EtOAc. The filtrate was concentrated under reduced pressure and purified by flash chromatography (0-40% EtOAc in cyclohexane) to yield 1.17 g (73%) of [(3-cyano-5-fluoro-benzoyl)amino]2,2-dimethylpropanoate as a colourless oil. HPLC/MS m/z: 287.0810 [M+Na]⁺, Rt (U): 2.55 min.

Example 53.4.: [(3-cyano-5-fluoro-benzoyl)amino]2,2-dimethylpropanoate (1.00 g, 3.784 mmol), [Cp*RhCl₂]₂(58 mg, 0.095 mmol) and caesium acetate (219 mg, 1.135 mmol) were mixed in MeOH (9.5 mL) at rt under an argon atmosphere. Vinyl acetate (1.05 mL, 11.353 mmol) was added, and the reaction mixture was heated at 45° C. for 18 h. The precipitate was filtered off, washed with water and MeOH, and dried under reduced pressure to yield 428 mg (60%) of 5-fluoro-1-oxo-2H-isoquinoline-7-carbonitrile as an off-white powder. HPLC/MS m/z: 189.1 [M+H]⁺, Rt (T): 0.97 min.

Example 53.5.: A mixture of 5-fluoro-1-hydroxy-isoquinoline-7-carbonitrile (207 mg, 1.10 mmol) and phosphorus oxychloride (4.6 mL, 49.7 mmol) under an argon atmosphere was heated at 110° C. for 4 h. The reaction mixture was cooled to rt and volatiles were removed under reduced pressure to afford the crude 1-chloro-5-fluoro-isoquinoline-7-carbonitrile as an off-white solid in quantitative yield, which was used in the next reaction without further purification. HPLC/MS m/z: 207.0 [M+H]⁺, Rt (U): 1.19 min.

Example 53.6.: 1-Chloro-5-fluoro-isoquinoline-7-carbonitrile (227 mg, 1.10 mmol) and sodium 3-aminopropanoate (488 mg, 4.40 mmol) were mixed in anhydrous NMP (3.7 mL) under an argon atmosphere and stirred at 60° C. overnight. The reaction mixture was purified by reverse flash chromatography (5-30% MeOH in water [0.1% FA]) to yield 107 mg (38%) of 3-[(7-cyano-5-fluoro-1-isoquinolyl)-amino]propanoic acid as an off-white solid. HPLC/MS m/z: 260.0840 [M+H]⁺, Rt (U): 1.33 min.

Example 53.7.: Preparation as described for Example 47.5 using 3-[(7-cyano-5-fluoro-1-isoquinolyl)amino]propanoic acid (50 mg, 0.193 mmol) and 5-tert-butyl-4-methyl-thiazol-2-ylamine (39 mg, 0.231 mmol). Purification by reverse flash chromatography followed by SCX-2 filtration to afford N-(5-tert-butyl-4-methyl-thiazol-2-yl)-3-[(7-cyano-5-fluoro-1-isoquinolyl)amino]propanamide (61 mg, 77%) as an off-white, crystalline solid. HPLC/MS m/z: 412.1602 [M+H]⁺, Rt (Q): 2.93 min. ¹H NMR (500 MHz, DMSO-d₆) δ 11.84 (s, 1H), 8.70 (d, J=1.2 Hz, 1H), 8.15 (d, J=5.8 Hz, 1H), 8.02 (t, J=5.4 Hz, 1H), 7.93 (dd, J=10.1, 1.3 Hz, 1H), 7.00 (dd, J=5.8, 0.9 Hz, 1H), 3.81-3.75 (m, 2H), 2.78 (t, J=6.8 Hz, 2H), 2.29 (s, 3H), 1.35 (s, 9H).

### Example 54: Propyl 4-methyl-2-(3-((7-(5-methyl-1,2,4-oxadiazol-3-yl)isoquinolin-1-yl)amino)propanamido)thiazole-5-carboxylate

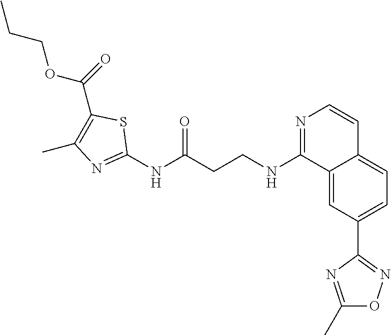 [SMILES: CCCOC(=O)c1sc(NC(=O)CCNc2nccc3ccc(-c4noc(C)n4)cc23)nc1C]

Example 54.1.: To sodium 3-aminopropanoate (213.42 mg, 1.9214 mmol) was added NMP (1.60 mL). The vial was heated to 100° C. and stirred for 30 min before 3-(1-chloro-7-isoquinolyl)-5-methyl-1,2,4-oxadiazole [Example 45](118.00 mg, 0.4803 mmol) was added under a nitrogen atmosphere. Stirred at 100° C. overnight. Reaction mixture cooled and water (3 mL) added. Purified by reverse phase column chromatography (eluent: 20-70% MeOH in water (+0.1% formic acid modifier in both) to afford 3-[[7-(5-methyl-1,2,4-oxadiazol-3-yl)-1-isoquinolyl]amino]propanoic acid (123 mg, 86%, 0.4123 mmol) as a colorless powder. HPLC/MS m/z: 299.1, [M+H]⁺, Rt (P): 0.98 min.

Example 54.2.: To a mixture of 3-[[7-(5-methyl-1,2,4-oxadiazol-3-yl)-1-isoquinolyl]amino]propanoic acid (30.00 mg, 0.1006 mmol), EDC.HCl (38.56 mg, 0.2011 mmol), HOBt (27.18 mg, 0.2011 mmol) and propyl 2-amino-4-methyl-thiazole-5-carboxylate [Example 63.1](30.21 mg, 0.1509 mmol) was added under nitrogen atmosphere DMF (0.40 mL). The resulting solution was stirred at 70° C. overnight. LCMS showed total conversion. Purified by reverse phase column chromatography (eluent: 10-100% MeOH in water (+0.1% formic acid)) followed by ion exchange SCX-II column chromatography, washing with MeOH before eluting with 2M NH₃ in MeOH to afford propyl 4-methyl-2-(3-((7-(5-methyl-1,2,4-oxadiazol-3-yl)isoquinolin-1-yl)amino)propanamido)thiazole-5-carboxylate (32 mg, 66%, 0.0666 mmol) as a colorless solid. HPLC/MS m/z: 481.2, [M+H]⁺, Rt (Q): 2.65 min. ¹H NMR (600 MHz, DMSO-d₆) δ 12.56 (s, 1H), 8.88 (d, J=1.5 Hz, 1H), 8.15 (dd, J=8.5, 1.5 Hz, 1H), 8.02 (t, J=5.4 Hz, 1H), 7.97 (d, J=5.7 Hz, 1H), 7.86 (d, J=8.5 Hz, 1H), 6.98 (d, J=5.7 Hz, 1H), 4.16 (t, J=6.5 Hz, 2H), 3.82 (q, J=6.4 Hz, 2H), 2.90 (t, J=6.8 Hz, 2H), 2.70 (s, 3H), 2.54 (s, 3H), 1.68 (sext, J=7.1 Hz, 2H), 0.95 (t, J=7.4 Hz, 3H).

The following examples were prepared by an analogous procedure:

### Example 55: N-(5-cyanothiazol-2-yl)-3-((7-(5-methyl-1,2,4-oxadiazol-3-yl)isoquinolin-1-yl)amino)propenamide

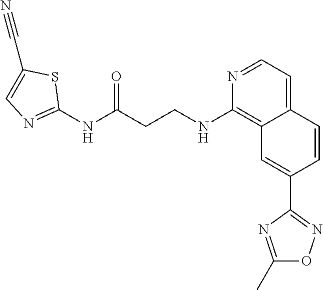 [SMILES: Cc1nc(-c2ccc3ccnc(NCCC(=O)Nc4ncc(C#N)s4)c3c2)no1]

38 mg (54%) colorless solid. HPLC/MS m/z: 406.11, [M+H]⁺, Rt (U): 2.11 min. ¹H NMR (600 MHz, DMSO-d₆) δ 13.00 (s, 1H), 8.87 (s, 1H), 8.35 (s, 1H), 8.15 (dd, J=8.5, 1.5 Hz, 1H), 8.05 (t, J=5.5 Hz, 1H), 7.95 (d, J=5.7 Hz, 1H), 7.85 (d, J=8.5 Hz, 1H), 6.97 (d, J=5.7 Hz, 1H), 3.83 (q, J=6.3 Hz, 2H), 2.94 (t, J=6.7 Hz, 2H), 2.70 (s, 3H).

### Example 56: 3-((7-(5-methyl-1,2,4-oxadiazol-3-yl)isoquinolin-1-yl)amino)-N-(1-methyl-5-(trifluoromethyl)-1 H-pyrazol-3-yl)propenamide

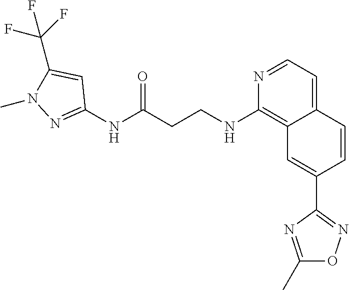 [SMILES: Cc1nc(-c2ccc3ccnc(NCCC(=O)Nc4cc(C(F)(F)F)n(C)n4)c3c2)no1]

14 mg (47%) colorless solid. HPLC/MS m/z: 446.16, [M+H]⁺, Rt (U): 2.39 min. ¹H NMR (600 MHz, DMSO-d₆) δ 10.77 (s, 1H), 8.88 (s, 1H), 8.14 (dd, J=8.5, 1.5 Hz, 1H), 7.98 (d, J=5.7 Hz, 1H), 7.96 (t, J=6.5 Hz, 1H), 7.85 (d, J=8.5 Hz, 1H), 7.00 (s, 1H), 6.96 (d, J=5.7 Hz, 1H), 3.85 (s, 3H), 3.77 (q, J=6.8 Hz, 2H), 2.75 (t, J=7.0 Hz, 2H), 2.70 (s, 3H).

### Example 57: 3-((7-(5-methyl-1,2,4-oxadiazol-3-yl)isoquinolin-1-yl)amino)-N-(1-methyl-5-pentyl-1 H-pyrazol-3-yl)propanamide

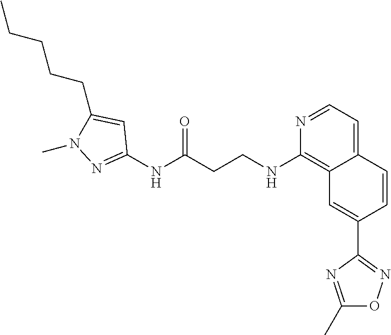 [SMILES: CCCCCc1cc(NC(=O)CCNc2nccc3ccc(-c4noc(C)n4)cc23)nn1C]

Example 57.1.: To a solution of diisopropylamine (0.33 mL, 2.3603 mmol) in THE (1.9 mL) was added 1.6 M nBuLi (1.48 mL, 2.3603 mmol) at −78 0° C. The solution was stirred for 15 min at 0 C. Then, the solution was rechilled at −78° C. and a solution of 1-methyl-3-nitro-1 H-pyrazole (250.00 mg, 1.967 mmol) in THF (1.9 mL) was added. After stirring at this temperature for 1 h, a solution of iodine (599.07 mg, 2.3603 mmol) in THF (0.8 mL) was added and the reaction mixture was allowed to warm to rt and stirred for 2 h. The reaction was quenched with saturated solution of Na₂S₂O₃ (10 mL) and NH₄Cl (25 mL). Residue was extracted with EtOAc (2×15 mL). Organic layer was combined, dried over magnesium sulfate and concentrated under vacuum. Purification by silica gel column chromatography (eluent: 0-40% EtOAc in cyclohexane) to afford 5-iodo-1-methyl-3-nitro-pyrazole (240 mg, 48%, 0.9486 mmol) as a light brown solid. ¹H NMR (600 MHz, Chloroform-d) δ 7.09 (s, 1H), 4.06 (s, 3H).

Example 57.2.: To a mixture of 5-iodo-1-methyl-3-nitro-pyrazole (240.00 mg, 0.9486 mmol), copper(I) iodide (10.84 mg, 0.0569 mmol), bis(triphenylphosphine)-palladium(II) chloride (39.95 mg, 0.0569 mmol) and 1-pentyne (0.14 mL, 1.4229 mmol) was added a degassed solution of DMF (1.50 mL)/TEA (3.00 mL). The resulting solution was heated under microwave irradiation for 30 min at 90° C. Volatiles were removed under reduced pressure. Purification by silica gel column chromatography (eluent: 0-8% EtOAc in cyclohexane) afforded 1-methyl-3-nitro-5-pent-1-ynyl-pyrazole (120 mg, 65%, 0.6211 mmol) as a brown oil. HPLC/MS m/z: 194.09, [M+H]⁺, Rt (P): 1.43 min.

Example 57.3.: To a solution of 1-methyl-3-nitro-5-pent-1-ynyl-pyrazole (60.00 mg, 0.3106 mmol) in EtOH (1.55 mL) was added 10% Pd/C (33.05 mg, 0.0311 mmol). The flask was placed under H₂ atmosphere and the solution was stirred at rt for 7.5 h. Pd/C was filtered through a pad of celite and washed with MeOH (5 mL). Purification by ion exchange SCX-II column chromatography, washing with MeOH before eluting with 2M NH₃ in MeOH to give 1-methyl-5-pentyl-pyrazol-3-amine (41 mg, 79%, 0.2451 mmol) as a yellow oil which was used in the next step without further purification. HPLC/MS m/z: 168.15, [M+H]⁺, Rt (T): 0.95 min.

Example 57.4.: An analogous procedure to Example 54.2 using 1-methyl-5-pentyl-pyrazol-3-amine and 3-[[7-(5-methyl-1,2,4-oxadiazol-3-yl)-1-isoquinolyl]amino]-propanoic acid afforded 3-((7-(5-methyl-1,2,4-oxadiazol-3-yl)isoquinolin-1-yl)amino)-N-(1-methyl-5-pentyl-1 H-pyrazol-3-yl)propanamide (10.2 mg, 34%, 0.0228 mmol) as a colorless solid. HPLC/MS m/z: 448.25, [M+H]⁺, Rt (U): 2.62 min. ¹H NMR (600 MHz, DMSO-d₆) δ 10.29 (s, 1H), 8.88 (s, 1H), 8.14 (dd, J=8.5, 1.6 Hz, 1H), 7.98 (d, J=5.7 Hz, 1H), 7.93 (t, J=5.4 Hz, 1H), 7.84 (d, J=8.5 Hz, 1H), 6.95 (d, J=5.7 Hz, 1H), 6.30 (s, 1H), 3.74 (td, J=7.1, 5.2 Hz, 2H), 3.59 (s, 3H), 2.69 (d, J=8.1 Hz, 5H), 2.54 (t, J=7.6 Hz, 2H), 1.55 (quint, J=7.3 Hz, 2H), 1.37-1.26 (m, 4H), 0.91-0.83 (m, 3H).

### Example 58: N-(5-(tert-butyl)-4-methylthiazol-2-yl)-3-((7-(5-methyl-2H-tetrazol-2-yl)isoquinolin-1-yl)amino)propenamide

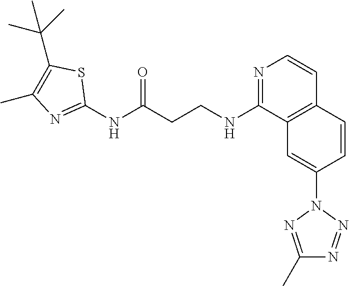 [SMILES: Cc1nnn(-c2ccc3ccnc(NCCC(=O)Nc4nc(C)c(C(C)(C)C)s4)c3c2)n1]

Example 58.1.: 7-Aminoisoquinoline (720.85 mg, 5 mmol) was dissolved in a mixture of 50% aqueous hydrofluroboric acid (1.25 mL, 19.929 mmol) and EtOH (1.50 mL). The reaction mixture was cooled to 0° C. and tert-butyl nitrite (1.35 mL, 10 mmol) was added dropwise. Stirred for 1 h at rt, diethyl ether (10 mL) was added to precipitate the diazonium compound that was filtered off and washed with diethyl ether (3×5 mL) and dried to afford isoquinoline-7-diazonium tetrafluoroborate (1.02 g, 84%, 4.1857 mmol) as a dark red powder. ¹H NMR (500 MHz, DMSO-d₆) δ 9.77 (d, J=0.9 Hz, 1H), 9.75 (d, J=2.1 Hz, 1H), 8.97 (d, J=5.7 Hz, 1H), 8.65 (dd, J=9.0, 2.1 Hz, 1H), 8.50 (d, J=9.1 Hz, 1H), 8.22-8.17 (m, 1H).

Example 58.2.: To a solution of acetamidine hydrochloride (155.64 mg, 1.6463 mmol) and potassium carbonate (1.14 g, 8.2315 mmol) in DMSO (8.00 mL) was added isoquinoline-7-diazonium tetrafluoroborate (400.00 mg, 1.6463 mmol) in portions. After stirring for 1.5 h at rt, potassium iodide (409.93 mg, 2.4694 mmol) and iodine (501.41 mg, 1.9756 mmol) were added. Stirred at rt for 1.5 h before a solution of brine and sodium thiosulfate were added to the mixture. The product was extracted with EtOAc. The organic layer was dried over MgSO₄ and concentrated in vacuo. Purification by silica gel column chromatography (eluent: 30-60% EtOAc in cyclohexane) afforded 7-(5-methyltetrazol-2-yl)isoquinoline (62 mg, 18%, 0.2935 mmol) as a yellow powder. HPLC/MS m/z: 212.1, [M+H]⁺, Rt (T): 0.96 min.

Example 58.3.: 7-(5-methyltetrazol-2-yl)isoquinoline (120.00 mg, 0.5681 mmol) was dissolved in CHCl₃ (1.54 mL) and cooled in an ice bath. 3-Chloroperoxybenzoic acid (0.15 g, 0.6818 mmol) was added. The reaction was stirred at rt for 2 h. K₂CO₃ (0.31 g, 2.2725 mmol) was added, stirred for 0.5 h before filtering. The filtrate was concentrated in vacuo. Residue dissolved in CHCl₃, washed a saturated solution of NaHCO₃, dried over MgSO₄ and concentrated in vacuo to give 7-(5-methyltetrazol-2-yl)-2-oxido-isoquinolin-2-ium (122 mg, 95%, 0.5369 mmol) as a yellow powder. HPLC/MS m/z: 228.1, [M+H]⁺, Rt (P): 1.04 min.

Example 58.4.: 7-(5-methyltetrazol-2-yl)-2-oxido-isoquinolin-2-ium (120.00 mg, 0.5281 mmol) and beta-alanine tert-butyl ester hydrochloride (124.72 mg, 0.6866 mmol) were dissolved in dry DCM (2.80 mL). DIPEA (0.43 mL, 2.4822 mmol) was added followed by PyBroP (369.30 mg, 0.7922 mmol) and the flask stirred at rt overnight. 1 equivalent of PyBrop, amine and DIPEA were added, the mixture was stirred at 30° C. for 20 h. The solvent was removed in vacuo. Purification by silica gel column chromatography (eluent: 5-40%: EtOAc in cyclohexane) afforded tert-butyl 3-[[7-(5-methyltetrazol-2-yl)-1-isoquinolyl]amino]propanoate (95 mg, 51%, 0.2681 mmol) as an orange powder. HPLC/MS m/z: 355.2, [M+H]⁺, Rt (T): 1.13 min.

Example 58.5.: tert-Butyl 3-[[7-(5-methyltetrazol-2-yl)-1-isoquinolyl]amino]-propanoate (85.00 mg, 0.2398 mmol) and potassium hydroxide (134.57 mg, 2.3984 mmol) were dissolved in THF (1.20 mL). Few drops of water and MeOH were added. The reaction was stirred at 50° C. for 2 h. Further potassium hydroxide (134.57 mg, 2.3984 mmol) and more water were added, stirred at 50° C. for 3 h. Purified by reverse phase column chromatography (eluent: 20-80% MeOH/H₂O+0.1% formic acid) to afford 3-[[7-(5-methyltetrazol-2-yl)-1-isoquinolyl]amino]-propanoic acid (60 mg, 84%, 0.2011 mmol) as a colorless powder. HPLC/MS m/z: 299.1, [M+H]⁺, Rt (P): 0.98 min.

Example 58.6.: 3-[[7-(5-methyltetrazol-2-yl)-1-isoquinolyl]amino]propanoic acid (60.00 mg, 0.2011 mmol), EDC (116.84 mg, 0.6034 mmol), HOBt (92.41 mg, 0.6034 mmol) and 5-tert-butyl-4-methyl-thiazol-2-ylamine (102.75 mg, 0.6034 mmol) were dissolved in dry DMF (2.01 mL). DIPEA (176.05 uL, 1.0057 mmol) was added and the reaction mixture was stirred at 50° C. for 21 h. Purified by reverse phase column chromatography (eluent: 30-100% MeOH/H₂O+0.1% formic acid) followed by ion exchange SCX-II column chromatography, washing with MeOH before eluting with 2M NH₃ in MeOH. Trituration with MeOH afforded N-(5-(tert-butyl)-4-methylthiazol-2-yl)-3-((7-(5-methyl-2H-tetrazol-2-yl)isoquinolin-1-yl)amino)propanamide (8 mg, 9%, 0.0172 mmol) as a colorless powder. HPLC/MS m/z: 451.2, [M+H]⁺, Rt (Q): 2.69 min. ¹H NMR (600 MHz, DMSO-d₆) δ 11.84 (s, 1H), 8.96 (d, J=2.1 Hz, 1H), 8.28 (dd, J=8.8, 2.1 Hz, 1H), 8.06-7.90 (m, 3H), 7.02 (d, J=5.8 Hz, 1H), 3.81-3.74 (m, 2H), 2.80 (t, J=6.8 Hz, 2H), 2.62 (s, 3H), 2.28 (s, 3H), 1.35 (s, 9H).

### Example 59: N-(3-((5-(tert-butyl)-4-methylthiazol-2-yl)amino)-3-oxopropyl)-3-(5-methyl-2H-tetrazol-2-yl)benzamide

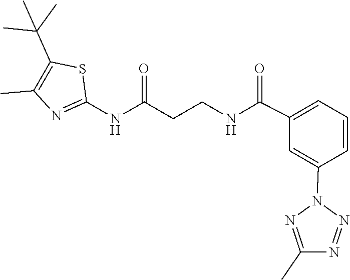 [SMILES: Cc1nnn(-c2cccc(C(=O)NCCC(=O)Nc3nc(C)c(C(C)(C)C)s3)c2)n1]

Example 59.1.: Ethyl 3-amino benzoate (0.81 mL, 5 mmol) was dissolved in a mixture of water (1.00 mL) and 50% aqueous hydrofluroboric acid (1.90 mL, 30.293 mmol). Sodium nitrite (689.90 mg, 10 mmol), dissolved in water (1.00 mL), was added dropwise. The reaction was stirred at 0° C. for 30 min. The reaction was filtered and the solid was washed with Et₂O and dried to afford 3-(ethoxycarbonyl)-benzenediazonium tetrafluoroborate (1.19 g, 90%, 4.489 mmol). ¹H NMR (600 MHz, DMSO-d₆) δ 9.26 (t, J=1.9 Hz, 1H), 8.88 (ddd, J=8.3, 2.1, 1.1 Hz, 1H), 8.70 (dt, J=8.0, 1.4 Hz, 1H), 8.12 (t, J=8.1 Hz, 1H), 4.42 (q, J=7.1 Hz, 2H), 1.37 (t, J=7.1 Hz, 3H).

Example 59.2.: To a solution of acetamidine hydrochloride (0.42 g, 4.47 mmol) and potassium carbonate (3.09 g, 22.35 mmol) in DMSO (21.72 mL) was added 3-(ethoxycarbonyl)benzenediazonium tetrafluoroborate (1.18 g, 4.47 mmol) in portions. After stirring for 1.5 h at rt, potassium iodide (1113.04 mg, 6.7051 mmol) and iodine (1361.45 mg, 5.364 mmol) were added. Stirred at rt for 1.5 h before a solution of brine and sodium thiosulfate were added to the mixture. The product was extrated with EtOAc, the organic layer was dried over MgSO₄, and concentrated in vacuo. Purification by silica gel column chromatography (eluent: 0-30% EtOAc in cyclohexane) to give ethyl 3-(5-methyltetrazol-2-yl)benzoate (250 mg, 24%, 1.0765 mmol) as an orange powder. HPLC/MS m/z: 233.1, [M+H]⁺, Rt (P): 1.46 min.

Example 59.3.: Ethyl 3-(5-methyltetrazol-2-yl)benzoate (240.00 mg, 1.0334 mmol) was dissolved in THF (5.17 mL). LiOH (247.50 mg, 10.334 mmol) was added, and the mixture was stirred at 50° C. for 1 h. Water (1 mL) and methanol (1 mL) was added, stirred for 2 h at 50 0° C. Cooled to rt, diluted with EtOAc and water and the pH was adjusted to pH ˜2-3. The product was extracted with EtOAc, dried over MgSO₄ and concentrated in vacuo to give 3-(5-methyltetrazol-2-yl)benzoic acid (222 mg, 105%, 1.0872 mmol) as a yellow powder. HPLC/MS m/z: 205.1, [M+H]⁺, Rt (P): 1.23 min.

Example 59.4.: 3-[[3-(5-methyltetrazol-2-yl)benzoyl]amino]propanoic acid (185.00 mg, 0.6721 mmol), EDC (260.28 mg, 1.3442 mmol), HOBt (205.85 mg, 1.3442 mmol) and 5-tert-butyl-4-methyl-thiazol-2-ylamine (114.44 mg, 0.6721 mmol) were dissolved in dry DMF (3.36 mL). DIPEA (235.30 uL, 1.3442 mmol) was added and the reaction mixture was stirred at 50° C. for 2 h. The mixture was cooled to rt and diluted with a mixture of water and EtOAc. The product was extracted with EtOAc, washed with water, dried over MgSO₄ and concentrated in vacuo. Purified by reverse phase column chromatography (eluent: 30-100% MeOH/H₂O+0.1% formic acid) to afford N-(3-((5-(tert-butyl)-4-methylthiazol-2-yl)amino)-3-oxopropyl)-3-(5-methyl-2H-tetrazol-2-yl)benzamide (60 mg, 20%, 0.1361 mmol) as a colorless powder. HPLC/MS m/z: 428.2, [M+H]⁺, Rt (Q): 2.98 min. ¹H NMR (500 MHz, DMSO-d₆) δ 11.86 (s, 1H), 8.92 (t, J=5.6 Hz, 1H), 8.52 (t, J=1.9 Hz, 1H), 8.21 (ddd, J=8.1, 2.2, 1.0 Hz, 1H), 8.03 (ddd, J=7.8, 1.6, 1.0 Hz, 1H), 7.75 (t, J=8.0 Hz, 1H), 3.58 (q, J=6.7 Hz, 2H), 2.69 (t, J=6.9 Hz, 2H), 2.61 (s, 3H), 2.29 (s, 3H), 1.35 (s, 9H).

### Example 60: (S)-N-(1-((5-(tert-butyl)-4-methylthiazol-2-yl)amino)-6-(methylamino)-1-oxohexan-3-yl)-3-(5-methyl-2H-tetrazol-2-yl)benzamide

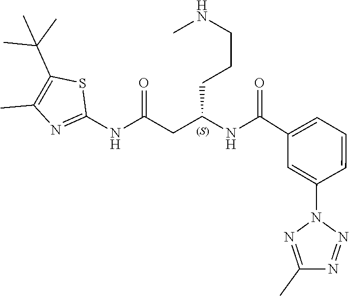

Example 60.1.: Ethyl 3-amino benzoate (0.81 mL, 5 mmol) was dissolved in a mixture of water (1.00 mL) and 50% aqueous hydrofluroboric acid (1.90 mL, 30.293 mmol). Sodiumnitrite (689.90 mg, 10 mmol), dissolved in water (1.00 mL), was added dropwise. The reaction was stirred at 0° C. for 30 min. The reaction was filtered and the solid was washed with Et₂O. The solid was transferred into a flask and dried in vacuo for 30 min to give 3-ethoxycarbonylbenzenediazonium; tetrafluoroboron (1.1 g, 83%, 4.167 mmol) as a pale pink powder. HPLC/MS m/z: 177.07, [M+H]⁺, Rt (T): 0.31 min.

Example 60.2.: To a solution of acetamidine hydrochloride (0.39 g, 4.167 mmol) and potassium carbonate (2.88 g, 20.835 mmol) in DMSO (20.25 mL) was added 3-ethoxycarbonylbenzenediazonium; tetrafluoroboron (1.10 g, 4.167 mmol) in portions. After stirring for 1.5 h at rt potassium iodide (1.04 g, 6.2505 mmol) and iodine (1.27 g, 5.0004 mmol) were added, stirred at rt for 1.5 h. A solution of brine and sodium thiosulfate were added to the mixture. The product was extrated with EtOAc, the organic layer was dried over MgSO₄, and concentrated in vacuo. Purification by silica gel column chromatography (eluent: 5-30% EtOAc in cyclohexane) to afford ethyl 3-(5-methyltetrazol-2-yl)benzoate (582 mg, 60%, 2.506 mmol) as yellow powder. HPLC/MS m/z: 233.10, [M+H]⁺, Rt (T): 1.36 min.

Example 60.3.: Ethyl 3-(5-methyltetrazol-2-yl)benzoate (300.00 mg, 1.2918 mmol) was dissolved in THF (6.46 mL). LiOH (309.38 mg, 12.918 mmol) was added, and the mixture was stirred at 50° C. for 1 h. 1 mL of water and methanol was added to help to dissolve LiOH and the mixture was stirred for 1 h 15 min. The mixture was cooled to rt, diluted with EtOAc and water and the pH was adjusted to pH ˜2-3. The product was extracted with EtOAc, dried over MgSO₄ and concentrated in vacuo to give 3-(5-methyltetrazol-2-yl)benzoic acid (260 mg, 99%, 1.2733 mmol) as a yellow powder. HPLC/MS m/z: 205.07, [M+H]⁺, Rt (T): 1.13 min.

Example 60.4.: 3-(5-methyltetrazol-2-yl)benzoic acid (264.00 mg, 1.2929 mmol), 2-(7-aza-1 H-benzotriazole-1-yl)-1,1,3,3-tetramethyluronium hexafluorophosphate (HATU) (688.25 mg, 1.8101 mmol) and tert-butyl (3S)-3-amino-6-[tert-butoxycarbonyl(methyl)amino]hexanoate (572.78 mg, 1.8101 mmol) were dissolved in dry DMF (6.46 mL). N,N-Diisopropylethylamine (0.68 mL, 3.8787 mmol) was added, and the reaction mixture was stirred at rt over 3 d. EtOAc and NaHCO₃ solution were added. The product was extracted with EtOAc, washed with water, dried over MgSO₄. Purification by silica gel column chromatography (eluent: 10-50% EtOAc in cyclohexane) to afford tert-butyl (3S)-6-[tert-butoxycarbonyl(methyl)-amino]-3-[[3-(5-methyltetrazol-2-yl)benzoyl]amino]hexanoate (570 mg, 88%, 1.1341 mmol) as a pale yellow powder. HPLC/MS m/z: 525.28, [M+Na]⁺, Rt (T): 1.50 min.

Example 60.5.: tert-Butyl (3S)-6-[tert-butoxycarbonyl(methyl)amino]-3-[[3-(5-methyltetrazol-2-yl)benzoyl]amino]hexanoate was used in analogous procedures to Example 152.6-Example 152.8 to afford (S)-N-(1-((5-(tert-butyl)-4-methylthiazol-2-yl)amino)-6-(methylamino)-1-oxohexan-3-yl)-3-(5-methyl-2H-tetrazol-2-yl)benzamide (36 mg, 56%, 0.0722 mmol) as a colorless powder. HPLC/MS m/z: 499.3, [M+H]⁺, Rt (U): 2.66 min. ¹H NMR (600 MHz, DMSO-d₆) δ 8.66 (d, J=8.5 Hz, 1H), 8.51 (t, J=1.9 Hz, 1H), 8.25-8.19 (m, 1H), 8.05-8.00 (m, 1H), 7.76 (t, J=7.9 Hz, 1H), 4.48-4.40 (m, 1H), 2.70-2.64 (m, 2H), 2.62 (s, 3H), 2.60-2.53 (m, 2H), 2.32 (s, 3H), 2.30 (s, 3H), 1.62-1.56 (m, 2H), 1.56-1.43 (m, 2H), 1.34 (s, 9H). (Note: 2×NH not observed).

### Example 61: Ethyl 2-(3-(3-(5-methyl-1,2,4-oxadiazol-3-yl)benzamido)propanamido)-thiazole-5-carboxylate

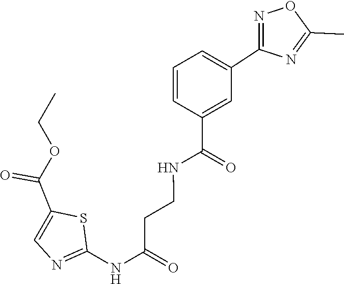 [SMILES: CCOC(=O)c1cnc(NC(=O)CCNC(=O)c2cccc(-c3noc(C)n3)c2)s1]

To a solution of ethyl 2-aminothiazole-5-carboxylate (37.11 mg, 0.2155 mmol), 3-[[3-(5-methyl-1,2,4-oxadiazol-3-yl)benzoyl]amino]propanoic acid [Example 63.3](50.00 mg, 0.1816 mmol) in DMF (0.91 mL) was added HOBt (49.09 mg, 0.3633 mmol) and EDC (56.40 mg, 0.3633 mmol). The mixture was stirred for 18 h at 60 0° C. The reaction mixture was partitioned between water (50 mL) and EtOAc (40 mL). The organic was washed with water (40 mL), 1 N HCl (20 mL), aqueous saturated bicarbonate (20 mL), and brine (20 mL). The organic layer was dried over sodium sulfate and concentrated. The crude purified by reverse phase chromatography (eluent: 30-100% MeOH in water (+0.1% formic acid modifier in both)) to afford ethyl 2-(3-(3-(5-methyl-1,2,4-oxadiazol-3-yl)benzamido)propan-amido)thiazole-5-carboxylate (10 mg, 13%, 0.0233 mmol) as a colorless, fluffy powder. HPLC/MS m/z: 430.1 [M+H]⁺, Rt (Q): 2.75 min. ¹H NMR (500 MHz, DMSO-d₆) δ 12.66 (s, 1H), 8.89 (t, J=5.5 Hz, 1H), 8.47 (t, J=1.7 Hz, 1H), 8.17-8.11 (m, 2H), 8.07-8.01 (m, 1H), 7.66 (t, J=7.8 Hz, 1H), 4.28 (q, J=7.1 Hz, 2H), 3.62 (q, J=6.5 Hz, 2H), 2.82 (t, J=6.8 Hz, 2H), 2.69 (s, 3H), 1.29 (t, J=7.1 Hz, 3H).

### Example 62: Methyl 4-methyl-2-(3-(3-(5-methyl-1,2,4-oxadiazol-3-yl)benzamido)propanamido)thiazole-5-carboxylate

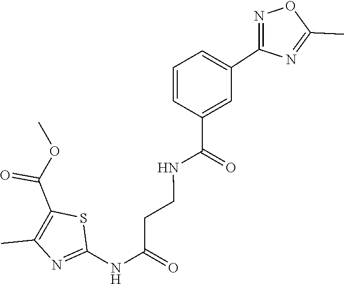

To 3-[[3-(5-methyl-1,2,4-oxadiazol-3-yl)benzoyl]amino]propanoic acid [Example 63.3](99.00 mg, 0.3597 mmol), methyl 2-amino-4-methyl-thiazole-5-carboxylate (73.49 mg, 0.4267 mmol) and HOBt (97.19 mg, 0.7193 mmol) in DMF (1.80 mL) under nitrogen atmosphere was added 3-(ethyliminomethyleneamino)-N,N-dimethylpropan-1-amine hydrochloride (137.89 mg, 0.7193 mmol). The solution was stirred for 18 h at 60 0° C. The reaction mixture was partitioned between water (50 mL) and EtOAc (40 mL). The organic was retained and the aqueous re-extracted with fresh EtOAc. The organics were combined, washed with aqueous saturated bicarbonate (30 mL), and brine (30 mL) and dried over MgSO₄. Concentration in vacuo gave a yellow crude (−150 mg). This was purified by reverse phases column chromatography (eluent: 30-100% MeOH in water (+0.1% formic acid modifier in both)) to afford methyl 4-methyl-2-(3-(3-(5-methyl-1,2,4-oxadiazol-3-yl)benzamido)propanamido)thiazole-5-carboxylate (40 mg, 26%, 0.0931 mmol) as a colorless powder. HPLC/MS m/z: 430.1 [M+H]⁺, Rt (Q): 3.08 min. ¹H NMR (500 MHz, DMSO-d₆) δ 12.66 (s, 1H), 8.89 (t, J=5.5 Hz, 1H), 8.47 (t, J=1.7 Hz, 1H), 8.17-8.11 (m, 2H), 8.07-8.01 (m, 1H), 7.66 (t, J=7.8 Hz, 1H), 4.28 (q, J=7.1 Hz, 2H), 3.62 (q, J=6.5 Hz, 2H), 2.82 (t, J=6.8 Hz, 2H), 2.69 (s, 3H), 1.29 (t, J=7.1 Hz, 3H).

### Example 63: Propyl 4-methyl-2-(3-(3-(5-methyl-1,2,4-oxadiazol-3-yl)benzamido)propanamido)thiazole-5-carboxylate

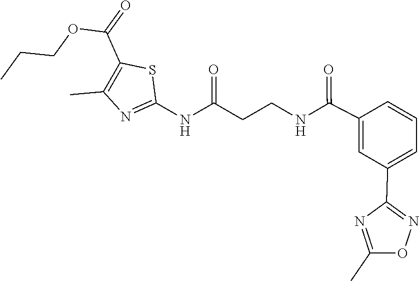 [SMILES: CCCOC(=O)c1sc(NC(=O)CCNC(=O)c2cccc(-c3noc(C)n3)c2)nc1C]

Example 63.1.: 2-Amino-4-methyl-thiazole-5-carboxylic acid (200.00 mg, 1.2644 mmol) and N,N-dimethylpyridin-4-amine (15.45 mg, 0.1264 mmol) were suspended in DMF (3.16 mL). EDC.HCl (290.86 mg, 1.5173 mmol) and propan-1-ol (1.90 mL, 25.288 mmol) were successively added and the solution was stirred at 60° C. for 1 h. Partitioned between EtOAc and water. Organics were washed with aqueous saturated bicarbonate and brine before drying over MgSO₄. Purified by silica gel column chromatography (eluent: 25-60% EtOAc in cyclohexane) to give propyl 2-amino-4-methyl-thiazole-5-carboxylate (115 mg, 45%, 0.5743 mmol) as an off-white powder. HPLC/MS m/z: 201.1 [M+H]⁺, Rt (R): 1.00 min.

Example 63.2.: To methyl 3-aminopropanoate hydrochloride (546.89 mg, 3.9181 mmol) and 3-(5-methyl-1,2,4-oxadiazol-3-yl)benzoic acid (800.00 mg, 3.9181 mmol) in DMF (19.59 mL) was added DIPEA (2.74 mL, 15.672 mmol) followed by HATU (1382.69 mg, 5.8772 mmol). Stirred at rt for 20 h. Diluted with EtOAc (200 mL) and washed with water (250 mL). The aqueous phase was extracted with fresh EtOAc (100 mL). The organics were combined and washed with aqueous saturated bicarbonate (150 mL), and brine (200 mL) before drying over MgSO₄. After filtering and concentrating in vacuo methyl 3-[[3-(5-methyl-1,2,4-oxadiazol-3-yl)benzoyl]amino]propanoate (1.12 g, 99%, 3.8715 mmol) was isolated as an off-white powder. HPLC/MS m/z: 312.1 [M+Na]⁺, Rt (R): 1.02 min.

Example 63.3.: To methyl 3-[[3-(5-methyl-1,2,4-oxadiazol-3-yl)benzoyl]amino]propanoate (1.12 g, 3.8715 mmol) in THF (19.26 mL) was added water (19.26 mL) followed by lithium hydroxide hydrate (0.65 g, 15.486 mmol). After stirring for 1 h 20 min the THE removed in vacuo. The solution was acidified to pH 3 with 1 M Citric acid solution and extracted with EtOAc (2×100 mL). The organics were combined, washed with brine (150 mL) and dried over MgSO₄. This gave 3-[[3-(5-methyl-1,2,4-oxadiazol-3-yl)benzoyl]amino]propanoic acid (917.3 mg, 86%, 3.3325 mmol) as a colorless solid. HPLC/MS m/z: 276.1 [M+H]⁺, Rt (R): 0.93 min.

Example 63.4.: Propyl 2-amino-4-methyl-thiazole-5-carboxylate (24.00 mg, 0.1198 mmol), 1-hydroxybenzotriazole (32.39 mg, 0.2397 mmol), 3-(ethyliminomethylene-amino)-N,N-dimethyl-propan-1-amine, HCl (37.21 mg, 0.2397 mmol) and 3-[[3-(5-methyl-1,2,4-oxadiazol-3-yl)benzoyl]amino]propanoic acid (42.88 mg, 0.1558 mmol) were dissolved in dry DMF (0.60 mL) at rt for 60 h. The reaction mixture was diluted with EtOAc and washed with NaHCO₃ (2×8 mL) and water (8 mL), dried over MgSO₄, and concentrated under reduced pressure. Purified by reverse phase column chromatography (eluent: 30-100% MeOH/H₂O+0.1% formic acid) afforded propyl 4-methyl-2-(3-(3-(5-methyl-1,2,4-oxadiazol-3-yl)benzamido)propan-amido)thiazole-5-carboxylate (30 mg, 55%, 0.0656 mmol) as an off-white solid. HPLC/MS m/z: 458.1 [M+H]⁺, Rt (S): 2.93 min. ¹H NMR (500 MHz, DMSO-d₆) δ 12.54 (s, 1H), 8.86 (t, J=5.5 Hz, 1H), 8.46 (t, J=1.7 Hz, 1H), 8.12 (dt, J=7.7, 1.4 Hz, 1H), 8.05-8.01 (m, 1H), 7.65 (t, J=7.8 Hz, 1H), 4.15 (t, J=6.5 Hz, 2H), 3.60 (q, J=6.6 Hz, 2H), 2.78 (t, J=6.8 Hz, 2H), 2.68 (s, 3H), 2.53 (s, 3H), 1.72-1.62 (m, 2H), 0.93 (t, J=7.4 Hz, 3H).

The following examples were prepared in a similar manner:

### Example 64: N-(3-((5-butyl-4-methylthiazol-2-yl)amino)-3-oxopropyl)-3-(5-methyl-1,2,4-oxadiazol-3-yl)benzamide

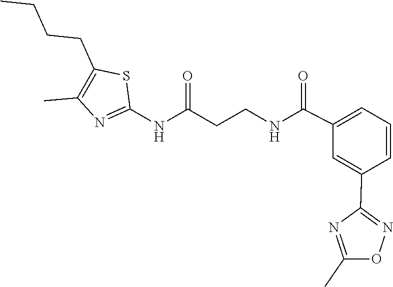 [SMILES: CCCCc1sc(NC(=O)CCNC(=O)c2cccc(-c3noc(C)n3)c2)nc1C]

31 mg (27%). HPLC/MS m/z: 428.18 [M+H]⁺, Rt (R): 1.44 min. ¹H NMR (500 MHz, DMSO-d₆) δ 11.90 (s, 1H), 8.85 (t, J=5.5 Hz, 1H), 8.47 (t, J=1.7 Hz, 1H), 8.12 (dt, J=7.7, 1.4 Hz, 1H), 8.03 (dt, J=7.9, 1.4 Hz, 1H), 7.65 (t, J=7.8 Hz, 1H), 3.57 (q, J=6.7 Hz, 2H), 2.69 (d, J=9.5 Hz, 5H), 2.63 (t, J=7.4 Hz, 2H), 2.14 (s, 3H), 1.57-1.42 (m, 2H), 1.39-1.24 (m, 2H), 0.88 (t, J=7.4 Hz, 3H).

### Example 65: Ethyl 4-methyl-2-(3-methyl-3-(3-(5-methyl-1,2,4-oxadiazol-3-yl)benzamido)butanamido)thiazole-5-carboxylate

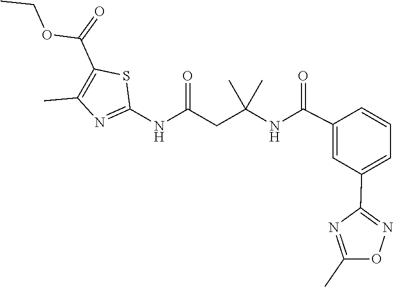 [SMILES: CCOC(=O)c1sc(NC(=O)CC(C)(C)NC(=O)c2cccc(-c3noc(C)n3)c2)nc1C]

Ethyl 3-amino-3-methyl-butanoate hydrochloride was used to prepare ethyl 4-methyl-2-(3-methyl-3-(3-(5-methyl-1,2,4-oxadiazol-3-yl)benzamido)butan-amido)thiazole-5-carboxylate (40 mg, 33%, 0.0848 mmol) as a colorless solid. HPLC/MS m/z: 472.17, [M+H]⁺, Rt (S): 2.98 min. ¹H NMR (500 MHz, DMSO-d₆) δ 12.46 (s, 1H), 8.38 (t, J=1.7 Hz, 1H), 8.22 (s, 1H), 8.11 (dt, J=7.8, 1.4 Hz, 1H), 7.97 (dt, J=7.9, 1.4 Hz, 1H), 7.64 (t, J=7.8 Hz, 1H), 4.21 (q, J=7.1 Hz, 2H), 3.07 (s, 2H), 2.68 (s, 3H), 2.52 (s, 3H), 1.48 (s, 6H), 1.26 (t, J=7.1 Hz, 3H).

### Example 66: Isopropyl 4-methyl-2-(3-(3-(5-methyl-1,2,4-oxadiazol-3-yl)benzamido)propanamido)thiazole-5-carboxylate

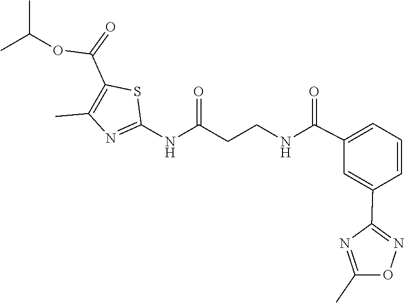 [SMILES: Cc1nc(-c2cccc(C(=O)NCCC(=O)Nc3nc(C)c(C(=O)OC(C)C)s3)c2)no1]

Example 66.1.: Prepared in an analogous procedure to Example 63 using 2-amino-4-methyl-thiazole-5-carboxylic acid to afford 4-methyl-2-[3-[[3-(5-methyl-1,2,4-oxadiazol-3-yl)benzoyl]amino]propanoylamino]thiazole-5-carboxylic acid (84 mg, 62%, 0.2022 mmol) as a brown powder. HPLC/MS m/z: 416.1 [M+H]⁺, Rt (R): 1.13 min.

Example 66.2.: 4-methyl-2-[3-[[3-(5-methyl-1,2,4-oxadiazol-3-yl)benzoyl]-amino]propanoylamino]thiazole-5-carboxylic acid (40.00 mg, 0.0963 mmol) was dissolved in dry DCM (1.50 mL). Stirred at rt for 6 h before the solvent was removed under reduced pressure. The residue was dissolved in dry i-PrOH and stirred at 50° C. overnight. The reaction mixture was concentrated, and the residue purified by reverse phase column chromatography (eluent: 30-100% MeOH/H₂O+0.1% formic acid) to afford isopropyl 4-methyl-2-(3-(3-(5-methyl-1,2,4-oxadiazol-3-yl)benz-amido)propanamido)thiazole-5-carboxylate (22 mg, 50%, 0.0481 mmol) as a colorless powder. HPLC/MS m/z: 458.2 [M+H]⁺, Rt (S): 2.90 min. ¹H NMR (500 MHz, DMSO-d₆) δ 12.52 (s, 1H), 8.86 (t, J=5.5 Hz, 1H), 8.47-8.44 (m, 1H), 8.13 (ddd, J=7.7, 1.7, 1.2 Hz, 1H), 8.02 (ddd, J=7.8, 1.9, 1.2 Hz, 1H), 7.65 (td, J=7.7, 0.5 Hz, 1H), 5.05 (hept, J=6.3 Hz, 1H), 3.63-3.57 (m, 2H), 2.78 (t, J=6.8 Hz, 2H), 2.68 (s, 3H), 2.53 (s, 3H), 1.28 (d, J=6.2 Hz, 6H).

The following examples were prepared analogously.

### Example 67: 2-Methoxyethyl 4-methyl-2-(3-(3-(5-methyl-1,2,4-oxadiazol-3-yl)benzamido)propanamido)thiazole-5-carboxylate

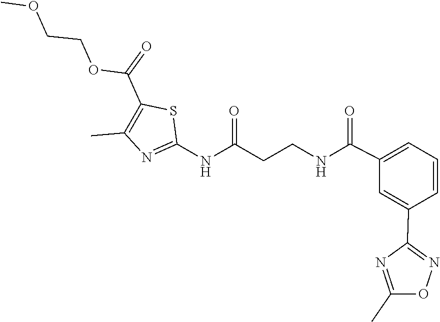 [SMILES: COCCOC(=O)c1sc(NC(=O)CCNC(=O)c2cccc(-c3noc(C)n3)c2)nc1C]

23 mg (50%) colorless, amorphous solid. HPLC/MS m/z: 474.1 [M+H]⁺, Rt (S): 2.59 min. ¹H NMR (500 MHz, DMSO-d₆) δ 12.55 (s, 1H), 8.86 (t, J=5.5 Hz, 1H), 8.46 (td, J=1.8, 0.5 Hz, 1H), 8.13 (ddd, J=7.7, 1.8, 1.2 Hz, 1H), 8.02 (ddd, J=7.9, 1.9, 1.2 Hz, 1H), 7.65 (td, J=7.7, 0.6 Hz, 1H), 4.34-4.28 (m, 2H), 3.63-3.55 (m, 4H), 3.29 (s, 3H), 2.79 (t, J=6.8 Hz, 2H), 2.68 (s, 3H), 2.54 (s, 3H).

### Example 68: Isobutyl 4-methyl-2-(3-(3-(5-methyl-1,2,4-oxadiazol-3-yl)benzamido)propanamido)thiazole-5-carboxylate

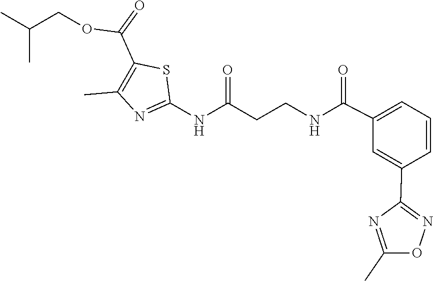 [SMILES: Cc1nc(-c2cccc(C(=O)NCCC(=O)Nc3nc(C)c(C(=O)OCC(C)C)s3)c2)no1]

18 mg (40%) colorless powder. HPLC/MS m/z: 472.2 [M+H]⁺, Rt (S): 3.06 min. ¹H NMR (500 MHz, DMSO-d₆) δ 12.55 (s, 1H), 8.87 (t, J=5.5 Hz, 1H), 8.46 (t, J=1.7 Hz, 1H), 8.13 (dt, J=7.7, 1.4 Hz, 1H), 8.03 (dt, J=7.9, 1.4 Hz, 1H), 7.65 (t, J=7.8 Hz, 1H), 4.00 (d, J=6.4 Hz, 2H), 3.66-3.55 (m, 2H), 2.78 (t, J=6.8 Hz, 2H), 2.68 (s, 3H), 2.54 (s, 3H), 2.02-1.91 (m, 1H), 0.94 (d, J=6.7 Hz, 6H).

### Example 69: 4-Methoxybutyl 4-methyl-2-(3-(3-(5-methyl-1,2,4-oxadiazol-3-yl)benzamido)propanamido)thiazole-5-carboxylate

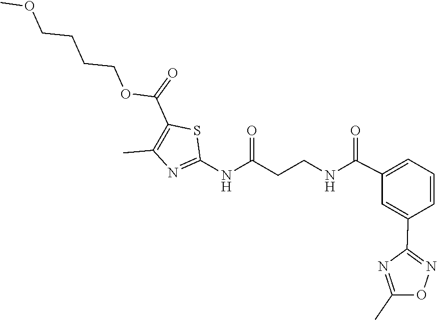 [SMILES: COCCCCOC(=O)c1sc(NC(=O)CCNC(=O)c2cccc(-c3noc(C)n3)c2)nc1C]

30 mg (62%) colorless powder. HPLC/MS m/z: 502.2 [M+H]⁺, Rt (S): 2.83 min. ¹H NMR (500 MHz, DMSO-d₆) δ 12.54 (s, 1H), 8.86 (t, J=5.5 Hz, 1H), 8.47-8.43 (m, 1H), 8.15-8.10 (m, 1H), 8.05-8.00 (m, 1H), 7.69-7.62 (m, 1H), 4.20 (t, J=6.4 Hz, 2H), 3.63-3.56 (m, 2H), 3.35 (t, J=6.3 Hz, 2H), 3.22 (s, 3H), 2.78 (t, J=6.8 Hz, 2H), 2.68 (s, 3H), 2.53 (s, 3H), 1.73-1.65 (m, 2H), 1.62-1.55 (m, 2H).

### Example 70: Propyl 4-methyl-2-((2-((7-(5-methyl-1,2,4-oxadiazol-3-yl)isoquinolin-1-yl)amino)ethyl)carbamoyl)thiazole-5-carboxylate

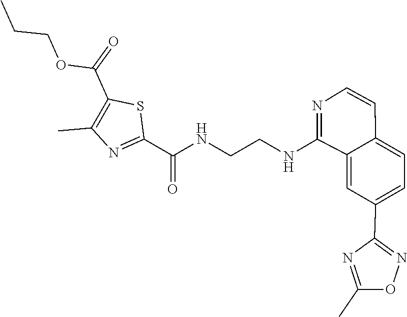 [SMILES: CCCOC(=O)c1sc(C(=O)NCCNc2nccc3ccc(-c4noc(C)n4)cc23)nc1C]

Example 70.1.: A solution of 3-(1-chloro-7-isoquinolyl)-5-methyl-1,2,4-oxadiazole (60.00 mg, 0.2442 mmol) and ethylenediamine (0.33 mL, 4.8848 mmol) in NMP (0.98 mL) in a 0.5-2 mL microwave vial was heated under microwave irradiation for 1 h at 160° C. Volatiles were removed under reduced pressure. Residue was added to a saturated bicarbonate solution (15 mL) and extracted with EtOAc (3×10 mL). Organic layer was combined, washed with brine (15 mL), dried over magnesium sulfate, and concentrated in vacuo. Purification by Biotage KP-NH silica gel column chromatography (eluent: 0-15% (EtOAc/MeOH—3/1) in DCM) to afford N′-[7-(5-methyl-1,2,4-oxadiazol-3-yl)-1-isoquinolyl]ethane-1,2-diamine (48.8 mg, 74%, 0.1812 mmol) as a colorless solid. HPLC/MS m/z: 270.13, [M+H]⁺, Rt (P): 0.23 min.

Example 70.2.: To a mixture of N′-[7-(5-methyl-1,2,4-oxadiazol-3-yl)-1-isoquinolyl]-ethane-1,2-diamine (50.75 mg, 0.1884 mmol), 4-methyl-5-propoxycarbonyl-thiazole-2-carboxylic acid [Example 74](36.00 mg, 0.1570 mmol), EDC.HCl (60.21 mg, 0.3141 mmol) and 1-hydroxybenzotriazole (42.44 mg, 0.3141 mmol) was added under nitrogen atmosphere DMF (0.79 mL). The solution was stirred at rt for 3 h. LCMS showed 50% conversion. The solution was stirred at rt over the weekend. LCMS showed complete conversion. The solution was added to water (20 mL) and extracted with EtOAc (3×10 mL). Organic layer was combined, washed with a saturated bicarbonate solution (15 mL), with brine (15 mL), dried over magnesium sulfate and concentrated under reduced pressure. Purification by silica gel column chromatography (eluent: 20-50% EtOAc in cyclohexane) followed by reverse phase column chromatography (eluent: 10-100% MeOH in water (+0.1% formic acid in both)). Purification by ion exchange SCX-II column chromatography, washing with MeOH before eluting with 2M NH₃ in MeOH afforded propyl 4-methyl-2-((2-((7-(5-methyl-1,2,4-oxadiazol-3-yl)isoquinolin-1-yl)amino)ethyl)carbamoyl)thiazole-5-carboxylate (29 mg, 38%, 0.0603 mmol) as a colorless solid. HPLC/MS m/z: 481.16, [M+H]⁺, Rt (S): 2.61 min. ¹H NMR (600 MHz, DMSO-d₆) δ 9.37 (t, J=5.5 Hz, 1H), 8.89-8.87 (m, 1H), 8.15 (dd, J=8.5, 1.5 Hz, 1H), 8.03 (t, J=5.5 Hz, 1H), 7.99 (d, J=5.7 Hz, 1H), 7.85 (d, J=8.5 Hz, 1H), 7.00-6.96 (m, 1H), 4.22 (t, J=6.5 Hz, 2H), 3.72 (q, J=5.6 Hz, 2H), 3.59 (q, J=5.7 Hz, 2H), 2.71-2.68 (m, 6H), 1.73-1.65 (m, 2H), 0.94 (t, J=7.4 Hz, 3H).

### Example 71: Ethyl 1-methyl-3-((2-((7-(5-methyl-1,2,4-oxadiazol-3-yl)isoquinolin-1-yl)amino)ethyl)carbamoyl)-1 H-pyrazole-5-carboxylate

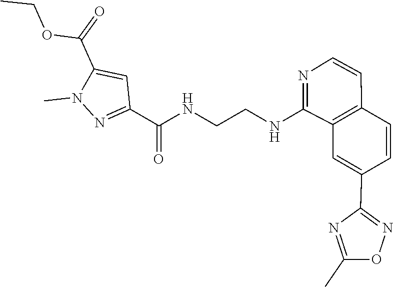 [SMILES: CCOC(=O)c1cc(C(=O)NCCNc2nccc3ccc(-c4noc(C)n4)cc23)nn1C]

To a stirring solution containing 1-methyl-1 H-pyrazole-3,5-dicarboxylic acid 5-ethyl ester (65.00 mg, 0.3280 mmol), HATU (187.06 mg, 0.4920 mmol) and DIPEA (0.11 mL, 0.6560 mmol) in DMF (0.82 mL) in a LCMS vial was added N′-[7-(5-methyl-1,2,4-oxadiazol-3-yl)-1-isoquinolyl]ethane-1,2-diamine [Example 70](97.16 mg, 0.3608 mmol). The resulting solution was stirred at rt overnight. Purified by reverse phase column chromatography (eluent: 20-70% MeOH in water (+0.1% formic acid in both)) followed by ion exchange SCX-II column chromatography, washing with MeOH before eluting with a mixture of DCM/NH₃ 7M in MeOH (1/1) to afford ethyl 1-methyl-3-((2-((7-(5-methyl-1,2,4-oxadiazol-3-yl)isoquinolin-1-yl)amino)ethyl)-carbamoyl)-1 H-pyrazole-5-carboxylate (94 mg, 64%, 0.2091 mmol) as a colorless solid. HPLC/MS m/z: 450.19, [M+H]⁺, Rt (S): 2.12 min. ¹H NMR (600 MHz, DMSO-d₆) δ 8.90-8.86 (m, 1H), 8.78 (t, J=5.7 Hz, 1H), 8.15 (dd, J=8.5, 1.5 Hz, 1H), 8.00 (t, J=5.5 Hz, 1H), 7.96 (d, J=5.7 Hz, 1H), 7.85 (d, J=8.5 Hz, 1H), 7.37 (s, 1H), 6.99-6.96 (m, 1H), 4.27 (q, J=7.1 Hz, 2H), 4.13 (s, 3H), 3.68 (q, J=6.1 Hz, 2H), 3.54 (q, J=6.1 Hz, 2H), 2.70 (s, 3H), 1.28 (t, J=7.1 Hz, 3H).

### Example 72: Propyl 1-methyl-3-((2-((7-(5-methyl-1,2,4-oxadiazol-3-yl)isoquinolin-1-yl)amino)ethyl)carbamoyl)-1 H-pyrazole-5-carboxylate

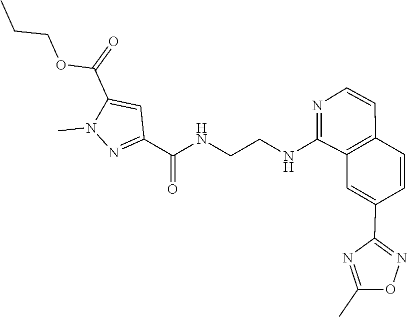 [SMILES: CCCOC(=O)c1cc(C(=O)NCCNc2nccc3ccc(-c4noc(C)n4)cc23)nn1C]

Example 72.1.: Ethyl 1-methyl-3-(4-((7-(5-methyl-1,2,4-oxadiazol-3-yl)isoquinolin-1-yl)amino)butanoyl)-1 H-pyrazole-5-carboxylate (65.00 mg, 0.1446 mmol) was dissolved at rt in THF (0.75 mL) and water (0.75 mL) and hydroxylithium hydrate (24.27 mg, 0.5785 mmol) was added. The resulting solution was heated at 70° C. for 4 h. The solution was cooled down to rt and volatiles were removed under reduced pressure. Water (2 mL) was added, and the solution was then acidified by adding few drops of HCl 37% until a pH around 1 was reached. Solid crashed out in the solution, filtered off, washed with water (5 mL) and dried under the high vacuum to afford 2-methyl-5-[2-[[7-(5-methyl-1,2,4-oxadiazol-3-yl)-1-isoquinolyl]amino]-ethylcarbamoyl]pyrazole-3-carboxylic acid (45 mg, 74%, 0.1068 mmol) as a colorless solid. HPLC/MS m/z: 422.15, [M+H]⁺, Rt (P): 1.11 min.

Example 72.2.: 2-Methyl-5-[2-[[7-(5-methyl-1,2,4-oxadiazol-3-yl)-1-isoquinolyl]amino]ethylcarbamoyl]pyrazole-3-carboxylic acid (20.00 mg, 0.0475 mmol) and DMAP (1.16 mg, 0.0095 mmol) were dissolved in dry DCM (0.16 mL) at rt. 1 M DCC (0.06 mL, 0.0570 mmol) and propan-1-ol (0.04 mL, 0.4746 mmol) were successively added and the reaction mixture was stirred at rt overnight. DCM was removed under reduced pressure and the residue purified by reverse phase column chromatography (eluent: 20-70% MeOH in water (+0.1% formic acid in both) followed by purification by ion exchange SCX-II column chromatography, washing with MeOH before eluting with 2M NH₃ in MeOH to afford (10 mg, 45%, 0.0216 mmol) propyl 1-methyl-3-((2-((7-(5-methyl-1,2,4-oxadiazol-3-yl)isoquinolin-1-yl)amino)ethyl)carbamoyl)-1 H-pyrazole-5-carboxylate as a colorless solid. HPLC/MS m/z: 464.20, [M+H]⁺, Rt (U): 2.29 min. ¹H NMR (600 MHz, DMSO-d₆) δ 8.91-8.87 (m, 1H), 8.79 (t, J=5.7 Hz, 1H), 8.15 (dd, J=8.5, 1.5 Hz, 1H), 8.01 (t, J=5.6 Hz, 1H), 7.96 (d, J=5.7 Hz, 1H), 7.85 (d, J=8.5 Hz, 1H), 7.37 (s, 1H), 6.99-6.94 (m, 1H), 4.18 (t, J=6.6 Hz, 2H), 4.13 (s, 3H), 3.69 (q, J=6.1 Hz, 2H), 3.54 (q, J=6.1 Hz, 2H), 2.70 (s, 3H), 1.68 (sext, J=7.1 Hz, 2H), 0.93 (t, J=7.4 Hz, 3H).

### Example 73: 1-Methyl-3-(3-((7-(5-methyl-1,2,4-oxadiazol-3-yl)isoquinolin-1-yl)amino)propanamido)-N-propyl-1 H-pyrazole-5-carboxamide

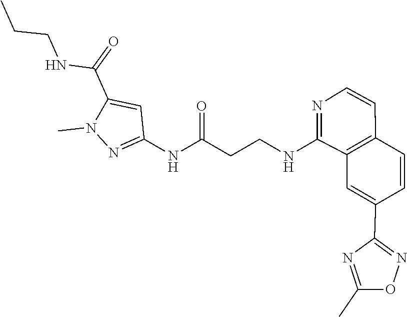 [SMILES: CCCNC(=O)c1cc(NC(=O)CCNc2nccc3ccc(-c4noc(C)n4)cc23)nn1C]

A solution of 2-methyl-5-[2-[[7-(5-methyl-1,2,4-oxadiazol-3-yl)-1-isoquinolyl]amino]-ethylcarbamoyl]pyrazole-3-carboxylic acid formate [Example 72.1](30.00 mg, 0.0642 mmol), HOBt (19.64 mg, 0.1284 mmol) and propylamine (0.03 mL, 0.3209 mmol) in DMF (0.64 mL) was stirred at rt for 16 h. 2 eq of EDC.HCl, 2 eq of HOBt and 5 eq of propylamine were added and the resulting solution was stirred at 40° C. for 4 h. Cooled down to rt and purified by reverse phase column chromatography (eluent: 20-80% MeOH in water (+0.1% formic acid) followed by ion exchange SCX-II column chromatography, washing with MeOH before eluting with 2 M NH₃ in MeOH to afford 1-methyl-3-(3-((7-(5-methyl-1,2,4-oxadiazol-3-yl)isoquinolin-1-yl)amino)propanamido)-N-propyl-1 H-pyrazole-5-carboxamide (10.4 mg, 35%, 0.0225 mmol) as a colorless solid. HPLC/MS m/z: 463.22, [M+H]⁺, Rt (U): 2.11 min. ¹H NMR (600 MHz, DMSO-d₆) δ 10.57 (s, 1H), 8.90-8.87 (m, 1H), 8.52 (t, J=5.7 Hz, 1H), 8.15 (dd, J=8.5, 1.6 Hz, 1H), 7.99 (d, J=5.7 Hz, 1H), 7.94 (t, J=5.4 Hz, 1H), 7.85 (d, J=8.5 Hz, 1H), 7.16 (s, 1H), 6.97 (d, J=5.7 Hz, 1H), 3.95 (s, 3H), 3.78 (td, J=7.1, 5.3 Hz, 2H), 3.15 (dt, J=7.3, 6.0 Hz, 2H), 2.74 (t, J=7.1 Hz, 2H), 2.71 (s, 3H), 1.51 (sext, J=7.3 Hz, 2H), 0.87 (t, J=7.4 Hz, 3H).

### Example 74: Propyl 4-methyl-2-(methyl(2-((7-(5-methyl-1,2,4-oxadiazol-3-yl)isoquinolin-1-yl)amino)ethyl)carbamoyl)thiazole-5-carboxylate

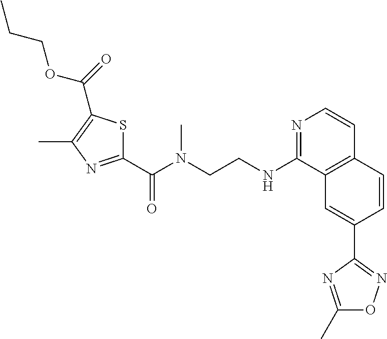 [SMILES: CCCOC(=O)c1sc(C(=O)N(C)CCNc2nccc3ccc(-c4noc(C)n4)cc23)nc1C]

Example 74.1.: A solution of 3-(1-chloro-7-isoquinolyl)-5-methyl-1,2,4-oxadiazole [Example 45](50.00 mg, 0.2035 mmol) and N-(2-aminoethyl)-N-methyl carbamic acid tert-butylester (0.73 mL, 4.0707 mmol) in NMP (0.81 mL) in a 0.5-2 mL microwave vial was heated under microwave irradiation for 1 h at 160 0° C. Residue was added to water (20 mL) and extracted with EtOAc (3×15 mL). Organic layer was combined, washed with brine (20 mL), dried over magnesium sulfate and concentrated in vacuo. Purified by reverse phase column chromatography (eluent: 20-70% MeOH in water (+0.1% formic acid in both)) to afford tert-butyl N-methyl-N-[2-[[7-(5-methyl-1,2,4-oxadiazol-3-yl)-1-isoquinolyl]amino]ethyl]carbamate (60 mg, 77%, 0.1565 mmol) as a light-yellow oil. HPLC/MS m/z: 384.20, [M+H]⁺, Rt (P): 1.25 min.

Example 74.2.: tert-Butyl N-methyl-N-[2-[[7-(5-methyl-1,2,4-oxadiazol-3-yl)-1-isoquinolyl]amino]ethyl]carbamate (55.00 mg, 0.1434 mmol) was dissolved in dioxane (0.48 mL), 4 M HCl in dioxane (0.72 mL, 2.8688 mmol) was added dropwise, while stirring at rt. The mixture was stirred 5.5 h at rt. Purification by ion exchange SCX-II column chromatography, washing with MeOH before eluting with 2M NH₃ in MeOH afforded N-methyl-N′-[7-(5-methyl-1,2,4-oxadiazol-3-yl)-1-isoquinolyl]ethane-1,2-diamine (30 mg, 74%, 0.1059 mmol) as a colorless powder. HPLC/MS m/z: 284.15, [M+H]⁺, Rt (P): 0.31 min.

Example 74.3.: 2-Bromo-4-methyl-thiazole-5-carboxylic acid (530.00 mg, 2.3867 mmol) and N,N-dimethylpyridin-4-amine (29.16 mg, 0.2387 mmol) were suspended in DMF (11.93 mL). EDC.HCl (549.05 mg, 2.8641 mmol) and propan-1-ol (3.59 mL, 47.735 mmol) were successively added and the solution was stirred at 60° C. for 1 h. The solution was then cooled down to rt, added to water (75 mL), extracted with EtOAc (3×50 mL). Organic layers were combined, washed with water (70 mL), a saturated bicarbonate solution (70 mL), a 1 M HCl solution (70 mL), brine (70 mL), dried over magnesium sulfate and concentrated under reduced pressure. Purification by silica gel column chromatography (eluent: 0-40% (cyclohexane/EtOAc—9/1) in cyclohexane) to afford propyl 2-bromo-4-methyl-thiazole-5-carboxylate (410 mg, 65%, 1.5522 mmol) as a colorless solid. HPLC/MS m/z: 265.97, [M+H]⁺, Rt (P): 1.55 min.

Example 74.4.: A solution of isopropylmagnesium chloride—lithium chloride complex (1.53 mL, 1.9933 mmol) was added to a solution of propyl 2-bromo-4-methyl-thiazole-5-carboxylate (405.00 mg, 1.5333 mmol) in dry THF (10.00 mL) at −78° C. The resulting solution was stirred for 10 min at −78° C. and then 4-formyl morpholine (0.39 mL, 3.8332 mmol) was added. After 15 min of stirring the solution was quenched with a saturated NH₄Cl solution (20 mL) and extracted with EtOAc (2×15 mL). Organic layers were combined, dried over magnesium sulfate and concentrated under reduced pressure. Purification by silica gel column chromatography (eluent: 0-40% of a mixture (cyclohexane/EtOAc—9/1) in cyclohexane afforded propyl 2-formyl-4-methyl-thiazole-5-carboxylate (186 mg, 57%, 0.8722 mmol) as a colourless oil. HPLC/MS m/z: 214.05, [M+H]⁺, Rt (T): 1.35 min.

Example 74.5.: t-BuOH (2.85 mL) was added to a solution of propyl 2-formyl-4-methyl-thiazole-5-carboxylate (80.00 mg, 0.3751 mmol) and 2-methyl-2-butene (1.19 mL, 11.254 mmol) in THF (3.01 mL). The resulting solution was stirred at rt for 10 min. A freshly prepared solution of sodium chlorite (111.96 mg, 1.238 mmol) and sodium dihydrogen phosphate hydrate (155.30 mg, 1.1254 mmol) in water (0.56 mL) was added and the solution was stirred at rt for 15 min. Solution was quenched with a saturated Na₂S₂O₃ solution (20 mL) and extracted with EtOAc (3×15 mL). The organic layer was dried over magnesium sulfate and concentrated under reduced pressure to afford 4-methyl-5-propoxycarbonyl-thiazole-2-carboxylic acid (44 mg, 51%, 0.1919 mmol) as colourless oil which was quickly used in the next step without further purification. HPLC/MS m/z: 230.05, [M+H]⁺, Rt (T): 1.14 min.

Example 74.6.: To a stirring solution containing 4-methyl-5-propoxycarbonyl-thiazole-2-carboxylic acid (24.27 mg, 0.1059 mmol), N-methyl-N′-[7-(5-methyl-1,2,4-oxadiazol-3-yl)-1-isoquinolyl]ethane-1,2-diamine (30.00 mg, 0.1059 mmol) and HATU (60.39 mg, 0.1588 mmol) in DMF (0.53 mL) was added DIPEA (0.04 mL, 0.2118 mmol). The resulting solution was stirred at rt for 4.5 h. The solution was added to water (15 mL) and the residue was extracted with EtOAc (3×10 mL). Purification by silica gel column chromatography (eluent: 20-50% EtOAc in cyclohexane) followed by reverse phase column chromatography (eluent: 20-70% MeOH in water (+0.1% formic acid in both)). Purification by ion exchange SCX-II column chromatography, washing with MeOH before eluting with DCM/NH₃ 7M in MeOH (1/1) afforded propyl 4-methyl-2-(methyl(2-((7-(5-methyl-1,2,4-oxadiazol-3-yl)isoquinolin-1-yl)amino)ethyl)carbamoyl)thiazole-5-carboxylate (24.2 mg, 46%, 0.0489 mmol) as a colorless solid. HPLC/MS m/z: 495.18, [M+H]⁺, Rt (P): 2.55 min. ¹H NMR (600 MHz, DMSO-d₆) δ 8.57 (s, 1H), 8.05 (dd, J=8.4, 1.5 Hz, 1H), 7.89 (t, J=5.7 Hz, 1H), 7.84 (d, J=5.7 Hz, 1H), 7.69 (d, J=8.5 Hz, 1H), 6.82 (d, J=5.7 Hz, 1H), 4.36-4.31 (m, 2H), 3.94 (t, J=6.6 Hz, 2H), 3.61 (q, J=5.2 Hz, 2H), 3.13 (s, 3H), 2.71 (s, 3H), 2.25 (s, 3H), 1.50 (hept, J=6.8 Hz, 2H), 0.82 (t, J=7.4 Hz, 3H).

### Example 75: N-(5-(tert-butyl)-4-methylthiazol-2-yl)-3-((7-(5-methyl-1,2,4-oxadiazol-3-yl)isoquinolin-1-yl)amino)propenamide

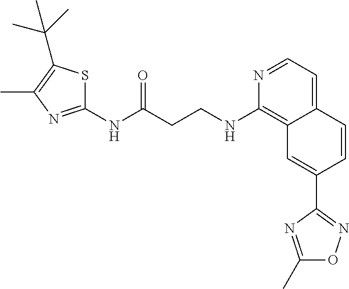 [SMILES: Cc1nc(-c2ccc3ccnc(NCCC(=O)Nc4nc(C)c(C(C)(C)C)s4)c3c2)no1]

Example 75.1.: To sodium 3-aminopropanoate (795.82 mg, 7.1644 mmol) in a 50 mL RBF fitted with an air condenser was added NMP (5.97 mL). The mixture was heated to 100° C. and stirred until all solid was dissolved. 3-(1-chloro-7-isoquinolyl)-5-methyl-1,2,4-oxadiazole [0](440.00 mg, 1.7911 mmol) was then added and the solution was stirred at 100° C. overnight under a nitrogen atmosphere. After cooling to rt, water (6 mL) was added. The obtained solution was directly purified by reverse phase column chromatography (eluent: 20-70% MeOH in water (+0.1% formic acid modifier in both)) to afford 3-[[7-(5-methyl-1,2,4-oxadiazol-3-yl)-1-isoquinolyl]amino]propanoic acid (503 mg, 94%, 1.6862 mmol) as a beige powder. HPLC/MS m/z: 299.11, [M+H]⁺, Rt (R): 0.78 min.

Example 75.2.: To 3-[[7-(5-methyl-1,2,4-oxadiazol-3-yl)-1-isoquinolyl]amino]-propanoic acid (20.00 mg, 0.0670 mmol) was added 5-tert-butyl-4-methyl-thiazol-2-ylamine (34.25 mg, 0.2011 mmol), PyBrop (75.01 mg, 0.1609 mmol) followed by dry DMF (0.34 mL) and DIPEA (42.04 uL, 0.2414 mmol). The resulting solution was stirred at rt overnight. Water was added (2 mL) and the residue was extracted with DCM (3×1 mL) using small separator phase column. Organic layer was combined and concentrated under reduced pressure. Purification by silica gel column chromatography (eluent: 20-60% EtOAc in cyclohexane) followed by purifcation by reverse phase column chromatography (eluent: 30-80% MeOH in water (+0.1% formic acid in both)). Ion exchange SCX-II column chromatography, washing with MeOH before eluting with 2M NH₃ in MeOH afforded N-(5-(tert-butyl)-4-methylthiazol-2-yl)-3-((7-(5-methyl-1,2,4-oxadiazol-3-yl)isoquinolin-1-yl)amino)propanamide (15.7 mg, 52%, 0.0348 mmol) as a colorless solid. HPLC/MS m/z: 452.19, [M+H]⁺, Rt (Q): 2.69 min. ¹H NMR (500 MHz, DMSO-d₆) δ 11.87 (s, 1H), 8.93-8.85 (m, 1H), 8.15 (dd, J=8.5, 1.6 Hz, 1H), 8.03-7.95 (m, 2H), 7.86 (d, J=8.4 Hz, 1H), 6.98 (dd, J=5.9, 0.9 Hz, 1H), 3.83-3.75 (m, 2H), 2.81 (t, J=6.9 Hz, 2H), 2.71 (s, 3H), 2.30 (s, 3H), 1.36 (s, 9H).

The following examples were synthesised by an analogous procedure:

### Example 76: N-(5-ethyl-1-methyl-1 H-pyrazol-3-yl)-3-((7-(5-methyl-1,2,4-oxadiazol-3-yl)isoquinolin-1-yl)amino)propenamide

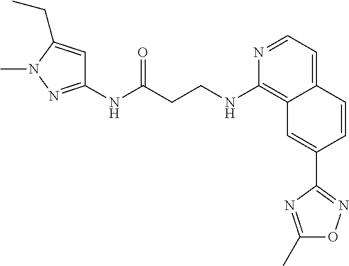 [SMILES: CCc1cc(NC(=O)CCNc2nccc3ccc(-c4noc(C)n4)cc23)nn1C]

7 mg (30%) pale-yellow solid. HPLC/MS m/z: 406.20, [M+H]⁺, Rt (S): 2.04 min. ¹H NMR (600 MHz, DMSO-d₆) δ 10.28 (s, 1H), 8.90-8.87 (m, 1H), 8.14 (dd, J=8.5, 1.5 Hz, 1H), 7.98 (d, J=5.7 Hz, 1H), 7.93 (t, J=5.4 Hz, 1H), 7.84 (d, J=8.5 Hz, 1H), 6.95 (d, J=5.7 Hz, 1H), 6.32 (s, 1H), 3.74 (td, J=7.1, 5.3 Hz, 2H), 3.59 (s, 3H), 2.69 (d, J=8.3 Hz, 5H), 2.56 (q, J=7.5 Hz, 2H), 1.16 (t, J=7.5 Hz, 3H).

### Example 77: N-(5-(tert-butyl)isoxazol-3-yl)-3-((7-(5-methyl-1,2,4-oxadiazol-3-yl)isoquinolin-1-yl)amino)propenamide

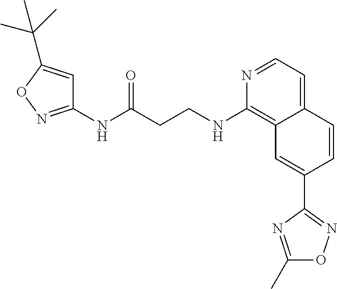 [SMILES: Cc1nc(-c2ccc3ccnc(NCCC(=O)Nc4cc(C(C)(C)C)on4)c3c2)no1]

10 mg (41%) colorless solid. HPLC/MS m/z: 421.20, [M+H]⁺, Rt (S): 2.41 min. ¹H NMR (600 MHz, DMSO-d₆) δ 10.98 (s, 1H), 8.92-8.83 (m, 1H), 8.14 (dd, J=8.5, 1.5 Hz, 1H), 7.97 (d, J=5.7 Hz, 1H), 7.95 (t, J=5.4 Hz, 1H), 7.85 (d, J=8.5 Hz, 1H), 7.00-6.92 (m, 1H), 6.61 (s, 1H), 3.80-3.74 (m, 2H), 2.77 (t, J=6.9 Hz, 2H), 2.70 (s, 3H), 1.28 (s, 9H).

### Example 78: N-(5-cyclopropylisoxazol-3-yl)-3-((7-(5-methyl-1,2,4-oxadiazol-3-yl)isoquinolin-1-yl)amino)propenamide

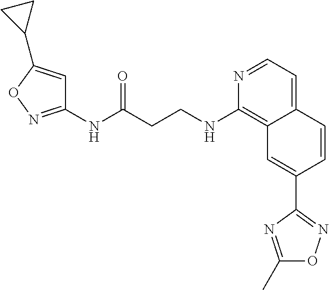 [SMILES: Cc1nc(-c2ccc3ccnc(NCCC(=O)Nc4cc(C5CC5)on4)c3c2)no1]

9 mg (41%) colorless solid. HPLC/MS m/z: 405.17, [M+H]⁺, Rt (P): 1.21 min. ¹H NMR (600 MHz, DMSO-d₆) δ 10.92 (s, 1H), 8.90-8.84 (m, 1H), 8.14 (dd, J=8.5, 1.5 Hz, 1H), 7.97 (d, J=5.8 Hz, 1H), 7.95 (d, J=5.4 Hz, 1H), 7.84 (d, J=8.5 Hz, 1H), 6.96 (d, J=5.7 Hz, 1H), 6.58 (s, 1H), 3.76 (td, J=6.9, 5.2 Hz, 2H), 2.77 (t, J=6.9 Hz, 2H), 2.70 (s, 3H), 2.10 (tt, J=8.5, 5.0 Hz, 1H), 1.07-0.99 (m, 2H), 0.90-0.82 (m, 2H).

### Example 79: N-(5-ethylisoxazol-3-yl)-3-((7-(5-methyl-1,2,4-oxadiazol-3-yl)isoquinolin-1-yl)amino)propanamide

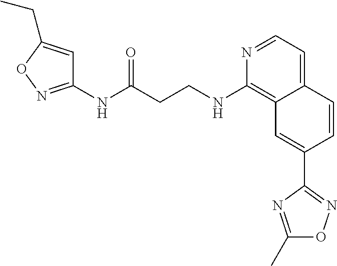 [SMILES: CCc1cc(NC(=O)CCNc2nccc3ccc(-c4noc(C)n4)cc23)no1]

1 mg (4%) colourless oil. HPLC/MS m/z: 393.17, [M+H]⁺, Rt (S): 2.10 min. ¹H NMR (600 MHz, DMSO-d₆) δ 11.56 (s, 1H), 8.90-8.86 (m, 1H), 8.15 (dd, J=8.5, 1.5 Hz, 1H), 7.99 (d, J=5.4 Hz, 1H), 7.97 (d, J=5.8 Hz, 1H), 7.85 (d, J=8.5 Hz, 1H), 6.97 (d, J=5.7 Hz, 1H), 6.17 (s, 1H), 3.78 (q, J=6.6 Hz, 2H), 2.80 (t, J=6.8 Hz, 2H), 2.70 (s, 3H), 2.56 (q, J=7.6 Hz, 2H), 1.16 (t, J=7.6 Hz, 3H).

### Example 80: N-(5-(tert-butyl)thiazol-2-yl)-3-((7-(5-methyl-1,2,4-oxadiazol-3-yl)isoquinolin-1-yl)amino)propanamide

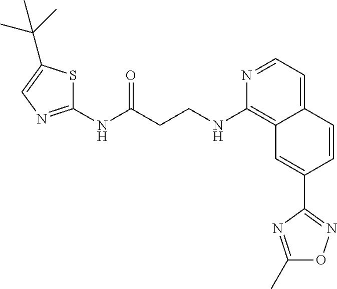 [SMILES: Cc1nc(-c2ccc3ccnc(NCCC(=O)Nc4ncc(C(C)(C)C)s4)c3c2)no1]

8 mg (27%). HPLC/MS m/z: 437.17, [M+H]⁺, Rt (Q): 2.62 min. ¹H NMR (600 MHz, DMSO-d₆) δ 11.94 (s, 1H), 8.91-8.84 (m, 1H), 8.14 (dd, J=8.5, 1.6 Hz, 1H), 7.99 (t, J=5.4 Hz, 1H), 7.98 (d, J=5.7 Hz, 1H), 7.85 (d, J=8.5 Hz, 1H), 7.12 (s, 1H), 6.97 (dd, J=5.9, 0.8 Hz, 1H), 3.79 (td, J=6.8, 5.2 Hz, 2H), 2.83 (t, J=6.8 Hz, 2H), 2.70 (s, 3H), 1.32 (s, 9H).

### Example 81: N-(5-(tert-butyl)-4-ethylthiazol-2-yl)-3-((7-(5-methyl-1,2,4-oxadiazol-3-yl)isoquinolin-1-yl)amino)propanamide

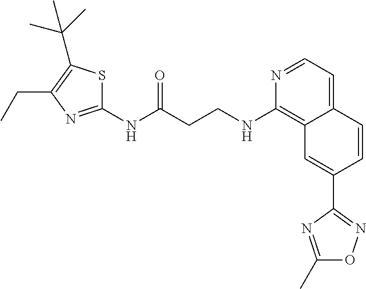 [SMILES: CCc1nc(NC(=O)CCNc2nccc3ccc(-c4noc(C)n4)cc23)sc1C(C)(C)C]

9.5 mg (30%) colorless solid. HPLC/MS m/z: 465.21, [M+H]⁺, Rt (U): 2.92 min. ¹H NMR (600 MHz, DMSO-d₆) δ 11.88 (s, 1H), 8.91-8.85 (m, 1H), 8.14 (dd, J=8.4, 1.5 Hz, 1H), 8.02-7.93 (m, 2H), 7.85 (d, J=8.5 Hz, 1H), 7.00-6.93 (m, 1H), 3.78 (td, J=6.9, 5.4 Hz, 2H), 2.80 (t, J=6.9 Hz, 2H), 2.70 (s, 3H), 2.64 (q, J=7.4 Hz, 2H), 1.36 (s, 9H), 1.15 (t, J=7.4 Hz, 3H).

### Example 82: N-(5-(tert-butyl)-1-methyl-1 H-pyrazol-3-yl)-3-((7-(5-methyl-1,2,4-oxadiazol-3-yl)isoquinolin-1-yl)amino)propanamide

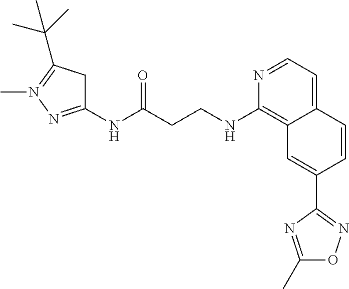

13 mg (45%) colorless solid. HPLC/MS m/z: 434.23, [M+H]⁺, Rt (U): 2.41 min. ¹H NMR (600 MHz, DMSO-d₆) δ 10.29 (s, 1H), 8.89 (s, 1H), 8.14 (dd, J=8.5, 1.5 Hz, 1H), 7.98 (d, J=5.7 Hz, 1H), 7.94 (s, 1H), 7.84 (d, J=8.5 Hz, 1H), 6.96 (d, J=5.8 Hz, 1H), 6.33 (s, 1H), 3.79-3.71 (m, 5H), 2.69 (d, J=11.8 Hz, 5H), 1.31 (s, 9H).

### Example 83: N-[5-(3-methoxyoxetan-3-yl)-4-methyl-thiazol-2-yl]-3-[[7-(5-methyl-1,2,4-oxadiazol-3-yl)-1-isoquinolyl]amino]propanamide

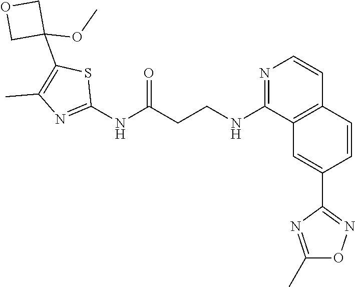 [SMILES: COC1(c2sc(NC(=O)CCNc3nccc4ccc(-c5noc(C)n5)cc34)nc2C)COC1]

Example 83.1: To a microwave vial was added 2-amino-4-methylthiazole (500.00 mg, 4.3794 mmol), p-toluene sulfonic acid (83.31 mg, 0.4379 mmol), 2,5-hexanedione (0.52 mL, 4.3794 mmol) and toluene (15.87 mL). The vial was capped and irradiated at 150° C. for 1 h. The mixture was diluted in MeOH and evaporated to dryness. Purification by silica gel column chromatography (eluent: 0-30% EtOAc in cyclohexane) to afford 2-(2,5-dimethylpyrrol-1-yl)-4-methyl-thiazole (565.7 mg, 67%, 2.9421 mmol) as a light-yellow oil. HPLC/MS m/z: 193.08, [M+H]⁺, Rt (T): 1.35 min.

Example 83.2.: 1.6 M n-Buli in hexane (0.98 mL, 1.5612 mmol) was added dropwise to a solution of 2-(2,5-dimethylpyrrol-1-yl)-4-methyl-thiazole (300.00 mg, 1.5602 mmol) in THF (11 mL) at −48° C. and the reaction stirred for 45 min (temperature warmed up a bit to −20° C. The temperature was then lowered to −48° C. and 3-oxetanone (75.00 mg, 1.0408 mmol) in a solution in THF (4 mL) was added and the reaction mixture stirred for 30 min. The temperature was left warming up gradually to rt (overnight). 1.65 mL of a saturated aqueous solution of NH₄Cl was added dropwise and the volatiles were evaporated nearly to completion. The crude was partioned between EtOAc (40 mL) and water (40 mL). After phase separation, the organic layer was washed with brine (40 mL), dried with MgSO₄, filtered and evaporated to dryness. Purification by silica gel column chromatography (eluent: 20-80% EtOAc in cyclohexane) afforded 3-[2-(2,5-dimethylpyrrol-1-yl)-4-methyl-thiazol-5-yl]oxetan-3-ol (220 mg, 80%, 0.8323 mmol) as a light yellow film. HPLC/MS m/z: 265.10, [M+H]⁺, Rt (T): 1.22 min.

Example 83.3.: NaH (83.23 mg, 2.0807 mmol) was added to a solution of 3-[2-(2,5-dimethylpyrrol-1-yl)-4-methyl-thiazol-5-yl]oxetan-3-ol (220.00 mg, 0.8323 mmol) in DMF (3.53 mL) at 0° C. The reaction mixture was stirred at 0° C. for 15 min followed by the addition of iodomethane (0.13 mL, 2.0807 mmol). The reaction was warmed up to rt and left stirring overnight. The reaction mixture was then partitioned between water (50 mL) and EtOAc (40 mL). After phase separation, the organic layer was washed with brine (50 mL), dried with MgSO₄, filtered, and evaporated to dryness to give 2-(2,5-dimethylpyrrol-1-yl)-5-(3-methoxyoxetan-3-yl)-4-methyl-thiazole (230.5 mg, 99%, 0.8280 mmol) as a light beige solid. This compound was taken forward with no further purification. HPLC/MS m/z: 279.12, [M+H]⁺, Rt (T): 1.35 min.

Example 83.4.: Concentrated HCl (0.07 mL, 2.1285 mmol) was added dropwise to a mixture of 2-(2,5-dimethylpyrrol-1-yl)-5-(3-methoxyoxetan-3-yl)-4-methyl-thiazole (79.00 mg, 0.2838 mmol) and MeOH (0.69 mL) in a vial. The vial was capped and irradiated in the microwave for 15 min at 90° C. The volatiles were carefully evaporated to dryness. The obtained crude was purified by SCX-II cartridge (2 g, 15 mL). The basic fraction was evaporated to dryness. Purification by silica gel column chromatography (eluent: 5% MeOH in DCM) to afford 5-(3-methoxyoxetan-3-yl)-4-methyl-thiazol-2-amine (11.2 mg, 20%, 0.056 mmol). HPLC/MS m/z: 171.06, [M+H]⁺, Rt (T): 0.44 min.

Example 83.5.: Using 5-(3-methoxyoxetan-3-yl)-4-methyl-thiazol-2-amine and the procedure for Example 45.7 afforded N-[5-(3-methoxyoxetan-3-yl)-4-methyl-thiazol-2-yl]-3-[[7-(5-methyl-1,2,4-oxadiazol-3-yl)-1-isoquinolyl]amino]propanamide (1.2 mg, 3%) as a colorless, amorphous solid. HPLC/MS m/z: 481.16 [M+H]⁺, Rt (T): 1.04 min. ¹H NMR (500 MHz, DMSO-d₆) δ 11.90 (brs, 1H), 8.91-8.86 (m, 1H), 8.15 (dd, J=8.5, 1.6 Hz, 1H), 8.02 (d, J=5.3 Hz, 1H), 7.97 (d, J=5.7 Hz, 1H), 7.85 (d, J=8.5 Hz, 1H), 6.69 (d, J=6.2 Hz, 1H), 4.78 (s, 4H), 3.80 (q, J=6.5 Hz, 2H), 3.05 (s, 3H), 2.85 (t, J=6.8 Hz, 2H), 2.70 (s, 3H), 2.13 (s, 3H).

### Example 84: 3-[[7-(5-methyl-1,2,4-oxadiazol-3-yl)-1-isoquinolyl]amino]-N-[4-methyl-5-(3-propoxyoxetan-3-yl)thiazol-2-yl]propenamide

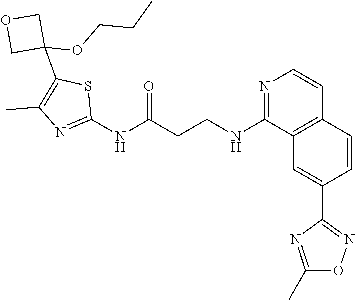 [SMILES: CCCOC1(c2sc(NC(=O)CCNc3nccc4ccc(-c5noc(C)n5)cc34)nc2C)COC1]

Example 84.1.: 2-(2,5-dimethylpyrrol-1-yl)-4-methyl-5-(3-propoxyoxetan-3-yl)thiazole was prepared using iodopropane in the procedure from Example 83. 2-(2,5-dimethylpyrrol-1-yl)-4-methyl-5-(3-propoxyoxetan-3-yl)thiazole (162.5 mg, 99%, 0.5303 mmol) as a light brown gum. HPLC/MS m/z: 307.15, [M+H]⁺, Rt (T): 1.52 min.

Example 84.2.: TFA (0.21 mL, 2.7413 mmol) was added dropwise to a solution of 2-(2,5-dimethylpyrrol-1-yl)-4-methyl-5-(3-propoxyoxetan-3-yl)thiazole (84.00 mg, 0.2741 mmol), 1-propanol (1.75 mL) and water (0.18 mL) in a microwave vial. The vial was capped and irradiated in the microwave for 2 h 5 min at 110 0° C. The reaction mixture was evaporated to dryness and then purified by SCX-II cartridge (2 g, 15 mL) using MeOH and 2 M solution of NH₃ in MeOH. Purification by silica gel column chromatography (eluent: 0-15% MeOH in DCM) afforded 4-methyl-5-(3-propoxyoxetan-3-yl)thiazol-2-amine (11.7 mg, 19%, 0.0512 mmol) as a brown, amorphous solid. HPLC/MS m/z: 229.10, [M+H]⁺, Rt (T): 0.73 min.

Example 84.3.: Using 4-methyl-5-(3-propoxyoxetan-3-yl)thiazol-2-amine amine and the procedure for Example 45.7 afforded 3-[[7-(5-methyl-1,2,4-oxadiazol-3-yl)-1-isoquinolyl]amino]-N-[4-methyl-5-(3-propoxyoxetan-3-yl)thiazol-2-yl]propanamide (17 mg, 71%) as a light beige solid. HPLC/MS m/z: 509.20 [M+H]⁺, Rt (P): 1.34 min. ¹H NMR (600 MHz, DMSO-d₆) δ 12.16 (s, 1H), 8.95-8.81 (m, 1H), 8.15 (dd, J=8.5, 1.6 Hz, 1H), 8.00 (t, J=5.5 Hz, 1H), 7.97 (d, J=5.7 Hz, 1H), 7.85 (d, J=8.5 Hz, 1H), 7.01-6.92 (m, 1H), 4.78 (s, 4H), 3.80 (q, J=6.7 Hz, 2H), 3.12 (t, J=6.5 Hz, 2H), 2.85 (t, J=6.8 Hz, 2H), 2.70 (s, 3H), 2.14 (s, 3H), 1.48 (sext, J=7.1 Hz, 2H), 0.83 (t, J=7.4 Hz, 3H).

### Example 85: N,4-dimethyl-2-[3-[[7-(5-methyl-1,2,4-oxadiazol-3-yl)-1-isoquinolyl]amino]propanoylamino]-N-propyl-thiazole-5-carboxamide

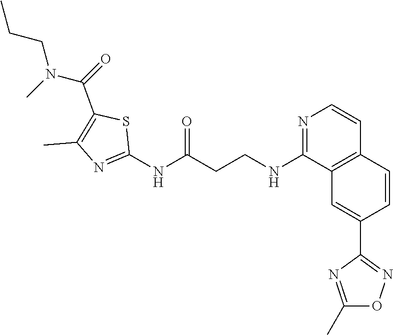 [SMILES: CCCN(C)C(=O)c1sc(NC(=O)CCNc2nccc3ccc(-c4noc(C)n4)cc23)nc1C]

Example 85.1.: 2-Amino-4-methyl-thiazole-5-carboxylic acid (100.00 mg, 0.6322 mmol), DIPEA (0.44 mL, 2.5288 mmol), HATU (223.09 mg, 0.9483 mmol) were suspended in dry DMF (3.61 mL) under nitrogen before N-methylpropan-1-amine (0.10 mL, 0.9483 mmol) was added. The mixture stirred at rt for 22 h. The reaction mixture was partitioned between EtOAc (50 mL) and water (40 mL). The organic was retained and washed with aqueous saturated bicarbonate (50 mL) and brine (50 mL), dried over MgSO₄, concentrated to dryness in vaccum to afford 2-amino-N,4-dimethyl-N-propyl-thiazole-5-carboxamide (44 mg, 33%, 0.2063 mmol) as a yellow solid. HPLC/MS m/z: 214.10, [M+H]⁺, Rt (T): 0.63 min.

Example 85.2. Using 2-amino-N,4-dimethyl-N-propyl-thiazole-5-carboxamide and the procedure for Example 45.7 afforded N,4-dimethyl-2-[3-[[7-(5-methyl-1,2,4-oxadiazol-3-yl)-1-isoquinolyl]amino]propanoylamino]-N-propyl-thiazole-5-carboxamide (24.6 mg, 50%, 0.0498 mmol) as a clear film. HPLC/MS m/z: 494.20 [M+H]⁺, Rt (T): 1.07 min. ¹H NMR (600 MHz, MeOD-d₄) δ 8.87-8.77 (m, 1H), 8.20 (dd, J=8.5, 1.6 Hz, 1H), 7.95 (dd, J=6.0, 1.7 Hz, 1H), 7.86-7.76 (m, 1H), 6.99 (dt, J=5.9, 1.4 Hz, 1H), 3.96 (t, J=6.7 Hz, 2H), 3.50-3.41 (m, 2H), 3.06 (s, 3H), 2.96 (t, J=6.7 Hz, 2H), 2.69 (s, 3H), 2.28 (s, 3H), 1.66 (brd, J=8.5 Hz, 2H), 0.90 (brs, 3H).

### Example 86: N-[5-(cyclohexen-1-yl)thiazol-2-yl]-3-[[7-(5-methyl-1,2,4-oxadiazol-3-yl)-1-isoquinolyl]amino]propenamide

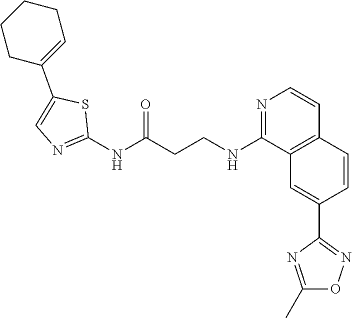 [SMILES: Cc1nc(-c2ccc3ccnc(NCCC(=O)Nc4ncc(C5=CCCCC5)s4)c3c2)no1]

To 1-(2-amino-1,3-thiazol-5-yl)cyclohexan-1-ol (15.39 mg, 0.0738 mmol), 3-(ethyliminomethyleneamino)-N,N-dimethyl-propan-1-amine hydrochloride (25.71 mg, 0.1341 mmol) and HOBt (18.12 mg, 0.1341 mmol) under nitrogen atmosphere was added 3-[[7-(5-methyl-1,2,4-oxadiazol-3-yl)-1-isoquinolyl]amino]propanoic acid (20.00 mg, 0.0670 mmol) in DMF (0.34 mL) and the reaction mixture heated at 70° C. for 20 h. Purified by reverse phase column chromatography (eluent: 10-100% MeOH in water (+0.1% formic acid modifier in both)) to afford N-[5-(cyclohexen-1-yl)thiazol-2-yl]-3-[[7-(5-methyl-1,2,4-oxadiazol-3-yl)-1-isoquinolyl]amino]-propanamide (8.2 mg, 27%, 0.0178 mmol) as a a colorless solid. HPLC/MS m/z: 461.18 [M+H]⁺, Rt (T): 1.36 min. ¹H NMR (600 MHz, DMSO-d₆) δ 12.08 (s, 1H), 8.88 (d, J=1.6 Hz, 1H), 8.14 (dd, J=8.5, 1.5 Hz, 1H), 8.01 (t, J=5.4 Hz, 1H), 7.97 (d, J=5.7 Hz, 1H), 7.85 (d, J=8.5 Hz, 1H), 7.30 (s, 1H), 6.97 (d, J=5.7 Hz, 1H), 5.97 (tt, J=4.0, 1.6 Hz, 1H), 3.79 (q, J=6.4 Hz, 2H), 2.85 (t, J=6.8 Hz, 2H), 2.70 (s, 3H), 2.33 (ddt, J=6.4, 4.4, 2.0 Hz, 2H), 2.21-2.11 (m, 2H), 1.72-1.65 (m, 2H), 1.62-1.54 (m, 2H).

### Example 87: N-[5-(1-methoxycyclohexyl)thiazol-2-yl]-3-[[7-(5-methyl-1,2,4-oxadiazol-3-yl)-1-isoquinolyl]amino]propenamide

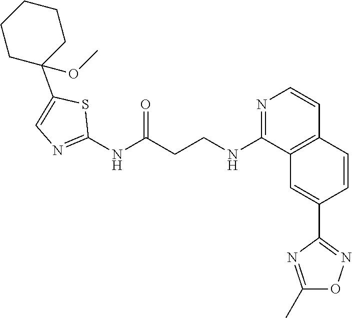 [SMILES: COC1(c2cnc(NC(=O)CCNc3nccc4ccc(-c5noc(C)n5)cc34)s2)CCCCC1]

To 3-[[7-(5-methyl-1,2,4-oxadiazol-3-yl)-1-isoquinolyl]amino]propanoic acid (20.00 mg, 0.0670 mmol) was added 1-(2-amino-1,3-thiazol-5-yl)cyclohexan-1-ol (27.99 mg, 0.1341 mmol) and PyBrop (75.01 mg, 0.1609 mmol) followed by dry DMF (0.34 mL) and DIPEA (42.04 uL, 0.2414 mmol). The resulting solution was stirred at rt for 19 h. Purified by reverse phase column chromatography (eluent: 10-100% MeOH in water (+0.1% formic acid in both). Further purification by ion exchange SCX-II column chromatography, washing with MeOH before eluting with 2M NH₃ in MeOH afforded a mixture of N-[5-(1-hydroxycyclohexyl)thiazol-2-yl]-3-[[7-(5-methyl-1,2,4-oxadiazol-3-yl)-1-isoquinolyl]amino]propanamide and N-[5-(1-methoxycyclohexyl)thiazol-2-yl]-3-[[7-(5-methyl-1,2,4-oxadiazol-3-yl)-1-isoquinolyl]amino]propanamide. Prep TLC (500 microns) using 5% MeOH in DCM as eluent afforded N-[5-(1-methoxycyclohexyl)thiazol-2-yl]-3-[[7-(5-methyl-1,2,4-oxadiazol-3-yl)-1-isoquinolyl]amino]propanamide (3 mg, 8%) as a colorless, amorphous solid. HPLC/MS m/z: 493.21 [M+H]⁺, Rt (P): 1.34 min. ¹H NMR (600 MHz, DMSO-d₆) δ 12.07 (s, 1H), 8.89 (d, J=1.6 Hz, 1H), 8.15 (dd, J=8.5, 1.5 Hz, 1H), 8.01 (t, J=5.4 Hz, 1H), 7.97 (d, J=5.7 Hz, 1H), 7.85 (d, J=8.5 Hz, 1H), 7.29 (s, 1H), 6.97 (d, J=5.7 Hz, 1H), 3.80 (q, J=6.4 Hz, 2H), 2.93 (s, 3H), 2.85 (t, J=6.8 Hz, 2H), 2.70 (s, 3H), 1.96 (d, J=13.4 Hz, 2H), 1.82-1.68 (m, 2H), 1.61-1.42 (m, 3H), 1.30-1.21 (m, 3H).

### Example 88: Ethyl 4-isopropyl-2-[3-[[3-(5-methyl-1,2,4-oxadiazol-3-yl)benzoyl]amino]propanoylamino]thiazole-5-carboxylate

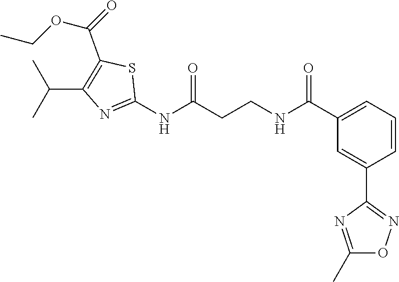 [SMILES: CCOC(=O)c1sc(NC(=O)CCNC(=O)c2cccc(-c3noc(C)n3)c2)nc1C(C)C]

To a stirred solution of 3-[[3-(5-methyl-1,2,4-oxadiazol-3-yl)benzoyl]amino]-propanoic acid [Example 63.3](35 mg, 0.13 mmol), ethyl 2-amino-4-isopropyl-thiazole-5-carboxylate (25 mg, 0.12 mmol) and DIPEA (0.030 mL, 0.17 mmol) in DMF (0.98 mL) was added HATU (57 mg, 0.15 mmol) at rt and the resulting mixture stirred for 17 h. The reaction mixture was diluted with saturated aqueous sodium bicarbonate (10 mL) and extracted with ethyl acetate (10 mL). The organic layer was washed with brine (10 mL), dried over anhydrous sodium sulfate, filtered, and concentrated under reduced pressure. The residue was purified by reverse phase column chromatography (eluent: methanol/water, 0.1% formic acid modifier=20 to 100% gradient) to afford 0.031 g (57%) as an off-white solid. HPLC/MS m/z: 472.16 [M+H]⁺, Rt (R): 1.42 min. ¹H NMR (500 MHz, DMSO-d₆) δ 12.59 (s, 1H), 8.85 (t, J=5.5 Hz, 1H), 8.46 (t, J=1.7 Hz, 1H), 8.13 (dt, J=7.8, 1.3 Hz, 1H), 8.02 (dt, J=7.9, 1.3 Hz, 1H), 7.65 (t, J=7.8 Hz, 1H), 4.24 (q, J=7.1 Hz, 2H), 3.89 (quint, J=6.9 Hz, 1H), 3.60 (q, J=6.6 Hz, 2H), 2.78 (t, J=6.8 Hz, 2H), 2.68 (s, 3H), 1.27 (t, J=7.1 Hz, 3H), 1.18 (d, J=6.8 Hz, 6H).

The following examples were prepared in an analogous procedure:

### Example 89: tert-Butyl-4-methyl-2-(3-(3-(1-methyl-1 H-pyrazol-4-yl)benzamido)propanamido)thiazole-5-carboxylate

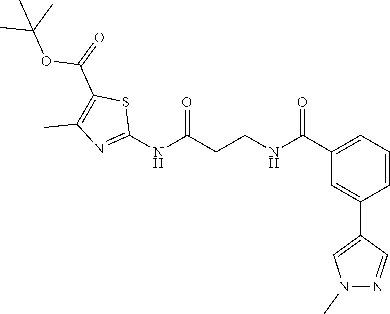 [SMILES: Cc1nc(NC(=O)CCNC(=O)c2cccc(-c3cnn(C)c3)c2)sc1C(=O)OC(C)(C)C]

Using 3-(1-methylpyrazol-4-yl)benzoic acid and a procedure from Example 63.3 afforded 3-[[3-(1-methylpyrazol-4-yl)benzoyl]amino]propanoic acid which was used in the procedure above to afford tert-butyl 4-methyl-2-(3-(3-(1-methyl-1 H-pyrazol-4-yl)benzamido)propanamido)thiazole-5-carboxylate (25 mg, 26%, 0.0532 mmol) as a pale-yellow powder. HPLC/MS m/z: 470.2, [M+H]⁺, Rt (Z): 2.72 min. ¹H NMR (600 MHz, DMSO-d₆) δ 12.43 (s, 1H), 8.62 (t, J=5.6 Hz, 1H), 8.16 (d, J=0.8 Hz, 1H), 7.99 (t, J=1.8 Hz, 1H), 7.88 (d, J=0.9 Hz, 1H), 7.69 (ddd, J=7.7, 1.8, 1.1 Hz, 1H), 7.62 (dt, J=7.8, 1.3 Hz, 1H), 7.43 (t, J=7.7 Hz, 1H), 3.87 (s, 3H), 3.63-3.55 (m, 2H), 2.77 (t, J=6.8 Hz, 2H), 2.50 (s, 3H), 1.50 (s, 9H).

### Example 90: Methyl 4-ethyl-2-[3-[[3-(5-methyl-1,2,4-oxadiazol-3-yl)benzoyl]amino]propanoylamino]thiazole-5-carboxylate

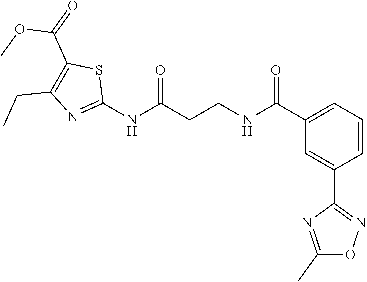 [SMILES: CCc1nc(NC(=O)CCNC(=O)c2cccc(-c3noc(C)n3)c2)sc1C(=O)OC]

To a stirred solution of 3-[[3-(5-methyl-1,2,4-oxadiazol-3-yl)benzoyl]amino]-propanoic acid [Example 63.3](35 mg, 0.13 mmol), methyl 2-amino-4-ethyl-thiazole-5-carboxylate (23 mg, 0.12 mmol) and DIPEA (0.030 mL, 0.17 mmol) in DMF (0.98 mL) was added HATU (57 mg, 0.15 mmol) at rt and the resulting mixture stirred for 17 h. The reaction mixture was diluted with saturated aqueous sodium bicarbonate (10 mL) and extracted with ethyl acetate (10 mL). The organic layer was washed with brine (10 mL), dried over anhydrous sodium sulfate, filtered, and concentrated under reduced pressure. The residue was purified by reverse phase column chromatography (eluent: methanol/water, 0.1% formic acid modifier=20 to 100% gradient) to afford 0.035 g (64%) as an off-white solid. HPLC/MS m/z: 444.13 [M+H]⁺, Rt (R): 1.30 min. ¹H NMR (500 MHz, DMSO-d₆) δ 12.58 (s, 1H), 8.86 (t, J=5.5 Hz, 1H), 8.46 (t, J=1.7 Hz, 1H), 8.13 (dd, J=7.8, 1.7 Hz, 1H), 8.02 (dd, J=7.8, 1.7 Hz, 1H), 7.65 (t, J=7.8 Hz, 1H), 3.77 (s, 3H), 3.64-3.56 (m, 2H), 2.98 (q, J=7.5 Hz, 2H), 2.78 (t, J=6.8 Hz, 2H), 2.68 (s, 3H), 1.17 (t, J=7.5 Hz, 3H).

### Example 91: Ethyl 2-[3-[[3-(5-methyl-1,2,4-oxadiazol-3-yl)benzoyl]amino]propanoylamino]-4-(trifluoromethyl)thiazole-5-carboxylate

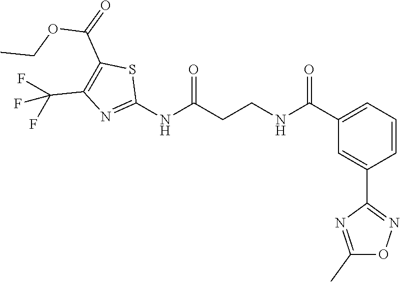 [SMILES: CCOC(=O)c1sc(NC(=O)CCNC(=O)c2cccc(-c3noc(C)n3)c2)nc1C(F)(F)F]

To a stirred solution of 3-[[3-(5-methyl-1,2,4-oxadiazol-3-yl)benzoyl]amino]-propanoic acid [Example 63.3](35 mg, 0.13 mmol), ethyl 2-amino-4-(trifluoromethyl)thiazole-5-carboxylate (28 mg, 0.12 mmol) and DIPEA (0.030 mL, 0.17 mmol) in DMF (0.98 mL) was added HATU (57 mg, 0.15 mmol) at rt and the resulting mixture stirred for 17 h. The reaction mixture was diluted with saturated aqueous sodium bicarbonate (10 mL) and extracted with ethyl acetate (10 mL). The organic layer was washed with brine (10 mL), dried over anhydrous sodium sulfate, filtered, and concentrated under reduced pressure. The residue was purified by reverse phase column chromatography (eluent: methanol/water, 0.1% formic acid modifier=20 to 100% gradient) to afford 0.014 g (24%) as an off-white solid. HPLC/MS m/z: 498.11 [M+H]⁺, Rt (R): 1.37 min. ¹H NMR (500 MHz, DMSO-d₆) δ 13.10 (s, 1H), 8.86 (t, J=5.6 Hz, 1H), 8.45 (t, J=1.7 Hz, 1H), 8.13 (dt, J=7.7, 1.4 Hz, 1H), 8.02 (dt, J=7.9, 1.4 Hz, 1H), 7.65 (t, J=7.8 Hz, 1H), 4.31 (q, J=7.1 Hz, 2H), 3.62 (q, J=6.5 Hz, 2H), 2.82 (t, J=6.8 Hz, 2H), 2.68 (s, 3H), 1.29 (t, J=7.1 Hz, 3H).

### Example 92: Ethyl 4-ethyl-2-[3-[[3-(5-methyl-1,2,4-oxadiazol-3-yl)benzoyl]amino]propanoylamino]thiazole-5-carboxylate

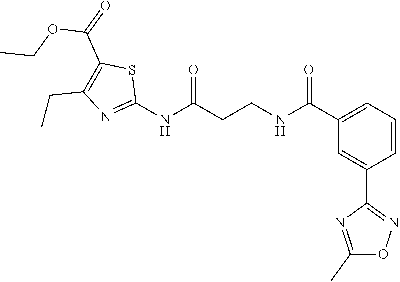 [SMILES: CCOC(=O)c1sc(NC(=O)CCNC(=O)c2cccc(-c3noc(C)n3)c2)nc1CC]

To a stirred solution of 3-[[3-(5-methyl-1,2,4-oxadiazol-3-yl)benzoyl]amino]-propanoic acid [0](35 mg, 0.13 mmol), ethyl 2-amino-4-ethyl-thiazole-5-carboxylate (23 mg, 0.12 mmol) and DIPEA (0.030 mL, 0.17 mmol) in DMF (0.98 mL) was added HATU (57 mg, 0.15 mmol) at rt and the resulting mixture stirred for 23 h. The reaction mixture was diluted with saturated aqueous sodium bicarbonate (10 mL) and extracted with ethyl acetate (10 mL). The organic layer was washed with brine (10 mL), dried over anhydrous sodium sulfate, filtered, and concentrated under reduced pressure. The residue was purified by reverse phase column chromatography (eluent: methanol/water, 0.1% formic acid modifier=40 to 100% gradient) to afford 0.023 g (43%) as an off-white solid. HPLC/MS m/z: 458.15 [M+H]⁺, Rt (R): 1.36 min. ¹H NMR (500 MHz, DMSO-d₆) δ 12.57 (s, 1H), 8.86 (t, J=5.5 Hz, 1H), 8.46 (t, J=1.7 Hz, 1H), 8.13 (dt, J=7.7, 1.4 Hz, 1H), 8.02 (dt, J=7.9, 1.3 Hz, 1H), 7.65 (t, J=7.8 Hz, 1H), 4.23 (q, J=7.1 Hz, 2H), 3.60 (q, J=6.5 Hz, 2H), 2.98 (q, J=7.5 Hz, 2H), 2.78 (t, J=6.8 Hz, 2H), 2.68 (s, 3H), 1.27 (t, J=7.1 Hz, 3H), 1.17 (t, J=7.5 Hz, 3H).

### Example 93: N-[3-[[5-[(E)-2-cyclopentylvinyl]-4-methyl-thiazol-2-yl]amino]-3-oxo-propyl]-3-(5-methyl-1,2,4-oxadiazol-3-yl)benzamide

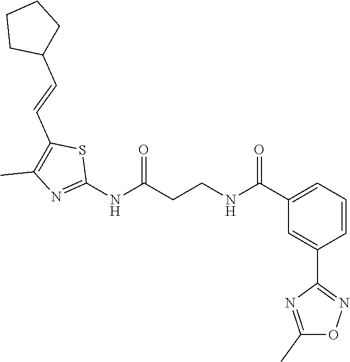 [SMILES: Cc1nc(-c2cccc(C(=O)NCCC(=O)Nc3nc(C)c(/C=C/C4CCCC4)s3)c2)no1]

Example 93.1.: To a stirred solution of 3-[[3-(5-methyl-1,2,4-oxadiazol-3-yl)benzoyl]amino]propanoic acid [Example 63.3](600 mg, 2.18 mmol), 5-bromo-4-methyl-thiazol-2-amine (421 mg, 2.18 mmol) and DIPEA (0.570 mL, 3.27 mmol) in DMF (16.8 mL) was added HATU (1078 mg, 2.83 mmol) at rt and the resulting mixture stirred for 20 h. The reaction mixture was diluted with saturated aqueous sodium bicarbonate (150 mL) and extracted with ethyl acetate (3×50 mL). The combined organic layers were washed with brine (80 mL), dried over anhydrous sodium sulfate, filtered, and concentrated under reduced pressure. The residue was purified by column chromatography (eluent: ethyl acetate/cyclohexane=50 to 100% gradient) to afford 0.506 g (52%) of N-[3-[(5-bromo-4-methyl-thiazol-2-yl)amino]-3-oxo-propyl]-3-(5-methyl-1,2,4-oxadiazol-3-yl)benzamide. HPLC/MS m/z: 450/452 Br split [M+H]⁺, Rt (R): 1.35 min. ¹H NMR (500 MHz, DMSO-d₆) δ 12.36 (s, 1H), 8.87 (t, J=5.5 Hz, 1H), 8.46 (t, J=1.7 Hz, 1H), 8.12 (dt, J=7.7, 1.4 Hz, 1H), 8.02 (dt, J=8.0, 1.4 Hz, 1H), 7.65 (t, J=7.8 Hz, 1H), 3.58 (q, J=6.5 Hz, 2H), 2.75 (t, J=6.8 Hz, 2H), 2.69 (s, 3H), 2.21 (s, 3H).

Example 93.2.: Nitrogen gas was bubbled through a mixture of N-[3-[(5-bromo-4-methyl-thiazol-2-yl)amino]-3-oxo-propyl]-3-(5-methyl-1,2,4-oxadiazol-3-yl)benzamide (50 mg, 0.11 mmol) and [(E)-2-cyclopentylvinyl]boronic acid (23 mg, 0.17 mmol) in 1,4-dioxane (1.11 mL) and 2 M aqueous sodium carbonate (0.22 mL, 0.44 mmol) for 5 min. Palladium tetrakis(triphenylphosphine) (13 mg, 0.011 mmol) was added and the mixture heated at 110° C. in a sealed vial for 30 min. The reaction mixture was diluted with brine (10 mL) and extracted with ethyl acetate (2×10 mL). The combined organic layers were dried over anhydrous sodium sulfate, filtered, and concentrated under reduced pressure. The residue was purified by column chromatography (eluent: ethyl acetate/cyclohexane=50 to 100% gradient) and reverse phase column chromatography (eluent: methanol/water, 0.1% formic acid modifier=20 to 100% gradient) to afford 0.023 g (44%) as an off-white solid. HPLC/MS m/z: 466.19 [M+H]⁺, Rt (P): 1.67 min. ¹H NMR (500 MHz, DMSO-d₆) δ 12.05 (s, 1H), 8.85 (t, J=5.5 Hz, 1H), 8.46 (t, J=1.7 Hz, 1H), 8.13 (dt, J=7.7, 1.4 Hz, 1H), 8.03 (dt, J=7.9, 1.3 Hz, 1H), 7.65 (t, J=7.8 Hz, 1H), 6.48 (dd, J=15.6, 1.1 Hz, 1H), 5.75 (dd, J=15.6, 7.9 Hz, 1H), 3.58 (q, J=6.6 Hz, 2H), 2.73 (t, J=6.9 Hz, 2H), 2.68 (s, 3H), 2.61-2.51 (m, 1H), 2.21 (s, 3H), 1.84-1.73 (m, 2H), 1.71-1.47 (m, 4H), 1.39-1.28 (m, 2H).

### Example 94: N-[3-[[5-[(E)-3-methoxyprop-1-enyl]-4-methyl-thiazol-2-yl]amino]-3-oxo-propyl]-3-(5-methyl-1,2,4-oxadiazol-3-yl)benzamide

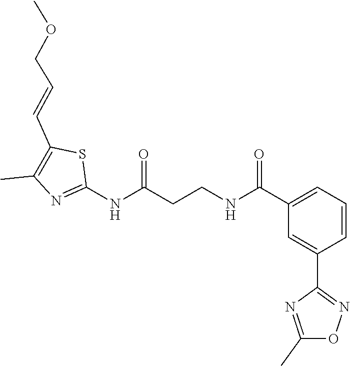 [SMILES: COC/C=C/c1sc(NC(=O)CCNC(=O)c2cccc(-c3noc(C)n3)c2)nc1C]

Nitrogen gas was bubbled through a mixture of N-[3-[(5-bromo-4-methyl-thiazol-2-yl)amino]-3-oxo-propyl]-3-(5-methyl-1,2,4-oxadiazol-3-yl)benzamide (50 mg, 0.11 mmol) [Example 93.1] and 2-[(E)-3-methoxyprop-1-enyl]-4,4,5,5-tetramethyl-1,3,2-dioxaborolane (33 mg, 0.17 mmol) in 1,4-dioxane (1.11 mL) and 2 M aqueous sodium carbonate (0.22 mL, 0.44 mmol) for 5 min. Palladium tetrakis(triphenyl-phosphine) (13 mg, 0.011 mmol) was added and the mixture heated at 110° C. in a sealed vial for 30 min. The reaction mixture was diluted with brine (10 mL) and extracted with ethyl acetate (2×10 mL). The combined organic layers were dried over anhydrous sodium sulfate, filtered, and concentrated under reduced pressure. The residue was purified by column chromatography (eluent: ethyl acetate/cyclohexane=50 to 100% gradient), reverse phase column chromatography (eluent: methanol/water, 0.1% formic acid modifier=20 to 100% gradient) and column chromatography (eluent: methanol/dichloromethane=0 to 8% gradient) to afford 0.011 g (22%) as a pale-yellow gum. HPLC/MS m/z: 442.15 [M+H]⁺, Rt (R): 1.29 min. ¹H NMR (500 MHz, MeOD-d₄) δ 8.49 (td, J=1.7, 0.5 Hz, 1H), 8.18 (ddd, J=7.7, 1.7, 1.2 Hz, 1H), 7.96 (ddd, J=7.8, 1.8, 1.2 Hz, 1H), 7.61 (td, J=7.8, 0.6 Hz, 1H), 6.74 (dt, J=15.7, 1.4 Hz, 1H), 5.89 (dt, J=15.7, 6.1 Hz, 1H), 4.05 (dd, J=6.1, 1.4 Hz, 2H), 3.76 (t, J=6.7 Hz, 2H), 3.36 (s, 3H), 2.83 (t, J=6.7 Hz, 2H), 2.66 (s, 3H), 2.27 (s, 3H).

### Example 95: 3-(5-Methyl-1,2,4-oxadiazol-3-yl)-N-[3-[[4-methyl-5-(1-phenylvinyl)thiazol-2-yl]amino]-3-oxo-propyl]benzamide

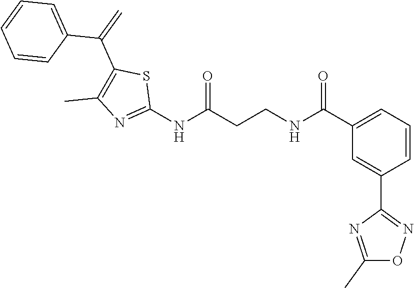 [SMILES: C=C(c1ccccc1)c1sc(NC(=O)CCNC(=O)c2cccc(-c3noc(C)n3)c2)nc1C]

Nitrogen gas was bubbled through a mixture of N-[3-[(5-bromo-4-methyl-thiazol-2-yl)amino]-3-oxo-propyl]-3-(5-methyl-1,2,4-oxadiazol-3-yl)benzamide (50 mg, 0.11 mmol) [Example 93.1], 4,4,5,5-tetramethyl-2-(1-phenylvinyl)-1,3,2-dioxaborolane (38 mg, 0.17 mmol) in 1,4-dioxane (1.11 mL) and 2 M aqueous sodium carbonate (0.22 mL, 0.44 mmol) for 5 min. Palladium tetrakis(triphenylphosphine) (13 mg, 0.011 mmol) was added and the mixture heated at 110° C. in a sealed vial for 30 min. The reaction mixture was diluted with brine (10 mL) and extracted with ethyl acetate (2×10 mL). The combined organic layers were dried over anhydrous sodium sulfate, filtered, and concentrated under reduced pressure. The residue was purified by column chromatography (eluent: ethyl acetate/cyclohexane=50 to 100% gradient) and reverse phase column chromatography (eluent: methanol/water, 0.1% formic acid modifier=20 to 100% gradient) to afford 0.018 g (34%) as a pale-yellow gum. HPLC/MS m/z: 474.16 [M+H]⁺, Rt (R): 1.48 min. ¹H NMR (500 MHz, MeOD-d₄) δ 8.49 (td, J=1.8, 0.6 Hz, 1H), 8.18 (ddd, J=7.7, 1.7, 1.1 Hz, 1H), 7.96 (ddd, J=7.8, 1.9, 1.2 Hz, 1H), 7.60 (td, J=7.8, 0.6 Hz, 1H), 7.37-7.29 (m, 5H), 5.64 (d, J=1.1 Hz, 1H), 5.37 (d, J=1.2 Hz, 1H), 3.76 (t, J=6.7 Hz, 2H), 2.84 (t, J=6.7 Hz, 2H), 2.65 (s, 3H), 2.02 (s, 3H).

### Example 96: 3-[[7-(5-methyl-1,2,4-oxadiazol-3-yl)-1-isoquinolyl]amino]-N-[5-(1,1,2,2,2-pentafluoroethyl)thiazol-2-yl]cyclobutanecarboxamide

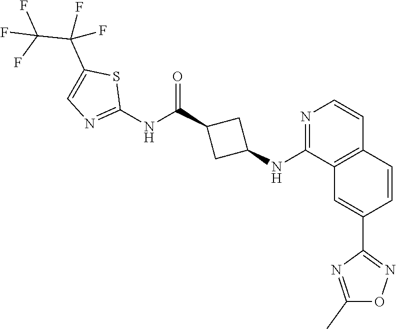 [SMILES: Cc1nc(-c2ccc3ccnc(N[C@H]4C[C@@H](C(=O)Nc5ncc(C(F)(F)C(F)(F)F)s5)C4)c3c2)no1]

Example 96.1.: Isoquinoline-7-carbonitrile (4.00 g, 25.9 mmol), triethylamine (7.23 mL, 51.9 mmol) and [bmim]OAc (26 mL) were mixed and heated to 80° C. Hydroxylamine hydrochloride (3.61 g, 51.9 mmol) was added. The reaction mixture was continued to stir at 80° C. for 1.5 h. The reaction mixture was cooled to rt and mixed thoroughly with EtOAc (250 mL). The emulsion was then mixed with water (750 mL). The layers were separated, and the aqueous layer was further extracted with EtOAc (5×100 mL). The combined organic layer was washed with saturated NaCl (3×100 mL), dried over anhydrous MgSO₄, filtered and concentrated under reduced pressure to yield 3.70 g (76%) of N′-hydroxyisoquinoline-7-carboxamidine as an off-white solid. HPLC/MS m/z: 188.0829 [M+H]⁺, Rt (S): 0.37 min.

Example 96.2.: Four individual 20 mL microwave vials were each charged with a quarter of the following: N′-hydroxyisoquinoline-7-carboxamidine (3.67 g, 19.6 mmol) was mixed with acetonitrile (40 mL) and acetic anhydride (2.2 mL, 23.5 mmol) under an argon atmosphere. Each portion of the reaction mixture was heated at 180° C. under microwave irradiation for 10 min. The combined reaction mixture was evaporated onto silica gel and purified by flash chromatography (20-80% EtOAc in cyclohexane) to yield 3.59 g (87%) of 3-(7-isoquinolyl)-5-methyl-1,2,4-oxadiazole as an off-white solid. HPLC/MS m/z: 212.1 [M+H]⁺, Rt (R): 0.88 min.

Example 96.3.: 3-(7-isoquinolyl)-5-methyl-1,2,4-oxadiazole (3.59 g, 17.0 mmol) was suspended in anhydrous chloroform (57 mL) under an argon atmosphere and cooled in an ice bath. 3-Chloroperoxybenzoic acid (4.57 g, 20.4 mmol) was added. The stirred reaction mixture was allowed to warm to ambient temperature and continued to stir overnight. Potassium carbonate (9.40 g, 68.0 mmol) was added. The mixture was stirred at rt for 4 h before filtering through a pad of anhydrous MgSO₄. The filtrate was concentrated under reduced pressure to yield 5-methyl-3-(2-oxidoisoquinolin-2-ium-7-yl)-1,2,4-oxadiazole (3.12 g, 81%) as an off-white powder. HPLC/MS m/z: 228.0713 [M+H]⁺, Rt (X): 1.73 min.

Example 96.4.: 5-Methyl-3-(2-oxidoisoquinolin-2-ium-7-yl)-1,2,4-oxadiazole [Example 96](900.00 mg, 3.9609 mmol) and methyl cis-3-amino-cyclobutanecarboxylate hydrochloride (820.01 mg, 4.9511 mmol) were mixed in DCM (7.92 mL) at rt under argon atmosphere. DIPEA (3.28 mL, 18.814 mmol) and PyBrop (2400.46 mg, 5.1492 mmol) were added, and the reaction mixture was stirred at rt for 3 d. Volatiles were removed in vacuo and the crude was purified by silica gel column chromatography (eluent: 20-60% EtOAc in cyclohexane) to give methyl 3-[[7-(5-methyl-1,2,4-oxadiazol-3-yl)-1-isoquinolyl]amino]cyclobutane-carboxylate (1090.6 mg, 81%, 3.2232 mmol) as an off-white solid. HPLC/MS m/z: 339.15, [M+H]⁺, Rt (T): 1.05 min.

Example 96.5.: Methyl 3-[[7-(5-methyl-1,2,4-oxadiazol-3-yl)-1-isoquinolyl]amino]-cyclobutanecarboxylate (1000.00 mg, 2.9554 mmol), THF (11.82 mL), MeOH (5.91 mL) and water (11.82 mL) were mixed at ambient temperature. LiOH monohydrate (248.02 mg, 5.9109 mmol) was added, and the reaction mixture was stirred for 1 h. The reaction mixture was concentrated in vacuo. Purified by reverse phase column chromatography (eluent: 5-30% MeOH in water). The fractions were combined, concentrated in vacuo to give 3-[[7-(5-methyl-1,2,4-oxadiazol-3-yl)-1-isoquinolyl]-amino]cyclobutanecarboxylic acid (776.9 mg, 81%, 2.3954 mmol) as a fine, colorless solid. HPLC/MS m/z: 325.13, [M+H]⁺, Rt (T): 0.97 min.

Example 96.6.: 3-[[7-(5-methyl-1,2,4-oxadiazol-3-yl)-1-isoquinolyl]amino]-cyclobutanecarboxylic acid formate (40.00 mg, 0.1080 mmol) and bis(tetramethylene)fluoroformamidinium (68.30 mg, 0.2160 mmol) were mixed in DCM (0.43 mL) at rt under nitrogen atmosphere for 10 min. DIPEA (0.09 mL, 0.4860 mmol) was added and the reaction mixture was stirred for 30 min. 5-(pentafluoroethyl)-1,3-thiazol-2-amine (47.12 mg, 0.2160 mmol) was added. The vial was capped, and the reaction was heated to 80° C. for 1 h in the microwave. Volatiles were removed in vacuo and the crude reaction mixture was directly purified by flash chromatography (5-70% MeOH in water [0.1% FA]). Fractions containing product were filtered through a 2 g SCX-2 column. The compound was released with 2 N NH₃ in MeOH and the solvent was removed in vacuo which afforded the desired product 3-[[7-(5-methyl-1,2,4-oxadiazol-3-yl)-1-isoquinolyl]amino]-N-[5-(1,1,2,2,2-pentafluoroethyl)thiazol-2-yl]cyclobutane-carboxamide (12.3 mg, 21%, 0.0231 mmol) as a fine, yellow solid. HPLC/MS m/z 525.1133 [M+H]⁺, Rt (U): 3.05 min. ¹H NMR (600 MHz, DMSO-d₆) δ 12.79 (s, 1H), 8.74-9.27 (m, 1H), 8.15 (dd, J=1.6, 8.5 Hz, 1H), 8.07-8.12 (m, 2H), 7.96 (d, J=5.7 Hz, 1H), 7.85 (d, J=8.5 Hz, 1H), 6.94-7.00 (m, 1H), 4.72 (ddt, J=7.4, 9.3, 16.7 Hz, 1H), 3.17 (d, J=5.3 Hz, 2H), 2.71 (s, 3H), 2.63 (qd, J=2.6, 7.8 Hz, 2H), 2.45 (qd, J=2.6, 9.3 Hz, 2H).

### Example 97: N-(5-bromo-4-methyl-thiazol-2-yl)-3-[[7-(5-methyl-1,2,4-oxadiazol-3-yl)-1-isoquinolyl]amino]cyclobutanecarboxamide

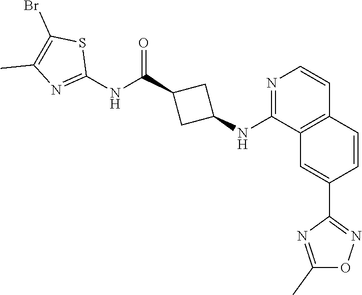 [SMILES: Cc1nc(-c2ccc3ccnc(N[C@H]4C[C@@H](C(=O)Nc5nc(C)c(Br)s5)C4)c3c2)no1]

3-[[7-(5-methyl-1,2,4-oxadiazol-3-yl)-1-isoquinolyl]amino]cyclobutanecarboxylic acid [Example 96](265.00 mg, 0.8171 mmol) and bis(tetramethylene)fluoro-formamidinium (335.86 mg, 1.0622 mmol) were mixed in DCM (3.27 mL) at rt under nitrogen atmosphere for 10 min. DIPEA (0.64 mL, 3.6768 mmol) was added and the reaction mixture was stirred for 30 min. 5-Bromo-4-methyl-thiazol-2-amine (205.07 mg, 1.0622 mmol) was added. The vial was capped, and the reaction was heated to 80° C. for 2 h in the microwave. Volatiles were removed in vacuo and the crude reaction mixture was directly purified by flash chromatography (5-70% MeOH in water [0.1% FA]). Fractions containing product were filtered through a 2 g SCX-2 column. The compound was released with 2 N NH₃ in MeOH and the solvent was removed in vacuo which afforded the desired product N-(5-bromo-4-methyl-thiazol-2-yl)-3-[[7-(5-methyl-1,2,4-oxadiazol-3-yl)-1-isoquinolyl]amino]-cyclobutanecarboxamide (227.2 mg, 55%, 0.4486 mmol) as a pale-brown solid. HPLC/MS m/z 531.1241 [M+H]⁺, Rt (U): 2.37 min. ¹H NMR (600 MHz, DMSO-d₆) δ 12.25 (s, 1H), 8.92-9.11 (m, 1H), 8.14 (dd, J=1.6, 8.5 Hz, 1H), 8.08 (d, J=7.1 Hz, 1H), 7.95 (d, J=5.7 Hz, 1H), 7.83 (d, J=8.5 Hz, 1H), 6.92-6.99 (m, 1H), 4.70 (tq, J=7.4, 9.3 Hz, 1H), 3.07 (tt, J=7.8, 9.8 Hz, 1H), 2.54-2.62 (m, 2H), 2.71 (s, 3H), 2.41 (qd, J=2.6, 9.3 Hz, 2H), 2.23 (s, 3H).

### Example 98: (1 s,3s)-N-(5-(tert-butyl)-4-methylthiazol-2-yl)-3-((7-(5-methyl-1,2,4-oxadiazol-3-yl)isoquinolin-1-yl)amino)cyclobutane-1-carboxamide

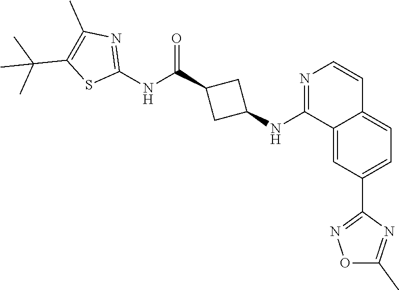 [SMILES: Cc1nc(-c2ccc3ccnc(N[C@H]4C[C@@H](C(=O)Nc5nc(C)c(C(C)(C)C)s5)C4)c3c2)no1]

Example 98.1.: 5-Methyl-3-(2-oxidoisoquinolin-2-ium-7-yl)-1,2,4-oxadiazole [Example 96](900 mg, 3.96 mmol) and methyl cis-3-amino-cyclobutanecarboxylate hydrochloride (820 mg, 4.95 mmol) were mixed in anhydrous DCM (7.9 mL) at rt under an argon atmosphere. DIPEA (3.3 mL, 18.81 mmol) and PyBroP (2.40 g, 5.15 mmol) were added, and the reaction mixture was stirred at rt overnight. Volatiles were removed under reduced pressure and the crude material was purified by flash chromatography (20-60% EtOAc in cyclohexane) to yield 1.09 g (81%) of methyl (1s,3s)-3-((7-(5-methyl-1,2,4-oxadiazol-3-yl)isoquinolin-1-yl)amino)cyclobutane-1-carboxylate as an off-white solid. HPLC/MS m/z: 339.1 [M+H]⁺, Rt (T): 1.05 min.

### Example 98.2.: Methyl 3-[[7-(5-methyl-1,2,4-oxadiazol-3-yl)-1-isoquinolyl]amino]-cyclobutanecarboxylate (1.00 g, 2.96 mmol), THF (12 mL), MeOH (6 mL) and water (12 mL) were mixed at ambient temperature. Lithium hydroxide (248 mg, 5.91 mmol) was added, and the reaction mixture was stirred for 1 h. The reaction mixture was concentrated under reduced pressure and purified by reverse flash chromatography (5-30% MeOH in water [0.1% FA]) to yield 777 mg (81%) of (1 s,3s)-3-((7-(5-methyl-1,2,4-oxadiazol-3-yl)isoquinolin-1-yl)amino)cyclobutane-1-carboxylic acid as an off-white, crystalline solid. HPLC/MS m/z: 325.1 [M+H]⁺, Rt (T): 0.97 min.

Example 98.3. Preparation as described for Example 47.5 using (1 s,3s)-3-((7-(5-methyl-1,2,4-oxadiazol-3-yl)isoquinolin-1-yl)amino)cyclobutane-1-carboxylic acid (30 mg, 0.093 mmol) and 5-tert-butyl-4-methyl-thiazol-2-ylamine (32 mg, 0.185 mmol). Purification by reverse flash chromatography followed by SCX-2 filtration. Yield: 24 mg (52%) off-white solid. HPLC/MS m/z: 477.2068 [M+H]⁺, Rt (U): 2.84 min. ¹H NMR (600 MHz, DMSO-d₆) δ 11.76 (s, 1H), 9.01 (s, 1H), 8.14 (dd, J=8.5, 1.5 Hz, 1H), 8.08 (d, J=6.9 Hz, 1H), 7.95 (d, J=5.7 Hz, 1H), 7.83 (d, J=8.5 Hz, 1H), 6.96 (d, J=5.7 Hz, 1H), 4.69-4.60 (m, 1H), 3.06-2.98 (m, 1H), 2.71 (s, 3H), 2.59-2.53 (m, 2H), 2.42-2.34 (m, 2H), 2.31 (s, 3H), 1.36 (s, 9H).

The following examples were synthesized in analogously:

### Example 99: Propyl4-methyl-2-((1s,3s)-3-((7-(5-methyl-1,2,4-oxadiazol-3-yl)isoquinolin-1-yl)amino)cyclobutane-1-carboxamido)thiazole-5-carboxylate

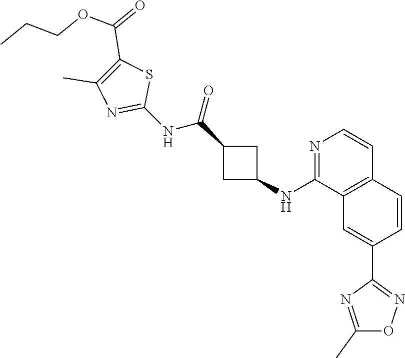 [SMILES: CCCOC(=O)c1sc(NC(=O)[C@H]2C[C@@H](Nc3nccc4ccc(-c5noc(C)n5)cc34)C2)nc1C]

Preparation as described for Example 47.5 to afford propyl 4-methyl-2-((1s,3s)-3-((7-(5-methyl-1,2,4-oxadiazol-3-yl)isoquinolin-1-yl)amino)cyclobutane-1-carboxamido)thiazole-5-carboxylate (17 mg, 45%, 0.0336 mmol). HPLC/MS m/z: 507.18, [M+H]⁺, Rt (U): 2.84 min. ¹H NMR (600 MHz, DMSO-d₆) δ 12.48 (s, 1H), 9.01 (s, 1H), 8.15 (dd, J=8.4, 1.5 Hz, 1H), 8.12-8.08 (m, 1H), 7.95 (d, J=5.7 Hz, 1H), 7.84 (d, J=8.5 Hz, 1H), 6.97 (d, J=5.7 Hz, 1H), 4.69 (ddt, J=16.8, 9.2, 7.3 Hz, 1H), 4.16 (t, J=6.5 Hz, 2H), 3.11 (ddd, J=17.5, 9.8, 7.8 Hz, 1H), 2.71 (s, 3H), 2.64-2.57 (m, 2H), 2.55 (s, 3H), 2.42 (qd, J=9.4, 2.5 Hz, 2H), 1.68 (sext, J=7.1 Hz, 2H), 0.95 (t, J=7.4 Hz, 3H).

### Example 100: (1 s,3s)-N-(5-(tert-butyl)isoxazol-3-yl)-3-((7-(5-methyl-1,2,4-oxadiazol-3-yl)isoquinolin-1-yl)amino)cyclobutane-1-carboxamide

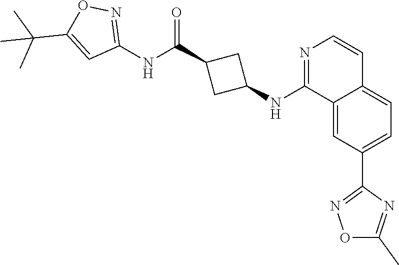 [SMILES: Cc1nc(-c2ccc3ccnc(N[C@H]4C[C@@H](C(=O)Nc5cc(C(C)(C)C)on5)C4)c3c2)no1]

Preparation as described for Example 48 using 3-[[7-(5-methyl-1,2,4-oxadiazol-3-yl)-1-isoquinolyl]amino]cyclobutanecarboxylic acid [Example 96](30 mg, 0.093 mmol) and 3-amino-5-tert-butylisoxazole (26 mg, 0.185 mmol). Purification by reverse flash chromatography followed by SCX-2 filtration. Yield: 22 mg (53%) colourless, amorphous solid. HPLC/MS m/z: 447.2133 [M+H]⁺, Rt (U): 2.62 min. ¹H NMR (600 MHz, DMSO-d₆) δ 10.88 (s, 1H), 9.02 (d, J=1.6 Hz, 1H), 8.14 (dd, J=8.5, 1.5 Hz, 1H), 8.08 (d, J=6.9 Hz, 1H), 7.95 (d, J=5.7 Hz, 1H), 7.83 (d, J=8.5 Hz, 1H), 6.96 (d, J=5.7 Hz, 1H), 6.63 (s, 1H), 4.68-4.58 (m, 1H), 3.04-2.96 (m, 1H), 2.71 (s, 3H), 2.58-2.52 (m, 2H), 2.40-2.34 (m, 2H), 1.29 (s, 9H).

### Example 101: (1s,3s)-3-((7-(5-methyl-1,2,4-oxadiazol-3-yl)isoquinolin-1-yl)amino)-N-(1-methyl-5-pentyl-1 H-pyrazol-3-yl)cyclobutane-1-carboxamide

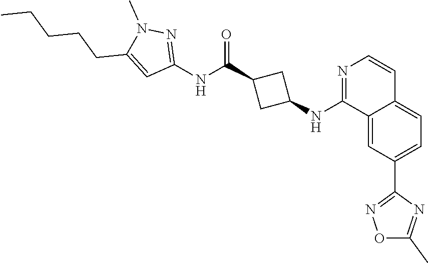 [SMILES: CCCCCc1cc(NC(=O)[C@H]2C[C@@H](Nc3nccc4ccc(-c5noc(C)n5)cc34)C2)nn1C]

Preparation as described for Example 48 using 3-[[7-(5-methyl-1,2,4-oxadiazol-3-yl)-1-isoquinolyl]amino]cyclobutanecarboxylic acid [Example 96](30 mg, 0.093 mmol) and 1-methyl-5-pentyl-pyrazol-3-amine [Example 57](23 mg, 0.139 mmol). Purification by reverse flash chromatography followed by SCX-2 filtration. Yield: 23 mg (51%) off-white, amorphous solid. HPLC/MS m/z: 474.2615 [M+H]⁺, Rt (Q): 2.72 min. ¹H NMR (600 MHz, DMSO-d₆) δ 10.18 (s, 1H), 9.03 (s, 1H), 8.14 (dd, J=8.5, 1.5 Hz, 1H), 8.08 (d, J=6.9 Hz, 1H), 7.95 (d, J=5.7 Hz, 1H), 7.83 (d, J=8.5 Hz, 1H), 6.95 (d, J=5.7 Hz, 1H), 6.32 (s, 1H), 4.66-4.56 (m, 1H), 3.61 (s, 3H), 2.96-2.89 (m, 1H), 2.71 (s, 3H), 2.55 (t, J=7.7 Hz, 2H), 2.52-2.47 (m, 2H), 2.39-2.32 (m, 2H), 1.59-1.51 (m, 2H), 1.36-1.27 (m, 4H), 0.90-0.85 (m, 3H).

### Example 102: (1s,3s)-N-(5-isobutyl-4-methylthiophen-2-yl)-3-((7-(5-methyl-1,2,4-oxadiazol-3-yl)isoquinolin-1-yl)amino)cyclobutane-1-carboxamide formate

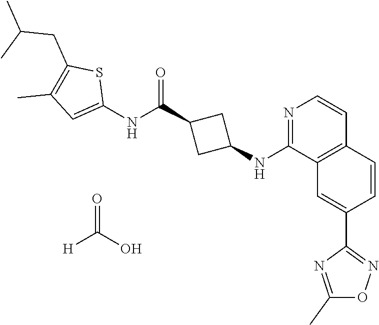 [SMILES: Cc1nc(-c2ccc3ccnc(N[C@H]4C[C@@H](C(=O)Nc5cc(C)c(CC(C)C)s5)C4)c3c2)no1 ; O=CO]

Preparation as described for 0 using 3-[[7-(5-methyl-1,2,4-oxadiazol-3-yl)-1-isoquinolyl]amino]cyclobutanecarboxylic acid [Example 96] and 5-isobutyl-4-methyl-thiophen-2-amine. Purification by reverse flash chromatography. Yield: 41 mg (35%). HPLC/MS m/z: 476.21, [M+H]⁺, Rt (U): 2.99 min. ¹H NMR (500 MHz, DMSO-d₆) δ 10.82 (s, 1H), 9.03 (d, J=1.5 Hz, 1H), 8.16 (s, 1H), 8.14 (dd, J=8.5, 1.6 Hz, 1H), 8.10 (d, J=7.1 Hz, 1H), 7.95 (d, J=5.7 Hz, 1H), 7.83 (d, J=8.5 Hz, 1H), 6.96 (dd, J=5.9, 0.9 Hz, 1H), 6.36 (s, 1H), 4.68 (sext, J=8.2 Hz, 1H), 2.95-2.83 (m, 1H), 2.71 (s, 3H), 2.58-2.50 (m, 2H), 2.46 (d, J=7.1 Hz, 2H), 2.43-2.32 (m, 2H), 2.01 (s, 3H), 1.74 (hept, J=6.7 Hz, 1H), 0.89 (d, J=6.6 Hz, 6H).

### Example 103: (1s,3s)-3-((7-(5-methyl-1,2,4-oxadiazol-3-yl)isoquinolin-1-yl)amino)-N-(4-methyl-5-(trifluoromethyl)thiazol-2-yl)cyclobutane-1-carboxamide

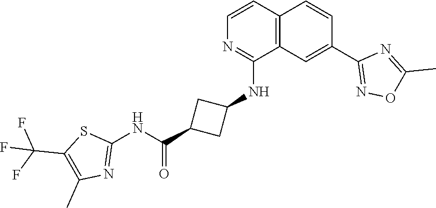 [SMILES: Cc1nc(-c2ccc3ccnc(N[C@H]4C[C@@H](C(=O)Nc5nc(C)c(C(F)(F)F)s5)C4)c3c2)no1]

Preparation as described for example Example 48 using 3-[[7-(5-methyl-1,2,4-oxadiazol-3-yl)-1-isoquinolyl]amino]cyclobutanecarboxylic acid [Example 96] and 4-methyl-5-(trifluoromethyl)thiazol-2-amine. Purification by reverse flash chromatography. Yield: 7 mg (12%). HPLC/MS m/z: 489.13, [M+H]⁺, Rt (U): 2.90 min. ¹H NMR (600 MHz, DMSO-d₆) δ 12.61 (s, 1H), 9.02-8.99 (m, 1H), 8.14 (dd, J=8.5, 1.6 Hz, 1H), 8.09 (d, J=7.1 Hz, 1H), 7.95 (d, J=5.7 Hz, 1H), 7.84 (d, J=8.5 Hz, 1H), 6.97 (dd, J=5.9, 0.8 Hz, 1H), 4.71 (ddt, J=16.7, 9.2, 7.3 Hz, 1H), 3.12 (tt, J=9.8, 7.8 Hz, 1H), 2.71 (s, 3H), 2.64-2.58 (m, 2H), 2.46-2.41 (m, 2H), 2.40 (q, J=1.8 Hz, 3H).

### Example 104: tert-Butyl 4-methyl-2-((1s,3s)-3-(3-(5-methyl-1,2,4-oxadiazol-3-yl)benzamido)cyclobutane-1-carboxamido)thiazole-5-carboxylate

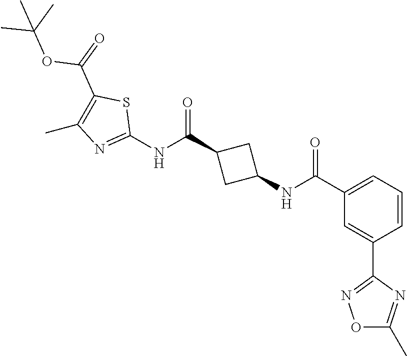 [SMILES: Cc1nc(-c2cccc(C(=O)N[C@H]3C[C@@H](C(=O)Nc4nc(C)c(C(=O)OC(C)(C)C)s4)C3)c2)no1]

Example 104.1: To 3-(tert-butoxycarbonylamino)cyclobutanecarboxylic acid (200.00 mg, 0.9292 mmol), 3-(ethyliminomethyleneamino)-N,N-dimethyl-propan-1-amine hydrochloride (356.24 mg, 1.8583 mmol), HOBt (251.09 mg, 1.8583 mmol) and tert-butyl 2-amino-4-methyl-thiazole-5-carboxylate [Example 110.1](258.83 mg, 1.2079 mmol) were dissolved in dry DMF (4.65 mL). The reaction mixture was stirred at 70° C. overnight. Purification by silica gel column chromatography (eluent: 10-70% EtOAc in cyclohexane). The material was dissolved in EtOAc and washed with HCl (1 M). Concentration in vacuo afforded tert-butyl 2-[[3-(tert-butoxycarbonylamino)-cyclobutanecarbonyl]amino]-4-methyl-thiazole-5-carboxylate (152 mg, 37%, 0.340 mmol). HPLC/MS m/z: 412.19, [M+H]⁺, Rt (P): 1.64 min.

Example 104.2.: tert-Butyl 2-[[3-(tert-butoxycarbonylamino)cyclo-butanecarbonyl]amino]-4-methyl-thiazole-5-carboxylate (152.00 mg, 0.3694 mmol) was dissolved in tert-butanol (1.34 mL). HCl in dioxane (2.77 mL, 11.081 mmol) was added and the reaction mixture was stirred at rt for 1 h. The volatiles were removed under vacuum. Purification by ion exchange SCX-II column chromatography, washing with MeOH before eluting with 2 M NH₃ in MeOH afforded tert-butyl 2-[(3-aminocyclobutanecarbonyl)amino]-4-methyl-thiazole-5-carboxylate (106 mg, 92%, 0.3404 mmol). HPLC/MS m/z: 312.15, [M+H]⁺, Rt (P): 1.20 min.

Example 104.3: 3-(5-Methyl-1,2,4-oxadiazol-3-yl)benzoic acid (15.00 mg, 0.0735 mmol) and BTFFH (30.20 mg, 0.0955 mmol) were mixed in DCM (0.15 mL) at rt under argon. DIPEA (0.06 mL, 0.3306 mmol) was added, and the reaction mixture was stirred for 30 min. tert-Butyl 2-[(3-aminocyclobutanecarbonyl)amino]-4-methyl-thiazole-5-carboxylate (22.88 mg, 0.0735 mmol) was added. The vial was capped, and the reaction was heated to 80° C. for 45 min. Purified by reverse phase column chromatography (eluent: 20-80% MeOH in water) to afford tert-butyl 4-methyl-2-((1s,3s)-3-(3-(5-methyl-1,2,4-oxadiazol-3-yl)benzamido)cyclobutane-1-carboxamido)thiazole-5-carboxylate (22 mg, 60%, 0.0442 mmol) as a colorless, amorphous solid. HPLC/MS m/z: 498.18, [M+H]⁺, Rt (U): 1.57 min. ¹H NMR (600 MHz, DMSO-d₆) δ 12.36 (br s, 1H), 9.00 (d, J=7.5 Hz, 1H), 8.52-8.49 (m, 1H), 8.16-8.12 (m, 1H), 8.09-8.06 (m, 1H), 7.66 (t, J=7.8 Hz, 1H), 4.50-4.41 (m, 1H), 3.12-3.04 (m, 1H), 2.70 (s, 3H), 2.56-2.51 (m, 5H), 2.40-2.33 (m, 2H), 1.52 (s, 9H).

The following examples were prepared analogously:

### Example 105: tert-Butyl 2-((1s,3s)-3-(3-(1,2,4-oxadiazol-3-yl)benzamido)cyclobutane-1-carboxamido)-4-methylthiazole-5-carboxylate

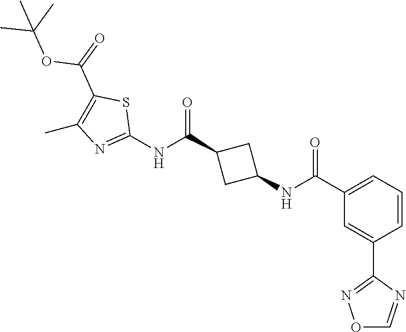 [SMILES: Cc1nc(NC(=O)[C@H]2C[C@@H](NC(=O)c3cccc(-c4ncon4)c3)C2)sc1C(=O)OC(C)(C)C]

14 mg (39%). HPLC/MS m/z: 484.16, [M+H]⁺, Rt (U): 3.22 min. ¹H NMR (600 MHz, DMSO-d₆) δ 12.40 (s, 1H), 9.78 (d, J=2.3 Hz, 1H), 9.02 (dd, J=7.4, 2.2 Hz, 1H), 8.55 (d, J=2.1 Hz, 1H), 8.24-8.16 (m, 1H), 8.12-8.06 (m, 1H), 7.69 (td, J=7.8, 2.2 Hz, 1H), 4.46 (dt, J=9.4, 7.4 Hz, 1H), 3.13-3.04 (m, 1H), 2.57-2.49 (m, 5H), 2.40-2.28 (m, 2H), 1.52 (d, J=2.4 Hz, 9H).

### Example 106: tert-Butyl 4-methyl-2-((1s,3s)-3-(3-(5-methyl-1,3,4-oxadiazol-2-yl)benzamido)cyclobutane-1-carboxamido)thiazole-5-carboxylate

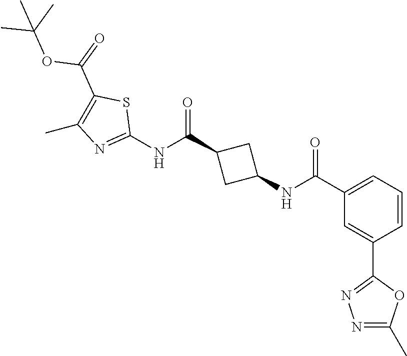 [SMILES: Cc1nnc(-c2cccc(C(=O)N[C@H]3C[C@@H](C(=O)Nc4nc(C)c(C(=O)OC(C)(C)C)s4)C3)c2)o1]

20 mg (55%) colorless, amorphous solid. HPLC/MS m/z: 498.18, [M+H]⁺, Rt (U): 3.13 min. ¹H NMR (600 MHz, DMSO-d₆) δ 12.41 (s, 1H), 9.02 (d, J=7.5 Hz, 1H), 8.48 (t, J=1.8 Hz, 1H), 8.11 (ddt, J=12.0, 7.8, 1.4 Hz, 2H), 7.70 (t, J=7.8 Hz, 1H), 4.46 (ddt, J=17.0, 9.3, 7.6 Hz, 1H), 3.08 (tt, J=9.8, 7.8 Hz, 1H), 2.62 (s, 3H), 2.56-2.47 (m, 5H), 2.37 (qd, J=9.4, 2.6 Hz, 2H), 1.52 (s, 9H).

### Example 107: tert-Butyl-4-methyl-2-((1s,3s)-3-(3-(2-methyl-2H-tetrazol-5-yl)benzamido)cyclobutane-1-carboxamido)thiazole-5-carboxylate

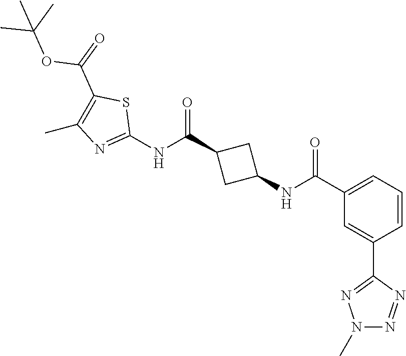 [SMILES: Cc1nc(NC(=O)[C@H]2C[C@@H](NC(=O)c3cccc(-c4nnn(C)n4)c3)C2)sc1C(=O)OC(C)(C)C]

20 mg (50%). HPLC/MS m/z: 498.19, [M+H]⁺, Rt (U): 3.20 min. ¹H NMR (600 MHz, DMSO-d₆) δ 12.39 (s, 1H), 9.00 (d, J=7.5 Hz, 1H), 8.58 (t, J=1.8 Hz, 1H), 8.20 (dt, J=7.7, 1.4 Hz, 1H), 8.04 (dt, J=7.9, 1.5 Hz, 1H), 7.66 (t, J=7.8 Hz, 1H), 4.46 (s, 4H), 3.08 (tt, J=9.8, 7.8 Hz, 1H), 2.57-2.45 (m, 5H), 2.37 (qd, J=9.4, 2.6 Hz, 2H), 1.52 (s, 9H).

### Example 108: tert-Butyl 2-((1s,3s)-3-((7-chloroisoquinolin-1-yl)amino)cyclobutane-1-carboxamido)-4-methylthiazole-5-carboxylate

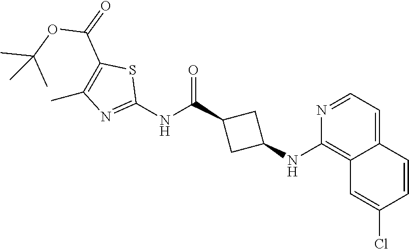 [SMILES: Cc1nc(NC(=O)[C@H]2C[C@@H](Nc3nccc4ccc(Cl)cc34)C2)sc1C(=O)OC(C)(C)C]

Example 108.1: 7-Chloroisoquinoline (0.15 g, 0.9169 mmol) was dissolved in CHCl₃ (2.86 mL) and cooled in an ice bath. mCPBA (0.24 g, 1.0885 mmol) was added. The reaction was stirred in icebath for 2 h. Warmed to rt and further CHCl₃ (1 mL) was added. K₂CO₃ (0.51 g, 3.6675 mmol) was added, and the mixture stirred for 3 h at rt before filtering through a pad of anhydrous MgSO₄. The filtrate was concentrated in vacuo to yield 7-chloro-2-oxido-isoquinolin-2-ium (0.15 g, 91%, 0.8352 mmol) as an off-white powder. HPLC/MS m/z: 180.04, [M+H]⁺, Rt (P): 0.93 min.

Example 108.2.: 7-Chloro-2-oxido-isoquinolin-2-ium (30 mg, 0.1670 mmol) was dissolved/suspended in DCM (0.70 mL) and tert-butyl 2-[(3-aminocyclobutane-carbonyl)amino]-4-methyl-thiazole-5-carboxylate [Example 104](57.22 mg, 0.1837 mmol) was added, quickly followed by DIPEA (0.09 mL, 0.5011 mmol). To this solution was added PyBroP (101.23 mg, 0.2171 mmol) and the mixture was stirred overnight at rt. Purification by silica gel column chromatography (eluent: 20-70% EtOAc in cyclohexane) followed by ion exchange SCX-II column chromatography, washing with MeOH before eluting with 2 M NH₃ in MeOH to afford tert-butyl 2-((1s,3s)-3-((7-chloroisoquinolin-1-yl)amino)cyclobutane-1-carboxamido)-4-methylthiazole-5-carboxylate (36 mg, 46%, 0.0761 mmol) as a yellowish powder. HPLC/MS m/z: 473.14, [M+H]⁺, Rt (U): 3.03 min. ¹H NMR (600 MHz, Chloroform-d) δ 10.74 (s, 1H), 8.35 (d, J=5.9 Hz, 1H), 7.76 (d, J=2.0 Hz, 1H), 7.64 (d, J=8.7 Hz, 1H), 7.55 (dd, J=8.7, 1.9 Hz, 1H), 6.99 (d, J=5.9 Hz, 1H), 5.64 (d, J=7.5 Hz, 1H), 4.64 (q, J=8.2 Hz, 1H), 3.05 (t, J=8.6 Hz, 1H), 2.82 (ddt, J=12.1, 6.8, 3.1 Hz, 4H), 2.64 (s, 3H), 1.57 (s, 9H).

The following example was prepared analogously:

### Example 109: tert-Butyl 4-methyl-2-((1s,3s)-3-((7-(trifluoromethyl)isoquinolin-1-yl)amino)cyclobutane-1-carboxamido)thiazole-5-carboxylate

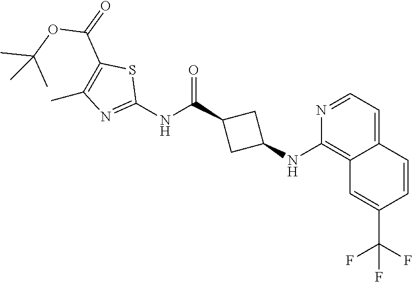 [SMILES: Cc1nc(NC(=O)[C@H]2C[C@@H](Nc3nccc4ccc(C(F)(F)F)cc34)C2)sc1C(=O)OC(C)(C)C]

16 mg (22%) yellow powder. HPLC/MS m/z: 507.17, [M+H]⁺, Rt (U): 3.17 min. ¹H NMR (600 MHz, Chloroform-d) δ 10.57 (s, 1H), 8.46 (d, J=5.9 Hz, 1H), 8.07-8.01 (m, 1H), 7.83-7.71 (m, 2H), 7.05 (dd, J=5.9, 0.9 Hz, 1H), 5.87 (d, J=7.4 Hz, 1H), 4.74-4.59 (m, 1H), 3.07 (quint, J=8.6 Hz, 1H), 2.85 (ddd, J=12.0, 6.4, 3.1 Hz, 4H), 2.64 (s, 3H), 1.57 (s, 9H).

### Example 110: tert-Butyl 4-methyl-2-[[3-[[7-(5-methyl-1,2,4-oxadiazol-3-yl)-1-isoquinolyl]amino]cyclobutanecarbonyl]amino]thiazole-5-carboxylate

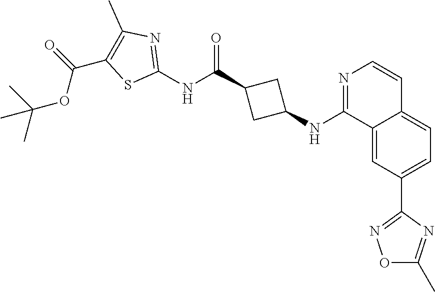 [SMILES: Cc1nc(-c2ccc3ccnc(N[C@H]4C[C@@H](C(=O)Nc5nc(C)c(C(=O)OC(C)(C)C)s5)C4)c3c2)no1]

Example 110.1: 2-Amino-4-methyl-thiazole-5-carboxylic acid (3.00 g, 18.97 mmol), DMAP (232 mg, 1.90 mmol) and 1-(3-dimethylaminopropyl)-3-ethylcarbodiimide hydrochloride (4.36 g, 22.76 mmol) were dissolved in anhydrous DMF (38 mL) under an argon atmosphere. Anhydrous tert-butanol (36 mL, 379.31 mmol) was added, and the reaction mixture was stirred at 80° C. for 2 h. The reaction mixture was concentrated under reduced pressure, mixed with water (100 mL) and extracted with EtOAc (3×75 mL). The combined organic layer was washed with saturated NaCl (3×50 mL), dried over MgSO₄ and concentrated under reduced pressure. The crude material was purified by flash chromatography (20-60% EtOAc in cyclohexane) to yield 2.10 g (52%) of tert-butyl 2-amino-4-methyl-thiazole-5-carboxylate as an off-white, crystalline solid. HPLC/MS m/z: 215.0866 [M+H]⁺, Rt (X):2.21 min.

Example 110.2.: Preparation as described for 0 using 3-[[7-(5-methyl-1,2,4-oxadiazol-3-yl)-1-isoquinolyl]amino]cyclobutanecarboxylic acid [0](30 mg, 0.093 mmol) and tert-butyl 2-amino-4-methyl-thiazole-5-carboxylate (50 mg, 0.234 mmol). Purification by reverse flash chromatography followed by SCX-2 filtration. Yield: 33 mg (53%) colourless, amorphous solid. HPLC/MS m/z: 521.1965 [M+H]⁺, Rt (U): 2.92 min. ¹H NMR (600 MHz, DMSO-d₆) δ 12.40 (s, 1H), 9.01 (s, 1H), 8.14 (dd, J=8.5, 1.6 Hz, 1H), 8.09 (d, J=7.0 Hz, 1H), 7.95 (d, J=5.7 Hz, 1H), 7.84 (d, J=8.5 Hz, 1H), 6.97 (d, J=5.7 Hz, 1H), 4.73-4.64 (m, 1H), 3.13-3.06 (m, 1H), 2.71 (s, 3H), 2.63-2.56 (m, 2H), 2.51 (s, 3H), 2.45-2.38 (m, 2H), 1.51 (s, 9H).

### Example 111: tert-Butyl 4-methyl-2-[methyl-[3-[[7-(5-methyl-1,2,4-oxadiazol-3-yl)-1-isoquinolyl]amino]cyclobutanecarbonyl]amino]thiazole-5-carboxylate

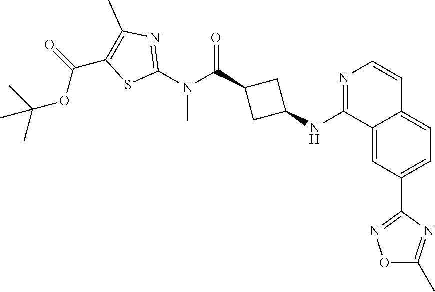 [SMILES: Cc1nc(-c2ccc3ccnc(N[C@H]4C[C@@H](C(=O)N(C)c5nc(C)c(C(=O)OC(C)(C)C)s5)C4)c3c2)no1]

Example 111.1: tert-Butyl acetoacetate (0.50 mL, 3.02 mmol) and pyridine (0.24 mL, 3.02 mmol) were dissolved in anhydrous EtOH (6.0 mL) and heated at 70° C. for 15 min. The mixture was then cooled to rt followed by addition of iodine (765 mg, 3.015 mmol) and N-methylthiourea (544 mg, 6.03 mmol). The reaction mixture was stirred at rt for 1 h. Then, the reaction mixture was heated at 50° C. overnight. The reaction mixture was cooled to rt and diluted with water (20 mL). Solid Na₂03S₂ was added and stirred for 5 min. The mixture was mixed with EtOAc (50 mL). The aqueous layer was separated, and the organic layer was washed with saturated NaCl (2×20 mL), dried over anhydrous MgSO₄ and concentrated under reduced pressure. The crude reaction mixture was purified by flash chromatography (10-80% EtOAc in cyclohexane) to yield 145 mg (17%) of a 4:1 mixture of tert-butyl 4-methyl-2-(methylamino)thiazole-5-carboxylate and tert-butyl 2-[2-(methylamino)thiazol-4-yl]acetate as an off-white solid, which was used in the next reaction without further purification. tert-Butyl 4-methyl-2-(methylamino)thiazole-5-carboxylate: HPLC/MS m/z: 173.0 [M-C₄H₈]⁺, Rt (T): 1.24 min. ¹H NMR (600 MHz, DMSO-d₆) δ 8.14 (q, J=4.9 Hz, 1H), 2.80 (d, J=4.8 Hz, 3H), 2.37 (s, 3H), 1.46 (s, 9H). tert-butyl 2-[2-(methylamino)thiazol-4-yl]acetate: 229.1 [M+H]⁺, Rt (T): 1.08 min. ¹H NMR (600 MHz, DMSO-d₆) δ 7.40 (q, J=4.8 Hz, 1H), 6.32 (s, 1H), 3.38 (d, J=0.9 Hz, 2H), 2.76 (d, J=4.8 Hz, 3H), 1.40 (s, 9H).

Example 111.2: Preparation as described for 0 using 3-[[7-(5-methyl-1,2,4-oxadiazol-3-yl)-1-isoquinolyl]amino]cyclobutanecarboxylic acid [0](34 mg, 0.104 mmol) and tert-butyl 4-methyl-2-(methylamino)thiazole-5-carboxylate (30 mg, 0.104 mmol). Purification by reverse flash chromatography. Yield: 7.3 mg (13%) colorless solid. HPLC/MS m/z: 535.2119 [M+H]⁺, Rt (U): 3.01 min. ¹H NMR (600 MHz, DMSO-d₆) δ 9.03 (s, 1H), 8.22-8.14 (m, 1H), 8.08 (br s, 1H), 7.94 (d, J=5.8 Hz, 1H), 7.87 (d, J=8.4 Hz, 1H), 7.01 (s, 1H), 4.80-4.67 (m, 1H), 3.61 (s, 3H), 3.56-3.48 (m, 1H), 2.77-2.70 (m, 2H), 2.71 (s, 3H), 2.54 (s, 3H), 2.51-2.44 (m, 2H), 1.51 (s, 9H).

### Example 112: tert-Butyl 4-chloro-2-[[3-[[7-(5-methyl-1,2,4-oxadiazol-3-yl)-1-isoquinolyl]amino]cyclobutanecarbonyl]amino]thiazole-5-carboxylate

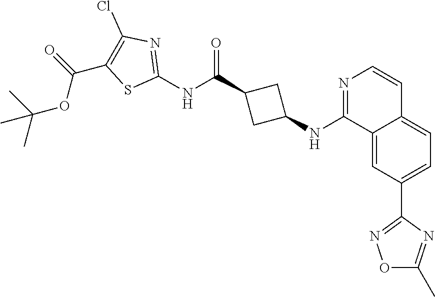 [SMILES: Cc1nc(-c2ccc3ccnc(N[C@H]4C[C@@H](C(=O)Nc5nc(Cl)c(C(=O)OC(C)(C)C)s5)C4)c3c2)no1]

Example 112.1: tert-Butyl (5-carboxy-4-chloro-1,3-thiazol-2-yl)carbamate (500 mg, 1.79 mmol) and DMAP (22 mg, 0.179 mmol) were mixed in anhydrous DMF (3.6 mL) at rt under an argon atmosphere. 1-(3-dimethylaminopropyl)-3-ethyl-carbodiimide hydrochloride (413 mg, 2.15 mmol) and anhydrous 2-methyl-2-propanol (3.4 mL, 35.9 mmol) were added successively. The reaction mixture was heated at 60 0° C. The reaction was stopped after 3 h due to the formation of two by-products. The reaction mixture was cooled to rt, mixed with 0.5 M HCl (50 mL) and extracted with EtOAc (3×30 mL). The combined organic layer was washed with saturated NaCl (3×20 mL), dried over anhydrous MgSO₄, filtered and concentrated under reduced pressure. The crude material was purified by flash chromatography (0-30% EtOAc in cyclohexane) to yield 100 mg (17%) of tert-butyl 2-(tert-butoxycarbonylamino)-4-chloro-thiazole-5-carboxylate as a colorless powder. HPLC/MS m/z: 357.0648 [M+Na]⁺, Rt (U): 3.47 min.

Example 112.2.: tert-butyl 2-(tert-butoxycarbonylamino)-4-chloro-thiazole-5-carboxylate (100 mg, 0.299 mmol) and anhydrous 2-methyl-2-propanol (1.1 mL, 11.95 mmol) were mixed at rt under an argon atmosphere. 4 M HCl in dioxane (2.2 mL, 8.96 mmol) was added and the reaction mixture was quickly cooled down in an ice bath and was then allowed to slowly warm to rt. More 4 M HCl in dioxane (0.75 mL, 2.99 mmol) was added and the mixture was continued to stir at ambient temperature. The reaction was stopped after 2 d [Note: still some starting material present but cleavage of the ester became a problem]. The reaction mixture was concentrated under reduced pressure. The crude material was used in the next reaction without further purification. HPLC/MS m/z: 179.0 [M−C₄H₈]⁺, Rt (P): 1.37 min.

Example 112.3.: Preparation as described for 0 using 3-[[7-(5-methyl-1,2,4-oxadiazol-3-yl)-1-isoquinolyl]amino]cyclobutanecarboxylic acid [0](47 mg, 0.144 mmol) and tert-butyl 2-amino-4-chloro-thiazole-5-carboxylate hydrochloride (30 mg, 0.111 mmol). Purification by reverse flash chromatography. Yield: 2.8 mg (5%) off-white solid. HPLC/MS m/z: 541.1420 [M+H]⁺, Rt (U): 2.96 min. ¹H NMR (600 MHz, Methanol-d₄) δ 8.96-8.93 (m, 1H), 8.23 (dd, J=8.4, 1.6 Hz, 1H), 7.88 (d, J=6.0 Hz, 1H), 7.81 (d, J=8.5 Hz, 1H), 7.02 (dd, J=6.1, 0.9 Hz, 1H), 4.67-4.59 (m, 1H), 3.20-3.13 (m, 1H), 2.86-2.78 (m, 2H), 2.70 (s, 3H), 2.56-2.49 (m, 2H), 1.57 (s, 9H).

### Example 113: N-tert-butyl-4-chloro-2-[[3-[[7-(5-methyl-1,2,4-oxadiazol-3-yl)-1-isoquinolyl]amino]cyclobutanecarbonyl]amino]thiazole-5-carboxamide

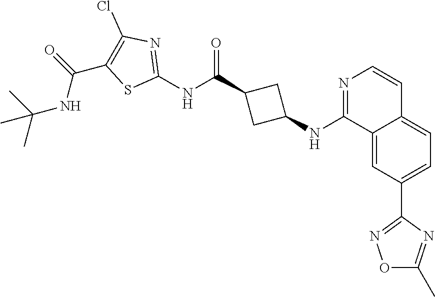 [SMILES: Cc1nc(-c2ccc3ccnc(N[C@H]4C[C@@H](C(=O)Nc5nc(Cl)c(C(=O)NC(C)(C)C)s5)C4)c3c2)no1]

Example 113.1.: tert-Butyl (5-carboxy-4-chloro-1,3-thiazol-2-yl)carbamate (480 mg, 1.72 mmol) and BTFFH (708 mg, 2.24 mmol) were mixed in anhydrous DCM (3.4 mL) in a microwave vial at rt under an argon atmosphere. DIPEA (1.36 mL, 7.75 mmol) was added, and the mixture was continued to stir at rt for 30 min. tert-Butyl amine (0.36 mL, 3.44 mmol) was added and the reaction mixture was heated at 80° C. under microwave irradiation for 1 h. The mixture was cooled to rt and volatiles were removed under reduced pressure. The crude material was purified by flash chromatography (0-40% EtOAc in cyclohexane) to yield 201 mg (35%) of tert-butyl N-[5-(tert-butylcarbamoyl)-4-chloro-thiazol-2-yl]carbamate as a colourless, amorphous solid. HPLC/MS m/z: 334.1 [M+H]⁺, Rt (U): 3.12 min.

Example 113.2.: tert-Butyl N-[5-(tert-butylcarbamoyl)-4-chloro-thiazol-2-yl]carbamate (200 mg, 0.60 mmol) and 4 M HCl in dioxane (4.5 mL, 18.0 mmol) were mixed at 0° C. under an argon atmosphere. The reaction mixture was allowed to warm to ambient temperature and was stirred for 6 h [additional 4 M HCl in dioxane (1.5 mL, 6.00 mmol) was added after 4 h]. The reaction mixture was concentrated under reduced pressure to yield 160 mg (99%) of 2-amino-N-tert-butyl-4-chloro-thiazole-5-carboxamide hydrochloride as an off-white powder. HPLC/MS m/z: 234.0648 [M+H]⁺, Rt (U): 2.36 min.

Example 113.3.: Preparation as described for 0 using 3-[[7-(5-methyl-1,2,4-oxadiazol-3-yl)-1-isoquinolyl]amino]cyclobutanecarboxylic acid [Example 96](47 mg, 0.144 mmol) and 2-amino-N-tert-butyl-4-chloro-thiazole-5-carboxamide hydrochloride (30 mg, 0.111 mmol). Purification by reverse flash chromatography followed by SCX-2 filtration. Yield: 19 mg (31%) colourless, amorphous solid. HPLC/MS m/z: 540.1576 [M+H]⁺, Rt (U): 2.77 min. ¹H NMR (600 MHz, DMSO-d₆) δ 12.59 (s, 1H), 9.02-8.99 (m, 1H), 8.14 (dd, J=8.5, 1.6 Hz, 1H), 8.09 (d, J=7.1 Hz, 1H), 7.95 (d, J=5.7 Hz, 1H), 7.84 (d, J=8.5 Hz, 1H), 7.42 (s, 1H), 6.97 (dd, J=5.9, 0.9 Hz, 1H), 4.75-4.67 (m, 1H), 3.14-3.07 (m, 1H), 2.71 (s, 3H), 2.63-2.57 (m, 2H), 2.46-2.40 (m, 2H), 1.36 (s, 9H).

### Example 114: [(1 R)-2,2,2-trifluoro-1-methyl-ethyl]4-methyl-2-[[3-[[7-(5-methyl-1,2,4-oxadiazol-3-yl)-1-isoquinolyl]amino]cyclobutanecarbonyl]amino]thiazole-5-carboxylate

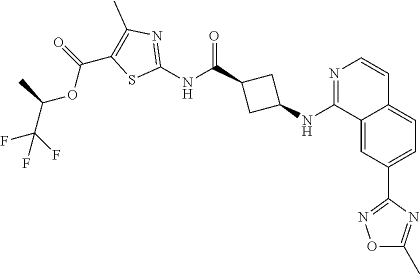 [SMILES: Cc1nc(-c2ccc3ccnc(N[C@H]4C[C@@H](C(=O)Nc5nc(C)c(C(=O)O[C@H](C)C(F)(F)F)s5)C4)c3c2)no1]

Example 114.1.: (2R)-1,1,1-Trifluoropropan-2-ol (250 mg, 2.19 mmol), DMAP (27 mg, 0.22 mmol) and 1-(3-dimethylaminopropyl)-3-ethylcarbodiimide hydrochloride (462 mg, 2.41 mmol) were mixed in anhydrous DMF (4.4 mL) at rt under an argon atmosphere. 2-Amino-4-methylthiazole-5-carboxylic acid (381 mg, 2.41 mmol) was added, and the reaction mixture was heated at 60° C. overnight. The reaction mixture was cooled to ambient temperature, mixed with water (100 mL) and extracted with EtOAc (3×50 mL). The combined organic layer was washed with saturated NaHCO₃ (50 mL), saturated NaCl (2×50 mL), dried over anhydrous MgSO₄ and concentrated under reduced pressure. The crude material was purified by reverse flash chromatography (10-70% MeOH in water [0.1% FA]) to yield 61 mg (11%) of [(1R)-2,2,2-trifluoro-1-methyl-ethyl]2-amino-4-methyl-thiazole-5-carboxylate as an off-white solid. HPLC/MS m/z: 255.0418 [M+H]⁺, Rt (U): 2.55 min.

Example 114.2.: Preparation as described for 0 using 3-[[7-(5-methyl-1,2,4-oxadiazol-3-yl)-1-isoquinolyl]amino]cyclobutanecarboxylic acid [0](30 mg, 0.093 mmol) and [(1R)-2,2,2-trifluoro-1-methyl-ethyl]2-amino-4-methyl-thiazole-5-carboxylate (26 mg, 0.102 mmol). Purification by reverse flash chromatography followed by SCX-2 filtration. Yield: 37 mg (69%) off-white solid. HPLC/MS m/z: 561.1533 [M+H]⁺, Rt (U): 2.93 min. ¹H NMR (600 MHz, DMSO-d₆) δ 12.62 (s, 1H), 9.02-8.99 (m, 1H), 8.15 (dd, J=8.5, 1.6 Hz, 1H), 8.09 (d, J=7.0 Hz, 1H), 7.96 (d, J=5.7 Hz, 1H), 7.84 (d, J=8.5 Hz, 1H), 6.99-6.95 (m, 1H), 5.63 (hept, 1H), 4.74-4.65 (m, 1H), 3.16-3.09 (m, 1H), 2.71 (s, 3H), 2.64-2.58 (m, 2H), 2.57 (s, 3H), 2.46-2.38 (m, 2H), 1.46 (d, J=6.6 Hz, 3H).

### Example 115: [(1 S)-2,2,2-trifluoro-1-methyl-ethyl]4-methyl-2-[[3-[[7-(5-methyl-1,2,4-oxadiazol-3-yl)-1-isoquinolyl]amino]cyclobutanecarbonyl]amino]thiazole-5-carboxylate

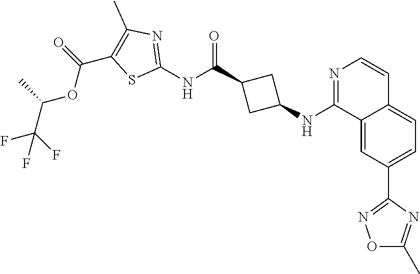 [SMILES: Cc1nc(-c2ccc3ccnc(N[C@H]4C[C@@H](C(=O)Nc5nc(C)c(C(=O)O[C@@H](C)C(F)(F)F)s5)C4)c3c2)no1]

Example 115.1.: Preparation as described in 0 using (2S)-1,1,1-trifluoropropan-2-ol (250 mg, 2.19 mmol) and 2-amino-4-methylthiazole-5-carboxylic acid (381 mg, 2.41 mmol). Purification by reverse flash chromatography. Yield: 69 mg (12%) off-white solid. HPLC/MS m/z: 255.0418 [M+H]⁺, Rt (U): 2.55 min.

Example 115.2.: Preparation as described for 0 using 3-[[7-(5-methyl-1,2,4-oxadiazol-3-yl)-1-isoquinolyl]amino]cyclobutanecarboxylic acid [0](30 mg, 0.093 mmol) and [(1S)-2,2,2-trifluoro-1-methyl-ethyl]2-amino-4-methyl-thiazole-5-carboxylate (26 mg, 0.102 mmol). Purification by reverse flash chromatography followed by SCX-2 filtration. Yield: 41 mg (75%) off-white solid. HPLC/MS m/z: 561.1528 [M+H]⁺, Rt (U): 2.92 min. ¹H NMR (600 MHz, DMSO-d₆) δ 12.62 (s, 1H), 9.02-8.99 (m, 1H), 8.14 (dd, J=8.5, 1.6 Hz, 1H), 8.09 (d, J=7.0 Hz, 1H), 7.96 (d, J=5.7 Hz, 1H), 7.84 (d, J=8.5 Hz, 1H), 6.97 (dd, J=5.9, 0.8 Hz, 1H), 5.67-5.59 (m, 1H), 4.74-4.65 (m, 1H), 3.16-3.08 (m, 1H), 2.71 (s, 3H), 2.64-2.58 (m, 2H), 2.57 (s, 3H), 2.46-2.38 (m, 2H), 1.46 (d, J=6.6 Hz, 3H).

### Example 116: Ethyl-4-(difluoromethyl)-2-[[3-[[7-(5-methyl-1,2,4-oxadiazol-3-yl)-1-isoquinolyl]amino]cyclobutanecarbonyl]amino]thiazole-5-carboxylate

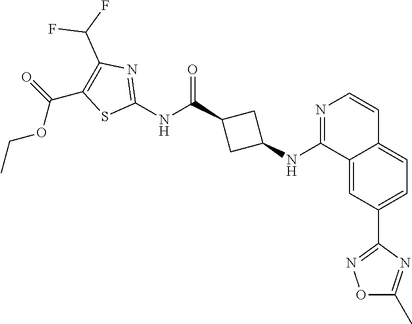 [SMILES: CCOC(=O)c1sc(NC(=O)[C@H]2C[C@@H](Nc3nccc4ccc(-c5noc(C)n5)cc34)C2)nc1C(F)F]

Example 116.1.: Ethyl 2-chloro-4,4-difluoroacetoacetate (200 mg, 1.00 mmol) and thiourea (151 mg, 2.00 mmol) were mixed in anhydrous EtOH (1.00 mL) in a microwave vial under an argon atmosphere. The reaction mixture was heated at 100° C. by microwave irradiation for 1 h. The reaction mixture was concentrated under reduced pressure and mixed with diethyl ether. The formed precipitate was filtered off, washed with diethyl ether and dried under reduced pressure to yield 111 mg (50%) of ethyl 2-amino-4-(difluoromethyl)thiazole-5-carboxylate as an off-white solid. HPLC/MS m/z: 223.0349 [M+H]⁺, Rt (X): 2.04 min.

Example 116.2.: Preparation as described for 0 using 3-[[7-(5-methyl-1,2,4-oxadiazol-3-yl)-1-isoquinolyl]amino]cyclobutanecarboxylic acid [0](30 mg, 0.093 mmol) and ethyl 2-amino-4-(difluoromethyl)thiazole-5-carboxylate (23 mg, 0.102 mmol). Purification by reverse flash chromatography. Yield: 31 mg (63%) colorless powder. HPLC/MS m/z: 529.1456 [M+H]⁺, Rt (X): 2.86 min. ¹H NMR (600 MHz, DMSO-d₆) δ 12.93 (br s, 1H), 9.02-8.99 (m, 1H), 8.15 (dd, J=8.5, 1.5 Hz, 1H), 8.09 (d, J=7.0 Hz, 1H), 7.96 (d, J=5.7 Hz, 1H), 7.84 (d, J=8.5 Hz, 1H), 7.42 (t, J=53.7 Hz, 1H), 6.97 (d, J=5.7 Hz, 1H), 4.74-4.67 (m, 1H), 4.32 (q, J=7.1 Hz, 2H), 3.16-3.09 (m, 1H), 2.71 (s, 3H), 2.65-2.59 (m, 2H), 2.47-2.41 (m, 2H), 1.31 (t, J=7.1 Hz, 3H).

### Example 117: N-(5-tert-butyl-4-methyl-thiazol-2-yl)-3-[(7-methyl-1-isoquinolyl)amino]cyclobutanecarboxamide

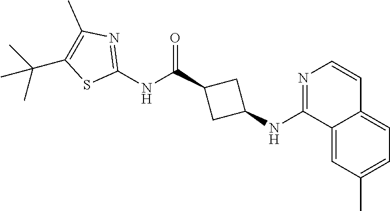 [SMILES: Cc1ccc2ccnc(N[C@H]3C[C@@H](C(=O)Nc4nc(C)c(C(C)(C)C)s4)C3)c2c1]

Example 117.1.: cis-3-Aminocyclobutanecarboxylic acid hydrochloride (463 mg, 3.05 mmol) and sodium carbonate (1.08 g, 10.2 mmol) were mixed in anhydrous NMP (4.07 mL) under an argon atmosphere and stirred at rt for 10 min. 1-Chloro-7-methylisoquinoline (362 mg, 2.04 mmol) was added and the reaction mixture was heated at 170° C. the reaction was stopped after 48 h due to the formation of by-products. The reaction mixture was purified by reverse flash chromatography (5-50% MeOH in water [0.1% FA]) to yield 98 mg (17%) of 3-[(7-methyl-1-isoquinolyl)amino]cyclobutanecarboxylic acid as an off-white solid. HPLC/MS m/z: 257.1 [M+H]⁺, Rt (U): 1.71 min.

Example 117.2.: Preparation as described for 0 using 3-[(7-methyl-1-isoquinolyl)amino]cyclobutanecarboxylic acid (30 mg, 0.117 mmol) and 5-tert-butyl-4-methyl-thiazol-2-ylamine (40 mg, 0.234 mmol). Purification by reverse flash chromatography followed by SCX-2 filtration. Yield: 17 mg (35%) colourless, amorphous solid. HPLC/MS m/z: 409.2048 [M+H]⁺, Rt (U): 2.90 min. ¹H NMR (600 MHz, DMSO-d₆) δ 11.76 (s, 1H), 8.13 (s, 1H), 7.78 (d, J=5.8 Hz, 1H), 7.59 (d, J=8.3 Hz, 1H), 7.47-7.41 (m, 2H), 6.85 (d, J=5.8 Hz, 1H), 4.65-4.56 (m, 1H), 3.06-2.97 (m, 1H), 2.59-2.51 (m, 3H), 2.47 (s, 3H), 2.35-2.26 (m, 5H), 1.36 (s, 9H).

### Example 118: 3-(5-methyl-1,2,4-oxadiazol-3-yl)-N-(3-((4-methyl-5-(3-methyl-1,2,4-oxadiazol-5-yl)thiazol-2-yl)amino)-3-oxopropyl)benzamide

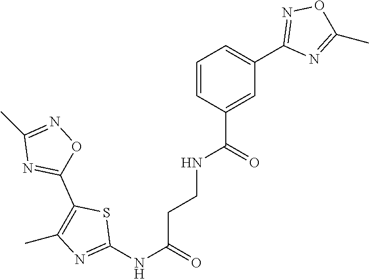

Example 118.1.: Methyl 4-methyl-2-[3-[[3-(5-methyl-1,2,4-oxadiazol-3-yl)benzoyl]amino]propanoylamino]thiazole-5-carboxylate [0](60.00 mg, 0.1397 mmol) was suspended in MeOH (1.00 mL) and water (1.00 mL) before sodium hydroxide (111.77 mg, 2.7943 mmol) was added. The reaction was heated to 55° C. but no dissolution. THF (1.00 mL) was added, and the reaction mixture became homogenous and was stirred at 55° C. for 1 hr 20 min. LCMS showed near complete conversion and some degredation product. The mixture was cooled in an icebath and acidified with 1 M HCl (˜4 mL) and concentrated in vacuo. The crude was partitioned between EtOAc (25 mL) and water (25 mL). The organic layer was washed with brine and dried over MgSO₄. Concentration in vaccuo gave a colorless powder of 4-methyl-2-[3-[[3-(5-methyl-1,2,4-oxadiazol-3-yl)benzoyl]amino]propano-ylamino]thiazole-5-carboxylic acid (14 mg, 24%, 0.0337 mmol). Purity 90% (UV). This material was used in the next step without further purification. HPLC/MS m/z: 416.1 [M+H]⁺, Rt (P): 1.81 min.

Example 118.2.: To 4-methyl-2-[3-[[3-(5-methyl-1,2,4-oxadiazol-3-yl)benzoyl]amino]propanoylamino]thiazole-5-carboxylic acid (14.00 mg, 0.0337 mmol) in anhydrous DMF (0.70 mL) was added EDC.HCl (7.43 mg, 0.0388 mmol) and HOBt (5.46 mg, 0.0404 mmol). The reaction mixture was stirred under nitrogen for 30 min at rt before the addition of acetamide oxime (3.25 mg, 0.0438 mmol). The reaction mixture was stirred ar rt for 17 h where upon LCMS showed good conversion to [(Z)-1-aminoethylideneamino]4-methyl-2-[3-[[3-(5-methyl-1,2,4-oxadiazol-3-yl)benzoyl]amino]propanoylamino]thiazole-5-carboxylate. A condenser was added, and the reaction was then heated to 100° C. behind a blast shield under nitrogen for 21 h to perform the cyclisation. The volatiles were removed in vacuo and the crude purified by reverse phase column chromatography (eluent: 20-100% MeOH in water (+0.1% formic acid modifier in both)) to afford 3-(5-methyl-1,2,4-oxadiazol-3-yl)-N-(3-((4-methyl-5-(3-methyl-1,2,4-oxadiazol-5-yl)thiazol-2-yl)amino)-3-oxopropyl)benzamide (1.2 mg, 8%, 0.0026 mmol). HPLC/MS m/z: 454.1 [M+H]⁺, Rt (R): 1.31 min. ¹H NMR (500 MHz, Acetone-d₆) δ 11.42 (broad s, 1H), 8.54 (td, J=1.8, 0.6 Hz, 1H), 8.30 (s, 1H), 8.18 (ddd, J=7.7, 1.7, 1.1 Hz, 1H), 8.09 (ddd, J=7.8, 1.9, 1.2 Hz, 1H), 7.65 (td, J=7.8, 0.6 Hz, 1H), 3.90-3.80 (m, 2H), 3.02 (t, J=6.5 Hz, 2H), 2.68 (s, 3H), 2.64 (s, 3H), 2.38 (s, 3H).

### Example 119: 3-((7-(5-methyl-1,2,4-oxadiazol-3-yl)isoquinolin-1-yl)amino)-N-(5-(5-propyl-1,2,4-oxadiazol-3-yl)thiazol-2-yl)propenamide

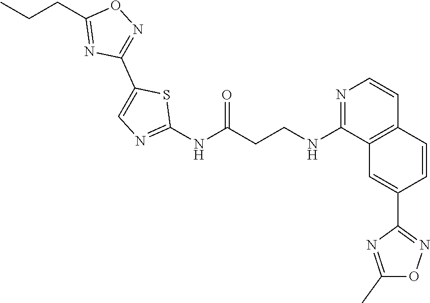 [SMILES: CCCc1nc(-c2cnc(NC(=O)CCNc3nccc4ccc(-c5noc(C)n5)cc34)s2)no1]

Example 119.1.: N-(5-cyanothiazol-2-yl)-3-[[7-(5-methyl-1,2,4-oxadiazol-3-yl)-1-isoquinolyl]amino]propanamide [0](37.40 mg, 0.0922 mmol) and TEA (0.01 mL, 0.0922 mmol) were heated at 80° C. in [bmim]OAc (0.18 mL). Hydroxylamine hydrochloride (12.82 mg, 0.1845 mmol) was then added, and the resulting solution was stirred for 1.5 h. The solution was cooled down to rt and was partitioned between water (15 mL) and EtOAc (10 mL). Aqueous phase extracted with EtOAc (2×10 mL). Organic layer was combined, dried over magnesium sulfate and concentrated under vacuum to yield N-[5-[(Z)-N′-hydroxycarbamimidoyl]thiazol-2-yl]-3-[[7-(5-methyl-1,2,4-oxadiazol-3-yl)-1-isoquinolyl]amino]propanamide (40 mg, 99%, 0.0912 mmol) as a colorless solid. HPLC/MS m/z: 439.14, [M+H]⁺, Rt (P): 1.00 min.

Example 119.2.: Butyric acid (0.01 mL, 0.0597 mmol), N-[5-[(Z)-N′-hydroxycarbamimidoyl]thiazol-2-yl]-3-[[7-(5-methyl-1,2,4-oxadiazol-3-yl)-1-isoquinolyl]amino]propanamide (21.80 mg, 0.0497 mmol), and EDC.HCl (11.44 mg, 0.0597 mmol) were suspended in a mixture of MeCN (0.17 mL) and THF (0.17 mL) under nitrogen atmosphere. The resulting solution was stirred at rt for 17 h. Volatiles were removed under reduced pressure to afford N-[(Z)-N-hydroxy-C-[2-[3-[[7-(5-methyl-1,2,4-oxadiazol-3-yl)-1-isoquinolyl]amino]propanoylamino]thiazol-5-yl]carbonimidoyl]butanamide. HPLC/MS m/z: 509.18, [M+H]⁺, Rt (P): 1.25 min.

Example 119.3.: To a solution of N-[(Z)-N-hydroxy-C-[2-[3-[[7-(5-methyl-1,2,4-oxadiazol-3-yl)-1-isoquinolyl]amino]propanoylamino]thiazol-5-yl]carbonimidoyl]butanamide (25.29 mg, 0.0497 mmol) in DMSO (0.30 mL) was added potassium hydroxide (11.16 mg, 0.1989 mmol). The resulting solution was stirred at rt for 1.5 h. Additional potassium hydroxide (4 eq.) was added, and the resulting solution was stirred at rt for 1.5 h. Purified by reverse phase column chromatography (eluent: 30-80% MeOH in water (+0.1% formic acid)) followed by purification by ion exchange SCX-II column chromatography, washing with MeOH before eluting with 2M NH₃ in MeOH to afford 3-((7-(5-methyl-1,2,4-oxadiazol-3-yl)isoquinolin-1-yl)amino)-N-(5-(5-propyl-1,2,4-oxadiazol-3-yl)thiazol-2-yl)propanamide (7 mg, 29%, 0.0143 mmol) as a colorless solid. HPLC/MS m/z: 491.16, [M+H]⁺, Rt (U): 2.62 min. ¹H NMR (600 MHz, DMSO-d₆) δ 12.65 (s, 1H), 8.91-8.87 (m, 1H), 8.16 (dd, J=8.5, 1.5 Hz, 1H), 8.11 (s, 1H), 8.06 (t, J=5.5 Hz, 1H), 7.98 (d, J=5.7 Hz, 1H), 7.86 (d, J=8.5 Hz, 1H), 7.00-6.97 (m, 1H), 3.84 (q, J=6.4 Hz, 2H), 2.95 (dt, J=10.0, 7.1 Hz, 4H), 2.70 (s, 3H), 1.80 (sext, J=7.4 Hz, 2H), 0.98 (t, J=7.4 Hz, 3H).

### Example 120: Propyl-1-methyl-3-(3-(3-(5-methyl-1,2,4-oxadiazol-3-yl)benzamido)propanamido)-1 H-pyrazole-5-carboxylate

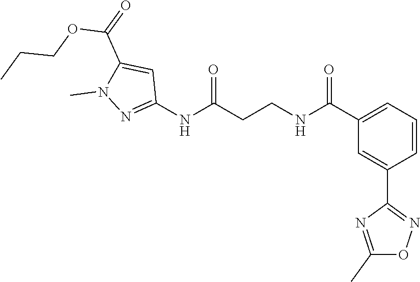 [SMILES: CCCOC(=O)c1cc(NC(=O)CCNC(=O)c2cccc(-c3noc(C)n3)c2)nn1C]

3-[[3-(5-methyl-1,2,4-oxadiazol-3-yl)benzoyl]amino]propanoic acid [0](49.58 mg, 0.1801 mmol), propyl 5-amino-2-methyl-pyrazole-3-carboxylate (30.00 mg, 0.1637 mmol) and HATU (87.17 mg, 0.2292 mmol) were dissolved in dry DMF (1.09 mL) at rt. DIPEA (0.04 mL, 0.2456 mmol) was added and the reaction mixture was stirred overnight at rt. The mixture was diluted with EtOAc and washed with a solution of NH₄Cl, water (2×), dried over MgSO₄ and concentrated in vacuo. Purified by reverse phase column chromatography (eluent: 30-100% MeOH/H₂O+0.1% formic acid) to give propyl 1-methyl-3-(3-(3-(5-methyl-1,2,4-oxadiazol-3-yl)-benzamido)propanamido)-1 H-pyrazole-5-carboxylate (54 mg, 75%, 0.1226 mmol) as a colorless powder. HPLC/MS m/z: 441.2, [M+H]⁺, Rt (R): 1.29 min. ¹H NMR (500 MHz, DMSO-d₆) δ 10.68 (s, 1H, NH), 8.81 (t, J=5.5 Hz, 1H, NH), 8.47 (t, J=1.8 Hz, 1H), 8.12 (dt, J=7.8, 1.4 Hz, 1H), 8.03 (dt, J=7.9, 1.5 Hz, 1H), 7.65 (t, J=7.8 Hz, 1H), 7.05 (s, 1H), 4.20 (t, J=6.5 Hz, 2H), 3.98 (s, 3H), 3.58-3.51 (m, 2H), 2.68 (s, 3H), 2.63 (t, J=7.0 Hz, 2H), 1.73-1.65 (m, 2H), 0.95 (t, J=7.4 Hz, 3H).

The following example was prepared analogously:

### Example 121: Propyl-4-methyl-2-(N-methyl-3-(3-(5-methyl-1,2,4-oxadiazol-3-yl)benzamido)propanamido)thiazole-5-carboxylate

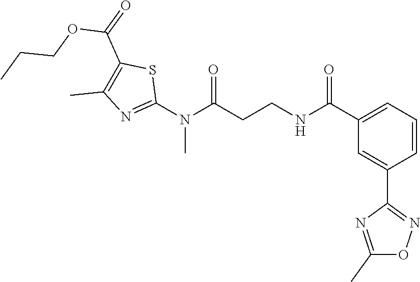 [SMILES: CCCOC(=O)c1sc(N(C)C(=O)CCNC(=O)c2cccc(-c3noc(C)n3)c2)nc1C]

15 mg (14%), colorless powder. HPLC/MS m/z: 472.2, [M+H]⁺, Rt (R): 1.42 min. ¹H NMR (500 MHz, DMSO-d₆) δ 8.85-8.79 (m, 1H), 8.50-8.45 (m, 1H), 8.13 (dt, J=7.8, 1.5 Hz, 1H), 8.04 (dt, J=7.9, 1.4 Hz, 1H), 7.66 (t, J=7.8 Hz, 1H), 4.16 (t, J=6.5 Hz, 2H), 3.66 (s, 3H), 3.64-3.59 (m, 2H), 3.07 (t, J=6.8 Hz, 2H), 2.68 (s, 3H), 2.57 (s, 3H), 1.74-1.60 (m, 2H), 0.94 (t, J=7.4 Hz, 3H).

### Example 122: Cyclopentyl-4-methyl-2-(3-(3-(5-methyl-1,2,4-oxadiazol-3-yl)benzamido)propanamido)thiazole-5-carboxylate

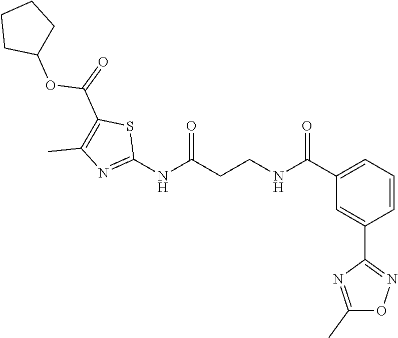 [SMILES: Cc1nc(-c2cccc(C(=O)NCCC(=O)Nc3nc(C)c(C(=O)OC4CCCC4)s3)c2)no1]

4-methyl-2-[3-[[3-(5-methyl-1,2,4-oxadiazol-3-yl)benzoyl]amino]propanoylamino]thiazole-5-carboxylic acid [0](30.00 mg, 0.0722 mmol) was dissolved in dry DCM (1.00 mL) at rt. Oxalyl dichloride (0.06 mL, 0.7222 mmol) followed by a few drops of DMF were added. The reaction mixture was stirred at rt for 2 h before the solvent was removed under reduced pressure, and cyclopentanol (1.00 mL) was added. The reaction mixture was stirred at rt for 4 h. The solvent was removed under reduced pressure and the residue was purified by reverse phase column chromatography (eluent: 30-100% MeOH/H₂O+0.1% formic acid) to afford cyclopentyl 4-methyl-2-(3-(3-(5-methyl-1,2,4-oxadiazol-3-yl)benzamido)propanamido)thiazole-5-carboxylate (8 mg, 23%, 0.0165 mmol) as an off-white powder. HPLC/MS m/z: 484.2, [M+H]⁺, Rt (R): 1.43 min. ¹H NMR (500 MHz, Acetone-d₆) δ 11.27 (s, 1H), 8.53 (t, J=1.5 Hz, 1H), 8.29-8.21 (m, 1H), 8.20-8.14 (m, 1H), 8.09-8.04 (m, 1H), 7.66-7.59 (m, 1H), 5.34-5.28 (m, 1H), 3.86-3.80 (m, 2H), 3.00-2.93 (m, 2H), 2.67 (s, 3H), 2.51 (s, 3H), 1.96-1.89 (m, 2H), 1.81-1.74 (m, 4H), 1.68-1.62 (m, 2H).

### Example 123: Hexyl-4-methyl-2-(3-(3-(5-methyl-1,2,4-oxadiazol-3-yl)benzamido)propanamido)thiazole-5-carboxylate

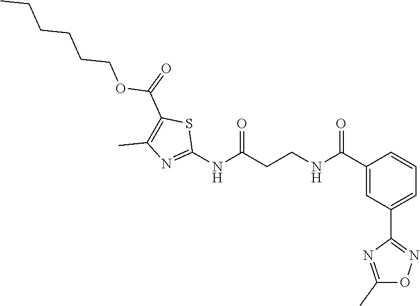 [SMILES: CCCCCCOC(=O)c1sc(NC(=O)CCNC(=O)c2cccc(-c3noc(C)n3)c2)nc1C]

Example 123.1.: 4-Methyl-2-[3-[[3-(5-methyl-1,2,4-oxadiazol-3-yl)benzoyl]amino]propanoylamino]thiazole-5-carboxylic acid [0](60.00 mg, 0.1444 mmol) was dissolved in dry DMF (1.00 mL) at rt. Pyridine (0.07 mL, 0.1733 mmol) and (2,3,4,5,6-pentafluorophenyl) 2,2,2-trifluoroacetate (0.03 mL, 0.1733 mmol) were added and the mixture was stirred for 1 h at rt and overnight at 60 0° C. The reaction mixture was diluted with EtOAc, washed with water, dried over MgSO₄ and concentrated under reduced pressure. Purified by reverse phase column chromatography (eluent: 30-100% MeOH/H₂O+0.1% formic acid) to give (2,3,4,5,6-pentafluorophenyl) 4-methyl-2-[3-[[3-(5-methyl-1,2,4-oxadiazol-3-yl)benzoyl]amino]propanoylamino]thiazole-5-carboxylate (28 mg, 33%, 0.0482 mmol) as a colorless powder. HPLC/MS m/z: 582.1, [M+H]⁺, Rt (Q): 3.29 min.

Example 123.2.: Hexan-1-ol (0.60 mL, 4.7803 mmol) was added to (2,3,4,5,6-pentafluorophenyl) 4-methyl-2-[3-[[3-(5-methyl-1,2,4-oxadiazol-3-yl)benzoyl]amino]propanoylamino]thiazole-5-carboxylate (20.00 mg, 0.0344 mmol) at rt. DMF (0.50 mL) was added. The reaction mixture was stirred at rt for 20 min, at 65° C. for 2 h and 100° C. for 3 h. DMAP (3 mg) was added, and the reaction mixture was stirred at 100° C. for 1 h. The reaction mixture was diluted with EtOAc, washed with water, dried over MgSO4 and concentrated under reduced pressure. Purified by reverse phase column chromatography (eluent: 30-100% MeOH/H₂O+0.1% formic acid) to afford hexyl 4-methyl-2-(3-(3-(5-methyl-1,2,4-oxadiazol-3-yl)benzamido)propanamido)thiazole-5-carboxylate (14 mg, 81%, 0.0280 mmol) as a colorless powder. HPLC/MS m/z: 500.2, [M+H]⁺, Rt (R): 1.54 min. ¹H NMR (500 MHz, DMSO-d₆) δ 12.54 (s, 1H), 8.86 (t, J=5.5 Hz, 1H), 8.46 (t, J=1.7 Hz, 1H), 8.13 (dt, J=7.7, 1.4 Hz, 1H), 8.02 (dt, J=8.0, 1.4 Hz, 1H), 7.65 (t, J=7.8 Hz, 1H), 4.19 (t, J=6.5 Hz, 2H), 3.65-3.56 (m, 2H), 2.78 (t, J=6.8 Hz, 2H), 2.68 (s, 3H), 2.53 (s, 3H), 1.69-1.60 (m, 2H), 1.40-1.33 (m, 2H), 1.33-1.24 (m, 4H), 0.90-0.82 (m, 3H).

The following examples were prepared analogously:

### Example 124: Cyclohexyl-4-methyl-2-(3-(3-(5-methyl-1,2,4-oxadiazol-3-yl)benzamido)propanamido)thiazole-5-carboxylate

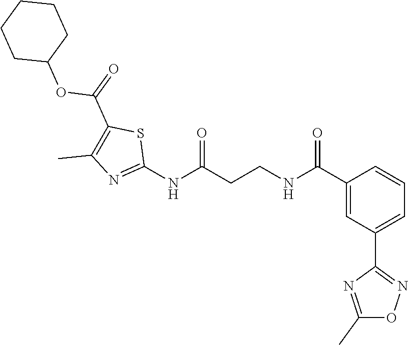 [SMILES: Cc1nc(-c2cccc(C(=O)NCCC(=O)Nc3nc(C)c(C(=O)OC4CCCCC4)s3)c2)no1]

11 mg (36%), colorless powder. HPLC/MS m/z: 498.2, [M+H]⁺, Rt (R): 1.49 min. ¹H NMR (500 MHz, DMSO-d₆) δ 12.52 (s, 1H), 8.86 (t, J=5.5 Hz, 1H), 8.46 (t, J=1.8 Hz, 1H), 8.13 (dt, J=7.7, 1.4 Hz, 1H), 8.02 (dt, J=7.9, 1.3 Hz, 1H), 7.65 (t, J=7.8 Hz, 1H), 4.92-4.84 (m, 1H), 3.64-3.56 (m, 2H), 2.78 (t, J=6.8 Hz, 2H), 2.68 (s, 3H), 2.53 (s, 3H), 1.87-1.78 (m, 2H), 1.72-1.63 (m, 2H), 1.56-1.45 (m, 3H), 1.44-1.31 (m, 3H).

### Example 125: 4-aminobutyl 4-methyl-2-(3-(3-(5-methyl-1,2,4-oxadiazol-3-yl)benzamido)propanamido)thiazole-5-carboxylate hydrochloride

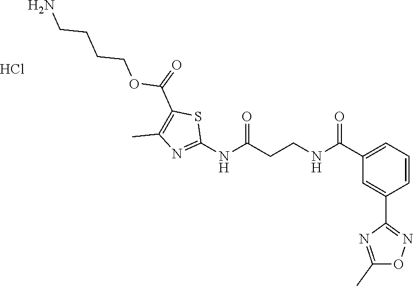 [SMILES: Cl ; Cc1nc(-c2cccc(C(=O)NCCC(=O)Nc3nc(C)c(C(=O)OCCCCN)s3)c2)no1]

tert-butyl N-(4-hydroxybutyl)carbamate was used in an analogous procedure followed by deprotection using HCl in dioxane (1.04 mL, 50 eq., 4.1762 mmol) at rt for 3 h. The solvent was removed under reduced pressure to afford 4-aminobutyl 4-methyl-2-(3-(3-(5-methyl-1,2,4-oxadiazol-3-yl)benzamido)propanamido)thiazole-5-carboxylate (43 mg, 98%, 0.0822 mmol) hydrochloride as a cream-coloured powder. HPLC/MS m/z: 487.2, [M+H]⁺, Rt (R): 0.98 min. ¹H NMR (500 MHz, Methanol-d₄) δ 8.49 (t, J=1.7 Hz, 1H), 8.19 (dt, J=7.8, 1.4 Hz, 1H), 7.99-7.94 (m, 1H), 7.61 (t, J=7.8 Hz, 1H), 4.32 (t, J=5.8 Hz, 2H), 3.77 (t, J=6.8 Hz, 2H), 3.05-2.97 (m, 2H), 2.88 (t, J=6.8 Hz, 2H), 2.66 (s, 3H), 2.59 (s, 3H), 1.88-1.77 (m, 4H).

### Example 126: 3-(4-methoxyphenyl)propyl 4-methyl-2-(3-(3-(5-methyl-1,2,4-oxadiazol-3-yl)benzamido)propanamido)thiazole-5-carboxylate

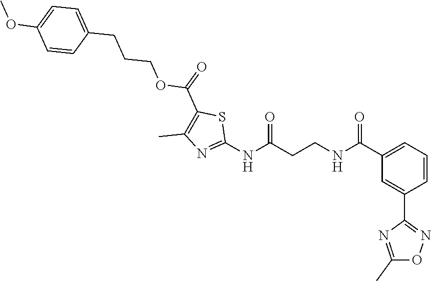 [SMILES: COc1ccc(CCCOC(=O)c2sc(NC(=O)CCNC(=O)c3cccc(-c4noc(C)n4)c3)nc2C)cc1]

20 mg (67%), colorless powder. HPLC/MS m/z: 564.2 [M+H]⁺, Rt (R): 1.52 min. ¹H NMR (500 MHz, DMSO-d₆) δ 12.55 (s, 1H), 8.87 (t, J=5.6 Hz, 1H), 8.48-8.43 (m, 1H), 8.15-8.09 (m, 1H), 8.06-7.99 (m, 1H), 7.69-7.61 (m, 1H), 7.16-7.09 (m, 2H), 6.88-6.81 (m, 2H), 4.16 (t, J=6.4 Hz, 2H), 3.71 (s, 3H), 3.60 (q, J=6.5 Hz, 2H), 2.79 (t, J=6.8 Hz, 2H), 2.68 (s, 3H), 2.66-2.59 (m, 2H), 2.54 (s, 3H), 1.97-1.89 (m, 2H).

### Example 127: 4-(2-methoxyethoxy)butyl 4-methyl-2-(3-(3-(5-methyl-1,2,4-oxadiazol-3-yl)benzamido)propanamido)thiazole-5-carboxylate

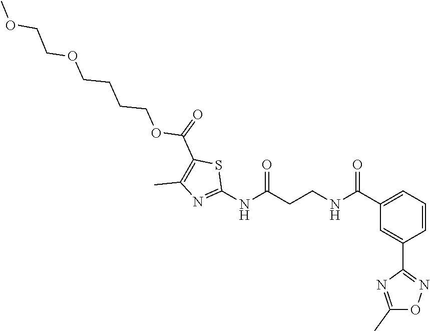 [SMILES: COCCOCCCCOC(=O)c1sc(NC(=O)CCNC(=O)c2cccc(-c3noc(C)n3)c2)nc1C]

23 mg (61%), cream coloured powder. HPLC/MS m/z: 546.2, [M+H]⁺, Rt (R): 1.35 min. ¹H NMR (500 MHz, Methanol-d₄) δ 8.49-8.47 (m, 1H), 8.19-8.15 (m, 1H), 7.96 (ddd, J=7.9, 1.9, 1.2 Hz, 1H), 7.60 (td, J=7.8, 0.6 Hz, 1H), 4.27 (t, J=6.4 Hz, 2H), 3.77 (t, J=6.7 Hz, 2H), 3.60-3.56 (m, 2H), 3.55-3.51 (m, 4H), 3.35 (s, 3H), 2.86 (t, J=6.7 Hz, 2H), 2.66 (s, 3H), 2.56 (s, 3H), 1.84-1.77 (m, 2H), 1.74-1.67 (m, 2H).

### Example 128: Methyl-2-methyl-5-[3-[[3-(5-methyl-1,2,4-oxadiazol-3-yl)benzoyl]amino]propanoylamino]pyrazole-3-carboxylate

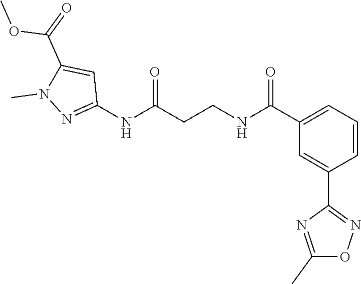 [SMILES: COC(=O)c1cc(NC(=O)CCNC(=O)c2cccc(-c3noc(C)n3)c2)nn1C]

42 mg (56%), colorless fluffy powder. HPLC/MS m/z: 413.16 [M+H]⁺, Rt (R): 1.15 min. ¹H NMR (500 MHz, DMSO-d₆) δ 10.68 (s, 1H), 8.82 (t, J=5.5 Hz, 1H), 8.52-8.43 (m, 1H), 8.16-8.10 (m, 1H), 8.03 (ddd, J=7.8, 1.8, 1.1 Hz, 1H), 7.65 (t, J=7.8 Hz, 1H), 7.05 (s, 1H), 3.99 (s, 3H), 3.83 (s, 3H), 3.55 (q, J=6.8 Hz, 2H), 2.69 (s, 3H), 2.63 (t, J=7.1 Hz, 2H).

### Example 129: 3-(5-methyl-1,2,4-oxadiazol-3-yl)-N-(3-((4-methyl-5-(pentan-2-yl)thiazol-2-yl)amino)-3-oxopropyl)benzamide

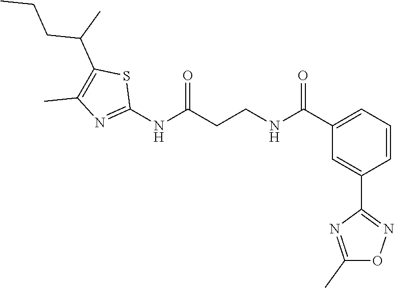 [SMILES: CCCC(C)c1sc(NC(=O)CCNC(=O)c2cccc(-c3noc(C)n3)c2)nc1C]

Example 129.1.: A mixture of 4-methylheptan-2-one (100.00 mg, 0.7800 mmol), thiourea (118.74 mg, 1.5599 mmol) and molecular iodine (197.96 mg, 0.7800 mmol) was dissolved in EtOH (1.56 mL) and heated overnight at 80 0° C. The reaction mixture was diluted with EtOAc, washed with an aqueous solution of NaOH (1 M) and an aqueous solution of NaS₂05, dried over MgSO₄ and concentrated under reduced pressure. Purification by silica gel column chromatography (eluent: 20-40% EtOAc in cyclohexane) affored 4-methyl-5-(1-methylbutyl)thiazol-2-amine (20 mg, 14%, 0.1085 mmol) as yellow oil. HPLC/MS m/z: 185.1, [M+H]⁺, Rt (R): 0.92 min.

Example 129.2.: 3-(5-methyl-1,2,4-oxadiazol-3-yl)-N-(3-((4-methyl-5-(pentan-2-yl)thiazol-2-yl)amino)-3-oxopropyl)benzamide was prepared using 4-methyl-5-(1-methylbutyl)thiazol-2-amine (12 mg, 28%, 0.0272 mmol) as a brown powder in an analogous procedure to 0. HPLC/MS m/z: 442.2, [M+H]⁺, Rt (R): 1.49 min. ¹H NMR (500 MHz, Chloroform-d) δ 8.41 (t, J=1.8 Hz, 1H), 8.17 (dt, J=7.7, 1.4 Hz, 1H), 7.93 (dt, J=8.0, 1.5 Hz, 1H), 7.54 (t, J=7.8 Hz, 1H), 7.13-7.07 (m, 1H), 3.89-3.81 (m, 2H), 3.01-2.93 (m, 1H), 2.84-2.79 (m, 2H), 2.66 (s, 3H), 2.21 (s, 3H), 1.62-1.46 (m, 2H), 1.31-1.19 (m, 5H), 0.87 (t, J=7.3 Hz, 3H) (1×NH not observed).

### Example 130: 4-Acetamidobutyl 4-methyl-2-(3-(3-(5-methyl-1,2,4-oxadiazol-3-yl)benzamido)propanamido)thiazole-5-carboxylate

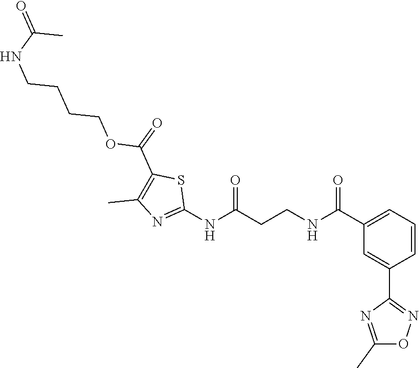 [SMILES: CC(=O)NCCCCOC(=O)c1sc(NC(=O)CCNC(=O)c2cccc(-c3noc(C)n3)c2)nc1C]

Acetyl acetate (6.58 uL, 0.0696 mmol) and 4-aminobutyl 4-methyl-2-[3-[[3-(5-methyl-1,2,4-oxadiazol-3-yl)benzoyl]amino]propanoylamino]thiazole-5-carboxylate hydrochloride [0](28.00 mg, 0.0535 mmol) were dissolved in dry THF (0.50 mL) at rt. TEA (22.39 uL, 0.1606 mmol) was added and the reaction mixture was stirred at rt for 1 h. The solvent was removed under reduced pressure and the residue was dissolved in DMSO (0.5 mL+0.3 mL) and purified by reverse phase column chromatography (eluent: 30-100% MeOH/H₂O+0.1% formic acid) to give 4-acetamidobutyl 4-methyl-2-(3-(3-(5-methyl-1,2,4-oxadiazol-3-yl)benzamido)propanamido)thiazole-5-carboxylate (20 mg, 71%, 0.0378 mmol) as a colorless powder. HPLC/MS m/z: 529.2, [M+H]⁺, Rt (R): 1.23 min. ¹H NMR (500 MHz, DMSO-d₆) δ 12.50 (s, 1H), 8.86 (t, J=5.6 Hz, 1H), 8.46 (t, J=1.8 Hz, 1H), 8.13 (dt, J=7.7, 1.4 Hz, 1H), 8.02 (dt, J=8.0, 1.4 Hz, 1H), 7.84 (t, J=5.6 Hz, 1H), 7.65 (t, J=7.8 Hz, 1H), 4.19 (t, J=6.5 Hz, 2H), 3.64-3.56 (m, 2H), 3.09-3.01 (m, 2H), 2.78 (t, J=6.8 Hz, 2H), 2.68 (s, 3H), 2.53 (s, 3H), 1.78 (s, 3H), 1.69-1.60 (m, 2H), 1.52-1.44 (m, 2H).

### Example 131: Propyl-1-methyl-3-(3-((7-(5-methyl-1,2,4-oxadiazol-3-yl)isoquinolin-1-yl)amino)propanamido)-1 H-pyrazole-5-carboxylate formate

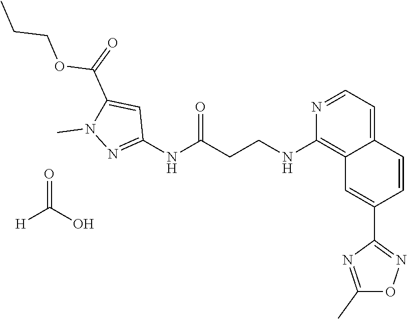

3-[[7-(5-methyl-1,2,4-oxadiazol-3-yl)-1-isoquinolyl]amino]propanoic acid [0](16.05 mg, 0.0538 mmol), HOBt (14.54 mg, 0.1076 mmol), EDC.HCl (20.63 mg, 0.1076 mmol) and propyl 5-amino-2-methyl-pyrazole-3-carboxylate (11.83 mg, 0.0646 mmol) were dissolved in dry DMF (0.29 mL). The reaction mixture was warmed to 60° C. and stirred overnight. Purified by reverse phase column chromatography (eluent: 30-100% MeOH/H₂O+0.1% formic acid) to afford propyl 1-methyl-3-(3-((7-(5-methyl-1,2,4-oxadiazol-3-yl)isoquinolin-1-yl)amino)propanamido)-1 H-pyrazole-5-carboxylate formate (11 mg, 44%, 0.0237 mmol) as a yellow powder. HPLC/MS m/z: 464.2, [M+H]⁺, Rt (R): 1.18 min. ¹H NMR (500 MHz, DMSO-d₆) δ 10.69 (s, 1H), 8.90-8.87 (m, 1H), 8.23-8.18 (m, 1H), 8.14 (dd, J=8.5, 1.5 Hz, 1H), 8.00-7.94 (m, 2H), 7.84 (d, J=8.5 Hz, 1H), 7.06 (s, 1H), 6.96 (d, J=5.7 Hz, 1H), 4.20 (t, J=6.5 Hz, 2H), 3.98 (s, 3H), 3.80-3.73 (m, 2H), 2.74 (t, J=7.0 Hz, 2H), 2.70 (s, 3H), 1.74-1.66 (m, 2H), 0.95 (t, J=7.4 Hz, 3H).

The following examples were prepared by an analogous procedure:

### Example 132: Tert-butyl-4-methyl-2-(3-(3-(5-methyl-1,2,4-oxadiazol-3-yl)benzamido)propanamido)thiazole-5-carboxylate

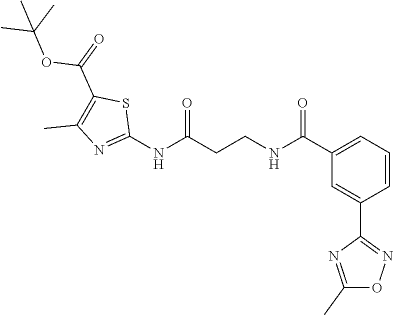 [SMILES: Cc1nc(-c2cccc(C(=O)NCCC(=O)Nc3nc(C)c(C(=O)OC(C)(C)C)s3)c2)no1]

13 mg (36%), slightly yellow powder. HPLC/MS m/z: 472.17, [M+H]⁺, Rt (Z): 2.80 min. ¹H NMR (600 MHz, DMSO-d₆) δ 12.41 (s, 1H), 8.86 (t, J=5.5 Hz, 1H), 8.47 (t, J=1.8 Hz, 1H), 8.14 (dt, J=7.7, 1.4 Hz, 1H), 8.04 (ddd, J=7.9, 1.8, 1.2 Hz, 1H), 7.67 (t, J=7.8 Hz, 1H), 3.63-3.58 (m, 2H), 2.79 (t, J=6.8 Hz, 2H), 2.70 (s, 3H), 2.51 (d, J=1.0 Hz, 3H), 1.52 (s, 9H).

### Example 133: Ethyl-3-methyl-5-[3-[[3-(5-methyl-1,2,4-oxadiazol-3-yl)benzoyl]amino]propanoylamino]thiophene-2-carboxylate

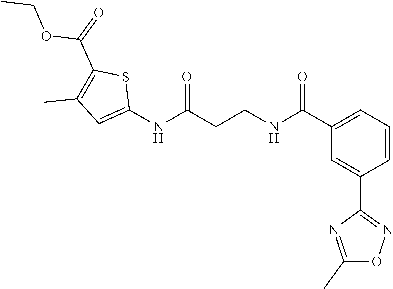 [SMILES: CCOC(=O)c1sc(NC(=O)CCNC(=O)c2cccc(-c3noc(C)n3)c2)cc1C]

6.8 mg (8%), amorphous colorless solid. HPLC/MS m/z: 443.14 [M+H]⁺, Rt (R): 1.32 min. ¹H NMR (500 MHz, DMSO-d₆) δ 11.58 (s, 1H), 8.88 (t, J=5.6 Hz, 1H), 8.46 (d, J=1.8 Hz, 1H), 8.13 (dt, J=7.9, 1.4 Hz, 1H), 8.03 (dt, J=7.7, 1.4 Hz, 1H), 7.65 (t, J=7.8 Hz, 1H), 6.52 (s, 1H), 4.20 (q, J=7.1 Hz, 2H), 3.58 (q, J=6.5 Hz, 2H), 2.71 (t, J=6.9 Hz, 2H), 2.68 (s, 3H), 2.40 (s, 3H), 1.26 (t, J=7.1 Hz, 3H).

### Example 134: Ethyl-4-methyl-2-(3-(3-(5-methyl-1,2,4-oxadiazol-3-yl)benzamido)-N-propylpropanamido)thiazole-5-carboxylate

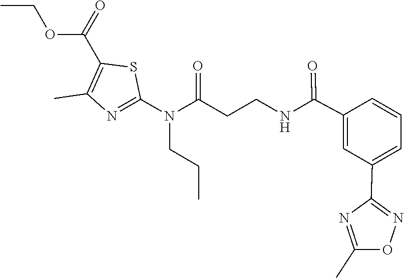 [SMILES: CCCN(C(=O)CCNC(=O)c1cccc(-c2noc(C)n2)c1)c1nc(C)c(C(=O)OCC)s1]

Example 134.1.: Sodium triacetoxyborohydride (227.61 mg, 1.0739 mmol) was added to a solution of ethyl 2-amino-4-methyl-thiazole-5-carboxylate (100.00 mg, 0.5370 mmol) and propanal (0.12 mL, 1.6109 mmol) in DCE (3.58 mL). Acetic acid (0.05 mL, 0.8055 mmol) was added, and the solution was heated under microwave irradiation for 30 min at 80 0° C. The reaction mixture was quenched with a saturated hydrogenocarbonate solution (20 mL) and extracted with DCM (20 mL). The organic layers were washed with brine (20 mL), dried over MgSO₄ and concentrated under reduced pressure to afford ethyl 4-methyl-2-(propylamino)thiazole-5-carboxylate (129 mg, 105%, 0.5650 mmol) as a yellow oil which was used in the next step without further purification. HPLC/MS m/z: 229.10, [M+H]⁺, Rt (P): 1.38 min.

Example 134.2.: Using ethyl 4-methyl-2-(propylamino)thiazole-5-carboxylate in a procedure analogous to 0 afforded ethyl 4-methyl-2-(3-(3-(5-methyl-1,2,4-oxadiazol-3-yl)benzamido)-N-propylpropanamido)thiazole-5-carboxylate (15 mg, 11%, 0.0309 mmol) as a colorless solid. HPLC/MS m/z: 486.18, [M+H]⁺, Rt (S): 3.16 min. ¹H NMR (500 MHz, DMSO-d₆) δ 8.81 (t, J=5.4 Hz, 1H), 8.47 (d, J=1.8 Hz, 1H), 8.13 (dt, J=7.7, 1.4 Hz, 1H), 8.04 (dt, J=7.9, 1.5 Hz, 1H), 7.66 (t, J=7.8 Hz, 1H), 4.24 (q, J=7.1 Hz, 2H), 4.11 (t, J=7.9 Hz, 2H), 3.64 (q, J=6.5 Hz, 2H), 3.09 (t, J=6.8 Hz, 2H), 2.68 (s, 3H), 2.56 (s, 3H), 1.72 (sext, J=7.5 Hz, 2H), 1.28 (t, J=7.1 Hz, 3H), 0.92 (t, J=7.4 Hz, 3H).

### Example 135: Ethyl-3-(3-((7-(5-methyl-1,2,4-oxadiazol-3-yl)isoquinolin-1-yl)amino)propanamido)-1 H-pyrazole-5-carboxylate

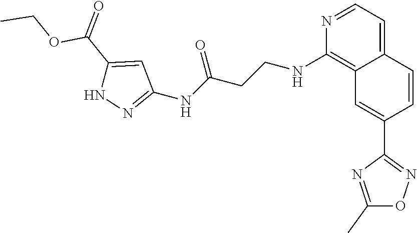 [SMILES: CCOC(=O)c1cc(NC(=O)CCNc2nccc3ccc(-c4noc(C)n4)cc23)n[nH]1]

To 3-[[7-(5-methyl-1,2,4-oxadiazol-3-yl)-1-isoquinolyl]amino]propanoic acid [0](30.00 mg, 0.1006 mmol) and PyBrop (93.77 mg, 0.2011 mmol) was added Ethyl 5-amino-1 h-pyrazole-3-carboxylate (18.72 mg, 0.1207 mmol) dissolved in anhydrous DMF (0.63 mL). To the solution was added DIPEA (70.07 uL, 0.4023 mmol) and stirred at rt for 3 d. Purified by reverse phase column chromatography (eluent: 20-100% MeOH in water (+0.1% formic acid modifier in both)) followed by ion-exchange column SCX-II, eluting with 2M NH₃ in MeOH, afforded ethyl 3-(3-((7-(5-methyl-1,2,4-oxadiazol-3-yl)isoquinolin-1-yl)amino)propanamido)-1 H-pyrazole-5-carboxylate (13 mg, 30%, 0.0299 mmol). HPLC/MS m/z: 436.2 [M+H]⁺, Rt (U): 2.12 min. ¹H NMR (500 MHz, DMSO-d₆) δ 13.49 (s, 1H), 10.68 (s, 1H), 8.91-8.86 (m, 1H), 8.14 (dd, J=8.5, 1.6 Hz, 1H), 7.98 (d, J=8.3 Hz, 2H), 7.85 (d, J=8.5 Hz, 1H), 7.03 (s, 1H), 6.96 (d, J=5.7 Hz, 1H), 4.29 (q, J=7.1 Hz, 2H), 3.77 (q, J=6.9, 6.5 Hz, 2H), 2.75 (t, J=7.0 Hz, 2H), 2.70 (s, 3H), 1.30 (t, J=7.1 Hz, 3H).

### Example 136: 6-Hydroxyhexyl 4-methyl-2-(3-(3-(5-methyl-1,2,4-oxadiazol-3-yl)-5-(trifluoromethyl)benzamido)propanamido)thiazole-5-carboxylate

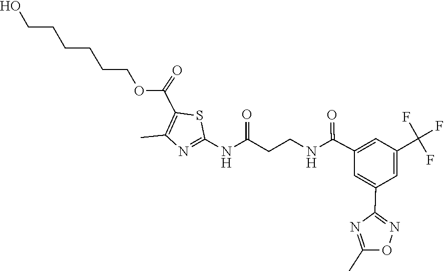 [SMILES: Cc1nc(-c2cc(C(=O)NCCC(=O)Nc3nc(C)c(C(=O)OCCCCCCO)s3)cc(C(F)(F)F)c2)no1]

Example 136.1.: Through a mixture of methyl 3-bromo-5-(trifluoromethyl)benzoate (5.50 g, 19.432 mmol) and zinc cyanide (2.97 g, 25.261 mmol) in DMF (97.16 mL) nitrogen was bubbled for 15 min. Palladium tetrakis(triphenylphosphine) (1347.28 mg, 1.1659 mmol) was added, purged with nitrogen for 5 min and heated at 100° C. for 3 h. Added to brine (800 mL), extracted with diethyl ether (2×100 mL), washed combined organic layers with brine (2×250 mL). Dried over magnesium sulfate. Purification by silica gel column chromatography (Eluent: 0-10% EtOAc in cyclohexane) to afford methyl 3-cyano-5-(trifluoromethyl)benzoate (4.03 g, 90%, 17.564 mmol). ¹H NMR (500 MHz, Chloroform-d) δ 8.53-8.48 (m, 2H), 8.09 (td, J=1.6, 0.8 Hz, 1H), 4.01 (s, 3H).

Example 136.2.: Methyl 3-cyano-5-(trifluoromethyl)benzoate (533.60 mg, 2.3285 mmol) and TEA (0.32 mL, 2.3285 mmol) were heated at 80° C. in [bmim]OAc (2.33 mL) and hydroxylamine hydrochloride (323.62 mg, 4.657 mmol) was added. The solution was stirred for 15 min at 80 ° C. The mixture was cooled down to rt and water (50 mL) was added. Residue was extracted with EtOAc (3×25 mL). Organic layers were combined, washed with brine (25 mL), dried over MgSO₄ and concentrated under reduced pressure. Purified by reverse phase column chromatography (eluent: 10-100% MeOH in water (+0.1% formic acid in both)) to afford methyl 3-[(Z)-N′-hydroxycarbamimidoyl]-5-(trifluoromethyl)benzoate (390 mg, 64%, 1.4875 mmol) as a colorless solid. HPLC/MS m/z: 263.1, [M+H]⁺, Rt (P): 0.95 min.

Example 136.3.: Acetic acid (0.08 mL, 1.4722 mmol), methyl 3-(N-hydroxycarbamimidoyl)-5-(trifluoromethyl)benzoate (386.00 mg, 1.4722 mmol) and EDC.HCl (310.45 mg, 1.6194 mmol) were dissolved in MeCN (4.82 mL) and THE (4.82 mL) under nitrogen atmosphere. The solution was stirred overnight at rt. There was still starting material. EDC.HCl (0.2 eq) and acetic acid (0.2 eq) were added, and the solution was stirred at rt for further 3 h. Volatiles were removed under reduced pressure to afford methyl 3-(N-acetoxycarbamimidoyl)-5-(trifluoromethyl)benzoate (450 mg, 100%, 1.4792 mmol) as a colourless oil which was used in the next step without further purification. HPLC/MS m/z: 305.1, [M+H]⁺, Rt (P): 1.32 min.

Example 136.4.: To a solution of methyl 3-(N-acetoxycarbamimidoyl)-5-(trifluoromethyl)benzoate (447.87 mg, 1.4722 mmol) in DMSO (1.47 mL) was added potassium hydroxide (272.60 mg, 4.8583 mmol). The solution was stirred at rt for 60 min. Water (1 mL) was added in order to carry out the saponification. The solution was then stirred at rt for further 1 h. LC/MS showed complete conversion into the expected carboxylic acid. Water (20 mL) was added, and the aqueous layer was washed with EtOAc (20 mL). Aqueous layer was acidified (until pH around 1) by adding few drops of fuming HCl (37%). Residue was extracted with EtOAc (3×20 mL). Organic layers were combined, washed with brine (25 mL), dried over magnesium sulfate, and concentrated under reduced pressure to afford 3-(5-methyl-1,2,4-oxadiazol-3-yl)-5-(trifluoromethyl)benzoic acid (360 mg, 90%, 1.3227 mmol) as a colorless solid. HPLC/MS m/z: 273.0 [M+H]⁺, Rt (P): 1.49 min.

Example 136.5.: 3-[[3-(5-methyl-1,2,4-oxadiazol-3-yl)-5-(trifluoromethyl)benzoyl]amino]propanoic acid was prepared using an analogous procedure to 0 using 3-(5-methyl-1,2,4-oxadiazol-3-yl)-5-(trifluoromethyl)benzoic acid. HPLC/MS m/z: 344.1, [M+H]⁺, Rt (R): 1.21 min.

Example 136.6.: Procedure analogous to 0 using hexane-1,6-diol afforded 6-hydroxyhexyl 4-methyl-2-(3-(3-(5-methyl-1,2,4-oxadiazol-3-yl)-5-(trifluoromethyl)benzamido)propanamido)thiazole-5-carboxylate (43 mg, 80%, 0.0737 mmol) as a cream-coloured powder. HPLC/MS m/z: 584.2, [M+H]⁺, Rt (R): 1.46 min. ¹H NMR (500 MHz, Methanol-d₄) δ 8.75-8.70 (m, 1H), 8.46-8.40 (m, 1H), 8.31-8.25 (m, 1H), 4.25 (t, J=6.5 Hz, 2H), 3.79 (t, J=6.6 Hz, 2H), 3.56 (t, J=6.6 Hz, 2H), 2.87 (t, J=6.6 Hz, 2H), 2.68 (s, 3H), 2.55 (s, 3H), 1.78-1.70 (m, 2H), 1.61-1.52 (m, 2H), 1.51-1.40 (m, 4H).

### Example 137: 5-Butyl-4-methyl-N-(2-((7-(5-methyl-1,2,4-oxadiazol-3-yl)isoquinolin-1-yl)amino)ethyl)thiazole-2-carboxamide formate

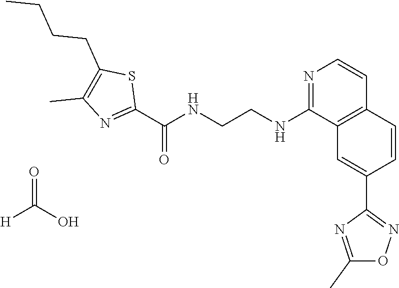 [SMILES: O=CO ; CCCCc1sc(C(=O)NCCNc2nccc3ccc(-c4noc(C)n4)cc23)nc1C]

To a stirred solution containing 5-butyl-4-methyl-thiazole-2-carboxylic acid (16.82 mg, 0.0844 mmol), HATU (48.13 mg, 0.1266 mmol) and DIPEA (29.40 uL, 0.1688 mmol) in DMF (0.30 mL) was added N′-[7-(5-methyl-1,2,4-oxadiazol-3-yl)-1-isoquinolyl]ethane-1,2-diamine [0](25.00 mg, 0.0928 mmol). The resulting solution was stirred at rt overnight. Purified by reverse phase column chromatography (eluent: 40-90% MeOH/H₂O+0.1% formic acid) afforded 5-butyl-4-methyl-N-(2-((7-(5-methyl-1,2,4-oxadiazol-3-yl)isoquinolin-1-yl)amino)ethyl)thiazole-2-carboxamide formate (8 mg, 19%, 0.0178 mmol) a cream-coloured powder. HPLC/MS m/z: 451.2, [M+H]⁺, Rt (T): 1.28 min. ¹H NMR (600 MHz, Methanol-d₄) δ 8.79 (s, 1H), 8.35-8.22 (m, 1H), 8.17 (dd, J=8.5, 1.8 Hz, 1H), 7.86 (d, J=6.1 Hz, 1H), 7.76 (d, J=8.4 Hz, 1H), 6.98 (d, J=6.0 Hz, 1H), 3.84-3.76 (m, 2H), 3.76-3.69 (m, 2H), 2.75 (t, J=7.5 Hz, 2H), 2.65 (s, 3H), 2.32 (s, 3H), 1.62-1.52 (m, 2H), 1.40-1.30 (m, 2H), 0.92 (t, J=7.4 Hz, 3H).

### Example 138: Cyclopentyl-1-methyl-3-(3-((7-(5-methyl-1,2,4-oxadiazol-3-yl)isoquinolin-1-yl)amino)propanamido)-1 H-pyrazole-5-carboxylate

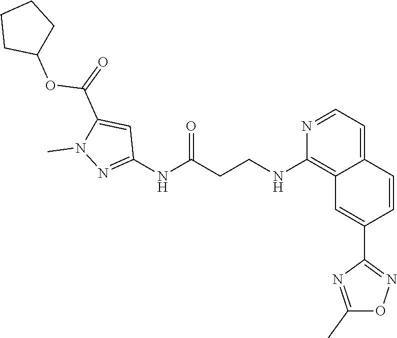 [SMILES: Cc1nc(-c2ccc3ccnc(NCCC(=O)Nc4cc(C(=O)OC5CCCC5)n(C)n4)c3c2)no1]

Example 138.1.: To a solution of 3-amino-1-methyl-1 H-pyrazole-5-carboxylic acid (50.00 mg, 0.3543 mmol) and DMAP (4.33 mg, 0.0354 mmol) in DCM (3.31 mL) was successively added 1 M DCC (0.43 mL, 0.4251 mmol) and cyclopentanol (0.32 mL, 3.5428 mmol). The resulting solution was stirred at rt overnight. LCMS showed complete conversion. Volatiles were removed under vacuum and the crude product was purified by reverse phase chromatography. Residue was dissolved in DMSO (0.8 mL, RBF washed with 0.3 mL DMSO) and loaded onto a 12 g C18 SNAP Ultra column. Column was eluted with 20-70% MeOH in water (+0.1% formic acid). Tubes 7 to 10 were passed through a SCX-II cartridge (5 g). Column was eluted with MeOH followed by 2M ammonia solution in MeOH. Basic fraction was concentrated under reduced pressure to afford cyclopentyl 5-amino-2-methyl-pyrazole-3-carboxylate (35 mg, 47%, 0.1673 mmol) as a colourless oil. HPLC/MS m/z: 210.13, [M+H]⁺, Rt (T): 1.22 min.

Example 138.2.: Cyclopentyl 5-amino-2-methyl-pyrazole-3-carboxylate used in an analogous procedure to 0 to afford cyclopentyl 2-methyl-5-[3-[[7-(5-methyl-1,2,4-oxadiazol-3-yl)-1-isoquinolyl]amino]propanoylamino]pyrazole-3-carboxylate (8.8 mg, 27%, 0.0180 mmol) as a colorless solid. HPLC/MS m/z: 490.22, [M+H]⁺, Rt (U): 2.67 min. ¹H NMR (600 MHz, DMSO-d₆) δ 10.67 (s, 1H), 8.91-8.85 (m, 1H), 8.15 (dd, J=8.5, 1.5 Hz, 1H), 7.99 (d, J=5.7 Hz, 1H), 7.96 (t, J=5.5 Hz, 1H), 7.85 (d, J=8.5 Hz, 1H), 7.02 (s, 1H), 6.97 (d, J=5.7 Hz, 1H), 5.29 (dq, J=6.1, 3.0 Hz, 1H), 3.97 (s, 3H), 3.77 (td, J=7.0, 5.2 Hz, 2H), 2.74 (t, J=7.0 Hz, 2H), 2.71 (s, 3H), 1.96-1.86 (m, 2H), 1.74 (dddd, J=20.9, 11.9, 6.5, 3.5 Hz, 4H), 1.65-1.56 (m, 2H).

### Example 139: 3-{[3-(5-methyl-1,2,4-oxadiazol-3-yl)phenyl]formamido}-N-[4-methyl-5-(trifluoromethyl)-1,3-thiazol-2-yl]propenamide

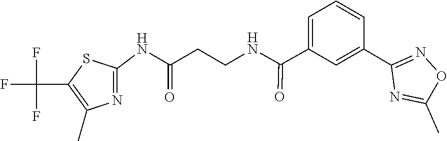 [SMILES: Cc1nc(-c2cccc(C(=O)NCCC(=O)Nc3nc(C)c(C(F)(F)F)s3)c2)no1]

Example 139.1.: 4-Methyl-5-(trifluoromethyl)-1,3-thiazol-2-amine (142.5 mg, 0.782 mmol), Boc-p-alanine (177.6 mg, 0.939 mmol) and HATU (446.0 mg, 1.173 mmol) were dissolved in DMF (2.0 mL). N-Ethyldiisopropylamine (202.1 mg, 1.564 mmol) was added, and the brown solution was stirred at rt for 3 h. The reaction mixture was diluted with ethyl acetate (30 mL), washed with water and brine, dried with sodium sulfate, filtered, and evaporated to dryness. The oily residue was purified by flash chromatography to yield 199 mg light brown solid, which was suspended in dioxane (3.0 mL), treated with HCl in 1,4-dioxane (4 M, 3.2 mL) and stirred at rt for 2 h. The reaction mixture was evaporated to dryness to give 184 mg of 3-amino-N-[4-methyl-5-(trifluoromethyl)-1,3-thiazol-2-yl]propanamide dihydrochloride as a yellow solid. HPLC/MS m/z: 254.1 [M+H]⁺, Rt (M): 1.18 min.

Example 139.2.: 3-Amino-N-[4-methyl-5-(trifluoromethyl)-1,3-thiazol-2-yl]propanamide dihydrochloride (184.0 mg, 0.564 mmol), 3-(5-methyl-1,2,4-oxadiazol-3-yl)benzoic acid (138.2 mg, 0.677 mmol) and [Dimethylamino-([1,2,3]triazolo[4,5-b]pyridin-3-yloxy)-methylene]-dimethyl-ammonium; hexafluoro phosphate (257.4 mg, 0.677 mmol) were placed in a vial and suspended in DMF (2.5 mL). N-Ethyldiisopropylamine (310.0 μL, 1.805 mmol) was added and the clear brown solution was stirred at rt overnight. The reaction mixture was diluted with ethyl acetate (40 mL), washed with water and brine, dried with sodium sulfate, filtered, and evaporated to dryness. The oily residue was purified by flash chromatography, and the resulting solid was triturated with acetonitrile, filtered by suction and washed with a small amount of acetonitrile and tert-butyl methyl ether, and dried under vacuum at 60° C. overnight to give 167 mg (67%) of 3-{[3-(5-methyl-1,2,4-oxadiazol-3-yl)phenyl]formamido}-N-[4-methyl-5-(trifluoromethyl)-1,3-thiazol-2-yl]propenamide as a colorless solid. HPLC/MS m/z: 440.1 [M+H]⁺, Rt (M): 1.60 min. ¹H NMR (500 MHz, DMSO-d₆): δ 12.62 (s, 1H), 8.83 (t, J=5.5 Hz, 1H), 8.45 (t, J=1.7 Hz, 1H), 8.12 (dt, J=7.7, 1.4 Hz, 1H), 8.03-8.00 (m, 1H), 7.65 (t, J=7.8 Hz, 1H), 3.61 (q, J=6.6 Hz, 2H), 2.80 (t, J=6.8 Hz, 2H), 2.68 (s, 3H), 2.40-2.37 (m, 3H).

### Example 140: Ethyl-2-((2-(3-(5-ethyl-1,2,4-oxadiazol-3-yl)benzamido)ethyl)carbamoyl)-4-methylthiazole-5-carboxylate

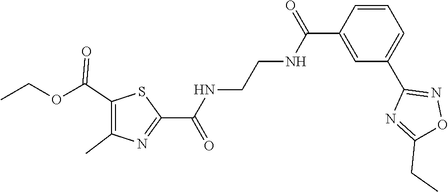 [SMILES: CCOC(=O)c1sc(C(=O)NCCNC(=O)c2cccc(-c3noc(CC)n3)c2)nc1C]

Example 140.1.: A solution of chloro(isopropyl)magnesium; chlorolithium (3.60 mL, 4.6779 mmol) was added to a solution of ethyl 2-bromo-4-methyl-thiazole-5-carboxylate (0.90 g, 3.5984 mmol) in dry THF (5 mL) at −78 0° C. The reaction mixture was stirred for 10 min at −78° C. before the addition of morpholine-4-carbaldehyde (0.90 mL, 8.996 mmol). After 25 min the reaction mixture was quenched with NH₄Cl (10 mL). The aqueous layer was extracted with EtOAc (2×10 mL), dried over MgSO₄ and concentrated under reduced vacuum. Purified by silica gel column chromatography (Eluent: 0-20% EtOAc in cyclohexane) to afford ethyl 2-formyl-4-methyl-thiazole-5-carboxylate (495 mg, 69%, 2.4846 mmol) as a colourless oil. HPLC/MS m/z: 200.0, [M+H]⁺, Rt (Q): 2.33 min.

Example 140.2.: 2-Methylbut-2-ene (7.82 mL, 73.784 mmol) was added to a solution of ethyl 2-formyl-4-methyl-thiazole-5-carboxylate (490.00 mg, 2.4595 mmol) in THF (8.20 mL) and t-BuOH (8.20 mL) at rt. After 5 min, a solution of sodium dihydrogen phosphate (885.26 mg, 7.3784 mmol) and sodium chlorite (734.03 mg, 8.1162 mmol) in H₂O (4 mL) was added dropwise. The reaction mixture was stirred for 1.5 h at rt. The reaction mixture was quenched with a satured solution of Na₂S₂O₃. The aqueous layer was extracted with EtOAc, then acidified to pH=1-2 with a solution of 2N HCl and extracted again with EtOAc. The organic layer was dried over MgSO₄ and concentrated under reduced pressure to afford 5-ethoxycarbonyl-4-methyl-thiazole-2-carboxylic acid (382 mg, 72%, 1.7748 mmol) which was used without further purification.

Example 140.3.: tert-Butyl N-(2-aminoethyl)carbamate (0.09 mL, 0.5766 mmol) was added to a solution of 5-ethoxycarbonyl-4-methyl-thiazole-2-carboxylic acid (73.00 mg, 0.3392 mmol), 3-(ethyliminomethyleneamino)-N,N-dimethyl-propan-1-amine, HCl (135.00 mg, 0.8696 mmol) and 1-hydroxybenzotriazole (117.00 mg, 0.8659 mmol) in dry DMF at rt. The reaction mixture was stirred overnight at rt. The mixture was diluted with EtOAc (10 mL) and washed with a saturated solution of NaHCO₃ (2×5 mL) and water (2×5 mL), dried over MgSO₄ and concentrated in vacuo. Purified by silica gel column chromatography (Eluent: 5-50% EtOAc in cyclohexane) to give ethyl 2-[2-(tert-butoxycarbonylamino)ethylcarbamoyl]-4-methyl-thiazole-5-carboxylate (50 mg, 41%, 0.1399 mmol) as a colorless powder. HPLC/MS m/z: 358.1, [M+H]⁺, Rt (R): 2.84 min.

Example 140.4.: HCl in dioxane (0.59 mL, 2.3501 mmol) was added to a solution of ethyl 2-[2-(tert-butoxycarbonylamino)ethylcarbamoyl]-4-methyl-thiazole-5-carboxylate (42.00 mg, 0.1175 mmol) in dry dioxane (0.59 mL) at rt. The reaction mixture was stirred overnight at rt. The solvent was removed under reduced pressure to give ethyl 2-(2-aminoethylcarbamoyl)-4-methyl-thiazole-5-carboxylate as a colorless solid. No further purification.

Example 140.5.: Ethyl 2-(2-aminoethylcarbamoyl)-4-methyl-thiazole-5-carboxylate (28.00 mg, 0.1088 mmol), 3-(5-ethyl-1,2,4-oxadiazol-3-yl)benzoic acid (28.49 mg, 0.1306 mmol), HOBt (29.41 mg, 0.2176 mmol) and EDC (33.79 mg, 0.2176 mmol) were dissolved in dry DMF (0.54 mL) at rt. The reaction mixture was stirred overnight at rt. The reaction mixture was diluted with EtOAc and washed with NaHCO₃ (2×8 mL) and water (8 mL), dried over MgSO₄, concentrated under reduced pressure and dissolved in DMSO (0.5 mL+0.3 mL). Purification by reverse phase column chromatography (eluent: 30-100% MeOH/H₂O+0.1% formic acid) afforded ethyl 2-((2-(3-(5-ethyl-1,2,4-oxadiazol-3-yl)benzamido)ethyl)carbamoyl)-4-methylthiazole-5-carboxylate (30 mg, 57%, 0.0623 mmol). HPLC/MS m/z: 458.1, [M+H]⁺, Rt (S): 2.96 min. ¹H NMR (500 MHz, DMSO-d₆) δ 9.17-9.09 (m, 1H), 8.85-8.78 (m, 1H), 8.45 (t, J=1.8 Hz, 1H), 8.14 (dt, J=7.8, 1.4 Hz, 1H), 8.02 (dt, J=8.0, 1.4 Hz, 1H), 7.66 (t, J=7.7 Hz, 1H), 4.30 (q, J=7.1 Hz, 2H), 3.53-3.45 (m, 4H), 3.08-2.99 (m, 2H), 2.69 (s, 3H), 1.36 (t, J=7.6 Hz, 3H), 1.30 (t, J=7.1 Hz, 3H).

The following examples were prepared analogously:

### Example 141: Ethyl-4-methyl-2-((2-(3-(5-methyl-1,2,4-oxadiazol-3-yl)benzamido)ethyl)carbamoyl)thiazole-5-carboxylate

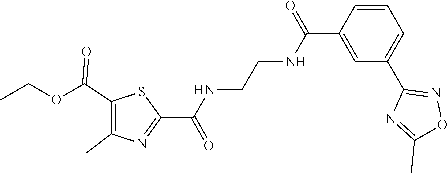 [SMILES: CCOC(=O)c1sc(C(=O)NCCNC(=O)c2cccc(-c3noc(C)n3)c2)nc1C]

48 mg (51%), colorless solid. HPLC/MS m/z: 444.1, [M+H]⁺, Rt (R): 1.32 min. ¹H NMR (500 MHz, DMSO-d₆) δ 9.16-9.10 (m, 1H), 8.83-8.79 (m, 1H), 8.45 (t, J=1.7 Hz, 1H), 8.12 (dt, J=7.8, 1.4 Hz, 1H), 8.02 (dt, J=7.9, 1.4 Hz, 1H), 7.65 (t, J=7.8 Hz, 1H), 4.30 (q, J=7.1 Hz, 2H), 3.52-3.43 (m, 4H), 2.69 (s, 3H), 2.68 (s, 3H), 1.30 (t, J=7.1 Hz, 3H).

### Example 142: Ethyl-(R)-4-methyl-2-(3-(3-(5-methyl-1,2,4-oxadiazol-3-yl)benzamido)butanamido)thiazole-5-carboxylate

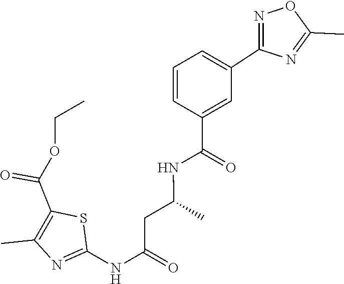

Example 142.1.: To tert-Butyl (3R)-3-aminobutanoate (155.97 mg, 0.9795 mmol) was added anhydrous DMF (4.90 mL), 3-(5-methyl-1,2,4-oxadiazol-3-yl)benzoic acid (200.00 mg, 0.9795 mmol) DIPEA (0.51 mL, 2.9386 mmol) and HATU (345.68 mg, 1.47 mmol). The reaction mixture was stirred at rt overnight. Water (70 mL) was added, and the mixture extracted with EtOAc (2×60 mL). The combined organics were washed with saturated NaHCO₃, brine, dried over MgSO₄ before concentrating in vacuo to give tert-butyl (3R)-3-[[3-(5-methyl-1,2,4-oxadiazol-3-yl)benzoyl]amino]butanoate (335 mg, 99%, 0.9699 mmol) as a clear viscous oil. Used without further purification. HPLC/MS m/z: 368.1 [M+Na]⁺, Rt (P): 1.45 min.

Example 142.2.: To tert-butyl (3R)-3-[[3-(5-methyl-1,2,4-oxadiazol-3-yl)benzoyl]amino]butanoate (167.00 mg, 0.4835 mmol) in DCM (1.50 mL) was added TFA (0.37 mL, 4.8351 mmol) and the mixture stirred for 2 h. The volatiles were removed in vacuo. Toluene was added and then evaporated to help remove traces of high boiling TFA and by products. High vacuum+45° C. water bath used to leave (3R)-3-[[3-(5-methyl-1,2,4-oxadiazol-3-yl)benzoyl]amino]butanoic acid (142 mg, 102%, 0.4909 mmol) as colorless solid. No further purification. HPLC/MS m/z: 458.1 [M+H]⁺, Rt (P): 1.51 min.

Example 142.3.: A 50 mL RBF was charged with (3R)-3-[[3-(5-methyl-1,2,4-oxadiazol-3-yl)benzoyl]amino]butanoic acid (140.00 mg, 0.4839 mmol), ethyl 2-amino-4-methyl-thiazole-5-carboxylate (90.12 mg, 0.4839 mmol), HOBt (130.78 mg, 0.9679 mmol) and dry DMF (2.42 mL). To this mixture was added EDC (185.54 mg, 0.9679 mmol). A condensor, needle and empty balloon added, and stirred for 18 h at 60° C. LCMS showed the desired product present. Water (50 mL) was added. This was extracted with EtOAc (2×35 mL), these organics were combined, washed with saturated NaHCO₃ (25 mL), brine (25 mL), dried over MgSO₄ and concentrated in vacuo to give a colorless solid. Purification by reverse phase column chromatography (elutent: 20-80% MeOH in water (+0.1% formic acid modifier in both)) afforded ethyl (R)-4-methyl-2-(3-(3-(5-methyl-1,2,4-oxadiazol-3-yl)benzamido)butanamido)thiazole-5-carboxylate (10 mg, 5%, 0.0219 mmol). HPLC/MS m/z: 458.1 [M+H]⁺, Rt (P): 1.51 min. ¹H NMR (500 MHz, DMSO-d₆) δ 12.55 (s, 1H), 8.64 (d, J=8.0 Hz, 1H), 8.44 (t, J=1.8 Hz, 1H), 8.12 (dt, J=7.8, 1.4 Hz, 1H), 8.01 (dt, J=7.9, 1.4 Hz, 1H), 7.65 (t, J=7.8 Hz, 1H), 4.57-4.44 (m, 1H), 4.22 (q, J=7.1 Hz, 2H), 2.78 (dd, J=14.9, 7.2 Hz, 1H), 2.72 (dd, J=14.9, 6.8 Hz, 1H), 2.69 (s, 3H), 2.53 (s, 3H), 1.26 (t, J=7.1 Hz, 3H), 1.23 (d, J=6.7 Hz, 3H).

The following examples were prepared in an analogous procedure:

### Example 143: Ethyl-(S)-4-methyl-2-(3-(3-(5-methyl-1,2,4-oxadiazol-3-yl)benzamido)butanamido)thiazole-5-carboxylate

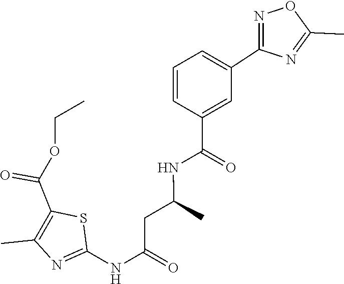

15 mg (10%). HPLC/MS m/z: 458.1 [M+H]⁺, Rt (S): 2.82 min. ¹H NMR (500 MHz, DMSO-d₆) δ 12.55 (s, 1H), 8.64 (d, J=8.0 Hz, 1H), 8.44 (dt, J=1.7, 1.0 Hz, 1H), 8.15-8.09 (m, 1H), 8.01 (ddd, J=7.8, 1.9, 1.2 Hz, 1H), 7.65 (dt, J=7.6, 0.6 Hz, 1H), 4.51 (dt, J=13.9, 7.0 Hz, 1H), 4.22 (q, J=7.1 Hz, 2H), 2.83-2.70 (m, 2H), 2.69 (s, 3H), 2.53 (s, 3H), 1.26 (t, J=7.1 Hz, 3H), 1.23 (d, J=6.7 Hz, 3H).

### Example 144: Propyl-2-(2-(4-(3-chloro-5-(5-methyl-1,2,4-oxadiazol-3-yl)benzamido)-1-methylpiperidin-4-yl)acetamido)-4-methylthiazole-5-carboxylate

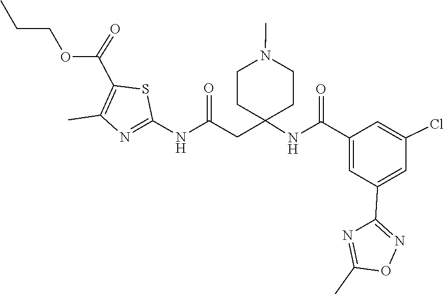 [SMILES: CCCOC(=O)c1sc(NC(=O)CC2(NC(=O)c3cc(Cl)cc(-c4noc(C)n4)c3)CCN(C)CC2)nc1C]

tert-Butyl 2-(4-amino-1-methyl-4-piperidyl)acetate and 3-chloro-5-(5-methyl-1,2,4-oxadiazol-3-yl)benzoic acid afforded propyl 2-(2-(4-(3-chloro-5-(5-methyl-1,2,4-oxadiazol-3-yl)benzamido)-1-methylpiperidin-4-yl)acetamido)-4-methylthiazole-5-carboxylate (7 mg, 20%, 0.0122 mmol) as a colorless solid. HPLC/MS m/z: 575.18, [M+H]⁺, Rt (S): 2.66 min. ¹H NMR (500 MHz, Methanol-d₄) δ 8.30 (t, J=1.5 Hz, 1H), 8.13 (dd, J=2.1, 1.5 Hz, 1H), 7.92 (t, J=1.8 Hz, 1H), 4.20 (t, J=6.5 Hz, 2H), 3.10 (s, 2H), 2.80-2.73 (m, 2H), 2.69 (s, 3H), 2.66-2.58 (m, 2H), 2.55 (s, 3H), 2.48-2.39 (m, 2H), 2.35 (s, 3H), 1.95-1.84 (m, 2H), 1.74 (dtd, J=13.8, 7.4, 6.5 Hz, 2H), 1.01 (t, J=7.4 Hz, 3H).

### Example 145: Ethyl-4-methyl-2-(2-methyl-3-(3-(5-methyl-1,2,4-oxadiazol-3-yl)benzamido)propanamido)thiazole-5-carboxylate

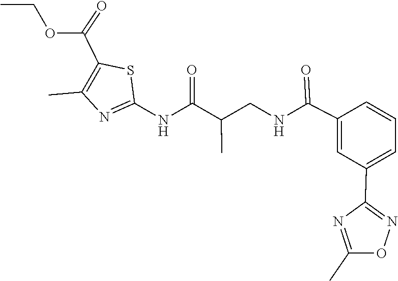 [SMILES: CCOC(=O)c1sc(NC(=O)C(C)CNC(=O)c2cccc(-c3noc(C)n3)c2)nc1C]

Example 145.1.: To tert-butoxycarbonyl tert-butyl carbonate (2.07 g, 9.4931 mmol), 3-amino-2-methyl-propanoic acid hydrate (1.15 g, 9.4931 mmol) in dry THF (9.49 mL) was added TEA (1.46 mL, 10.443 mmol). The reaction was stirred at rt for the weekend. Volatiles were removed in vacuo and 3-(tert-butoxycarbonylamino)-2-methyl-propanoic acid (2.57 g, 133%, 12.645 mmol) taken straight on to next step without further purification. HPLC/MS m/z: 226.1 [M+Na]⁺, Rt (P): 0.97 min.

Example 145.2.: To 3-(tert-butoxycarbonylamino)-2-methyl-propanoic acid (100.00 mg, 0.4920 mmol) was added ethyl 2-amino-4-methyl-thiazole-5-carboxylate (91.63 mg, 0.4920 mmol), HOBt (132.97 mg, 0.9841 mmol) and dry DMF (2.42 mL). After stirring EDC.HCl (188.64 mg, 0.9841 mmol) was added. Stirred under nitrogen for 18 h at 60 0° C. Water (50 mL) added and extracted with EtOAc (2×40 mL). The organics were combined, washed with saturated NaHCO₃ (30 mL), brine (30 mL), dried over MgSO₄ and concentrated in vacuo to give a light brown oil. This was purified by silica gel column chromatography (eluent: 5-60% EtOAc in DCM) to afford ethyl 2-[[3-(tert-butoxycarbonylamino)-2-methyl-propanoyl]amino]-4-methyl-thiazole-5-carboxylate (57 mg, 31%, 0.1535 mmol). HPLC/MS m/z: 372.1 [M+H]⁺, Rt (P): 1.53 min.

Example 145.3.: To ethyl 2-[[3-(tert-butoxycarbonylamino)-2-methyl-propanoyl]amino]-4-methyl-thiazole-5-carboxylate (57 mg, 0.15 mmol) in dioxane (0.77 mL), was added 4M HCl in dioxane (0.77 mL, 3.0691 mmol) dropwise, while stirring at rt. The mixture was stirred 2.5 h at rt before concentrating in vacuo to give ethyl 2-[(3-amino-2-methyl-propanoyl)amino]-4-methyl-thiazole-5-carboxylate (42 mg) as a colorless powder. Taken on without further purification. HPLC/MS m/z: 272.1 [M+H]⁺, Rt (P): 1.04 min.

Example 145.4.: To ethyl 2-[(3-amino-2-methyl-propanoyl)amino]-4-methyl-thiazole-5-carboxylate (58.00 mg, 0.2138 mmol) was added 3-(5-methyl-1,2,4-oxadiazol-3-yl)benzoic acid (43.64 mg, 0.2138 mmol) DMF (1.07 mL), DIPEA (0.11 mL, 0.6413 mmol) and HATU (75.43 mg, 0.3206 mmol). The reaction mixture was stirred at rt overnight. Reaction mixture purified directly using reverse phase column chromatography (eluent: 30-100% MeOH in water (+0.1% formic acid modifier in both)) to afford ethyl 4-methyl-2-(2-methyl-3-(3-(5-methyl-1,2,4-oxadiazol-3-yl)benzamido)propanamido)thiazole-5-carboxylate (30 mg, 31%, 0.0656 mmol) as a tan-coloured powder. HPLC/MS m/z: 458.1 [M+H]⁺, Rt (S): 2.83 min. ¹H NMR (500 MHz, DMSO-d₆) δ 12.53 (s, 1H), 8.85 (t, J=5.8 Hz, 1H), 8.43 (t, J=1.7 Hz, 1H), 8.12 (dt, J=7.8, 1.4 Hz, 1H), 8.00 (dt, J=8.0, 1.4 Hz, 1H), 7.64 (t, J=7.8 Hz, 1H), 4.23 (q, J=7.1 Hz, 2H), 3.54-3.40 (m, 2H), 3.02 (sext, J=6.8 Hz, 1H), 2.68 (s, 3H), 2.52 (s, 3H), 1.27 (t, J=7.1 Hz, 3H), 1.17 (d, J=7.1 Hz, 3H).

### Example 146: 4-(3-((5-(ethoxycarbonyl)-4-methylthiazol-2-yl)amino)-1-(3-(5-methyl-1,2,4-oxadiazol-3-yl)benzamido)-3-oxopropyl)piperidin-1-ium formate

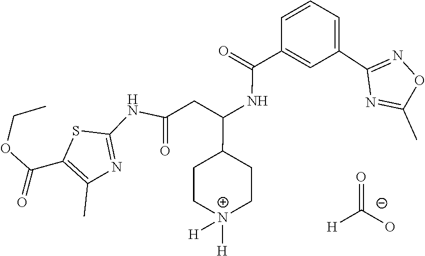 [SMILES: CCOC(=O)c1sc(NC(=O)CC(NC(=O)c2cccc(-c3noc(C)n3)c2)C2CC[NH2+]CC2)nc1C ; O=CO]

Example 146.1.: To tert-butyl 4-(1-amino-3-ethoxy-3-oxo-propyl)piperidine-1-carboxylate (125.05 mg, 0.4163 mmol) and 3-(5-methyl-1,2,4-oxadiazol-3-yl)benzoic acid (85.00 mg, 0.4163 mmol) in a 100 mL RBF under nitrogen atmosphere was added DMF (2.08 mL) followed by HATU (146.91 mg, 0.6244 mmol) and DIPEA (0.22 mL, 1.2489 mmol). The reaction mixture was stirred for 17 h before water (50 mL) added and extracted with EtOAc (2×30 mL). The organics were combined, washed with brine (30 mL), dried over MgSO₄, and concentrated in vacuo to give tert-butyl 4-[3-ethoxy-1-[[3-(5-methyl-1,2,4-oxadiazol-3-yl)benzoyl]amino]-3-oxo-propyl]piperidine-1-carboxylate (169 mg, 83%, 0.3473 mmol) as a clear gum. HPLC/MS m/z: 487.3 [M+H]⁺, Rt (R): 1.36 min.

Example 146.2.: To tert-butyl 4-[3-ethoxy-1-[[3-(5-methyl-1,2,4-oxadiazol-3-yl)benzoyl]amino]-3-oxo-propyl]piperidine-1-carboxylate (84.89 mg, 0.1745 mmol) in THF (0.87 mL) was added water (0.87 mL) followed by lithium hydroxide hydrate (29.32 mg, 0.6988 mmol). After stirring for 2.5 h water (20 mL) was added and THE was removed in vacuo. The crude 3-(1-tert-butoxycarbonyl-4-piperidyl)-3-[[3-(5-methyl-1,2,4-oxadiazol-3-yl)benzoyl]amino]propanoic acid was taken straight on to the next step. HPLC/MS m/z: 459.2 [M+H]⁺, Rt (R): 1.28 min.

Example 146.3.: To a solution of ethyl 2-amino-4-methyl-thiazole-5-carboxylate (35.74 mg, 0.1919 mmol), EDC.HCl (66.89 mg, 0.3490 mmol) in dry DMF (0.87 mL) was added 3-(1-tert-butoxycarbonyl-4-piperidyl)-3-[[3-(5-methyl-1,2,4-oxadiazol-3-yl)benzoyl]amino]propanoic acid (80.00 mg, 0.1745 mmol) and HOBt (47.15 mg, 0.3490 mmol). The mixture was stirred for 18.5 h at 60 0° C. Water (25 mL) added and extracted with EtOAc (2×25 mL). The organics were combined, washed with saturated NaHCO₃ (30 mL), brine (30 ml), dried over MgSO₄, and concentrated in vacuo to give ethyl 2-[[3-(1-tert-butoxycarbonyl-4-piperidyl)-3-[[3-(5-methyl-1,2,4-oxadiazol-3-yl)benzoyl]amino]propanoyl]amino]-4-methyl-thiazole-5-carboxylate as a yellow crude (76 mg) which was taken straight on to the next step. HPLC/MS m/z: 627.3 [M+H]⁺, Rt (P): 1.47 min.

Example 146.4.: Ethyl 2-[[3-(1-tert-butoxycarbonyl-4-piperidyl)-3-[[3-(5-methyl-1,2,4-oxadiazol-3-yl)benzoyl]amino]propanoyl]amino]-4-methyl-thiazole-5-carboxylate (76.00 mg, 0.1213 mmol) was dissolved in dioxane (1.00 mL) under nitrogen and 4M HCl in dioxane (1.00 mL, 4 mmol) was added. After 2 h volatiles were removed, the residue taken up in MeOH and added to a 2g SCX-II column which was washed with MeOH before eluting with 1:1 Mix of DCM and 7M NH₃ in MeOH. This was concentrated in vacuo to give a yellow crude which was purified by reverse phase column chromatography (eluent: 20-80% MeOH in water (+0.1% formic acid modifier in both)) to afford 4-(3-((5-(ethoxycarbonyl)-4-methylthiazol-2-yl)amino)-1-(3-(5-methyl-1,2,4-oxadiazol-3-yl)benzamido)-3-oxopropyl)piperidin-1-ium formate (8 mg, 12%, 0.0140 mmol) as clear glass. HPLC/MS m/z: 527.2 [M+H]⁺, Rt (Q): 2.45 min. ¹H NMR (500 MHz, Methanol-d₄) δ 8.50-8.39 (m, 2H), 8.17 (dt, J=7.8, 1.4 Hz, 1H), 7.93 (d, J=7.8 Hz, 1H), 7.60 (t, J=7.7 Hz, 1H), 4.55 (d, J=6.2 Hz, 1H), 4.26 (q, J=7.1 Hz, 2H), 3.46 (d, J=12.1 Hz, 2H), 3.08-2.91 (m, 3H), 2.85 (dd, J=15.1, 8.6 Hz, 1H), 2.65 (s, 4H), 2.53 (s, 3H), 2.07 (d, J=9.6 Hz, 3H), 1.63 (d, J=13.5 Hz, 2H), 1.32 (t, J=7.1 Hz, 3H).

### Example 147: 4-(3-((5-(ethoxycarbonyl)-4-methylthiazol-2-yl)amino)-1-(3-(5-methyl-1,2,4-oxadiazol-3-yl)benzamido)-3-oxopropyl)-1-methylpiperidin-1-ium formate

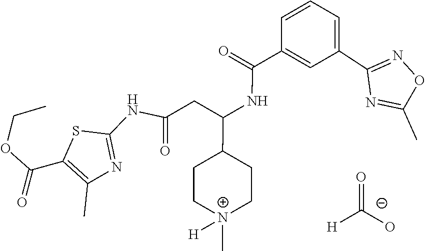 [SMILES: CCOC(=O)c1sc(NC(=O)CC(NC(=O)c2cccc(-c3noc(C)n3)c2)C2CC[NH+](C)CC2)nc1C ; O=CO]

Ethyl 4-methyl-2-[[3-[[3-(5-methyl-1,2,4-oxadiazol-3-yl)benzoyl]amino]-3-(4-piperidyl)propanoyl]amino]thiazole-5-carboxylate was synthesised in method analogous to the route to 0. To ethyl 4-methyl-2-[[3-[[3-(5-methyl-1,2,4-oxadiazol-3-yl)benzoyl]amino]-3-(4-piperidyl)propanoyl]amino]thiazole-5-carboxylate (20.00 mg, 0.0380 mmol) under nitrogen was added dry DCE (0.38 mL), AcOH (3 drops), and formaldehyde (34.5% wt in H₂O) (87.91 uL, 0.3798 mmol). The reaction mixture was stirred for 3 h before sodium triacetoxyborohydride (24.15 mg, 0.1139 mmol) was added and the mixture was stirred overnight at rt. Saturated NaHCO₃ solution (10 mL) added this was extracted with DCM (3×10 mL). The combined organics were washed with brine (10 mL), dried over MgSO₄ and concentrated in vacuo to give a clear crude (11 mg). Purified by reverse phase column chromatography (eluent: 20-80% MeOH in water (+0.1% formic acid modifier in both)) afforded ethyl 4-methyl-2-(3-(3-(5-methyl-1,2,4-oxadiazol-3-yl)benzamido)-3-(1-methylpiperidin-4-yl)propanamido)thiazole-5-carboxylate (2.5 mg, 12%, 0.0046 mmol) HPLC/MS m/z: 541.2 [M+H]⁺, Rt (S): 2.28 min. ¹H NMR (500 MHz, Methanol-d₄) δ 8.47-8.36 (m, 2H), 8.18 (dt, J=7.8, 1.4 Hz, 1H), 7.93 (dt, J=8.0, 1.3 Hz, 1H), 7.61 (t, J=7.8 Hz, 1H), 4.54 (q, J=7.7 Hz, 1H), 4.27 (q, J=7.2 Hz, 2H), 3.53 (d, J=12.3 Hz, 2H), 3.05-2.93 (m, 3H), 2.86-2.80 (m, 4H), 2.66 (s, 3H), 2.55 (s, 3H), 2.17-1.97 (m, 3H), 1.77-1.57 (m, 2H), 1.32 (t, J=7.1 Hz, 3H).

### Example 148: Ethyl-2-[[(3R)-4-amino-3-[[3-(5-methyl-1,2,4-oxadiazol-3-yl)benzoyl]amino]butanoyl]amino]-4-methyl-thiazole-5-carboxylate

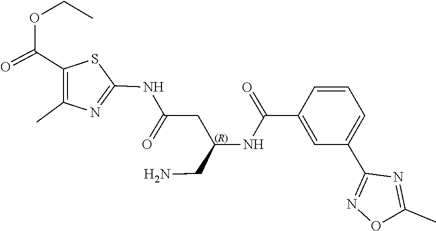

Example 148.1.: To a solution of ethyl 2-amino-4-methyl-thiazole-5-carboxylate (203.47 mg, 1.0926 mmol), Z-beta-Dab(Boc)-OH (350.00 mg, 0.9932 mmol) in dry DMF (4.97 mL) was added EDC.HCl (380.81 mg, 1.9865 mmol) and HOBt (268.41 mg, 1.9865 mmol). The mixture was stirred for 18.5 h at 60 0° C. Upon cooling EtOAc and water were added. The organic layer was retained, washed with brine, dried over MgSO₄ and concentrated in vacuo. Purified by silica gel column chromatography (eluent: 10-80% EtOAc in DCM) afforded ethyl 2-[[(3R)-3-(benzyloxycarbonylamino)-4-(tert-butoxycarbonylamino)butanoyl]amino]-4-methyl-thiazole-5-carboxylate (365 mg, 71%, 0.7011 mmol) as a colorless foam. HPLC/MS m/z: 521.2 [M+H]⁺, Rt (P): 1.60 min.

Example 148.2.: To ethyl 2-[[(3R)-3-(benzyloxycarbonylamino)-4-(tert-butoxycarbonylamino)butanoyl]amino]-4-methyl-thiazole-5-carboxylate (180.00 mg, 0.3458 mmol) in EtOH under nitrogen was carefully added 10% Palladium on carbon (12.00 mg, 0.1128 mmol). A hydrogen atmosphere was introduced and stirred at rt overnight. Another 12 mg of Pd/C was added and resubmitted to hydrogenation at 30° C. An extra 12 mg of Pd/C carefully added under nitrogen and resubmitted to hydrogen at rt for 66 h. Reaction mixture heated at 30° C. for 18 h. Reaction mixture was filtered through celite, washed with fresh EtOH. The filtrated was concentrated in vacuo to give a crude product. Purified by silica gel column chromatography (eluent: MeOH in DCM with 1% NH₃) to afford ethyl 2-[[(3R)-3-amino-4-(tert-butoxycarbonylamino)butanoyl]amino]-4-methyl-thiazole-5-carboxylate (35 mg, 26%, 0.0906 mmol) as a clear glass. HPLC/MS m/z: 387.2 [M+H]⁺, Rt (P): 1.24 min.

Example 148.3.: To ethyl 2-[[(3R)-3-amino-4-(tert-butoxycarbonylamino)-butanoyl]amino]-4-methyl-thiazole-5-carboxylate (35.00 mg, 0.0906 mmol), 3-(5-methyl-1,2,4-oxadiazol-3-yl)benzoic acid (18.49 mg, 0.0906 mmol) in DMF (0.45 mL) was added HATU (34.43 mg, 0.0906 mmol) and DIPEA (63.27 uL, 0.3623 mmol). After stirring at rt for 3 h 4M HCl in dioxane (0.45 mL, 1.8 mmol) was added and stirred at rt for 1.5 h. Stirred overnight but only showed partial deprotection. Mixture of 1 mL MeOH and 1 mL 4M HCl in dioxane added and stirred at rt for 4.5 h. The volatiles were removed in vacuo and the crude was purified by ion exchange SCX-II column which was washed with MeOH before eluting with 2M NH₃ in MeOH. Purified by reverse phase column chromatography (eluent: 20-90% MeOH in water (+0.1% formic acid modifier in both). Ion exchange SCX-II column used to afford ethyl 2-[[(3R)-4-amino-3-[[3-(5-methyl-1,2,4-oxadiazol-3-yl)benzoyl]amino]-butanoyl]amino]-4-methyl-thiazole-5-carboxylate (17 mg, 40%, 0.0360 mmol). HPLC/MS m/z: 473.2 [M+H]⁺, Rt (S): 2.15 min. ¹H NMR (500 MHz, Methanol-d₄) δ 8.45 (t, J=1.8 Hz, 1H), 8.13 (dt, J=7.8, 1.5 Hz, 1H), 8.01-7.89 (m, 1H), 7.56 (t, J=7.8 Hz, 1H), 4.59 (tt, J=7.4, 5.7 Hz, 1H), 4.25 (q, J=7.1 Hz, 2H), 3.07-2.76 (m, 4H), 2.64 (s, 3H), 2.52 (s, 3H), 1.31 (t, J=7.1 Hz, 3H).

### Example 149: Ethyl-4-methyl-2-[[(3S,4R)-4-[[3-(5-methyl-1,2,4-oxadiazol-3-yl)benzoyl]amino]pyrrolidine-3-carbonyl]amino]thiazole-5-carboxylate; formic acid

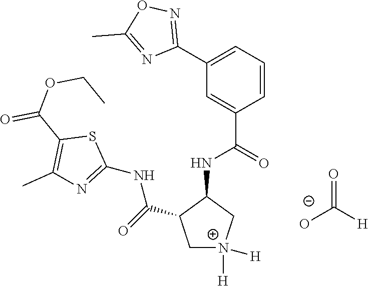

Example 149.1.: (3R,4S)-1-benzyl-4-(tert-butoxycarbonylamino)pyrrolidine-3-carboxylic acid (120.00 mg, 0.3746 mmol) suspended in methanol (3.75 mL, 0.3746 mmol) and five drops of H₂SO₄ added. Heated in microwave for 60 min at 75° C. Diluted with MeOH and passed through a Biotage 5 g ISOLUTE® NH₂ column to remove acid components. Concentrated in vacuo to leave a colorless solid (115 mg). Dissolved in MeOH and added to a 2g SCX-II column which was washed with MeOH before eluting with 2M NH₃ in MeOH. This was concentrated in vacuo to give methyl (3R,4S)-4-amino-1-benzyl-pyrrolidine-3-carboxylate (65 mg, 74%, 0.2774 mmol). HPLC/MS m/z: 235.14, [M+H]⁺, Rt (P): 0.09 min.

Example 149.2.: Procedure from 0 using methyl (3R,4S)-4-amino-1-benzyl-pyrrolidine-3-carboxylate. Purified by reverse phase column chromatography (eluent: 20-80% MeOH in water (+0.1% formic acid modifier in both) afforded methyl (3R,4S)-1-benzyl-4-[[3-(5-methyl-1,2,4-oxadiazol-3-yl)benzoyl]amino]-pyrrolidine-3-carboxylate (94 mg, 95%, 0.2236 mmol) as a colorless powder. HPLC/MS m/z: 421.19, [M+H]⁺, Rt (R): 0.92 min.

Example 149.3.: To methyl (3R,4S)-1-benzyl-4-[[3-(5-methyl-1,2,4-oxadiazol-3-yl)benzoyl]amino]pyrrolidine-3-carboxylate (40.00 mg, 0.0951 mmol) in THF (0.54 mL) was added water (0.54 mL) followed by lithium hydroxide hydrate (15.97 mg, 0.3805 mmol). After stirring for 4 h before water (20 mL) was added and the THE removed in vacuo. The solution was acidified with 1 M citric acid solution and extracted with EtOAc (3×15 mL). Basic components were recovered from organic and aqueous phases using ion exchange SCX-II column affording (3R,4S)-1-benzyl-4-[[3-(5-methyl-1,2,4-oxadiazol-3-yl)benzoyl]amino]pyrrolidine-3-carboxylic acid (26 mg, 67%, 0.0640 mmol) as a clear glass. HPLC/MS m/z: 407.2 [M+H]⁺, Rt (P): 1.16 min.

Example 149.4.: To (3R,4S)-1-benzyl-4-[[3-(5-methyl-1,2,4-oxadiazol-3-yl)benzoyl]amino]pyrrolidine-3-carboxylic acid (25.00 mg, 0.0615 mmol), ethyl 2-amino-4-methyl-thiazole-5-carboxylate (12.60 mg, 0.0677 mmol), HOBt (16.62 mg, 0.1230 mmol), and 3-(ethyliminomethyleneamino)-N,N-dimethyl-propan-1-amine hydrochloride (23.58 mg, 0.1230 mmol) under nitrogen was added DMF (0.31 mL) and the reaction mixture heated at 60° C. overnight. Volatiles were evaporated in vacuo and purified by reverse phase column chromatography (eluent: 30-100% MeOH in water (+0.1% formic acid modifier in both). Removal of formic acid by ion exchange SCX-II column afforded ethyl 2-[[(3S,4R)-1-benzyl-4-[[3-(5-methyl-1,2,4-oxadiazol-3-yl)benzoyl]amino]pyrrolidine-3-carbonyl]amino]-4-methyl-thiazole-5-carboxylate (12 mg, 34%, 0.0209 mmol) as a colorless powder. HPLC/MS m/z: 575.2 [M+H]⁺, Rt (S): 2.51 min.

Example 149.5.: To ethyl 2-[[(3S,4R)-1-benzyl-4-[[3-(5-methyl-1,2,4-oxadiazol-3-yl)benzoyl]amino]pyrrolidine-3-carbonyl]amino]-4-methyl-thiazole-5-carboxylate (11.00 mg, 0.0191 mmol) in a microwave vial under nitrogen was added dry DCE (0.25 mL) followed by 1-Chloroethyl chloroformate (6.20 uL, 0.0574 mmol) [3 drops]. The vial was heated at 90° C. for 1.5 h after which the solvent was evaporated and replaced with MeOH (0.75 mL). This was then heated for 1 h 10 min at 50° C. Only partial conversion, volatiles removed, and process repeated using 10 eq of 1-Chloroethyl chloroformate instead, 0.2 ml DCE and heated for 4 h as 90° C. The volatiles were removed, MeOH added and heated at 50° C. for 2 h. Purified by reverse phase column chromatography (eluent: 30-90% MeOH in water (+0.1% formic acid modifier in both)) afforded ethyl 4-methyl-2-[[(3S,4R)-4-[[3-(5-methyl-1,2,4-oxadiazol-3-yl)benzoyl]amino]pyrrolidine-3-carbonyl]amino]thiazole-5-carboxylate; formic acid (1.16 mg, 11% yield, 92% purity, 0.0020 mmol). HPLC/MS m/z: 485.2 [M+H]⁺, Rt (S): 2.29 min. ¹H NMR (500 MHz, Methanol-d₄) δ 8.58 (s, 1H), 8.46 (s, 1H), 8.26-8.21 (m, 1H), 8.06 (d, J=7.7 Hz, 1H), 7.66 (t, J=7.5 Hz, 1H), 4.77 (s, 1H), 4.32 (q, J=7.1 Hz, 2H), 3.76-3.44 (m, 5H), 2.68 (s, 3H), 2.59 (s, 3H), 1.37 (t, J=7.1 Hz, 3H).

### Example 150: Ethyl-(S)-2-(6-amino-3-(3-(5-methyl-1,2,4-oxadiazol-3-yl)benzamido)hexanamido)-4-methylthiazole-5-carboxylate

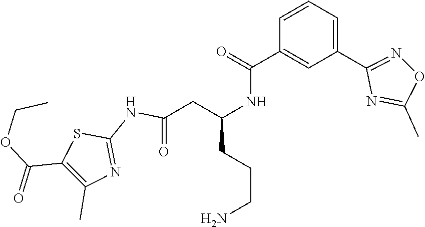 [SMILES: CCOC(=O)c1sc(NC(=O)C[C@H](CCCN)NC(=O)c2cccc(-c3noc(C)n3)c2)nc1C]

Example 150.1.: To 3-(5-methyl-1,2,4-oxadiazol-3-yl)benzoic acid (20.72 mg, 0.1015 mmol) and HATU (38.59 mg, 0.1015 mmol) under a nitrogen atmosphere was added dry DMF (0.51 mL) and DIPEA (0.04 mL, 0.2030 mmol). The reaction was stirred for 30 min at rt before the addition of (3S)-3-amino-6-(tert-butoxy-carbonylamino)hexanoic acid (25.00 mg, 0.1015 mmol). Reaction mixture stirred overnight. Partitioned between 5% NaHCO₃ (25 ml) and EtOAc (25 ml). Aqueous was retained, acidified with 0.1 M HCl and extracted with EtOAc (2×25 ml). The organics were combined and washed with brine to give crude (3S)-6-(tert-butoxycarbonylamino)-3-[[3-(5-methyl-1,2,4-oxadiazol-3-yl)benzoyl]amino]hexanoic acid (40 mg) which was used without purification. HPLC/MS m/z: 333.2 [M+H]⁺, Rt (R): 1.22 min.

Example 150.2.: To (3S)-6-(tert-butoxycarbonylamino)-3-[[3-(5-methyl-1,2,4-oxadiazol-3-yl)benzoyl]amino]hexanoic acid (40.00 mg), ethyl 2-amino-4-methyl-thiazole-5-carboxylate (18.95 mg, 0.1017 mmol) was added HOBt (25.00 mg, 0.1850 mmol) and EDC.HCl (35.46 mg, 0.1850 mmol). A nitrogen atmosphere was introduced followed by dry DMF (0.52 mL). The mixture was stirred for 18.5 h overnight at 60 0° C. The volatiles were removed in vacuo to leave a yellow gum which was partially purified by silica gel column chromatography (eluent: 2-10% MeOH in DCM) to afford ethyl 2-[[(3S)-6-(tert-butoxycarbonylamino)-3-[[3-(5-methyl-1,2,4-oxadiazol-3-yl)benzoyl]amino]hexanoyl]amino]-4-methyl-thiazole-5-carboxylate (40 mg). HPLC/MS m/z: 601.2 [M+H]⁺, Rt (R): 1.58 min.

Example 150.3.: Ethyl 2-[[(3S)-6-(tert-butoxycarbonylamino)-3-[[3-(5-methyl-1,2,4-oxadiazol-3-yl)benzoyl]amino]hexanoyl]amino]-4-methyl-thiazole-5-carboxylate (40.00 mg) was dissolved in dioxane (1.50 mL) under nitrogen and 4M HCl in dioxane (1.50 mL, 6 mmol) was added. After 2 h the volatiles were removed, and the crude product purified by reverse phase column chromatography (eluent: 30-90% MeOH in water (+0.1% formic acid modifier in both)). Ion exchange chromatography using SCX-II afforded ethyl (S)-2-(6-amino-3-(3-(5-methyl-1,2,4-oxadiazol-3-yl)benzamido)hexanamido)-4-methylthiazole-5-carboxylate (6.5 mg, 13% over 3 steps, 0.0130 mmol). HPLC/MS m/z: 501.2 [M+H]⁺, Rt (Q): 2.43 min. ¹H NMR (500 MHz, Methanol-d₄) δ 8.45 (t, J=1.8 Hz, 1H), 8.16 (dt, J=7.8, 1.4 Hz, 1H), 8.00-7.91 (m, 1H), 7.59 (t, J=7.8 Hz, 1H), 4.63-4.53 (m, 1H), 4.26 (q, J=7.1 Hz, 2H), 2.88-2.73 (m, 4H), 2.66 (s, 3H), 2.54 (s, 3H), 1.82-1.58 (m, 4H), 1.33 (t, J=7.1 Hz, 3H).

### Example 151: Popyl-(S)-2-(6-amino-3-(3-(5-methyl-1,2,4-oxadiazol-3-yl)benzamido)hexanamido)-4-methylthiazole-5-carboxylate

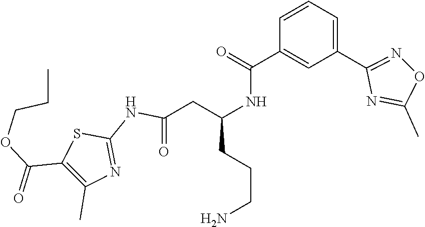 [SMILES: CCCOC(=O)c1sc(NC(=O)C[C@H](CCCN)NC(=O)c2cccc(-c3noc(C)n3)c2)nc1C]

Synthesised in an analogous manner to 0 afforded propyl (S)-2-(6-amino-3-(3-(5-methyl-1,2,4-oxadiazol-3-yl)benzamido)hexanamido)-4-methylthiazole-5-carboxylate (13.5 mg, 0.0262 mmol). HPLC/MS m/z: 515.2 [M+H]⁺, Rt (Q): 2.54 min. ¹H NMR (500 MHz, Methanol-d₄) δ 8.44 (t, J=1.7 Hz, 1H), 8.14 (dt, J=7.8, 1.4 Hz, 1H), 7.94 (ddd, J=7.8, 1.9, 1.2 Hz, 1H), 7.57 (t, J=7.8 Hz, 1H), 4.63-4.53 (m, 1H), 4.16 (t, J=6.6 Hz, 2H), 2.87-2.77 (m, 4H), 2.64 (s, 3H), 2.53 (s, 3H), 1.81-1.61 (m, 6H), 0.99 (t, J=7.4 Hz, 3H).

### Example 152: Propyl 4-methyl-2-[[(3S)-6-(methylamino)-3-[[3-(5-methyl-1,2,4-oxadiazol-3-yl)benzoyl]amino]hexanoyl]amino]thiazole-5-carboxylate

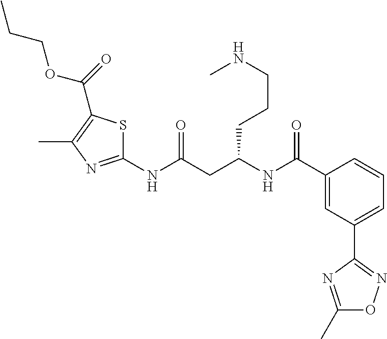 [SMILES: CCCOC(=O)c1sc(NC(=O)C[C@H](CCCNC)NC(=O)c2cccc(-c3noc(C)n3)c2)nc1C]

Example 152.1.: tert-Butyl N-(4-hydroxybutyl)-N-methyl-carbamate (1.00 g, 4.91 mmol, 1 eq) and Dess-Martin Periodinane (2.92 g, 6.88 mmol, 1.4 eq) were dissolved in DCM (15 mL). The reaction mixture was stirred at rt for 2 h then diluted with EtOAc and filtered through celite. The filtrate was washed with an aqueous solution of Na₂S20s, NaHCO₃, dried over MgSO₄ and concentrated under reduced pressure to yield tert-butyl methyl(4-oxobutyl)carbamate (990 mg) as a colourless oil. The product was used in the next step without further purification.

Example 152.2.: tert-Butyl methyl(4-oxobutyl)carbamate (990 mg, 4.92 mmol, 1 eq) and tert-butyl(triphenylphosphoranylidene)acetate (2.04 g, 5.41 mmol, 1.1 eq) were dissolved in dry PhMe (16.4 mL). The reaction mixture was heated to 120° C. and stirred for 16 h. The solvent was removed under reduced pressure and silica was added. The silica was added to a 50 g SNAP column and was eluted with a gradient of cyclohexane and EtOAc (15-40%) to give tert-butyl (E)-6-((tert-butoxycarbonyl)(methyl)amino)hex-2-enoate (1.02 g, 70% after 2 steps) as a yellow oil. ¹H NMR (500 MHz, Chloroform-d) δ 6.84 (dt, J=15.6, 6.8 Hz, 1H), 5.75 (dt, J=15.7, 1.6 Hz, 1H), 3.22 (s, 2H), 2.83 (s, 3H), 2.20-2.10 (m, 2H), 1.71-1.59 (m, 3H), 1.47 (s, 9H), 1.45 (s, 9H).

Example 152.3.: (S)-(−)-N-Benzyl-a-methylbenzylamine (1.26 mL, 6.02 mmol, 1.2 eq) was dissolved in dry THF (11.2 mL). The mixture was cooled to −78° C., n-BuLi (2.25 M in hexanes, 2.67 mL, 6.02 mmol, 1.2 eq) was added and the reaction mixture was stirred at −78° C. for 15 min. Compound tert-butyl (E)-6-((tert-butoxycarbonyl)(methyl)amino)hex-2-enoate (1.50 g, 5.00 mmol, 1 eq), in dry THE (1.5 mL) was added to the solution. The reaction mixture was stirred at −78° C. for 1 h before being quenched with water. The product was extracted with EtOAc, dried over MgSO₄ and concentrated under reduced pressure to afford tert-butyl (S)-3-(benzyl((S)-1-phenylethyl)amino)-6-((tert-butoxycarbonyl)(methyl)amino)hexanoate (117 mg, 69%) as a yellow oil. HPLC/MS m/z: 511.4, [M+H]⁺, Rt (R): 1.73 min.

Example 152.4.: MeOH (18.7 mL) was added to Pd(OH)₂ (351 mg, 0.50 mmol, 0.1 eq). Ammomium formate (1.57 g, 25.00 mmol, 5 eq) was slowly added per batches over 5 min (caution gas evolved) and the mixture was stirred for 10 min at rt. tert-Butyl (S)-3-(benzyl((S)-1-phenylethyl)amino)-6-((tert-butoxycarbonyl)(methyl)-amino)hexanoate (2.55 g, 5.00 mmol, 1 eq), in MeOH (2 mL), was slowly added to the mixture, stirred for 15 min then formic acid (1.27 mL) was added. The reaction mixture was stirred 60° C. for 16 h. Ammonium formate (1.57 g, 25.00 mmol, 5 eq), formic acid (0.6 mL) and Pd(OH)₂ (70 mg, 0.25 mmol, 0.05 eq) were added, stirred at 60° C. for 24 h. The reaction mixture was cooled down to rt then filtered through celite, washed with MeOH and concentrated under reduced pressure. The crude was added to a 50 g SNAP column and was eluted with a gradient of DCM and MeOH (0-10%), to yield tert-butyl (S)-3-amino-6-((tert-butoxycarbonyl)(methyl)amino)hexanoate (800 mg, 51%) as a colourless oil. HPLC/MS m/z: 317.2, [M+H]⁺, Rt (T): 1.14 min.

Example 152.5.: 3-(5-methyl-1,2,4-oxadiazol-3-yl)benzoic acid (484 mg, 2.37 mmol, 1.5 eq), tert-butyl (S)-3-amino-6-((tert-butoxycarbonyl)(methyl)amino)hexanoate (500 mg, 1.58 mmol, 1.0 eq) were dissolved in dry DMF (8.0 mL). Triethylamine (0.66 mL, 4.74 mmol, 3 eq) and T₃P (50% in DMF, 0.70 mL, 1.5 eq) were added. The reaction mixture was stirred at rt for 2 h then diluted with EtOAc and water. The organic layer was washed with NaHCO₃, dried over MgSO₄ and concentrated in vacuo. The crude was added to a 50 g SNAP column and was eluted with a gradient of cyclohexane and EtOAc (20-50%) to yield tert-butyl (S)-6-((tert-butoxycarbonyl)(methyl)amino)-3-(3-(5-methyl-1,2,4-oxadiazol-3-yl)benzamido)hexanoate (630 mg, 79%) as a colourless oil. HPLC/MS m/z: 525.3, [M+H]⁺, Rt (T): 1.50 min.

Example 152.6.: tert-butyl (S)-6-((tert-butoxycarbonyl)(methyl)amino)-3-(3-(5-methyl-1,2,4-oxadiazol-3-yl)benzamido)hexanoate (430 mg, 0.85 mmol, 1 eq) and potassium hydroxide (960 mg, 17.1 mmol, 20 eq) were dissolved in THF (4.30 mL). Few drops of water and MeOH (enough to dissolve all the KOH) were added and the reaction mixture was stirred at 50° C. for 5 h. The pH was adjusted to pH=3 with a 1 M solution of HCl. The product was extrated with EtOAc, dried over MgSO₄ and concentrated in vacuo. The crude was dissolved in DMSO (1.5 mL+0.3 mL) and purified via column chromatography (KP-C18, 30 g, 30-100% MeOH/H₂O+0.1% formic acid) to give (S)-6-((tert-butoxycarbonyl)(methyl)amino)-3-(3-(5-methyl-1,2,4-oxadiazol-3-yl)benzamido)hexanoic acid (170 mg, 44%) as a colorless powder. HPLC/MS m/z: 447.2, [M+H]⁺, Rt (T): 1.32 min.

Example 152.7.: A mixture of (S)-6-((tert-butoxycarbonyl)(methyl)amino)-3-(3-(5-methyl-1,2,4-oxadiazol-3-yl)benzamido)hexanoic acid (145.00 mg, 0.3247 mmol), EDC.HCl (124.51 mg, 0.6495 mmol), HOBt (87.76 mg, 0.6495 mmol), propyl 2-amino-4-methyl-thiazole-5-carboxylate (97.55 mg, 0.4871 mmol) and DMF (1.62 mL) under nitrogen atmosphere was stirred at 45° C. for 17.5 h. The mixture was cooled down to rt and partitioned between EtOAc (75 mL) and water (50 mL). The aqueous layer was further extracted with EtOAc (50 mL), the combined organic layers were washed with water (50 mL), 1 M aqueous solution of HCl (50 mL), aqueous saturated solution of NaHCO₃ (75 mL) and brine (75 mL), dried with MgSO₄, filtered, and evaporated to dryness. Purified by reverse phase column chromatography (eluent: 40-100% MeOH/H₂O+0.1% formic acid) to give propyl 2-[[(3S)-6-[tert-butoxycarbonyl(methyl)amino]-3-[[3-(5-methyl-1,2,4-oxadiazol-3-yl)benzoyl]amino]hexanoyl]amino]-4-methyl-thiazole-5-carboxylate (102.4 mg, 50%, 0.1629 mmol) as a colorless powder. HPLC/MS m/z: 629.3, [M+H]⁺, Rt (P): 1.64 min.

Example 152.8.: Propyl 2-[[(3S)-6-[tert-butoxycarbonyl(methyl)amino]-3-[[3-(5-methyl-1,2,4-oxadiazol-3-yl)benzoyl]amino]hexanoyl]amino]-4-methyl-thiazole-5-carboxylate (102.00 mg, 0.1622 mmol) was dissolved in dry DCM (1.62 mL). Trifluoroacetic acid (248.29 uL, 3.2446 mmol) was added and the reaction mixture was stirred at rt for 45 min. The volatiles were removed under vacuo and the obtained clear oil (173 mg) was dissolved in MeOH and purified by ion exchange SCX-II column chromatography, washing with MeOH before eluting with 2M NH₃ in MeOH. Evaporated to dryness in vacuo to give propyl 4-methyl-2-[[(3S)-6-(methylamino)-3-[[3-(5-methyl-1,2,4-oxadiazol-3-yl)benzoyl]amino]hexanoyl]-amino]thiazole-5-carboxylate (84 mg, 98%, 0.1589 mmol) as a colorless powder. HPLC/MS m/z: 529.2, [M+H]⁺, Rt (P): 1.35 min. ¹H NMR (600 MHz, Methanol-d₄) δ 8.46 (s, 1H), 8.17 (dt, J=7.8, 1.4 Hz, 1H), 7.95 (dt, J=7.9, 1.5 Hz, 1H), 7.60 (t, J=7.8 Hz, 1H), 4.60-4.53 (m, 1H), 4.19 (t, J=6.5 Hz, 2H), 2.84-2.76 (m, 2H), 2.73-2.64 (m, 5H), 2.55 (s, 3H), 2.42 (s, 3H), 1.80-1.60 (m, 6H), 1.00 (t, J=7.5 Hz, 3H).

The following examples were prepared in an analogous manner:

### Example 153: Propyl-(R)-4-methyl-2-(3-(3-(5-methyl-1,2,4-oxadiazol-3-yl)benzamido)-6-(methylamino)hexanamido)thiazole-5-carboxylate

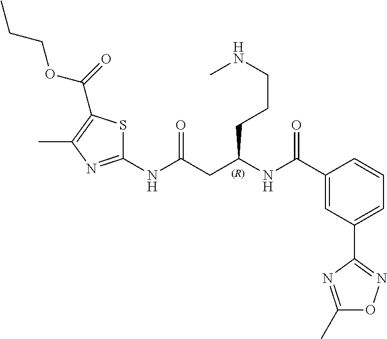

Using (R)-(−)-N-Benzyl-a-methylbenzylamine, propyl (R)-4-methyl-2-(3-(3-(5-methyl-1,2,4-oxadiazol-3-yl)benzamido)-6-(methylamino)hexanamido)thiazole-5-carboxylate (45 mg, 67%) was obtained as a colorless powder. HPLC/MS m/z: 529.2, [M+H]⁺, Rt (P): 1.34 min. ¹H NMR (600 MHz, Methanol-d₄) δ 8.45 (t, J=1.7 Hz, 1H), 8.16 (dt, J=7.8, 1.4 Hz, 1H), 7.97-7.93 (m, 1H), 7.59 (t, J=7.8 Hz, 1H), 4.63-4.53 (m, 1H), 4.18 (t, J=6.5 Hz, 2H), 2.85-2.79 (m, 2H), 2.77-2.70 (m, 2H), 2.65 (s, 3H), 2.55 (s, 3H), 2.45 (s, 3H), 1.80-1.63 (m, 6H), 1.00 (t, J=7.4 Hz, 3H).

### Example 154: Tert-butyl 4-methyl-2-[[(3S)-6-(methylamino)-3-[[3-(5-methyl-1,2,4-oxadiazol-3-yl)benzoyl]amino]hexanoyl]amino]thiazole-5-carboxylate

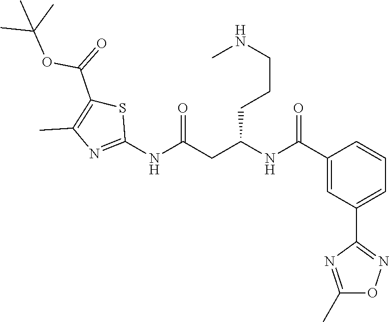 [SMILES: CNCCC[C@@H](CC(=O)Nc1nc(C)c(C(=O)OC(C)(C)C)s1)NC(=O)c1cccc(-c2noc(C)n2)c1]

15 mg (88%), colorless amorphous powder. HPLC/MS m/z: 543.24 [M+H]⁺, Rt (T): 1.26 min. ¹H NMR (500 MHz, Methanol-d₄) δ 8.50-8.45 (m, 1H), 8.24-8.17 (m, 1H), 7.97 (ddd, J=7.7, 1.9, 1.2 Hz, 1H), 7.63 (td, J=7.8, 0.6 Hz, 1H), 4.58 (quint, J=6.7 Hz, 1H), 2.82 (dd, J=6.7, 2.3 Hz, 2H), 2.75-2.69 (m, 2H), 2.68 (s, 3H), 2.55 (s, 3H), 2.44 (s, 3H), 1.89-1.64 (m, 4H), 1.56 (s, 9H).

### Example 155: Cyclopentyl (S)-4-methyl-2-(3-(3-(5-methyl-1,2,4-oxadiazol-3-yl)benzamido)-6-(methylamino)hexanamido)thiazole-5-carboxylate

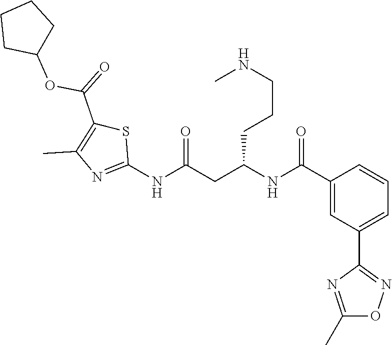 [SMILES: CNCCC[C@@H](CC(=O)Nc1nc(C)c(C(=O)OC2CCCC2)s1)NC(=O)c1cccc(-c2noc(C)n2)c1]

35 mg (78%), colorless powder. HPLC/MS m/z: 555.2, [M+H]⁺, Rt (T): 1.31 min. ¹H NMR (600 MHz, DMSO-d₆) δ 8.68 (s, 1H), 8.46-8.41 (m, 1H), 8.12 (dt, J=7.7, 1.4 Hz, 1H), 8.02 (dt, J=7.8, 1.4 Hz, 1H), 7.65 (t, J=7.7 Hz, 1H), 5.25-5.18 (m, 1H), 4.47-4.38 (m, 1H), 2.75-2.65 (m, 5H), 2.54 (t, J=7.4 Hz, 2H), 2.50 (s, 3H), 2.29 (s, 3H), 1.90-1.81 (m, 2H), 1.72-1.64 (m, 4H), 1.64-1.55 (m, 4H), 1.54-1.41 (m, 2H) (2×NH not observed)

### Example 156: Isopropyl (S)-4-methyl-2-(3-(3-(5-methyl-1,2,4-oxadiazol-3-yl)benzamido)-6-(methylamino)hexanamido)thiazole-5-carboxylate

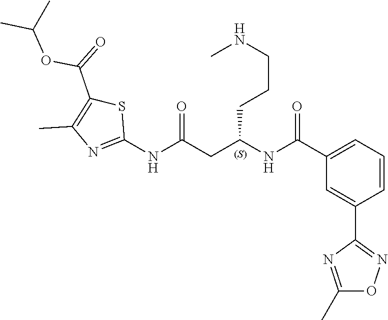

28 mg (90%), colorless powder. HPLC/MS m/z: 529.2, [M+H]⁺, Rt (T): 1.22 min. ¹H NMR (600 MHz, DMSO-d₆) δ 8.69 (s, 1H), 8.47-8.42 (m, 1H), 8.13 (dt, J=7.7, 1.4 Hz, 1H), 8.03 (dt, J=7.8, 1.5 Hz, 1H), 7.66 (t, J=7.8 Hz, 1H), 5.03 (hept, J=6.2 Hz, 1H), 4.48-4.40 (m, 1H), 2.77-2.64 (m, 5H), 2.55-2.51 (m, 5H), 2.30 (s, 3H), 1.65-1.57 (m, 2H), 1.54-1.41 (m, 2H), 1.26 (d, J=6.2 Hz, 6H). (2×NH not observed)

### Example 157: (S)-N-(1-((5-(tert-butyl)-4-methylthiazol-2-yl)amino)-6-(methylamino)-1-oxohexan-3-yl)-3-chloro-5-(5-methyl-1,2,4-oxadiazol-3-yl)benzamide

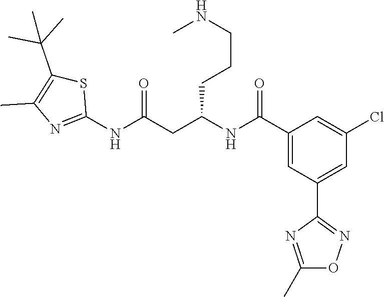 [SMILES: CNCCC[C@@H](CC(=O)Nc1nc(C)c(C(C)(C)C)s1)NC(=O)c1cc(Cl)cc(-c2noc(C)n2)c1]

1.24 mg (3%), colorless powder. HPLC/MS m/z: 533.2, [M+H]⁺, Rt (T): 1.36 min. ¹H NMR (600 MHz, Methanol-d₄) δ 8.38 (t, J=1.5 Hz, 1H), 8.15 (t, J=1.8 Hz, 1H), 7.95 (t, J=1.8 Hz, 1H), 4.55 (sext, J=6.6, 6.2 Hz, 1H), 2.82-2.62 (m, 7H), 2.44 (s, 3H), 2.33 (s, 3H), 1.78-1.60 (m, 4H), 1.39 (s, 9H).

### Example 158: Propyl (S)-2-(6-(dimethylamino)-3-(3-(5-methyl-1,2,4-oxadiazol-3-yl)benzamido)hexanamido)-4-methylthiazole-5-carboxylate

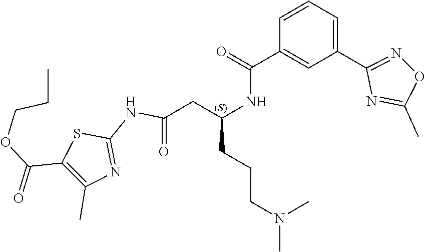

To propyl (S)-2-(6-amino-3-(3-(5-methyl-1,2,4-oxadiazol-3-yl)benzamido)hexan-amido)-4-methylthiazole-5-carboxylate [0](28.00 mg, 0.0544 mmol) under nitrogen was added dry DCE (0.52 mL), formaldehyde (34.5% wt in H₂O) (126.00 uL, 4.5734 mmol) and AcOH (2 drops). The reaction mixture was stirred for 15 min before sodium triacetoxyborohydride (34.60 mg, 0.1632 mmol) was added and stirred at rt for 22 h. Saturated NaHCO₃ solution (10 mL) was added this was extracted with DCM (3×10 mL) using a phase separator to remove the organics. The organics were combined and concentrated in vacuo to give a clear crude product (32 mg) which was purified by amino capped silica gel column chromatography (Biotage KP-NH silica, eluent: 2-10% EtOH in DCM) to afford propyl (S)-2-(6-(dimethylamino)-3-(3-(5-methyl-1,2,4-oxadiazol-3-yl)benzamido)hexanamido)-4-methylthiazole-5-carboxylate (10 mg, 34%, 0.0184 mmol) as a clear glass). HPLC/MS m/z: 543.2 [M+H]⁺, Rt (S): 2.45 min. ¹H NMR (500 MHz, Methanol-d₄) δ 8.45 (t, J=1.7 Hz, 1H), 8.17 (dt, J=7.7, 1.4 Hz, 1H), 7.94 (ddd, J=7.8, 1.9, 1.1 Hz, 1H), 7.60 (t, J=7.8 Hz, 1H), 4.58 (quint, J=6.8 Hz, 1H), 4.18 (t, J=6.5 Hz, 2H), 2.83 (dd, J=6.7, 2.5 Hz, 2H), 2.65 (s, 3H), 2.55 (s, 3H), 2.54-2.46 (m, 2H), 2.34 (s, 6H), 1.78-1.62 (m, 6H), 1.00 (t, J=7.4 Hz, 3H).

### Example 159: Propyl (S)-4-methyl-2-(3-(3-(5-methyl-1,2,4-oxadiazol-3-yl)benzamido)-6-morpholinohexanamido)thiazole-5-carboxylate

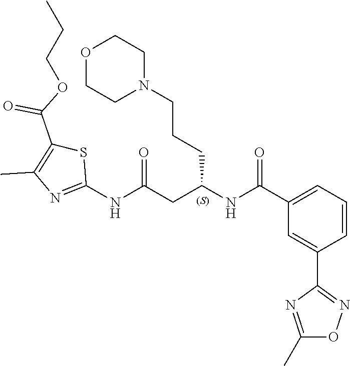

Example 159.1.: Synthesised in an analogous manner to 0 from methyl (3S)-3-amino-6-(tert-butoxycarbonylamino)hexanoate [0] to afford methyl (3S)-6-(tert-butoxycarbonylamino)-3-[[3-(5-methyl-1,2,4-oxadiazol-3-yl)benzoyl]amino]hexanoate (281 mg, 76%, 0.6293 mmol) as a colorless solid. HPLC/MS m/z: 347.19, [M-Boc+H]⁺, Rt (P): 1.44 min.

Example 159.2.: Methyl (3S)-6-(tert-butoxycarbonylamino)-3-[[3-(5-methyl-1,2,4-oxadiazol-3-yl)benzoyl]amino]hexanoate (278.00 mg, 0.6226 mmol) under nitrogen atmosphere was dissolved in dry MeOH (2.00 mL) and 4M HCl in dioxane (2.00 mL, 8 mmol) was added. Stirred at rt for 2.5 h before concentrated in vacuo. Purification by ion exchange SCX-II column gave methyl (3S)-6-amino-3-[[3-(5-methyl-1,2,4-oxadiazol-3-yl)benzoyl]amino]hexanoate (208 mg, 96%, 0.6005 mmol) as a clear gum. HPLC/MS m/z: 347.2 [M+H]⁺, Rt (P): 0.95 min.

Example 159.3.: To methyl (3S)-6-amino-3-[[3-(5-methyl-1,2,4-oxadiazol-3-yl)benzoyl]amino]hexanoate (44.00 mg, 0.1270 mmol) under a nitrogen atmosphere was added dry MeCN (0.85 mL), triethylamine (35.41 uL, 0.2541 mmol) and 1-bromo-2-(2-bromoethoxy)ethane (23.95 uL, 0.1905 mmol). The mixture was heated at 80° C. overnight. MeCN was removed in vacuo and the crude product purified by reverse phase column chromatography (eluent: 20-80% MeOH in water (+0.1% formic acid modifier in both)) to afford methyl (3S)-3-[[3-(5-methyl-1,2,4-oxadiazol-3-yl)benzoyl]amino]-6-morpholino-hexanoate formate (44 mg, 75%, 0.0951 mmol). HPLC/MS m/z: 417.2 [M+H]⁺, Rt (P): 0.99 min.

Example 159.4.: Synthesised in an analogous manner to ethyl 2-[[(3S,4R)-1-benzyl-4-[[3-(5-methyl-1,2,4-oxadiazol-3-yl)benzoyl]amino]pyrrolidine-3-carbonyl]amino]-4-methyl-thiazole-5-carboxylate. Purification by reverse phase column chromatography (eluent: 30-100% MeOH in water (+0.1% formic acid modifier in both)) followed by silica gel column chromatography (eluent: 2-10% MeOH in DCM+1% NH₃) afforded propyl (S)-4-methyl-2-(3-(3-(5-methyl-1,2,4-oxadiazol-3-yl)benzamido)-6-morpholinohexanamido)thiazole-5-carboxylate (1.56 mg, 4%, 0.0027 mmol). HPLC/MS m/z: 585.2 [M+H]⁺, Rt (Q): 2.57 min. ¹H NMR (600 MHz, Methanol-d₄) δ 8.45 (s, 1H), 8.18 (dt, J=7.8, 1.4 Hz, 1H), 7.94 (dt, J=7.8, 1.4 Hz, 1H), 7.61 (t, J=7.8 Hz, 1H), 4.60-4.54 (m, 1H), 4.20 (t, J=6.5 Hz, 2H), 3.66 (t, J=4.7 Hz, 4H), 2.81 (dd, J=6.8, 2.6 Hz, 2H), 2.66 (s, 3H), 2.57 (s, 3H), 2.52-2.36 (m, 6H), 1.82-1.70 (m, 4H), 1.70-1.60 (m, 2H), 1.00 (t, J=7.4 Hz, 3H).

### Example 160: Propyl (S)-2-(2-amino-3-(3-(5-methyl-1,2,4-oxadiazol-3-yl)benzamido)propanamido)-4-methylthiazole-5-carboxylate

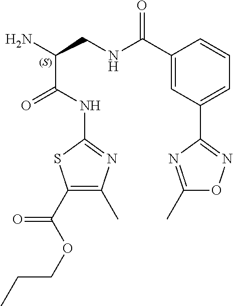

Example 160.1.: Synthesised from (S)-tert-Butyl-3-amino-2-((tert-butoxycarbonyl)amino)propanoate in an analogous manner to 0 to afford tert-butyl (2S)-2-(tert-butoxycarbonylamino)-3-[[3-(5-methyl-1,2,4-oxadiazol-3-yl)benzoyl]amino]propanoate (133 mg, 78%, 0.2979 mmol) as a clear gum. HPLC/MS m/z: 347.2 [M-Boc+H]⁺, Rt (P): 1.54 min.

Example 160.2.: tert-Butyl (2S)-2-(tert-butoxycarbonylamino)-3-[[3-(5-methyl-1,2,4-oxadiazol-3-yl)benzoyl]amino]propanoate was dissolved in THF (2.91 mL) and 2M

KOH (1.00 mL, 2.009 mmol) was added. The reaction mixture was stirred at 50° C. for 3 h. Concentrated in vacuo and purified by reverse phase column chromatography (eluent: 30-100% MeOH in water (+0.1% formic acid modifier in both)) to afford (2S)-2-(tert-butoxycarbonylamino)-3-[[3-(5-methyl-1,2,4-oxadiazol-3-yl)benzoyl]amino]propanoic acid (63 mg, 55%, 0.1614 mmol). HPLC/MS m/z: 291.11, [M-Boc+H]⁺, Rt (P): 1.36 min.

Example 160.3.: Synthesised in an analogous manner to 0. Purified by reverse phase column chromatography (eluent: 30-100% MeOH in water (+0.1% formic acid modifier in both) to afford propyl (S)-2-(2-((tert-butoxycarbonyl)amino)-3-(3-(5-methyl-1,2,4-oxadiazol-3-yl)benzamido)propanamido)-4-methylthiazole-5-carboxylate formate (26 mg, 59%, 0.0454 mmol). HPLC/MS m/z: 573.2 [M+H]⁺, Rt (Q):3.11 min.

Examplel160.4.: Propyl (S)-2-(2-((tert-butoxycarbonyl)amino)-3-(3-(5-methyl-1,2,4-oxadiazol-3-yl)benzamido)propanamido)-4-methylthiazole-5-carboxylate formate (20.00 mg, 0.0349 mmol) was dissolved in n-propanol (0.50 mL) and 4M HCl in dioxane (0.50 mL, 2 mmol) was added. The reaction was stirred for 2 h at rt before the volatiles were removed in vacuo. Purified by reverse phase column chromatography (eluent: 20-100% MeOH in water (+0.1% formic acid modifier in both)) and ion exchange SCX-II chromatography to afford propyl 2-[[(2S)-2-amino-3-[[3-(5-methyl-1,2,4-oxadiazol-3-yl)benzoyl]amino]propanoyl]amino]-4-methyl-thiazole-5-carboxylate (10 mg, 61%, 0.0212 mmol). HPLC/MS m/z: 473.2 [M+H]⁺, Rt (Q): 2.57 min. ¹H NMR (600 MHz, DMSO-d₆) δ 8.77 (t, J=5.8 Hz, 1H), 8.43 (t, J=1.8 Hz, 1H), 8.12 (dt, J=7.7, 1.4 Hz, 1H), 8.01 (dt, J=7.8, 1.4 Hz, 1H), 7.65 (t, J=7.8 Hz, 1H), 5.98 (s, 2H), 4.14 (t, J=6.5 Hz, 2H), 3.76 (t, J=6.4 Hz, 1H), 3.57 (t, J=6.1 Hz, 2H), 2.68 (s, 3H), 2.52 (s, 3H), 1.66 (sext, J=7.4 Hz, 2H), 0.93 (t, J=7.4 Hz, 3H).

### Example 161: (S)-N-(2-amino-3-((5-(tert-butyl)-4-ethylthiazol-2-yl)amino)-3-oxopropyl)-3-(5-methyl-1,2,4-oxadiazol-3-yl)benzamide

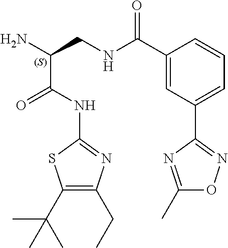

Synthesised in an analogous manner to 0 using 5-tert-butyl-4-ethyl-1,3-thiazol-2-amine. (S)-N-(2-amino-3-((5-(tert-butyl)-4-ethylthiazol-2-yl)amino)-3-oxopropyl)-3-(5-methyl-1,2,4-oxadiazol-3-yl)benzamide (20 mg, 0.0438 mmol). HPLC/MS m/z: 439.2 [M+H]⁺, Rt (U): 2.62 min. ¹H NMR (600 MHz, DMSO-d₆) δ 8.71 (t, J=5.8 Hz, 1H), 8.45 (t, J=1.8 Hz, 1H), 8.12 (dt, J=7.7, 1.4 Hz, 1H), 8.02 (dt, J=7.8, 1.5 Hz, 1H), 7.65 (t, J=7.8 Hz, 1H), 5.36 (s, 3H), 3.66 (t, J=6.4 Hz, 1H), 3.54 (dt, J=13.2, 5.9 Hz, 1H), 3.47 (dt, J=13.0, 6.3 Hz, 1H), 2.68 (s, 3H), 2.65 (q, J=7.5 Hz, 2H), 1.36 (s, 9H), 1.15 (t, J=7.5 Hz, 3H).

### Example 162: Propyl (S)-2-(5-(dimethylamino)-3-(3-(5-methyl-1,2,4-oxadiazol-3-yl)benzamido)pentanamido)-4-methylthiazole-5-carboxylate

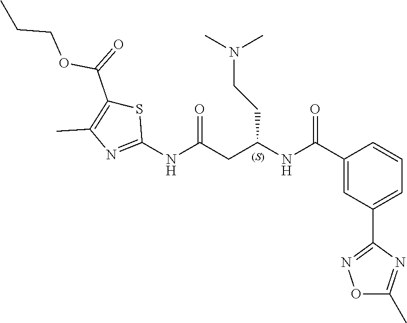

Example 162.1: (3S)-3-(benzyloxycarbonylamino)-5-tert-butoxy-5-oxo-pentanoic acid (348.00 mg, 1.0315 mmol) under nitrogen was dissolved in THF (1.21 mL) and cooled in an ice-salt bath. 1M Borane in THF (6.19 mL, 6.1891 mmol) was added dropwise over 20 min and the reaction mixture stirred at −5° C. for 2.5 h. MeOH (3 mL) carefully added and the volatiles were removed in vacuo. Partitioned between EtOAc (25 mL) and saturated NaHCO₃ solution (40 mL). The organic was retained and the aqueous extracted with further EtOAc (25 mL). The organics were combined, washed with brine (30 mL), dried over MgSO₄ and concentrated in vacuo. Purified by silica gel column chromatography (eluent: 5-59% EtOAc in DCM) to afford tert-butyl (3S)-3-(benzyloxycarbonylamino)-5-hydroxy-pentanoate (158 mg, 47%, 0.4886 mmol). HPLC/MS m/z: 346.2 [M+Na]⁺, Rt (P): 1.44 min.

Example 162.2.: To tert-butyl (3S)-3-(benzyloxycarbonylamino)-5-hydroxy-pentanoate (155.00 mg, 0.4793 mmol) in DCM (1.5 ml) under nitrogen at rt was added Dess Martin reagent (0.3 M in DCM) (2.08 mL, 0.6231 mmol) dropwise. After stirring at rt for 2.5 h the reaction mixture was diluted with DCM solution (20 mL) and partitioned against 10% aqueous sodium thiosulfate (40 mL). The aqueous was extracted with fresh DCM (20 mL), the organics combined, washed with saturated NaHCO₃ (20 mL), dried over Na₂SO₄ and concentrated in vacuo to give tert-butyl (3S)-3-(benzyloxycarbonylamino)-5-oxo-pentanoate as a clear gum (208 mg) which was used immediately in the next reductive amination step.

Example 162.3.: Sodium triacetoxyborohydride (152.34 mg, 0.7188 mmol) was added to a solution of tert-butyl (3S)-3-(benzyloxycarbonylamino)-5-oxo-pentanoate (154.00 mg, 0.4792 mmol) and dimethylamine (2M in THF) (0.36 mL, 0.7188 mmol) in THF (4.79 mL) under nitrogen. Acetic acid (5 drops) was added, and the white suspension was stirred overnight at rt. MeOH/water added and volatiles were removed in vacuo. Purified by ion exchange SCX-II column chromatography washing with MeOH before eluting basic components with 2M NH₃ in MeOH. Concentrated in vacuo to give tert-butyl (3S)-3-(benzyloxycarbonylamino)-5-(dimethylamino)pentanoate (133 mg, 79%, 0.3795 mmol) as a clear gum. HPLC/MS m/z: 351.2 [M+H]⁺, Rt (P): 1.14 min.

Example 162.4.: tert-Butyl (3S)-3-(benzyloxycarbonylamino)-5-(dimethylamino)pentanoate (70.00 mg, 0.1997 mmol) was dissolved in EtOH (1.00 mL) and, under nitrogen, 10% Pd on Carbon (21.26 mg, 0.0200 mmol) was carefully added. A hydrogen atmosphere was introduced. Stirred overnight at rt. Filtered through celite and washed throughly with fresh EtOH. Concentrated in vacuo to give tert-butyl (3S)-3-amino-5-(dimethylamino)pentanoate (27 mg, 62%, 0.1248 mmol) as a yellow oil. HPLC/MS m/z: 217.2 [M+H]⁺, Rt (P): 0.09 min.

Example 162.5.: To tert-butyl (3S)-3-amino-5-(dimethylamino)pentanoate (25.00 mg, 0.1156 mmol), 3-(5-methyl-1,2,4-oxadiazol-3-yl)benzoic acid (25.96 mg, 0.1271 mmol), and HATU (48.34 mg, 0.1271 mmol) under nitrogen was added dry DMF (0.77 mL) and DIPEA (0.04 mL, 0.2311 mmol). The reaction was stirred overnight at rt. LCMS showed no further conversion. Purified by reverse phase column chromatography (eluent: 20-80% MeOH in water (+0.1% formic acid modifier in both)) to afford tert-butyl (3S)-5-(dimethylamino)-3-[[3-(5-methyl-1,2,4-oxadiazol-3-yl)benzoyl]amino]pentanoate; formic acid (18 mg, 35%, 0.0401 mmol). HPLC/MS m/z: 403.2 [M+H]⁺, Rt (R): 1.20 min.

Example 162.6.: tert-Butyl (3S)-5-(dimethylamino)-3-[[3-(5-methyl-1,2,4-oxadiazol-3-yl)benzoyl]amino]pentanoate; formic acid (17.00 mg, 0.0422 mmol) was stirred at rt in trifluoroacetic acid (1.00 mL, 0.0422 mmol) for 2 h. Evaporated to dryness. Toluene (15 mL) added and evaporated to dryness in vacuo twice to azeotrope of remaining TFA. Crude product taken straight on to next step. (3S)-5-(dimethylamino)-3-[[3-(5-methyl-1,2,4-oxadiazol-3-yl)benzoyl]amino]pentanoic acid (14 mg, 96%, 0.0404 mmol). HPLC/MS m/z: 347.2 [M+H]⁺, Rt (R): 0.70 min.

Example 162.7.: To (3S)-5-(dimethylamino)-3-[[3-(5-methyl-1,2,4-oxadiazol-3-yl)benzoyl]amino]pentanoic acid (14.00 mg, 0.0404 mmol) and 1-[fluoro(pyrrolidin-1-ium-1-ylidene)methyl]pyrrolidine hexafluorophosphate (23.00 mg, 0.0727 mmol) was added dry DCM (0.17 mL) and DIPEA (0.04 mL, 0.2223 mmol). The reaction was stirred at rt for 40 min [turned red] before propyl 2-amino-4-methyl-thiazole-5-carboxylate (28.33 mg, 0.1415 mmol) was added and the reaction mixture was heated at 60° C. behind a safety screen for 17 h. Temperature increased to 80° C. (CAUTION) as recommended in paper and stirred for further 6 h. Partially purified by reverse phase column chromatography (eluent: MeOH in water (+0.1% formic acid modifier in both)). Further purified by silica gel column chromatography (Eluent: 2-10% MeOH in DCM (+1% NH₃)) to afford propyl (S)-2-(5-(dimethylamino)-3-(3-(5-methyl-1,2,4-oxadiazol-3-yl)benzamido)pentanamido)-4-methylthiazole-5-carboxylate (1 mg, 5%, 0.0019 mmol) as a colourless glass. HPLC/MS m/z: 529.2 [M+H]⁺, Rt (S): 2.56 min. ¹H NMR (600 MHz, Methanol-d₄) δ 8.45 (t, J=1.7 Hz, 1H), 8.19 (dt, J=7.8, 1.4 Hz, 1H), 7.98-7.92 (m, 1H), 7.62 (t, J=7.8 Hz, 1H), 4.62-4.54 (m, 1H), 4.19 (t, J=6.6 Hz, 2H), 2.88-2.80 (m, 2H), 2.66 (s, 3H), 2.57 (s, 3H), 2.55-2.46 (m, 2H), 2.31 (s, 6H), 1.99-1.86 (m, 2H), 1.79-1.68 (m, 2H), 1.00 (t, J=7.4 Hz, 3H).

### Example 163: Propyl (S)-2-(6-amino-3-((7-(5-methyl-1,2,4-oxadiazol-3-yl)isoquinolin-1-yl)amino)hexanamido)-4-methylthiazole-5-carboxylate

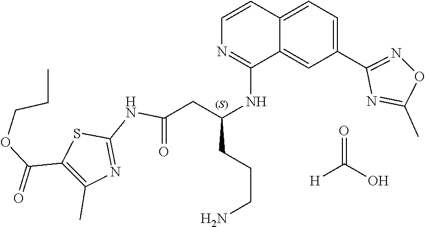

Example 163.1: To Fmoc-L-beta-Lys(Boc)-OH (1.00 g, 2.1343 mmol) under a nitrogen atmosphere was added anhydrous MeOH (21.34 mL) and the solution cooled in an ice bath before the dropwise addition of 2M TMS-Diazomethane in ether (7.5 mL) until the solution just retained a yellow colour. Reaction was stirred in ice bath for 4 h. Volatiles carefully removed in vacuo using teflon sleeve on ground glass joint to give methyl (3S)-6-(tert-butoxycarbonylamino)-3-(9H-fluoren-9-ylmethoxycarbonylamino)hexanoate (946 mg, 92%, 1.9603 mmol) as a colorless powder. HPLC/MS m/z: 505.2 [M+Na]⁺, Rt (P): 1.64 min.

Example 163.2.: To methyl (3S)-6-(tert-butoxycarbonylamino)-3-(9H-fluoren-9-ylmethoxycarbonylamino)hexanoate (300.00 mg, 0.6217 mmol) under nitrogen was added dry DMF (3.89 mL) followed by piperidine (193.97 uL, 1.9636 mmol). The reaction was stirred at rt for 6 h. The volatiles were removed in vacuo and the crude product purified by reverse phase column chromatography (eluent: 20-40% MeOH in water (+0.1% formic acid modifier in both)). Removal of the formic acid by ion exchange NH₂ column afforded methyl (3S)-3-amino-6-(tert-butoxycarbonylamino)-hexanoate (135 mg, 83%, 0.5186 mmol) as a colorless solid. HPLC/MS m/z: 261.2 [M+H]⁺, Rt (P): 0.85 min.

Example 163.3.: To 5-methyl-3-(2-oxidoisoquinolin-2-ium-7-yl)-1,2,4-oxadiazole [0](85.00 mg, 0.3741 mmol) under nitrogen atmosphere was added dry DCM (1.25 mL) and molecular sieves. After 10 min this solution was added to a methyl (3S)-3-amino-6-(tert-butoxycarbonylamino)hexanoate (107.12 mg, 0.4115 mmol), DIPEA (0.24 mL, 1.4028 mmol) followed by bromotri(pyrrolidino)phosphonium hexafluorophosphate (226.71 mg, 0.4863 mmol). The vial was sealed and stirred overnight under nitrogen atmosphere at rt. The volatiles were removed in vacuo and purified by reverse phase column chromatography (eluent: 20-80% MeOH in water (+0.1% formic acid modifier in both)) to methyl (3S)-6-(tert-butoxycarbonylamino)-3-[[7-(5-methyl-1,2,4-oxadiazol-3-yl)-1-isoquinolyl]amino]hexanoate (62 mg, 35%, 0.1320 mmol) as a yellow solid. HPLC/MS m/z: 470.2 [M+H]⁺, Rt (R): 1.12 min.

Example 163.4.: propyl (S)-2-(6-amino-3-((7-(5-methyl-1,2,4-oxadiazol-3-yl)isoquinolin-1-yl)amino)hexanamido)-4-methylthiazole-5-carboxylate formate was prepared by an analogous method to 0. Purification by prep-HPLC afforded propyl (S)-2-(6-amino-3-((7-(5-methyl-1,2,4-oxadiazol-3-yl)isoquinolin-1-yl)amino)hexanamido)-4-methylthiazole-5-carboxylate formate (1.63 mg, 0.0028 mmol). HPLC/MS m/z: 538.2 [M+H]⁺, Rt (S): 2.31 min. ¹H NMR (600 MHz, DMSO-d₆) 6 8.97 (s, 1H), 8.38 (broad s, 2H), 8.15 (d, J=8.4 Hz, 1H), 7.90 (d, J=5.6 Hz, 1H), 7.84 (d, J=8.5 Hz, 1H), 6.93 (d, J=5.7 Hz, 1H), 4.93-4.82 (m, 1H), 4.13 (t, J=6.5 Hz, 2H), 2.87 (dt, J=13.5, 6.8 Hz, 2H), 2.77 (dt, J=10.6, 5.5 Hz, 2H), 2.71 (s, 3H), 2.51 (s, 3H), 1.84-1.50 (m, 6H), 0.92 (t, J=7.3 Hz, 3H). (Note: 3×NH not observed).

The following examples were prepared in an analogous procedure:

### Example 164: (S)-6-amino-N-(5-(tert-butyl)-4-methylthiazol-2-yl)-3-((7-(5-methyl-1,2,4-oxadiazol-3-yl)isoquinolin-1-yl)amino)hexanamide formate

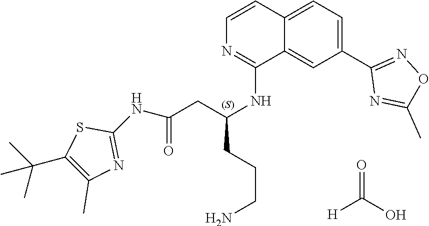

2.1 mg, colorless solid. HPLC/MS m/z: 508.2 [M+H]⁺, Rt (P): 1.29 min. ¹H NMR (600 MHz, DMSO-d₆) δ 8.97 (d, J=1.6 Hz, 1H), 8.41 (broad s, 1H), 8.15 (dd, J=8.5, 1.5 Hz, 1H), 7.93 (d, J=5.7 Hz, 1H), 7.84 (d, J=8.5 Hz, 1H), 7.66 (s, 1H), 6.94 (d, J=5.7 Hz, 1H), 4.85 (s, 1H), 2.82 (dd, J=14.8, 6.8 Hz, 1H), 2.78-2.65 (m, 6H), 2.28 (s, 3H), 1.80-1.54 (m, 4H), 1.33 (s, 9H). (Note: 3×NH not observed).

### Example 165: Cyclopentyl (S)-4-methyl-2-(3-((7-(5-methyl-1,2,4-oxadiazol-3-yl)isoquinolin-1-yl)amino)-6-(methylamino)hexanamido)thiazole-5-carboxylate

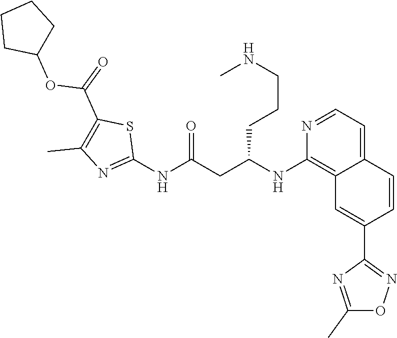 [SMILES: CNCCC[C@@H](CC(=O)Nc1nc(C)c(C(=O)OC2CCCC2)s1)Nc1nccc2ccc(-c3noc(C)n3)cc12]

7 mg (55%), colorless powder. HPLC/MS m/z: 578.3, [M+H]⁺, Rt (T): 1.29 min. ¹H NMR (500 MHz, Methanol-d₄) δ 9.29 (d, J=1.5 Hz, 1H), 8.54 (dd, J=8.4, 1.5 Hz, 1H), 8.07 (d, J=8.5 Hz, 1H), 7.75 (d, J=7.0 Hz, 1H), 7.34 (d, J=6.9 Hz, 1H), 5.27 (td, J=5.7, 4.6, 2.8 Hz, 1H), 4.80 (d, J=6.4 Hz, 1H), 3.18 (dd, J=6.4, 4.1 Hz, 2H), 3.09 (s, 2H), 2.70 (s, 3H), 2.70 (s, 3H), 2.51 (s, 3H), 2.09-1.99 (m, 2H), 1.97-1.84 (m, 4H), 1.79-1.69 (m, 4H), 1.69-1.59 (m, 2H).

### Example 166: 1,3-Difluoropropan-2-yl (S)-4-methyl-2-(3-((7-(5-methyl-1,2,4-oxadiazol-3-yl)isoquinolin-1-yl)amino)-6-(methylamino)hexanamido)thiazole-5-carboxylate

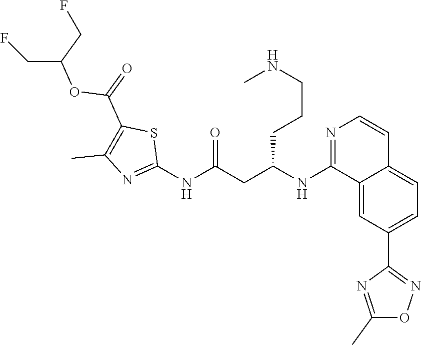 [SMILES: CNCCC[C@@H](CC(=O)Nc1nc(C)c(C(=O)OC(CF)CF)s1)Nc1nccc2ccc(-c3noc(C)n3)cc12]

10 mg (37%), colorless powder. HPLC/MS m/z: 588.2, [M+H]⁺, Rt (T): 1.06 min. ¹H NMR (600 MHz, DMSO-d₆) δ 8.95 (s, 1H), 8.14 (dd, J=8.5, 1.5 Hz, 1H), 7.90 (d, J=5.7 Hz, 1H), 7.83 (d, J=8.5 Hz, 1H), 6.91 (d, J=5.7 Hz, 1H), 5.46-5.31 (m, 1H), 4.89-4.81 (m, 1H), 4.77-4.69 (m, 2H), 4.69-4.59 (m, 2H), 2.82 (dd, J=14.9, 6.4 Hz, 1H), 2.76-2.67 (m, 4H), 2.63-2.55 (m, 2H), 2.52 (s, 3H), 2.31 (s, 3H), 1.82-1.66 (m, 2H), 1.62-1.46 (m, 2H). (Note: 3×NH not observed).

### Example 167: Isopropyl (S)-4-methyl-2-(3-((7-(5-methyl-1,2,4-oxadiazol-3-yl)isoquinolin-1-yl)amino)-6-(methylamino)hexanamido)thiazole-5-carboxylate

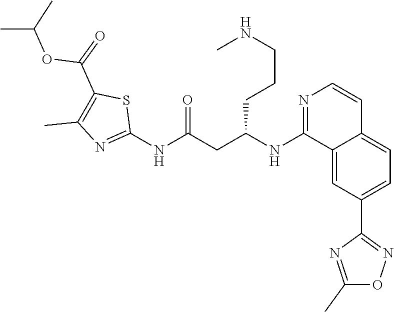 [SMILES: CNCCC[C@@H](CC(=O)Nc1nc(C)c(C(=O)OC(C)C)s1)Nc1nccc2ccc(-c3noc(C)n3)cc12]

20 mg (79%), colorless powder. HPLC/MS m/z: 552.3, [M+H]⁺, Rt (T): 1.15 min. ¹H NMR (600 MHz, DMSO-d₆) δ 8.94 (s, 1H), 8.13 (dd, J=8.5, 1.5 Hz, 1H), 7.90 (d, J=5.7 Hz, 1H), 7.82 (d, J=8.5 Hz, 1H), 6.93-6.89 (m, 1H), 5.02 (hept, J=6.2 Hz, 1H), 4.88-4.80 (m, 1H), 2.83 (dd, J=14.8, 6.6 Hz, 1H), 2.78-2.67 (m, 4H), 2.54-2.51 (m, 2H), 2.49 (s, 3H), 2.27 (s, 3H), 1.81-1.64 (m, 2H), 1.59-1.44 (m, 2H), 1.25 (d, J=6.3 Hz, 6H). (Note: 3×NH not observed).

### Example 168: tert-Butyl (S)-4-methyl-2-(3-((7-(5-methyl-1,2,4-oxadiazol-3-yl)isoquinolin-1-yl)amino)-6-(methylamino)hexanamido)thiazole-5-carboxylate

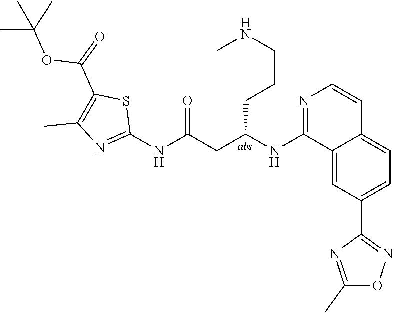 [SMILES: CNCCC[C@@H](CC(=O)Nc1nc(C)c(C(=O)OC(C)(C)C)s1)Nc1nccc2ccc(-c3noc(C)n3)cc12]

13 mg (38%), colorless powder. HPLC/MS m/z: 566.3 [M+H]⁺, Rt (Q): 2.53 min. ¹H NMR (600 MHz, DMSO-d₆) δ 8.96 (d, J=1.6 Hz, 1H), 8.15 (dd, J=8.5, 1.5 Hz, 1H), 7.91 (d, J=5.7 Hz, 1H), 7.83 (d, J=8.5 Hz, 1H), 6.95-6.89 (m, 1H), 4.90-4.80 (m, 1H), 2.84 (dd, J=14.9, 6.7 Hz, 1H), 2.77-2.73 (m, 1H), 2.72 (s, 3H), 2.55-2.51 (m, 2H), 2.49 (s, 3H), 2.27 (s, 3H), 1.82-1.64 (m, 2H), 1.61-1.51 (m, 2H), 1.49 (s, 9H). (Note: 3×NH not observed).

### Example 169: (S)-4-methyl-2-(3-((7-(5-methyl-1,2,4-oxadiazol-3-yl)isoquinolin-1-yl)amino)-6-(methylamino)hexanamido)thiazole-5-carboxylic acid

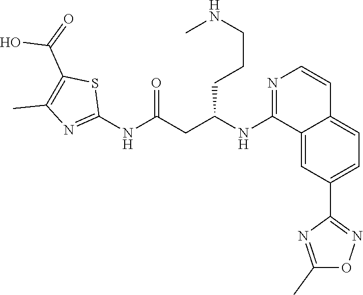 [SMILES: CNCCC[C@@H](CC(=O)Nc1nc(C)c(C(=O)O)s1)Nc1nccc2ccc(-c3noc(C)n3)cc12]

Removal of Boc protecting group of tert-butyl 2-[[(3S)-6-[tert-butoxycarbonyl-(methyl)amino]-3-[[7-(5-methyl-1,2,4-oxadiazol-3-yl)-1-isoquinolyl]amino]-hexanoyl]amino]-4-methyl-thiazole-5-carboxylate using TFA in 0 also afforded (S)-4-methyl-2-(3-((7-(5-methyl-1,2,4-oxadiazol-3-yl)isoquinolin-1-yl)amino)-6-(methylamino)hexanamido)thiazole-5-carboxylic acid (50 mg, 80%, 0.0893 mmol) as a colorless powder. HPLC/MS m/z: 510.2, [M+H]⁺, Rt (Q): 2.10 min. ¹H NMR (600 MHz, Methanol-d₄) δ 9.18-9.14 (m, 1H), 8.45 (dd, J=8.4, 1.5 Hz, 1H), 7.99 (d, J=8.4 Hz, 1H), 7.79 (d, J=6.6 Hz, 1H), 7.24 (d, J=6.7 Hz, 1H), 4.78-4.73 (m, 1H), 3.14-3.01 (m, 4H), 2.70 (s, 3H), 2.69 (s, 3H), 2.53 (s, 3H), 2.05-1.95 (m, 2H), 1.93-1.80 (m, 2H).

### Example 170: 3,3-Difluorocyclopentyl 4-methyl-2-((S)-3-((7-(5-methyl-1,2,4-oxadiazol-3-yl)isoquinolin-1-yl)amino)-6-(methylamino)hexanamido)thiazole-5-carboxylate

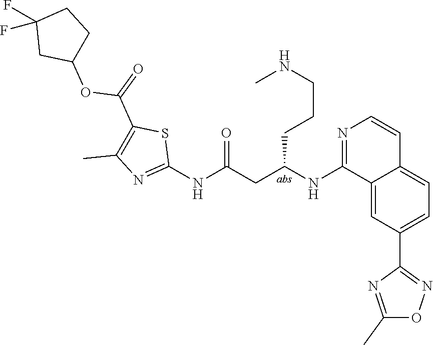 [SMILES: CNCCC[C@@H](CC(=O)Nc1nc(C)c(C(=O)OC2CCC(F)(F)C2)s1)Nc1nccc2ccc(-c3noc(C)n3)cc12]

67 mg (74%), cream-coloured powder. HPLC/MS m/z: 614.2, [M+H]⁺, Rt (Q): 2.46 min. ¹H NMR (600 MHz, DMSO-d₆) δ 8.95 (s, 1H), 8.14 (dd, J=8.5, 1.5 Hz, 1H), 7.91 (d, J=5.7 Hz, 1H), 7.83 (d, J=8.5 Hz, 1H), 6.92 (d, J=5.7 Hz, 1H), 5.33-5.28 (m, 1H), 4.88-4.81 (m, 1H), 2.83 (dd, J=15.0, 6.4 Hz, 1H), 2.77-2.68 (m, 4H), 2.63-2.52 (m, 3H), 2.51 (s, 3H), 2.33-2.13 (m, 7H), 1.98-1.90 (m, 1H), 1.83-1.65 (m, 2H), 1.61-1.45 (m, 2H).

### Example 171: (S)-N-(5-(tert-butyl)-4-methylthiazol-2-yl)-3-((7-(5-methyl-1,2,4-oxadiazol-3-yl)isoquinolin-1-yl)amino)-6-(methylamino)hexanamide

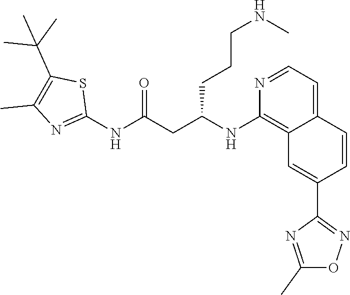 [SMILES: CNCCC[C@@H](CC(=O)Nc1nc(C)c(C(C)(C)C)s1)Nc1nccc2ccc(-c3noc(C)n3)cc12]

Example 171.1.: 5-methyl-3-(2-oxidoisoquinolin-2-ium-7-yl)-1,2,4-oxadiazole [0](120 mg, 0.5281 mmol) was dissolved/suspended in dry DCM (1.76 mL) and added to a RBF containing tert-butyl (3S)-3-amino-6-[tert-butoxycarbonyl(methyl)amino]hexanoate (183.83 mg, 0.5809 mmol), quickly followed by DIPEA (0.34 mL, 1.9805 mmol). To this solution was added PyBroP (320.06 mg, 0.6866 mmol) and the mixture was stirred overnight at rt. Purified by reverse phase column chromatography (eluent: 20-80% MeOH in water (+0.1% formic acid)) to afford tert-butyl (3S)-6-[tert-butoxycarbonyl(methyl)amino]-3-[[7-(5-methyl-1,2,4-oxadiazol-3-yl)-1-isoquinolyl]amino]hexanoate a colorless solid. HPLC/MS m/z: 526.34, [M+H]⁺, Rt (P): 1.44 min.

Example 171.2.: tert-Butyl (3S)-6-[tert-butoxycarbonyl(methyl)amino]-3-[[7-(5-methyl-1,2,4-oxadiazol-3-yl)-1-isoquinolyl]amino]hexanoate (160.00 mg, 0.3044 mmol) and potassium hydroxide (136.63 mg, 2.4351 mmol) were dissolved in THE (1.79 mL) at rt. Few drops of water (enough to dissolve KOH) were added and the resulting solution was stirred at 50° C. for 3.5 h. 8 eq of KOH were added. Few drops of MeOH were added and the resulting solution was stirred at 50° C. overnight. LCMS showed 65% conversion. 4 eq of KOH followed by few drops of MeOH and water were added and the solution was stirred for 4 h at 60 0° C. Half the reaction mixture was purified by reverse phase column chromatography (eluent: 20-80% MeOH in water (+0.1% formic acid as buffer in both)) to afford (3S)-6-[tert-butoxycarbonyl(methyl)amino]-3-[[7-(5-methyl-1,2,4-oxadiazol-3-yl)-1-isoquinolyl]amino]hexanoic acid; formic acid (50 mg, 32%, 0.0970 mmol) as a colorless solid. HPLC/MS m/z: 470.26, [M+H]⁺, Rt (P): 1.34 min.

Example 171.3.: To a mixture of (3S)-6-[tert-butoxycarbonyl(methyl)amino]-3-[[7-(5-methyl-1,2,4-oxadiazol-3-yl)-1-isoquinolyl]amino]hexanoic acid (71.46 mg, 0.1522 mmol), EDC.HCl (58.35 mg, 0.3044 mmol), HOBt (41.13 mg, 0.3044 mmol) and 5-tert-butyl-4-methyl-thiazol-2-ylamine (31.10 mg, 0.1826 mmol) DMF (0.76 mL) was added under nitrogen atmosphere. The resulting solution was stirred for 24 h at 60 0° C. 1 eq of EDC.HCl and 1 eq of HOBt were added and the resulting solution was stirred at 70° C. for 36 h. Purified by reverse phase column chromatography (eluent: 0-90% MeOH in water (+0.1% formic acid)) to afford tert-butyl N-[(4S)-6-[(5-tert-butyl-4-methyl-thiazol-2-yl)amino]-4-[[7-(5-methyl-1,2,4-oxadiazol-3-yl)-1-isoquinolyl]amino]-6-oxo-hexyl]-N-methyl-carbamate; formic acid (11.5 mg, 11%, 0.0172 mmol) as a colorless powder. HPLC/MS m/z: 622.31, [M+H]⁺, Rt (R): 1.55 min.

Example 171.4.: To a solution of tert-butyl N-[(4S)-6-[(5-tert-butyl-4-methyl-thiazol-2-yl)amino]-4-[[7-(5-methyl-1,2,4-oxadiazol-3-yl)-1-isoquinolyl]amino]-6-oxo-hexyl]-N-methyl-carbamate formate (12.35 mg, 0.0185 mmol) in dioxane (0.10 mL) was added HCl dioxane (4 M) (0.02 mL, 0.0767 mmol). The resulting solution was stirred at rt for 5.5 h. Volatiles were removed under reduced pressure and the residue was purified by semi-prep HPLC. Further purification by ion exchange SCX-II column chromatography, washing with MeOH before eluting with 2M NH₃ in MeOH afforded (3S)-N-(5-tert-butyl-4-methyl-thiazol-2-yl)-6-(methylamino)-3-[[7-(5-methyl-1,2,4-oxadiazol-3-yl)-1-isoquinolyl]amino]hexanamide (4 mg, 42%, 0.0077 mmol) as a colourless oil. HPLC/MS m/z: 522.26, [M+H]⁺, Rt (Q): 2.46 min. ¹H NMR (600 MHz, DMSO-d₆) δ 8.96 (d, J=1.5 Hz, 1H), 8.15 (dd, J=8.5, 1.5 Hz, 1H), 7.93 (d, J=5.7 Hz, 1H), 7.84 (d, J=8.5 Hz, 1H), 7.68-7.63 (m, 1H), 6.93 (d, J=5.7 Hz, 1H), 4.82 (d, J=9.0 Hz, 1H), 2.81 (dd, J=14.7, 6.7 Hz, 1H), 2.72 (s, 3H), 2.68 (dd, J=14.7, 6.8 Hz, 1H), 2.55-2.51 (m, 2H), 2.29 (s, 3H), 2.27 (s, 3H), 1.78-1.71 (m, 1H), 1.65 (ddd, J=13.6, 9.7, 5.0 Hz, 1H), 1.60-1.46 (m, 2H), 1.34 (s, 9H). (Note: 2×NH not observed).

The following examples were prepared by an analogous procedure:

### Example 172: Propyl (S)-4-methyl-2-(3-((7-(5-methyl-1,2,4-oxadiazol-3-yl)isoquinolin-1-yl)amino)-6-(methylamino)hexanamido)thiazole-5-carboxylate

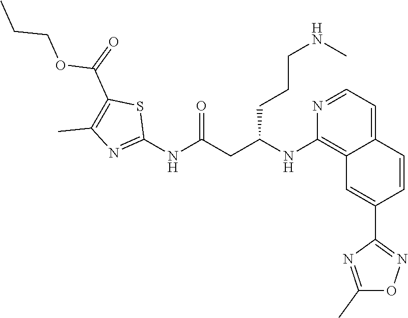 [SMILES: CCCOC(=O)c1sc(NC(=O)C[C@H](CCCNC)Nc2nccc3ccc(-c4noc(C)n4)cc23)nc1C]

(7.2 mg, 43%, 0.0131 mmol) as a colorless solid. HPLC/MS m/z: 552.24, [M+H]⁺, Rt (Q): 2.48 min. ¹H NMR (600 MHz, DMSO-d₆) δ 8.96 (d, J=1.5 Hz, 1H), 8.15 (dd, J=8.4, 1.5 Hz, 1H), 7.91 (d, J=5.7 Hz, 1H), 7.83 (d, J=8.5 Hz, 1H), 6.92 (d, J=5.7 Hz, 1H), 4.88-4.81 (m, 1H), 4.13 (t, J=6.5 Hz, 2H), 2.84 (dd, J=14.9, 6.5 Hz, 1H), 2.75 (dd, J=15.1, 7.0 Hz, 1H), 2.72 (s, 3H), 2.56 (td, J=7.1, 2.5 Hz, 2H), 2.52 (s, 3H), 2.30 (s, 3H), 1.79 (ddt, J=18.6, 9.1, 4.6 Hz, 1H), 1.73-1.67 (m, 1H), 1.65 (q, J=7.1 Hz, 2H), 1.61-1.46 (m, 2H), 0.93 (t, J=7.4 Hz, 3H). (Note: 3×NH not observed).

### Example 173: Propyl (R)-2-(2-amino-3-(3-(5-methyl-1,2,4-oxadiazol-3-yl)benzamido)propanamido)-4-methylthiazole-5-carboxylate formate

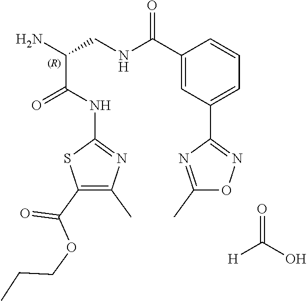

Example 173.1.: To 3-(5-methyl-1,2,4-oxadiazol-3-yl)benzoic acid (67.44 mg, 0.3303 mmol), (R)-ethyl 3-amino-2-(CBZ-amino)propanoate HCl (100.00 mg, 0.3303 mmol) and HATU (125.59 mg, 0.3303 mmol) was added dry DMF (1.65 mL) and DIPEA (0.17 mL, 0.9909 mmol). Stirred at rt overnight. Purified by reverse phase column chromatography (eluent: 30-100% MeOH in water (+0.1% formic acid modifier in both) to afford ethyl (2R)-2-(benzyloxycarbonylamino)-3-[[3-(5-methyl-1,2,4-oxadiazol-3-yl)benzoyl]amino]propanoate (121 mg, 81%, 0.2674 mmol) as a colorless powder. HPLC/MS m/z: 453.2 [M+H]⁺, Rt (P): 1.47 min.

Example 173.2.: Ethyl (2R)-2-(benzyloxycarbonylamino)-3-[[3-(5-methyl-1,2,4-oxadiazol-3-yl)benzoyl]amino]propanoate (118.00 mg, 0.2608 mmol) was dissolved in THF (1.3 mL) and water (1.3 mL) was added. Lithium hydroxide hydrate (131.32 mg, 3.1296 mmol) was added and then additional THF (0.63 mL). Stirred at rt for 4 h before the volatiles were removed in vacuo. Purified by reverse phase column chromatography (eluent: 20-80% MeOH in water (+0.1% formic acid modifier in both)). Product dissolved in CHCl₃ and filtered through celite to remove inorganics. Concentrating in vacuo afforded (2R)-2-(benzyloxycarbonylamino)-3-[[3-(5-methyl-1,2,4-oxadiazol-3-yl)benzoyl]amino]propanoic acid (100 mg, 90%, 0.2356 mmol). HPLC/MS m/z: 425.1 [M+H]⁺, Rt (T): 1.24 min.

Example 173.3.: To propyl 2-amino-4-methyl-thiazole-5-carboxylate (50.87 mg, 0.2540 mmol), HOBt (62.40 mg, 0.4618 mmol), and 3-(ethyliminomethyleneamino)-N,N-dimethyl-propan-1-amine hydrochloride (88.53 mg, 0.4618 mmol) under nitrogen atmosphere was added (2R)-2-(benzyloxycarbonylamino)-3-[[3-(5-methyl-1,2,4-oxadiazol-3-yl)benzoyl]amino]propanoic acid (98.00 mg, 0.2309 mmol) in DMF (1.15 mL) and the reaction mixture was stirred at 60° C. for 20 h. Purified directly by reverse phase column chromatography (eluent: 30-100% MeOH in water (+0.1% Formic acid modifier in both)) to afford propyl 2-[[(2R)-2-(benzyloxycarbonylamino)-3-[[3-(5-methyl-1,2,4-oxadiazol-3-yl)benzoyl]amino]propanoyl]amino]-4-methyl-thiazole-5-carboxylate (42 mg, 30%, 0.0692 mmol) as an off-white powder. HPLC/MS m/z: 607.2 [M+H]⁺, Rt (Q): 3.20 min.

Example 173.4.: A 1.9 M solution of HBr in AcOH was made by mixing 0.33 mL of 33% wt HBr in AcOH [-5.7 M] and 0.66 mL of AcOH (fumes). 0.5 mL of this solution was added to propyl 2-[[(2R)-2-(benzyloxycarbonylamino)-3-[[3-(5-methyl-1,2,4-oxadiazol-3-yl)benzoyl]amino]propanoyl]amino]-4-methyl-thiazole-5-carboxylate (22.00 mg, 0.0363 mmol) which was stirred at rt for 75 min. Ether (3 mL) was added and the white precipitate was removed by filtration. Purified by reverse phase column chromatography (eluent: 20-100% MeOH in water (+0.1% formic acid modifier in both)) afforded propyl (R)-2-(2-amino-3-(3-(5-methyl-1,2,4-oxadiazol-3-yl)benzamido)propanamido)-4-methylthiazole-5-carboxylate formate (1.7 mg, 9%, 0.0033 mmol) as a colorless powder. HPLC/MS m/z: 473.2 [M+H]⁺, Rt (U): 2.44 min. ¹H NMR (600 MHz, DMSO-d₆) δ 8.79 (t, J=5.8 Hz, 1H), 8.43 (d, J=1.8 Hz, 1H), 8.25 (s, 1H), 8.12 (dt, J=7.8, 1.4 Hz, 1H), 8.01 (dt, J=7.8, 1.5 Hz, 1H), 7.65 (t, J=7.8 Hz, 1H), 4.14 (t, J=6.5 Hz, 2H), 3.76 (t, J=6.4 Hz, 1H), 3.57 (td, J=6.1, 2.4 Hz, 2H), 2.68 (s, 3H), 2.52 (s, 3H), 1.66 (q, J=7.0 Hz, 2H), 0.93 (t, J=7.4 Hz, 3H). (Note: 3×NH not observed).

### Example 174: Propyl (S)-2-(2-amino-3-((7-(5-methyl-1,2,4-oxadiazol-3-yl)isoquinolin-1-yl)amino)propanamido)-4-methylthiazole-5-carboxylate

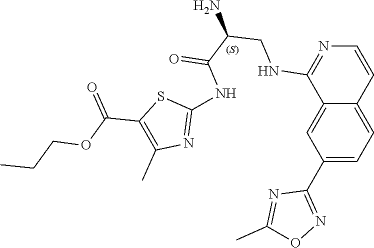

Example 174.1.: 5-Methyl-3-(2-oxidoisoquinolin-2-ium-7-yl)-1,2,4-oxadiazole [Example 96](253 mg, 1.1135 mmol) was dissolved/suspended in DCM (3.71 mL) and tert-butyl (2S)-3-amino-2-(tert-butoxycarbonylamino)propanoate (362.33 mg, 1.3918 mmol) added, quickly followed by DIPEA (0.73 mL, 4.1755 mmol). To this solution was added PyBroP (674.80 mg, 1.4475 mmol) and the mixture stirred at rt overnight. Purified by silica gel column chromatography (eluent: 1-10% EtOH in DCM) to afford tert-butyl (2S)-2-(tert-butoxycarbonylamino)-3-[[7-(5-methyl-1,2,4-oxadiazol-3-yl)-1-isoquinolyl]amino]propanoate (230 mg, 44%, 0.4899 mmol). HPLC/MS m/z: 470.2 [M+H]⁺, Rt (P): 1.41 min.

Example 174.2.: Propyl (S)-2-(2-amino-3-((7-(5-methyl-1,2,4-oxadiazol-3-yl)isoquinolin-1-yl)amino)propanamido)-4-methylthiazole-5-carboxylate was prepared by an analogous method to 0 from tert-butyl (2S)-2-(tert-butoxycarbonylamino)-3-[[7-(5-methyl-1,2,4-oxadiazol-3-yl)-1-isoquinolyl]amino]propanoate. Purification by semi-prep HPLC afforded propyl (S)-2-(2-amino-3-((7-(5-methyl-1,2,4-oxadiazol-3-yl)isoquinolin-1-yl)amino)propanamido)-4-methylthiazole-5-carboxylate (5 mg, 0.0101 mmol). HPLC/MS m/z: 496.2 [M+H]⁺, Rt (U): 2.78 min. ¹H NMR (600 MHz, DMSO-d₆) δ 8.85 (s, 1H), 8.20-8.12 (m, 1H), 8.01 (s, 1H), 7.87 (d, J=8.5 Hz, 1H), 7.84 (d, J=5.7 Hz, 1H), 7.00 (d, J=5.8 Hz, 1H), 6.55 (broad s, 2H), 4.24 (t, J=5.5 Hz, 1H), 4.18-4.10 (m, 2H), 4.08-4.02 (m, 1H), 3.92 (dd, J=14.0, 5.4 Hz, 1H), 2.69 (s, 3H), 2.53 (d, J=9.3 Hz, 3H), 1.65 (sext, J=7.1 Hz, 2H), 0.92 (t, J=7.4 Hz, 3H). (Note: 1×NH not observed).

### Example 175: Propyl (S)-2-(2-(dimethylamino)-3-(3-(5-methyl-1,2,4-oxadiazol-3-yl)benzamido)propanamido)-4-methylthiazole-5-carboxylate formate

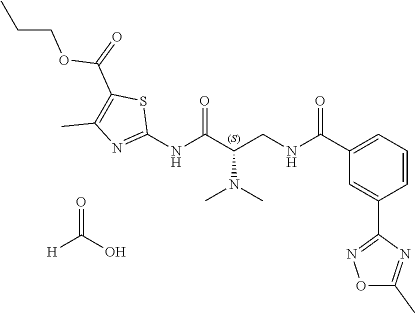

Example 175.1: H-Dap(Boc)-OMe HCl (250.00 mg, 0.9815 mmol) was suspended in N,N-Diisopropylethylamine (1.66 mL, 9.4731 mmol) and iodomethane (0.18 mL, 2.9445 mmol) was added. The mixture was stirred at rt overnight. Further 1.5 eq of Mel added and stirred at rt. Purified by reverse phase column chromatography (eluent: 5-15% MeOH in water (+0.1% formic acid modifier in both)) to afford methyl (S)-3-((tert-butoxycarbonyl)amino)-2-(dimethylamino)propanoate formate (115 mg, 40%, 0.3934 mmol). HPLC/MS m/z: 247.2 [M+H]⁺, Rt (Q): 0.54 min.

Example 175.2.: Methyl (S)-3-((tert-butoxycarbonyl)amino)-2-(dimethylamino)-propanoate (110.00 mg, 0.4466 mmol) was dissolved in dry MeOH (0.25 mL) and 4M HCl in dioxane (0.11 mL, 0.4466 mmol) was added. Stirred for 5 h at rt before volatiles were carefully removed in vacuo to afford methyl (S)-3-amino-2-(dimethyl-amino)propanoate hydrochloride which was used without purification. HPLC/MS m/z: 147.1 [M+H]⁺, Rt (Q): 0.15 min.

Example 175.3.: Methyl (S)-3-amino-2-(dimethylamino)propanoate hydrochloride was utilised in an analogous synthesis to 0 to afford propyl (S)-2-(2-(dimethylamino)-3-(3-(5-methyl-1,2,4-oxadiazol-3-yl)benzamido)propanamido)-4-methylthiazole-5-carboxylate formate (17 mg, 16%, 0.0311 mmol) as a colorless powder. HPLC/MS m/z: 501.2 [M+H]⁺, Rt (Q): 2.49 min. ¹H NMR (600 MHz, DMSO-d₆) δ 12.52 (s, 1H), 8.81 (t, J=5.7 Hz, 1H), 8.44 (d, J=1.7 Hz, 1H), 8.17 (s, 1H), 8.12 (dt, J=7.7, 1.4 Hz, 1H), 8.01 (dt, J=7.9, 1.4 Hz, 1H), 7.64 (t, J=7.8 Hz, 1H), 4.16 (t, J=6.5 Hz, 2H), 3.76 (dd, J=7.7, 6.3 Hz, 1H), 3.67 (dt, J=12.4, 6.1 Hz, 1H), 3.55 (ddd, J=13.2, 7.8, 5.5 Hz, 1H), 2.68 (s, 3H), 2.53 (s, 3H), 2.31 (s, 6H), 1.68 (sext, J=7.1 Hz, 2H), 0.95 (t, J=7.4 Hz, 3H).

### Example 176: Propyl (R)-2-(2-hydroxy-3-((7-(5-methyl-1,2,4-oxadiazol-3-yl)isoquinolin-1-yl)amino)propanamido)-4-methylthiazole-5-carboxylate

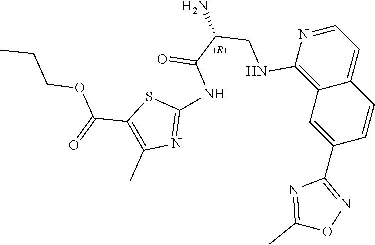

Example 176.1.: To (2R)-3-(tert-butoxycarbonylamino)-2-hydroxy-propanoic acid (182.00 mg, 0.8869 mmol), propyl 2-amino-4-methyl-thiazole-5-carboxylate (195.37 mg, 0.9756 mmol), HOBt (239.67 mg, 1.7738 mmol) and EDC.HCl (340.04 mg, 1.7738 mmol) was added DMF (4.43 mL), the vial capped, and the reaction mixture heated at 50° C. for 16 h. The volatiles were removed in vacuo and the resulting orange gum purified by reverse phase column chromatography (eluent: 30-100% MeOH in water (+0.1% formic acid modifier in both)) to afford propyl 2-[[(2R)-3-(tert-butoxycarbonylamino)-2-hydroxy-propanoyl]amino]-4-methyl-thiazole-5-carboxylate (230 mg, 67%, 0.5936 mmol) as a colorless powder. HPLC/MS m/z: 388.2 [M+H]⁺, Rt (V): 1.57 min.

Example 176.2.: To propyl 2-[[(2R)-3-(tert-butoxycarbonylamino)-2-hydroxy-propanoyl]amino]-4-methyl-thiazole-5-carboxylate (220.00 mg, 0.5678 mmol) was added propanol (2.03 mL) followed by 4M HCl in dioxane (0.18 mL). Stirred at rt for 1.5 h before further 4M HCl in dioxane (1.8 mL) was added. Stirred for 1 h 40 min at rt before the volatiles were removed in vacuo. Purification by ion exchange SCX-II column chromatography, washing with MeOH before elution with 2M NH₃ in MeOH, upon concentration afforded propyl 2-[[(2R)-3-amino-2-hydroxy-propanoyl]amino]-4-methyl-thiazole-5-carboxylate (158 mg, 97%, 0.5499 mmol) as a light-yellow powder. HPLC/MS m/z: 288.1 [M+H]⁺, Rt (U): 1.96 min.

Example 176.3.: 5-methyl-3-(2-oxidoisoquinolin-2-ium-7-yl)-1,2,4-oxadiazole [0](65.00 mg, 0.2861 mmol) and propyl 2-[[(2R)-3-amino-2-hydroxy-propanoyl]amino]-4-methyl-thiazole-5-carboxylate (82.20 mg, 0.2861 mmol) were suspended in dry DCM (2.0 mL). PyBroP (173.37 mg, 0.3719 mmol) and DIPEA (186.85 uL, 1.0727 mmol) were added and a nitrogen atmosphere introduced. Stirred at rt for 22 h. Diluted with DCM/MeOH and purified by silica gel column chromatography (eluent: 5-70% EtOAc in cyclohexane) to afford propyl (R)-2-(2-hydroxy-3-((7-(5-methyl-1,2,4-oxadiazol-3-yl)isoquinolin-1-yl)amino)propanamido)-4-methylthiazole-5-carboxylate (14 mg, 10%, 0.0282 mmol) as an off-white powder. HPLC/MS m/z: 497.2 [M+H]⁺, Rt (Q): 2.65 min.¹H NMR (600 MHz, DMSO-d₆) δ 12.30 (s, 1H), 8.89 (d, J=1.7 Hz, 1H), 8.16 (dd, J=8.5, 1.6 Hz, 1H), 8.06 (t, J=5.7 Hz, 1H), 7.91 (d, J=5.7 Hz, 1H), 7.86 (d, J=8.5 Hz, 1H), 6.99 (d, J=5.8 Hz, 1H), 6.43 (s, 1H), 4.58 (t, J=5.6 Hz, 1H), 4.15 (t, J=6.5 Hz, 2H), 3.89-3.78 (m, 2H), 2.69 (s, 3H), 2.52 (s, 3H), 1.67 (sext, J=7.1 Hz, 2H), 0.93 (t, J=7.4 Hz, 3H).

The following examples were prepared by an analogous procedure:

### Example 177: tert-Butyl (R)-2-(2-hydroxy-3-((7-(5-methyl-1,2,4-oxadiazol-3-yl)isoquinolin-1-yl)amino)propanamido)-4-methylthiazole-5-carboxylate

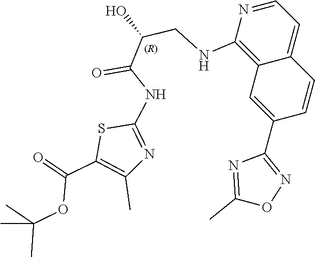

Prepared using tert-butyl 2-amino-4-methyl-thiazole-5-carboxylate [Example 110.1] in an analogous procedure to Example 176 to afford tert-butyl (R)-2-(2-hydroxy-3-((7-(5-methyl-1,2,4-oxadiazol-3-yl)isoquinolin-1-yl)amino)propanamido)-4-methylthiazole-5-carboxylate (12 mg, 10%, 0.0233 mmol) as a colorless powder. HPLC/MS m/z: 511.2 [M+H]⁺, Rt (U): 2.78 min. ¹H NMR (600 MHz, Chloroform-d) δ 10.28 (s, 1H), 9.04 (s, 1H), 8.49-8.45 (m, 1H), 8.28 (dd, J=8.5, 1.5 Hz, 1H), 7.93 (d, J=6.0 Hz, 1H), 7.79 (d, J=8.5 Hz, 1H), 7.08 (dd, J=6.0, 0.9 Hz, 1H), 6.08 (t, J=5.6 Hz, 1H), 4.61 (dd, J=4.4, 2.2 Hz, 1H), 4.20 (ddd, J=14.8, 4.8, 2.2 Hz, 1H), 4.12 (ddd, J=14.8, 6.4, 4.4 Hz, 1H), 2.69 (s, 3H), 2.54 (s, 3H), 1.56 (s, 9H).

### Example 178: Propyl (S)-2-(2-hydroxy-3-((7-(5-methyl-1,2,4-oxadiazol-3-yl)isoquinolin-1-yl)amino)propanamido)-4-methylthiazole-5-carboxylate

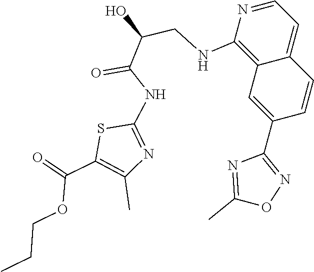 [SMILES: CCCOC(=O)c1sc(NC(=O)[C@@H](O)CNc2nccc3ccc(-c4noc(C)n4)cc23)nc1C]

Prepared using N-tert-Butyloxycarbonyl-(S)-isoserine in an analogous procedure to Example 176 to afford propyl (S)-2-(2-hydroxy-3-((7-(5-methyl-1,2,4-oxadiazol-3-yl)isoquinolin-1-yl)amino)propanamido)-4-methylthiazole-5-carboxylate (26 mg, 25%, 0.0513 mmol) as a colorless powder. HPLC/MS m/z: 497.2 [M+H]⁺, Rt (Q): 2.62 min. ¹H NMR (600 MHz, DMSO-d₆) δ 12.49-12.11 (m, 1H), 8.89 (d, J=1.6 Hz, 1H), 8.16 (dd, J=8.5, 1.5 Hz, 1H), 8.06 (t, J=5.6 Hz, 1H), 7.90 (d, J=5.8 Hz, 1H), 7.86 (d, J=8.5 Hz, 1H), 6.99 (d, J=5.8 Hz, 1H), 6.44 (s, 1H), 4.58 (t, J=5.6 Hz, 1H), 4.15 (t, J=6.5 Hz, 2H), 3.89-3.78 (m, 2H), 2.69 (s, 3H), 2.52 (s, 3H), 1.67 (q, J=7.0 Hz, 2H), 0.93 (t, J=7.4 Hz, 3H).

### Example 179: (S)-2-hydroxy-N-(5-isobutyl-4-methylthiazol-2-yl)-3-((7-(5-methyl-1,2,4-oxadiazol-3-yl)isoquinolin-1-yl)amino)propenamide

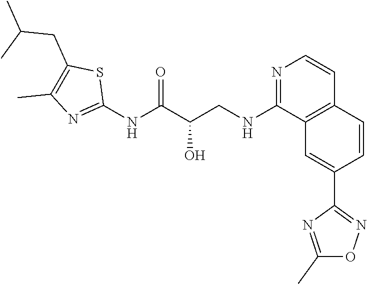 [SMILES: Cc1nc(-c2ccc3ccnc(NC[C@H](O)C(=O)Nc4nc(C)c(CC(C)C)s4)c3c2)no1]

6.8 mg (12%). HPLC/MS m/z: 467.19, [M+H]⁺, Rt (U): 2.75 min. ¹H NMR (600 MHz, Chloroform-d) δ 10.10 (s, 1H), 8.99 (s, 1H), 8.48 (d, J=1.6 Hz, 1H), 8.26 (dd, J=8.5, 1.5 Hz, 1H), 7.92 (d, J=5.9 Hz, 1H), 7.77 (d, J=8.5 Hz, 1H), 7.05 (d, J=6.0 Hz, 1H), 6.15 (t, J=5.6 Hz, 1H), 4.58 (dd, J=4.4, 2.2 Hz, 1H), 4.19 (ddd, J=14.8, 4.9, 2.3 Hz, 1H), 4.12 (ddd, J=14.7, 6.5, 4.4 Hz, 1H), 2.67 (s, 3H), 2.48 (dd, J=7.1, 1.8 Hz, 2H), 2.15 (s, 3H), 1.85-1.74 (m, 1H), 0.90 (dd, J=8.5, 6.6 Hz, 6H).

### Example 180: tert-Butyl (S)-2-(2-hydroxy-3-((7-(5-methyl-1,2,4-oxadiazol-3-yl)isoquinolin-1-yl)amino)propanamido)-4-methylthiazole-5-carboxylate

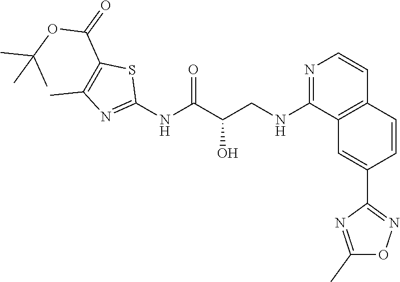 [SMILES: Cc1nc(-c2ccc3ccnc(NC[C@H](O)C(=O)Nc4nc(C)c(C(=O)OC(C)(C)C)s4)c3c2)no1]

10 mg (25%). HPLC/MS m/z: 511.18, [M+H]⁺, Rt (U): 2.79 min. ¹H NMR (600 MHz, Chloroform-d) δ 10.29 (s, 1H), 9.19-8.95 (m, 1H), 8.48 (s, 1H), 8.28 (dd, J=8.5, 1.5 Hz, 1H), 7.93 (d, J=6.0 Hz, 1H), 7.79 (d, J=8.5 Hz, 1H), 7.07 (d, J=6.0 Hz, 1H), 6.12 (t, J=5.7 Hz, 1H), 4.61 (dd, J=4.4, 2.2 Hz, 1H), 4.20 (ddd, J=14.7, 4.8, 2.2 Hz, 1H), 4.12 (ddd, J=14.8, 6.4, 4.4 Hz, 1H), 2.68 (s, 3H), 2.54 (s, 3H), 1.53 (s, 9H).

### Example 181: tert-Butyl (S)-2-(2-hydroxy-3-((7-(1-methyl-1 H-pyrazol-4-yl)isoquinolin-1-yl)amino)propanamido)-4-methylthiazole-5-carboxylate

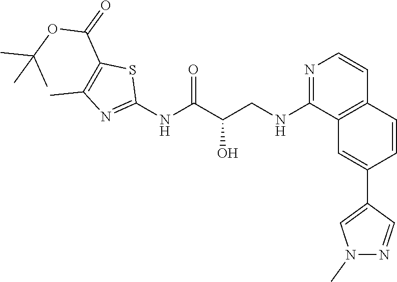 [SMILES: Cc1nc(NC(=O)[C@@H](O)CNc2nccc3ccc(-c4cnn(C)c4)cc23)sc1C(=O)OC(C)(C)C]

Example 181.1: 7-Bromo-2-oxido-isoquinolin-2-ium (342.00 mg, 1.5264 mmol), 1-methyl-4-(4,4,5,5-tetramethyl-1,3,2-dioxoborolan-2-yl)-1 H-pyrazole (412.89 mg, 1.9844 mmol) were dissolved in dioxane (7.63 mL) and 2M aqueous Na₂CO₃ (3.7 mL) was added. The mixture was bubbled with Ar for 15 min then 1,1′-bis(di-tert-butylphosphino)ferrocenepalladium chloride (24.87 mg, 0.0382 mmol) was added and the mixture was heated at 60° C. overnight. The reaction mixture was diluted with EtOAc, filtered through celite, concentrated to dryness. Purification by silica gel column chromatography (eluent: 1-5% MeOH in DCM) to afford 7-(1-methylpyrazol-4-yl)-2-oxido-isoquinolin-2-ium (35 mg, 10%, 0.155 mmol). HPLC/MS m/z: 226.10, [M+H]⁺, Rt (P): 1.05 min.

Example 181.2: Prepared from 7-(1-methylpyrazol-4-yl)-2-oxido-isoquinolin-2-ium using procedure from Example 176 to afford tert-butyl (S)-2-(2-hydroxy-3-((7-(1-methyl-1 H-pyrazol-4-yl)isoquinolin-1-yl)amino)propanamido)-4-methylthiazole-5-carboxylate (34 mg, 43%, 0.0669 mmol). HPLC/MS m/z: 509.20, [M+H]⁺, Rt (U): 2.61 min. ¹H NMR (600 MHz, Chloroform-d) δ 10.31 (s, 1H), 9.36 (s, 1H), 7.84 (d, J=0.9 Hz, 1H), 7.82 (d, J=6.0 Hz, 1H), 7.74 (dd, J=6.7, 1.8 Hz, 2H), 7.71-7.68 (m, 2H), 7.03 (d, J=6.0 Hz, 1H), 5.91 (dd, J=6.6, 4.5 Hz, 1H), 4.61 (dd, J=4.1, 2.2 Hz, 1H), 4.19 (ddd, J=14.8, 4.6, 2.3 Hz, 1H), 4.15-4.09 (m, 1H), 3.97 (s, 3H), 2.54 (s, 3H), 1.53 (s, 9H).

### Example 182: (S)-2-hydroxy-3-((7-(5-methyl-1,2,4-oxadiazol-3-yl)isoquinolin-1-yl)amino)-N-(1-methyl-5-(5-propyl-1,2,4-oxadiazol-3-yl)-1 H-pyrazol-3-yl)propenamide

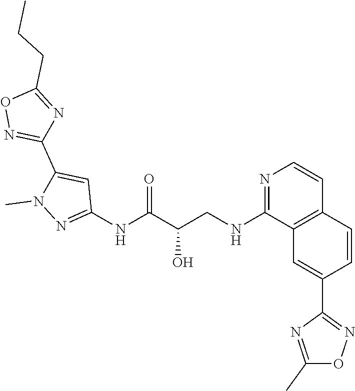 [SMILES: CCCc1nc(-c2cc(NC(=O)[C@@H](O)CNc3nccc4ccc(-c5noc(C)n5)cc34)nn2C)no1]

Example 182.1: 2-Methyl-5-nitro-pyrazole-3-carbonitrile (310.00 mg, 2.038 mmol), hydroxylamine hydrochloride (0.57 g, 8.152 mmol), and sodium hydrogen carbonate (1.37 g, 16.304 mmol) were mixed in dry ethanol (10.19 mL). MgSO₄ was added and the mixture was stirred at 40° C. overnight. The reaction mixture was concentrated in vacuo, mixed with EtOAc, washed with water, dried over anhydrous MgSO₄ and concentrated in vacuo to give N′-hydroxy-2-methyl-5-nitro-pyrazole-3-carboxamidine (380 mg, 101%, 2.0525 mmol) as a colorless powder which was used in the next reaction without further purification. HPLC/MS m/z: 186.07, [M+H]⁺, Rt (P): 0.14 min.

Example 182.2: N′-hydroxy-2-methyl-5-nitro-pyrazole-3-carboxamidine (380.00 mg, 2.0525 mmol) was dissolved in dry DMF (6.84 mL). Butyric anhydride (0.34 mL, 2.0525 mmol) was added, the reaction mixture was stirred at rt for 2.5 h. Butyric anhydride (0.17 mL, 0.5 eq) was added and the reaction mixture was stirred at 40° C. for 2 h. Water was added, the product was extracted with EtOAc, dried over MgSO₄ and concentrated in vacuo. Purification by silica gel column chromatography (eluent: 20-50% EtOAc in cyclohexane) to give N-[(Z)-N-hydroxy-C-(2-methyl-5-nitro-pyrazol-3-yl)carbonimidoyl]butanamide (497 mg, 95%, 1.9473 mmol) as a colourless oil. HPLC/MS m/z: 278.09, [M+Na]⁺, Rt (T): 1.02 min.

Example 182.3: N-[(Z)-N-hydroxy-C-(2-methyl-5-nitro-pyrazol-3-yl)carbonimidoyl]butanamide (480.00 mg, 1.8807 mmol) was dissolved in MeCN (9.40 mL). Potassium hydroxide (211.05 mg, 3.7613 mmol) was added, stirred at rt for 0.5 h. Water and EtOAc were added. The product was extracted with EtOAc, dried over MgSO₄ and concentrated in vacuo to give 3-(2-methyl-5-nitro-pyrazol-3-yl)-5-propyl-1,2,4-oxadiazole (380 mg, 85%, 1.6019 mmol) as a colorless powder. HPLC/MS m/z: 238.10, [M+H]⁺, Rt (P): 1.40 min.

Example 182.4: 3-(2-methyl-5-nitro-pyrazol-3-yl)-5-propyl-1,2,4-oxadiazole (380.00 mg, 1.6019 mmol) and iron (447.33 mg, 8.0094 mmol) were suspended in EtOH (8.01 mL). Acetic acid (0.46 mL, 8.0094 mmol) was added, and the reaction mixture was stirred at 50° C. for 1.5 h. The mixture was diluted with EtOAc and filtered. The pH was adjusted to pH˜10 with a 2 M solution of NaOH. The product was extracted with EtOAc, dried over MgSO₄ and concentrated in vacuo to give 1-methyl-5-(5-propyl-1,2,4-oxadiazol-3-yl)pyrazol-3-amine (277 mg, 83%, 1.3367 mmol) as a pale pink powder. HPLC/MS m/z: 208.14, [M+H]⁺, Rt (T): 1.15 min.

Example 182.5: 1-Methyl-5-(5-propyl-1,2,4-oxadiazol-3-yl)pyrazol-3-amine was used in a similar procedure to Example 176 to afford (S)-2-hydroxy-3-((7-(5-methyl-1,2,4-oxadiazol-3-yl)isoquinolin-1-yl)amino)-N-(1-methyl-5-(5-propyl-1,2,4-oxadiazol-3-yl)-1 H-pyrazol-3-yl)propanamide (53 mg, 57%, 0.1053 mmol) as a pale-yellow powder. HPLC/MS m/z: 504.21, [M+H]⁺, Rt (U): 2.55 min. ¹H NMR (600 MHz, DMSO-d₆) δ 10.14 (s, 1H), 8.98-8.90 (m, 1H), 8.17 (dd, J=8.5, 1.5 Hz, 1H), 8.09-8.04 (m, 1H), 7.96 (d, J=5.7 Hz, 1H), 7.87 (d, J=8.5 Hz, 1H), 7.12 (s, 1H), 7.04-6.98 (m, 1H), 6.59 (d, J=5.7 Hz, 1H), 4.51-4.44 (m, 1H), 4.04 (s, 3H), 3.96-3.88 (m, 1H), 3.74-3.66 (m, 1H), 2.99 (t, J=7.4 Hz, 2H), 2.70 (s, 3H), 1.85-1.75 (m, 2H), 0.98 (t, J=7.4 Hz, 3H).

### Example 183: (S)-2-hydroxy-N-(1-methyl-5-(5-propyl-1,2,4-oxadiazol-3-yl)-1 H-pyrazol-3-yl)-3-((7-methylisoquinolin-1-yl)amino)propanamide

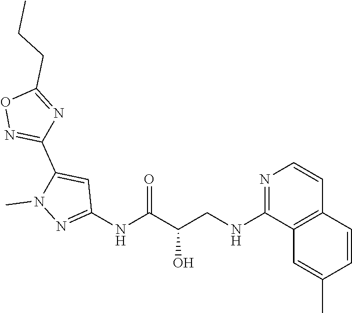 [SMILES: CCCc1nc(-c2cc(NC(=O)[C@@H](O)CNc3nccc4ccc(C)cc34)nn2C)no1]

Example 183.1: A mixture of 1-chloro-7-methylisoquinoline (2.00 g, 10.696 mmol) in dry THF (112.59 mL) was degassed by bubbling nitrogen for 5 min. Then, [1,1′-Bis(diphenylphosphino)ferrocene]dichloropalladium(II), complex with dichloromethane (0.44 g, 0.5348 mmol), N,N,N′,N′-Tetramethylethylenediamine (2.73 mL, 18.184 mmol) and sodium borohydride (0.69 g, 18.184 mmol) were added. The reaction was stirred at rt under argon for 3 h. The reaction mixture was filtered, concentrated in vacuo, mixed with saturated NaCl (100 mL) and extracted with EtOAc (3×50 mL). The combined organic phase was washed with saturated NaCl (3×20 mL), dried over anhydrous MgSO₄ and concentrated in vacuo. Purification by silica gel column chromatography (eluent: 0-20% EtOAc in cyclohexane) afforded 7-methylisoquinoline (1.1 g, 72%, 7.6821 mmol) as an off-white solid. HPLC/MS m/z: 144.08, [M+H]⁺, Rt (T): 0.6 min.

Example 183.2: 7-methylisoquinoline (1.00 g, 6.6345 mmol) was dissolved in CHCl₃ (11.64 mL) and cooled in an ice bath. m-CPBA (1.78 g, 7.9614 mmol) was added. The reaction was allowed to warm to ambient temperature. Another 1 g of m-CPBA was added to the reaction after 4 h. The reaction was continued to stir overnight. K₂CO₃ (3.67 g, 26.538 mmol) was added, and the mixture was stirred for 1 h before filtering through a pad of anhydrous MgSO₄. The filtrate was concentrated in vacuo to yield 7-methyl-2-oxido-isoquinolin-2-ium (0.91 g, 86%, 5.7168 mmol) as an amorphous, pale purple solid. HPLC/MS m/z: 160.08, [M+H]⁺, Rt (T): 0.86 min.

Example 183.3: Prepared from 1-methyl-5-(5-propyl-1,2,4-oxadiazol-3-yl)pyrazol-3-amine [Example 182] and 7-methyl-2-oxido-isoquinolin-2-ium to afford (S)-2-hydroxy-N-(1-methyl-5-(5-propyl-1,2,4-oxadiazol-3-yl)-1 H-pyrazol-3-yl)-3-((7-methylisoquinolin-1-yl)amino)propanamide (48 mg, 56%, 0.1102 mmol). HPLC/MS m/z: 436.21, [M+H]⁺, Rt (U): 2.44 min. ¹H NMR (600 MHz, DMSO-d₆) δ 10.11 (s, 1H), 8.08-8.03 (m, 1H), 7.80 (d, J=5.8 Hz, 1H), 7.64 (d, J=8.2 Hz, 1H), 7.54 (t, J=5.5 Hz, 1H), 7.49 (dd, J=8.4, 1.6 Hz, 1H), 7.12 (s, 1H), 7.01 (s, 1H), 6.92 (d, J=5.9 Hz, 1H), 4.45-4.38 (m, 1H), 4.04 (s, 3H), 3.93-3.85 (m, 1H), 3.74-3.65 (m, 1H), 3.00 (t, J=7.4 Hz, 2H), 2.46 (s, 3H), 1.81 (sext, J=7.4 Hz, 2H), 0.99 (t, J=7.4 Hz, 3H).

### Example 184: tert-Butyl (R)-2-(2-hydroxy-3-((7-methylisoquinolin-1-yl)amino)propanamido)-4-methylthiazole-5-carboxylate

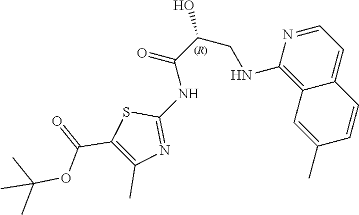

Example 184.1: To anhydrous methanol (11.32 mL) at −10° C. under nitrogen, was carefully added thionyl chloride (1.18 mL, 16.153 mmol). After stirring for 5 min (R)-3-amino-2-hydroxypropanoic acid (0.68 g, 6.4611 mmol) was added in three portions. The reaction mixture was stirred at −10° C. for 5 min before being allowed to warm to rt and stirred overnight. The solvent was removed in vacuo using the addition of toluene to aid the removal of all the volatiles to afford [(2R)-2-hydroxy-3-methoxy-3-oxo-propyl]ammonium chloride (988 mg, 98%, 6.3504 mmol) as an off-white powder. ¹H NMR (600 MHz, DMSO-d₆) δ 8.10 (s, 3H), 6.34 (d, J=5.6 Hz, 1H), 4.37 (dt, J=8.8, 4.6 Hz, 1H), 3.68 (s, 3H), 3.10 (dd, J=13.0, 3.8 Hz, 1H), 2.91 (dd, J=13.0, 8.6 Hz, 1H).

Example 184.2: 7-Methylisoquinoline 2-oxide [Example 183](200.00 mg, 1.2564 mmol) and methyl (2R)-3-amino-2-hydroxy-propanoate hydrochloride (293.22 mg, 1.8847 mmol) were dissolved/suspended in DCM (4.19 mL). DIPEA (1.04 mL, 5.9681 mmol) was added followed by PyBrop (761.44 mg, 1.6334 mmol) and stirred for 56 h. Purified by silica gel column chromatography (eluent: 0-20% (EtOAc/MeOH 3/1) in cyclohexane). Further purification by reverse phase column chromatography (eluent: 5-40% MeOH in water (+0.1% formic acid)) afforded methyl (R)-2-hydroxy-3-((7-methylisoquinolin-1-yl)amino)propanoate formate (107 mg, 28%, 0.3493 mmol) as a colorless solid. HPLC/MS m/z: 261.1 [M+H]⁺, Rt (U): 1.35 min.

Example 184.3: Methyl (R)-2-hydroxy-3-((7-methylisoquinolin-1-yl)amino)propanoate formate (105.00 mg, 0.3428 mmol) was dissolved in THE (1.71 mL). Water (1.71 mL) was added followed by LiOH (43.15 mg, 1.0284 mmol). The resulting solution was stirred at rt for 3.5 h. Additional LiOH (14 mg, 1 eq.) was added and stirred at rt overnight. Additional LiOH (14 mg, 1 eq.) was added and stirred for further 25 h overnight. Acidified with HCl pH=3 before the volatiles were removed in vacuo. Purified by reverse phase column chromatography (eluent: 5-70% MeOH in water (+0.1% formic acid modifier in both)) to afford (2R)-2-hydroxy-3-[(7-methyl-1-isoquinolyl)amino]propanoic acid (73 mg, 86%, 0.2964 mmol) as a clear solid. HPLC/MS m/z: 247.1 [M+H]⁺, Rt (T): 0.75 min.

Example 184.4: (2R)-2-hydroxy-3-[(7-methyl-1-isoquinolyl)amino]propanoic acid (33.00 mg, 0.1340 mmol) was suspended in dry DCM (1.34 mL) in a microwave vial. 1-Hydroxybenzotriazole (45.27 mg, 0.3350 mmol) was added followed by 3-(ethyliminomethyleneamino)-N,N-dimethyl-propan-1-amine hydrochloride (64.22 mg, 0.3350 mmol). Stirred for 10 min at rt before the addition of tert-butyl 2-amino-4-methyl-thiazole-5-carboxylate [Example 110.1](58.00 mg, 0.2707 mmol). Stirred at rt overnight. Purified by reverse phase column chromatography (eluent: 20-100% MeOH in water (+0.1% formic acid modifier in both)) followed by amino silica gel column chromatography (Biotage KP-NH) (eluent: 1:1 EtOAc/cyclohexane) to afford tert-butyl (R)-2-(2-hydroxy-3-((7-methylisoquinolin-1-yl)amino)propanamido)-4-methylthiazole-5-carboxylate (6 mg, 10%, 0.0136 mmol). HPLC/MS m/z: 443.2 [M+H]⁺, Rt (U): 2.63 min. ¹H NMR (600 MHz, DMSO-d₆) δ 8.02 (s, 1H), 7.76 (d, J=5.8 Hz, 1H), 7.63 (d, J=8.2 Hz, 1H), 7.52 (t, J=5.6 Hz, 1H), 7.48 (dd, J=8.3, 1.6 Hz, 1H), 6.91 (d, J=5.8 Hz, 1H), 4.54 (t, J=5.2 Hz, 1H), 3.83 (dt, J=13.8, 5.0 Hz, 1H), 3.77 (dt, J=13.7, 5.9 Hz, 1H), 2.50 (s, 3H), 2.45 (s, 3H), 1.50 (s, 9H). (Note: 1×NH, 1×OH not observed)

The following examples were prepared by an analogous procedure:

### Example 185: tert-Butyl (S)-2-(2-hydroxy-3-((7-methylisoquinolin-1-yl)amino)propanamido)-4-methylthiazole-5-carboxylate

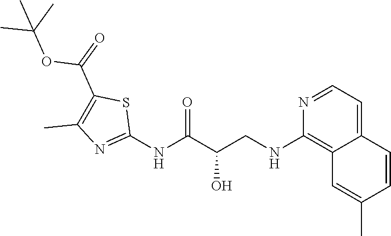 [SMILES: Cc1ccc2ccnc(NC[C@H](O)C(=O)Nc3nc(C)c(C(=O)OC(C)(C)C)s3)c2c1]

Prepared using (S)-methyl-3-amino-2-hydroxypropanoate hydrochloride to afford tert-butyl (S)-2-(2-hydroxy-3-((7-methylisoquinolin-1-yl)amino)propanamido)-4-methylthiazole-5-carboxylate (6 mg, 13%, 0.0136 mmol) as a colorless solid. HPLC/MS m/z: 443.18, [M+H]⁺, Rt (U): 2.60 min. ¹H NMR (600 MHz, DMSO-d₆) δ 8.04 (s, 1H), 7.77 (d, J=5.9 Hz, 1H), 7.65 (d, J=8.2 Hz, 1H), 7.50 (d, J=8.2 Hz, 1H), 6.93 (d, J=5.9 Hz, 1H), 4.58-4.53 (m, 1H), 3.85 (dt, J=13.8, 5.0 Hz, 1H), 3.79 (dt, J=13.8, 5.9 Hz, 1H), 2.52-2.50 (m, 3H), 2.46 (s, 3H), 1.51 (s, 9H). (Note: 2×NH, 1×OH not observed).

### Example 186: Propyl (S)-2-(2-methoxy-3-((7-(5-methyl-1,2,4-oxadiazol-3-yl)isoquinolin-1-yl)amino)propanamido)-4-methylthiazole-5-carboxylate

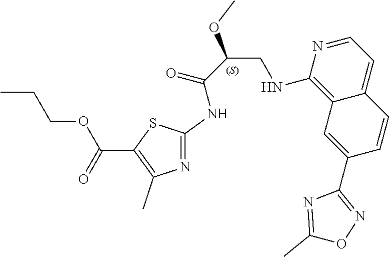

Prepared using 5-methyl-3-(2-oxidoisoquinolin-2-ium-7-yl)-1,2,4-oxadiazole [Example 96] and methyl (2S)-3-amino-2-methoxyproponate hydrochloride in an analogous procedure to Example 184 to afford propyl (S)-2-(2-methoxy-3-((7-(5-methyl-1,2,4-oxadiazol-3-yl)isoquinolin-1-yl)amino)propanamido)-4-methylthiazole-5-carboxylate (5 mg, 23%, 0.0098 mmol) as an off-white solid. HPLC/MS m/z: 511.2 [M+H]⁺, Rt (U): 2.73 min. ¹H NMR (600 MHz, DMSO-d₆) δ 12.73 (s, 1H), 8.89 (s, 1H), 8.15 (dd, J=8.5, 1.5 Hz, 1H), 8.09 (t, J=5.7 Hz, 1H), 7.91 (d, J=5.7 Hz, 1H), 7.85 (d, J=8.5 Hz, 1H), 6.97 (d, J=5.7 Hz, 1H), 4.35 (t, J=6.0 Hz, 1H), 4.16 (t, J=6.5 Hz, 2H), 3.89 (dt, J=13.5, 5.7 Hz, 1H), 3.81 (dt, J=13.5, 5.8 Hz, 1H), 3.33 (s, 3H), 2.69 (s, 3H), 2.53 (s, 3H), 1.67 (sext, J=7.1 Hz, 2H), 0.94 (t, J=7.4 Hz, 3H).

### Example 187: (S)-N-(5-(tert-butyl)-4-methylthiazol-2-yl)-2-hydroxy-3-((7-(5-methyl-1,2,4-oxadiazol-3-yl)isoquinolin-1-yl)amino)propenamide

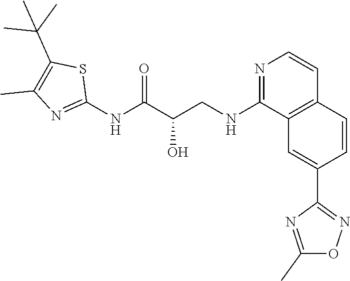 [SMILES: Cc1nc(-c2ccc3ccnc(NC[C@H](O)C(=O)Nc4nc(C)c(C(C)(C)C)s4)c3c2)no1]

Prepared using 5-methyl-3-(2-oxidoisoquinolin-2-ium-7-yl)-1,2,4-oxadiazole [Example 96], methyl (2S)-3-amino-2-hydroxy-propanoate hydrochloride and 5-tert-butyl-4-methyl-thiazol-2-ylamine to afford (S)-N-(5-(tert-butyl)-4-methylthiazol-2-yl)-2-hydroxy-3-((7-(5-methyl-1,2,4-oxadiazol-3-yl)isoquinolin-1-yl)amino)propanamide (36 mg, 49%, 0.0772 mmol). HPLC/MS m/z: 467.19, [M+H]⁺, Rt (Q): 2.67 min. ¹H NMR (600 MHz, Chloroform-d) δ 8.51-8.48 (m, 1H), 8.24 (dd, J=8.5, 1.5 Hz, 1H), 7.93 (d, J=6.0 Hz, 1H), 7.76 (d, J=8.5 Hz, 1H), 7.06-7.01 (m, 1H), 6.27 (d, J=5.8 Hz, 1H), 4.61 (dd, J=4.4, 2.3 Hz, 1H), 4.23-4.10 (m, 2H), 2.66 (s, 3H), 2.29 (s, 3H), 1.33 (s, 9H). (Note: 1×NH, 1×OH not observed).

### Example 188: (S)-2-hydroxy-3-((7-(5-methyl-1,2,4-oxadiazol-3-yl)isoquinolin-1-yl)amino)-N-(1-methyl-5-pentyl-1 H-pyrazol-3-yl)propenamide

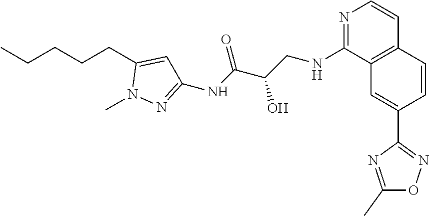 [SMILES: CCCCCc1cc(NC(=O)[C@@H](O)CNc2nccc3ccc(-c4noc(C)n4)cc23)nn1C]

Prepared using 5-methyl-3-(2-oxidoisoquinolin-2-ium-7-yl)-1,2,4-oxadiazole [Example 96], methyl (2S)-3-amino-2-hydroxy-propanoate hydrochloride and 1-methyl-5-pentyl-pyrazol-3-amine to afford (S)-2-hydroxy-3-((7-(5-methyl-1,2,4-oxadiazol-3-yl)isoquinolin-1-yl)amino)-N-(1-methyl-5-pentyl-1 H-pyrazol-3-yl)propanamide (9 mg, 22%, 0.0186 mmol). HPLC/MS m/z: 464.24, [M+H]⁺, Rt (U): 2.69 min. ¹H NMR (600 MHz, Chloroform-d) δ 9.40 (s, 1H), 8.52-8.48 (m, 1H), 8.24 (dd, J=8.4, 1.5 Hz, 1H), 7.90 (d, J=6.0 Hz, 1H), 7.74 (d, J=8.5 Hz, 1H), 7.01 (d, J=6.0 Hz, 1H), 6.48 (s, 1H), 6.36 (s, 1H), 4.52 (dd, J=4.5, 2.3 Hz, 1H), 4.23-4.06 (m, 2H), 3.59 (s, 3H), 2.66 (s, 3H), 2.47 (td, J=7.4, 1.8 Hz, 2H), 1.63-1.51 (m, 2H), 1.40-1.26 (m, 4H), 0.94-0.83 (m, 3H). (Note: 1×OH not observed).

### Example 189: (R)-2-hydroxy-3-((7-(5-methyl-1,2,4-oxadiazol-3-yl)isoquinolin-1-yl)amino)-N-(1-methyl-5-pentyl-1 H-pyrazol-3-yl)propenamide

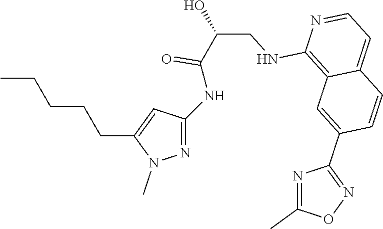 [SMILES: CCCCCc1cc(NC(=O)[C@H](O)CNc2nccc3ccc(-c4noc(C)n4)cc23)nn1C]

Prepared using 5-methyl-3-(2-oxidoisoquinolin-2-ium-7-yl)-1,2,4-oxadiazole [Example 96] and 1-methyl-5-pentyl-pyrazol-3-amine to afford (R)-2-hydroxy-3-((7-(5-methyl-1,2,4-oxadiazol-3-yl)isoquinolin-1-yl)amino)-N-(1-methyl-5-pentyl-1 H-pyrazol-3-yl)propanamide (5.7 mg, 20%, 0.0123 mmol). HPLC/MS m/z: 464.24 [M+H]⁺, Rt (U): 2.62 min. ¹H NMR (600 MHz, Chloroform-d) δ 9.34 (s, 1H), 8.50 (s, 1H), 8.25 (dd, J=8.4, 1.5 Hz, 1H), 7.91 (d, J=5.9 Hz, 1H), 7.76 (d, J=8.5 Hz, 1H), 7.02 (d, J=6.0 Hz, 1H), 6.49 (s, 1H), 6.22 (t, J=5.7 Hz, 1H), 4.50 (dd, J=4.5, 2.3 Hz, 1H), 4.14 (dddd, J=16.3, 14.7, 11.3, 2.2 Hz, 2H), 3.60 (s, 3H), 2.67 (s, 3H), 2.48 (td, J=7.4, 1.7 Hz, 2H), 1.59 (sext, J=7.5 Hz, 2H), 1.32 (dt, J=7.3, 3.8 Hz, 4H), 0.92-0.85 (m, 3H). (Note: 1×OH not observed).

### Example 190: (R)-N-(5-(tert-butyl)-4-methylthiazol-2-yl)-2-hydroxy-3-((7-(5-methyl-1,2,4-oxadiazol-3-yl)isoquinolin-1-yl)amino)propanamide

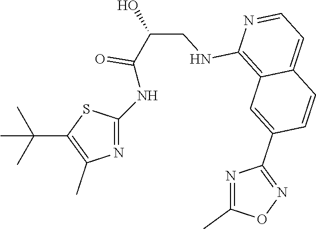 [SMILES: Cc1nc(-c2ccc3ccnc(NC[C@@H](O)C(=O)Nc4nc(C)c(C(C)(C)C)s4)c3c2)no1]

Prepared using 5-methyl-3-(2-oxidoisoquinolin-2-ium-7-yl)-1,2,4-oxadiazole [Example 96] and 5-tert-butyl-4-methyl-thiazol-2-ylamine to afford (R)-N-(5-(tert-butyl)-4-methylthiazol-2-yl)-2-hydroxy-3-((7-(5-methyl-1,2,4-oxadiazol-3-yl)isoquinolin-1-yl)amino)propanamide (2.8 mg, 12%, 0.0058 mmol). HPLC/MS m/z: 467.19, [M+H]⁺, Rt (U): 2.74 min. ¹H NMR (600 MHz, Chloroform-d) δ 8.49-8.47 (m, 1H), 8.26 (dd, J=8.5, 1.5 Hz, 1H), 7.92 (d, J=6.0 Hz, 1H), 7.77 (d, J=8.5 Hz, 1H), 7.06-7.01 (m, 1H), 6.16 (t, J=5.7 Hz, 1H), 4.59 (dd, J=4.4, 2.2 Hz, 1H), 4.18 (ddd, J=14.8, 4.8, 2.2 Hz, 1H), 4.12 (ddd, J=14.9, 6.4, 4.5 Hz, 1H), 2.67 (s, 3H), 2.30 (s, 3H), 1.36 (s, 9H). (Note: 1×NH, 1×OH not observed).

### Example 191: (R)-2-hydroxy-N-(5-isobutyl-4-methylthiazol-2-yl)-3-((7-(5-methyl-1,2,4-oxadiazol-3-yl)isoquinolin-1-yl)amino)propenamide

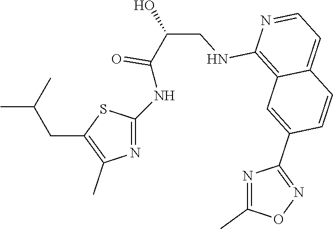 [SMILES: Cc1nc(-c2ccc3ccnc(NC[C@@H](O)C(=O)Nc4nc(C)c(CC(C)C)s4)c3c2)no1]

Prepared using 5-methyl-3-(2-oxidoisoquinolin-2-ium-7-yl)-1,2,4-oxadiazole [Example 96] and 4-methyl-5-(2-methylpropyl)-1,3-thiazol-2-amine hydrochloride to afford (R)-2-hydroxy-N-(5-isobutyl-4-methylthiazol-2-yl)-3-((7-(5-methyl-1,2,4-oxadiazol-3-yl)isoquinolin-1-yl)amino)propanamide (19 mg, 43%, 0.0407 mmol). HPLC/MS m/z: 467.18, [M+H]⁺, Rt (Q): 2.68 min. ¹H NMR (600 MHz, Chloroform-d) δ 8.49 (s, 1H), 8.26 (dd, J=8.5, 1.5 Hz, 1H), 7.93 (d, J=6.0 Hz, 1H), 7.77 (d, J=8.4 Hz, 1H), 7.05 (d, J=6.0 Hz, 1H), 6.21 (s, 1H), 4.61 (dd, J=4.6, 2.3 Hz, 1H), 4.22-4.10 (m, 2H), 2.68 (s, 3H), 2.48 (dd, J=7.1, 1.4 Hz, 2H), 2.15 (s, 3H), 1.84-1.74 (m, 1H), 0.90 (dd, J=7.9, 6.6 Hz, 6H). (Note: 1×NH not observed).

### Example 192: (R)-2-hydroxy-3-((7-(5-methyl-1,2,4-oxadiazol-3-yl)isoquinolin-1-yl)amino)-N-(4-methyl-5-(1-(trifluoromethyl)cyclopropyl)thiazol-2-yl)propanamide

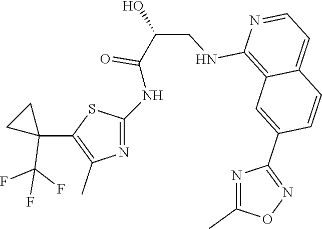 [SMILES: Cc1nc(-c2ccc3ccnc(NC[C@@H](O)C(=O)Nc4nc(C)c(C5(C(F)(F)F)CC5)s4)c3c2)no1]

Prepared using 5-methyl-3-(2-oxidoisoquinolin-2-ium-7-yl)-1,2,4-oxadiazole [Example 96] and 4-methyl-5-[1-(trifluoromethyl)cyclopropyl]thiazol-2-amine [Example 51.1] to afford (R)-2-hydroxy-3-((7-(5-methyl-1,2,4-oxadiazol-3-yl)isoquinolin-1-yl)amino)-N-(4-methyl-5-(1-(trifluoromethyl)cyclopropyl)thiazol-2-yl)propanamide (5 mg, 10%, 0.0096 mmol). HPLC/MS m/z: 519.14, [M+H]⁺, Rt (U): 2.71 min. ¹H NMR (600 MHz, Chloroform-d) δ 10.22-10.08 (m, 1H), 9.04 (s, 1H), 8.49-8.45 (m, 1H), 8.30-8.25 (m, 1H), 7.92 (d, J=5.9 Hz, 1H), 7.79 (d, J=8.5 Hz, 1H), 7.09-7.06 (m, 1H), 6.06 (t, J=5.7 Hz, 1H), 4.59 (dd, J=4.4, 2.2 Hz, 1H), 4.18 (ddd, J=14.8, 4.8, 2.3 Hz, 1H), 4.12 (ddd, J=14.8, 6.4, 4.3 Hz, 1H), 2.69 (d, J=4.2 Hz, 3H), 2.28 (s, 3H), 1.45-1.40 (m, 2H), 1.05 (s, 2H).

### Example 193: tert-Butyl 2-[(3S)-4-cyano-3-{[3-(5-methyl-1,2,4-oxadiazol-3-yl)phenyl]form, amido}-butanamido]-4-methyl-1,3-thiazole-5-carboxylate

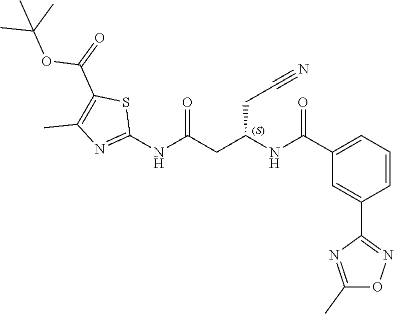

### Example 193.1: To a stirred solution of 3-(5-methyl-1,2,4-oxadiazol-3-yl)benzoic acid (15.00 g, 73.464 mmol) and 1,5-dimethyl 3-aminopentanedioate hydrochloride (15.55 g, 73.473 mmol) in DMF (150.0 mL) were added TCFH (30.92 g, 110.200 mmol) and 1-methyl-1 H-imidazole (18.1 g, 220.452 mmol), and the resulting mixture was stirred at rt for 16 h. The resulting mixture was diluted with water (150 mL) and extracted with ethyl acetate. The combined organic layers were washed with brine, dried over anhydrous sodium sulfate, filtered, and concentrated under reduced pressure. The residue was purified by silica gel column chromatography (eluent: ethyl acetate/petroleum ether—2:1) to afford 20.00 g (75%) of 1,5-dimethyl 3-{[3-(5-methyl-1,2,4-oxadiazol-3-yl)phenyl]formamido}pentanedioate as a yellow solid. HPLC/MS m/z: 362.20 [M+H]⁺, Rt (I): 0.86 min.

Example 193.2: To a solution of 1,5-dimethyl 3-{[3-(5-methyl-1,2,4-oxadiazol-3-yl)phenyl]-formamido}pentanedioate (10.00 g, 27.674 mmol) in THF (80.0 mL) and water (40.0 mL) was added lithium hydroxide (662.8 mg, 27.674 mmol), and the resulting mixture was stirred at rt for 16 h. The mixture was acidified to pH5 with 2N aqueous HCl solution, and the resulting mixture was extracted with dichloromethane/methanol—10:1. The combined organic layers were washed with brine, dried over anhydrous sodium sulfate, filtered, and concentrated under reduced pressure. The residue was purified by silica gel column chromatography (eluent: dichloromethane/methanol—10:1) to afford 4.00 g (42%) of 5-methoxy-3-{[3-(5-methyl-1,2,4-oxadiazol-3-yl)phenyl], formamido}-5-oxopentanoic acid as a yellow solid. HPLC/MS m/z: 348.05 [M+H]⁺, Rt (C): 0.67 min.

Example 193.3: 5-methoxy-3-{[3-(5-methyl-1,2,4-oxadiazol-3-yl)phenyl]formamido}-5-oxopentanoic acid (4.00 g, 11.517 mmol) and ammonium chloride (924 mg, 17.274 mmol) were dissolved in DMF (40.0 mL). Diethylamine (4.46 g, 34.520 mmol) and HATU (6.57 g, 17.279 mmol) were added, and the resulting mixture was stirred at rt for 16 h. The resulting mixture was diluted with water (40 mL) and extracted with dichloromethane/methanol—10:1. The combined organic layers were washed with brine, dried over anhydrous sodium sulfate, filtered, and concentrated under reduced pressure. The residue was purified by silica gel column chromatography (eluent: dichloromethane/methanol—10:1) to give 2.80 g (69%) of methyl 4-carbamoyl-3-{[3-(5-methyl-1,2,4-oxadiazol-3-yl)phenyl]-formamido}butanoate as a colorless solid. HPLC/MS m/z: 347.20 [M+H]⁺, Rt (I): 0.69 min.

Example 193.4: A solution of methyl 4-carbamoyl-3-{[3-(5-methyl-1,2,4-oxadiazol-3-yl)phenyl]-formamido}butanoate (800.0 mg, 2.31 mmol) in EtOH (10.0 mL) was treated with a solution of lithium hydroxide (276.6 mg, 11.549 mmol) in water (4.0 mL). The resulting mixture was stirred at rt for 1 h. The mixture was acidified to pH5 with 2N aqueous HCl solution. The aqueous layer was extracted with dichloromethane. The combined organic layers were dried over anhydrous sodium sulfate, filtered, and concentrated under reduced pressure to afford 750 mg (98%) 4-carbamoyl-3-{[3-(5-methyl-1,2,4-oxadiazol-3-yl)phenyl]formamidolbutanoic acid as a yellow solid. HPLC/MS m/z: 333.10 [M+H]⁺, Rt (C): 0.54 min.

Example 193.5: A stirred solution of 4-carbamoyl-3-{[3-(5-methyl-1,2,4-oxadiazol-3-yl)phenyl]-formamido}butanoic acid (300.0 mg, 0.903 mmol) in DMF (5.0 mL) was cooled to 0 0° C. POCI₃ (277.0 mg, 1.807 mmol) was added dropwise at 0° C. After complete addition the reaction mixture was warmed to rt and stirred for 4 h. The reaction mixture was quenched by the addition of water (5 mL) at rt. The resulting mixture was extracted with ethyl acetate, the combined organic layers were washed with brine, dried over anhydrous sodium sulfate, filtered, and concentrated under reduced pressure. The residue was purified by silica gel column chromatography (eluent: ethyl acetate/petroleum ether—1:1) to afford 240 mg (85%) of 4-cyano-3-{[3-(5-methyl-1,2,4-oxadiazol-3-yl)phenyl]formamido}butanoic acid as a yellow solid. HPLC/MS m/z: 315.05 [M+H]⁺, Rt (C): 0.62 min.

Example 193.6: A stirred solution of 4-cyano-3-{[3-(5-methyl-1,2,4-oxadiazol-3-yl)phenyl]form-amido}butanoic acid (220.0 mg, 0.700 mmol) and tert-butyl 2-amino-4-methyl-1,3-thiazole-5-carboxylate (150.0 mg, 0.700 mmol) in DMF (3.0 mL) was treated with [chloro(dimethylamino)methylidene]dimethylazanium; hexafluoro-λ⁵-phosphanuide (294.6 mg, 1.050 mmol) and 1-methyl-1 H-imidazole (172.4 mg, 2.100 mmol) and the resulting mixture was stirred at rt for 2 h. The resulting mixture was concentrated under vacuum, and the residue was purified by RP flash chromatography to give 150 mg (42.0%) of tert-butyl 2-(4-cyano-3-{[3-(5-methyl-1,2,4-oxadiazol-3-yl)phenyl]formamido}butanamido)-4-methyl-1,3-thiazole-5-carboxylate as a colorless solid (HPLC/MS m/z: 511.15 [M+H]⁺, Rt (C): 0.95 min), which was separated into the enantiomers by preparative chiral HPLC (column: CHIRALPAK IF-3, 4.6×50 mm, 3 μm, eluent: tert-butyl methyl ether (containing 0.1% diethylamine)/EtOH—93:7). Yield: 32 mg (21%) of tert-butyl 2-[(3S)-4-cyano-3-{[3-(5-methyl-1,2,4-oxadiazol-3-yl)phenyl]formamido}butanamido]-4-methyl-1,3-thiazole-5-carboxylate as a colorless solid. HPLC/MS m/z: 511.15 [M+H]⁺, Rt (E): 0.90 min. ¹H NMR (300 MHz, DMSO-d₆): δ 12.54 (s, 1H), 8.96 (d, J=7.8 Hz, 1H), 8.45 (t, J=1.8 Hz, 1H), 8.15 (dt, J=7.7, 1.4 Hz, 1H), 8.02 (dt, J=7.9, 1.4 Hz, 1H), 7.67 (t, J=7.8 Hz, 1H), 4.68 (q, J=6.5 Hz, 1H), 3.03-2.80 (m, 4H), 2.67 (s, 3H), 2.51-2.49 (m, 3H), 1.47 (s, 9H). Chiral HPLC: Rt: 4.25 min.

### Example 194: tert-Butyl 2-[(3R)-4-cyano-3-{[3-(5-methyl-1,2,4-oxadiazol-3-yl)phenyl]form-amido}butanamido]-4-methyl-1,3-thiazole-5-carboxylate

36 mg (24%) as a colorless solid. HPLC/MS m/z: 511.15 [M+H]⁺, Rt (E): 0.89 min. ¹H NMR (300 MHz, DMSO-d₆): δ 12.54 (s, 1H), 8.96 (d, J=7.8 Hz, 1H), 8.45 (t, J=1.7 Hz, 1H), 8.15 (dt, J=7.7, 1.4 Hz, 1H), 8.02 (dt, J=7.9, 1.4 Hz, 1H), 7.67 (t, J=7.8 Hz, 1H), 4.72-4.61 (m, 1H), 3.03-2.80 (m, 4H), 2.67 (s, 3H), 2.52-2.49 (m, 3H), 1.47 (s, 9H). Chiral HPLC: Rt: 3.20 min.

The following examples were prepared analogously:

### Example 195: Propan-2-yl 2-[(3S)-4-cyano-3-{[3-(5-methyl-1,2,4-oxadiazol-3-yl)phenyl]form-amido}butanamido]-4-methyl-1,3-thiazole-5-carboxylate

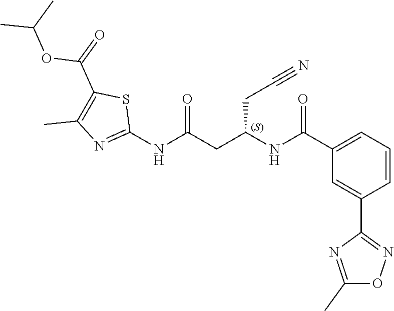

114 mg (8%) as a colorless solid. HPLC/MS m/z: 497.15 [M+H]⁺, Rt (C): 1.01 min. ¹H NMR (300 MHz, DMSO-d₆): δ 12.60 (s, 1H), 8.98 (d, J=7.8 Hz, 1H), 8.47 (d, J=2.1 Hz, 1H), 8.17 (d, J=7.7 Hz, 1H), 8.04 (d, J=7.8 Hz, 1H), 7.69 (t, J=7.8 Hz, 1H), 5.11-4.97 (m, 1H), 4.77-4.64 (m, 1H), 3.05-2.82 (m, 4H), 2.70 (s, 3H), 2.53 (s, 3H), 1.27 (d, J=6.2 Hz, 6H). Chiral HPLC: Rt: 1.61 min.

### Example 196: Propan-2-yl 2-[(3R)-4-cyano-3-{[3-(5-methyl-1,2,4-oxadiazol-3-yl)phenyl]form-amido}butanamido]-4-methyl-1,3-thiazole-5-carboxylate

120 mg (8%) as a pale-yellow solid. HPLC/MS m/z: 497.15 [M+H]⁺, Rt (C): 1.01 min. ¹H NMR (300 MHz, DMSO-d₆): δ 12.60 (s, 1H), 8.98 (d, J=7.8 Hz, 1H), 8.47 (d, J=1.8 Hz, 1H), 8.21-8.12 (m, 1H), 8.08-7.99 (m, 1H), 7.69 (t, J=7.8 Hz, 1H), 5.11-4.97 (m, 1H), 4.77-4.64 (m, 1H), 3.05-2.83 (m, 4H), 2.69 (s, 3H), 2.53 (s, 3H), 1.27 (d, J=6.2 Hz, 6H). Chiral HPLC: Rt: 0.98 min.

### Example 197: Methyl 2-[(3S)-4-cyano-3-{[3-(5-methyl-1,2,4-oxadiazol-3-yl)phenyl]formamido}, butanamido]-4-methyl-1,3-thiazole-5-carboxylate

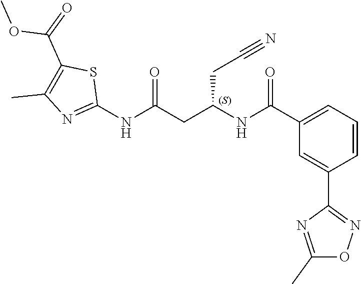

40.5 mg (24%) as a colorless solid. HPLC/MS m/z: 469.10 [M+H]⁺, Rt (J): 1.21 min. ¹H NMR (300 MHz, DMSO-d₆): δ 12.56 (s, 1H), 8.96 (d, J=7.8 Hz, 1H), 8.45 (t, J=1.7 Hz, 1H), 8.14 (dt, J=7.8, 1.4 Hz, 1H), 8.02 (dt, J=8.0, 1.4 Hz, 1H), 7.67 (t, J=7.8 Hz, 1H), 4.68 (q, J=6.5 Hz, 1H), 3.74 (s, 3H), 3.01-2.82 (m, 4H), 2.67 (s, 3H), 2.52 (s, 3H).

### Example 198: Methyl 2-[(3R)-4-cyano-3-{[3-(5-methyl-1,2,4-oxadiazol-3-yl)phenyl]formamidolbutanamido]-4-methyl-1,3-thiazole-5-carboxylate

2 mg (7%) as a colorless solid. HPLC/MS m/z: 469.00 [M+H]⁺, Rt (K): 1.10 min. ¹H NMR (400 MHz, DMSO-d₆): δ 12.46 (s, 1H), 9.00 (d, J=7.8 Hz, 1H), 8.47 (t, J=1.7 Hz, 1H), 8.16 (dt, J=7.7, 1.5 Hz, 1H), 8.04 (dt, J=7.8, 1.5 Hz, 1H), 7.69 (t, J=7.8 Hz, 1H), 4.75-4.65 (m, 1H), 3.76 (s, 3H), 3.04-2.84 (m, 4H), 2.70 (s, 3H), 2.53 (s, 3H).

### Example 199: Propan-2-yl 2-[(3S)-4-cyano-N-methyl-3-{[3-(5-methyl-1,2,4-oxadiazol-3-yl)phenyl]-formamido}butanamido]-4-methyl-1,3-thiazole-5-carboxylate

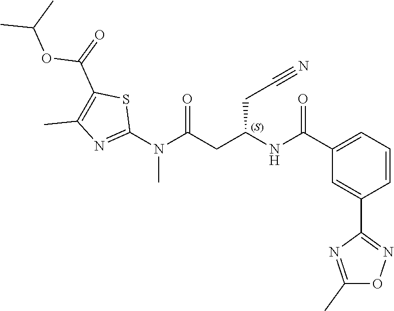

5.5 mg (10%) as a colorless solid. HPLC/MS m/z: 511.10 [M+H]⁺, Rt (C): 0.95 min. ¹H NMR (300 MHz, DMSO-d₆): δ 8.93 (d, J=7.4 Hz, 1H), 8.45 (d, J=1.8 Hz, 1H), 8.20-8.11 (m, 1H), 8.07-7.98 (m, 1H), 7.68 (t, J=7.8 Hz, 1H), 5.03 (sept, J=6.2 Hz, 1H), 4.70 (d, J=6.9 Hz, 1H), 3.66 (s, 3H), 3.27-2.88 (m, 4H), 2.61 (d, J=38.0 Hz, 6H), 1.25 (dd, J=6.3, 1.3 Hz, 6H).

### Example 200: Propan-2-yl 2-[(3R)-4-cyano-N-methyl-3-{[3-(5-methyl-1,2,4-oxadiazol-3-yl)phenyl]-formamido}butanamido]-4-methyl-1,3-thiazole-5-carboxylate

3.8 mg (6%) as a colorless solid. HPLC/MS m/z: 511.10 [M+H]⁺, Rt (C): 0.96 min. ¹H NMR (300 MHz, DMSO-d₆): δ 8.45 (t, J=1.7 Hz, 1H), 8.15 (dt, J=7.8, 1.3 Hz, 1H), 8.07-7.98 (m, 1H), 7.68 (t, J=7.8 Hz, 1H), 5.03 (sept, J=6.2 Hz, 1H), 4.74-4.63 (m, 1H), 3.66 (s, 3H), 3.21-2.89 (m, 4H), 2.67 (s, 3H), 2.55 (s, 3H), 1.25 (dd, J=6.2, 1.3 Hz, 6H).

### Example 201: tert-Butyl 2-[(3S)-4-cyano-3-{[3-(1-methyl-1 H-pyrazol-4-yl)phenyl]formamidolbutanamido]-4-methyl-1,3-thiazole-5-carboxylate

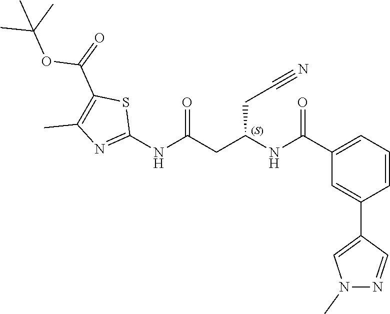

Separation of the enantiomers was performed by preparative chiral HPLC (column: Chiral Cellulose-SB, 4.6×100 mm, 3.0 μm; eluent: tert-butyl methyl ether (containing 0.1% diethylamine)/i-PrOH—60:40). 20 mg (8%) as a colorless solid. HPLC/MS m/z: 509.30 [M+H]⁺, Rt (L): 1.07 min. ¹H NMR (300 MHz, DMSO-d₆): δ 12.56 (s, 1H), 8.73 (d, J=7.8 Hz, 1H), 8.18 (s, 1H), 7.96 (t, J=1.8 Hz, 1H), 7.90 (d, J=0.8 Hz, 1H), 7.74 (dt, J=7.7, 1.5 Hz, 1H), 7.63 (dt, J=7.8, 1.4 Hz, 1H), 7.47 (t, J=7.7 Hz, 1H), 4.67 (q, J=6.7 Hz, 1H), 3.88 (s, 3H), 3.02-2.84 (m, 4H), 2.52 (s, 3H), 1.50 (s, 9H). Chiral HPLC: Rt: 2.58 min.

### Example 202: tert-Butyl 2-[(3R)-4-cyano-3-{[3-(1-methyl-1 H-pyrazol-4-yl)phenyl]formamidolbutanamido]-4-methyl-1,3-thiazole-5-carboxylate

19 mg (8%) as a colorless solid. HPLC/MS m/z: 509.30 [M+H]⁺, Rt (L): 1.07 min. ¹H NMR (300 MHz, DMSO-d₆): δ 12.56 (s, 1H), 8.73 (d, J=7.8 Hz, 1H), 8.18 (s, 1H), 7.96 (t, J=1.8 Hz, 1H), 7.90 (d, J=0.8 Hz, 1H), 7.74 (dt, J=7.8, 1.5 Hz, 1H), 7.63 (dt, J=7.8, 1.4 Hz, 1H), 7.47 (t, J=7.7 Hz, 1H), 4.67 (q, J=6.6 Hz, 1H), 3.88 (s, 3H), 3.04-2.82 (m, 4H), 2.52 (s, 3H), 1.50 (s, 9H). Chiral HPLC: Rt: 3.15 min.

### Example 203: Propan-2-yl 2-[(3S)-4-cyano-3-{[3-(1-methyl-1 H-pyrazol-4-yl)phenyl]formamidolbutanamido]-4-methyl-1,3-thiazole-5-carboxylate

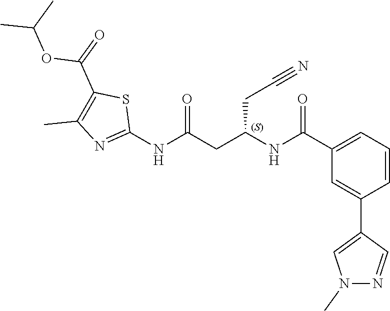

Separation of the enantiomers was performed by preparative chiral HPLC (column: CHIRALPAK ID-3, 4.6×50 mm, 3 μm, eluent: tert-butyl methyl ether (containing 0.1% diethylamine)/i-PrOH—70:30).

22 mg (9%) as a colorless solid. HPLC/MS m/z: 495.30 [M+H]⁺, Rt (L): 1.06 min. ¹H NMR (300 MHz, DMSO-d₆): δ 12.62 (s, 1H), 8.73 (d, J=7.8 Hz, 1H), 8.18 (s, 1H), 7.96 (t, J=1.8 Hz, 1H), 7.89 (d, J=0.8 Hz, 1H), 7.74 (dt, J=7.7, 1.5 Hz, 1H), 7.63 (dt, J=7.8, 1.4 Hz, 1H), 7.46 (t, J=7.7 Hz, 1H), 5.04 (sept, J=6.2 Hz, 1H), 4.74-4.61 (m, 1H), 3.88 (s, 3H), 3.03-2.84 (m, 4H), 2.54 (s, 3H), 1.27 (d, J=6.3 Hz, 6H). Chiral HPLC: Rt: 2.07 min.

### Example 204: Propan-2-yl 2-[(3R)-4-cyano-3-{[3-(1-methyl-1 H-pyrazol-4-yl)phenyl]formamidolbutanamido]-4-methyl-1,3-thiazole-5-carboxylate

20.5 mg (9%) as a colorless solid. HPLC/MS m/z: 495.30 [M+H]⁺, Rt (L): 1.05 min. ¹H NMR (300 MHz, DMSO-d₆): δ 12.62 (s, 1H), 8.73 (d, J=7.8 Hz, 1H), 8.18 (s, 1H), 7.96 (t, J=1.8 Hz, 1H), 7.89 (d, J=0.8 Hz, 1H), 7.74 (dt, J=7.7, 1.4 Hz, 1H), 7.63 (dt, J=7.8, 1.3 Hz, 1H), 7.47 (t, J=7.7 Hz, 1H), 5.04 (sept, J=6.2 Hz, 1H), 4.74-4.61 (m, 1H), 3.88 (s, 3H), 3.04-2.82 (m, 4H), 2.54 (s, 3H), 1.27 (d, J=6.2 Hz, 6H). Chiral HPLC: Rt: 3.07 min.

### Example 205: Propan-2-yl 2-[(3S)-4-cyano-N-methyl-3-{[3-(1-methyl-1 H-pyrazol-4-yl)phenyl]formamidolbutanamido]-4-methyl-1,3-thiazole-5-carboxylate

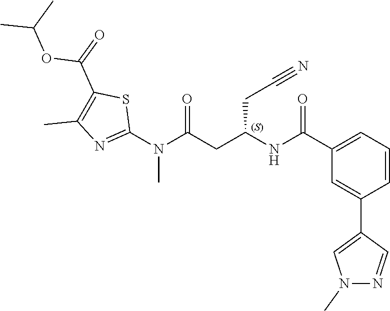

Separation of the enantiomers was performed by preparative chiral HPLC (column: CHIRALPAK ID-3, 4.6×50 mm, 3 μm, eluent: tert-butyl methyl ether (containing 0.1% diethylamine)/i-PrOH—70:30).

67 mg (14%) as a colorless solid. HPLC/MS m/z: 509.05 [M+H]⁺, Rt (J): 1.01 min. ¹H NMR (300 MHz, DMSO-d₆): δ 8.71 (d, J=7.5 Hz, 1H), 8.19 (s, 1H), 7.97 (t, J=1.7 Hz, 1H), 7.89 (d, J=0.8 Hz, 1H), 7.79-7.70 (m, 1H), 7.68-7.59 (m, 1H), 7.47 (t, J=7.7 Hz, 1H), 5.05 (sept, J=6.2 Hz, 1H), 4.73-4.63 (m, 1H), 3.88 (s, 3H), 3.68 (s, 3H), 3.32-3.09 (m, 2H), 3.09-2.97 (m, 1H), 2.97-2.83 (m, 1H), 2.57 (s, 3H), 1.27 (dd, J=6.2, 1.2 Hz, 6H). Chiral HPLC: Rt: 1.47 min.

### Example 206: Propan-2-yl 2-[(3R)-4-cyano-N-methyl-3-{[3-(1-methyl-1 H-pyrazol-4-yl)phenyl]formamidolbutanamido]-4-methyl-1,3-thiazole-5-carboxylate

56 mg (12%) as a colorless solid. HPLC/MS m/z: 509.05 [M+H]⁺, Rt (J): 1.01 min. ¹H NMR (300 MHz, DMSO-d₆): δ 8.71 (d, J=7.5 Hz, 1H), 8.19 (s, 1H), 7.97 (t, J=1.7 Hz, 1H), 7.89 (d, J=0.8 Hz, 1H), 7.79-7.70 (m, 1H), 7.69-7.59 (m, 1H), 7.47 (t, J=7.7 Hz, 1H), 5.05 (sept, J=6.2 Hz, 1H), 4.75-4.63 (m, 1H), 3.88 (s, 3H), 3.68 (s, 3H), 3.32-3.05 (m, 2H), 3.10-2.97 (m, 1H), 2.97-2.83 (m, 1H), 2.57 (s, 3H), 1.27 (dd, J=6.2, 1.2 Hz, 6H). Chiral HPLC: Rt: 1.87 min.

### Example 207: Propan-2-yl 2-[(3S)-4-cyano-3-[(3-cyanophenyl)formamido]butan-amido]-4-methyl-1,3-thiazole-5-carboxylate

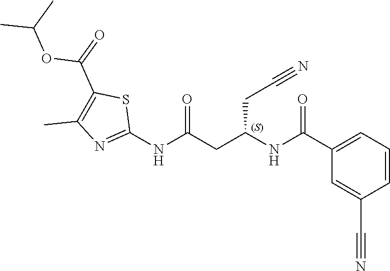

Separation of the enantiomers was performed by preparative chiral HPLC (column: CHIRALPAK IF-3, 4.6×50 mm, 3 μm, eluent: tert-butyl methyl ether (containing 0.1% diethylamine)/EtOH—90:10).

49 mg (11%) as a colorless solid. HPLC/MS m/z: 440.10 [M+H]⁺, Rt (E): 0.82 min. ¹H NMR (400 MHz, DMSO-d₆): δ 12.61 (s, 1H), 8.94 (d, J=7.7 Hz, 1H), 8.24 (t, J=1.7 Hz, 1H), 8.14 (dt, J=8.0, 1.5 Hz, 1H), 8.05 (dt, J=7.8, 1.4 Hz, 1H), 7.73 (t, J=7.8 Hz, 1H), 5.12-4.98 (m, 1H), 4.72-4.62 (m, 1H), 3.03-2.90 (m, 2H), 2.94-2.83 (m, 2H), 2.54 (s, 3H), 1.27 (dd, J=6.2, 1.1 Hz, 6H). Chiral HPLC: Rt: 1.89 min.

### Example 208: Propan-2-yl 2-[(3S)-4-cyano-3-[(7-cyanoisoquinolin-1-yl)amino]butan-amido]-4-methyl-1,3-thiazole-5-carboxylate

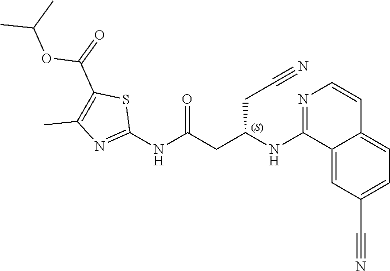

Separation of the enantiomers was performed by preparative chiral HPLC (column: CHIRALPAK IA-3, 4.6×50 mm, 3 μm, eluent: hexane (containing 0.1% diethylamine)/EtOH—50:50). 34 mg (27%) as a colorless solid. HPLC/MS m/z: 463.10 [M+H]⁺, Rt (C): 0.90 min. ¹H NMR (400 MHz, DMSO-d₆): δ 12.61 (s, 1H), 8.90 (s, 1H), 8.08 (d, J=5.8 Hz, 1H), 7.98-7.88 (m, 2H), 7.84 (d, J=7.2 Hz, 1H), 7.08 (d, J=5.8 Hz, 1H), 5.09-5.00 (m, 1H), 5.03-4.95 (m, 1H), 3.16-3.04 (m, 2H), 3.03-2.89 (m, 2H), 2.53 (s, 3H), 1.26 (dd, J=6.3, 2.0 Hz, 6H). Chiral HPLC: Rt: 2.83 min.

### Example 209: Propan-2-yl 2-[(3R)-4-cyano-3-[(7-cyanoisoquinolin-1-yl)amino]butan-amido]-4-methyl-1,3-thiazole-5-carboxylate

38 mg (29%) as a colorless solid. HPLC/MS m/z: 463.10 [M+H]⁺, Rt (C): 0.90 min. ¹H NMR (400 MHz, DMSO-d₆): δ 12.61 (s, 1H), 8.90 (d, J=1.4 Hz, 1H), 8.08 (d, J=5.8 Hz, 1H), 7.98-7.88 (m, 2H), 7.84 (d, J=7.2 Hz, 1H), 7.08 (d, J=5.7 Hz, 1H), 5.10-5.00 (m, 1H), 5.02-4.93 (m, 1H), 3.17-3.06 (m, 2H), 3.02-2.91 (m, 2H), 2.53 (s, 3H), 1.26 (dd, J=6.3, 2.0 Hz, 6H). Chiral HPLC: Rt: 1.25 min.

### Example 210: Propan-2-yl 2-[(3S)-4-cyano-3-{[7-(1-methyl-1 H-pyrazol-4-yl)isoquinolin-1-yl]amino}butanamido]-4-methyl-1,3-thiazole-5-carboxylate

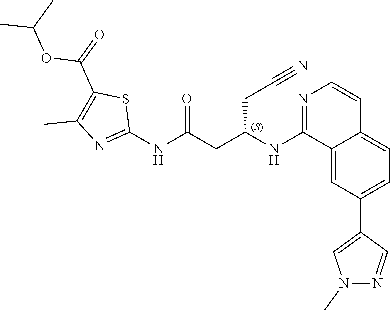

Separation of the enantiomers was performed by preparative chiral HPLC (column: (R,R) WHELK-01, 4.6×50 mm, 3.5 μm, eluent: hexane (containing 0.1% diethylamine)/EtOH—50:50).

15 mg (9%) as a colorless solid. HPLC/MS m/z: 518.15 [M+H]⁺, Rt (D): 1.52 min. ¹H NMR (300 MHz, DMSO-d₆): δ 12.66 (brs, 1H), 8.40 (s, 1H), 8.23 (s, 1H), 8.03 (d, J=0.8 Hz, 1H), 7.92-7.81 (m, 2H), 7.74 (d, J=8.5 Hz, 1H), 7.44 (d, J=7.4 Hz, 1H), 6.97 (d, J=5.8 Hz, 1H), 5.11-4.90 (m, 2H), 3.92 (s, 3H), 3.19-3.07 (m, 2H), 3.06-2.93 (m, 2H), 2.53 (s, 3H), 1.26 (dd, J=6.2, 1.8 Hz, 6H). Chiral HPLC: Rt: 2.51 min.

### Example 211: tert-Butyl 2-[(3S)-4-cyano-3-{[6-(1-methyl-1 H-pyrazol-4-yl)pyridin-2-yl]formamido}butanamido]-4-methyl-1,3-thiazole-5-carboxylate

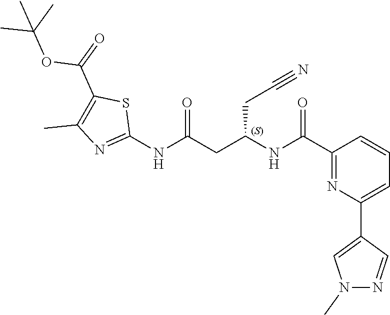

Separation of the enantiomers was performed by preparative chiral HPLC (column: CHIRALPAK ID-3, 4.6×50 mm, 3 μm, eluent: (hexane/dichloromethane—3:1, containing 0.1% diethylamine)/i-PrOH—50:50).

6 mg (10%) as a colorless solid. HPLC/MS m/z: 510.15 [M+H]⁺, Rt (L): 1.05 min. ¹H NMR (400 MHz, DMSO-d₆): δ 12.61 (s, 1H), 9.03 (d, J=9.1 Hz, 1H), 8.46 (s, 1H), 8.27 (s, 1H), 7.95 (t, J=7.8 Hz, 1H), 7.82 (ddd, J=17.3, 7.8, 1.1 Hz, 2H), 4.76 (q, J=7.4, 6.7 Hz, 1H), 3.93 (s, 3H), 3.10-2.89 (m, 4H), 2.53-2.51 (m, 3H), 1.50 (s, 9H). Chiral HPLC: Rt: 1.42 min.

### Example 212: tert-Butyl 2-[(3R)-4-cyano-3-{[6-(1-methyl-1 H-pyrazol-4-yl)pyridin-2-yl]formamido}butanamido]-4-methyl-1,3-thiazole-5-carboxylate

7 mg (10%) as a colorless solid. HPLC/MS m/z: 510.20 [M+H]⁺, Rt (L): 1.05 min. ¹H NMR (400 MHz, DMSO-d₆): δ 12.61 (s, 1H), 9.03 (d, J=9.0 Hz, 1H), 8.46 (s, 1H), 8.27 (s, 1H), 7.95 (t, J=7.7 Hz, 1H), 7.82 (ddd, J=17.2, 7.8, 1.1 Hz, 2H), 4.82-4.69 (m, 1H), 3.93 (s, 3H), 3.10-2.89 (m, 4H), 2.54-2.51 (m, 3H), 1.50 (s, 9H). Chiral HPLC: Rt: 2.82 min.

### Example 213: Propan-2-yl 2-[(3S)-4-cyano-3-{[6-(1-methyl-1 H-pyrazol-4-yl)pyridin-2-yl]formamido}butanamido]-4-methyl-1,3-thiazole-5-carboxylate

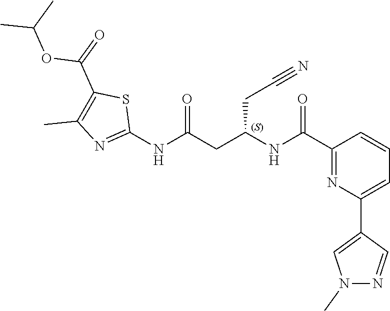

Separation of the enantiomers was performed by preparative chiral HPLC (column: CHIRALPAK IA-3, 4.6×50 mm, 3 μm, eluent: hexane (containing 0.1% diethylamine)/EtOH—60:40). 24 mg (22%) as a colorless solid. HPLC/MS m/z: 496.10 [M+H]⁺, Rt (C): 0.83 min. ¹H NMR (400 MHz, DMSO-d₆): δ 12.66 (s, 1H), 9.04 (d, J=9.0 Hz, 1H), 8.46 (s, 1H), 8.27 (d, J=0.7 Hz, 1H), 7.95 (t, J=7.8 Hz, 1H), 7.88-7.76 (m, 2H), 5.10-5.00 (m, 1H), 4.83-4.71 (m, 1H), 3.93 (s, 3H), 3.10-2.88 (m, 4H), 2.55-2.52 (m, 3H), 1.27 (dd, J=6.2, 1.7 Hz, 6H). Chiral HPLC: Rt: 2.00 min.

### Example 214: Propan-2-yl 2-[(3R)-4-cyano-3-{[6-(1-methyl-1 H-pyrazol-4-yl)pyridin-2-yl]form-amidolbutanamido]-4-methyl-1,3-thiazole-5-carboxylate

25 mg (22%) as a colorless solid. HPLC/MS m/z: 496.10 [M+H]⁺, Rt (C): 0.83 min. ¹H NMR (400 MHz, DMSO-d₆): δ 12.66 (s, 1H), 9.04 (d, J=9.0 Hz, 1H), 8.46 (s, 1H), 8.27 (s, 1H), 7.95 (t, J=7.8 Hz, 1H), 7.88-7.76 (m, 2H), 5.12-4.98 (m, 1H), 4.81-4.69 (m, 1H), 3.93 (s, 3H), 3.10-2.88 (m, 4H), 2.56-2.52 (m, 3H), 1.27 (dd, J=6.2, 1.8 Hz, 6H). Chiral HPLC: Rt: 3.57 min.

### Example 215: Ethyl 4-methyl-2-(3-{[3-(5-methyl-1,2,4-oxadiazol-3-yl)phenyl]form-amidolpentanamido)-1,3-thiazole-5-carboxylate

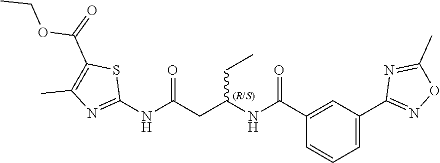 [SMILES: CCOC(=O)c1sc(NC(=O)CC(CC)NC(=O)c2cccc(-c3noc(C)n3)c2)nc1C]

30 mg (12%) as a colorless solid. HPLC/MS m/z: 472.20 [M+H]⁺, Rt (N): 1.47 min. ¹H NMR (300 MHz, DMSO-d₆): δ 12.51 (s, 1H), 8.50 (d, J=8.5 Hz, 1H), 8.41 (t, J=1.6 Hz, 1H), 8.10 (dt, J=7.8, 1.4 Hz, 1H), 7.99 (dt, J=8.0, 1.3 Hz, 1H), 7.63 (t, J=7.8 Hz, 1H), 4.36 (q, J=7.1 Hz, 1H), 4.20 (q, J=7.1 Hz, 2H), 2.82-2.61 (m, 2H), 2.67 (s, 3H), 2.51 (s, 3H), 1.62-1.56 (m, 2H), 1.24 (t, J=7.1 Hz, 3H), 0.87 (t, J=7.3 Hz, 3H).

### Example 216: (3R)-4-{[5-(methoxycarbonyl)-4-methyl-1,3-thiazol-2-yl]carbamoyl}-3-{[3-(5-methyl-1,2,4-oxadiazol-3-yl)phenyl]formamido}butanoic acid

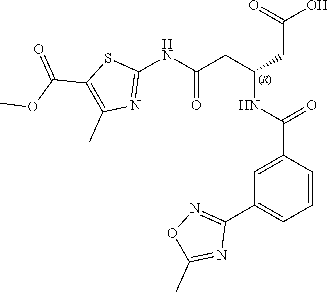

15 mg (19%) as a colorless solid. HPLC/MS m/z: 487.95 [M+H]⁺, Rt (O): 1.02 min. ¹H NMR (300 MHz, DMSO-d₆): δ 12.5 (s, 1H), 8.70 (d, J=8.1 Hz, 1H), 8.40 (t, J=1.8 Hz, 1H), 8.20-8.06 (m, 1H), 7.97 (dt, J=7.9, 1.5 Hz, 1H), 7.64 (t, J=7.8 Hz, 1H), 4.72 (d, J=5.6 Hz, 1H), 3.74 (s, 3H), 2.81 (d, J=6.7 Hz, 2H), 2.67 (s, 3H), 2.61 (dd, J=9.1, 7.0 Hz, 2H), 2.51 (s, 3H).

### Example 217: (3S)-4-{[5-(methoxycarbonyl)-4-methyl-1,3-thiazol-2-yl](methyl)-carbamoyl}-3-{[3-(5-methyl-1,2,4-oxadiazol-3-yl)phenyl]formamidolbutanoic acid

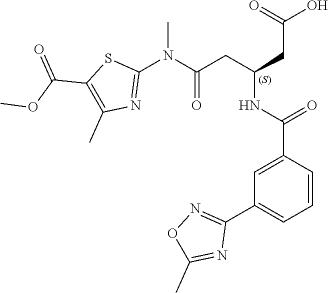

33 mg (16%) as a colorless solid. HPLC/MS m/z: 502.15 [M+H]⁺, Rt (N): 0.83 min. ¹H NMR (400 MHz, DMSO-d₆): δ 8.79 (d, J=7.7 Hz, 1H), 8.40 (t, J=1.7 Hz, 1H), 8.16-8.09 (m, 1H), 8.02-7.95 (m, 1H), 7.65 (t, J=7.8 Hz, 1H), 4.80-4.70 (m, 1H), 3.76 (s, 3H), 3.69 (s, 3H), 3.10 (d, J=6.7 Hz, 2H), 2.68 (s, 3H), 2.66-2.62 (m, 2H), 2.56 (s, 3H).

### Example 218: tert-Butyl 2-((1s,3s)-N-ethyl-3-((7-(5-methyl-1,2,4-oxadiazol-3-yl)isoquinolin-1-yl)amino)cyclobutanecarboxamido)-4-methylthiazole-5-carboxylate formate

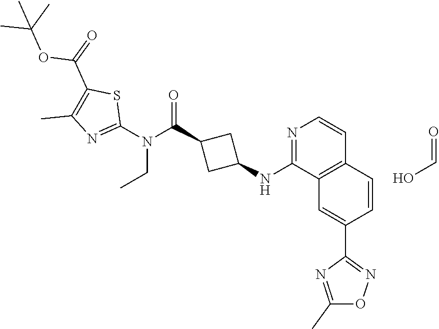

Example 218.1: To a solution of tert-butyl acetoacetate (0.80 mL, 4.7998 mmol) in H₂O (24.00 mL) held at 70° C. was added 1,3,5-tribromo-1,3,5-triazinane-2,4,6-trione (702.24 mg, 1.9199 mmol) in small portions and the mixture was stirred at 70° C. for 1 h. Then, MeCN (24.00 mL), ethylthiourea (500.00 mg, 4.7998 mmol), and DABCO (538.41 mg, 4.7998 mmol) were successively added, and the mixture was stirred at 70° C. for an additional 3 h. After the completion of the reaction, the mixture was poured onto ice and the precipitated solid was filtered. The filtrate was concentrated under reduced pressure and the crude mixture was subjected to column chromatography (10-80% EtOAc:cyclohexane). The fractions were collected and concentrated under reduced pressure to afford tert-butyl 2-(ethylamino)-4-methyl-thiazole-5-carboxylate (104.2 mg, 9%, 0.4300 mmol) as a yellow solid. HPLC/MS m/z 187.0539 [M+H]⁺, Rt (R): 1.36 min. ¹H NMR (600 MHz, Chloroform-d) δ 3.27 (q, J=7.2 Hz, 2H), 2.49 (s, 3H), 1.53 (s, 9H), 1.29 (t, J=7.2 Hz, 3H).

Example 218.2: Preparation as described for example Example 48 using 3-[[7-(5-methyl-1,2,4-oxadiazol-3-yl)-1-isoquinolyl]amino]cyclobutanecarboxylic acid [Example 96](40.00 mg, 0.1233 mmol) and tert-butyl 2-(ethylamino)-4-methyl-thiazole-5-carboxylate (32.88 mg, 0.1357 mmol). Purification by reverse flash chromatography followed by SCX-2 filtration. Compound was subjected to normal column chromatography (0-20% MeOH:DCM). Compound was again purified by reverse phase flash chromatography (5-80% MeOH in water [0.1% FA]). Fractions containing product were dried to afford tert-butyl 2-((1s,3s)-N-ethyl-3-((7-(5-methyl-1,2,4-oxadiazol-3-yl)isoquinolin-1-yl)amino)cyclobutanecarboxamido)-4-methylthiazole-5-carboxylate formate. Yield: 22 mg (33%) colourless, amorphous solid. HPLC/MS m/z 549.2270 [M+H]⁺, Rt (S): 3.07 min. ¹H NMR (600 MHz, DMSO-d₆) δ 9.01 (d, J=1.6 Hz, 1H), 8.15 (dd, J=8.5, 1.5 Hz, 1H), 8.10 (d, J=7.0 Hz, 1H), 7.97 (d, J=5.7 Hz, 1H), 7.85 (d, J=8.5 Hz, 1H), 6.98 (d, J=5.7 Hz, 1H), 4.83-4.73 (m, 1H), 4.17 (q, J=7.0 Hz, 2H), 3.54-3.45 (m, 1H), 2.71 (s, 4H), 2.55 (s, 3H), 2.50 (dt, J=11.7, 9.0 Hz, 2H), 1.52 (s, 9H), 1.29 (t, J=7.0 Hz, 3H).

### Example 219: Ethyl 4-methyl-2-(N-methyl-3-(3-(5-methyl-1,2,4-oxadiazol-3-yl)benzamido)propanamido)thiazole-5-carboxylate

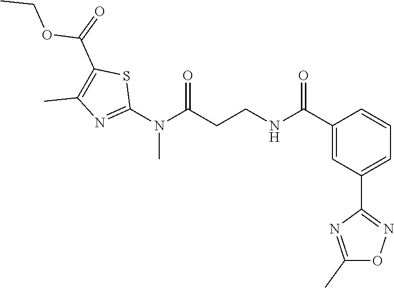 [SMILES: CCOC(=O)c1sc(N(C)C(=O)CCNC(=O)c2cccc(-c3noc(C)n3)c2)nc1C]

Example 219.1: To a stirred solution of 3-(tert-butoxycarbonylamino)propanoic acid (430.00 mg, 2.2726 mmol), and HOBt (614.15 mg, 4.5452 mmol) in dry DMF (11.36 mL), under a nitrogen atmosphere at rt, were added EDC (705.60 mg, 4.5452 mmol) and ethyl 4-methyl-2-(methylamino)thiazole-5-carboxylate (540.00 mg, 2.6965 mmol) sequentially. The mixture was stirred at 60° C. for 18 h. The reaction mixture was partitioned between water (50 mL) and EtOAc (40 mL). The organic layer was washed with water (40 mL), aqueous saturated bicarbonate (20 mL), and brine (20 mL), dried over sodium sulfate, and concentrated in vacuo to give ethyl 2-[3-(tert-butoxycarbonylamino)propanoyl-methyl-amino]-4-methyl-thiazole-5-carboxylate (798 mg, 95%, 2.1483 mmol) as a yellow solid. HPLC/MS m/z: 394.1 [M+Na]⁺, Rt (R): 1.54 min.

Example 219.2: To ethyl 2-[3-(tert-butoxycarbonylamino)propanoyl-methyl-amino]-4-methyl-thiazole-5-carboxylate (400.00 mg, 1.0769 mmol) in dioxane (5.38 mL), was added 4M HCl in dioxane (5.38 mL, 21.537 mmol) dropwise, whilst stirring at rt. After 2 h volatiles were removed under vaccum to afford ethyl 2-[3-aminopropanoyl(methyl)amino]-4-methyl-thiazole-5-carboxylate. Taken on straight away to the next step to avoid decomposition. HPLC/MS m/z: 272.1 [M+H]⁺, Rt (P): 0.94 min.

Example 219.3: To ethyl 2-[3-aminopropanoyl(methyl)amino]-4-methyl-thiazole-5-carboxylate (292.00 mg, 1.0761 mmol), 3-(5-methyl-1,2,4-oxadiazol-3-yl)benzoic acid (219.73 mg, 1.0761 mmol) in DMF (10.76 mL) was added DIPEA (751.80 uL, 4.3046 mmol) followed by HATU (379.77 mg, 1.6142 mmol). The reaction was stirred at rt overnight. The mixture was diluted with EtOAc and washed with water, aqueous saturated bicarbonate, and brine. The organic layer was dried over magnesium sulfate and concentrated in vacuo to afford a yellow crude product (469 mg). 90 mg was purified by reverse phase column chomatography (eluent: 30-80% MeOH in water (+0.1% formic acid modifier in both)) to afford ethyl 4-methyl-2-(N-methyl-3-(3-(5-methyl-1,2,4-oxadiazol-3-yl)benzamido)propanamido)thiazole-5-carboxylate (40 mg, 8%, 0.0874 mmol) as a colorless power. HPLC/MS m/z: 458.1 [M+H]⁺, Rt (S): 3.23 min. ¹H NMR (500 MHz, DMSO-d6) δ 8.83 (t, J=5.4 Hz, 1H), 8.48 (td, J=1.8, 0.6 Hz, 1H), 8.17-8.11 (m, 1H), 8.05 (ddd, J=7.8, 1.8, 1.1 Hz, 1H), 7.71-7.64 (m, 1H), 4.25 (q, J=7.1 Hz, 2H), 3.69-3.59 (m, 5H), 3.08 (t, J=6.8 Hz, 2H), 2.69 (s, 3H), 2.58 (s, 3H), 1.29 (t, J=7.1 Hz, 3H).

### Example 220: Propyl 4-methyl-2-(((1s,3s)-3-((7-(5-methyl-1,2,4-oxadiazol-3-yl)isoquinolin-1-yl)amino)cyclobutyl)carbamoyl)thiazole-5-carboxylate

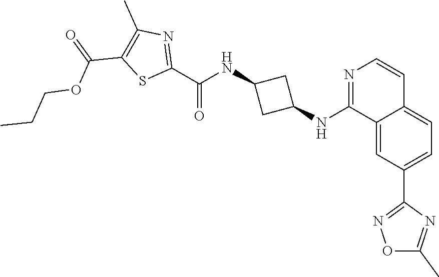 [SMILES: CCCOC(=O)c1sc(C(=O)N[C@H]2C[C@@H](Nc3nccc4ccc(-c5noc(C)n5)cc34)C2)nc1C]

Example 220.1: 5-Methyl-3-(2-oxidoisoquinolin-2-ium-7-yl)-1,2,4-oxadiazole [Example 96](375 mg, 1.65 mmol), tert-butyl (cis-3-aminocyclobutyl)carbamate (384 mg, 2.06 mmol) and DIPEA (1.08 mL, 6.19 mmol) were mixed in anhydrous DCM (6.6 mL) at rt under an argon atmosphere. PyBroP (1.00 g, 2.15 mmol) was added to the suspension and the mixture was stirred at rt overnight. The reaction mixture was concentrated under reduced pressure and purified by flash chromatography (20-90% EtOAc in cyclohexane) to yield 431 mg (66%) of tert-butyl N-[3-[[7-(5-methyl-1,2,4-oxadiazol-3-yl)-1-isoquinolyl]amino]cyclobutyl]carbamate as a colourless, amorphous solid. HPLC/MS m/z: 396.2035 [M+H]⁺, Rt (Q): 2.43 min.

Example 220.2: tert-Butyl N-[3-[[7-(5-methyl-1,2,4-oxadiazol-3-yl)-1-isoquinolyl]amino]cyclobutyl]carbamate (431 mg, 1.09 mmol) was dissolved in anhydrous DCM (2.2 mL) at rt under an argon atmosphere. Trifluoroacetic acid (0.83 mL, 10.9 mmol) was added, and the reaction mixture was stirred for 3 h. Volatiles were removed under reduced pressure. The crude material was dissolved in MeOH and filtered through a 2 g SCX-2 cartridge. The compound was released with 7 M ammonia in MeOH. Volatiles were removed under reduced pressure to yield 315 mg (98%) of N1-[7-(5-methyl-1,2,4-oxadiazol-3-yl)-1-isoquinolyl]cyclobutane-1,3-diamine as an off-white solid. HPLC/MS m/z: 296.1513 [M+H]⁺, Rt (S): 1.09 min.

Example 220.3: 2-Bromo-4-methyl-thiazole-5-carboxylic acid (0.90 g, 4.05 mmol) and DMAP (0.05 g, 0.405 mmol) were suspended in anhydrous DMF (8.1 mL) at rt under an argon atmosphere. 1-(3-dimethylaminopropyl)-3-ethylcarbodiimide hydrochloride (0.93 g, 4.86 mmol) and propan-1-ol (6.1 mL, 81.06 mmol) were added successively and the reaction mixture was stirred at 60° C. for 4 h. The reaction mixture was cooled to rt and mixed with water (50 mL). The mixture was extracted with EtOAc (3×20 mL). The combined organic layer was washed with 1 M HCl (20 mL) and saturated NaCl (2×20 mL), dried over anhydrous MgSO4 and concentrated under reduced pressure. The crude material was purified by flash chromatography (0-30% EtOAc in cyclohexane) to yield 570 mg (53%) of propyl 2-bromo-4-methyl-thiazole-5-carboxylate as a colourless, crystalline solid. HPLC/MS m/z: 263.9699 [M+H]⁺, Rt (S): 3.19 min.

Example 220.4: N1-[7-(5-methyl-1,2,4-oxadiazol-3-yl)-1-isoquinolyl]cyclobutane-1,3-diamine (30 mg, 0.102 mmol), propyl 2-bromo-4-methyl-thiazole-5-carboxylate (28 mg, 0.107 mmol), XantPhos-Pd G4 precatalyst (4.9 mg, 0.005 mmol) and anhydrous 1,4-dioxane (0.51 mL) were mixed in a microwave vial. The vial was capped, evacuated, and back filled with argon. Then, the vial was evacuated again and fitted with a balloon filled with carbon monoxide. DIPEA (0.07 mL, 0.406 mmol) was added, and the reaction mixture was heated at 80° C. for 3 h. The reaction mixture was cooled to rt and volatiles were removed under reduced pressure. The crude material was purified by reverse flash chromatography (20-80% MeOH in water [0.1% FA]). Fractions containing product were filtered through a 1 g SCX-2 cartridge. The compound was released with 7 M ammonia in MeOH. The solvent was removed under reduced pressure to yield 22 mg (41%) of propyl 4-methyl-2-(((1s,3s)-3-((7-(5-methyl-1,2,4-oxadiazol-3-yl)isoquinolin-1-yl)amino)cyclobutyl)carbamoyl)thiazole-5-carboxylate as off-white, amorphous solid. HPLC/MS m/z: 507.1814 [M+H]⁺, Rt (Q): 2.72 min. ¹H NMR (600 MHz, DMSO-d₆) b 9.15 (d, J=7.4 Hz, 1H), 8.98 (s, 1H), 8.15 (dd, J=8.5, 1.5 Hz, 1H), 7.96 (d, J=5.7 Hz, 1H), 7.93 (d, J=6.2 Hz, 1H), 7.85 (d, J=8.5 Hz, 1H), 6.97 (d, J=5.7 Hz, 1H), 4.40-4.33 (m, 1H), 4.23 (t, J=6.5 Hz, 2H), 4.22-4.15 (m, 1H), 2.81-2.75 (m, 2H), 2.73-2.70 (m, 6H), 2.38-2.31 (m, 2H), 1.75-1.67 (m, 2H), 0.96 (t, J=7.4 Hz, 3H).

### Example 221: (1 r,3r)-N-(5-(tert-butyl)-4-methylthiazol-2-yl)-1-hydroxy-3-((7-(5-methyl-1,2,4-oxadiazol-3-yl)isoquinolin-1-yl)amino)cyclobutane-1-carboxamide

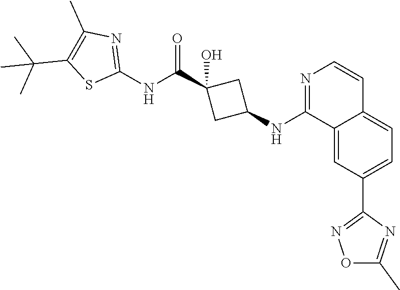 [SMILES: Cc1nc(-c2ccc3ccnc(N[C@H]4C[C@@](O)(C(=O)Nc5nc(C)c(C(C)(C)C)s5)C4)c3c2)no1]

Example 221.1: tert-Butyl 3-oxocyclobutylcarbamate (500 mg, 2.70 mmol) and zinc iodide (43 mg, 0.14 mmol) were mixed at rt under an argon atmosphere. Anhydrous THF (13.50 mL) was added. Trimethylsilylcyanide (0.41 mL, 3.24 mmol) was added, and the reaction mixture was stirred at rt for 60 h. The reaction mixture was concentrated under reduced pressure and mixed with EtOAc (50 mL), washed with saturated NaHCO₃ (20 mL) and saturated NaCl (2×20 mL), dried over anhydrous MgSO₄ and concentrated under reduced pressure to yield 554 mg (97%) of the crude tert-butyl N-(3-cyano-3-hydroxy-cyclobutyl)carbamate, which was used in the next reaction without further purification. Note: ¹H NMR showed a −2:3 mixture of both stereoisomers.

Example 221.2: tert-Butyl N-(3-cyano-3-hydroxy-cyclobutyl)carbamate (554 mg, 2.61 mmol) was heated to reflux in a 1:1 mixture of concentrated HCl/AcOH (20 mL) for 2 h. The reaction mixture was cooled to rt and concentrated under reduced pressure to yield 420 mg (96%) of (3-carboxy-3-hydroxy-cyclobutyl)ammonium chloride as an off-white solid, which was used in the next reaction without further purification. HPLC/MS m/z: 132.0661 [M+H]⁺, Rt (S): 0.31 min. Note: ¹H NMR showed a ˜1:4 mixture of both stereoisomers.

Example 221.3: 3-Amino-1-hydroxycyclobutane-1-carboxylic acid hydrochloride (360 mg, 2.15 mmol), sodium carbonate (759 mg, 7.16 mmol), and 3-(1-chloro-7-isoquinolyl)-5-methyl-1,2,4-oxadiazole [Example 45](352 mg, 1.43 mmol) were mixed in anhydrous NMP (2.9 mL) under an argon atmosphere. The reaction mixture was heated at 130° C. for 48 h. The reaction mixture was cooled to ambient temperature and concentrated under reduced pressure. The crude material was purified by reverse flash chromatography (5-40% MeOH in water [0.1% FA]) to yield 89 mg (18%) of 1-hydroxy-3-[[7-(5-methyl-1,2,4-oxadiazol-3-yl)-1-isoquinolyl]amino]cyclobutanecarboxylic acid as an off-white solid. HPLC/MS m/z: 341.1242 [M+H]⁺, Rt (S): 1.74 min. Note: ¹H NMR showed a ˜1:3 mixture of both stereoisomers.

Example 221.4: 5-tert-Butyl-4-methyl-thiazol-2-ylamine (76 mg, 0.45 mmol) and 1-hydroxy-3-[[7-(5-methyl-1,2,4-oxadiazol-3-yl)-1-isoquinolyl]amino]cyclobutane-carboxylic acid (78 mg, 0.22 mmol) were mixed in anhydrous DMF (0.46 mL) at 0° C. under an argon atmosphere. PyBOP (140 mg, 0.27 mmol) and DIPEA (0.12 mL, 0.67 mmol) were added, and the stirred reaction mixture was allowed to slowly reach ambient temperature. The reaction was continued to stir overnight. The reaction mixture was directly purified by reverse flash chromatography (10-80% MeOH in water [0.1% FA]) separating the two stereoisomers. The trans-isomer was filtered through a 1 g SCX-2 cartridge. The compound was released with 7 M ammonia in MeOH. Volatiles were removed under reduced pressure to yield 33 mg (30%) of (1s,3s)-N-(5-(tert-butyl)-4-methylthiazol-2-yl)-1-hydroxy-3-((7-(5-methyl-1,2,4-oxadiazol-3-yl)isoquinolin-1-yl)amino)cyclobutane-1-carboxamide as a colorless solid. HPLC/MS m/z: 493.2007 [M+H]⁺, Rt (S): 2.83 min. ¹H NMR (600 MHz, DMSO-d₆) δ 10.75 (br s, 1H), 9.02 (s, 1H), 8.15 (dd, J=8.5, 1.5 Hz, 1H), 8.06 (d, J=6.4 Hz, 1H), 7.95 (d, J=5.7 Hz, 1H), 7.84 (d, J=8.5 Hz, 1H), 6.96 (d, J=5.7 Hz, 1H), 6.45 (s, 1H), 4.59-4.51 (m, 1H), 2.93-2.88 (m, 2H), 2.71 (s, 3H), 2.43-2.38 (m, 2H), 2.33 (s, 3H), 1.39 (s, 9H). The cis-isomer was further purified by prep-HPLC followed by SCX-2 filtration to yield 7.4 mg (8%) of (1 r,3r)-N-(5-(tert-butyl)-4-methylthiazol-2-yl)-1-hydroxy-3-((7-(5-methyl-1,2,4-oxadiazol-3-yl)isoquinolin-1-yl)amino)cyclobutane-1-carboxamide as colorless solid. HPLC/MS m/z: 493.2014 [M+H]⁺, Rt (S): 2.98 min. ¹H NMR (600 MHz, DMSO-d₆) δ 10.91 (br s, 1H), 9.00 (s, 1H), 8.15 (dd, J=8.5, 1.5 Hz, 1H), 8.09 (d, J=6.7 Hz, 1H), 7.99 (d, J=5.7 Hz, 1H), 7.85 (d, J=8.5 Hz, 1H), 6.98 (d, J=5.7 Hz, 1H), 6.18 (s, 1H), 5.01-4.91 (m, 1H), 2.73-2.68 (m, 5H), 2.49-2.43 (m, 2H), 2.33 (s, 3H), 1.37 (s, 9H).

### Example 222: tert-Butyl 2-((1s,3s)-1-fluoro-3-((7-(5-methyl-1,2,4-oxadiazol-3-yl)isoquinolin-1-yl)amino)cyclobutane-1-carboxamido)-4-methylthiazole-5-carboxylate

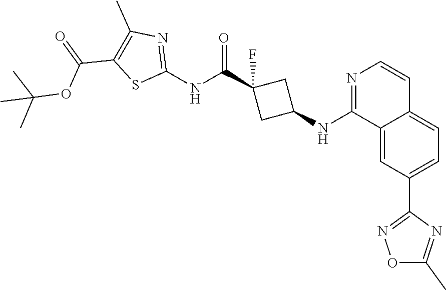 [SMILES: Cc1nc(-c2ccc3ccnc(N[C@H]4C[C@@](F)(C(=O)Nc5nc(C)c(C(=O)OC(C)(C)C)s5)C4)c3c2)no1]

Example 222.1: Acetyl chloride (0.12 mL, 1.62 mmol) was mixed with anhydrous MeOH (3.0 mL) at 0° C. under an argon atmosphere and stirred for 10 min. 3-Amino-1-fluorocyclobutane-1-carboxylic acid hydrochloride (250 mg, 1.47 mmol) was added and the reaction mixture was allowed to warm to ambient temperature and continued to stir overnight. The reaction mixture was concentrated under reduced pressure to yield 261 mg (96%) of methyl 3-amino-1-fluoro-cyclobutanecarboxylate hydrochloride as a colorless solid. HPLC/MS m/z: 148.0769 [M+H]⁺, Rt (U): 0.48 min. Note: ¹H NMR showed a ˜1:3.5 mixture of both stereoisomers.

Example 222.2: Preparation as described for intermediate Example 98.1, using 5-methyl-3-(2-oxidoisoquinolin-2-ium-7-yl)-1,2,4-oxadiazole [Example 96](130 mg, 0.572 mmol) and methyl 3-amino-1-fluoro-cyclobutanecarboxylate hydrochloride (131 mg, 0.715 mmol). Purification by flash chromatography. Yield: 139 mg (68%) off-white, amorphous solid. HPLC/MS m/z: 357.1353 [M+H]⁺, Rt (U): 1.96 and 2.02 min. Note: ¹H NMR showed a ˜1:3.5 mixture of both stereoisomers.

Example 222.3: Preparation as described for intermediate Example 98.2, using methyl 1-fluoro-3-[[7-(5-methyl-1,2,4-oxadiazol-3-yl)-1-isoquinolyl]amino]cyclobutanecarboxylate (131 mg, 0.368 mmol). Purification by reverse flash chromatography. Yield: 100 mg (79%) off-white solid, a ˜1:3.5 mixture of both stereoisomers according to ¹H NMR. HPLC/MS m/z: 343.1194 and 343.1190 [M+H]⁺, Rt (U): 1.81 and 1.88 min, respectively.

Example 222.4: Preparation as described for Example 48 using 1-fluoro-3-[[7-(5-methyl-1,2,4-oxadiazol-3-yl)-1-isoquinolyl]amino]cyclobutanecarboxylic acid (90 mg, 0.263 mmol) and tert-butyl 2-amino-4-methyl-thiazole-5-carboxylate [Example 110.1](113 mg, 0.526 mmol). Separation of both stereoisomers by reverse flash chromatography. Yield: 52 mg (36%) off-white, crystalline solid of tert-butyl 2-((1 r,3r)-1-fluoro-3-((7-(5-methyl-1,2,4-oxadiazol-3-yl)isoquinolin-1-yl)amino)cyclobutane-1-carboxamido)-4-methylthiazole-5-carboxylate. HPLC/MS m/z: 539.1866 [M+H]⁺, Rt (U): 3.13 min. ¹H NMR (600 MHz, Chloroform-d) δ 9.97 (br s, 1H), 8.64 (s, 1H), 8.27 (dd, J=8.5, 1.5 Hz, 1H), 8.16 (d, J=5.8 Hz, 1H), 7.78 (d, J=8.5 Hz, 1H), 7.02 (dd, J=5.9, 0.9 Hz, 1H), 6.22 (d, J=7.8 Hz, 1H), 5.17-5.10 (m, 1H), 3.15-3.05 (m, 2H), 3.01-2.92 (m, 2H), 2.73 (s, 3H), 2.63 (s, 3H), 1.56 (s, 9H)

Example 223: tert-Butyl 2-((1s,3s)-1-fluoro-3-((7-(5-methyl-1,2,4-oxadiazol-3-yl)isoquinolin-1-yl)amino)cyclobutane-1-carboxamido)-4-methylthiazole-5-carboxylate was prepared analogously

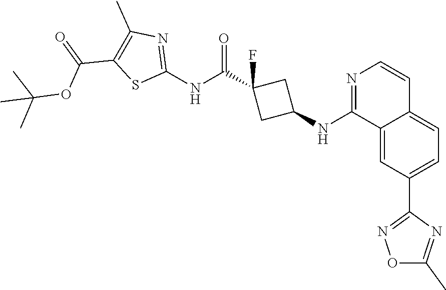 [SMILES: Cc1nc(-c2ccc3ccnc(N[C@H]4C[C@](F)(C(=O)Nc5nc(C)c(C(=O)OC(C)(C)C)s5)C4)c3c2)no1]

Example 223.1: Isolated from reaction mixture in Example 224.4. Yield: 4.5 mg (3%) off-white, amorphous solid of tert-butyl 2-((1s,3s)-1-fluoro-3-((7-(5-methyl-1,2,4-oxadiazol-3-yl)isoquinolin-1-yl)amino)cyclobutane-1-carboxamido)-4-methylthiazole-5-carboxylate. HPLC/MS m/z: 539.1882 [M+H]⁺, Rt (U): 3.08 min. ¹H NMR (600 MHz, Chloroform-d) δ 8.55-8.51 (m, 1H), 8.25 (dd, J=8.5, 1.5 Hz, 1H), 8.21 (s, 1H), 8.06 (d, J=5.8 Hz, 1H), 7.78 (d, J=8.5 Hz, 1H), 7.02-6.99 (m, 1H), 5.69 (br s, 1H), 4.84-4.78 (m, 1H), 3.40-3.33 (m, 2H), 2.73-2.60 (m, 8H), 1.57 (s, 9H).

### Example 224: tert-Butyl 4-methyl-2-((1s,3s)-1-methyl-3-((7-(5-methyl-1,2,4-oxadiazol-3-yl)isoquinolin-1-yl)amino)cyclobutane-1-carboxamido)thiazole-5-carboxylate

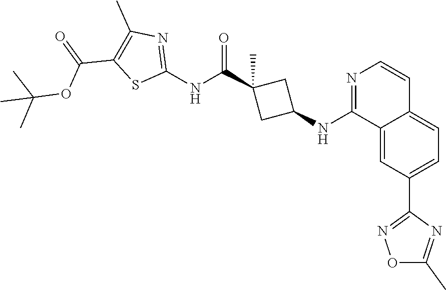 [SMILES: Cc1nc(-c2ccc3ccnc(N[C@H]4C[C@@](C)(C(=O)Nc5nc(C)c(C(=O)OC(C)(C)C)s5)C4)c3c2)no1]

Example 224.1: Acetyl chloride (0.12 mL, 1.66 mmol) was mixed with anhydrous MeOH (3.0 mL) at 0° C. under an argon atmosphere and stirred for 10 min. (1s,3s)-3-Amino-1-methylcyclobutane-1-carboxylic acid hydrochloride (250 mg, 1.51 mmol) was added and the reaction mixture was allowed to warm to ambient temperature and continued to stir overnight. The reaction mixture was concentrated under reduced pressure to yield 271 mg (100%) of methyl (1s,3s)-3-amino-1-methylcyclobutane-1-carboxylate hydrochloride as a colorless solid. HPLC/MS m/z: 144.1029 [M+H]⁺, Rt (U): 0.56 min.

Example 224.2: Preparation as described for intermediate in Example 98.1, using 5-methyl-3-(2-oxidoisoquinolin-2-ium-7-yl)-1,2,4-oxadiazole [Example 96](130 mg, 0.572 mmol) and methyl (1s,3s)-3-amino-1-methylcyclobutane-1-carboxylate hydrochloride (128 mg, 0.715 mmol). Purification by flash chromatography followed by SCX-2 filtration. Yield: 104 mg (52%) off-white, amorphous solid. HPLC/MS m/z: 353.1612 [M+H]⁺, Rt (U): 2.23 min.

Example 224.3: Preparation as described for intermediate in Example 98.2, using methyl (1s,3s)-1-methyl-3-((7-(5-methyl-1,2,4-oxadiazol-3-yl)isoquinolin-1-yl)amino)cyclobutane-1-carboxylate (100 mg, 0.284 mmol). Purification by reverse flash chromatography. Yield: 84 mg (87%) off-white solid. HPLC/MS m/z: 339.1448 [M+H]⁺, Rt (U): 2.03 min.

Example 224.4: Preparation as described for Example 48 using (1s,3s)-1-methyl-3-((7-(5-methyl-1,2,4-oxadiazol-3-yl)isoquinolin-1-yl)amino)cyclobutane-1-carboxylic acid (30 mg, 0.089 mmol) and tert-butyl 2-amino-4-methyl-thiazole-5-carboxylate [Example 110.1](38 mg, 0.177 mmol). Purification by reverse flash chromatography. Yield: 34 mg (71%) off-white solid. HPLC/MS m/z: 535.2120 [M+H]⁺, Rt (U): 3.02 min. ¹H NMR (600 MHz, DMSO-d₆) δ 12.32 (s, 1H), 8.94-8.91 (m, 1H), 8.14 (dd, J=8.6, 1.6 Hz, 1H), 7.99 (d, J=5.7 Hz, 1H), 7.93 (d, J=7.3 Hz, 1H), 7.84 (d, J=8.5 Hz, 1H), 6.97 (d, J=5.7 Hz, 1H), 4.85-4.77 (m, 1H), 2.69 (s, 3H), 2.61-2.55 (m, 2H), 2.53 (s, 3H), 2.43-2.37 (m, 2H), 1.52 (s, 3H), 1.51 (s, 9H).

### Example 225: tert-Butyl 4-methyl-2-[[(3R)-3-[[7-(5-methyl-1,2,4-oxadiazol-3-yl)-1-isoquinolyl]amino]pyrrolidine-1-carbonyl]amino]thiazole-5-carboxylate

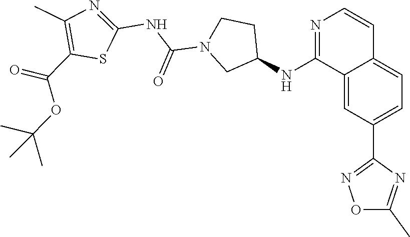 [SMILES: Cc1nc(-c2ccc3ccnc(N[C@@H]4CCN(C(=O)Nc5nc(C)c(C(=O)OC(C)(C)C)s5)C4)c3c2)no1]

Example 225.1: 5-Methyl-3-(2-oxidoisoquinolin-2-ium-7-yl)-1,2,4-oxadiazole [Example 96](187 mg, 0.823 mmol) and (R)-(+)-1-Boc-3-aminopyrrolidine (0.17 mL, 1.029 mmol) were mixed in anhydrous DCM (4.11 mL) at rt under an argon atmosphere. DIPEA (0.54 mL, 3.086 mmol) and PyBroP (499 mg, 1.070 mmol) were added, and the reaction mixture was stirred at rt for 5 h. Volatiles were removed under reduced pressure and the crude material was purified by flash chromatography (10-50% EtOAc in cyclohexane) to yield 179 mg (55%) of tert-butyl (3R)-3-[[7-(5-methyl-1,2,4-oxadiazol-3-yl)-1-isoquinolyl]amino]pyrrolidine-1-carboxylate as a yellow solid. HPLC/MS m/z: 396.2035 [M+H]⁺, Rt (U): 2.39 min.

Example 225.2: To a solution of tert-butyl (3R)-3-[[7-(5-methyl-1,2,4-oxadiazol-3-yl)-1-isoquinolyl]amino]pyrrolidine-1-carboxylate (104 mg, 0.263 mmol) in anhydrous DCM (1.3 mL) at rt under an argon atmosphere was added trifluoroacetic acid (0.20 mL, 2.6299 mmol). The resulting solution was stirred at rt overnight. Volatiles were removed under reduced pressure. The crude material was dissolved in MeOH and filtered through a 2 g SCX-2 cartridge. The product was released with 2 M ammonia in MeOH to yield 76 mg (98%) of 7-(5-methyl-1,2,4-oxadiazol-3-yl)-N-[(3R)-pyrrolidin-3-yl]isoquinolin-1-amine as an amorphous, off-white solid. HPLC/MS m/z: 296.1508 [M+H]⁺, Rt (X): 1.22 min.

Example 225.3: tert-Butyl 2-amino-4-methyl-thiazole-5-carboxylate [Example 110.1](51 mg, 0.237 mmol) and 1,1′-carbonyldiimidazole (42 mg, 0.261 mmol) were dissolved in a mixture of anhydrous DCM (1.80 mL) and anhydrous DMF (0.30 mL) under an argon atmosphere and cooled in an ice bath. The stirred reaction mixture was then heated at 40° C. for 2 h. The reaction was cooled to rt and 7-(5-methyl-1,2,4-oxadiazol-3-yl)-N-[(3R)-pyrrolidin-3-yl]isoquinolin-1-amine (70 mg, 0.237 mmol) was added in one portion. The reaction mixture was again heated at 40° C. for 10 min. The reaction mixture was concentrated under reduced pressure and purified by reverse flash chromatography (20-80% MeOH in water [0.1% FA]). Fractions containing product were combined and filtered through a 1 g SCX-2 cartidge. The product was released with 2 M ammonia in MeOH to yield 90 mg (70%) of tert-butyl 4-methyl-2-[[(3R)-3-[[7-(5-methyl-1,2,4-oxadiazol-3-yl)-1-isoquinolyl]amino]pyrrolidine-1-carbonyl]amino]thiazole-5-carboxylate as an amorphous, off-white solid. HPLC/MS m/z: 536.2083 [M+H]⁺, Rt (X): 2.65 min. ¹H NMR (600 MHz, DMSO-d₆) δ 11.07 (s, 1H), 9.01 (s, 1H), 8.16 (dd, J=8.5, 1.6 Hz, 1H), 8.01 (d, J=5.7 Hz, 1H), 7.89-7.82 (m, 2H), 7.02 (d, J=5.7 Hz, 1H), 4.89-4.63 (m, 1H), 3.98-3.75 (m, 1H), 3.75-3.46 (m, 3H), 2.70 (s, 3H), 2.48 (s, 3H), 2.32-2.09 (m, 2H), 1.49 (s, 9H).

Example 226: tert-Butyl 4-methyl-2-[[(3S)-3-[[7-(5-methyl-1,2,4-oxadiazol-3-yl)-1-isoquinolyl]amino]pyrrolidine-1-carbonyl]amino]thiazole-5-carboxylate

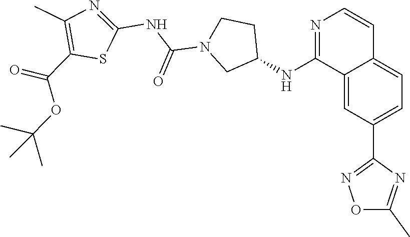 [SMILES: Cc1nc(-c2ccc3ccnc(N[C@H]4CCN(C(=O)Nc5nc(C)c(C(=O)OC(C)(C)C)s5)C4)c3c2)no1]

Example 226.1: Preparation as described for intermediate in Example 224.1, using 5-methyl-3-(2-oxidoisoquinolin-2-ium-7-yl)-1,2,4-oxadiazole [Example 96](187 mg, 0.823 mmol) and (S)-(+)-1-Boc-3-aminopyrrolidine (0.17 mL, 1.029 mmol). Purification by flash chromatography to afford tert-butyl (3S)-3-[[7-(5-methyl-1,2,4-oxadiazol-3-yl)-1-isoquinolyl]amino]pyrrolidine-1-carboxylate. Yield: 180 mg (55%) yellow solid. HPLC/MS m/z: 396.2039 [M+H]⁺, Rt (X): 2.37 min.

Example 226.2: Preparation as described for intermediate in Example 224.2, using tert-butyl (3S)-3-[[7-(5-methyl-1,2,4-oxadiazol-3-yl)-1-isoquinolyl]amino]pyrrolidine-1-carboxylate (106 mg, 0.268 mmol). Purification by SCX-2 filtration to afford 7-(5-methyl-1,2,4-oxadiazol-3-yl)-N-[(3S)-pyrrolidin-3-yl]isoquinolin-1-amine. Yield: 78 mg (99%) amorphous, off-white solid. HPLC/MS m/z: 296.1511 [M+H]⁺, Rt (X): 1.19 min.

Example 226.3: Preparation as described for Example 224.3 using tert-butyl 2-amino-4-methyl-thiazole-5-carboxylate [Example 110.1](51 mg, 0.237 mmol) and 7-(5-methyl-1,2,4-oxadiazol-3-yl)-N-[(3S)-pyrrolidin-3-yl]isoquinolin-1-amine (70 mg, 0.237 mmol). Purification by reverse flash chromatography followed by SCX-2 filtration to afford tert-butyl 4-methyl-2-[[(3S)-3-[[7-(5-methyl-1,2,4-oxadiazol-3-yl)-1-isoquinolyl]amino]pyrrolidine-1-carbonyl]amino]thiazole-5-carboxlate. Yield: 93 mg (73%) amorphous, off-white solid. HPLC/MS m/z: 536.2085 [M+H]⁺, Rt (X): 2.65 min. ¹H NMR (600 MHz, DMSO-d₆) δ 11.07 (s, 1H), 9.01 (s, 1H), 8.16 (dd, J=8.5, 1.5 Hz, 1H), 8.01 (d, J=5.7 Hz, 1H), 7.89-7.81 (m, 2H), 7.02 (d, J=5.7 Hz, 1H), 4.86-4.65 (m, 1H), 3.96-3.74 (m, 1H), 3.74-3.47 (m, 3H), 2.70 (s, 3H), 2.48 (s, 3H), 2.32-2.08 (m, 2H), 1.49 (s, 9H).

### Example 227: tert-Butyl 4-methyl-2-[methyl-[(3S)-3-[[7-(5-methyl-1,2,4-oxadiazol-3-yl)-1-isoquinolyl]amino]pyrrolidine-1-carbonyl]amino]thiazole-5-carboxylate

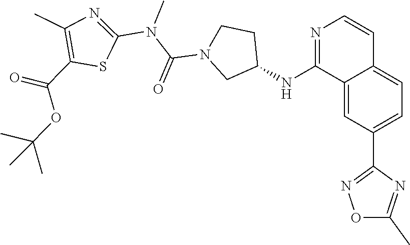 [SMILES: Cc1nc(-c2ccc3ccnc(N[C@H]4CCN(C(=O)N(C)c5nc(C)c(C(=O)OC(C)(C)C)s5)C4)c3c2)no1]

tert-Butyl 4-methyl-2-[[(3S)-3-[[7-(5-methyl-1,2,4-oxadiazol-3-yl)-1-isoquinolyl]amino]pyrrolidine-1-carbonyl]amino]thiazole-5-carboxylate (50 mg, 0.093 mmol) was dissolved in anhydrous DMF (0.37 mL) under an argon atmosphere and cooled in an ice bath. Iodomethane (6.4 uL, 0.103 mmol) and sodium hydride (3.9 mg, 0.098 mmol) were added successively. The reaction mixture was allowed to warm to ambient temperature and stirred for 30 min. The reaction mixture was directly purified by flash chromatography (20-60% EtOAc in cyclohexane) followed by reverse flash chromatography (40-100% MeOH in water [0.1% FA]) to yield 31 mg (60%) of tert-butyl 4-methyl-2-[methyl-[(3S)-3-[[7-(5-methyl-1,2,4-oxadiazol-3-yl)-1-isoquinolyl]amino]pyrrolidine-1-carbonyl]amino]thiazole-5-carboxylate as an off-white solid. HPLC/MS m/z: 550.2230 [M+H]⁺, Rt (Z): 2.76 min. 1 H NMR (600 MHz, DMSO-d₆) δ 9.02 (d, J=1.6 Hz, 1H), 8.15 (dd, J=8.4, 1.5 Hz, 1H), 8.01 (d, J=5.7 Hz, 1H), 7.88-7.83 (m, 2H), 7.01 (dd, J=5.8, 2.1 Hz, 1H), 4.80-4.70*(m, 1H), 4.00-3.94 (m, 1H)*, 3.81-3.75*(m, 2H), 3.66-3.59*(m, 2H), 3.58-3.50*(m, 4H), 3.49-3.42*(m, 2H), 2.70 (s, 3H), 2.58*(s, 3H), 2.55*(s, 3H), 2.30-2.23*(m, 1H), 2.14-2.06*(m, 1H), 1.49*(s, 9H), 1.49*(s, 9H). Note: signal splitting observed due to rotamer formation. Rotamer signals are marked with * and integral are given as their respective full signal.

### Example 228: tert-Butyl 4-methyl-2-[[3-[[3-(5-methyl-1,2,4-oxadiazol-3-yl)benzoyl]amino]azetidine-1-carbonyl]amino]thiazole-5-carboxylate

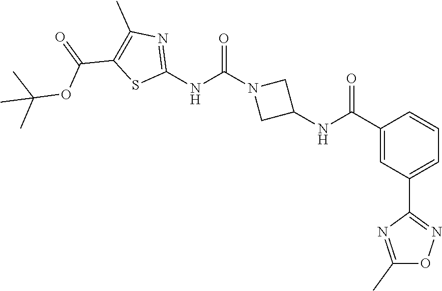 [SMILES: Cc1nc(-c2cccc(C(=O)NC3CN(C(=O)Nc4nc(C)c(C(=O)OC(C)(C)C)s4)C3)c2)no1]

Example 228.1: tert-Butyl 2-amino-4-methyl-thiazole-5-carboxylate [Example 110.1](500 mg, 2.33 mmol) and 1,1′-carbonyldiimidazole (416 mg, 2.57 mmol) were dissolved in a mixture of anhydrous DCM and anhydrous DMF (6:1, 20 mL) at ambient temperature under an argon atmosphere. The stirred reaction mixture was then heated at 40° C. for 3 h. The reaction was cooled to rt and 3-(Boc-amino)azetidine (402 mg, 2.33 mmol) was added in one portion. The reaction mixture was again heated at 40° C. for 20 min. The reaction mixture was concentrated under reduced pressure and purified by normal phase flash chromatography (0-40% EtOAc in cyclohexane) to give 829 mg (86%) of tert-butyl 2-[[3-(tert-butoxycarbonylamino)azetidine-1-carbonyl]amino]-4-methyl-thiazole-5-carboxylate as a colorless solid. HPLC/MS (ESI): m/z: 413.1854 [M+H]⁺, Rt (Z): 2.88 min.

Example 228.2: tert-Butyl 2-[[3-(tert-butoxycarbonylamino)azetidine-1-carbonyl]amino]-4-methyl-thiazole-5-carboxylate (100 mg, 0.24 mmol) was dissolved in anhydrous 1,4-dioxane (1.00 mL) at rt under an argon atmosphere. Anhydrous tert-butanol (0.93 mL, 9.70 mmol) was added to minimize cleavage of the tert-butyl ester, followed by drop-wise addition of 4 M HCl in 1,4-dioxane (1.82 mL, 7.27 mmol). The reaction mixture was continued to stir for 1 h. Volatiles were removed under reduced pressure to yield 85 mg (100%) of tert-butyl 2-[(3-aminoazetidine-1-carbonyl)amino]-4-methyl-thiazole-5-carboxylate hydrochloride, which was used in the next reaction without further purification. HPLC/MS m/z: 313.1328 [M+H]⁺, Rt (Z): 1.92 min.

Example 228.3: tert-Butyl 2-[(3-aminoazetidine-1-carbonyl)amino]-4-methyl-thiazole-5-carboxylate hydrochloride (45 mg, 0.129 mmol), 3-(5-methyl-1,2,4-oxadiazol-3-yl)benzoic acid (40 mg, 0.194 mmol) and DIPEA (67 μL, 0.387 mmol) were mixed in anhydrous DMF (0.64 mL) at rt under an argon atmosphere. 50% T₃P in DMF (0.11 mL, 0.194 mmol) was added and the reaction mixture was stirred at rt for 1 h. The reaction mixture was mixed with EtOAc (50 mL) and washed with saturated NaHCO₃ (2×30 mL) and saturated NaCl (30 mL), dried over anhydrous MgSO₄ and concentrated under reduced pressure. The crude material was purified by reverse phase flash chromatography (20-80% MeOH in water, 0.1% formic acid) to yield 33 mg (51%) of tert-butyl 4-methyl-2-[[3-[[3-(5-methyl-1,2,4-oxadiazol-3-yl)benzoyl]amino]azetidine-1-carbonyl]amino]thiazole-5-carboxylate as a colorless solid. HPLC/MS m/z: 499.1762 [M+H]⁺, Rt (Z): 2.78 min. ¹H NMR (600 MHz, DMSO-d₆) δ 11.44 (br s, 1H), 9.28 (d, J=7.0 Hz, 1H), 8.52 (t, J=1.8 Hz, 1H), 8.17-8.14 (m, 1H), 8.08 (dt, J=7.9, 1.5 Hz, 1H), 7.68 (t, J=7.8 Hz, 1H), 4.79-4.73 (m, 1H), 4.34 (br s, 2H), 4.10-4.01 (m, 2H), 2.69 (s, 3H), 2.48 (s, 3H), 1.50 (s, 9H).

### Example 229: tert-Butyl 4-methyl-2-[methyl-[3-[[3-(5-methyl-1,2,4-oxadiazol-3-yl)benzoyl]amino]azetidine-1-carbonyl]amino]thiazole-5-carboxylate

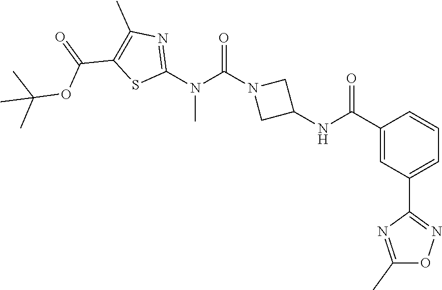 [SMILES: Cc1nc(-c2cccc(C(=O)NC3CN(C(=O)N(C)c4nc(C)c(C(=O)OC(C)(C)C)s4)C3)c2)no1]

Example 229.1: tert-Butyl 2-[[3-(tert-butoxycarbonylamino)azetidine-1-carbonyl]amino]-4-methyl-thiazole-5-carboxylate (200 mg, 0.48 mmol) and anhydrous Cs₂CO₃ (158 mg, 0.48 mmol) were mixed in anhydrous DMSO (1.00 mL) at rt under an argon atmosphere. The reaction mixture was heated at 80 ° C. Iodomethane (33 μL, 0.53 mmol) was added dropwise over 10 min. The reaction mixture was continued to stir for 1 h. The reaction mixture was cooled to ambient temperature and directly purified by normal flash chromatography (10-40% EtOAc in cyclohexane) to yield 49 mg (24%) of tert-butyl 2-[[3-(tert-butoxycarbonylamino)azetidine-1-carbonyl]-methyl-amino]-4-methyl-thiazole-5-carboxylate as a colourless, amorphous solid. HPLC/MS m/z: 427.1993 [M+H]⁺, Rt (Z): 3.15 min.

Example 229.2: Preparation as described for Example 227 using tert-butyl 2-[[3-(tert-butoxycarbonylamino)azetidine-1-carbonyl]-methyl-amino]-4-methyl-thiazole-5-carboxylate (24 mg, 0.056 mmol). Purification by SCX-2 filtration afforded tert-butyl 2-[(3-aminoazetidine-1-carbonyl)-methyl-amino]-4-methyl-thiazole-5-carboxylate. Yield 18 mg (100%) colorless solid. HPLC/MS m/z: 327.1482 [M+H]⁺, Rt (Z): 2.03 min.

Example 229.3: 3-(5-Methyl-1,2,4-oxadiazol-3-yl)benzoic acid (32 mg, 0.156 mmol), tert-butyl 2-[(3-aminoazetidine-1-carbonyl)-methyl-amino]-4-methyl-thiazole-5-carboxylate (34 mg, 0.104 mmol) and DIPEA (0.05 mL, 0.313 mmol) were mixed in anhydrous DMF (0.52 mL) at rt under an argon atmosphere. 50% T₃P in DMF (0.09 mL, 0.156 mmol) was added and the reaction mixture was stirred at rt for 1 h. The reaction mixture was mixed with EtOAc (50 mL) and washed with saturated NaCl (3×30 mL), dried over anhydrous MgSO₄ and concentrated under reduced pressure. The crude material was purified by reverse flash chromatography (40-80% MeOH in water, 0.1% formic acid) to yield 41 mg (75%) of tert-butyl 4-methyl-2-[methyl-[3-[[3-(5-methyl-1,2,4-oxadiazol-3-yl)benzoyl]amino]azetidine-1-carbonyl]amino]thiazole-5-carboxy as a colorless solid. HPLC/MS m/z: 513.1915 [M+H]⁺, Rt (Z): 2.91 min. ¹H NMR (600 MHz, DMSO-d₆) δ 9.29 (d, J=6.7 Hz, 1H), 8.52 (t, J=1.8 Hz, 1H), 8.18-8.15 (m, 1H), 8.09-8.07 (m, 1H), 7.69 (t, J=7.8 Hz, 1H), 4.79-4.72 (m, 1H), 4.48 (t, J=8.5 Hz, 2H), 4.20 (dd, J=9.0, 5.5 Hz, 2H), 3.45 (s, 3H), 2.69 (s, 3H), 2.50 (s, 3H), 1.50 (s, 9H).

### Example 230: tert-Butyl 4-methyl-2-[[3-[[7-(5-methyl-1,2,4-oxadiazol-3-yl)-1-isoquinolyl]amino]azetidine-1-carbonyl]amino]thiazole-5-carboxylate

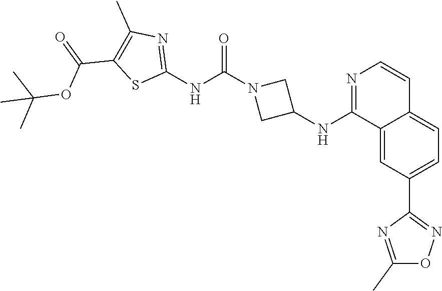 [SMILES: Cc1nc(-c2ccc3ccnc(NC4CN(C(=O)Nc5nc(C)c(C(=O)OC(C)(C)C)s5)C4)c3c2)no1]

Example 230.1: Preparation as described in Example 98, using 5-methyl-3-(2-oxidoisoquinolin-2-ium-7-yl)-1,2,4-oxadiazole [Example 96](1.08 g, 4.75 mmol) and tert-butyl 3-aminoazetidine-1-carboxylate (1.02 g, 5.94 mmol). Purification by flash chromatography followed by SCX-2 filtration to afford tert-butyl 3-[[7-(5-methyl-1,2,4-oxadiazol-3-yl)-1-isoquinolyl]amino]azetidine-1-carboxylate. Yield: 1.51 g (83%) off-white solid. HPLC/MS m/z: 382.1877 [M+H]⁺, Rt (X): 2.55 min.

Example 230.2: tert-Butyl 3-[[7-(5-methyl-1,2,4-oxadiazol-3-yl)-1-isoquinolyl]amino]azetidine-1-carboxylate (210 mg, 0.55 mmol) was dissolved in anhydrous DCM (17.5 mL) in a microwave vial at rt under an argon atmosphere. 2-Chloropyridine (0.16 mL, 1.65 mmol) and 1 M trifluoromethanesulfonic anhydride in DCM (0.83 mL, 0.83 mmol) were added successively and the reaction mixture was stirred at rt for 1 h [Note: formation and later consumption of the reactive intermediate was monitored by HPLC/MS, a methyl carbamate (m/z=340) forms upon quenching of a sample of the reaction mixture with MeOH before the analysis]. Then, DIPEA (0.58 mL, 3.30 mmol) and tert-butyl 2-amino-4-methyl-thiazole-5-carboxylate [Example 110.1](354 mg, 1.65 mmol) were added to the reaction mixture, which was continued to stir at rt overnight. Volatiles were removed under reduced pressure and the crude material was purified by normal phase flash chromatography (0-60% EtOAc in cyclohexane). Pure fractions were filtered through a 2 g SCX-2 cartridge. The product was released with 2 M ammonia in MeOH. Volatiles were evaporated to yield 68 mg (23%) of tert-butyl 4-methyl-2-[[3-[[7-(5-methyl-1,2,4-oxadiazol-3-yl)-1-isoquinolyl]amino]azetidine-1-carbonyl]amino]thiazole-5-carboxylate as a yellow solid. HPLC/MS m/z: 522.1920 [M+H]⁺, Rt (X): 2.82 min. ¹H NMR (600 MHz, DMSO-d₆) δ 11.35 (s, 1H), 9.01-8.99 (m, 1H), 8.32 (d, J=6.1 Hz, 1H), 8.18 (dd, J=8.5, 1.5 Hz, 1H), 7.98 (d, J=5.7 Hz, 1H), 7.89 (d, J=8.5 Hz, 1H), 7.07-7.05 (m, 1H), 4.93-4.87 (m, 1H), 4.40 (s, 2H), 4.14-4.07 (m, 2H), 2.71 (s, 3H), 2.48 (s, 3H), 1.50 (s, 9H).

### Example 231: tert-Butyl 4-methyl-2-[methyl-[3-[[7-(5-methyl-1,2,4-oxadiazol-3-yl)-1-isoquinolyl]amino]azetidine-1-carbonyl]amino]thiazole-5-carboxylate

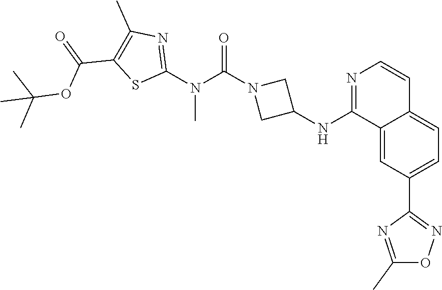 [SMILES: Cc1nc(-c2ccc3ccnc(NC4CN(C(=O)N(C)c5nc(C)c(C(=O)OC(C)(C)C)s5)C4)c3c2)no1]

tert-Butyl 4-methyl-2-[[3-[[7-(5-methyl-1,2,4-oxadiazol-3-yl)-1-isoquinolyl]amino]-azetidine-1-carbonyl]amino]thiazole-5-carboxylate (30 mg, 0.057 mmol) was dissolved in anhydrous DMF (0.28 mL) under an argon atmosphere and cooled in an ice bath. Sodium hydride 60% dispersion in mineral oil (2.4 mg, 0.060 mmol) was added and the mixture was stirred at 0° C. for 10 min. Iodomethane (4.3 uL, 0.068 mmol) was added. The reaction mixture was allowed to warm to rt and was stirred for 2 h. The reaction mixture was directly purified by normal phase flash chromatography (0-80% EtOAc in cyclohexane) followed by reverse phase flash chromatography (40-100% MeOH in water, 0.1% formic acid) to yield 5.3 mg, (17%) of tert-butyl 4-methyl-2-[methyl-[3-[[7-(5-methyl-1,2,4-oxadiazol-3-yl)-1-isoquinolyl]amino]azetidine-1-carbonyl]amino]thiazole-5-carboxylate as an off-white solid. HPLC/MS m/z: 536.2076 [M+H]⁺, Rt (X): 2.94 min.

### Example 232: N-(5-(tert-butyl)-4-methylthiazol-2-yl)-3-((7-(5-methyl-1,2,4-oxadiazol-3-yl)isoquinolin-1-yl)amino)azetidine-1-carboxamide

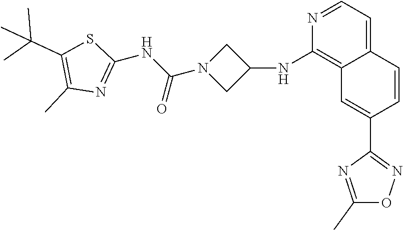 [SMILES: Cc1nc(-c2ccc3ccnc(NC4CN(C(=O)Nc5nc(C)c(C(C)(C)C)s5)C4)c3c2)no1]

Example 232.1: 5-Methyl-3-(2-oxidoisoquinolin-2-ium-7-yl)-1,2,4-oxadiazole [Example 96](111 mg, 0.4885 mmol) was dissolved/suspended in DCM (2.04 mL) and tert-butyl 3-aminoazetidine-1-carboxylate (105.16 mg, 0.6106 mmol) was added, quickly followed by DIPEA (0.26 mL, 1.4655 mmol). To this solution was added PyBroP (296.06 mg, 0.6351 mmol) and the flask stirred overnight at rt. Purification by silica gel column chromatography (eluent: 20-80% EtOAc in cyclohexane) afforded tert-butyl 3-[[7-(5-methyl-1,2,4-oxadiazol-3-yl)-1-isoquinolyl]amino]azetidine-1-carboxylate (131 mg, 70%, 0.343 mmol). HPLC/MS m/z: 382.20, [M+H]⁺, Rt (P): 1.35 min.

Example 232.2: tert-Butyl (3S)-3-[[3-(5-methyl-1,2,4-oxadiazol-3-yl)benzoyl]-amino]piperidine-1-carboxylate (116.07 mg, 0.3043 mmol) was dissolved in dry DCM (1.52 mL), trifluoroacetic acid (0.16 mL, 2.1301 mmol) was added and the solution was stirred overnight at rt. The reaction was concentrated and purified by ion exchange SCX-II column chromatography, washing with MeOH before eluting with 2M NH₃ in MeOH afforded N-(azetidin-3-yl)-7-(5-methyl-1,2,4-oxadiazol-3-yl)isoquinolin-1-amine (85 mg, 99%, 0.3022 mmol). HPLC/MS m/z: 282.14, [M+H]⁺, Rt (U): 0.85 min.

Example 232.3: N-(azetidin-3-yl)-7-(5-methyl-1,2,4-oxadiazol-3-yl)isoquinolin-1-amine (38.00 mg, 0.1351 mmol) was dissolved in DMF (0.38 mL). Triethylamine (0.02 mL, 0.1513 mmol) and N-(5-tert-butyl-4-methyl-thiazol-2-yl)imidazole-1-carboxamide (20.00 mg, 0.0757 mmol) were added and the mixture was stirred at rt for 2 h. Purification by reverse phase column chromatography (eluent: 20-80% MeOH in water (+0.1% formic acid in both)) followed by ion exchange SCX-II column chromatography, washing with MeOH before eluting with 2M NH₃ in MeOH afforded N-(5-(tert-butyl)-4-methylthiazol-2-yl)-3-((7-(5-methyl-1,2,4-oxadiazol-3-yl)isoquinolin-1-yl)amino)azetidine-1-carboxamide (23 mg, 64%, 0.0482 mmol). HPLC/MS m/z: 478.20, [M+H]⁺, Rt (Q): 2.69 min. ¹H NMR (600 MHz, DMSO-d₆) δ 10.62 (s, 1H), 9.01 (d, J=1.6 Hz, 1H), 8.31 (d, J=6.0 Hz, 1H), 8.18 (dd, J=8.5, 1.5 Hz, 1H), 7.98 (d, J=5.7 Hz, 1H), 7.89 (d, J=8.5 Hz, 1H), 7.05 (d, J=5.8 Hz, 1H), 4.92-4.81 (m, 1H), 4.34 (s, 2H), 4.04 (dd, J=9.0, 5.5 Hz, 2H), 2.71 (s, 3H), 2.27 (s, 3H), 1.34 (s, 9H).

### Example 233: Propyl 2-(3-(3-chloro-5-(5-methyl-1,2,4-oxadiazol-3-yl)benzamido)-N-methylpropanamido)-4-methylthiazole-5-carboxylate

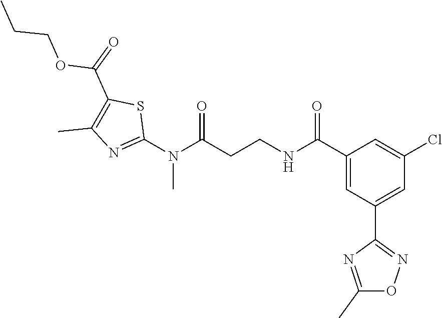 [SMILES: CCCOC(=O)c1sc(N(C)C(=O)CCNC(=O)c2cc(Cl)cc(-c3noc(C)n3)c2)nc1C]

Example 233.1: Ethyl 4-methyl-2-(methylamino)thiazole-5-carboxylate (500.00 mg, 2.4968 mmol) was dissolved at rt in THF (6.24 mL) and water (6.24 mL) and LiOH·H₂O (419.06 mg, 9.987 mmol) was added. The solution was stirred at 70° C. for 7 h. The solution was cooled down to rt and THE was removed under reduced pressure. The residue was acidified to pH=3 with citric acid solution (1 M) and filtered to afford 4-methyl-2-(methylamino)thiazole-5-carboxylic acid (350 mg, 81%, 2.0325 mmol) as a colorless solid. HPLC/MS m/z: 173.04, [M+H]⁺, Rt (P): 0.11 min.

Example 233.2: 4-Methyl-2-(methylamino)thiazole-5-carboxylic acid (345.00 mg, 2.0035 mmol) and DMAP (24.48 mg, 0.2003 mmol) were suspended in DMF (15.00 mL). 1M DCC (2.40 mL, 2.4042 mmol) and propan-1-ol (4.52 mL, 60.105 mmol) were added and the solution was stirred at 50° C. for 3.5 h. The solution was cooled down to rt and diluted with EtOAc (50 mL). The residue was washed with water (2×30 mL), brine (30 mL), dried over magnesium sulfate and concentrated under reduced pressure. Purification by silica gel column chromatography (eluent: 0-15% of EtOAc in cyclohexane) afforded propyl 4-methyl-2-(methylamino)thiazole-5-carboxylate (300 mg, 70%, 1.4 mmol) as a colorless solid. HPLC/MS m/z: 215.09, [M+H]⁺, Rt (R): 1.28 min.

Example 233.3: To a mixture of 3-[[3-chloro-5-(5-methyl-1,2,4-oxadiazol-3-yl)benzoyl]amino]propanoic acid (60.00 mg, 0.1937 mmol), propyl 4-methyl-2-(methylamino)thiazole-5-carboxylate (49.81 mg, 0.2325 mmol), EDC.HCl (74.28 mg, 0.3875 mmol) and 1-hydroxybenzotriazole (52.35 mg, 0.3875 mmol) was added under nitrogen atmosphere DMF (0.97 mL). The solution was stirred at 60° C. overnight. The solution was cooled down to rt, diluted with EtOAc (20 mL) and washed with water (20 mL), saturated aqueous bicarbonate (20 mL), brine (20 mL), dried over magnesium sulfate and concentrated in vacuo. Purified by reverse phase column chromatography (eluent: 20-100% MeOH in water) to afford propyl 2-[3-[[3-chloro-5-(5-methyl-1,2,4-oxadiazol-3-yl)benzoyl]amino]propanoyl-methyl-amino]-4-methyl-thiazole-5-carboxylate (35 mg, 36%, 0.0692 mmol) as a colorless solid. HPLC/MS m/z: 506.13, [M+H]⁺, Rt (S): 3.35 min. ¹H NMR (500 MHz, DMSO-d₆) δ 8.95 (t, J=5.4 Hz, 1H), 8.43 (t, J=1.5 Hz, 1H), 8.12-8.08 (m, 2H), 4.16 (t, J=6.5 Hz, 2H), 3.66 (s, 3H), 3.63 (td, J=6.8, 5.3 Hz, 2H), 3.08 (t, J=6.8 Hz, 2H), 2.70 (s, 3H), 2.58 (s, 3H), 1.68 (dtd, J=13.7, 7.3, 6.4 Hz, 2H), 0.95 (t, J=7.4 Hz, 3H).

### Example 234: Propyl 4-methyl-2-((2S,4S)-1-methyl-4-(3-(5-methyl-1,2,4-oxadiazol-3-yl)benzamido)pyrrolidine-2-carboxamido)thiazole-5-carboxylate

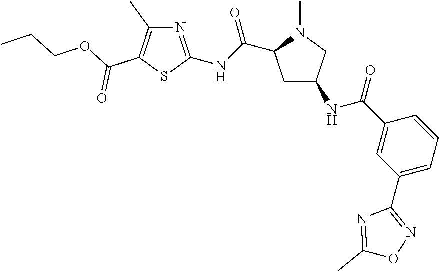 [SMILES: CCCOC(=O)c1sc(NC(=O)[C@@H]2C[C@H](NC(=O)c3cccc(-c4noc(C)n4)c3)CN2C)nc1C]

Example 234.1: To a mixture of (2S,4S)-4-(tert-butoxycarbonylamino)-1-methyl-pyrrolidine-2-carboxylic acid (50.00 mg, 0.2047 mmol), 1-hydroxybenzotriazole (55.31 mg, 0.4093 mmol), EDC.HCl (78.47 mg, 0.4093 mmol) and propyl 2-amino-4-methyl-thiazole-5-carboxylate (61.48 mg, 0.3070 mmol) was added under nitrogen atmosphere DMF (1.28 mL). The resulting solution was stirred at 45° C. for 16 h. EtOAc and water were added, the mixture was extracted with EtOAc, dried over MgSO₄ and concentrated in vacuo. Purification by silica gel column chromatography (eluent: 10-100% EtOAc in cyclohexane) to afford propyl 2-[[(2S,4S)-4-(tert-butoxycarbonylamino)-1-methyl-pyrrolidine-2-carbonyl]amino]-4-methyl-thiazole-5-carboxylate (47 mg, 54%, 0.1102 mmol). HPLC/MS m/z: 427.25, [M+H]⁺, Rt (P): 1.36 min.

Example 234.2: tert-Butyl (3S)-3-[[3-(5-methyl-1,2,4-oxadiazol-3-yl)benzoyl]-amino]piperidine-1-carboxylate (65.00 mg, 0.1524 mmol), was dissolved in dry DCM (0.76 mL), trifluoroacetic acid (0.08 mL, 1.0667 mmol) was added and the solution was stirred overnight at rt. Purification by ion exchange SCX-II column chromatography, washing with MeOH before eluting with 2M NH₃ in MeOH afforded propyl 2-[[(4S)-4-amino-1-methyl-pyrrolidine-2-carbonyl]amino]-4-methyl-thiazole-5-carboxylate (29 mg, 58%, 0.0888 mmol). HPLC/MS m/z: 327.17, [M+H]⁺, Rt (P): 1.12 min.

Example 234.3: 3-(5-Methyl-1,2,4-oxadiazol-3-yl)benzoic acid (18.14 mg, 0.0888 mmol), propyl 2-[[(2S,4S)-4-amino-1-methyl-pyrrolidine-2-carbonyl]amino]-4-methyl-thiazole-5-carboxylate (29.00 mg, 0.0888 mmol), DMF (0.44 mL), HATU (31.35 mg, 0.1333 mmol) and DIPEA (0.05 mL, 0.2665 mmol) were combined and stirred overnight at rt. Purified by reverse phase column chromatography (eluent: 20-80% MeOH in water (+0.1% formic acid in both)) to afford propyl 4-methyl-2-[[(2S,4S)-1-methyl-4-[[3-(5-methyl-1,2,4-oxadiazol-3-yl)benzoyl]amino]pyrrolidine-2-carbonyl]amino]thiazole-5-carboxylate (30 mg, 66%, 0.0585 mmol). HPLC/MS m/z: 513.19, [M+H]⁺, Rt (Q): 2.61 min. ¹H NMR (600 MHz, Chloroform-d) δ 8.41-8.38 (m, 1H), 8.17-8.12 (m, 1H), 8.01-7.96 (m, 1H), 7.94 (d, J=7.8 Hz, 1H), 7.50 (t, J=7.8 Hz, 1H), 4.80-4.73 (m, 1H), 4.21 (t, J=6.6 Hz, 2H), 3.26-3.19 (m, 2H), 2.87 (ddd, J=14.6, 10.4, 7.5 Hz, 1H), 2.78 (dd, J=10.3, 5.7 Hz, 1H), 2.67 (s, 3H), 2.55 (s, 3H), 2.43 (s, 3H), 2.08-2.02 (m, 1H), 1.77-1.69 (m, 2H), 0.98 (t, J=7.4 Hz, 3H). (Note: 1×NH not observed).

The following examples were prepared by an analogous procedure:

### Example 235: Propyl 4-methyl-2-((2R,4R)-1-methyl-4-(3-(5-methyl-1,2,4-oxadiazol-3-yl)benzamido)pyrrolidine-2-carboxamido)thiazole-5-carboxylate

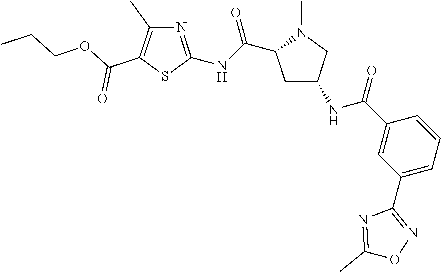 [SMILES: CCCOC(=O)c1sc(NC(=O)[C@H]2C[C@@H](NC(=O)c3cccc(-c4noc(C)n4)c3)CN2C)nc1C]

5 mg (21%). HPLC/MS m/z: 513.19, [M+H]⁺, Rt (U): 2.65 min. ¹H NMR (600 MHz, Chloroform-d) δ 8.38-8.37 (m, 1H), 8.16-8.13 (m, 1H), 7.97-7.94 (m, 1H), 7.56-7.52 (m, 1H), 7.49 (t, J=7.8 Hz, 1H), 4.78-4.72 (m, 1H), 4.21 (t, J=6.6 Hz, 2H), 3.24 (dd, J=10.4, 5.8 Hz, 2H), 2.87 (ddd, J=14.5, 10.4, 7.4 Hz, 1H), 2.81 (dd, J=10.4, 5.6 Hz, 1H), 2.66 (s, 3H), 2.57 (s, 3H), 2.45 (s, 3H), 2.10-2.04 (m, 1H), 1.78-1.70 (m, 2H), 0.99 (t, J=7.4 Hz, 3H). (Note: 1×NH not observed).

### Example 236: (2S,4S)-N-(5-(tert-butyl)-4-methylthiazol-2-yl)-1-methyl-4-((7-(5-methyl-1,2,4-oxadiazol-3-yl)isoquinolin-1-yl)amino)pyrrolidine-2-carboxamide

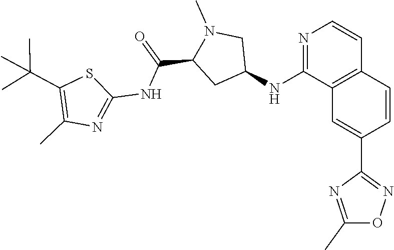 [SMILES: Cc1nc(-c2ccc3ccnc(N[C@H]4C[C@@H](C(=O)Nc5nc(C)c(C(C)(C)C)s5)N(C)C4)c3c2)no1]

### Example 236.1: Methyl (4S)-4-amino-1-methyl-L-prolinate dihydrochloride (127.15 mg, 0.5501 mmol) was dissolved in DCM (1.83 mL) and DIPEA (0.38 mL, 2.2005 mmol) and 5-methyl-3-(2-oxidoisoquinolin-2-ium-7-yl)-1,2,4-oxadiazole [Example 96](100 mg, 0.4401 mmol) was added. To this suspension was added PyBroP (266.72 mg, 0.5721 mmol) and the reaction was stirred overnight at rt. Purification by silica gel column chromatography (eluent: 10-50% EtOAc in cyclohexane) to afford methyl (2S,4S)-1-methyl-4-[[7-(5-methyl-1,2,4-oxadiazol-3-yl)-1-isoquinolyl]-amino]pyrrolidine-2-carboxylate (135 mg, 54%, 0.2388 mmol). HPLC/MS m/z: 368.17, [M+H]⁺, Rt (P): 1.10 min.

Example 236.2: Methyl (2S,4S)-1-methyl-4-[[7-(5-methyl-1,2,4-oxadiazol-3-yl)-1-isoquinolyl]amino]pyrrolidine-2-carboxylate (97.00 mg, 0.2640 mmol) was dissolved in a 1:1 mixture of MeOH and THF. Sodium hydroxide (21.65 mg, 0.5280 mmol) in water (0.278 mL, 1.9 M) was added. The solution was stirred for 1 h. The mixture was diluted with water (2 mL) and acidified with 1 M HCl (pH=2). The precipitate was filtered, washed with water (2 mL) and dried to afford (2S,4S)-1-methyl-4-[[7-(5-methyl-1,2,4-oxadiazol-3-yl)-1-isoquinolyl]amino]pyrrolidine-2-carboxylic acid (37 mg, 40%, 0.104 mmol). HPLC/MS m/z: 354.17, [M+H]⁺, Rt (P): 0.78 min.

Example 236.3: To (2S,4S)-1-methyl-4-[[7-(5-methyl-1,2,4-oxadiazol-3-yl)-1-isoquinolyl]amino]pyrrolidine-2-carboxylic acid (24.90 mg, 0.0705 mmol), HOBT (15.87 mg, 0.1175 mmol) in DMF (0.37 mL), EDC.HCl (22.52 mg, 0.1175 mmol) and 5-tert-butyl-4-methyl-thiazol-2-ylamine (10.00 mg, 0.0587 mmol) were added under nitrogen atmosphere. The resulting solution was stirred at 45° C. overnight. Purified by reverse phase column chromatography (eluent: 30-80% MeOH in water (+0.1% formic acid in both)) to afford (2S,4S)-N-(5-tert-butyl-4-methyl-thiazol-2-yl)-1-methyl-4-[[7-(5-methyl-1,2,4-oxadiazol-3-yl)-1-isoquinolyl]amino]pyrrolidine-2-carboxamide (1 mg, 3%, 0.0020 mmol). HPLC/MS m/z: 506.23, [M+H]⁺, Rt (Q): 2.70 min. ¹H NMR (600 MHz, Chloroform-d) δ 8.72 (s, 1H), 8.52 (q, J=21.0, 17.9 Hz, 1H), 8.20 (dd, J=8.5, 1.5 Hz, 1H), 8.00 (d, J=5.8 Hz, 1H), 7.71 (d, J=8.5 Hz, 1H), 6.92 (d, J=5.8 Hz, 1H), 6.85 (s, 1H), 4.88 (d, J=6.8 Hz, 1H), 3.34 (d, J=10.3 Hz, 1H), 3.22 (dd, J=10.1, 6.3 Hz, 1H), 2.93 (ddd, J=14.3, 10.2, 7.3 Hz, 1H), 2.87 (dd, J=10.3, 5.8 Hz, 1H), 2.71 (s, 3H), 2.46 (s, 3H), 2.27 (s, 3H), 2.18-2.12 (m, 1H), 1.35 (s, 9H).

The following examples were prepared by an analogous procedure:

### Example 237: Propyl 4-methyl-2-((2S,4S)-1-methyl-4-((7-(5-methyl-1,2,4-oxadiazol-3-yl)isoquinolin-1-yl)amino)pyrrolidine-2-carboxamido)thiazole-5-carboxylate

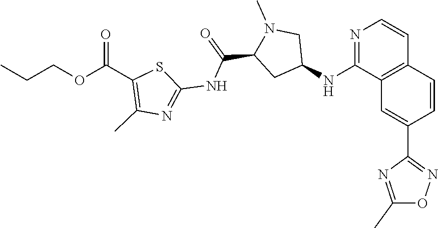 [SMILES: CCCOC(=O)c1sc(NC(=O)[C@@H]2C[C@H](Nc3nccc4ccc(-c5noc(C)n5)cc34)CN2C)nc1C]

6.5 mg (9%). HPLC/MS m/z: 536.21, [M+H]⁺, Rt (T): 1.33 min. ¹H NMR (600 MHz, Chloroform-d) δ 8.44-8.42 (m, 1H), 8.18 (dd, J=8.4, 1.5 Hz, 1H), 8.03 (d, J=5.8 Hz, 1H), 7.72 (d, J=8.5 Hz, 1H), 6.97 (dd, J=5.9, 0.9 Hz, 1H), 5.73 (d, J=5.6 Hz, 1H), 4.81-4.75 (m, 1H), 4.21-4.17 (m, 2H), 3.43-3.39 (m, 1H), 3.35 (dd, J=10.4, 5.2 Hz, 1H), 2.95-2.86 (m, 2H), 2.66 (s, 3H), 2.53 (s, 3H), 2.45 (s, 3H), 2.23-2.17 (m, 1H), 1.76-1.69 (m, 2H), 0.98 (t, J=7.4 Hz, 3H).

### Example 238: (2S,4S)-1-methyl-4-((7-(5-methyl-1,2,4-oxadiazol-3-yl)isoquinolin-1-yl)amino)-N-(1-methyl-5-pentyl-1 H-pyrazol-3-yl)pyrrolidine-2-carboxamide

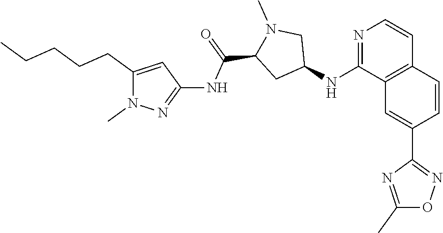 [SMILES: CCCCCc1cc(NC(=O)[C@@H]2C[C@H](Nc3nccc4ccc(-c5noc(C)n5)cc34)CN2C)nn1C]

12 mg (23%). HPLC/MS m/z: 503.29, [M+H]⁺, Rt (U): 2.93 min. ¹H NMR (600 MHz, Chloroform-d) δ 9.92 (s, 1H), 8.63 (s, 1H), 8.19 (dd, J=8.4, 1.5 Hz, 1H), 8.01 (d, J=5.8 Hz, 1H), 7.71 (d, J=8.5 Hz, 1H), 6.92 (d, J=5.8 Hz, 1H), 6.49 (s, 1H), 4.83 (d, J=7.9 Hz, 1H), 3.57 (s, 3H), 3.31 (dd, J=10.2, 1.5 Hz, 1H), 3.14 (dd, J=10.0, 6.5 Hz, 1H), 2.96-2.79 (m, 2H), 2.69 (s, 3H), 2.47 (d, J=6.8 Hz, 5H), 2.19-2.04 (m, 1H), 1.67-1.50 (m, 2H), 1.34-1.25 (m, 4H), 0.92-0.84 (m, 3H). (Note: 1×NH not observed).

### Example 239: Propyl 4-methyl-2-((2S,4S)-4-((7-(5-methyl-1,2,4-oxadiazol-3-yl)isoquinolin-1-yl)amino)pyrrolidine-2-carboxamido)thiazole-5-carboxylate

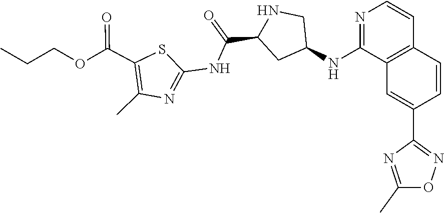 [SMILES: CCCOC(=O)c1sc(NC(=O)[C@@H]2C[C@H](Nc3nccc4ccc(-c5noc(C)n5)cc34)CN2)nc1C]

Methyl (2S,4S)-1-Boc-4-aminopyrrolidine-2-carboxylate hydrochloride was used to prepare propyl 2-[[(2S,4S)-1-tert-butoxycarbonyl-4-[[7-(5-methyl-1,2,4-oxadiazol-3-yl)-1-isoquinolyl]amino]pyrrolidine-2-carbonyl]amino]-4-methyl-thiazole-5-carboxylate using a procedure analogous to Example 235. To a mixture of propyl 2-[[(2S,4S)-1-tert-butoxycarbonyl-4-[[7-(5-methyl-1,2,4-oxadiazol-3-yl)-1-isoquinolyl]amino]pyrrolidine-2-carbonyl]amino]-4-methyl-thiazole-5-carboxylate (22.00 mg, 0.0354 mmol) in DCM (0.18 mL) was added trifluoroacetic acid (0.02 mL, 0.2477 mmol). The resulting solution was stirred at rt for 2 h. Concentrated in vacuo and purified by reverse phase column chromatography (eluent: 20-80% MeOH in water (+0.1% formic acid in both). Further purication by silica gel column chromatography (eluent: 2-5% MeOH in DCM) and then preparative TLC (DCM/MeOH: 96/4, 4 elutions) afforded propyl 4-methyl-2-((2S,4S)-4-((7-(5-methyl-1,2,4-oxadiazol-3-yl)isoquinolin-1-yl)amino)pyrrolidine-2-carboxamido)thiazole-5-carboxylate (2 mg, 10%, 0.0035 mmol). HPLC/MS m/z: 522.19 [M+H]⁺, Rt (S): 2.63 min. ¹H NMR (600 MHz, Chloroform-d) δ 11.05 (s, 1H), 8.32 (s, 1H), 8.18 (dd, J=8.5, 1.5 Hz, 1H), 8.04 (d, J=5.8 Hz, 1H), 7.74 (d, J=8.5 Hz, 1H), 6.99 (d, J=5.8 Hz, 1H), 5.42 (s, 1H), 4.76 (q, J=5.9 Hz, 1H), 4.19 (td, J=6.6, 2.4 Hz, 2H), 4.15 (dd, J=9.4, 5.3 Hz, 1H), 3.65 (dd, J=11.3, 6.0 Hz, 1H), 3.03 (dd, J=11.3, 5.5 Hz, 1H), 2.81 (ddd, J=13.6, 9.5, 6.7 Hz, 1H), 2.67 (s, 3H), 2.56 (s, 3H), 2.30 (dt, J=13.4, 5.6 Hz, 1H), 1.73 (sext, J=7.1 Hz, 2H), 0.99 (t, J=7.4 Hz, 3H). (Note: 1×NH not observed).

### Example 240: Propyl (S)-4-methyl-2-(3-(3-(5-methyl-1,2,4-oxadiazol-3-yl)benzamido)pyrrolidine-1-carbonyl)thiazole-5-carboxylate

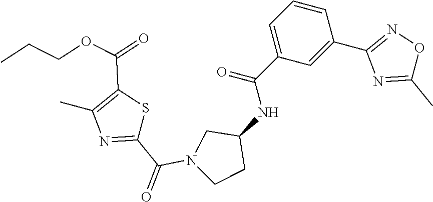 [SMILES: CCCOC(=O)c1sc(C(=O)N2CC[C@H](NC(=O)c3cccc(-c4noc(C)n4)c3)C2)nc1C]

Example 240.1: 3-(5-methyl-1,2,4-oxadiazol-3-yl)benzoic acid (152.92 mg, 0.7490 mmol), (S)-(−)-1-Boc-3-aminopyrrolidine (139.49 mg, 0.7490 mmol), DMF (3.75 mL), HATU (264.31 mg, 1.1234 mmol) and DIPEA (0.39 mL, 2.2469 mmol) were combined and stirred overnight at rt. Water (150 mL) was added and the mixture extracted with EtOAc (3×50 mL). The organics were combined, washed with saturated NaHCO₃ (50 mL), brine (50 mL), dried over MgSO₄. Purification by silica gel column chromatography (eluent: EtOAc in cychexane 50-100%) to afford tert-butyl (3S)-3-[[3-(5-methyl-1,2,4-oxadiazol-3-yl)benzoyl]amino]pyrrolidine-1-carboxylate (189 mg, 68%, 0.5075 mmol). HPLC/MS m/z: 395.17, [M+Na]⁺, Rt (Q): 1.46 min.

Example 240.2: tert-Butyl (3S)-3-[[3-(5-methyl-1,2,4-oxadiazol-3-yl)benzoyl]amino]piperidine-1-carboxylate (184.00 mg, 0.4941 mmol) was dissolved in dry DCM (2.54 mL), trifluoroacetic acid (0.26 mL, 3.4585 mmol) was added to the solution and stirred 2 h at rt. The reaction was concentrated and purified by ion exchange SCX-II column chromatography, washing with MeOH before eluting with 2M NH₃ in MeOH to afford 3-(5-methyl-1,2,4-oxadiazol-3-yl)-N-[(3S)-pyrrolidin-3-yl]benzamide (113 mg, 84%, 0.4150 mmol). HPLC/MS m/z: 273.13, [M+H]⁺, Rt (P): 0.80 min.

Example 240.3: To a stirring solution containing 4-methyl-5-propoxycarbonyl-thiazole-2-carboxylic acid [Example 74.5](34.09 mg, 0.0595 mmol), 3-(5-methyl-1,2,4-oxadiazol-3-yl)-N-[(3S)-pyrrolidin-3-yl]benzamide (27.00 mg, 0.0992 mmol) and HATU (56.00 mg, 0.1473 mmol) in DMF (0.59 mL) was added DIPEA (0.03 mL, 0.1981 mmol). The resulting solution was stirred at rt overnight. The solution was added to water (15 mL) and the residue was extracted with EtOAc (3×10 mL). The combined organic layer was washed with brine (15 mL) and concentrated under reduced pressure. Purified by reverse phase column chromatography (eluent: 30-100% MeOH in water (+0.1% formic acid in both)) to afford propyl (S)-4-methyl-2-(3-(3-(5-methyl-1,2,4-oxadiazol-3-yl)benzamido)pyrrolidine-1-carbonyl)thiazole-5-carboxylate (13 mg, 45%, 0.0269 mmol) as a colorless solid. HPLC/MS m/z: 484.16, [M+H]⁺, Rt (Q): 3.02 min. ¹H NMR (500 MHz, DMSO-d₆) δ 8.88 (t, J=5.6 Hz, 1H), 8.50-8.45 (m, 1H), 8.13 (d, J=7.7 Hz, 1H), 8.08-8.03 (m, 1H), 7.66 (t, J=7.8 Hz, 1H), 4.67-4.50 (m, 1H), 4.33-4.03 (m, 4H), 3.88-3.70 (m, 1H), 3.69-3.62 (m, 1H), 2.70-2.67 (m, 6H), 2.31-1.97 (m, 2H), 1.76-1.65 (m, 2H), 1.01-0.90 (m, 3H). (Note: Two rotameric forms present)

The following examples were prepared in an analogous procedure:

### Example 241: Propyl (R)-4-methyl-2-(3-(3-(5-methyl-1,2,4-oxadiazol-3-yl)benzamido)pyrrolidine-1-carbonyl)thiazole-5-carboxylate

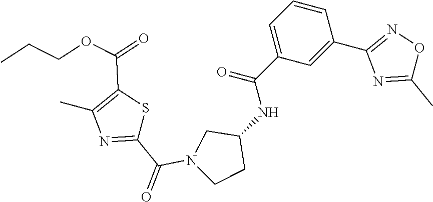 [SMILES: CCCOC(=O)c1sc(C(=O)N2CC[C@@H](NC(=O)c3cccc(-c4noc(C)n4)c3)C2)nc1C]

15 mg (52%), as a colorless solid. HPLC/MS m/z: 485.17, [M+H]⁺, Rt (Q): 3.02 min. ¹H NMR (500 MHz, DMSO-d₆) δ 8.91-8.85 (m, 1H), 8.47 (dt, J=8.4, 1.9 Hz, 1H), 8.13 (d, J=7.7 Hz, 1H), 8.10-8.03 (m, 1H), 7.70-7.62 (m, 1H), 4.67-4.52 (m, 1H), 4.32-4.03 (m, 4H), 3.87-3.61 (m, 2H), 2.72-2.66 (m, 6H), 2.31-1.97 (m, 2H), 1.77-1.62 (m, 2H), 1.00-0.89 (m, 3H). (Note: Two rotameric forms present)

### Example 242: Propyl 4-methyl-2-[(3S)-3-[[3-(2-methyltetrazol-5-yl)benzoyl]amino]pyrrolidine-1-carbonyl]thiazole-5-carboxylate

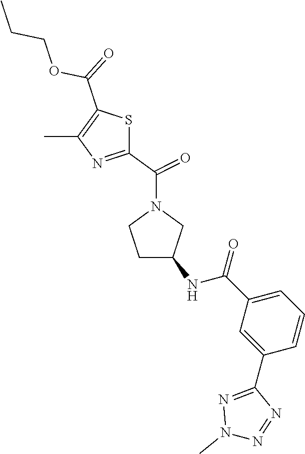 [SMILES: CCCOC(=O)c1sc(C(=O)N2CC[C@H](NC(=O)c3cccc(-c4nnn(C)n4)c3)C2)nc1C]

Example 242.1: Procedure from Example 239 and 3-(2-Methyl-2H-tetrazol-5-yl)-benzoic acid afforded 3-(2-methyltetrazol-5-yl)-N-[(3S)-pyrrolidin-3-yl]benzamide. HPLC/MS m/z: 273.15, [M+H]⁺, Rt (T): 0.68 min.

Example 242.2: To a stirring solution containing 4-methyl-5-propoxycarbonyl-thiazole-2-carboxylic acid [Example 74.5](38.27 mg, 0.0551 mmol), 3-(2-methyltetrazol-5-yl)-N-[(3S)-pyrrolidin-3-yl]benzamide (15.00 mg, 0.0551 mmol) and HATU (31.42 mg, 0.0826 mmol) in DMF (0.40 mL) was added DIPEA (0.03 mL, 0.1653 mmol). The resulting solution was stirred at rt for 6 d. Purified by reverse phase column chromatography (eluent: 30-100% MeOH/H₂O+0.1% formic acid) followed by ion exchange SCX-II column chromatography, washing with MeOH before eluting with 2M NH₃ in MeOH to give propyl 4-methyl-2-[(3S)-3-[[3-(2-methyltetrazol-5-yl)benzoyl]amino]pyrrolidine-1-carbonyl]thiazole-5-carboxylate (7.7 mg, 29%, 0.0159 mmol) as a light beige, amorphous solid. HPLC/MS m/z: 484.18 [M+H]⁺, Rt (T): 1.44 min. ¹H NMR (600 MHz, DMSO-d₆) δ 8.87 (t, J=6.1 Hz, 1H), 8.54 (dt, J=9.9, 1.8 Hz, 2H), 8.20 (dt, J=7.8, 1.4 Hz, 2H), 8.02 (tt, J=7.5, 1.5 Hz, 2H), 7.66 (td, J=7.8, 1.6 Hz, 2H), 4.63 (quint, J=6.5, 6.1 Hz, 1H), 4.56 (q, J=5.7, 5.3 Hz, 1H), 4.45 (s, 6H), 4.29 (dd, J=12.4, 6.4 Hz, 1H), 4.23 (td, J=6.5, 3.3 Hz, 4H), 4.21-4.14 (m, 1H), 4.07 (dd, J=12.4, 4.5 Hz, 1H), 3.84 (dd, J=13.0, 6.5 Hz, 1H), 3.74 (dt, J=12.5, 7.5 Hz, 1H), 3.66 (ddd, J=12.4, 7.1, 5.1 Hz, 2H), 2.70 (d, J=5.2 Hz, 6H), 2.27 (dtd, J=13.7, 7.9, 5.9 Hz, 1H), 2.22-2.14 (m, 1H), 2.11 (dd, J=12.2, 6.5 Hz, 1H), 2.03 (dq, J=12.7, 6.0 Hz, 1H), 1.70 (qd, J=6.8, 3.1 Hz, 4H), 0.95 (td, J=7.4, 2.9 Hz, 6H). (Note: Presence of rotamers).

### Example 243: Propyl (S)-4-methyl-2-(3-((7-(5-methyl-1,2,4-oxadiazol-3-yl)isoquinolin-1-yl)amino)pyrrolidine-1-carbonyl)thiazole-5-carboxylate

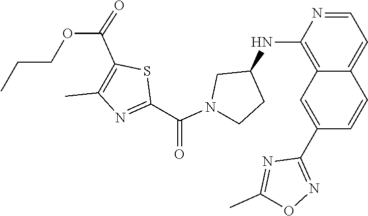 [SMILES: CCCOC(=O)c1sc(C(=O)N2CC[C@H](Nc3nccc4ccc(-c5noc(C)n5)cc34)C2)nc1C]

Example 243.1: 5-Methyl-3-(2-oxidoisoquinolin-2-ium-7-yl)-1,2,4-oxadiazole [Example 96](50 mg, 0.2201 mmol) was dissolved in DCM (0.92 mL) and (S)-(−)-1-Boc-3-aminopyrrolidine (51.23 mg, 0.2751 mmol) was added, quickly followed by DIPEA (0.14 mL, 0.8252 mmol). PyBroP (133.36 mg, 0.2861 mmol) was added, and the mixture was stirred overnight at rt. Purification by silica gel column chromatography (eluent: 10-50% EtOAc in cyclohexane) to afford tert-butyl (3S)-3-[[7-(5-methyl-1,2,4-oxadiazol-3-yl)-1-isoquinolyl]amino]pyrrolidine-1-carboxylate (39 mg, 45%, 0.0986 mmol). HPLC/MS m/z: 396.20, [M+H]⁺, Rt (T): 1.16 min.

Example 243.2: tert-Butyl (3S)-3-[[3-(5-methyl-1,2,4-oxadiazol-3-yl)benzoyl]-amino]piperidine-1-carboxylate (39.00 mg, 0.0986 mmol) was dissolved in dry DCM (0.49 mL), trifluoroacetic acid (0.05 mL, 0.6904 mmol) was added and the solution was stirred at rt overnight. Purification by ion exchange SCX-II column chromatography, washing with MeOH before eluting with 2M NH₃ in MeOH afforded 7-(5-methyl-1,2,4-oxadiazol-3-yl)-N-[(3S)-pyrrolidin-3-yl]isoquinolin-1-amine (25 mg, 86%, 0.0846 mmol). HPLC/MS m/z: 296.15, [M+H]⁺, Rt (T): 0.67 min.

Example 243.3: To a stirring solution containing 4-methyl-5-propoxycarbonyl-thiazole-2-carboxylic acid (23.99 mg, 0.1047 mmol), 7-(5-methyl-1,2,4-oxadiazol-3-yl)-N-[(3S)-pyrrolidin-3-yl]isoquinolin-1-amine (34.00 mg, 0.1151 mmol) and HATU (74.41 mg, 0.1957 mmol) in DMF (0.58 mL) was added DIPEA (0.06 mL, 0.3454 mmol). The resulting solution was stirred at rt overnight. Purified by reverse phase column chromatography (eluent: 15-80% MeOH in water (+0.1% formic acid in both)) to afford propyl 4-methyl-2-[(3S)-3-[[7-(5-methyl-1,2,4-oxadiazol-3-yl)-1-isoquinolyl]amino]pyrrolidine-1-carbonyl]thiazole-5-carboxylate (9 mg, 15%, 0.0178 mmol) as a yellow solid. HPLC/MS m/z: 507.18, [M+H]⁺, Rt (T): 1.38 min. ¹H NMR (600 MHz, Chloroform-d) δ 8.55 (s, 1H), 8.23 (ddd, J=8.6, 3.3, 1.5 Hz, 1H), 8.07 (d, J=5.8 Hz, 1H), 7.77 (d, J=8.5 Hz, 1H), 7.01 (d, J=5.8 Hz, 1H), 5.83-5.70 (m, 1H), 5.05-4.87 (m, 1H), 4.62-4.54 (m, 0.6H), 4.41-4.14 (m, 4H), 3.99-3.83 (m, 1.4H), 2.76-2.70 (m, 3H), 2.67 (s, 3H), 2.53-2.12 (m, 2H), 1.81-1.70 (m, 2H), 1.06-0.95 (m, 3H). (Note: Two rotameric forms present).

The following examples were prepared in an analogous procedure:

### Example 244: Propyl (R)-4-methyl-2-(3-((7-(5-methyl-1,2,4-oxadiazol-3-yl)isoquinolin-1-yl)amino)pyrrolidine-1-carbonyl)thiazole-5-carboxylate

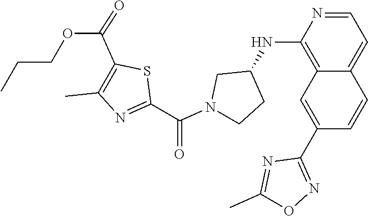 [SMILES: CCCOC(=O)c1sc(C(=O)N2CC[C@@H](Nc3nccc4ccc(-c5noc(C)n5)cc34)C2)nc1C]

2.3 mg (11%), colorless solid. HPLC/MS m/z: 507.18, [M+H]⁺, Rt (T): 1.38 min. ¹H NMR (600 MHz, Chloroform-d) δ 8.51 (s, 1H), 8.26 (d, J=8.5 Hz, 1H), 8.12-8.05 (m, 1H), 7.79 (d, J=8.5 Hz, 1H), 7.02 (d, J=5.8 Hz, 1H), 5.58 (s, 1H), 5.10-4.86 (m, 1H), 4.65-4.56 (m, 0.6H), 4.43-4.13 (m, 4H), 4.02-3.72 (m, 1.4H), 2.78-2.72 (m, 3H), 2.71-2.67 (m, 3H), 2.55-2.11 (m, 2H), 1.82-1.72 (m, 2H), 1.05-0.98 (m, 3H). (Note: Two rotameric forms present).

### Example 245: Propyl (S)-1-methyl-3-(3-((7-(5-methyl-1,2,4-oxadiazol-3-yl)isoquinolin-1-yl)amino)pyrrolidine-1-carbonyl)-1 H-pyrazole-5-carboxylate

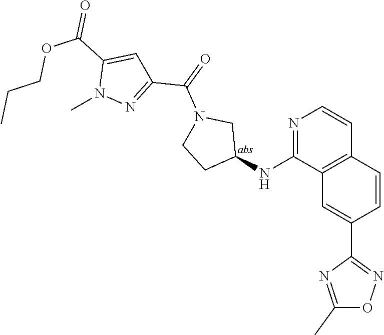 [SMILES: CCCOC(=O)c1cc(C(=O)N2CC[C@H](Nc3nccc4ccc(-c5noc(C)n5)cc34)C2)nn1C]

Example 245.1: tert-Butyl (3S)-3-[[3-(5-methyl-1,2,4-oxadiazol-3-yl)benzoyl]amino]-pyrrolidine-1-carboxylate (42 mg, 51%, 0.0883 mmol) was prepared using the procedure for Example 242. HPLC/MS m/z: 476.20, [M+H]⁺, Rt (T): 1.06 min.

Example 245.2: tert-Butyl (3S)-3-[[3-(5-methyl-1,2,4-oxadiazol-3-yl)benzoyl]amino]-pyrrolidine-1-carboxylate (37.00 mg, 0.0778 mmol) was dissolved at rt in THF (0.41 mL) and water (0.41 mL) and LiOH hydrate (13.06 mg, 0.3113 mmol) was added. The resulting solution was heated at 70° C. for 2 h. The solution was cooled down to rt and volatiles were removed under reduced pressure. Water (2 mL) was added, and the solution was then acidified by adding few drops of HCl 37% until a pH around 1 was reached. Solid crashed out in the solution, filtered off, washed with water (5 mL) and dried under high vacuum to afford 2-methyl-5-[(3S)-3-[[7-(5-methyl-1,2,4-oxadiazol-3-yl)-1-isoquinolyl]amino]pyrrolidine-1-carbonyl]pyrazole-3-carboxylic acid (34 mg, 98%, 0.0760 mmol) as a colorless solid. HPLC/MS m/z: 448.17, [M+H]⁺, Rt (T): 0.91 min.

Example 245.3: 2-Methyl-5-[(3S)-3-[[7-(5-methyl-1,2,4-oxadiazol-3-yl)-1-isoquinolyl]amino]pyrrolidine-1-carbonyl]pyrazole-3-carboxylic acid (55.00 mg, 0.1229 mmol) and 1-propanol (0.09 mL, 1.2292 mmol) were dissolved in dry DCM (0.41 mL) at rt. DCC (0.15 mL, 0.1475 mmol) and 4-dimethylaminopyridine (3.00 mg, 0.0246 mmol) were successively added and the reaction mixture was stirred at rt overnight. DCM was removed under reduced pressure and the residue purified by reverse phase column chromatography (eluent: 20-70% MeOH in water (+0.1% formic acid in both)). Further purification by ion exchange SCX-II column chromatography, washing with MeOH before eluting with 2M NH₃ in MeOH and then purification by silica gel column chromatography (eluent: DCM/EtOAc—6/4) afforded propyl (S)-1-methyl-3-(3-((7-(5-methyl-1,2,4-oxadiazol-3-yl)isoquinolin-1-yl)amino)pyrrolidine-1-carbonyl)-1H-pyrazole-5-carboxylate (2.81 mg, 4%, 0.0046 mmol) as a colorless solid. HPLC/MS m/z: 490.22, [M+H]⁺, Rt (T): 1.15 min. ¹H NMR (600 MHz, Chloroform-d) δ 8.53-8.46 (m, 1H), 8.26 (dt, J=8.5, 1.8 Hz, 1H), 8.05 (dd, J=35.9, 5.8 Hz, 1H), 7.79 (dd, J=8.5, 6.0 Hz, 1H), 7.09-6.98 (m, 2H), 5.50 (s, 1H), 4.97-4.86 (m, 1H), 4.34-4.20 (m, 3H), 4.20-4.15 (m, 3H), 3.97-3.89 (m, 1H), 3.86-3.79 (m, 1H), 3.76-3.59 (m, 1H), 2.73-2.70 (m, 3H), 2.54-2.43 (m, 1H), 2.28-2.14 (m, 1H), 1.84-1.75 (m, 2H), 1.03-0.95 (m, 3H). (Note: Two rotameric forms present).

### Example 246: (S)-(5-(tert-butyl)-4-methylthiazol-2-yl)(3-((7-(5-methyl-1,2,4-oxadiazol-3-yl)isoquinolin-1-yl)amino)pyrrolidin-1-yl)methanone

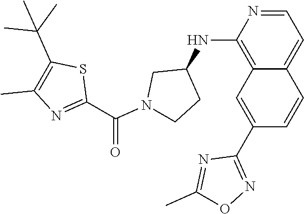 [SMILES: Cc1nc(-c2ccc3ccnc(N[C@H]4CCN(C(=O)c5nc(C)c(C(C)(C)C)s5)C4)c3c2)no1]

Example 246.1: To a solution of copper(I) bromide (365.20 mg, 2.5458 mmol) and isoamyl nitrite (0.25 mL, 1.8669 mmol) in acetonitrile (7.1 mL) was added 5-tert-butyl-4-methyl-thiazol-2-amine (289.00 mg, 1.6972 mmol) portion wise. The reaction mixture was stirred for 1 h at rt. Purification by silica gel column chromatography (eluent: 1-10% diethyl ether in cyclohexane) to afford 2-bromo-5-tert-butyl-4-methyl-thiazole (134 mg, 34%, 0.572 mmol).

Example 246.2: To a solution of 2-bromo-5-tert-butyl-4-methyl-thiazole (59.00 mg, 0.2520 mmol) in dry THF (1.94 mL) containing molecular sieves at −78° C. nBuLi (0.20 mL, 0.2772 mmol) was added and the mixture was stirred at −78° C. during 15 min. Dry ice (6 pieces) was added and the reaction stirred for 5 min at −78 C followed at rt for 1 h. EtOAc (6 mL) was added then washed with water (2×5 mL). The aqueous solution was acidified to pH=4 (HCl 1 M) then evaporated under vacuum. Crude was washed with a solution of ACN/H₂O 50/50 with some drops of HCl (1 M) pH=1 to afford 5-tert-butyl-4-methyl-thiazole-2-carboxylic acid (36 mg, 72%, 0.181 mmol). HPLC/MS m/z: 200.08, [M+H]⁺, Rt (P): 1.22 min.

Example 246.3: To a stirring solution containing 5-tert-butyl-4-methyl-thiazole-2-carboxylic acid (36.00 mg, 0.1807 mmol), 7-(5-methyl-1,2,4-oxadiazol-3-yl)-N-[(3S)-pyrrolidin-3-yl]isoquinolin-1-amine (58.69 mg, 0.1987 mmol) and HATU (128.45 mg, 0.3378 mmol) in DMF (0.90 mL) was added DIPEA (0.10 mL, 0.5962 mmol). The resulting solution was stirred at rt overnight. Purified by reverse phase column chromatography (eluent: 20-80% MeOH in water (+0.1% formic acid in both)) to afford (S)-(5-(tert-butyl)-4-methylthiazol-2-yl)(3-((7-(5-methyl-1,2,4-oxadiazol-3-yl)isoquinolin-1-yl)amino)pyrrolidin-1-yl)methanone (34 mg, 39%, 0.0713 mmol) as a yellow solid. HPLC/MS m/z: 477.21, [M+H]⁺, Rt (T): 2.88 min. 1 H NMR (600 MHz, DMSO-d₆) δ 9.03 (d, J=15.8 Hz, 1H), 8.18 (d, J=8.5 Hz, 1H), 7.99 (t, J=6.5 Hz, 1H), 7.88 (dd, J=8.7, 3.2 Hz, 1H), 7.05 (d, J=4.8 Hz, 1H), 4.83 (sext, J=5.9 Hz, 0.5H), 4.72 (sext, J=5.6 Hz, 0.5H), 4.38 (dd, J=12.3, 6.4 Hz, 0.5H), 4.27 (dt, J=11.9, 7.3 Hz, 0.5H), 4.13 (ddt, J=18.8, 13.4, 5.5 Hz, 1H), 3.93 (dd, J=12.8, 6.5 Hz, 0.5H), 3.77 (dt, J=12.3, 7.2 Hz, 0.5H), 3.71 (dd, J=12.8, 4.4 Hz, 0.5H), 3.64 (ddd, J=13.0, 7.9, 6.1 Hz, 0.5H), 2.70 (d, J=1.8 Hz, 3H), 2.50 (s, 1.5H), 2.46 (s, 1.5H), 2.33 (dq, J=13.6, 6.8 Hz, 0.5H), 2.24 (dq, J=13.5, 6.8 Hz, 1H), 2.11 (dq, J=12.9, 6.5 Hz, 0.5H), 1.40 (s, 4.5H), 1.39 (s, 5H), 1.30-1.20 (m, 0.5H). (Note: Two rotameric forms present).

The following examples were prepared analogously:

### Example 247: (S)-(5-cyclopropyl-1-methyl-1 H-pyrazol-3-yl)(3-((7-(5-methyl-1,2,4-oxadiazol-3-yl)isoquinolin-1-yl)amino)pyrrolidin-1-yl)methanone

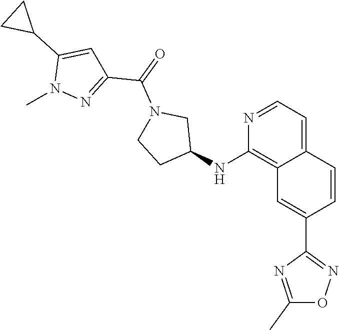 [SMILES: Cc1nc(-c2ccc3ccnc(N[C@H]4CCN(C(=O)c5cc(C6CC6)n(C)n5)C4)c3c2)no1]

24 mg (80%), colorless solid. HPLC/MS m/z: 444.21 [M+H]⁺, Rt (U): 2.20 min. ¹H NMR (600 MHz, DMSO-d₆) δ 9.06-8.98 (m, 1H), 8.16 (dd, J=8.5, 1.5 Hz, 1H), 8.00 (dd, J=13.3, 5.7 Hz, 1H), 7.91-7.81 (m, 2H), 7.05-6.99 (m, 1H), 6.26 (d, J=7.1 Hz, 1H), 4.83-4.66 (m, 1H), 4.29-4.08 (m, 1H), 4.00-3.93 (m, 0.5H), 3.92-3.84 (m, 4H), 3.79-3.72 (m, 0.5H), 3.66-3.54 (m, 1H), 2.75-2.68 (m, 3H), 2.34-2.01 (m, 2H), 1.97-1.82 (m, 1H), 1.03-0.87 (m, 2H), 0.72-0.61 (m, 2H). (Note: Two rotameric forms present).

### Example 248: (S)-(3-((7-(5-methyl-1,2,4-oxadiazol-3-yl)isoquinolin-1-yl)amino)pyrrolidin-1-yl)(1-methyl-5-phenyl-1 H-pyrazol-3-yl)methanone

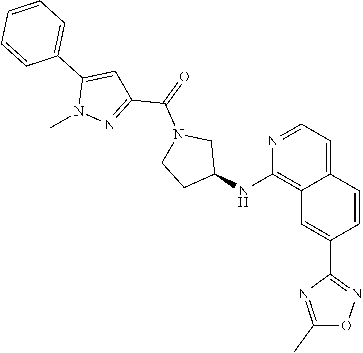 [SMILES: Cc1nc(-c2ccc3ccnc(N[C@H]4CCN(C(=O)c5cc(-c6ccccc6)n(C)n5)C4)c3c2)no1]

22.6 mg (70%), colorless solid. HPLC/MS m/z: 480.22, [M+H]⁺, Rt (U): 2.48 min. ¹H NMR (600 MHz, DMSO-d₆) δ 9.07-9.00 (m, 1H), 8.21-8.14 (m, 1H), 8.06-7.98 (m, 1H), 7.91-7.84 (m, 2H), 7.62-7.43 (m, 6H), 7.06-7.00 (m, 1H), 6.82-6.76 (m, 1H), 4.87-4.71 (m, 1H), 4.34 (dd, J=11.7, 6.5 Hz, 0.5H), 4.20 (dt, J=11.5, 7.1 Hz, 0.5H), 4.05 (ddd, J=11.5, 7.5, 6.0 Hz, 0.5H), 3.99-3.92 (m, 2.5H), 3.89 (s, 1.5H), 3.81 (ddd, J=12.1, 7.8, 6.1 Hz, 0.5H), 3.72-3.60 (m, 1H), 2.75-2.68 (m, 3H), 2.38-2.10 (m, 2H). (Note: Two rotameric forms present).

### Example 249: (S)-(5-isopropyl-1-methyl-1 H-pyrazol-3-yl)(3-((7-(5-methyl-1,2,4-oxadiazol-3-yl)isoquinolin-1-yl)amino)pyrrolidin-1-yl)methanone

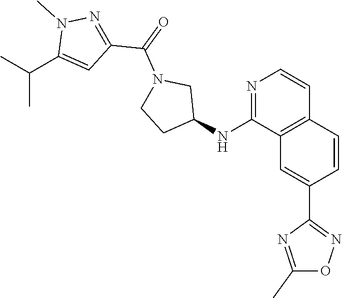 [SMILES: Cc1nc(-c2ccc3ccnc(N[C@H]4CCN(C(=O)c5cc(C(C)C)n(C)n5)C4)c3c2)no1]

Example 249.1: 5-Methyl-3-(2-oxidoisoquinolin-2-ium-7-yl)-1,2,4-oxadiazole [Example 96](750.00 mg, 3.3008 mmol) was dissolved/suspended in DCM (11.00 mL) and (S)-(−)-1-Boc-3-aminopyrrolidine (0.72 mL, 4.126 mmol) was added, quickly followed by DIPEA (1.72 mL, 9.9023 mmol). To this solution was added PyBroP (1.72 g, 3.6969 mmol) and the mixture was stirred for 20 h. The solvent was removed. Purification by silica gel column chromatography (eluent: 15-50% EtOAc in cyclohexane) afforded tert-butyl (3S)-3-[[7-(5-methyl-1,2,4-oxadiazol-3-yl)-1-isoquinolyl]amino]pyrrolidine-1-carboxylate (947 mg, 73%, 2.3947 mmol) as a yellow powder. HPLC/MS m/z: 396.2, [M+H]⁺, Rt (P): 1.31 min.

Example 249.2: tert-Butyl (3S)-3-[[7-(5-methyl-1,2,4-oxadiazol-3-yl)-1-isoquinolyl]amino]pyrrolidine-1-carboxylate (947.00 mg, 2.3947 mmol) was dissolved in dry DCM (12.0 mL). TFA (1.83 mL, 23.947 mmol) was added, and the reaction mixture was stirred at rt for 4 h. The solvent was removed in vacuo and the residue purified by ion exchange SCX-II column chromatography, washing with MeOH before eluting with 2M NH₃ in MeOH. Purified by reverse phase column chromatography (eluent: 0-50% MeOH/H₂O+0.1% formic acid) followed by purification by ion exchange SCX-II column chromatography, washing with MeOH before eluting with 2M NH₃ in MeOH afforded 7-(5-methyl-1,2,4-oxadiazol-3-yl)-N-[(3S)-pyrrolidin-3-yl]isoquinolin-1-amine (410 mg, 58%, 1.3882 mmol) as a colorless powder. HPLC/MS m/z: 296.2, [M+H]⁺, Rt (T): 0.67 min.

Example 249.3: To 7-(5-methyl-1,2,4-oxadiazol-3-yl)-N-[(3S)-pyrrolidin-3-yl]isoquinolin-1-amine (15.00 mg, 0.0508 mmol) and 1-methyl-5-(propan-2-yl)-1 H-pyrazole-3-carboxylic acid (12.81 mg, 0.0762 mmol) in anhydrous DMF (0.51 mL) were added 1-propanephosphonic anhydride (0.12 mL, 0.1016 mmol) and triethyl-amine (0.01 mL, 0.1016 mmol). The vial was capped and stirred at rt overnight. Purified by reverse phase column chromatography (eluent: 10-80% MeOH in water (+0.1% formic acid modifier in both)) followed by ion-exchange column SCX-II, eluting with 2M NH₃ in MeOH, to afford (S)-(5-isopropyl-1-methyl-1 H-pyrazol-3-yl)(3-((7-(5-methyl-1,2,4-oxadiazol-3-yl)isoquinolin-1-yl)amino)pyrrolidin-1-yl)methanone (10 mg, 44%, 0.0224 mmol). HPLC/MS m/z: 446.2 [M+H]⁺, Rt (U): 2.29 min. ¹H NMR (600 MHz, DMSO-d₆) δ 9.03 (s, 0.5H), 9.01 (s, 0.5H), 8.16 (dt, J=8.5, 1.5 Hz, 1H), 8.02 (d, J=5.7 Hz, 0.5H), 7.99 (d, J=5.7 Hz, 0.5H), 7.90-7.82 (m, 2H), 7.04-7.00 (m, 1H), 6.46-6.42 (m, 1H), 4.80 (sext, J=6.3 Hz, 0.5H), 4.71 (sext, J=5.8 Hz, 0.5H), 4.28 (dd, J=11.8, 6.5 Hz, 0.5H), 4.15 (dt, J=11.6, 7.1 Hz, 0.5H), 4.02-3.95 (m, 0.5H), 3.89 (ddd, J=14.2, 8.0, 6.2 Hz, 1H), 3.83 (s, 1.5H), 3.79 (s, 1.5H), 3.78-3.55 (m, 1.5H), 3.08-2.98 (m, 1H), 2.71 (d, J=1.7 Hz, 3H), 2.33-2.05 (m, 2H), 1.24-1.17 (m, 6H). (Note: Rotameric forms present).

The following examples were prepared by an analogous procedure:

### Example 250: (S)-(1 H-imidazol-5-yl)(3-((7-(5-methyl-1,2,4-oxadiazol-3-yl)isoquinolin-1-yl)amino)pyrrolidin-1-yl)methanone

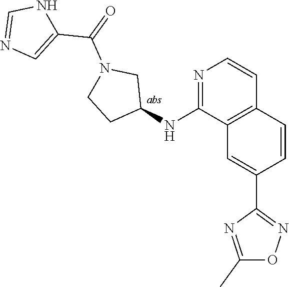 [SMILES: Cc1nc(-c2ccc3ccnc(N[C@H]4CCN(C(=O)c5cnc[nH]5)C4)c3c2)no1]

6 mg (35%), colorless powder. HPLC/MS m/z: 390.2, [M+H]⁺, Rt (T): 0.73 min. ¹H NMR (600 MHz, Methanol-d₄) δ 8.91 (d, J=8.8 Hz, 1H), 8.19 (dd, J=8.5, 2.2 Hz, 1H), 7.94 (dd, J=16.9, 5.9 Hz, 1H), 7.82-7.70 (m, 2H), 7.69-7.57 (m, 1H), 7.03-6.98 (m, 1H), 4.83-4.76 (m, 1H), 4.43-4.12 (m, 1H), 4.12-3.85 (m, 2H), 3.82-3.71 (m, 1H), 2.67 (s, 3H), 2.51-2.17 (m, 2H).

### Example 251: (5-Methylisoxazol-3-yl)-[(3S)-3-[[7-(5-methyl-1,2,4-oxadiazol-3-yl)-1-isoquinolyl]amino]pyrrolidin-1-yl]methanone

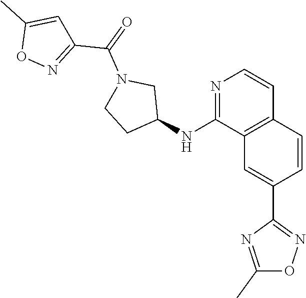 [SMILES: Cc1cc(C(=O)N2CC[C@H](Nc3nccc4ccc(-c5noc(C)n5)cc34)C2)no1]

7 mg (34%), colorless amorphous powder. HPLC/MS m/z: 405.17 [M+H]⁺, Rt (T): 1.03 min. ¹H NMR (600 MHz, DMSO-d₆) δ 9.03 (s, 1H), 9.00 (s, 1H), 8.17 (ddd, J=8.5, 3.2, 1.5 Hz, 2H), 8.01 (dd, J=19.2, 5.7 Hz, 2H), 7.92-7.82 (m, 4H), 7.03 (dd, J=8.6, 5.7 Hz, 2H), 6.52 (dd, J=9.3, 1.1 Hz, 2H), 4.83-4.70 (m, 2H), 4.12 (dd, J=11.5, 6.5 Hz, 1H), 4.00-3.91 (m, 2H), 3.88-3.74 (m, 3H), 3.73-3.60 (m, 2H), 2.71 (s, 6H), 2.47 (s, 3H), 2.45 (s, 3H), 2.36-2.27 (m, 2H), 2.24-2.13 (m, 2H). (Note: Two rotameric forms present).

### Example 252: (1-tert-Butylpyrazol-4-yl)-[(3S)-3-[[7-(5-methyl-1,2,4-oxadiazol-3-yl)-1-isoquinolyl]amino]pyrrolidin-1-yl]methanone

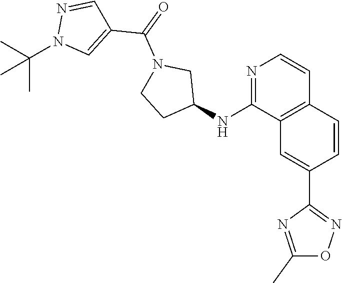 [SMILES: Cc1nc(-c2ccc3ccnc(N[C@H]4CCN(C(=O)c5cnn(C(C)(C)C)c5)C4)c3c2)no1]

15 mg (50%), colorless, amorphous powder. HPLC/MS m/z: 446.23 [M+H]⁺, Rt (T): 1.06 min. ¹H NMR (600 MHz, DMSO-d₆) δ 9.01 (d, J=15.5 Hz, 2H), 8.20-8.10 (m, 4H), 8.00 (dd, J=16.7, 5.7 Hz, 2H), 7.92-7.74 (m, 6H), 7.02 (dd, J=8.0, 5.6 Hz, 2H), 4.81 (q, J=5.9 Hz, 1H), 4.71 (q, J=5.8 Hz, 1H), 4.15 (dd, J=10.5, 6.6 Hz, 1H), 3.98-3.86 (m, 2H), 3.86-3.80 (m, 1H), 3.73 (ddd, J=26.1, 12.0, 6.1 Hz, 2H), 3.63 (dd, J=12.3, 5.0 Hz, 1H), 3.57 (dt, J=12.5, 7.1 Hz, 1H), 2.70 (s, 6H), 2.33 (dt, J=13.4, 6.5 Hz, 1H), 2.23 (dt, J=12.9, 6.5 Hz, 2H), 2.13 (dq, J=13.1, 6.8 Hz, 1H), 1.54 (s, 9H), 1.52 (s, 9H). (Note: Two rotameric forms present).

### Example 253: (S)-(5-isopropyl-1 H-pyrazol-3-yl)(3-((7-(5-methyl-1,2,4-oxadiazol-3-yl)isoquinolin-1-yl)amino)pyrrolidin-1-yl)methanone

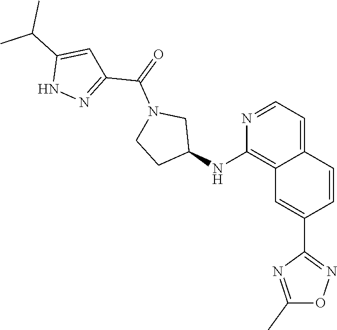 [SMILES: Cc1nc(-c2ccc3ccnc(N[C@H]4CCN(C(=O)c5cc(C(C)C)[nH]n5)C4)c3c2)no1]

9 mg (28%), colorless powder. HPLC/MS m/z: 432.2, [M+H]⁺, Rt (U): 2.19 min. ¹H NMR (600 MHz, DMSO-d₆) δ 12.92 (s, 0.5H), 12.87 (s, 0.5H), 9.03 (s, 0.5H), 9.01 (s, 0.5H), 8.20-8.13 (m, 1H), 8.01 (d, J=5.7 Hz, 0.5H), 7.99 (d, J=5.7 Hz, 0.5H), 7.89-7.82 (m, 2H), 7.04-6.99 (m, 1H), 6.41-6.37 (m, 1H), 4.76-4.69 (m, 1H), 4.30 (m, 0.5H), 4.16-4.08 (m, 0.5H), 4.04-3.96 (m, 0.5H), 3.96-3.87 (m, 1H), 3.81-3.73 (m, 0.5H), 3.69-3.55 (m, 1H), 2.99-2.92 (m, 1H), 2.70 (s, 1.5H), 2.70 (s, 1.5H), 2.28-2.08 (m, 2H), 1.25-1.18 (m, 6H). (Note: Rotameric forms present).

### Example 254: (S)-(5-(tert-butyl)-1 H-pyrazol-3-yl)(3-((7-(5-methyl-1,2,4-oxadiazol-3-yl)isoquinolin-1-yl)amino)pyrrolidin-1-yl)methanone

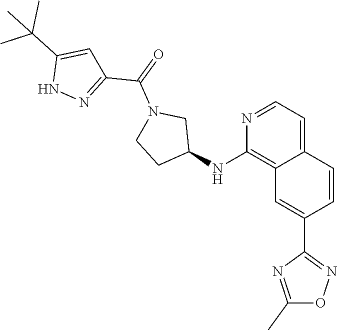 [SMILES: Cc1nc(-c2ccc3ccnc(N[C@H]4CCN(C(=O)c5cc(C(C)(C)C)[nH]n5)C4)c3c2)no1]

10 mg (30%), colorless powder. HPLC/MS m/z: 446.2, [M+H]⁺, Rt (T): 2.33 min. ¹H NMR (600 MHz, DMSO-d₆) δ 12.94 (s, 0.5H), 12.89 (s, 0.5H), 9.03 (s, 0.5H), 9.01 (s, 0.5H), 8.18-8.14 (m, 1H), 8.02 (d, J=5.7 Hz, 0.5H), 7.99 (d, J=5.7 Hz, 0.5H), 7.90-7.80 (m, 2H), 7.04-6.99 (m, 1H), 6.41-6.36 (m, 1H), 4.78-4.68 (m, 1H), 4.35-4.28 (m, 0.5H), 4.19-4.09 (m, 0.5H), 4.04-3.88 (m, 1.5H), 3.81-3.74 (m, 0.5H), 3.70-3.56 (m, 1H), 2.71-2.70 (m, 3H), 2.27-2.08 (m, 2H), 1.28 (s, 4.5H), 1.26 (s, 4.5H). (Note: Rotameric forms present).

### Example 255: Propyl 4-methyl-2-((2S,3R)-2-methyl-3-((7-(5-methyl-1,2,4-oxadiazol-3-yl)isoquinolin-1-yl)amino)pyrrolidine-1-carbonyl)thiazole-5-carboxylate

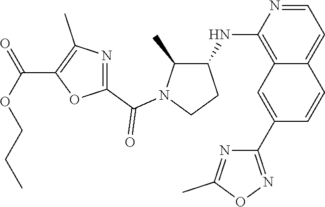 [SMILES: CCCOC(=O)c1oc(C(=O)N2CC[C@@H](Nc3nccc4ccc(-c5noc(C)n5)cc34)[C@@H]2C)nc1C]

tert-Butyl (2R,3S)-3-amino-2-methylpyrrolidine-1-carboxylate was used to prepare propyl 4-methyl-2-((2S,3R)-2-methyl-3-((7-(5-methyl-1,2,4-oxadiazol-3-yl)isoquinolin-1-yl)amino)pyrrolidine-1-carbonyl)thiazole-5-carboxylate (11 mg, 20%, 0.0211 mmol). HPLC/MS m/z: 521.20, [M+H]⁺, Rt (Q): 2.86 min. ¹H NMR (600 MHz, Chloroform-d) δ 8.58 (d, J=48.9 Hz, 1H), 8.21 (dd, J=8.5, 1.5 Hz, 1H), 8.08 (dd, J=5.8, 2.3 Hz, 1H), 7.75 (dd, J=8.5, 1.3 Hz, 1H), 7.00-6.97 (m, 1H), 5.95 (d, J=5.8 Hz, 0.5H), 5.90 (d, J=6.1 Hz, 0.5H), 5.29 (q, J=6.4 Hz, 0.5H), 4.73 (t, J=5.8 Hz, 0.5H), 4.62-4.55 (m, 1H), 4.43 (ddd, J=12.2, 8.8, 3.1 Hz, 0.5H), 4.33 (ddd, J=12.7, 9.8, 7.3 Hz, 0.5H), 4.26-4.20 (m, 2H), 3.99 (ddd, J=13.4, 10.1, 7.9 Hz, 0.5H), 3.89 (ddd, J=12.9, 9.6, 2.5 Hz, 0.5H), 2.70 (s, 3H), 2.60 (d, J=4.0 Hz, 3H), 2.58-2.45 (m, 0.5H), 2.32-2.17 (m, 0.5H), 1.74 (ddd, J=14.0, 6.9, 3.1 Hz, 2H), 1.48 (dd, J=15.2, 6.5 Hz, 3H), 0.99 (td, J=7.4, 1.2 Hz, 3H). (Note: 1×NH not observed; two rotameric forms present).

### Example 256: tert-Butyl (3R,4R)-3-amino-4-fluoropyrrolidine-1-carboxylate was used to prepare propyl 2-((3R,4R)-3-fluoro-4-((7-(5-methyl-1,2,4-oxadiazol-3-yl)isoquinolin-1-yl)amino)pyrrolidine-1-carbonyl)-4-methylthiazole-5-carboxylate

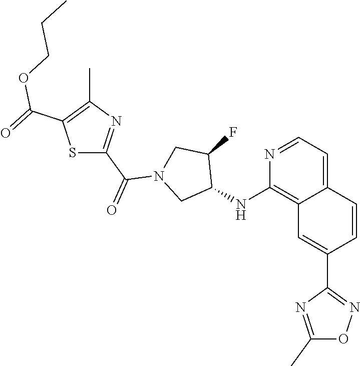 [SMILES: CCCOC(=O)c1sc(C(=O)N2C[C@@H](F)[C@H](Nc3nccc4ccc(-c5noc(C)n5)cc34)C2)nc1C]

14 mg (25%), yellow solid. HPLC/MS m/z: 525.17, [M+H]⁺, Rt (Q): 3.15 min. ¹H NMR (600 MHz, Chloroform-d) δ 8.69 (s, 0.5H), 8.64 (d, J=1.5 Hz, 0.5H), 8.24 (dd, J=8.5, 1.5 Hz, 1H), 8.13 (dd, J=5.8, 1.2 Hz, 1H), 7.78 (d, J=8.5 Hz, 1H), 7.06 (d, J=5.8 Hz, 1H), 6.04-5.94 (m, 1H), 5.47 (dddd, J=76.1, 50.1, 3.6, 1.6 Hz, 1H), 5.12 (dt, J=10.4, 5.6 Hz, 0.5H), 4.99-4.91 (m, 0.5H), 4.76 (d, J=13.5 Hz, 0.5H), 4.64 (dd, J=25.2, 14.9 Hz, 0.5H), 4.55 (dd, J=13.5, 5.6 Hz, 0.5H), 4.43 (ddd, J=40.2, 14.9, 3.5 Hz, 0.5H), 4.27-4.21 (m, 3H), 4.18-3.99 (m, 1H), 2.73 (s, 1.5H), 2.67 (s, 1.5H), 2.59 (s, 1.5H), 2.58 (s, 1.5H), 1.79-1.69 (m, 2H), 0.99 (dt, J=8.6, 7.4 Hz, 3H). (Note: Two rotameric forms present).

### Example 257: Propyl 2-methyl-4-(3-{[7-(5-methyl-1,2,4-oxadiazol-3-yl)isoquinolin-1-yl]amino}propanamido)-1 H-imidazole-1-carboxylate

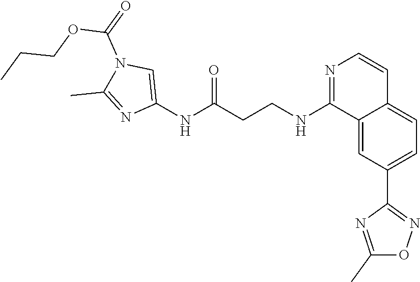 [SMILES: CCCOC(=O)n1cc(NC(=O)CCNc2nccc3ccc(-c4noc(C)n4)cc23)nc1C]

Example 257.1: A solution of benzyl N-(3-chloro-3-oxopropyl)carbamate (990.0 mg, 4.096 mmol) and 2-methyl-1 H-imidazol-4-amine (950.0 mg, 9.782 mmol) in THE (13.0 mL) was stirred for 2 h at rt. The resulting mixture was concentrated under vacuum. The residue was purified by silica gel column chromatography (eluent: petroleum ether/ethyl acetate—1:1) to afford benzyl N-[2-[(2-methyl-1 H-imidazol-4-yl)carbamoyl]ethyl]carbamate (962 mg, 78%) as a colorless solid. HPLC/MS m/z: 303.1 [M+H]⁺, Rt (A): 0.54 min.

Example 257.2: To a solution of benzyl N-[2-[(2-methyl-1 H-imidazol-4-yl)carbamoyl], ethyl], carbamate (866.0 mg, 2.864 mmol) in MeOH (8.00 mL) was added Pd/C (300 mg, 0.282 mmol) under nitrogen atmosphere, and the mixture was hydrogenated at rt for 2 h under hydrogen atmosphere. The reaction mixture was filtered through a celite pad and concentrated under reduced pressure to afford 3-amino-N-(2-methyl-1 H-imidazol-4-yl)propanamide (452 mg, 94%) as a yellow solid. HPLC/MS m/z: 169.25 [M+H]⁺, Rt (A): 0.1 min.

Example 257.3: To a stirred solution of 3-amino-N-(2-methyl-1 H-imidazol-4-yl)propenamide (0.95 g, 5.648 mmol) and 7-(5-methyl-1,2,4-oxadiazol-3-yl)isoquinolin-2-ium-2-olate (1.43 g, 6.293 mmol) in DMF (5.0 mL) were added diethylamine (2.15 g, 16.675 mmol) and PyBrOP (3.42 g, 7.336 mmol) at rt, and the resulting mixture was stirred for 2 h at rt. The mixture was concentrated under vacuum, and the residue was purified by Prep-TLC (eluent: petroleum ether/ethyl acetate—1:1) to afford 3-[[7-(5-methyl-1,2,4-oxadiazol-3-yl)isoquinolin-1-yl]amino]-N-(2-methyl-1 H-imidazol-4-yl)propanamide (757 mg, 36%) as a yellow solid. HPLC/MS m/z: 378.10 [M+H]⁺, Rt (A): 0.47 min.

Example 257.4: To a stirred solution of 3-[[7-(5-methyl-1,2,4-oxadiazol-3-yl)isoquinolin-1-yl]amino]-N-(2-methyl-1H-imidazol-4-yl)propanamide (90.0 mg, 0.238 mmol) in dichloromethane (2.0 mL) were added DMAP (9.5 mg, 0.078 mmol), triethylamine (114 mg, 1.127 mmol) and carbonochloridic acid, propyl ester (38.0 mg, 0.310 mmol) and the resulting mixture was stirred at rt for 1 h. The resulting mixture was concentrated under vacuum, and the residue was purified by Prep-HPLC to give propyl 2-methyl-4-(3-{[7-(5-methyl-1,2,4-oxadiazol-3-yl)isoquinolin-1-yl]amino}propanamido)-1 H-imidazole-1-carboxylate (13.8 mg, 13%) as a colorless solid. HPLC/MS m/z: 464.25 [M+H]⁺, Rt (B): 1.39 min; ¹H NMR (300 MHz, DMSO-d₆) δ 10.56 (s, 1H), 8.89 (s, 1H), 8.20-8.10 (m, 1H), 7.98 (t, J=5.8 Hz, 2H), 7.85 (d, J=8.5 Hz, 1H), 7.50 (s, 1H), 6.97 (d, J=5.7 Hz, 1H), 4.29 (t, J=6.5 Hz, 2H), 3.76 (q, J=6.6 Hz, 2H), 2.79-2.71 (m, 2H), 2.71 (s, 3H), 2.49 (s, 3H), 1.87-1.66 (m, 2H), 0.97 (t, J=7.4 Hz, 3H).

The following examples were prepared analogously:

### Example 258: Propan-2-yl 2-methyl-4-(3-{[7-(5-methyl-1,2,4-oxadiazol-3-yl)isoquinolin-1-yl]amino}propanamido)-1 H-imidazole-1-carboxylate

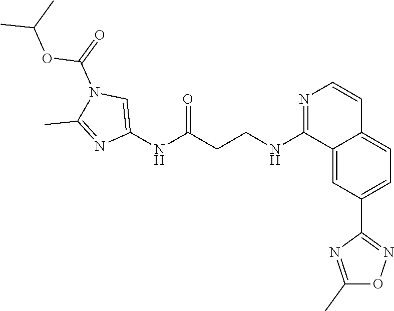 [SMILES: Cc1nc(-c2ccc3ccnc(NCCC(=O)Nc4cn(C(=O)OC(C)C)c(C)n4)c3c2)no1]

18.5 mg (8%) colorless solid. HPLC/MS m/z: 464.10 [M+H]⁺, Rt (C): 0.72 min; ¹H NMR (400 MHz, DMSO-d₆): δ 10.54 (s, 1H), 8.88 (s, 1H), 8.18-8.11 (m, 1H), 7.98 (s, 1H), 7.96 (d, J=5.2 Hz, 1H), 7.85 (d, J=8.5 Hz, 1H), 7.47 (s, 1H), 6.96 (d, J=5.8 Hz, 1H), 5.13-5.01 (m, 1H), 3.80-3.71 (m, 2H), 2.73 (t, J=7.0 Hz, 2H), 2.70 (s, 3H), 2.48 (s, 3H), 1.35 (d, J=6.2 Hz, 6H).

### Example 259: Ethyl 2-methyl-4-(3-{[7-(5-methyl-1,2,4-oxadiazol-3-yl)isoquinolin-1-yl]amino}propanamido)-1 H-imidazole-1-carboxylate

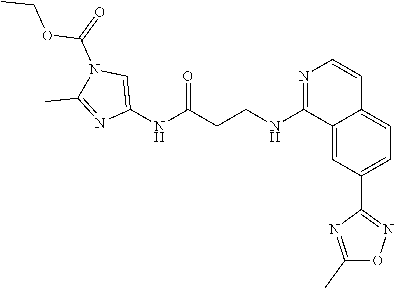 [SMILES: CCOC(=O)n1cc(NC(=O)CCNc2nccc3ccc(-c4noc(C)n4)cc23)nc1C]

21.4 mg (9%) colorless solid. HPLC/MS m/z: 450.10 [M+H]⁺, Rt (D): 1.33 min; ¹H NMR (400 MHz, DMSO-d₆): δ 10.55 (s, 1H), 8.89 (s, 1H), 8.19-8.12 (m, 1H), 7.97 (d, J=5.8 Hz, 2H), 7.85 (d, J=8.5 Hz, 1H), 7.48 (s, 1H), 6.97 (d, J=5.8 Hz, 1H), 4.37 (q, J=7.1 Hz, 2H), 3.80-3.71 (m, 2H), 2.73 (t, J=7.0 Hz, 2H), 2.70 (s, 3H), 2.48 (s, 3H), 1.34 (t, J=7.1 Hz, 3H).

### Example 260: Propyl 4-(3-{[7-(5-methyl-1,2,4-oxadiazol-3-yl)isoquinolin-1-yl]amino}propanamido)-1 H-imidazole-1-carboxylate

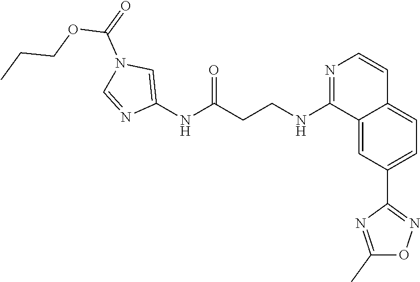 [SMILES: CCCOC(=O)n1cnc(NC(=O)CCNc2nccc3ccc(-c4noc(C)n4)cc23)c1]

37.7 mg (9%) colorless solid. HPLC/MS m/z: 450.15 [M+H]⁺, Rt (C): 0.82 min; ¹H NMR (300 MHz, DMSO-d₆): δ 10.69 (s, 1H), 8.87 (s, 1H), 8.16-8.09 (m, 2H), 7.96 (d, J=5.7 Hz, 2H), 7.83 (d, J=8.5 Hz, 1H), 7.54 (d, J=1.6 Hz, 1H), 6.95 (d, J=5.8 Hz, 1H), 4.30 (t, J=6.6 Hz, 2H), 3.74 (t, J=6.1 Hz, 2H), 2.74 (t, J=6.9 Hz, 2H), 2.68 (s, 3H), 1.72 (sext, J=7.1 Hz, 2H), 0.94 (t, J=7.4 Hz, 3H).

### Example 261: Ethyl 2-methyl-4-(3-{[7-(1-methyl-1 H-pyrazol-4-yl)isoquinolin-1-yl]amino}propanamido)-1 H-imidazole-1-carboxylate

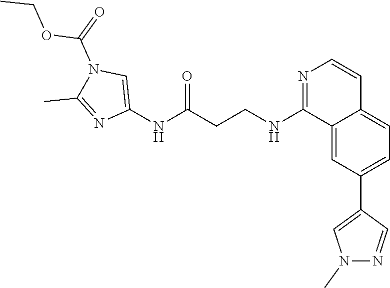 [SMILES: CCOC(=O)n1cc(NC(=O)CCNc2nccc3ccc(-c4cnn(C)c4)cc23)nc1C]

1.6 mg (5%) off-white solid. HPLC/MS m/z: 448.05 [M+H]⁺, Rt (E): 0.55 min; ¹H NMR (400 MHz, DMSO-d₆): δ 10.57 (s, 1H), 8.38 (s, 1H), 8.21 (s, 1H), 8.00 (s, 1H), 7.86-7.78 (m, 2H), 7.70-7.64 (m, 1H), 7.52-7.44 (m, 2H), 6.86 (d, J=5.8 Hz, 1H), 4.37 (q, J=7.1 Hz, 2H), 3.90 (s, 3H), 3.81-3.74 (m, 2H), 2.73 (t, J=7.0 Hz, 2H), 2.48 (s, 3H), 1.34 (t, J=7.1 Hz, 3H).

### Example 262: Propyl 2-methyl-4-(3-{[7-(1-methyl-1 H-pyrazol-4-yl)isoquinolin-1-yl]amino}propanamido)-1 H-imidazole-1-carboxylate

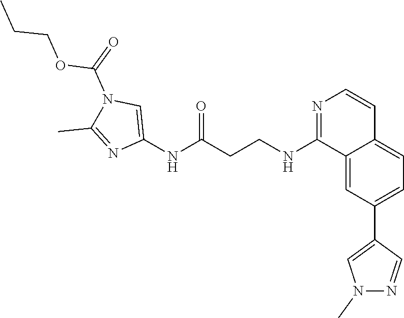 [SMILES: CCCOC(=O)n1cc(NC(=O)CCNc2nccc3ccc(-c4cnn(C)c4)cc23)nc1C]

1.1 mg (3%) off-white solid. HPLC/MS m/z: 462.15 [M+H]⁺, Rt (E): 0.59 min; ¹H NMR (400 MHz, DMSO-d₆): δ 10.58 (s, 1H), 8.39 (s, 1H), 8.21 (s, 1H), 8.00 (s, 1H), 7.87-7.79 (m, 2H), 7.67 (d, J=8.5 Hz, 1H), 7.50 (s, 2H), 6.86 (d, J=5.8 Hz, 1H), 4.29 (t, J=6.5 Hz, 2H), 3.89 (s, 3H), 3.81-3.73 (m, 2H), 2.73 (t, J=6.9 Hz, 2H), 2.48 (s, 3H), 1.80-1.67 (m, 2H), 0.96 (t, J=7.4 Hz, 3H).

### Example 263: Propyl 2-methyl-4-(3-{[3-(5-methyl-1,2,4-oxadiazol-3-yl)phenyl]formamidolpropanamido)-1 H-imidazole-1-carboxylate

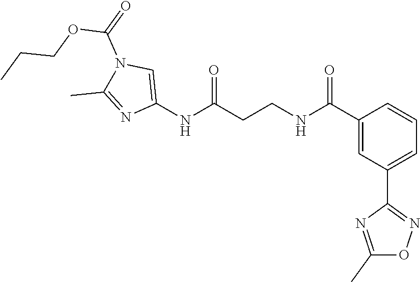 [SMILES: CCCOC(=O)n1cc(NC(=O)CCNC(=O)c2cccc(-c3noc(C)n3)c2)nc1C]

Example 263.1: 3-(5-Methyl-1,2,4-oxadiazol-3-yl)benzoic acid (147.0 mg, 0.720 mmol) and methyl 3-aminopropanoate dihydrochloride (126.7 mg, 0.720 mmol) were dissolved in DMF (2.3 mL). HATU (260.1 mg, 0.684 mmol) and N-Ethyldiiso-propylamine (489,7 μl, 2.880 mmol) were added and the clear yellow solution was stirred for 1 h at rt. The reaction was concentrated in vacuo, and the residue was purified by RP-flash chromatography. The fractions were combined, diluted with saturated aqueous NaHCO₃-solution and extracted with dichloromethane. The combined organic layers were dried over sodium sulfate, filtered, and concentrated in vacuo to give 206 mg (99%) of methyl 3-{[3-(5-methyl-1,2,4-oxadiazol-3-yl)phenyl]formamido}propanoate as a colorless solid. HPLC/MS m/z: 289.9 [M+H]⁺, Rt (F): 0.83 min.

Example 263.2: Methyl 3-{[3-(5-methyl-1,2,4-oxadiazol-3-yl)phenyl]formamido}-propanoate (413.0 mg, 1.428 mmol) was dissolved in THF (18.0 mL) and water (9.0 mL). While stirring lithium hydroxide (85.5 mg, 3.569 mmol) was added and the reaction mixture was stirred at rt overnight. The reaction mixture was diluted with water, acidified to pH 3-4 with 0.1 N HCl solution, and extracted with ethyl acetate. The combined organic layers were washed with brine, dried over sodium sulfate, filtered, and concentrated in vacuo. The residue was purified by RP-flash chromatography to afford 390 mg (99%) of 3-{[3-(5-methyl-1,2,4-oxadiazol-3-yl)phenyl]formamido}propanoic acid as a colorless solid. HPLC/MS m/z: 275.9 [M+H]⁺, Rt (F): 0.77 min.

Example 263.3: To a stirred solution of 3-{[3-(5-methyl-1,2,4-oxadiazol-3-yl)phenyl]-formamido}-propanoic acid (249.0 mg, 0.904 mmol) in dichloromethane (4.0 mL) was added thionylchloride (161.5 mg, 1.357 mmol) at rt, and the resulting mixture was stirred for 1 h at rt. The reaction mixture was evaporated to dryness to afford 234 mg (88%) 3-[[3-(5-methyl-1,2,4-oxadiazol-3-yl)phenyl]formamido]propanoyl chloride as a colorless solid, which was used in the next step without further purification.

Example 263.4: To a stirred solution of 3-[[3-(5-methyl-1,2,4-oxadiazol-3-yl)phenyl]-formamido]-propanoyl chloride (90.0 mg, 0.306 mmol) and 2-methyl-1 H-imidazol-4-amine (95.0 mg, 0.977 mmol) in THF (2.0 mL) was added triethylamine (90.0 mg, 0.889 mmol) at rt, and the resulting mixture was stirred for 2 h at rt. The reaction mixture was concentrated under vacuum, and the residue was purified by silica gel column chromatography (eluent: dichloromethane/methanol—9:1) to afford 80 mg (74%) of 3-[[3-(5-methyl-1,2,4-oxadiazol-3-yl)phenyl]formamido]-N-(2-methyl-1 H-imidazol-4-yl)propanamide as a black solid. HPLC/MS m/z: 355.05 [M+H]⁺, Rt (A): 0.43 min.

Example 263.5: To a stirred solution of 3-[[3-(5-methyl-1,2,4-oxadiazol-3-yl)phenyl]formamido]-N-(2-methyl-1 H-imidazol-4-yl)propanamide (50.0 mg, 0.141 mmol) and carbonochloridic acid, propyl ester (19.0 mg, 0.155 mmol) in dichloromethane (2.0 mL) were added triethylamine (38 mg, 0.375 mmol) and DMAP (2 mg, 0.016 mmol) at rt, and the resulting mixture was stirred for 2 h at rt. The reaction mixture was concentrated under vacuum, and the crude product was purified by Prep-HPLC to afford 8.7 mg (14%) of propyl 2-methyl-4-(3-{[3-(5-methyl-1,2,4-oxadiazol-3-yl)phenyl]formamido}propanamido)-1 H-imidazole-1-carboxylate as a colorless solid. HPLC/MS m/z: 441.25 [M+H]⁺, Rt (G): 1.36 min. ¹H NMR (300 MHz, DMSO-d₆): δ 10.56 (s, 1H), 8.85-8.77 (m, 1H), 8.47 (t, J=1.7 Hz, 1H), 8.13 (d, J=7.7 Hz, 1H), 8.04 (d, J=7.9 Hz, 1H), 7.66 (t, J=7.8 Hz, 1H), 7.49 (s, 1H), 4.29 (t, J=6.5 Hz, 2H), 3.61-3.48 (m, 2H), 2.69 (s, 3H), 2.63 (t, J=7.0 Hz, 2H), 2.54-2.52 (s, 3H), 1.83-1.65 (m, 2H), 0.97 (t, J=7.4 Hz, 3H).

The following examples were prepared analogously:

### Example 264: Propan-2-yl 2-methyl-4-(3-{[3-(5-methyl-1,2,4-oxadiazol-3-yl)phenyl]formamidolpropan-amido)-1 H-imidazole-1-carboxylate

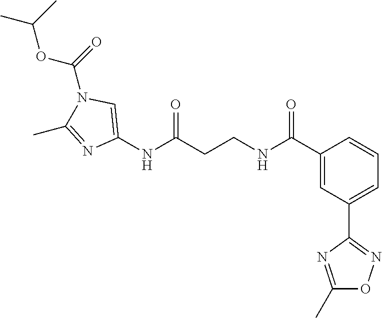 [SMILES: Cc1nc(-c2cccc(C(=O)NCCC(=O)Nc3cn(C(=O)OC(C)C)c(C)n3)c2)no1]

4.6 mg (4%) pale-yellow solid. HPLC/MS m/z: 441.05 [M+H]⁺, Rt (C): 0.77 min; ¹H NMR (400 MHz, DMSO-d₆): δ 10.53 (s, 1H), 8.79 (t, J=5.4 Hz, 1H), 8.46 (t, J=1.8 Hz, 1H), 8.16-8.09 (m, 1H), 8.07-7.99 (m, 1H), 7.65 (t, J=7.8 Hz, 1H), 7.46 (s, 1H), 5.12-5.04 (m, 1H), 3.57-3.51 (m, 2H), 2.69 (s, 3H), 2.62 (t, J=7.0 Hz, 2H), 2.49 (s, 3H), 1.35 (d, J=6.3 Hz, 6H).

### Example 265: Ethyl 2-methyl-4-(3-{[3-(5-methyl-1,2,4-oxadiazol-3-yl)phenyl]formamidolpropanamido)-1 H-imidazole-1-carboxylate

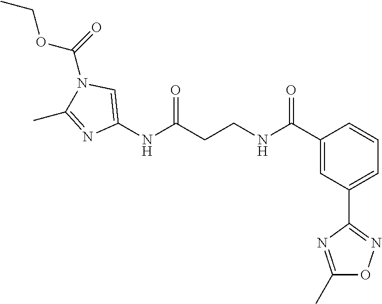 [SMILES: CCOC(=O)n1cc(NC(=O)CCNC(=O)c2cccc(-c3noc(C)n3)c2)nc1C]

16.3 mg (15%) colorless solid. HPLC/MS m/z: 427.10 [M+H]⁺, Rt (C): 0.72 min; ¹H NMR (400 MHz, DMSO-d₆): δ 10.54 (s, 1H), 8.80 (t, J=5.6 Hz, 1H), 8.47 (t, J=1.8 Hz, 1H), 8.16-8.09 (m, 1H), 8.07-8.00 (m, 1H), 7.66 (t, J=7.8 Hz, 1H), 7.48 (s, 1H), 4.42-4.32 (m, 2H), 3.60-3.50 (m, 2H), 2.69 (s, 3H), 2.63 (t, J=7.0 Hz, 2H), 2.50-2.47 (m, 3H), 1.34 (t, J=7.1 Hz, 3H).

### Example 266: Propyl 4-(3-{[3-(5-methyl-1,2,4-oxadiazol-3-yl)phenyl]formamido}-propanamido)-1 H-imidazole-1-carboxylate

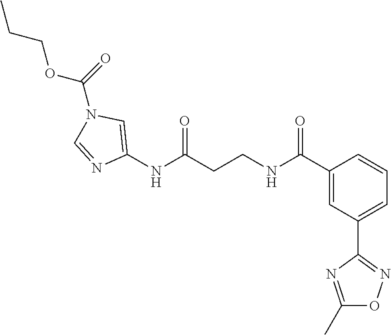 [SMILES: CCCOC(=O)n1cnc(NC(=O)CCNC(=O)c2cccc(-c3noc(C)n3)c2)c1]

13.3 mg (15%) off-white solid. HPLC/MS m/z: 427.20 [M+H]⁺, Rt (G): 1.32 min; ¹H NMR (300 MHz, DMSO-d₆): δ 10.70 (s, 1H), 8.83 (t, J=5.5 Hz, 1H), 8.47 (t, J=1.7 Hz, 1H), 8.19-8.08 (m, 2H), 8.04 (dt, J=7.9, 1.4 Hz, 1H), 7.65 (t, J=7.8 Hz, 1H), 7.56 (d, J=1.6 Hz, 1H), 4.32 (t, J=6.5 Hz, 2H), 3.57 (q, J=6.6 Hz, 2H), 2.67 (d, J=8.5 Hz, 5H), 1.74 (sext, J=7.1 Hz, 2H), 0.96 (t, J=7.4 Hz, 3H).

### Example 267: Propyl 4-[(2S)-2-hydroxy-3-{[3-(1-methyl-1 H-pyrazol-4-yl)phenyl]formamidolpropanamido]-2-methyl-1 H-imidazole-1-carboxylate

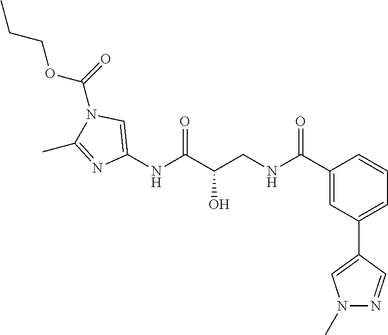 [SMILES: CCCOC(=O)n1cc(NC(=O)[C@@H](O)CNC(=O)c2cccc(-c3cnn(C)c3)c2)nc1C]

Example 267.1: A mixture of 2-methyl-4-nitro-1 H-imidazole (950.0 mg, 7.474 mmol) and sodium hydride (346.0 g, 8.650 mmol; 60%) in DMF (30.0 mL) was stirred for 30 min at rt. [2-(chloromethoxy)ethyl]trimethylsilane (1.39 g, 8.313 mmol) was added and the reaction mixture was stirred for 1 h at rt. The reaction was quenched by the addition of MeOH (5 mL) at 0 0° C. The resulting mixture was diluted with water, extracted with ethyl acetate. The combined organic layers were washed with brine, dried over anhydrous sodium sulfate, filtered, and concentrated under reduced pressure to give 1.64 g (85%) of 2-methyl-4-nitro-1-{[2-(trimethylsilyl)-ethoxy]methyl}-1 H-imidazole as an orange-brown solid. HPLC/MS m/z: 258.15 [M+H]⁺, Rt (A): 0.78 min.

Example 267.2: To a solution of 2-methyl-4-nitro-1-{[2-(trimethylsilyl)ethoxy]methyl}-1 H-imidazole (1.64 g, 6.372 mmol) in THF (20.0 mL) was added Pd/C (10%, 0.20 g; 0.188 mmol) under nitrogen atmosphere and the mixture was hydrogenated at rt for 16 h. The reactions mixture was filtered and the solid was washed with THF. The combined filtrates were evaporated, and the crude product (770 mg) was used in the next step without further purification. HPLC/MS m/z: 228.20 [M+H]⁺, Rt (A): 0.64 min.

Example 267.3: A solution of 3-{[(benzyloxy)carbonyl]amino}-2-hydroxypropanoic acid (4.00 g, 16.721 mmol) was treated with thionyl chloride (2.09 g, 17.712 mmol) in dichloromethane (40.0 mL) and stirred at 40° C. for 2 h. The reaction mixture was concentrated under reduced pressure and the residue was added to a stirred solution of 2-methyl-1-{[2-(trimethylsilyl)ethoxy]methyl}-1 H-imidazol-4-amine (450.0 mg, 1.979 mmol) and triethylamine (665.0 mg; 6.572 mmol) in THF (10.0 mL) at rt and stirred for 2 h. The reaction was quenched by the addition of methanol (5.0 mL) at 0° C., and the resulting mixture concentrated under reduced pressure. The residue was purified by silica gel column chromatography (eluent: methanol/dichloromethane—4:96) to afford 704.0 mg (79%) of benzyl N-{2-hydroxy-2-[(2-methyl-1-{[2-(trimethylsilyl)ethoxy]methyl}-1 H-imidazol-4-yl)carbamoyl]-ethyl}carbamate as a brown oil. HPLC/MS m/z: 449.25 [M+H]⁺, Rt (A): 0.66 min.

Example 267.4: To a solution of benzyl N-{2-hydroxy-2-[(2-methyl-1-{[2-(trimethyl-silyl)ethoxy]methyl}-1 H-imidazol-4-yl)carbamoyl]ethyl}carbamate (704.0 mg, 1.569 mmol) in THF (8.0 mL) was added Pd/C (80 mg) in a pressure tank. The mixture was hydrogenated at rt under 30 psi of hydrogen pressure for 2 h, filtered through a celite pad and concentrated under reduced pressure to give 469.0 mg (95%) of 3-amino-2-hydroxy-N-(2-methyl-1-{[2-(trimethylsilyl)ethoxy]methyl}-1 H-imidazol-4-yl)propanamide as a brown oil. HPLC/MS m/z: 315.20 [M+H]⁺, Rt (A): 0.53 min.

Example 267.5: To a stirred solution of 3-amino-2-hydroxy-N-(2-methyl-1-{[2-(trimethylsilyl)ethoxy]methyl}-1 H-imidazol-4-yl)propanamide (5.11 g, 16.250 mmol) and 3-(1-methyl-1 H-pyrazol-4-yl)benzoic acid (3.29 g, 16.270 mmol) in DMF (10.0 mL) were added 1-methyl-1 H-imidazole (3.33 g, 40.412 mmol) and TCFH (11.34 g, 40.416 mmol), and the reaction mixture was stirred at rt overnight. The mixture was concentrated under vacuum, and the crude product was purified, and the enantiomers separated by chiral SFC (column: CHIRAL ART Cellulose-SB, 4.6*100 mm, 3.0 μm; eluent: CO₂: methanol (containing 0.1% diethylamine)-90:10). Yield: 325.0 mg (4.0%) (2S)-2-hydroxy-N-(2-methyl-1-{[2-(trimethylsilyl)ethoxy]methyl}-1 H-imidazol-4-yl)-3-{[3-(1-methyl-1 H-pyrazol-4-yl)phenyl]formamido}propanamide as colorless solid. HPLC/MS m/z: 499.30 [M+H]⁺, Rt (A): 0.74 min. SFC: Rt: 1.62 min. 363.0 mg (4.5%) (2R)-2-hydroxy-N-(2-methyl-1-{[2-(trimethylsilyl)ethoxy]methyl}-1 H-imidazol-4-yl)-3-{[3-(1-methyl-1 H-pyrazol-4-yl)phenyl]formamidolpropanamide as off-white solid. HPLC/MS m/z: 499.30 [M+H]⁺, Rt (A): 0.74 min. SFC: Rt: 2.25 min.

Example 267.6: (2S)-2-hydroxy-N-(2-methyl-1-{[2-(trimethylsilyl)ethoxy]methyl}-1 H-imidazol-4-yl)-3-{[3-(1-methyl-1 H-pyrazol-4-yl)phenyl]formamido}propanamide (325.0 mg, 0.652 mmol) was treated with a solution of HCl in 1,4-dioxane (4 M, 5.0 mL) and stirred at 40° C. for 2 h. The resulting mixture was concentrated under reduced pressure, and the residue was purified via a strong cation exchange sorbent (ISOLUTE@SCX-2) to give 213.5 mg (89%) of (2S)-2-hydroxy-N-(2-methyl-1 H-imidazol-4-yl)-3-{[3-(1-methyl-1 H-pyrazol-4-yl)phenyl]formamidolpropanamide as a brown oil. HPLC/MS m/z: 369.20 [M+H]⁺, Rt (A): 0.56 min.

Example 267.7: To a stirred solution of (2S)-2-hydroxy-N-(2-methyl-1 H-imidazol-4-yl)-3-{[3-(1-methyl-1 H-pyrazol-4-yl)phenyl]formamido}propanamide (51.0 mg, 0.138 mmol) and triethylamine (45.6 mg, 0.451 mmol) in dichloromethane (9.0 mL) was added propyl carbonochloridate (14.2 mg, 0.116 mmol) dropwise at 00° C. The resulting mixture was slowly warmed to rt and stirred for 1 h. The reaction mixture was concentrated under reduced pressure, and the crude product was purified by prep-HPLC to afford 8.7 mg (14%) of propyl 4-[(2S)-2-hydroxy-3-{[3-(1-methyl-1 H-pyrazol-4-yl)phenyl]formamidolpropanamido]-2-methyl-1 H-imidazole-1-carboxylate as a colorless solid. HPLC/MS m/z: 455.20 [M+H]⁺, Rt (C): 0.82 min. ¹H NMR (300 MHz, DMSO-d₆): δ 9.97 (s, 1H), 8.50 (d, J=5.7 Hz, 1H), 8.16 (s, 1H), 8.00 (s, 1H), 7.87 (s, 1H), 7.72-7.59 (m, 2H), 7.51 (s, 1H), 7.41 (t, J=7.7 Hz, 1H), 5.93 (d, J=5.9 Hz, 1H), 4.28 (t, J=6.4 Hz, 3H), 3.86 (s, 3H), 3.61 (d, J=14.1 Hz, 1H), 3.50-3.37 (m, 1H), 2.47 (s, 3H), 1.80-1.65 (m, 2H), 0.94 (t, J=7.4 Hz, 3H).

Example 268: Propyl 4-[(2R)-2-hydroxy-3-{[3-(1-methyl-1 H-pyrazol-4-yl)phenyl]formamidolpropanamido]-2-methyl-1 H-imidazole-1-carboxylate

The R-enantiomer was prepared accordingly.
HPLC/MS m/z: 455.10 [M+H]⁺, Rt (C): 0.70 min. ¹H NMR (300 MHz, DMSO-d₆): δ 9.97 (s, 1H), 8.51 (t, J=5.7 Hz, 1H), 8.16 (s, 1H), 8.00 (d, J=1.9 Hz, 1H), 7.92-7.84 (m, 1H), 7.73-7.58 (m, 2H), 7.51 (s, 1H), 7.41 (t, J=7.7 Hz, 1H), 6.02-5.89 (m, 1H), 4.28 (t, J=6.5 Hz, 3H), 3.86 (s, 3H), 3.66-3.54 (m, 1H), 3.50-3.35 (m, 1H), 2.45 (s, 3H), 1.72 (sext, J=7.1 Hz, 2H), 0.94 (t, J=7.4 Hz, 3H).

The following examples were prepared analogously:

### Example 269: Propan-2-yl 4-[(2S)-2-hydroxy-3-{[3-(1-methyl-1 H-pyrazol-4-yl)phenyl]formamidol-propanamido]-2-methyl-1 H-imidazole-1-carboxylate

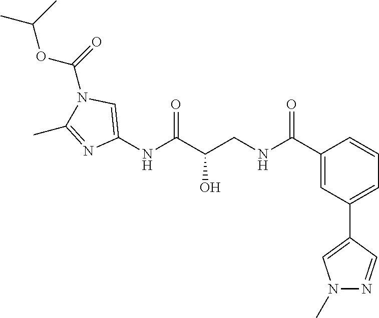 [SMILES: Cc1nc(NC(=O)[C@@H](O)CNC(=O)c2cccc(-c3cnn(C)c3)c2)cn1C(=O)OC(C)C]

9.5 mg (15%) colorless solid. HPLC/MS m/z: 455.20 [M+H]⁺, Rt (C): 0.82 min; ¹H NMR (300 MHz, DMSO-d₆): δ 9.95 (s, 1H), 8.51 (t, J=5.8 Hz, 1H), 8.16 (s, 1H), 8.00 (t, J=1.8 Hz, 1H), 7.88 (d, J=0.8 Hz, 1H), 7.71-7.60 (m, 2H), 7.48 (s, 1H), 7.41 (t, J=7.7 Hz, 1H), 5.15-5.00 (m, 1H), 4.34-4.23 (m, 1H), 3.86 (s, 4H), 3.65-3.58 (m, 1H), 3.45 (s, 1H), 2.45 (s, 3H), 1.34 (d, J=6.3 Hz, 6H).

### Example 270: Propan-2-yl 4-[(2R)-2-hydroxy-3-{[3-(1-methyl-1 H-pyrazol-4-yl)phenyl]formamidol-propanamido]-2-methyl-1 H-imidazole-1-carboxylate

HPLC/MS m/z: 455.10 [M+H]⁺, Rt (C): 0.70 min. ¹H NMR (300 MHz, DMSO-d₆): δ 9.95 (s, 1H), 8.51 (t, J=5.8 Hz, 1H), 8.16 (s, 1H), 8.05-7.97 (m, 1H), 7.89-7.84 (m, 1H), 7.73-7.51 (m, 2H), 7.52-7.35 (m, 2H), 6.02-5.90 (m, 1H), 5.16-4.98 (m, 1H), 4.35-4.23 (m, 1H), 3.86 (s, 3H), 3.68-3.54 (m, 1H), 3.50-3.34 (m, 1H), 2.49 (s, 3H), 1.34 (d, J=6.2 Hz, 6H).

### Example 271: Ethyl 4-[(2S)-2-hydroxy-3-{[3-(1-methyl-1 H-pyrazol-4-yl)phenyl]formamidolpropanamido]-2-methyl-1 H-imidazole-1-carboxylate

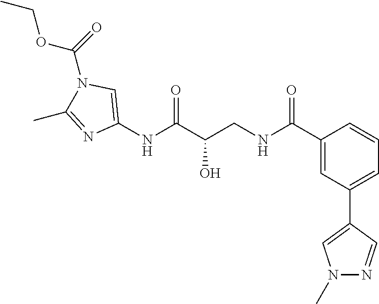 [SMILES: CCOC(=O)n1cc(NC(=O)[C@@H](O)CNC(=O)c2cccc(-c3cnn(C)c3)c2)nc1C]

HPLC/MS m/z: 441.05 [M+H]⁺, Rt (C): 0.65 min. ¹H NMR (300 MHz, DMSO-d₆): δ 9.96 (s, 1H), 8.50 (t, J=5.7 Hz, 1H), 8.16 (s, 1H), 8.00 (t, J=1.7 Hz, 1H), 7.88 (d, J=0.8 Hz, 1H), 7.73-7.58 (m, 2H), 7.50 (s, 1H), 7.41 (t, J=7.7 Hz, 1H), 5.93 (d, J=5.5 Hz, 1H), 4.36 (q, J=7.1 Hz, 2H), 4.28 (s, 1H), 3.86 (s, 3H), 3.68-3.54 (m, 1H), 3.50-3.35 (m, 2H), 2.51 (s, 3H), 1.32 (t, J=7.1 Hz, 3H).

### Example 272: tert-Butyl 4-(3-{[7-(5-methyl-1,2,4-oxadiazol-3-yl)isoquinolin-1-yl]amino}propanamido)-1 H-pyrazole-1-carboxylate

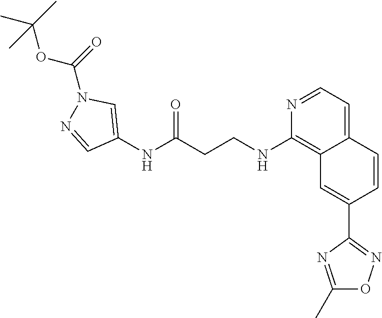 [SMILES: Cc1nc(-c2ccc3ccnc(NCCC(=O)Nc4cnn(C(=O)OC(C)(C)C)c4)c3c2)no1]

Example 272.1: 1-Chloro-7-(5-methyl-1,2,4-oxadiazol-3-yl)isoquinoline [Example 45](243.0 mg, 0.989 mmol) and tert-butyl 3-aminopropanoate (861.8 mg, 5.935 mmol) were dissolved in NMP (17.0 mL) in a microwave vessel. N-Ethyldiiso-propylamine (420.5 μl, 2.473 mmol) was added and the mixture was stirred at 150° C. for 20 h. The reaction mixture was cooled to rt, diluted with water, and extracted with ethyl acetate. The combined organic layers were washed with brine, dried over sodium sulfate, filtered, and concentrated in vacuo. The residue was purified by RP-flash chromatography, the product fractions were combined, diluted with saturated aqueous NaHCO₃-solution and extracted with ethyl acetate. The combined organic layers were washed with brine, dried over sodium sulfate, filtered, and concentrated in vacuo to give 192 mg (55%) of tert-butyl 3-{[7-(5-methyl-1,2,4-oxadiazol-3-yl)isoquinolin-1-yl]amino}propanoate as an orange solid. HPLC/MS m/z: 354.9 [M+H]⁺, Rt (F): 0.74 min.

Example 272.2: To a solution of tert-butyl 3-{[7-(5-methyl-1,2,4-oxadiazol-3-yl)isoquinolin-1-yl]amino}propanoate (192.0 mg, 0.542 mmol) in dichloromethane (3.0 mL) trifluoroacetic acid (788.5 μl, 10.235 mmol) was added and the yellow solution was stirred at rt overnight. The reaction mixture was concentrated under vacuum and the residue (165 mg) was used in the next step without further purification. HPLC/MS m/z: 289.9 [M+H]⁺, Rt (F): 0.65 min.

Example 272.3: 3-{[7-(5-methyl-1,2,4-oxadiazol-3-yl)isoquinolin-1-yl]amino}propanoic acid (162.0 mg, 0.543 mmol) and tert-butyl 4-amino-1 H-pyrazole-1-carboxylate (99.5 mg, 0.543 mmol) were dissolved in DMF (2.0 mL). [Dimethylamino-([1,2,3]triazolo[4,5-b]pyridin-3-yloxy)-methylene]-dimethyl-ammonium; hexafluoro phosphate (268.1 mg, 0.705 mmol) and subsequently N-Ethyldiisopropylamine (372.8 μl, 2.170 mmol) were added and the mixture was stirred at rt overnight. The reaction mixture was diluted with ethyl acetate and extracted with water and saturated aqueous NaHCO₃-solution. The combined aqueous phase was extracted with ethyl acetate, the combined organic layers were washed with brine, dried over sodium sulfate, filtered, and evaporated to dryness. The product mixture was separated by RP-flash chromatography and the products were freeze-dried. Yield: 4.4 mg (2%) of tert-butyl 4-(3-{[7-(5-methyl-1,2,4-oxadiazol-3-yl)isoquinolin-1-yl]amino}propanamido)-1 H-pyrazole-1-carboxylate, colorless solid. HPLC/MS m/z: 463.8 [M+H]⁺, Rt (F): 0.74 min. ¹H NMR (700 MHz, DMSO-d₆): δ 10.29-10.26 (m, 1H), 8.90-8.86 (m, 1H), 8.33 (s, 1H), 8.17-8.13 (m, 1H), 8.03-7.96 (m, 2H), 7.87-7.83 (m, 1H), 7.76 (s, 1H), 7.00-6.95 (m, 1H), 3.78 (q, J=6.5 Hz, 2H), 2.73 (t, J=7.0 Hz, 2H), 2.70 (s, 3H), 1.57 (s, 9H). Also 46 mg (23%) of 3-{[7-(5-methyl-1,2,4-oxadiazol-3-yl)isoquinolin-1-yl]amino}-N-(1 H-pyrazol-4-yl)propenamide were isolated as a colorless solid. HPLC/MS m/z: 363.9 [M+H]⁺, Rt (F): 0.65 min.

### Example 273: Propyl 4-(3-{[7-(5-methyl-1,2,4-oxadiazol-3-yl)isoquinolin-1-yl]amino}propanamido)-1 H-pyrazole-1-carboxylate

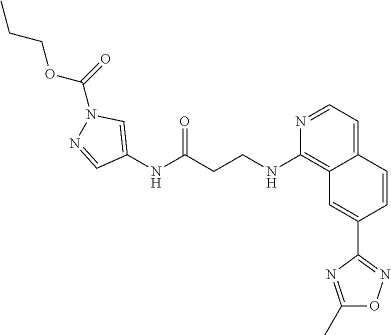 [SMILES: CCCOC(=O)n1cc(NC(=O)CCNc2nccc3ccc(-c4noc(C)n4)cc23)cn1]

3-{[7-(5-methyl-1,2,4-oxadiazol-3-yl)isoquinolin-1-yl]amino}-N-(1 H-pyrazol-4-yl)propanamide (46.0 mg, 0.127 mmol) was suspended in dry dichloromethane (0.9 mL) and treated with dry pyridine (1.15 mL, 14.305 mmol). To this almost clear solution propyl carbonochloridate (1M in dry THF, 126.6 μl, 0.127 mmol) and further dry THF (1.16 mL) were added, and the reaction was stirred at rt for 5 h. Further propyl carbonochloridate (1 M in dry THF, 38.0 μl, 0.038 mmol) was added and the reaction was stirred at rt for 3 h. The reaction mixture was diluted with water and extracted with ethyl acetate. The combined organic layers were washed with brine, dried over sodium sulfate, filtered, and concentrated in vacuo. The residue was purified by RP-flash chromatography. Yield: 16 mg (28%) of the title compound as a colorless solid. HPLC/MS m/z: 449.8 [M+H]⁺, Rt (F): 0.72 min. ¹H NMR (500 MHz, DMSO-d₆): δ 10.30 (s, 1H), 8.89-8.87 (m, 1H), 8.39-8.38 (m, 1H), 8.15 (dd, J=8.5, 1.5 Hz, 1H), 8.02-7.97 (m, 1H), 7.99 (d, J=5.7 Hz, 1H), 7.85 (d, J=8.5 Hz, 1H), 7.80 (d, J=0.7 Hz, 1H), 6.97 (d, J=5.7 Hz, 1H), 4.33 (t, J=6.6 Hz, 2H), 3.81-3.76 (m, 2H), 2.74 (t, J=6.9 Hz, 2H), 2.70 (s, 3H), 1.78-1.70 (m, 2H), 0.95 (t, J=7.4 Hz, 3H).

The following examples were prepared in a similar manner:

### Example 274: tert-Butyl 4-(3-{[3-(5-methyl-1,2,4-oxadiazol-3-yl)phenyl]formamido}-propanamido)-1 H-pyrazole-1-carboxylate

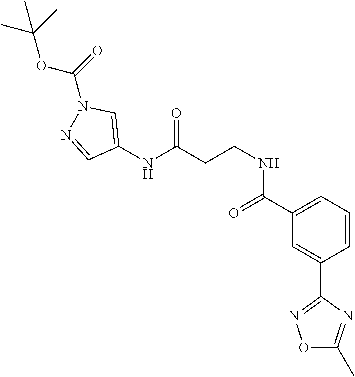 [SMILES: Cc1nc(-c2cccc(C(=O)NCCC(=O)Nc3cnn(C(=O)OC(C)(C)C)c3)c2)no1]

3-{[3-(5-methyl-1,2,4-oxadiazol-3-yl)phenyl]formamido}propanoic acid (50.0 mg, 0.182 mmol) and tert-butyl 4-amino-1 H-pyrazole-1-carboxylate (33.3 mg, 0.182 mmol) were dissolved in DMF (0.6 mL). [Dimethylamino-([1,2,3]triazolo[4,5-b]pyridin-3-yloxy)-methylene]-dimethyl-ammonium; hexafluoro phosphate (69.2 mg; 0.182 mmol) and N-Ethyldiisopropylamine (124.8 μl, 0.727 mmol) were added and the reaction mixture was stirred at rt overnight. The reaction mixture was evaporated, and the residue purified by RP-flash chromatography to give 59 mg (73%) of the title compound as a colorless solid. HPLC/MS m/z: 340.9 [M+H-Boc]+, Rt (F): 0.88 min. ¹H NMR (500 MHz, DMSO-d₆): δ 10.32 (s, 1H), 8.83 (t, J=5.5 Hz, 1H), 8.46 (t, J=1.8 Hz, 1H), 8.32 (d, J=0.7 Hz, 1H), 8.14-8.11 (m, 1H), 8.04-8.02 (m, 1H), 7.76 (d, J=0.7 Hz, 1H), 7.65 (t, J=7.8 Hz, 1H), 3.60-3.54 (m, 2H), 2.68 (s, 3H), 2.62 (t, J=7.0 Hz, 2H), 1.57 (s, 9H).

### Example 275: Propyl 4-(3-{[3-(5-methyl-1,2,4-oxadiazol-3-yl)phenyl]formamido}-propanamido)-1 H-pyrazole-1-carboxylate

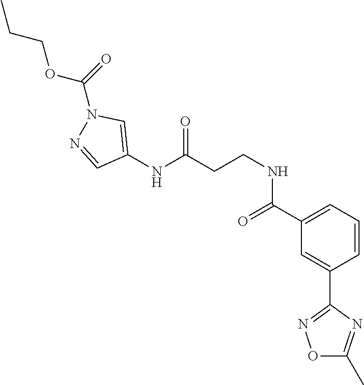 [SMILES: CCCOC(=O)n1cc(NC(=O)CCNC(=O)c2cccc(-c3noc(C)n3)c2)cn1]

41 mg (45%) colorless solid. HPLC/MS m/z: 426.8 [M+H-Boc]⁺, Rt (F): 0.86 min. ¹H NMR (700 MHz, DMSO-d₆): δ 10.40 (s, 1H), 8.86 (t, J=5.5 Hz, 1H), 8.47-8.46 (m, 1H), 8.39-8.37 (m, 1H), 8.13-8.11 (m, 1H), 8.04-8.02 (m, 1H), 7.82-7.81 (m, 1H), 7.65 (t, J=7.7 Hz, 1H), 4.33 (t, J=6.6 Hz, 2H), 3.59-3.55 (m, 2H), 2.68 (s, 3H), 2.63 (t, J=7.0 Hz, 2H), 1.77-1.71 (m, 2H), 0.95 (t, J=7.4 Hz, 3H).

### Example 276: Propyl 4-methyl-2-(3-{[6-(5-methyl-1,2,4-oxadiazol-3-yl)pyridin-2-yl]formamido}propanamido)-1,3-thiazole-5-carboxylate

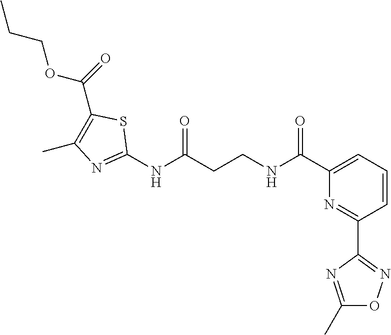 [SMILES: CCCOC(=O)c1sc(NC(=O)CCNC(=O)c2cccc(-c3noc(C)n3)n2)nc1C]

Example 276.1: 6-Cyanopyridine-2-carboxylic acid (500.0 mg, 3.376 mmol), hydroxylammonium chloride (258.0 mg, 3.713 mmol), sodium carbonate (715.6 mg, 6.751 mmol) and 8-hydroxyquinoline (24.5 mg, 0.169 mmol) were suspended in ethanol (20.0 mL) under argon atmosphere. The mixture was heated to 50° C. and stirred for 3 h. The reaction mixture was cooled to rt and evaporated to dryness. The residue (1.35 g light brown solid; HPLC/MS m/z: 182.1 [M+H]⁺, Rt (M): 0.69 min), which was used in the next step without further purification, was suspended in glacial acetic acid (5.0 mL) and acetic anhydride (0.75 mL, 7.935 mmol), and the mixture was refluxed for 3 h, while a brown suspension was formed. The reaction was cooled to rt and evaporated to dryness. The solid residue was purified by flash-chromatography to yield 531 mg (77%) of 6-(5-methyl-1,2,4-oxadiazol-3-yl)pyridine-2-carboxylic acid as a red-brown solid. HPLC/MS m/z: 206.0 [M+H]⁺, Rt (M): 1.10 min.

Example 276.2: Prepared as described for Example 1 using intermediate 276.1. Yield: 67 mg (57%) as colorless solid. HPLC/MS m/z: 459.1 [M+H]⁺, Rt (H): 1.51 min. ¹H NMR (400 MHz, DMSO-d₆): δ 12.52 (s, 1H), 8.69 (t, J=6.1 Hz, 1H), 8.25-8.19 (m, 3H), 4.16 (t, J=6.5 Hz, 2H), 3.68 (q, J=6.6 Hz, 2H), 2.81 (t, J=6.8 Hz, 2H), 2.71 (s, 3H), 2.53 (s, 3H), 1.73-1.63 (m, 2H), 0.95 (t, J=7.4 Hz, 3H).

### Example 277: tert-Butyl 4-methyl-2-(3-{[6-(5-methyl-1,2,4-oxadiazol-3-yl)pyridin-2-yl]formamido}propanamido)-1,3-thiazole-5-carboxylate

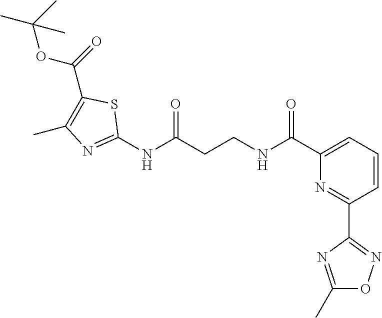 [SMILES: Cc1nc(-c2cccc(C(=O)NCCC(=O)Nc3nc(C)c(C(=O)OC(C)(C)C)s3)n2)no1]

7 mg (33%) pale-brown solid. HPLC/MS m/z: 473.1 [M+H]⁺, Rt (H): 1.57 min. ¹H NMR (700 MHz, DMSO-d₆): δ 12.47 (s, 1H), 8.69 (t, J=6.1 Hz, 1H), 8.24 (dd, J=6.9, 2.3 Hz, 1H), 8.21 (d, J=5.8 Hz, 1H), 8.23-8.19 (m, 1H), 3.67 (q, J=6.6 Hz, 2H), 2.80 (t, J=6.8 Hz, 2H), 2.72 (s, 3H), 2.50 (s, 3H), 1.51 (s, 9H).

### Example 278: Propan-2-yl 4-methyl-2-(3-{[6-(5-methyl-1,2,4-oxadiazol-3-yl)pyridin-2-yl]formamidolpropanamido)-1,3-thiazole-5-carboxylate

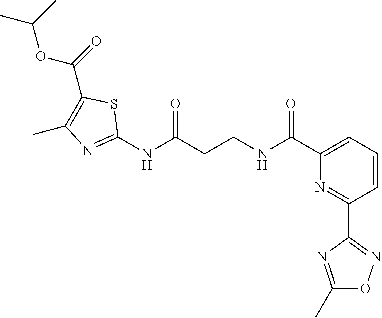 [SMILES: Cc1nc(-c2cccc(C(=O)NCCC(=O)Nc3nc(C)c(C(=O)OC(C)C)s3)n2)no1]

62 mg (58%) colorless solid. HPLC/MS m/z: 459.1 [M+H]⁺, Rt (H): 1.50 min. ¹H NMR (400 MHz, DMSO-d₆): δ 12.52-12.47 (m, 1H), 8.68 (t, J=6.2 Hz, 1H), 8.25-8.18 (m, 3H), 5.11-5.00 (m, 1H), 3.68 (q, J=6.6 Hz, 2H), 2.81 (t, J=6.8 Hz, 2H), 2.71 (s, 3H), 2.53 (s, 3H), 1.28 (d, J=6.2 Hz, 6H).

### Example 279: tert-Butyl 2-(3-{[2-fluoro-3-(5-methyl-1,2,4-oxadiazol-3-yl)phenyl]formamidolpropanamido)-4-methyl-1,3-thiazole-5-carboxylate

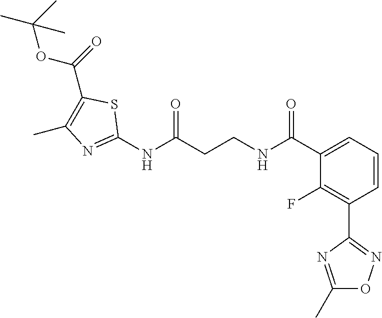 [SMILES: Cc1nc(-c2cccc(C(=O)NCCC(=O)Nc3nc(C)c(C(=O)OC(C)(C)C)s3)c2F)no1]

25 mg (19%) colorless solid. HPLC/MS m/z: 489.8 [M+H]⁺, Rt (H): 1.64 min. ¹H NMR (700 MHz, DMSO-d₆): δ 12.45 (s, 1H), 8.66 (t, J=5.6 Hz, 1H), 8.09-8.06 (m, 1H), 7.77-7.74 (m, 1H), 7.45 (t, J=7.7 Hz, 1H), 3.58 (q, J=6.5 Hz, 2H), 2.76 (t, J=6.8 Hz, 2H), 2.69 (s, 3H), 2.51 (s, 3H), 1.51 (s, 9H).

### Example 280: Propyl 4-methyl-2-[3-[(7-methyl-1-isoquinolyl)amino]propanoyl-amino]thiazole-5-carboxylate

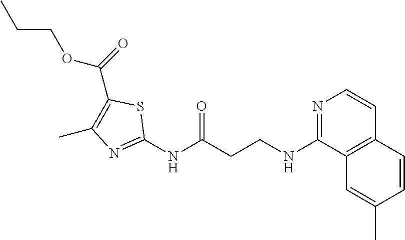 [SMILES: CCCOC(=O)c1sc(NC(=O)CCNc2nccc3ccc(C)cc23)nc1C]

Example 280.1: Tris(dibenzylideneacetone)dipalladium(0) (16.50 mg, 0.0180 mmol), 2-(dicyclohexylphosphino)-3,6-dimethoxy-2′-4′-6′-tri-1-propyl-1,1′-biphenyl (20.15 mg, 0.0360 mmol), (3-tert-butoxy-3-oxo-propyl)ammonium chloride (122.72 mg, 0.6756 mmol), sodium tert-butoxide (129.85 mg, 1.3511 mmol) and 1-chloro-7-methyl-isoquinoline (80.00 mg, 0.4504 mmol) were added to a microwave vial. The vial was sealed under nitrogen atmosphere and toluene (2.77 mL) was added. Nitrogen was bubbled through the solution under vigorous stirring for 20 min and the solution was heated at 150° C. for 20 min. The solution was cooled down to rt and volatiles were removed under reduced pressure to afford 294.4 mg of crude product. Purification by silica gel column chromatography (eluent: 0-20% EtOAc in DCM) and then further silica gel column chromatography (eluent: 0-15% MeOH in DCM) to give 3-[(7-methyl-1-isoquinolyl)amino]propanoic acid (40.3 mg, 39%, 0.1750 mmol) that was used in the next step without further purification. HPLC/MS m/z: 231.11, [M+H]⁺, Rt (P): 0.84 min.

Example 280.2: 3-[(7-methyl-1-isoquinolyl)amino]propanoic acid and propyl 2-amino-4-methyl-thiazole-5-carboxylate were used in an analogous procedure to Example 45.7 to afford Propyl 4-methyl-2-[3-[(7-methyl-1-isoquinolyl)amino]-propanoylamino]thiazole-5-carboxylate (30 mg, 42%) as a colorless, amorphous solid. HPLC/MS m/z: 413.26 [M+H]⁺, Rt (T): 1.27 min. ¹H NMR (600 MHz, DMSO-d₆) 6 12.58 (s, 1H), 7.99 (s, 1H), 7.80 (d, J=5.8 Hz, 1H), 7.60 (d, J=8.2 Hz, 1H), 7.46 (dd, J=8.4, 1.6 Hz, 1H), 7.42 (t, J=5.5 Hz, 1H), 6.86 (d, J=5.8 Hz, 1H), 4.16 (t, J=6.5 Hz, 2H), 3.78 (q, J=6.4 Hz, 2H), 2.87 (t, J=6.8 Hz, 2H), 2.54 (s, 3H), 2.45 (s, 3H), 1.68 (q, J=7.0 Hz, 2H), 0.94 (t, J=7.4 Hz, 3H).

### Example 281: Testing compounds of the present invention for inhibitory activities against HSET—HSET ADP-Glo Assay S—3 μM ATP

Reagents: (+4° C. Storage)
Paclitaxel Prod.No.TXD.01 2 mM in DMSO (from Universal Biologicals Cambridge).
Tubulin Protein (Pre-formed Microtubules): Bovine Brain Prod.No.MT001-XL Lot.025 10 mg/mL (from Universal Biologicals Cambridge).

Reagents: (−80° C. Storage)
MT001-XL—Tubulin Protein (Pre-formed Microtubules): Bovine Brain—reconstituted in buffer—15 mM PIPES pH7, 1 mM MgCl2, 20 μM paclitaxel (aliquots at 10 mg/mL) from Universal Biologicals Cambridge.
HSET full length protein—Current batch is FL HSET Prep1 (SEQ-000096_002-01_01)
Buffer is 20 mM Hepes pH 7.5, 200 mM NaCl, 2 mM TCEP, 5% glycerol (5 μL aliquots at 10.2 μM)

Reagents: (−20° C. Storage)
ADP-Glo Kinase Assay kit (Promega Prod.No.V9102) 10,000 assay points
ADP-Glo reagent (50 mL), Kinase Detection Reagent (100 mL), and Ultrapure ATP (10 mM) aliquoted

Buffer Stocks (filtered and stored at rt for up to 1 month)
HEPES acid, 4-(2-Hydroxyethyl)piperazine-1-ethanesulfonic acid MW:238, 238.3 mg/mL=1 M
7149 mg/30 mL=1M (pH to 6.8 with 5 M NaOH)
PIPES, 1,4-Piperazinediethanesulfonic acid MW: 302.4, 30.24 mg/mL=100 mM; 907.2 mg/30 mL=100 mM (pH to 7 with 5 M NaOH, white cloudy suspension until the pH changes)
EGTA, Ethylene glycol-bis(2-aminoethylether)-N,N,N′,N′-tetraacetic acid MW: 380.35, 38.035 mg/mL=100 mM
1901.75 mg/50 mL=100 mM (pH to ˜7 with 5 M NaOH)—takes a long time to get into solution and for pH to stabilize
Triton-X-100, MW: 625, 62.5 mg/mL=100 mM (6.25% w/v) (viscous, pipette Triton X-100 with a Gilson using a trimmed pipette tip)

ECHO Protocol
Create an ECHO intermediate plate by adding 24.5 μL DMSO to columns 1 & 2, and 40 μL DMSO to columns 23 & 24 of an ECHO 384PP plate
Add 100 nL of compound/DMSO per well in 384-well Proxi-Plate Plus (white) —Perkin Elmer Cat #6008289 using ECHO dose response protocol 100 nL normal to proxi_8pt_200 uM.

Assay buffer (Keep on ice): HEPES pH 6.8, MgCl₂, EGTA, Triton X-100, DTT, HPLC H₂O Microtubule Working Solution (3.2):
1 ml PM buffer: PIPES pH 7.0, MgCl₂, HPLC H₂O, Paclitaxel, mix well, store at rt
Then add 26.4 μL of 10 mg/ml microtubules to 724 μL of the above PM buffer to make 750 μL of microtubule working solution @350 μg/mL microtubules (store at rt)

Stock HSET enzyme solution (3.1)—keep on ice
Add 1.33 μL 10.2 μM HSET Prepi (SEQ-000096_002-01_01) to 1358 μL assay buffer to make a 10 nM stock for a 5 nM final assay concentration (1/1020 dilution).

HSET/Microtubule working solution (3.3)
Mix solutions 3.1 and 3.2 in the ratio of 2.5:1 (1214.3 μL 3.1+485.7 μL 3.2) keep at rt for 15 min
3.2 is diluted 3.5-fold, 3.1 is diluted 1.4-fold BLANK solution is 357 μL 2XAB+143 μL microtubule working solution 3.2. (Same proportions as HSET/Microtubule working solution (3.3))

ATP Working solution is 1 μL 10 mM UltraPure ATP (Promega kit)+999 μL ddH₂0 gives 10 μM ATP for a 3 μM final assay concentration, stored on ice.

Assay procedure in PROXIPLATE 384 PLUS WHITE (Perkin Elmer) plates (Remove 2.4 mL ADP Glo and 4.5 mL Kinase detection reagent from freezer to warm up to rt)
Add 3.5 μL BLANK solution to assay plate (column 12)
Add 3.5 μL HSET/Microtubule solution (3.3) (columns 1-11 & 13-24)
Centrifuge at 1000 rpm for 1 min
(pre-incubate enzyme and compound for 10 min)

Add 1.5 μL of 10 μM ATP to start reaction gives a final [HSET] of 5 nM, [ATP] of 3 μM and [microtubule] of 70 μg/mL, centrifuge at 1000 rpm for 1 min and incubated at rt for 80 min.

After the 80-minute incubation, stop reaction by adding 5 μL ADP-Glo reagent to all wells. Centrifuge for 1 min at 1000 rpm, leave for 40 min at rt.

In the dark/away from direct light, add 10 μL Kinase Detection Reagent (KDR) to all wells, Seal plate with a Topseal (Perkin Elmer Cat #6050185) and centrifuge as above, leave for 40 min at rt covered in foil.

Read luminescence on New Envision using Protocol: US luminescence 384.

### Results:

| Compound | COMBINED HSET |
|---|---|
| Example No. | ADP GLO IC₅₀ (μM) |
| 1 | 2.726 |
| 2 | 3.967 |
| 3 | 4.567 |
| 4 | 0.063 |
| 5 | 1.661 |
| 6 | 0.614 |
| 7 | 15.568 |
| 8 | 1.214 |
| 9 | 0.397 |
| 10 | 0.036 |
| 11 | 1.827 |
| 12 | 1.096 |
| 13 | 0.009 |
| 14 | 0.325 |
| 15 | 0.009 |
| 16 | 0.301 |
| 17 | 0.869 |
| 18 | 0.014 |
| 19 | 0.017 |
| 20 | 0.737 |
| 21 | 17.140 |
| 22 | 6.347 |
| 23 | 0.146 |
| 24 | 0.055 |
| 25 | 0.013 |
| 26 | 0.013 |
| 27 | 0.116 |
| 28 | 2.236 |
| 29 | 0.077 |
| 30 | 0.186 |
| 31 | 0.191 |
| 32 | 0.011 |
| 33 | 0.161 |
| 34 | 0.101 |
| 35 | 0.031 |
| 36 | 2.097 |
| 37 | 0.260 |
| 38 | 0.027 |
| 39 | 0.008 |
| 40 | 0.010 |
| 41 | 0.184 |
| 42 | 1.789 |
| 43 | 0.044 |
| 44 | 0.281 |
| 45 | 0.005 |
| 46 | 0.037 |
| 47 | 0.147 |
| 48 | 0.068 |
| 49 | 0.059 |
| 50 | 0.199 |
| 51 | 1.600 |
| 52 | 0.152 |
| 53 | 0.240 |
| 54 | 0.003 |
| 55 | 1.131 |
| 56 | 1.260 |
| 57 | 0.166 |
| 58 | 0.014 |
| 59 | 0.278 |
| 60 | 0.054 |
| 61 | 0.819 |
| 62 | 1.206 |
| 63 | 0.011 |
| 64 | 0.348 |
| 65 | 4.102 |
| 66 | 0.040 |
| 67 | 0.419 |
| 68 | 0.033 |
| 69 | 0.050 |
| 70 | 0.011 |
| 71 | 10.943 |
| 72 | 4.312 |
| 73 | 3.894 |
| 74 | 0.272 |
| 75 | 0.078 |
| 76 | 8.308 |
| 77 | 0.556 |
| 78 | 1.497 |
| 79 | 4.337 |
| 80 | 0.201 |
| 81 | 0.021 |
| 82 | 1.476 |
| 83 | 1.675 |
| 84 | 0.057 |
| 85 | 1.739 |
| 86 | 0.848 |
| 87 | 9.385 |
| 88 | 0.477 |
| 89 | 0.391 |
| 90 | 0.556 |
| 91 | 0.415 |
| 92 | 0.045 |
| 93 | 0.691 |
| 94 | 13.436 |
| 95 | 0.823 |
| 96 | 2.190 |
| 97 | 1.152 |
| 98 | 0.072 |
| 99 | 0.007 |
| 100 | 0.836 |
| 101 | 0.276 |
| 102 | 0.409 |
| 103 | 0.676 |
| 104 | 0.093 |
| 105 | 1.591 |
| 106 | 0.132 |
| 107 | 0.041 |
| 108 | 1.623 |
| 109 | 1.778 |
| 110 | 0.008 |
| 111 | 0.068 |
| 112 | 0.054 |
| 113 | 1.381 |
| 114 | 0.017 |
| 115 | 0.005 |
| 116 | 0.144 |
| 117 | 9.491 |
| 118 | 8.411 |
| 119 | 0.067 |
| 120 | 0.157 |
| 121 | 0.036 |
| 122 | 0.027 |
| 123 | 0.009 |
| 124 | 0.014 |
| 125 | 5.590 |
| 126 | 0.039 |
| 127 | 0.055 |
| 128 | 14.145 |
| 129 | 1.133 |
| 130 | 15.354 |
| 131 | 0.016 |
| 132 | 0.010 |
| 133 | 0.093 |
| 134 | 1.312 |
| 135 | 0.428 |
| 136 | 0.737 |
| 137 | 0.778 |
| 138 | 0.011 |
| 139 | 2.160 |
| 140 | 5.045 |
| 141 | 0.405 |
| 142 | 0.288 |
| 143 | 0.061 |
| 144 | 9.203 |
| 145 | 5.479 |
| 146 | 0.260 |
| 147 | 0.291 |
| 148 | 0.133 |
| 149 | 2.795 |
| 150 | 0.057 |
| 151 | 0.013 |
| 152 | 0.019 |
| 153 | 0.285 |
| 154 | 0.006 |
| 155 | 0.011 |
| 156 | 0.053 |
| 157 | 0.373 |
| 158 | 0.011 |
| 159 | 0.021 |
| 160 | 0.054 |
| 161 | 2.145 |
| 162 | 0.558 |
| 163 | 0.013 |
| 164 | 0.116 |
| 165 | 0.023 |
| 166 | 0.011 |
| 167 | 0.010 |
| 168 | 0.003 |
| 169 | 0.265 |
| 170 | 0.004 |
| 171 | 0.149 |
| 172 | 0.006 |
| 173 | 0.063 |
| 174 | 0.008 |
| 175 | 0.335 |
| 176 | 0.001 |
| 177 | 0.002 |
| 178 | 0.002 |
| 179 | 0.043 |
| 180 | 0.002 |
| 181 | 0.014 |
| 182 | 0.057 |
| 183 | 17.333 |
| 184 | 0.034 |
| 185 | 0.020 |
| 186 | 0.030 |
| 187 | 0.052 |
| 188 | 0.270 |
| 189 | 0.616 |
| 190 | 0.726 |
| 191 | 0.284 |
| 192 | 0.461 |
| 193 | 0.005 |
| 194 | 0.096 |
| 195 | 0.012 |
| 196 | 0.167 |
| 197 | 0.135 |
| 198 | 0.181 |
| 199 | 0.010 |
| 200 | 0.295 |
| 201 | 0.410 |
| 202 | 8.850 |
| 203 | 1.100 |
| 204 | 5.700 |
| 205 | 4.400 |
| 206 | 9.300 |
| 207 | 0.590 |
| 208 | 0.079 |
| 209 | 4.000 |
| 210 | 0.700 |
| 211 | 1.300 |
| 212 | 3.350 |
| 213 | 1.700 |
| 214 | 2.400 |
| 215 | 0.084 |
| 216 | 1.700 |
| 217 | 7.300 |
| 218 | 0.041 |
| 219 | 0.089 |
| 220 | 0.122 |
| 221 | 0.133 |
| 222 | 0.010 |
| 223 | 0.219 |
| 224 | 0.270 |
| 225 | 1.664 |
| 226 | 0.012 |
| 227 | 0.088 |
| 228 | 0.086 |
| 229 | 0.052 |
| 230 | 0.002 |
| 231 | 0.005 |
| 232 | 0.044 |
| 233 | 0.030 |
| 234 | 2.099 |
| 235 | 6.783 |
| 236 | 2.839 |
| 237 | 0.100 |
| 238 | 6.738 |
| 239 | 0.119 |
| 240 | 0.098 |
| 241 | 3.084 |
| 242 | 0.030 |
| 243 | 0.011 |
| 244 | 0.608 |
| 245 | 7.473 |
| 246 | 1.744 |
| 247 | 13.561 |
| 248 | 4.143 |
| 249 | 14.460 |
| 250 | 9.456 |
| 251 | 5.623 |
| 252 | 16.422 |
| 253 | 3.411 |
| 254 | 6.100 |
| 255 | 0.027 |
| 256 | 0.020 |
| 257 | 0.047 |
| 258 | 0.207 |
| 259 | 0.531 |
| 260 | 0.140 |
| 261 | 13.000 |
| 262 | 1.000 |
| 263 | 0.200 |
| 264 | 0.280 |
| 265 | 2.250 |
| 266 | 3.400 |
| 267 | 0.380 |
| 268 | 0.950 |
| 269 | 0.910 |
| 270 | 5.100 |
| 271 | 4.000 |
| 272 | 0.160 |
| 273 | 0.080 |
| 274 | 6.400 |
| 275 | 11.500 |
| 276 | 0.037 |
| 277 | 0.155 |
| 278 | 0.071 |
| 279 | 0.860 |
| 280 | 0.141 |

### Example 282: Injection vials

A solution of 100 g of a compound of the present invention and 5 g of disodium hydrogenphosphate in 3 L of bidistilled water is adjusted to pH 6.5 using 2 N hydrochloric acid, filtered under sterile conditions, transferred into injection vials, lyophilised under sterile conditions and sealed under sterile conditions. Each injection vial contains 5 mg of a compound of the present invention.

### Example 283: Solution

A solution is prepared from 1 g of a compound of the present invention, 9.38 g of NaH₂PO₄. 2 H₂O, 28.48 g of Na₂HPO₄. 12 H₂O and 0.1 g of benzalkonium chloride in 940 mL of bidistilled water. The pH is adjusted to 6.8, and the solution is made up to 1 L and sterilised by irradiation.

### Example 284: Ampoules

A solution of 1 kg of a compound of the present invention in 60 L of bidistilled water is filtered under sterile conditions, transferred into ampoules, lyophilised under sterile conditions and sealed under sterile conditions. Each ampoule contains 10 mg of a compound of the present invention.

### REFERENCES

Basto, R., Brunk, K., Vinadogrova, T., Peel, N., Franz, A., Khodjakov, A., and Raff, J. W. (2008). Centrosome amplification can initiate tumorigenesis in flies. Cell 133, 1032-1042.
Bender, A., Young, D. W., Jenkins, J. L., Serrano, M., Mikhailov, D., Clemons, P. A., and Davies, J. W. (2007). Chemogenomic data analysis: prediction of small-molecule targets and the advent of biological fingerprint. Comb. Chem. High Throughput Screen. 10, 719-731.
Bickerton, G. R., Paolini, G. V., Besnard, J., Muresan, S., and Hopkins, A. L. (2012). Quantifying the chemical beauty of drugs. Nat. Chem. 4, 90-98.
Boveri, T. (2008). Concerning the origin of malignant tumours by Theodor Boveri. Translated and annotated by Henry Harris. J. Cell Sci. 121(Suppl 1), 1-84.
Cai, S., Weaver, L. N., Ems-McClung, S. C., and Walczak, C. E. (2010). Proper organization of microtubule minus ends is needed for midzone stability and cytokinesis. Curr. Biol. 20, 880-885.
Cai, S., Weaver, L. N., Ems-McClung, S. C., Walczak, C. E. (2009) Kinesin-14 family proteins HSET/XCTK2 control spindle length by cross-linking and sliding microtubules. Molecular Biology of the Cell 20(5), pp. 1348-1359
Chan, J. Y. (2011). A clinical overview of centrosome amplification in human cancers. Int. J. Biol. Sci. 7, 1122-1144.
De, S., Cipriano, R., Jackson, M. W., Stark, G. R.(2009) Overexpression of kinesins mediates docetaxel resistance in breast cancer cells. Cancer Research 69(20), pp. 8035-8042
Drosopoulos K., Tang C., Chao W. C., Linardopoulos S. (2014) APC/C is an essential regulator of centrosome clustering. Nat Commun. 2014, 5: 3686 Ganem, N. J., and Compton, D. A. (2004). The Kini kinesin Kif2a is required for bipolar spindle assembly through a functional relationship with MCAK. J. Cell Biol. 166, 473-478.
Ganem, N. J., Godinho, S. A., and Pellman, D. (2009). A mechanism linking extra centrosomes to chromosomal instability. Nature 460, 278-282.
Gatlin, J. C., and Bloom, K. (2010). Microtubule motors in eukaryotic spindle assembly and maintenance. Semin. Cell Dev. Biol. 21, 248-254.
Gaulton, A., Bellis, L. J., Bento, A. P., Chambers, J., Davies, M., Hersey, A., Light, Y., McGlinchey, S., Michalovich, D., AI-Lazikani, B., and Overington, J. P. (2012). ChEMBL: a large-scale bioactivity database for drug discovery. Nucleic Acids Res. 40(Database issue), D1100-D1107.
Gordon, M. B., Howard, L., and Compton, D. A. (2001). Chromosome movement in mitosis requires microtubule anchorage at spindle poles. J. Cell Biol. 152, 425-434.
Goshima, G., N6d6lec, F., and Vale, R. D. (2005). Mechanisms for focusing mitotic spindle poles by minus end-directed motor proteins. J. Cell Biol. 171, 229-240.
Grinberg-Rashi, H., Ofek, E., Perelman, M., Skarda, J., Yaron, P., Hajduch, M., Jacob-Hirsch, J., Amariglio, N., Krupsky, M., Simansky, D. A., et al. (2009). The expression of three genes in primary non-small cell lung cancer is associated with metastatic spread to the brain. Clin. Cancer Res. 15, 1755-1761.
Hall, I. M., Noma, K., and Grewal, S. I. (2003). RNA interference machinery regulates chromosome dynamics during mitosis and meiosis in fission yeast. Proc. Natl. Acad. Sci. USA 100, 193-198.
Holdgate, G. A., and Ward, W. H. J. (2005). Measurements of binding thermodynamics in drug discovery. Drug Discov. Today 10, 1543-1550.
Huszar, D., Theoclitou, M. E., Skolnik, J., and Herbst, R. (2009). Kinesin motor proteins as targets for cancer therapy. Cancer Metastasis Rev. 28, 197-208.
Jordan, M. A., and Wilson, L. (2004). Microtubules as a target for anticancer drugs. Nat. Rev. Cancer 4, 253-265.
Klabunde, T. (2007). Chemogenomic approaches to drug discovery: similar receptors bind similar ligands. Br. J. Pharmacol. 152, 5-7.
Kleylein-Sohn, J., P6llinger, B., Ohmer, M., Hofmann, F., Nigg, E. A., Hemmings, B. A., and Wartmann, M. (2012). Acentrosomal spindle organization renders cancer cells dependent on the kinesin HSET. J. Cell Sci. 125, 5391-5402.
Kraljevic Pavelic, S., Sedic, M., Bosnjak, H., Spaventi, S., and Pavelic, K. (2011) Metastasis: new perspectives on an old problem. Mol. Cancer 10, 22.
Kramer, A., Maier, B., and Bartek, J. (2011). Centrosome clustering and chromosomal (in)stability: a matter of life and death. Mol. Oncol. 5, 324-335.
Kwon, M., Godinho, S. A., Chandhok, N. S., Ganem, N. J., Azioune, A., Thery, M., and Pellman, D. (2008). Mechanisms to suppress multipolar divisions in cancer cells with extra centrosomes. Genes Dev. 22, 2189-2203.
Li, Y., Lu, W., Chen, D., Boohaker, R. J., Zhai, L., Padmalayam, I., Wennerberg, K., Xu, B., Zhang, W. 2015) KIFC1 is a novel potential therapeutic target for breast cancer. Cancer Biology and Therapy 16(9), pp. 1316-1322
Lipinski, C. A., Lombardo, F., Dominy, B. W., and Feeney, P. J. (2001). Experimental and computational approaches to estimate solubility and permeability in drug discovery and development settings. Adv. Drug Deliv. Rev. 46, 3-26.
Luo, L., Carson, J. D., Dhanak, D., Jackson, J. R., Huang, P. S., Lee, Y., Sakowicz, R., and Copeland, R. A. (2004). Mechanism of inhibition of human KSP by monastrol: insights from kinetic analysis and the effect of ionic strength on KSP inhibition. Biochemistry 43, 15258-15266.
Mahoney, N. M., Goshima, G., Douglass, A. D., and Vale, R. D. (2006). Making microtubules and mitotic spindles in cells without functional centrosomes. Curr. Biol. 16, 564-569.
Marx, A., Hoenger, A., and Mandelkow, E. (2009). Structures of kinesin motor proteins. Cell Motil. Cytoskeleton 66, 958-966.
Mayer, T. U., Kapoor, T. M., Haggarty, S. J., King, R. W., Schreiber, S. L., and Mitchison, T. J. (1999). Small molecule inhibitor of mitotic spindle bipolarity identified in a phenotype-based screen. Science 286, 971-974.
Meunier, S., and Vernos, I. (2012). Microtubule assembly during mitosis—from distinct origins to distinct functions?J. Cell Sci. 125, 2805-2814.
Mountain, V., Simerly, C., Howard, L., Ando, A., Schatten, G., and Compton, D. A. (1999). The kinesin-related protein, HSET, opposes the activity of Eg5 and cross-links microtubules in the mammalian mitotic spindle. J. Cell Biol. 147, 351-366.
Myers and Collins, 2016: Myers S., Collins I. (2016). Recent findings and future directions for interpolar mitotic kinesin inhibitors in cancer therapy. Future Med Chem. 8: 463-89
Neres, J., Wilson, D. J., Celia, L., Beck, B. J., and Aldrich, C. C. (2008). Aryl acid adenylating enzymes involved in siderophore biosynthesis: fluorescence polarization assay, ligand specificity, and discovery of non-nucleoside inhibitors via high-throughput screening. Biochemistry 47, 11735-11749.
Niesen, F. H., Berglund, H., and Vedadi, M. (2007). The use of differential scanning fluorimetry to detect ligand interactions that promote protein stability. Nat. Protoc. 2, 2212-2221.
Nigg, E. A. (2002). Centrosome aberrations: cause or consequence of cancer progression?Nat. Rev. Cancer 2, 815-825.
Ogden, A., Rida, P. C., and Aneja, R. (2012). Let's huddle to prevent a muddle: centrosome declustering as an attractive anticancer strategy. Cell Death Differ. 19, 1255-1267.
Ogden, A., Rida, P. C., and Aneja, R. (2013). Heading off with the herd: how cancer cells might maneuver supernumerary centrosomes for directional migration. Cancer Metastasis Rev. 32, 269-287.
Rath, O., Kozielski, F. (2012) Kinesins and cancer. Nature Reviews Cancer 12(8), pp. 527-539
Pannu, V., Rida, P. C. G., Ogden, A., Turaga, R. C., Donthamsetty, S, Bowen N. J., Rudd K, Gupta M. V., Reid M. D., Cantuaria G., Walczak, C. E., Aneja, R. (2015) HSET overexpression fuels tumor progression via centrosome clustering-independent mechanisms in breast cancer patients. Oncotarget. 6: 6076-6091
Patel N. E., Weekes D., Drosopoulos K., Gazinska P., Noel E., Rashid M., Mirza H., Quist J., Braso-Maristany F., Mathew S., Ferro R., Mendes Pereira A., Prince C., Noor F., Francesch-Domenech E., Marlow R., de Rinaldis E., Grigoriadis A., Linardopoulos S., Marra P., Tutt A. N. J. (2018). Integrated genomics and functional validation identifys malignant cell specific dependencies in triple negative breast cancer. Nat. Commun. 9: 1044
Pawar, S., Donthamsetty, S., Pannu, V., Rida, P., Ogden, A., Bowen, N., Osan, R., Cantuaria, G., Aneja, R. (2014) KIFCI, a novel putative prognostic biomarker for ovarian adenocarcinomas: Delineating protein interaction networks and signaling circuitries. Journal of Ovarian Research 7(1),53
Sharp, D. J., Rogers, G. C., and Scholey, J. M. (2000). Microtubule motors in mitosis. Nature 407, 41-47.
Tillement, V., Remy, M. H., Raynaud-Messina, B., Mazzolini, L., Haren, L., and Merdes, A. (2009). Spindle assembly defects leading to the formation of a monopolar mitotic apparatus. Biol. Cell 101, 1-11.
Vichai, V., and Kirtikara, K. (2006). Sulforhodamine B colorimetric assay for cytotoxicity screening. Nat. Protoc. 1, 1112-1116.
Walczak, C. E., Verma, S., Mitchison, T. J. (1997) XCTK2: A kinesin-related protein that promotes mitotic spindle assembly in Xenopus laevis egg extracts. Journal of Cell Biology 136(4), pp. 859-870
Watts C. A., Richards F. M., Bender A., Bond P. J., Korb O., Kern O., Riddick M., Owen P., Myers R. M., Raff J., Gergely F., Jodrell D. I. (2013) Design, synthesis, and biological evaluation of an allosteric inhibitor of HSET that targets cancer cells with supernumerary centrosomes. Chemistry & Biology 20: 1399-1410
Wu J., Mikule K., Wang W., Su N., Petteruti P., Gharahdaghi F., Code E., Zhu X., Jacques K., Lai Z., Yang B., Lamb M. L. (2013) Discovery and mechanistic study of a small molecule inhibitor for motor protein KIFC1. ACS Chem Biol. 8: 2201-2208 Zhang et al., 2016: Zhang W., Zhai L., Wang Y., Boohaker R. J., Lu W., Gupta V. V., Padmalayam I., Bostwick R. J., White E. L., Ross L. J., Maddry J., Ananthan S. Discovery of a novel inhibitor of kinesin-like protein KIFC1. Biochem J. 473: 1027-1035
Yang, Z., Loncarek, J., Khodjakov, A., and Rieder, C. L. (2008). Extra centrosomes and/or chromosomes prolong mitosis in human cells. Nat. Cell Biol. 10, 748-751.
Zyss, D., and Gergely, F. (2009). Centrosome function in cancer: guilty or innocent?Trends Cell Biol. 19, 334-346.

1. Compound of the formula I, 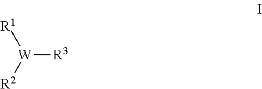
wherein
W denotes 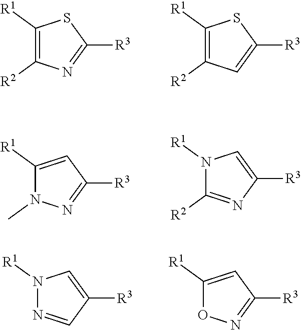
R¹ denotes Hal, CN, A, COOR⁷, CON(R⁷)₂, COA′ or Het¹,
R² denotes H, Hal, A′,
R³ denotes 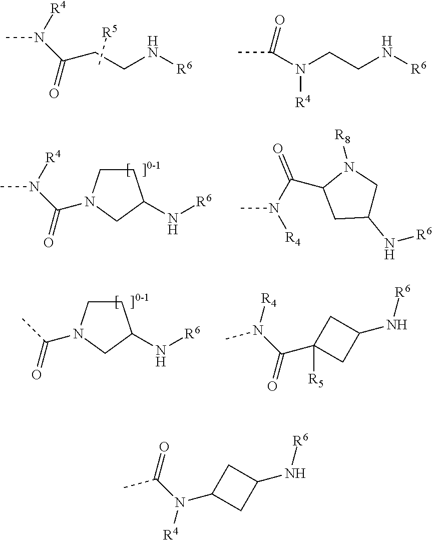
R⁴, R⁸ denote independently of one another H, unbranched or branched alkyl with 1-3 C-atoms,
R⁵ denotes H or one or more substituents selected from the group of Hal, A, OH, OCH₃, NH₂, NHCH₃, N(CH₃)₂ and CH₂CN,
R⁶ denotes benzoyl, 2-isoquinolinyl or 4-quinazolinyl, which is unsubstituted or one-, two-, or threefold substituted with Hal, A, OH,
N(R⁷)₂, COOR⁷, CN, NO₂ and/or or Het²,
A denotes unbranched or branched alkyl or cycloalkyl with 1-10 C-atoms, wherein two adjacent CH— and/or CH₂-groups may form a double bond and wherein one or two non-adjacent CH— and/or CH₂-groups may be replaced by N-, O- and/or S-atoms and wherein 1-7 H-atoms may be replaced by F or CI,
A′ denotes unbranched or branched alkyl with 1-4 C-atoms, wherein 1-7 H-atoms may be replaced by F or Cl,
Het¹ denotes oxadiazolyl, which is unsubstituted or substituted with unbranched or branched alkyl with 1-4 C-atoms,
Het² denotes oxadiazolyl, tetrazolyl, pyrazolyl or oxazolyl, which unsubstituted or substituted with unbranched or branched alkyl with 1-4 C-atoms,
R⁷ denotes H or A,
Hal denotes F, Cl, Br or I
and physiologically acceptable salts, derivatives, solvates, prodrugs and stereoisomers thereof, including mixtures thereof in all ratios.

2. Compound according to claim 1, wherein
W denotes 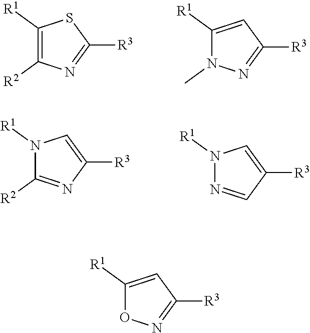
and R¹, R², R³, R⁴, R⁵, R⁶, R⁷, R⁸, A, A′, Het¹ and Het² have the meanings as above, and physiologically acceptable salts, derivatives, solvates, prodrugs and stereoisomers thereof, including mixtures thereof in all ratios.

3. Compound according to claim 1, wherein
W denotes 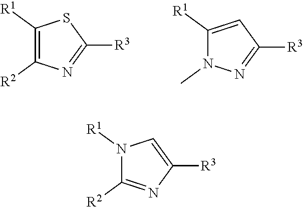
and R¹, R², R³, R⁴, R⁵, R⁶, R⁷, R⁸, A, A′, Het¹ and Het² have the meanings as above and physiologically acceptable salts, derivatives, solvates, prodrugs and stereoisomers thereof, including mixtures thereof in all ratios.

4. Compound according to claim 1, wherein
W denotes 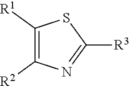
and R¹, R², R³, R⁴, R⁵, R⁶, R⁷, R⁸, A, A′, Het¹ and Het² have the meanings as above, and physiologically acceptable salts, derivatives, solvates, prodrugs and stereoisomers thereof, including mixtures thereof in all ratios.

5. Compound according to claim 1, wherein
R² denotes H or A′
and W, R¹, R³, R⁴, R⁵, R⁶, R⁷, R⁸, A, A′, Het¹ and Het² have the meanings as above and physiologically acceptable salts, derivatives, solvates, prodrugs and stereoisomers thereof, including mixtures thereof in all ratios.

6. Compound according to claim 1, wherein
R² denotes H or methyl
and W, R¹, R³, R⁴, R⁵, R⁶, R⁷, R⁸, A, A′, Het¹ and Het² have the meanings as in above, and physiologically acceptable salts, derivatives, solvates, prodrugs and stereoisomers thereof, including mixtures thereof in all ratios.

7. Compound according to claim 1, wherein
R³ denotes 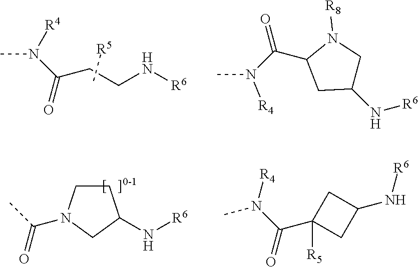
and W, R¹, R², R⁴, R⁵, R⁶, R⁷, R⁸, A, A′, Het¹ and Het² have the meanings as in above, and physiologically acceptable salts, derivatives, solvates, prodrugs and stereoisomers thereof, including mixtures thereof in all ratios.

8. Compound according to claim 1, wherein
R³ denotes 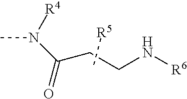
and W, R¹, R², R⁴, R⁵, R⁶, R⁷, R⁸, A, A′, Het¹ and Het² have the meanings as above and physiologically acceptable salts, derivatives, solvates, prodrugs and stereoisomers thereof, including mixtures thereof in all ratios.

9. Compound according to claim 1, wherein
R⁶ denotes benzoyl or 2-isoquinolinyl and W, R¹, R², R³, R⁴, R⁵, R⁷, R⁸, A, A′, Het¹ and Het² have the meanings as above and physiologically acceptable salts, derivatives, solvates, prodrugs and stereoisomers thereof, including mixtures thereof in all ratios.

10. Compound according to claim 1, wherein
Het² denotes oxadiazolyl or tetrazolyl and W, R¹, R², R³, R⁴, R⁵, R⁶, R⁷, R⁸, A, A′, and Het¹ have the meanings as above, and physiologically acceptable salts, derivatives, solvates, prodrugs and stereoisomers thereof, including mixtures thereof in all ratios.

11. Compound according to claim 1, wherein
Het² denotes methyl-oxadiazolyl or methyl-tetrazolyl and W, R¹, R², R³, R⁴, R⁵, R⁶, R⁷, R⁸, A, A′, and Het¹ have the meanings as above, and physiologically acceptable salts, derivatives, solvates, prodrugs and stereo-isomers thereof, including mixtures thereof in all ratios.

12. Compound selected from the group consisting of:

| | |
|---|---|
| 1 | Ethyl-4-methyl-2-(3-(3- |
| | methylbenzamido)propanamido)thiazole-5- |
| | carboxylate |
| 2 | Ethyl-4-methyl-2-(3-(3-(pyrrolidin-1- |
| | yl)benzamido)propanamido)thiazole-5-carboxylate |
| 3 | Ethyl-2-(3-(3-(1H-pyrazol-1-yl)benzamido)propanamido)-4- |
| | methylthiazole-5-carboxylate |
| 4 | Ethyl-4-methyl-2-(3-(3-(5-methyl-1,2,4-oxadiazol-3- |
| | yl)benzamido)propanamido)thiazole-5-carboxylate |
| 5 | Ethyl-4-methyl-2-(3-(5,6,7,8-tetrahydronaphthalene-2- |
| | carboxamido)propanamido)thiazole-5-carboxylate |
| 6 | Ethyl-4-methyl-2-(3-(3-(oxazol-5- |
| | yl)benzamido)propanamido)thiazole-5-carboxylate |
| 7 | Ethyl-4-methyl-2-(3-(3- |
| | morpholinobenzamido)propanamido)thiazole- |
| | 5-carboxylate |
| 8 | Ethyl-(S)-2-(2-hydroxy-3-(3- |
| | methylbenzamido)propanamido)-4- |
| | methylthiazole-5-carboxylate |
| 9 | Ethyl-2-[3-[(3- |
| | methoxycarbonylbenzoyl)amino]propanoylamino]-4- |
| | methyl-thiazole-5-carboxylate |
| 10 | Ethyl-2-[3-[(3-fluoro-5-methoxycarbonyl-benzoyl)amino]- |
| | propanoylamino]-4-methyl-thiazole-5-carboxylate |
| 11 | Ethyl-2-[3-[(2-fluoro-3-methoxycarbonyl-benzoyl)amino]- |
| | propanoylamino]-4-methyl-thiazole-5-carboxylate |
| 12 | Ethyl-2-[3-[(2-chloro-5-methoxycarbonyl-benzoyl)amino]- |
| | propanoylamino]-4-methyl-thiazole-5-carboxylate |
| 13 | Ethyl-2-[3-[(3-chloro-5-methoxycarbonyl-benzoyl)amino]- |
| | propanoylamino]-4-methyl-thiazole-5-carboxylate |
| 14 | Ethyl-2-[3-[(3-methoxycarbonyl-4-methyl-benzoyl)amino]- |
| | propanoylamino]-4-methyl-thiazole-5-carboxylate |
| 15 | Ethyl-2-[3-[(3-methoxycarbonyl-5-methyl-benzoyl)amino]- |
| | propanoylamino]-4-methyl-thiazole-5-carboxylate |
| 16 | Ethyl-2-[3-[(3-methoxy-5-methoxycarbonyl-benzoyl)amino]- |
| | propanoylamino]-4-methyl-thiazole-5-carboxylate |
| 17 | Ethyl-2-[3-[(3-cyano-5-methoxycarbonyl-benzoyl)amino]- |
| | propanoylamino]-4-methyl-thiazole-5-carboxylate |
| 18 | Ethyl-2-[3-[(3-bromo-5-methoxycarbonyl-benzoyl)amino]- |
| | propanoylamino]-4-methyl-thiazole-5-carboxylate |
| 19 | Ethyl-2-[3-[(3-ethyl-5-methoxycarbonyl-benzoyl)amino]- |
| | propanoylamino]-4-methyl-thiazole-5-carboxylate |
| 20 | Ethyl-2-[3-[(3-hydroxy-5-methoxycarbonyl-benzoyl)amino]- |
| | propanoylamino]-4-methyl-thiazole-5-carboxylate |
| 21 | Ethyl-4-methyl-2-[3-[[3-(5-methyl-1,2,4-oxadiazol-3- |
| | yl)benzoyl]-amino]propylamino]thiazole-5-carboxylate |
| 22 | Ethyl-2-[3-[[3-methoxycarbonyl-5-[(E)- |
| | styryl]benzoyl]amino]propanoyl-amino]-4-methyl-thiazole-5- |
| | carboxylate |
| 23 | Ethyl-2-(3-(3-(1,2,4-oxadiazol-3- |
| | yl)benzamido)propanamido)-4- |
| | methylthiazole-5-carboxylate |
| 24 | Ethyl-2-(3-(3-chloro-5-(5-methyl- |
| | 1,2,4-oxadiazol-3-yl)benzamido)- |
| | propanamido)-4-methylthiazole-5-carboxylate |
| 25 | Propyl-2-(3-(3-chloro-5-(5-methyl- |
| | 1,2,4-oxadiazol-3-yl)benzamido)- |
| | propanamido)-4-methylthiazole-5-carboxylate |
| 26 | Propyl-4-methyl-2-(3-(3-methyl- |
| | 5-(5-methyl-1,2,4-oxadiazol-3- |
| | yl)benzamido)propanamido)thiazole-5-carboxylate |
| 27 | Propyl-1-methyl-3-(3-(3-methyl- |
| | 5-(5-methyl-1,2,4-oxadiazol-3- |
| | yl)benzamido)propanamido)-1H-pyrazole-5-carboxylate |
| 28 | Propyl-2-(3-(3-cyano-5- |
| | (trifluoromethyl)benzamido)propanamido)-4- |
| | methylthiazole-5-carboxylate |
| 29 | Propyl-2-(3-(3-(tert-butyl)-5-(methoxycarbonyl)benzamido)- |
| | propanamido)-4-methylthiazole-5-carboxylate |
| 30 | Propyl-2-(3-(3-cyano-5- |
| | (methoxycarbonyl)benzamido)propanamido)- |
| | 4-methylthiazole-5-carboxylate |
| 31 | N-[3-[(5-tert-butyl-4-methyl- |
| | thiazol-2-yl)amino]-3-oxo-propyl]-3- |
| | chloro-5-(5-methyl-1,2,4-oxadiazol-3-yl)benzamide |
| 32 | Propyl-4-methyl-2-(3-(3-(5-methyl-1,2,4-oxadiazol-3-yl)-5- |
| | (trifluoromethyl)benzamido)propanamido)thiazole- |
| | 5-carboxylate |
| 33 | Propyl-2-[3-[(3-methoxycarbonyl-5-nitro-benzoyl)amino]- |
| | propanoylamino]-4-methyl-thiazole-5-carboxylate |
| 34 | Ethyl-4-methyl-2-(3-(3-(3-methyl-1,2,4-oxadiazol-5- |
| | yl)benzamido)propanamido)thiazole-5-carboxylate |
| 35 | Cyclopropyl-4-methyl-2-(3-(3-(5-methyl-1,2,4-oxadiazol-3- |
| | yl)benzamido)propanamido)thiazole-5-carboxylate |
| 36 | Ethyl-2-(3-(3-(5-ethyl-1,2,4-oxadiazol- |
| | 3-yl)benzamido)propanamido)- |
| | 4-methylthiazole-5-carboxylate |
| 37 | Ethyl-4-methyl-2-(3-(3-(5-methyl- |
| | 1,3,4-oxadiazol-2-yl)benzamido)- |
| | propanamido)thiazole-5-carboxylate |
| 38 | Ethyl-4-methyl-2-(3-(3-(2-methyl- |
| | 2H-tetrazol-5-yl)benzamido)- |
| | propanamido)thiazole-5-carboxylate |
| 39 | Tert-butyl-4-methyl-2-[3-[[3-(2- |
| | methyltetrazol-5-yl)benzoyl]amino]- |
| | propanoylamino]thiazole-5-carboxylate |
| 40 | Isopropyl-4-methyl-2-[3-[[3-(2- |
| | methyltetrazol-5-yl)benzoyl]amino]- |
| | propanoylamino]thiazole-5-carboxylate |
| 41 | N-[3-[(5-tert-butyl-4-methyl-thiazol- |
| | 2-yl)amino]-3-oxo-propyl]-3-(2- |
| | methyltetrazol-5-yl)benzamide |
| 42 | Ethyl-2-(3-(3- |
| | (ethoxycarbonyl)benzamido)propanamido)-4- |
| | methylthiazole-5-carboxylate |
| 43 | Ethyl4-methyl-2-(3-(3-methyl- |
| | 5-(5-methyl-1,2,4-oxadiazol-3- |
| | yl)benzamido)propanamido)thiazole-5-carboxylate |
| 44 | Propyl-2-(3-(3-(methoxycarbonyl)- |
| | 5-(5-methyl-1,2,4-oxadiazol-3- |
| | yl)benzamido)propanamido)-4-methylthiazole-5-carboxylate |
| 45 | Ethyl-4-methyl-2-[3-[[7-(5-methyl-1,2,4-oxadiazol-3-yl)-1- |
| | isoquinolyl]amino]propanoylamino]thiazole-5-carboxylate |
| 46 | Methyl-4-methyl-2-[3-[[7-(5-methyl-1,2,4-oxadiazol-3-yl)-1- |
| | isoquinolyl]amino]propanoylamino]thiazole-5-carboxylate |
| 47 | N-(5-tert-butyl-4-methyl-thiazol-2-yl)-3-[[6-(5-methyl-1,2,4- |
| | oxadiazol-3-yl)quinazolin-4-yl]amino]propanamide |
| 48 | Tert-butyl-4-methyl-2-((1s,3s)-3- |
| | ((6-(5-methyl-1,2,4-oxadiazol-3- |
| | yl)quinazolin-4-yl)amino)cyclobutane-1- |
| | carboxamido)thiazole-5-carboxylate |
| 49 | Tert-butyl-4-methyl-2-((1s,3s)- |
| | N-methyl-3-((6-(5-methyl-1,2,4- |
| | oxadiazol-3-yl)quinazolin-4-yl)amino)cyclobutane-1- |
| | carboxamido)thiazole-5-carboxylate |
| 50 | N-(5-tert-butyl-4-methyl-thiazol-2-yl)-3-[(7-cyano-1- |
| | isoquinolyl)amino]-propanamide |
| 51 | 3-[(7-cyano-1-isoquinolyl)amino]- |
| | N-[4-methyl-5-[1-(trifluoromethyl)- |
| | cyclopropyl]thiazol-2-yl]propanamide |
| 52 | 3-[[7-(5-methyl-1,2,4-oxadiazol- |
| | 3-yl)-1-isoquinolyl]amino]-N-[4- |
| | methyl-5-[1-(trifluoromethyl)cyclopropyl]thiazol- |
| | 2-yl]propanamide |
| 53 | N-(5-tert-butyl-4-methyl-thiazol- |
| | 2-yl)-3-[(7-cyano-5-fluoro-1- |
| | isoquinolyl)amino]propanamide |
| 54 | Propyl-4-methyl-2-(3-((7-(5-methyl- |
| | 1,2,4-oxadiazol-3-yl)isoquinolin- |
| | 1-yl)amino)propanamido)thiazole-5-carboxylate |
| 55 | N-(5-cyanothiazol-2-yl)-3-((7-(5-methyl-1,2,4-oxadiazol-3- |
| | yl)isoquinolin-1-yl)amino)propanamide |
| 56 | 3-((7-(5-methyl-1,2,4-oxadiazol- |
| | 3-yl)isoquinolin-1-yl)amino)-N-(1- |
| | methyl-5-(trifluoromethyl)-1H-pyrazol-3-yl)propanamide |
| 57 | 3-((7-(5-methyl-1,2,4-oxadiazol- |
| | 3-yl)isoquinolin-1-yl)amino)-N-(1- |
| | methyl-5-pentyl-1H-pyrazol-3-yl)propanamide |
| 58 | N-(5-(tert-butyl)-4-methylthiazol-2- |
| | yl)-3-((7-(5-methyl-2H-tetrazol-2- |
| | yl)isoquinolin-1-yl)amino)propanamide |
| 59 | N-(3-((5-(tert-butyl)-4-methylthiazol- |
| | 2-yl)amino)-3-oxopropyl)-3-(5- |
| | methyl-2H-tetrazol-2-yl)benzamide |
| 60 | (S)-N-(1-((5-(tert-butyl)-4-methylthiazol-2-yl)amino)-6- |
| | (methylamino)-1-oxohexan-3-yl)-3-(5-methyl-2H-tetrazol-2- |
| | yl)benzamide |
| 61 | Ethyl-2-(3-(3-(5-methyl-1,2,4-oxadiazol-3-yl)benzamido)- |
| | propanamido)thiazole-5-carboxylate |
| 62 | Methyl-4-methyl-2-(3-(3-(5-methyl- |
| | 1,2,4-oxadiazol-3-yl)benzamido)- |
| | propanamido)thiazole-5-carboxylate |
| 63 | Propyl-4-methyl-2-(3-(3-(5-methyl-1,2,4-oxadiazol-3- |
| | yl)benzamido)propanamido)thiazole-5-carboxylate |
| 64 | N-(3-((5-butyl-4-methylthiazol-2- |
| | yl)amino)-3-oxopropyl)-3-(5-methyl- |
| | 1,2,4-oxadiazol-3-yl)benzamide |
| 65 | Ethyl-4-methyl-2-(3-methyl-3-(3-(5-methyl-1,2,4-oxadiazol- |
| | 3-yl)benzamido)butanamido)thiazole-5-carboxylate |
| 66 | Isopropyl-4-methyl-2-(3-(3-(5-methyl-1,2,4-oxadiazol-3- |
| | yl)benzamido)-propanamido)thiazole-5-carboxylate |
| 67 | 2-methoxyethyl-4-methyl-2-(3-(3-(5-methyl-1,2,4-oxadiazol- |
| | 3-yl)benzamido)propanamido)thiazole-5-carboxylate |
| 68 | Isobutyl-4-methyl-2-(3-(3-(5-methyl- |
| | 1,2,4-oxadiazol-3-yl)benzamido)- |
| | propanamido)thiazole-5-carboxylate |
| 69 | 4-methoxybutyl-4-methyl-2-(3-(3-(5-methyl-1,2,4-oxadiazol- |
| | 3-yl)benzamido)propanamido)thiazole-5-carboxylate |
| 70 | Propyl-4-methyl-2-((2-((7-(5-methyl- |
| | 1,2,4-oxadiazol-3-yl)isoquinolin- |
| | 1-yl)amino)ethyl)carbamoyl)thiazole-5-carboxylate |
| 71 | Ethyl-1-methyl-3-((2-((7-(5-methyl- |
| | 1,2,4-oxadiazol-3-yl)isoquinolin- |
| | 1-yl)amino)ethyl)carbamoyl)-1H-pyrazole-5-carboxylate |
| 72 | Propyl-1-methyl-3-((2-((7-(5-methyl- |
| | 1,2,4-oxadiazol-3-yl)isoquinolin- |
| | 1-yl)amino)ethyl)carbamoyl)-1H-pyrazole-5-carboxylate |
| 73 | 1-methyl-3-(3-((7-(5-methyl-1,2,4-oxadiazol- |
| | 3-yl)isoquinolin-1-yl)amino)propanamido)- |
| | N-propyl-1H-pyrazole-5-carboxamide |
| 74 | Propyl-4-methyl-2-(methyl(2-((7-(5-methyl-1,2,4-oxadiazol- |
| | 3-yl)isoquinolin-1-yl)amino)ethyl)carbamoyl)thiazole- |
| | 5-carboxylate |
| 75 | N-(5-(tert-butyl)-4-methylthiazol-2-yl)-3-((7-(5-methyl-1,2,4- |
| | oxadiazol-3-yl)isoquinolin-1-yl)amino)propanamide |
| 76 | N-(5-ethyl-1-methyl-1H-pyrazol-3-yl)-3-((7-(5-methyl-1,2,4- |
| | oxadiazol-3-yl)isoquinolin-1-yl)amino)propanamide |
| 77 | N-(5-(tert-butyl)isoxazol-3-yl)-3- |
| | ((7-(5-methyl-1,2,4-oxadiazol-3- |
| | yl)isoquinolin-1-yl)amino)propanamide |
| 78 | N-(5-cyclopropylisoxazol-3-yl)- |
| | 3-((7-(5-methyl-1,2,4-oxadiazol-3- |
| | yl)isoquinolin-1-yl)amino)propanamide |
| 79 | N-(5-ethylisoxazol-3-yl)-3-((7-(5-methyl-1,2,4-oxadiazol-3- |
| | yl)isoquinolin-1-yl)amino)propanamide |
| 80 | N-(5-(tert-butyl)thiazol-2-yl)-3-((7-(5-methyl-1,2,4- |
| | oxadiazol-3-yl)isoquinolin-1-yl)amino)propanamide |
| 81 | N-(5-(tert-butyl)-4-ethylthiazol-2-yl)- |
| | 3-((7-(5-methyl-1,2,4-oxadiazol- |
| | 3-yl)isoquinolin-1-yl)amino)propanamide |
| 82 | N-(5-(tert-butyl)-1-methyl-1H- |
| | pyrazol-3-yl)-3-((7-(5-methyl-1,2,4- |
| | oxadiazol-3-yl)isoquinolin-1-yl)amino)propanamide |
| 83 | N-[5-(3-methoxyoxetan-3-yl)-4- |
| | methyl-thiazol-2-yl]-3-[[7-(5-methyl- |
| | 1,2,4-oxadiazol-3-yl)-1-isoquinolyl]amino]propanamide |
| 84 | 3-[[7-(5-methyl-1,2,4-oxadiazol- |
| | 3-yl)-1-isoquinolyl]amino]-N-[4- |
| | methyl-5-(3-propoxyoxetan-3-yl)thiazol-2-yl]propanamide |
| 85 | N,4-dimethyl-2-[3-[[7-(5-methyl-1,2,4-oxadiazol-3-yl)-1- |
| | isoquinolyl]amino]propanoylamino]- |
| | N-propyl-thiazole-5-carboxamide |
| 86 | N-[5-(cyclohexen-1-yl)thiazol-2-yl]- |
| | 3-[[7-(5-methyl-1,2,4-oxadiazol- |
| | 3-yl)-1-isoquinolyl]amino]propanamide |
| 87 | N-[5-(1-methoxycyclohexyl)thiazol- |
| | 2-yl]-3-[[7-(5-methyl-1,2,4- |
| | oxadiazol-3-yl)-1-isoquinolyl]amino]propanamide |
| 88 | Ethyl-4-isopropyl-2-[3-[[3-(5-methyl-1,2,4-oxadiazol-3- |
| | yl)benzoyl]amino]propanoylamino]thiazole-5-carboxylate |
| 89 | Tertbutyl-4-methyl-2-(methyl(2- |
| | ((7-(5-methyl-1,2,4-oxadiazol-3- |
| | yl)isoquinolin-1-yl)amino)ethyl)carbamoyl)thiazole- |
| | 5-carboxylate |
| 90 | Methyl-4-ethyl-2-[3-[[3-(5-methyl-1,2,4-oxadiazol-3- |
| | yl)benzoyl]amino]propanoylamino]thiazole-5-carboxylate |
| 91 | Ethyl-2-[3-[[3-(5-methyl-1,2,4-oxadiazol-3-yl)benzoyl]amino]- |
| | propanoylamino]-4-(trifluoromethyl)thiazole-5-carboxylate |
| 92 | Ethyl-4-ethyl-2-[3-[[3-(5-methyl-1,2,4-oxadiazol-3- |
| | yl)benzoyl]amino]propanoylamino]thiazole-5-carboxylate |
| 93 | N-[3-[[5-[(E)-2-cyclopentylvinyl]- |
| | 4-methyl-thiazol-2-yl]amino]-3- |
| | oxo-propyl]-3-(5-methyl-1,2,4-oxadiazol-3-yl)benzamide |
| 94 | N-[3-[5-[(E)-3-methoxyprop-1-enyl]- |
| | 4-methyl-thiazol-2-yl]amino]-3- |
| | oxo-propyl]-3-(5-methyl-1,2,4-oxadiazol-3-yl)benzamide |
| 95 | 3-(5-methyl-1,2,4-oxadiazol-3-yl)-N-[3-[4-methyl-5-(1- |
| | phenylvinyl)thiazol-2-yl]amino]-3-oxo-propyl]benzamide |
| 96 | 3-[[7-(5-methyl-1,2,4-oxadiazol-3- |
| | yl)-1-isoquinolyl]amino]-N-[5- |
| | (1,1,2,2,2-pentafluoroethyl)thiazol- |
| | 2-yl]cyclobutanecarboxamide |
| 97 | N-(5-bromo-4-methyl-thiazol-2-yl)-3- |
| | [[7-(5-methyl-1,2,4-oxadiazol-3- |
| | yl)-1-isoquinolyl]amino]cyclobutanecarboxamide |
| 98 | (1s,3s)-N-(5-(tert-butyl)-4-methylthiazol- |
| | 2-yl)-3-((7-(5-methyl-1,2,4- |
| | oxadiazol-3-yl)isoquinolin-1-yl)amino)cyclobutane- |
| | 1-carboxamide |
| 99 | Propyl-4-methyl-2-((1s,3s)-3-((7-(5-methyl-1,2,4- |
| | oxadiazol-3-yl)isoquinolin-1-yl)amino)cyclobutane- |
| | 1-carboxamido)thiazole-5-carboxylate |
| 100 | (1s,3s)-N-(5-(tert-butyl)isoxazol-3-yl)-3-((7-(5-methyl-1,2,4- |
| | oxadiazol-3-yl)isoquinolin-1-yl)amino)cyclobutane- |
| | 1-carboxamide |
| 101 | (1s,3s)-3-((7-(5-methyl-1,2,4-oxadiazol- |
| | 3-yl)isoquinolin-1-yl)amino)- |
| | N-(1-methyl-5-pentyl-1H-pyrazol-3- |
| | yl)cyclobutane-1-carboxamide |
| 102 | (1s,3s)-N-(5-isobutyl-4-methylthiophen- |
| | 2-yl)-3-((7-(5-methyl-1,2,4- |
| | oxadiazol-3-yl)isoquinolin-1-yl)amino)cyclobutane- |
| | 1-carboxamide |
| 103 | (1s,3s)-3-((7-(5-methyl-1,2,4-oxadiazol- |
| | 3-yl)isoquinolin-1-yl)amino)- |
| | N-(4-methyl-5-(trifluoromethyl)thiazol-2-yl)cyclobutane-1- |
| | carboxamide |
| 104 | Tert-butyl-4-methyl-2-((1s,3s)-3- |
| | (3-(5-methyl-1,2,4-oxadiazol-3- |
| | yl)benzamido)cyclobutane-1-carboxamido)thiazole- |
| | 5-carboxylate |
| 105 | Tert-butyl-2-((1s,3s)-3-(3-(1,2,4-oxadiazol-3-yl)benzamido)- |
| | cyclobutane-1-carboxamido)-4-methylthiazole-5-carboxylate |
| 106 | Tert-butyl-4-methyl-2-((1s,3s)-3- |
| | (3-(5-methyl-1,3,4-oxadiazol-2- |
| | yl)benzamido)cyclobutane-1-carboxamido)thiazole- |
| | 5-carboxylate |
| 107 | Tert-butyl-4-methyl-2-((1s,3s)-3-(3-(2-methyl-2H-tetrazol-5- |
| | yl)benzamido)cyclobutane-1-carboxamido)thiazole- |
| | 5-carboxylate |
| 108 | Tert-butyl-2-((1s,3s)-3-((7- |
| | chloroisoquinolin-1-yl)amino)cyclobutane- |
| | 1-carboxamido)-4-methylthiazole-5-carboxylate |
| 109 | Tert-butyl-4-methyl-2-((1s,3s)-3-((7- |
| | (trifluoromethyl)isoquinolin-1- |
| | yl)amino)cyclobutane-1-carboxamido)thiazole-5-carboxylate |
| 110 | Tert-butyl-4-methyl-2-[[3-[[7-(5- |
| | methyl-1,2,4-oxadiazol-3-yl)-1- |
| | isoquinolyl]amino]cyclobutanecarbonyl]amino]thiazole- |
| | 5-carboxylate |
| 111 | Tert-butyl-4-methyl-2-[methyl-[3-[[7- |
| | (5-methyl-1,2,4-oxadiazol-3-yl)- |
| | 1-isoquinolyl]amino]cyclobutanecarbonyl]amino]thiazole-5- |
| | carboxylate |
| 112 | Tert-butyl-4-chloro-2-[[3-[[7- |
| | (5-methyl-1,2,4-oxadiazol-3-yl)-1- |
| | isoquinolyl]amino]cyclobutanecarbonyl]amino]thiazole- |
| | 5-carboxylate |
| 113 | N-tert-butyl-4-chloro-2-[[3-[[7- |
| | (5-methyl-1,2,4-oxadiazol-3-yl)-1- |
| | isoquinolyl]amino]cyclobutanecarbonyl]amino]thiazole-5- |
| | carboxamide |
| 114 | [(1R)-2,2,2-trifluoro-1-methyl-ethyl]- |
| | 4-methyl-2-[[3-[[7-(5-methyl- |
| | 1,2,4-oxadiazol-3-yl)-1- |
| | isoquinolyl]amino]cyclobutanecarbonyl]amino]- |
| | thiazole-5-carboxylate |
| 115 | [(1S)-2,2,2-trifluoro-1-methyl-ethyl]-4-methyl-2- |
| | [[3-[7-(5-methyl-1,2,4-oxadiazol-3-yl)-1- |
| | isoquinolyl]amino]cyclobutanecarbonyl]amino]- |
| | thiazole-5-carboxylate |
| 116 | Ethyl-4-(difluoromethyl)-2-[[3-[7- |
| | (5-methyl-1,2,4-oxadiazol-3-yl)-1- |
| | isoquinolyl]amino]cyclobutanecarbonyl]amino]thiazole- |
| | 5-carboxylate |
| 117 | N-(5-tert-butyl-4-methyl-thiazol-2-yl)-3-[(7-methyl-1- |
| | isoquinolyl)amino]-cyclobutanecarboxamide |
| 118 | 3-(5-methyl-1,2,4-oxadiazol-3-yl)-N- |
| | (3-((4-methyl-5-(3-methyl-1,2,4- |
| | oxadiazol-5-yl)thiazol-2-yl)amino)-3-oxopropyl)benzamide |
| 119 | 3-((7-(5-methyl-1,2,4-oxadiazol-3- |
| | yl)isoquinolin-1-yl)amino)-N-(5-(5- |
| | propyl-1,2,4-oxadiazol-3-yl)thiazol-2-yl)propanamide |
| 120 | Proyl-1-methyl-3-(3-(3-(5-methyl-1,2,4-oxadiazol-3- |
| | yl)benzamido)propanamido)-1H-pyrazole-5-carboxylate |
| 121 | Propyl-4-methyl-2-(N-methyl-3- |
| | (3-(5-methyl-1,2,4-oxadiazol-3- |
| | yl)benzamido)propanamido)thiazole-5-carboxylate |
| 122 | Cyclopentyl-4-methyl-2-(3-(3-(5-methyl-1,2,4-oxadiazol-3- |
| | yl)benzamido)propanamido)thiazole-5-carboxylate |
| 123 | Hexyl-4-methyl-2-(3-(3-(5-methyl-1,2,4-oxadiazol-3- |
| | yl)benzamido)propanamido)thiazole-5-carboxylate |
| 124 | Cyclohexyl-4-methyl-2-(3-(3-(5-methyl-1,2,4-oxadiazol-3- |
| | yl)benzamido)propanamido)thiazole-5-carboxylate |
| 125 | 4-aminobutyl-4-methyl-2-(3-(3-(5-methyl-1,2,4-oxadiazol-3- |
| | yl)benzamido)propanamido)thiazole-5-carboxylate |
| 126 | 3-(4-methoxyphenyl)propyl-4-methyl-2-(3-(3-(5-methyl- |
| | 1,2,4-oxadiazol-3-yl)benzamido)propanamido)thiazole- |
| | 5-carboxylate |
| 127 | 4-(2-methoxyethoxy)butyl-4-methyl-2-(3-(3-(5-methyl- |
| | 1,2,4-oxadiazol-3-yl)benzamido)propanamido)thiazole- |
| | 5-carboxylate |
| 128 | Methyl-2-methyl-5-[3-[[3-(5-methyl-1,2,4-oxadiazol-3- |
| | yl)benzoyl]amino]propanoylamino]pyrazole-3-carboxylate |
| 129 | 3-(5-methyl-1,2,4-oxadiazol-3-yl)-N-(3-((4-methyl-5- |
| | (pentan-2-yl)thiazol-2-yl)amino)-3-oxopropyl)benzamide |
| 130 | 4-acetamidobutyl-4-methyl-2-(3-(3-(5-methyl-1,2,4- |
| | oxadiazol-3-yl)benzamido)propanamido)thiazole- |
| | 5-carboxylate |
| 131 | Propyl-1-methyl-3-(3-((7-(5-methyl- |
| | 1,2,4-oxadiazol-3-yl)isoquinolin- |
| | 1-yl)amino)propanamido)-1H-pyrazole-5-carboxylate |
| 132 | Tert-butyl-4-methyl-2-(3-(3-(5-methyl-1,2,4-oxadiazol-3- |
| | yl)benzamido)propanamido)thiazole-5-carboxylate |
| 133 | Ethyl-3-methyl-5-[3-[[3-(5-methyl-1,2,4-oxadiazol-3- |
| | yl)benzoyl]amino]propanoylamino]thiophene-2-carboxylate |
| 134 | Ethyl-4-methyl-2-(3-(3-(5-methyl-1,2,4- |
| | oxadiazol-3-yl)benzamido)-N- |
| | propylpropanamido)thiazole-5-carboxylate |
| 135 | Ethyl-3-(3-((7-(5-methyl-1,2,4-oxadiazol-3-yl)isoquinolin-1- |
| | yl)amino)propanamido)-1H-pyrazole-5-carboxylate. |
| 136 | 6-hydroxyhexyl-4-methyl-2-(3-(3- |
| | (5-methyl-1,2,4-oxadiazol-3-yl)-5- |
| | (trifluoromethyl)benzamido)propanamido)thiazole- |
| | 5-carboxylate |
| 137 | 5-butyl-4-methyl-N-(2-((7-(5-methyl- |
| | 1,2,4-oxadiazol-3-yl)isoquinolin- |
| | 1-yl)amino)ethyl)thiazole-2-carboxamide |
| 138 | Cyclopentyl-1-methyl-3-(3-((7-(5-methyl-1,2,4-oxadiazol-3- |
| | yl)isoquinolin-1-yl)amino)propanamido)- |
| | 1H-pyrazole-5-carboxylate |
| 139 | 3-{[3-(5-methyl-1,2,4-oxadiazol-3-yl)phenyl]formamido}-N- |
| | [4-methyl-5-(trifluoromethyl)-1,3-thiazol-2-yl]propenamide |
| 140 | Ethyl-2-((2-(3-(5-ethyl-1,2,4-oxadiazol-3-yl)benzamido)ethyl)- |
| | carbamoyl)-4-methylthiazole-5-carboxylate |
| 141 | Ethyl-4-methyl-2-((2-(3-(5-methyl-1,2,4-oxadiazol-3- |
| | yl)benzamido)ethyl)carbamoyl)thiazole-5-carboxylate |
| 142 | Ethyl-(R)-4-methyl-2-(3-(3-(5-methyl-1,2,4-oxadiazol-3- |
| | yl)benzamido)butanamido)thiazole-5-carboxylate |
| 143 | Ethyl-(S)-4-methyl-2-(3-(3-(5-methyl-1,2,4-oxadiazol-3- |
| | yl)benzamido)butanamido)thiazole-5-carboxylate |
| 144 | Propyl-2-(2-(4-(3-chloro-5-(5-methyl-1,2,4-oxadiazol-3- |
| | yl)benzamido)-1-methylpiperidin-4- |
| | yl)acetamido)-4-methylthiazole-5- |
| | carboxylate |
| 145 | Ethyl-4-methyl-2-(2-methyl-3-(3-(5-methyl-1,2,4-oxadiazol-3- |
| | yl)benzamido)propanamido)thiazole-5-carboxylate |
| 146 | 4-(3-((5-(ethoxycarbonyl)-4-methylthiazol-2-yl)amino)-1-(3-(5- |
| | methyl-1,2,4-oxadiazol-3-yl)benzamido)- |
| | 3-oxopropyl)piperidin-1-ium |
| 147 | 4-(3-((5-(ethoxycarbonyl)-4-methylthiazol-2-yl)amino)-1-(3-(5- |
| | methyl-1,2,4-oxadiazol-3-yl)benzamido)-3-oxopropyl)-1- |
| | methylpiperidin-1-ium |
| 148 | Ethyl-2-[[(3R)-4-amino-3-[[3-(5-methyl-1,2,4-oxadiazol-3- |
| | yl)benzoyl]amino]butanoyl]amino]-4- |
| | methyl-thiazole-5-carboxylate |
| 149 | Ethyl-4-methyl-2-[[(3S,4R)-4-[[3-(5-methyl-1,2,4-oxadiazol-3- |
| | yl)benzoyl]amino]pyrrolidine-3-carbonyl]amino]thiazole-5- |
| | carboxylate; |
| 150 | Ethyl-(S)-2-(6-amino-3-(3-(5-methyl-1,2,4-oxadiazol-3- |
| | yl)benzamido)hexanamido)-4-methylthiazole-5-carboxylate |
| 151 | Propyl-(S)-2-(6-amino-3-(3-(5-methyl-1,2,4-oxadiazol-3- |
| | yl)benzamido)hexanamido)-4-methylthiazole-5-carboxylate |
| 152 | Propyl-4-methyl-2-[[(3S)-6- |
| | (methylamino)-3-[[3-(5-methyl-1,2,4- |
| | oxadiazol-3-yl)benzoyl]amino]hexanoyl]amino]thiazole- |
| | 5-carboxylate |
| 153 | Propyl-(R)-4-methyl-2-(3-(3-(5-methyl-1,2,4-oxadiazol-3- |
| | yl)benzamido)-6-(methylamino)hexanamido)thiazole- |
| | 5-carboxylate |
| 154 | Tertbutyl-4-methyl-2-[[(3S)-6- |
| | (methylamino)-3-[[3-(5-methyl-1,2,4- |
| | oxadiazol-3-yl)benzoyl]amino]hexanoyl]amino]thiazole- |
| | 5-carboxylate |
| 155 | Cyclopentyl-S)-4-methyl-2-(3-(3-(5-methyl-1,2,4-oxadiazol-3- |
| | yl)benzamido)-6-(methylamino)hexanamido)thiazole- |
| | 5-carboxylate |
| 156 | Isopropyl-(S)-4-methyl-2-(3-(3-(5-methyl-1,2,4-oxadiazol-3- |
| | yl)benzamido)-6-(methylamino)hexanamido)thiazole- |
| | 5-carboxylate |
| 157 | (S)-N-(1-((5-(tert-butyl)-4-methylthiazol-2-yl)amino)-6- |
| | (methylamino)-1-oxohexan-3-yl)-3-chloro-5- |
| | (5-methyl-1,2,4-oxadiazol-3-yl)benzamide |
| 158 | Propyl-(S)-2-(6-(dimethylamino)-3-(3- |
| | (5-methyl-1,2,4-oxadiazol-3- |
| | yl)benzamido)hexanamido)-4-methylthiazole-5-carboxylate |
| 159 | Propyl-(S)-4-methyl-2-(3-(3-(5-methyl-1,2,4-oxadiazol-3- |
| | yl)benzamido)-6-morpholinohexanamido)thiazole- |
| | 5-carboxylate |
| 160 | Propyl-(S)-2-(2-amino-3-(3-(5-methyl-1,2,4-oxadiazol-3- |
| | yl)benzamido)propanamido)-4-methylthiazole-5-carboxylate |
| 161 | (S)-N-(2-amino-3-((5-(tert-butyl)-4-ethylthiazol-2-yl)amino)-3- |
| | oxopropyl)-3-(5-methyl-1,2,4-oxadiazol-3-yl)benzamide |
| 162 | Propyl-(S)-2-(5-(dimethylamino)-3- |
| | (3-(5-methyl-1,2,4-oxadiazol-3- |
| | yl)benzamido)pentanamido)-4-methylthiazole-5-carboxylate |
| 163 | Propyl-(S)-2-(6-amino-3-((7-(5-methyl-1,2,4-oxadiazol-3- |
| | yl)isoquinolin-1-yl)amino)hexanamido)-4-methylthiazole-5- |
| | carboxylate |
| 164 | (S)-6-amino-N-(5-(tert-butyl)-4- |
| | methylthiazol-2-yl)-3-((7-(5-methyl- |
| | 1,2,4-oxadiazol-3-yl)isoquinolin-1-yl)amino)hexanamide |
| 165 | Cyclopentyl-(S)-4-methyl-2-(3-((7-(5-methyl-1,2,4- |
| | oxadiazol-3-yl)isoquinolin-1-yl)amino)-6- |
| | (methylamino)hexanamido)thiazole-5- |
| | carboxylate |
| 166 | 1,3-difluoropropan-2-yl (S)-4-methyl-2-(3-((7-(5-methyl-1,2,4- |
| | oxadiazol-3-yl)isoquinolin-1-yl)amino)-6-(methylamino)- |
| | hexanamido)thiazole-5-carboxylate |
| 167 | Isopropyl-(S)-4-methyl-2-(3-((7-(5-methyl-1,2,4-oxadiazol-3- |
| | yl)isoquinolin-1-yl)amino)-6- |
| | (methylamino)hexanamido)thiazole-5- |
| | carboxylate |
| 168 | Tertbutyl-(S)-4-methyl-2-(3-((7-(5-methyl-1,2,4-oxadiazol-3- |
| | yl)isoquinolin-1-yl)amino)-6- |
| | (methylamino)hexanamido)thiazole-5- |
| | carboxylate |
| 169 | (S)-4-methyl-2-(3-((7-(5-methyl-1,2,4- |
| | oxadiazol-3-yl)isoquinolin-1- |
| | yl)amino)-6-(methylamino)hexanamido)thiazole-5-carboxylic |
| 170 | 3,3-difluorocyclopentyl-4-methyl- |
| | 2-((S)-3-((7-(5-methyl-1,2,4- |
| | oxadiazol-3-yl)isoquinolin-1-yl)amino)- |
| | 6-(methylamino)hexanamido)- |
| | thiazole-5-carboxylate |
| 171 | (S)-N-(5-(tert-butyl)-4-methylthiazol- |
| | 2-yl)-3-((7-(5-methyl-1,2,4- |
| | oxadiazol-3-yl)isoquinolin-1-yl)amino)- |
| | 6-(methylamino)hexanamide |
| 172 | Propyl-(S)-4-methyl-2-(3-((7-(5-methyl-1,2,4-oxadiazol-3- |
| | yl)isoquinolin-1-yl)amino)-6- |
| | (methylamino)hexanamido)thiazole-5- |
| | carboxylate |
| 173 | Propyl-(R)-2-(2-amino-3-(3-(5-methyl-1,2,4-oxadiazol-3- |
| | yl)benzamido)propanamido)-4-methylthiazole-5-carboxylate |
| 174 | Propyl-(S)-2-(2-amino-3-((7-(5-methyl-1,2,4-oxadiazol-3- |
| | yl)isoquinolin-1-yl)amino)propanamido)-4-methylthiazole-5- |
| | carboxylate |
| 175 | Propyl-(S)-2-(2-(dimethylamino)-3- |
| | (3-(5-methyl-1,2,4-oxadiazol-3- |
| | yl)benzamido)propanamido)-4-methylthiazole-5-carboxylate |
| 176 | Propyl-(R)-2-(2-hydroxy-3-((7-(5-methyl-1,2,4-oxadiazol-3- |
| | yl)isoquinolin-1-yl)amino)propanamido)-4-methylthiazole-5- |
| | carboxylate |
| 177 | Tertbutyl-(R)-2-(2-hydroxy-3-((7-(5-methyl-1,2,4-oxadiazol-3- |
| | yl)isoquinolin-1-yl)amino)propanamido)-4-methylthiazole-5- |
| | carboxylate |
| 178 | Propyl-(S)-2-(2-hydroxy-3-((7-(5-methyl-1,2,4-oxadiazol-3- |
| | yl)isoquinolin-1-yl)amino)propanamido)-4-methylthiazole-5- |
| | carboxylate |
| 179 | (S)-2-hydroxy-N-(5-isobutyl-4- |
| | methylthiazol-2-yl)-3-((7-(5-methyl- |
| | 1,2,4-oxadiazol-3-yl)isoquinolin-1-yl)amino)propanamide |
| 180 | Tertbutyl-(S)-2-(2-hydroxy-3-((7-(5-methyl-1,2,4-oxadiazol-3- |
| | yl)isoquinolin-1-yl)amino)propanamido)-4-methylthiazole-5- |
| | carboxylate |
| 181 | Tertbutyl-(S)-2-(2-hydroxy-3-((7-(1-methyl-1H-pyrazol-4- |
| | yl)isoquinolin-1-yl)amino)propanamido)-4-methylthiazole-5- |
| | carboxylate |
| 182 | (S)-2-hydroxy-3-((7-(5-methyl-1,2,4- |
| | oxadiazol-3-yl)isoquinolin-1- |
| | yl)amino)-N-(1-methyl-5-(5-propyl- |
| | 1,2,4-oxadiazol-3-yl)-1H-pyrazol- |
| | 3-yl)propanamide |
| 183 | (S)-2-hydroxy-N-(1-methyl-5-(5- |
| | propyl-1,2,4-oxadiazol-3-yl)-1H- |
| | pyrazol-3-yl)-3-((7-methylisoquinolin- |
| | 1-yl)amino)propanamide |
| 184 | Tertbutyl-(R)-2-(2-hydroxy-3-((7-methylisoquinolin-1- |
| | yl)amino)propanamido)-4-methylthiazole-5-carboxylate |
| 185 | Tertbutyl-(S)-2-(2-hydroxy-3-((7-methylisoquinolin-1- |
| | yl)amino)propanamido)-4-methylthiazole-5-carboxylate |
| 186 | Propyl(S)-2-(2-methoxy-3-((7-(5-methyl-1,2,4-oxadiazol-3- |
| | yl)isoquinolin-1-yl)amino)propanamido)-4-methylthiazole-5- |
| | carboxylate |
| 187 | (S)-N-(5-(tert-butyl)-4-methylthiazol- |
| | 2-yl)-2-hydroxy-3-((7-(5-methyl- |
| | 1,2,4-oxadiazol-3-yl)isoquinolin-1-yl)amino)propanamide |
| 188 | (S)-2-hydroxy-3-((7-(5-methyl- |
| | 1,2,4-oxadiazol-3-yl)isoquinolin-1- |
| | yl)amino)-N-(1-methyl-5-pentyl-1H-pyrazol- |
| | 3-yl)propanamide |
| 189 | (R)-2-hydroxy-3-((7-(5-methyl- |
| | 1,2,4-oxadiazol-3-yl)isoquinolin-1- |
| | yl)amino)-N-(1-methyl-5-pentyl-1H-pyrazol- |
| | 3-yl)propanamide |
| 190 | (R)-N-(5-(tert-butyl)-4-methylthiazol-2-yl)- |
| | 2-hydroxy-3-((7-(5-methyl-1,2,4-oxadiazol-3- |
| | yl)isoquinolin-1-yl)amino)propanamide |
| 191 | (R)-2-hydroxy-N-(5-isobutyl-4- |
| | methylthiazol-2-yl)-3-((7-(5-methyl- |
| | 1,2,4-oxadiazol-3-yl)isoquinolin-1-yl)amino)propanamide |
| 192 | (R)-2-hydroxy-3-((7-(5-methyl-1,2,4- |
| | oxadiazol-3-yl)isoquinolin-1- |
| | yl)amino)-N-(4-methyl-5-(1- |
| | (trifluoromethyl)cyclopropyl)thiazol-2- |
| | yl)propanamide |
| 193 | Tertbutyl-2-[(3S)-4-cyano-3-{[3-(5-methyl-1,2,4-oxadiazol-3- |
| | yl)phenyl]formamido}butanamido]-4-methyl-1,3-thiazole-5- |
| | carboxylate |
| 194 | Tertbutyl-2-[(3R)-4-cyano-3-{[3-(5-methyl-1,2,4-oxadiazol-3- |
| | yl)phenyl]formamido}butanamido]-4-methyl-1,3-thiazole-5- |
| | carboxylate |
| 195 | Propan-2-yl-2-[(3S)-4-cyano-3-{[3-(5-methyl-1,2,4- |
| | oxadiazol-3-yl)phenyl]formamido}butanamido]-4-methyl- |
| | 1,3-thiazole-5-carboxylate |
| 196 | Propan-2-yl-2-[(3R)-4-cyano-3-{[3-(5-methyl-1,2,4- |
| | oxadiazol-3-yl)phenyl]formamido}butanamido]-4-methyl- |
| | 1,3-thiazole-5-carboxylate |
| 197 | Methyl-2-[(3S)-4-cyano-3-{[3-(5-methyl-1,2,4-oxadiazol-3- |
| | yl)phenyl]formamido}butanamido]-4-methyl-1,3-thiazole-5- |
| | carboxylate |
| 198 | Methyl-2-[(3R)-4-cyano-3-{[3-(5-methyl-1,2,4-oxadiazol-3- |
| | yl)phenyl]formamido}butanamido]-4-methyl-1,3-thiazole-5- |
| | carboxylate |
| 199 | Propan-2-yl-2-[(3S)-4-cyano-N-methyl-3-{[3-(5-methyl-1,2,4- |
| | oxadiazol-3-yl)phenyl]formamido}butanamido]- |
| | 4-methyl-1,3-thiazole-5-carboxylate |
| 200 | Propan-2-yl-2-[(3R)-4-cyano-N-methyl-3-{[3-(5-methyl- |
| | 1,2,4-oxadiazol-3-yl)phenyl]formamido}butanamido]- |
| | 4-methyl-1,3-thiazole-5-carboxylate |
| 201 | Tertbutyl-2-[(3S)-4-cyano-3-{[3-(1- |
| | methyl-1H-pyrazol-4-yl)phenyl]- |
| | formamido}butanamido]-4-methyl- |
| | 1,3-thiazole-5-carboxylate |
| 202 | Tertbutyl-2-[(3R)-4-cyano-3-{[3-(1-methyl-1H- |
| | pyrazol-4-yl)phenyl]-formamido}butanamido]-4- |
| | methyl-1,3-thiazole-5-carboxylate |
| 203 | Propan-2-yl-2-[(3S)-4-cyano-3-{[3-(1-methyl-1H-pyrazol-4- |
| | yl)phenyl]formamido}butanamido]-4-methyl-1,3-thiazole-5- |
| | carboxylate |
| 204 | Propan-2-yl-2-[(3R)-4-cyano-3-{[3-(1-methyl-1H-pyrazol-4- |
| | yl)phenyl]formamido}butanamido]-4-methyl-1,3-thiazole-5- |
| | carboxylate |
| 205 | Propan-2-yl-2-[(3S)-4-cyano-N-methyl- |
| | 3-{[3-(1-methyl-1H-pyrazol-4- |
| | yl)phenyl]formamido}butanamido]-4-methyl-1,3-thiazole-5- |
| | carboxylate |
| 206 | Propan-2-yl-2-[(3R)-4-cyano-N-methyl- |
| | 3-{[3-(1-methyl-1H-pyrazol-4- |
| | yl)phenyl]formamido}butanamido]-4-methyl-1,3-thiazole-5- |
| | carboxylate |
| 207 | Propan-2-yl-2-[(3S)-4-cyano-3-[(3-cyanophenyl)formamido]- |
| | butanamido]-4-methyl-1,3-thiazole-5-carboxylate |
| 208 | Propan-2-yl-2-[(3S)-4-cyano-3-[(7-cyanoisoquinolin-1- |
| | yl)amino]butanamido]-4-methyl-1,3-thiazole-5-carboxylate |
| 209 | Propan-2-yl-2-[(3R)-4-cyano-3-[(7-cyanoisoquinolin-1- |
| | yl)amino]butanamido]-4-methyl-1,3-thiazole-5-carboxylate |
| 210 | Propan-2-yl-2-[(3S)-4-cyano-3-{[7-(1-methyl-1H-pyrazol-4- |
| | yl)isoquinolin-1-yl]amino}butanamido]- |
| | 4-methyl-1,3-thiazole-5-carboxylate |
| 211 | Tertbutyl-2-[(3S)-4-cyano-3-{[6-(1- |
| | methyl-1H-pyrazol-4-yl)pyridin-2- |
| | yl]formamido}butanamido]-4-methyl- |
| | 1,3-thiazole-5-carboxylate |
| 212 | Tertbutyl-2-[(3R)-4-cyano-3-{[6-(1- |
| | methyl-1H-pyrazol-4-yl)pyridin-2- |
| | yl]formamido}butanamido]-4-methyl- |
| | 1,3-thiazole-5-carboxylate |
| 213 | Propan-2-yl-2-[(3S)-4-cyano-3-{[6-(1-methyl-1H-pyrazol-4- |
| | yl)pyridin-2-yl]formamido}butanamido]-4- |
| | methyl-1,3-thiazole-5-carboxylate |
| 214 | Propan-2-yl-2-[(3R)-4-cyano-3-{[6-(1-methyl-1H-pyrazol-4- |
| | yl)pyridin-2-yl]formamido}butanamido]-4-methyl- |
| | 1,3-thiazole-5-carboxylate |
| 215 | Ethyl-4-methyl-2-(3-{[3-(5-methyl-1,2,4-oxadiazol-3- |
| | yl)phenyl]formamido}pentanamido)- |
| | 1,3-thiazole-5-carboxylate |
| 216 | (3R)-4-{[5-(methoxycarbonyl)-4- |
| | methyl-1,3-thiazol-2-yl]carbamoyl}- |
| | 3-{[3-(5-methyl-1,2,4-oxadiazol-3- |
| | yl)phenyl]formamido}butanoic |
| 217 | (3S)-4-{[5-(methoxycarbonyl)-4-methyl-1,3-thiazol-2- |
| | yl](methyl)carbamoyl}-3-{[3-(5-methyl-1,2,4-oxadiazol-3- |
| | yl)phenyl]formamido}butanoic |
| 218 | Tertbutyl-2-((1s,3s)-N-ethyl-3- |
| | ((7-(5-methyl-1,2,4-oxadiazol-3- |
| | yl)isoquinolin-1-yl)amino)cyclobutanecarboxamido)- |
| | 4-methylthiazole-5-carboxylate |
| 219 | Ethyl-4-methyl-2-(N-methyl-3-(3-(5-methyl-1,2,4-oxadiazol-3- |
| | yl)benzamido)propanamido)thiazole-5-carboxylate |
| 220 | Propyl-4-methyl-2-(((1s,3s)-3-((7-(5-methyl-1,2,4-oxadiazol-3- |
| | yl)isoquinolin-1-yl)amino)cyclobutyl)carbamoyl)thiazole-5- |
| | carboxylate |
| 221 | (1r,3r)-N-(5-(tert-butyl)-4-methylthiazol-2-yl)- |
| | 1-hydroxy-3-((7-(5-methyl-1,2,4-oxadiazol-3- |
| | yl)isoquinolin-1-yl)amino)cyclobutane-1- |
| | carboxamide |
| 222 | Tertbutyl-2-((1s,3s)-1-fluoro-3-((7-(5-methyl-1,2,4- |
| | oxadiazol-3-yl)isoquinolin-1-yl)amino)cyclobutane- |
| | 1-carboxamido)-4-methylthiazole-5-carboxylate |
| 223 | Tertbutyl-2-((1r,3r)-1-fluoro-3-((7-(5-methyl-1,2,4- |
| | oxadiazol-3-yl)isoquinolin-1-yl)amino)cyclobutane- |
| | 1-carboxamido)-4-methylthiazole-5-carboxylate |
| 224 | Tertbutyl-4-methyl-2-((1s,3s)-1-methyl-3-((7-(5-methyl-1,2,4- |
| | oxadiazol-3-yl)isoquinolin-1-yl)amino)cyclobutane-1- |
| | carboxamido)thiazole-5-carboxylate |
| 225 | Tertbutyl-4-methyl-2-[[(3R)-3-[[7-(5- |
| | methyl-1,2,4-oxadiazol-3-yl)-1- |
| | isoquinolyl]amino]pyrrolidine-1-carbonyl]amino]thiazole-5- |
| | carboxylate |
| 226 | Tertbutyl-4-methyl-2-[[(3S)-3-[[7- |
| | (5-methyl-1,2,4-oxadiazol-3-yl)-1- |
| | isoquinolyl]amino]pyrrolidine-1-carbonyl]amino]thiazole-5- |
| | carboxylate |
| 227 | Tertbutyl-4-methyl-2-[methyl-[(3S)- |
| | 3-[[7-(5-methyl-1,2,4-oxadiazol- |
| | 3-yl)-1-isoquinolyl]amino]pyrrolidine- |
| | 1-carbonyl]amino]thiazole-5- |
| | carboxylate |
| 228 | Tertbutyl-4-methyl-2-[[3-[[3-(5-methyl-1,2,4-oxadiazol-3- |
| | yl)benzoyl]amino]azetidine-1-carbonyl]amino]thiazole- |
| | 5-carboxylate |
| 229 | Tertbutyl-4-methyl-2-[methyl-[3-[[3- |
| | (5-methyl-1,2,4-oxadiazol-3- |
| | yl)benzoyl]amino]azetidine-1-carbonyl]amino]thiazole- |
| | 5-carboxylate |
| 230 | Tertbutyl-4-methyl-2-[[3-[[7-(5-methyl- |
| | 1,2,4-oxadiazol-3-yl)-1-isoquinolyl]amino]azetidine- |
| | 1-carbonyl]amino]thiazole-5-carboxylate |
| 231 | Tertbutyl-4-methyl-2-[methyl-[3-[[7- |
| | (5-methyl-1,2,4-oxadiazol-3-yl)- |
| | 1-isoquinolyl]aminoJazetidine-1-carbonyl]amino]thiazole-5- |
| | carboxylate |
| 232 | N-(5-(tert-butyl)-4-methylthiazol-2-yl)-3-((7-(5-methyl-1,2,4- |
| | oxadiazol-3-yl)isoquinolin-1-yl)amino)azetidine- |
| | 1-carboxamide |
| 233 | Propyl-2-(3-(3-chloro-5-(5-methyl-1,2,4- |
| | oxadiazol-3-yl)benzamido)- |
| | N-methylpropanamido)-4-methylthiazole-5-carboxylate |
| 234 | Propyl-4-methyl-2-((2S,4S)-1-methyl- |
| | 4-(3-(5-methyl-1,2,4-oxadiazol- |
| | 3-yl)benzamido)pyrrolidine-2-carboxamido)thiazole- |
| | 5-carboxylate. |
| 235 | Propyl-4-methyl-2-((2R,4R)-1-methyl- |
| | 4-(3-(5-methyl-1,2,4-oxadiazol- |
| | 3-yl)benzamido)pyrrolidine-2-carboxamido)thiazole- |
| | 5-carboxylate |
| 236 | (2S,4S)-N-(5-(tert-butyl)-4-methylthiazol-2-yl)-1-methyl- |
| | 4-((7-(5-methyl-1,2,4-oxadiazol-3-yl)isoquinolin- |
| | 1-yl)amino)pyrrolidine-2-carboxamide |
| 237 | Propyl-4-methyl-2-((2S,4S)-1-methyl-4-((7-(5-methyl- |
| | 1,2,4-oxadiazol-3-yl)isoquinolin-1-yl)amino)pyrrolidine- |
| | 2-carboxamido)thiazole-5-carboxylate |
| 238 | (2S,4S)-1-methyl-4-((7-(5-methyl-1,2,4-oxadiazol-3- |
| | yl)isoquinolin-1-yl)amino)-N-(1-methyl-5-pentyl-1H- |
| | pyrazol-3-yl)pyrrolidine-2-carboxamide |
| 239 | Propyl-4-methyl-2-((2S,4S)-4-((7-(5-methyl-1,2,4- |
| | oxadiazol-3-yl)isoquinolin-1-yl)amino)pyrrolidine- |
| | 2-carboxamido)thiazole-5-carboxylate |
| 240 | Propyl-(S)-4-methyl-2-(3-(3-(5-methyl-1,2,4-oxadiazol-3- |
| | yl)benzamido)pyrrolidine-1-carbonyl)thiazole-5-carboxylate |
| 241 | Propyl-(R)-4-methyl-2-(3-(3-(5-methyl-1,2,4-oxadiazol-3- |
| | yl)benzamido)pyrrolidine-1-carbonyl)thiazole-5-carboxylate |
| 242 | Propyl-4-methyl-2-[(3S)-3-[[3-(2-methyltetrazol-5- |
| | yl)benzoyl]amino]pyrrolidine-1-carbonyl]thiazole- |
| | 5-carboxylate |
| 243 | Propyl-(S)-4-methyl-2-(3-((7-(5-methyl-1,2,4-oxadiazol-3- |
| | yl)isoquinolin-1-yl)amino)pyrrolidine-1-carbonyl)thiazole-5- |
| | carboxylate |
| 244 | Propyl-(R)-4-methyl-2-(3-((7-(5-methyl-1,2,4-oxadiazol-3- |
| | yl)isoquinolin-1-yl)amino)pyrrolidine-1-carbonyl)thiazole-5- |
| | carboxylate |
| 245 | Propyl-(S)-1-methyl-3-(3-((7-(5-methyl-1,2,4-oxadiazol-3- |
| | yl)isoquinolin-1-yl)amino)pyrrolidine-1-carbonyl)- |
| | 1H-pyrazole-5-carboxylate |
| 246 | (S)-(5-(tert-butyl)-4-methylthiazol-2-yl)(3- |
| | ((7-(5-methyl-1,2,4-oxadiazol-3-yl)isoquinolin- |
| | 1-yl)amino)pyrrolidin-1-yl)methanone |
| 247 | (S)-(5-cyclopropyl-1-methyl-1H-pyrazol- |
| | 3-yl)(3-((7-(5-methyl-1,2,4-oxadiazol-3- |
| | yl)isoquinolin-1-yl)amino)pyrrolidin-1-yl)methanone |
| 248 | (S)-(3-((7-(5-methyl-1,2,4-oxadiazol-3-yl)isoquinolin-1- |
| | yl)amino)pyrrolidin-1-yl)(1-methyl-5-phenyl-1H-pyrazol-3- |
| | yl)methanone |
| 249 | (S)-(5-isopropyl-1-methyl-1H-pyrazol-3-yl)(3- |
| | ((7-(5-methyl-1,2,4-oxadiazol-3-yl)isoquinolin- |
| | 1-yl)amino)pyrrolidin-1-yl)methanone |
| 250 | S)-(1H-imidazol-5-yl)(3-((7-(5-methyl-1,2,4-oxadiazol-3- |
| | yl)isoquinolin-1-yl)amino)pyrrolidin-1-yl)methanone |
| 251 | (5-methylisoxazol-3-yl)-[(3S)-3-[[7- |
| | (5-methyl-1,2,4-oxadiazol-3-yl)-1- |
| | isoquinolyl]amino]pyrrolidin-1-yl]methanone |
| 252 | (1-Tert-butylpyrazol-4-yl)-[(3S)-3- |
| | [[7-(5-methyl-1,2,4-oxadiazol-3- |
| | yl)-1-isoquinolyl]amino]pyrrolidin-1-yl]methanone |
| 253 | (S)-(5-isopropyl-1H-pyrazol-3-yl)(3- |
| | ((7-(5-methyl-1,2,4-oxadiazol-3- |
| | yl)isoquinolin-1-yl)amino)pyrrolidin-1-yl)methanone |
| 254 | (S)-(5-(tert-butyl)-1H-pyrazol-3-yl)(3- |
| | ((7-(5-methyl-1,2,4-oxadiazol-3- |
| | yl)isoquinolin-1-yl)amino)pyrrolidin-1-yl)methanone |
| 255 | Propyl-4-methyl-2-((2S,3R)-2-methyl-3-((7-(5-methyl-1,2,4- |
| | oxadiazol-3-yl)isoquinolin-1-yl)amino)pyrrolidine-1- |
| | carbonyl)thiazole-5-carboxylate |
| 256 | Tertbutyl-2-((3R,4R)-3-fluoro-4-((7-(5-methyl-1,2,4- |
| | oxadiazol-3-yl)isoquinolin-1-yl)amino)pyrrolidine-1- |
| | carbonyl)-4-methylthiazole-5-carboxylate |
| 257 | Propyl-2-methyl-4-(3-{[7-(5-methyl- |
| | 1,2,4-oxadiazol-3-yl)isoquinolin- |
| | 1-yl]amino}propanamido)-1H-imidazole-1-carboxylate |
| 258 | Propan-2-yl-2-methyl-4-(3-{[7-(5-methyl- |
| | 1,2,4-oxadiazol-3-yl)isoquinolin-1- |
| | yl]amino}propanamido)-1H-imidazole-1-carboxylate |
| 259 | Ethyl-2-methyl-4-(3-{[7-(5-methyl- |
| | 1,2,4-oxadiazol-3-yl)isoquinolin-1- |
| | yl]amino}propanamido)-1H-imidazole-1-carboxylate |
| 260 | Propyl-4-(3-{[7-(5-methyl-1,2,4-oxadiazol-3-yl)isoquinolin- |
| | 1-yl]amino}propanamido)-1H-imidazole-1-carboxylate |
| 261 | Ethyl-2-methyl-4-(3-{[7-(1-methyl- |
| | 1H-pyrazol-4-yl)isoquinolin-1- |
| | yl]amino}propanamido)-1H-imidazole-1-carboxylate |
| 262 | Propyl-2-methyl-4-(3-{[7-(1-methyl- |
| | 1H-pyrazol-4-yl)isoquinolin-1- |
| | yl]amino}propanamido)-1H-imidazole-1-carboxylate |
| 263 | Propyl-2-methyl-4-(3-{[3-(5-methyl-1,2,4-oxadiazol-3- |
| | yl)phenyl]formamido}propanamido)- |
| | 1H-imidazole-1-carboxylate |
| 264 | Propan-2-yl-2-methyl-4-(3-{[3-(5-methyl-1,2,4- |
| | oxadiazol-3-yl)phenyl]formamido}propanamido)- |
| | 1H-imidazole-1-carboxylate |
| 265 | Ethyl-2-methyl-4-(3-{[3-(5-methyl-1,2,4-oxadiazol-3- |
| | yl)phenyl]formamido}propanamido)- |
| | 1H-imidazole-1-carboxylate |
| 266 | Propyl-4-(3-{[3-(5-methyl-1,2,4-oxadiazol-3- |
| | yl)phenyl]formamido}propanamido)- |
| | 1H-imidazole-1-carboxylate |
| 267 | Propyl-4-[(2S)-2-hydroxy-3-{[3-(1-methyl-1H-pyrazol-4- |
| | yl)phenyl]formamido}propanamido]-2-methyl- |
| | 1H-imidazole-1-carboxylate |
| 268 | Propyl-4-[(2R)-2-hydroxy-3-{[3-(1-methyl-1H-pyrazol-4- |
| | yl)phenyl]formamido}propanamido]-2-methyl- |
| | 1H-imidazole-1-carboxylate |
| 269 | Propan-2-yl-4-[(2S)-2-hydroxy-3-{[3-(1-methyl-1H- |
| | pyrazol-4-yl)phenyl]formamido}propanamido]-2- |
| | methyl-1H-imidazole-1-carboxylate |
| 270 | Propan-2-yl4-[(2R)-2-hydroxy-3-{[3-(1-methyl-1H- |
| | pyrazol-4-yl)phenyl]formamido}propanamido]-2- |
| | methyl-1H-imidazole-1-carboxylate |
| 271 | Ethyl-4-[(2S)-2-hydroxy-3-{[3-(1-methyl-1H-pyrazol-4- |
| | yl)phenyl]formamido}propanamido]-2-methyl- |
| | 1H-imidazole-1-carboxylate |
| 272 | Tertbutyl-4-(3-{[7-(5-methyl-1,2,4- |
| | oxadiazol-3-yl)isoquinolin-1- |
| | yl]amino}propanamido)-1H-pyrazole-1-carboxylate |
| 273 | Propyl-4-(3-{[7-(5-methyl-1,2,4-oxadiazol-3-yl)isoquinolin- |
| | 1-yl]amino}propanamido)-1H-pyrazole-1-carboxylate |
| 274 | Tertbutyl-4-(3-{[3-(5-methyl-1,2,4-oxadiazol-3- |
| | yl)phenyl]formamido}propanamido)- |
| | 1H-pyrazole-1-carboxylate |
| 275 | Propyl-4-(3-{[3-(5-methyl-1,2,4-oxadiazol-3- |
| | yl)phenyl]formamido}propanamido)-1H-pyrazole- |
| | 1-carboxylate |
| 276 | Propyl-4-methyl-2-(3-{[6-(5-methyl- |
| | 1,2,4-oxadiazol-3-yl)pyridin-2- |
| | yl]formamido}propanamido)-1,3-thiazole-5-carboxylate |
| 277 | Tertbuyl-4-methyl-2-(3-{[6-(5-methyl- |
| | 1,2,4-oxadiazol-3-yl)pyridin-2- |
| | yl]formamido}propanamido)-1,3-thiazole-5-carboxylate |
| 278 | Propan-2-yl-4-methyl-2-(3-{[6-(5-methyl-1,2,4-oxadiazol-3- |
| | yl)pyridin-2-yl]formamido}propanamido)- |
| | 1,3-thiazole-5-carboxylate |
| 279 | Tertbutyl-2-(3-{[2-fluoro-3-(5-methyl-1,2,4-oxadiazol-3- |
| | yl)phenyl]formamido}propanamido)-4-methyl-1,3-thiazole-5- |
| | carboxylate |
| 280 | Propyl-4-methyl-2-[3-[(7-methyl-1- |
| | isoquinolyl)amino]propanoyl- |
| | amino]thiazole-5-carboxylate |

and physiologically acceptable salts, derivatives, solvates, prodrugs and stereoisomers thereof, including mixtures thereof in all ratios.

13. Pharmaceutical preparation comprising at least one compound according to claim 1 and/or physiologically acceptable salts, derivatives, solvates, prodrugs and stereoisomers thereof, including mixtures thereof in all ratios.

14. Pharmaceutical preparation according to claim 13 comprising further excipients and/or adjuvants.

15. Pharmaceutical preparation comprising at least one compound according to claim 1 and/or physiologically acceptable salts, derivatives, solvates, prodrugs and stereoisomers thereof, including mixtures thereof in all ratios, and at least one further medicament active compound.

16. Process for the preparation of a pharmaceutical preparation, characterised in that a compound according to claim 1 and/or one of its physiologically acceptable salts, derivatives, solvates, prodrugs and stereoisomers, including mixtures thereof in all ratios, is brought into a suitable dosage form together with a solid, liquid or semi-liquid excipient or adjuvant.

17. A method for the treatment and/or prophylaxis of physiological and/or pathophysiological states, comprising administering to a subject in need thereof an effective amount of at least one compound according to claim 1 and/or one of its physiologically acceptable salts, derivatives, solvates, prodrugs and stereoisomers, including mixtures thereof in all ratios.

18. The method according to claim 17, wherein the physiological and/or pathophysiological states is selected from the group consisting of hyperproliferative diseases and disorders and cancer.

19. (canceled)

20. The method according to claim 18, wherein the cancer is selected from the group consisting of acute and chronic lymphocytic leukemia, acute granulocytic leukemia, adrenal cortex cancer, bladder cancer, brain cancer, breast cancer, cervical hyperplasia, cervical cancer, chorio cancer, chronic granulocytic leukemia, colon cancer, endometrial cancer, esophageal cancer, essential thrombocytosis, genitourinary carcinoma, glioma, glioblastoma, hairy cell leukemia, head and neck carcinoma, Hodgkin's disease, Kaposi's sarcoma, lung carcinoma, lymphoma, malignant carcinoid carcinoma, malignant hypercalcemia, malignant melanoma, malignant pancreatic insulinoma, medullary thyroid carcinoma, melanoma, multiple myeloma, mycosis fungoides, myeloid and lymphocytic leukemia, neuroblastoma, non-Hodgkin's lymphoma, non-small cell lung cancer, osteogenic sarcoma, ovarian carcinoma, pancreatic carcinoma, polycythemia vera, primary brain carcinoma, primary macroglobulinemia, prostatic cancer, renal cell cancer, rhabdomyosarcoma, skin cancer, small-cell lung cancer, soft-tissue sarcoma, squamous cell cancer, stomach cancer, testicular cancer, thyroid cancer and Wilms' tumor.

21. Set (kit) consisting of separate packs of
a) an effective amount of a compound according to claim 1 and/or physiologically acceptable salts, derivatives, solvates, prodrugs and stereoisomers thereof, including mixtures thereof in all ratios, and
b) an effective amount of a further medicament active compound.
